(19) United States
(12) Patent Application Publication
(10) Pub. No.: US 2024/0366612 A1
(43) Pub. Date: Nov. 7, 2024

(54) COMPOUNDS AND METHODS FOR MODULATING NUCLEIC ACID SPLICING

(71) Applicant: REMIX THERAPEUTICS INC., Watertown, MA (US)

(72) Inventors: Dominic Reynolds, Stoneham, MA (US); Michael W. Seiler, Belmont, MA (US); Anant A. Agrawal, Waltham, MA (US); Frederic Vaillancourt, Newton, MA (US); Peter Smith, Arlington, MA (US); Sudeep Prajapati, Somerville, MA (US); Allen T. Hopper, Lexington, MA (US); Stepan Vyskocil, Benesov (CZ); Benoît Moreau, Newton, MA (US)

(21) Appl. No.: 18/700,515

(22) PCT Filed: Oct. 13, 2022
(86) PCT No.: PCT/US2022/078079

§ 371 Date: Apr. 11, 2024

Related U.S. Application Data
(60) Provisional application No. 63/255,078, filed on Oct. 13, 2021.
(60) Provisional application No. 63/255,076, filed on Oct. 13, 2021.

(51) Int. Cl. A61K 31/519; A61K 31/437; A61K 31/5025; A61P 35/00; C07D 495/04; C07D 513/04; C07D 519/00

(52) U.S. Cl. CPC A61K 31/519; A61K 31/437; A61K 31/5025; A61P 35/00; C07D 495/04; C07D 513/04; C07D 519/00

(57) ABSTRACT

The present disclosure features compounds and related compositions that, inter alia, modulate nucleic acid splicing, e.g., splicing of a pre-mRNA, as well as methods of use thereof.

### CLAIM OF PRIORITY

This application claims priority to U.S. Application No. 63/255,078, filed on Oct. 13, 2021, and U.S. Application No. 63/255,076, filed on Oct. 13, 2021. The disclosure of each of the foregoing applications is incorporated herein by reference in its entirety.

### BACKGROUND

Alternative splicing is a major source of protein diversity in higher eukaryotes and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17(4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof. In an embodiment, the compounds described herein are compounds of Formulas (I), (II) or (III) and pharmaceutically acceptable salts, solvates, hydrates, tautomers, or stereoisomers thereof.

The present disclosure additionally provides methods of using the compounds of the disclosure (e.g., compounds of Formulas (I), (II), or (III) and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formulas (I), (II) or (III), and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formulas (I), (II) or (III), and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formulas (I), (II) or (III), and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In one aspect, the present disclosure provides compounds of Formula (I):

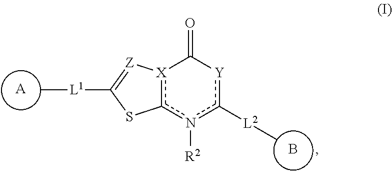

or a pharmaceutically acceptable salt, solvate, hydrate, tautomer, or stereoisomer thereof, wherein each of A, B, L¹, L², X, Y, Z, R², and subvariables thereof are defined as described herein.

In another aspect, the present disclosure provides compounds of Formula (II):

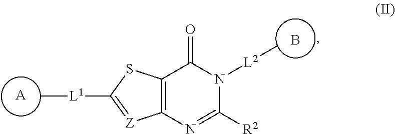

or a pharmaceutically acceptable salt, solvate, hydrate, tautomer, or stereoisomer thereof, wherein each of A, B, L¹, L², Z, R², and subvariables thereof are defined as described herein.

In another aspect, the present disclosure provides compounds of Formula (III):

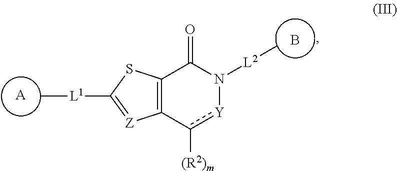

or a pharmaceutically acceptable salt, solvate, hydrate, tautomer, or stereoisomer thereof, wherein each of A, B, L¹, L², Y, Z, R², m, and subvariables thereof are defined as described herein.

In another aspect, the present invention provides pharmaceutical compositions comprising a compound of Formulas (I), (II) or (III), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formulas (I), (II) or (III), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formulas (I), (II) or (III), or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formulas (I), (II) or (III), or a pharmaceutically acceptable salt, solvate, hydrate, tautomer, or stereoisomer thereof. Modulation of splicing may comprise impacting any step involved in splicing and may include an event upstream or downstream of a splicing event.

For example, in some embodiments, the compound of Formulas (I), (II) or (III) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formulas (I), (II) or (III)alters a target nucleic acid (e.g., DNA or RNA, e.g., a precursor RNA, e.g., a pre-mRNA), target protein, or combination thereof. In some embodiments, the compound of Formulas (I), (II) or (III) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formulas (I), (II) or (III), e.g., in a healthy or diseased cell or tissue). In some embodiments, the presence of a compound of Formulas (I), (II) or (III) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formulas (I), (II) or (III), e.g., in a healthy or diseased cell or tissue).

In another aspect, the present disclosure provides methods for preventing and/or treating a disease, disorder, or condition in a subject by administering a compound of Formulas (I), (II) or (III), or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formulas (I), (II) or (III), or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formulas (I), (II) or (III) or a pharmaceutically acceptable salt, solvate, hydrate, tautomer, or stereoisomer thereof in a biological sample or subject. In another aspect, the present disclosure provides methods of altering the isoform of a target protein with a compound of Formulas (I), (II) or (III), or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formulas (I), (II) or (III) to a biological sample, a cell, or a subject comprises inhibition of cell growth or induction of cell death.

In another aspect, the present disclosure provides compositions for use in preventing and/or treating a disease, disorder, or condition in a subject by administering a compound of Formulas (I), (II) or (III) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides compositions for use in treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of (I), (II) or (III), or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formulas (I), (II) or (III) or a pharmaceutically acceptable salt, solvate, hydrate, tautomer, or stereoisomer thereof in a biological sample or subject. In another aspect, the present disclosure provides compositions for use in altering the isoform of a target protein with a compound of Formulas (I), (II) or (III) or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formulas (I), (II) or (III) to a biological sample, a cell, or a subject comprises inhibition of cell growth or induction of cell death.

In another aspect, the present disclosure features kits comprising a container with a compound of Formulas (I), (II) or (III), or a pharmaceutically acceptable salt, solvate, hydrate, tautomer, stereoisomer thereof, or a pharmaceutical composition thereof. In certain embodiments, the kits described herein further include instructions for administering the compound of Formulas (I), (II) or (III), or the pharmaceutically acceptable salt, solvate, hydrate, tautomer, stereoisomer thereof, or the pharmaceutical composition thereof.

In any and all aspects of the present disclosure, in some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein other than a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, and WO 2020/004594. In some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, and WO 2020/004594, each of which is incorporated herein by reference in its entirety.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—Cl₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 16-membered non-aromatic ring system having ring carbon atoms and 1 to 4 ring heteroatoms, wherein each heteroatom is independently selected from nitrogen, oxygen, sulfur, boron, phosphorus, and silicon (“3-16 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-16 membered heterocyclyl. In certain embodiments, the heterocyclyl group is substituted 3-16 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′— and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I) or (II) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and 14C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formulas (I), (II) or (III) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomer” refers to compounds that are interchangeable forms of a particular compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Another example of tautomerism is the aci- and nitro- forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formulas (I), (II) or (III) refers to an amount sufficient to elicit the desired biological response, i.e., treating the condition. As will be appreciated by those of ordinary skill in this art, the effective amount of a compound of Formulas (I) or (II) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formulas (I), (II) or (III) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formulas (I), (II) or (III)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formulas (I), (II) or (III)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

The present disclosure features a compound of Formula (I):

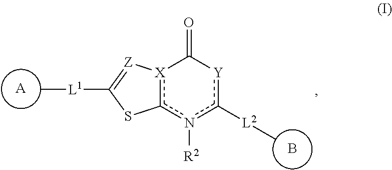

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ and L² are each independently absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; X is N or C; Y is N, N(R^{5a}), C(R^{5b}), or C(R^{5b})(R^{5c}), wherein the dashed lines representing bonds in the ring comprising X and Y may be single or double bonds as valency permits; Z is N or C(R⁶), wherein at least one of X, Y, and Z is independently N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, C₁-C₆ alkylene-aryl, C₂-C₆ alkenylene-aryl, C₁-C₆ alkylene-heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; R² is absent, hydrogen, or C₁-C₆-alkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R^{5a} is hydrogen, C₁-C₆-alkyl, C₁-C₆-heteroalkyl, C₁-C₆-haloalkyl; each of R^{5b} and R^{5c} is independently hydrogen, C₁-C₆-alkyl, C₁-C₆-heteroalkyl, C₁-C₆-haloalkyl, halo, or —OR^{A}; R⁶ is hydrogen, C₁-C₆-alkyl, C₁-C₆-heteroalkyl, C₁-C₆-haloalkyl, halo, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸; each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In another aspect, the present disclosure features a compound of Formula (II):

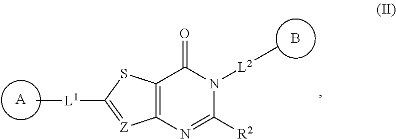

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ and L² are each independently absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; Z is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is absent, hydrogen, or C₁-C₆-alkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; R⁶ is hydrogen, C₁-C₆-alkyl, C₁-C₆-heteroalkyl, C₁-C₆-haloalkyl, halo, cyano, cycloalkyl, heterocyclyl, or —OR^{A}; R⁷ is hydrogen, C₁-C₆-alkyl, C₁-C₆-heteroalkyl, C₁-C₆-haloalkyl, halo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆-alkyl or halo; and x is 0, 1, or 2.

In another aspect, the present disclosure features a compound of Formula (III):

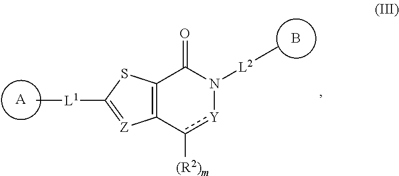

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ and L² are each independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; Y is N, N(R^{3a}), C(R^{3b}), or C(R^{3b})(R^{3c}), wherein the dashed lines representing bonds in the ring comprising Y may be single or double bonds as valency permits; Z is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —C(O)R^{D}, or —S(O)ₓR^{D}; R^{3b} and R^{3c} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D}; R⁴ is hydrogen, C₁-C₆-alkyl, or C₁-C₆-haloalkyl; R⁵ is C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁶ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸; each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; m is 1 or 2, wherein when the dashed lines representing bonds in the ring comprising Y are single bonds, m is 2, and when the dashed lines representing bonds in the ring comprising Y are double bonds, m is 1; and x is 0, 1, or 2.

As generally described herein for compounds of Formula(I), (II) and (III), each of A or B are independently cycloalkyl, heterocyclyl, aryl, or heteroaryl, each of which is optionally substituted with one or more R¹.

In some embodiments, each of A and B are independently a monocyclic ring, e.g., monocyclic cycloalkyl, monocyclic heterocyclyl, monocyclic aryl, or monocyclic heteroaryl.

The monocyclic ring may be saturated, partially unsaturated, or fully unsaturated (e.g., aromatic).

In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, A or B are independently a monocyclic ring optionally substituted with one or more R¹.

In some embodiments, A or B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, A or B are independently a bicyclic ring optionally substituted with one or more R¹.

In some embodiments, A or B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, A or B are independently a tricyclic ring optionally substituted with one or more R¹.

In some embodiments, A or B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl.

In some embodiments, A or B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹.

In some embodiments, A or B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹. In some embodiments, B is a 5-membered nitrogen-containing heteroaryl optionally substituted with one or more R¹. In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

In some embodiments, B is a nitrogen-containing bicyclic heteroaryl (e.g., a 9-membered nitrogen-containing bicyclic heteroaryl), that is optionally substituted with one or more R¹. In some embodiments, B is a 9-membered bicyclic heteroaryl comprising 1 nitrogen atom. In some embodiments, B is a 9-membered bicyclic heteroaryl comprising 2 nitrogen atoms. In some embodiments, B is a 9-membered bicyclic heteroaryl comprising 3 nitrogen atoms. In some embodiments, B is a 9-membered bicyclic heteroaryl comprising 4 nitrogen atoms. The one or more nitrogen atom of the 9-membered bicyclic heteroaryl may be at any position of the ring. In some embodiments, B is a 9-membered bicyclic heteroaryl substituted with one or more R¹.

In some embodiments, each of A and B are independently selected from:

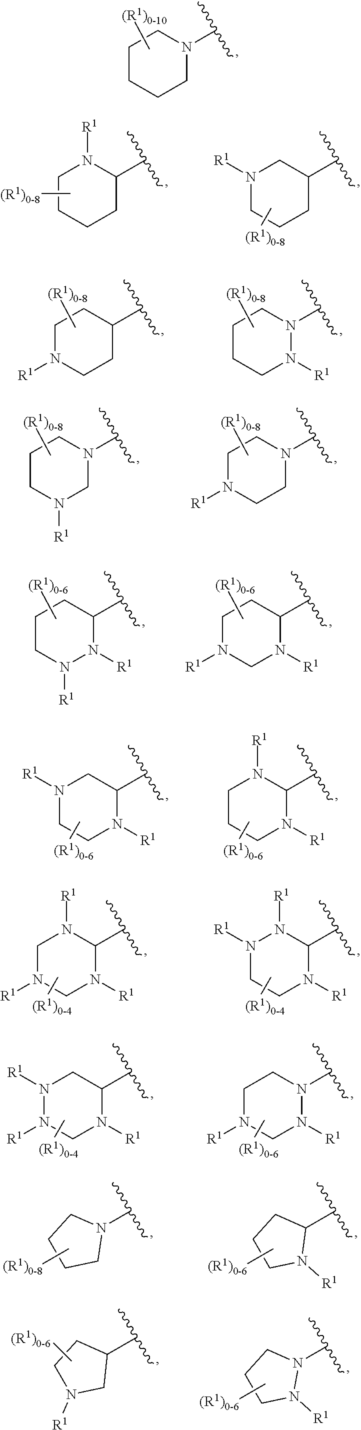

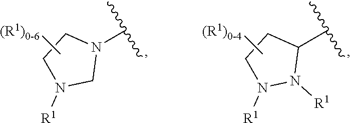

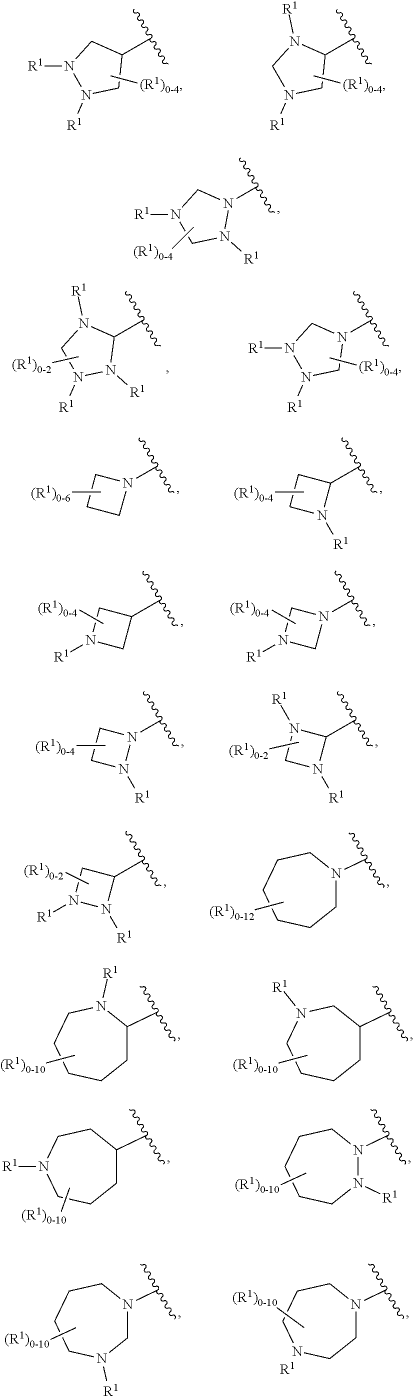

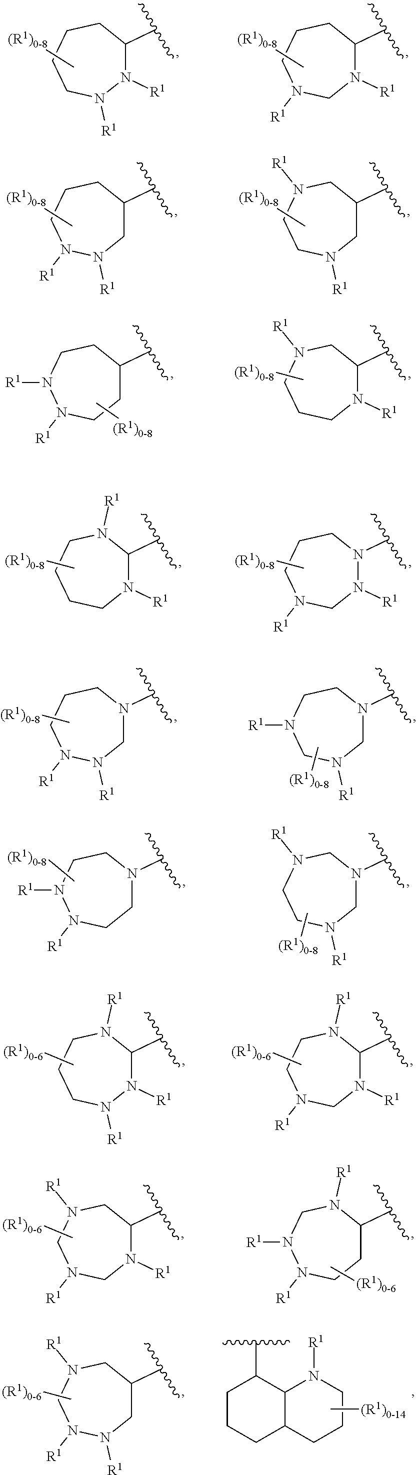

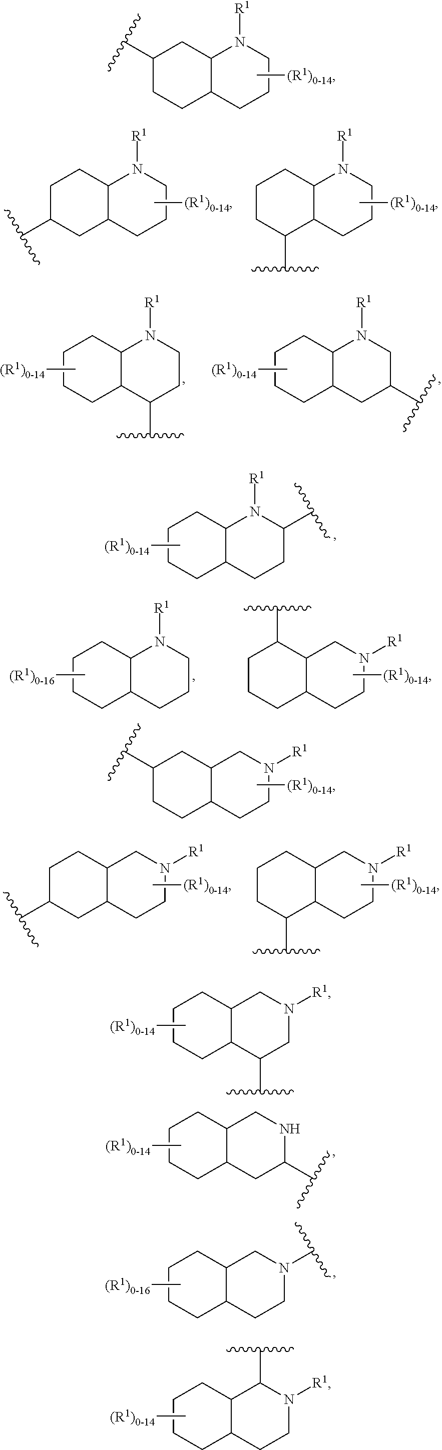

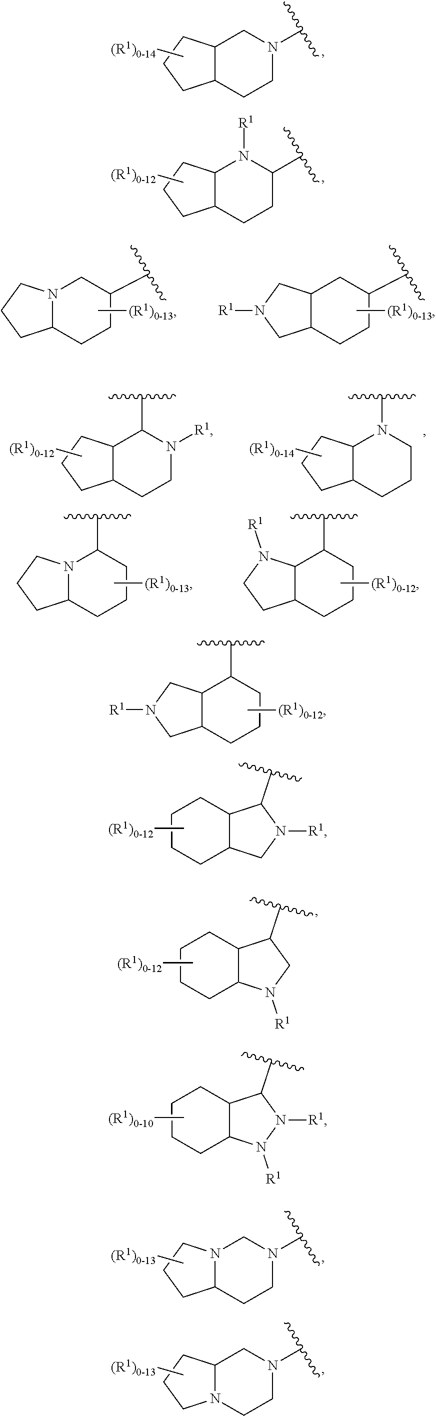

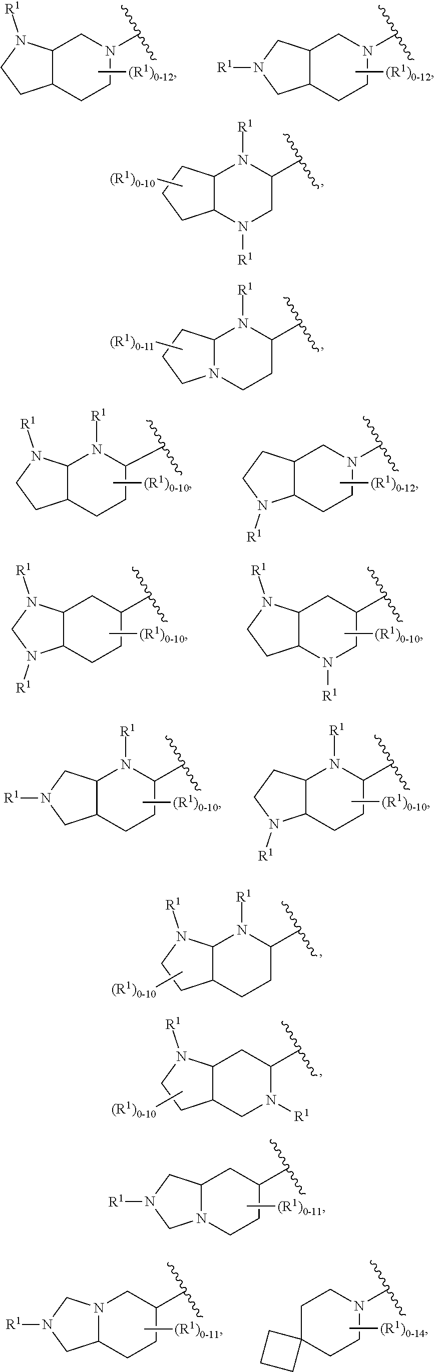

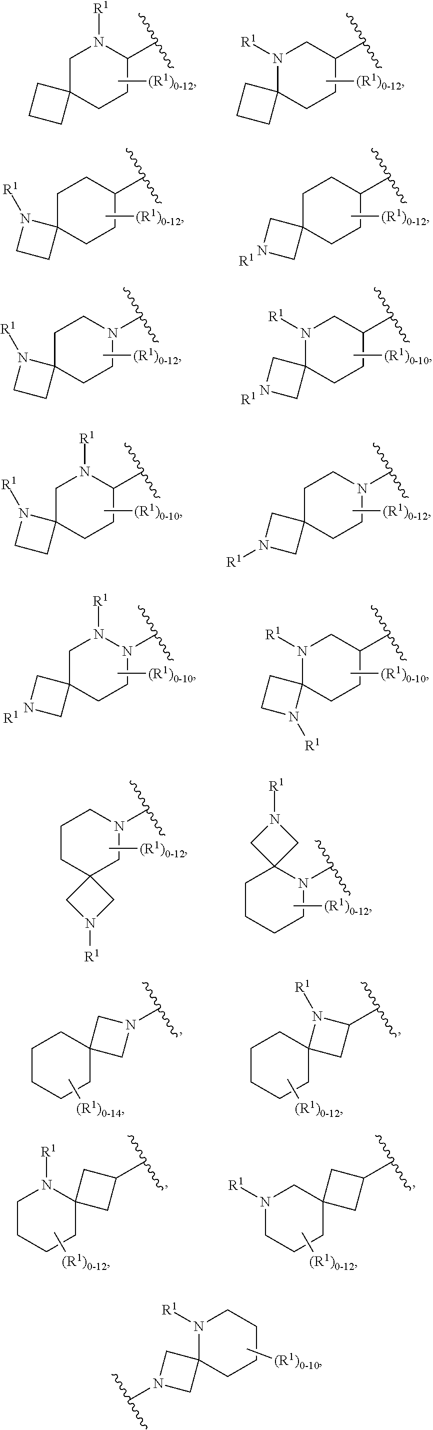

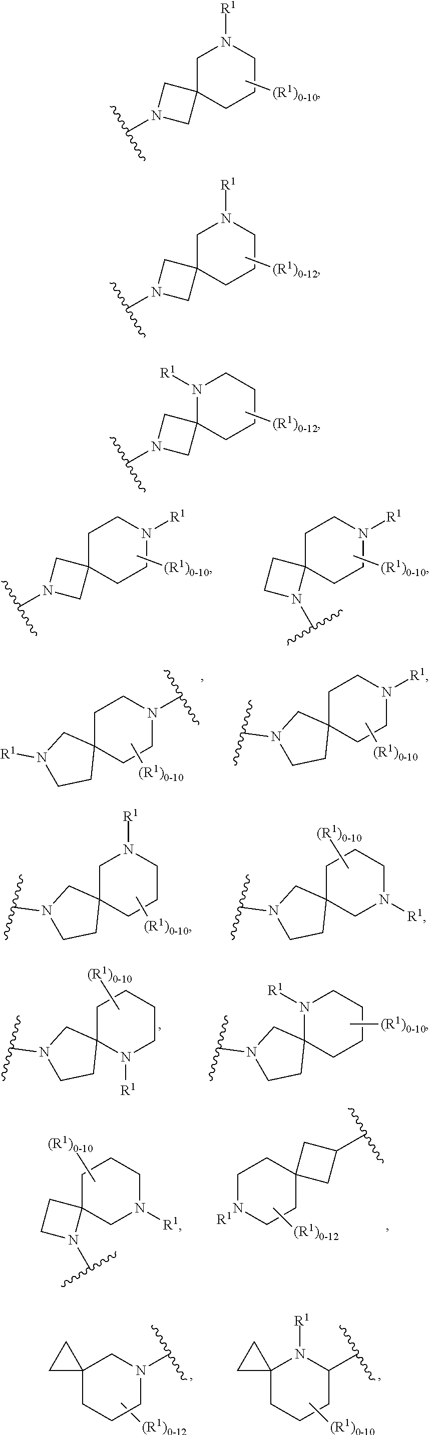

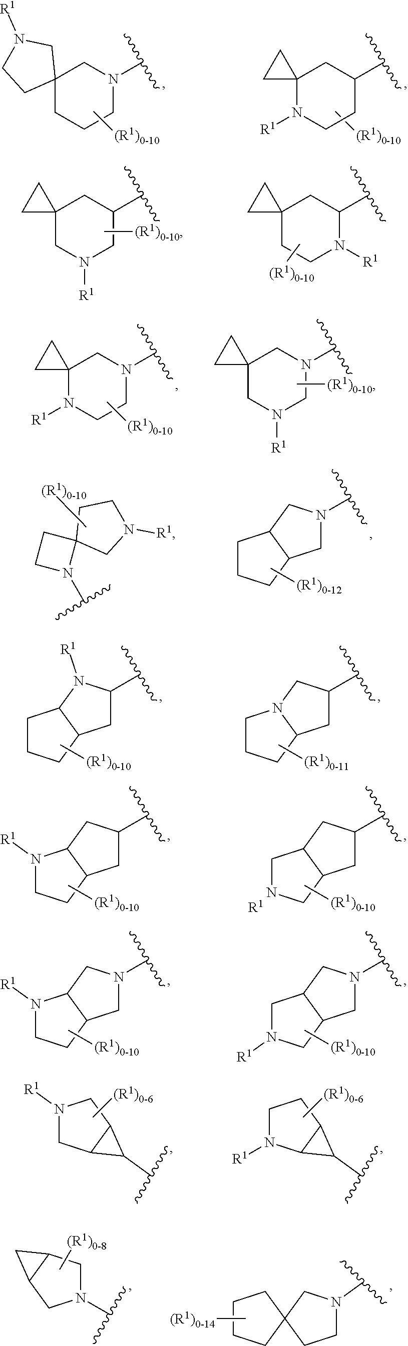

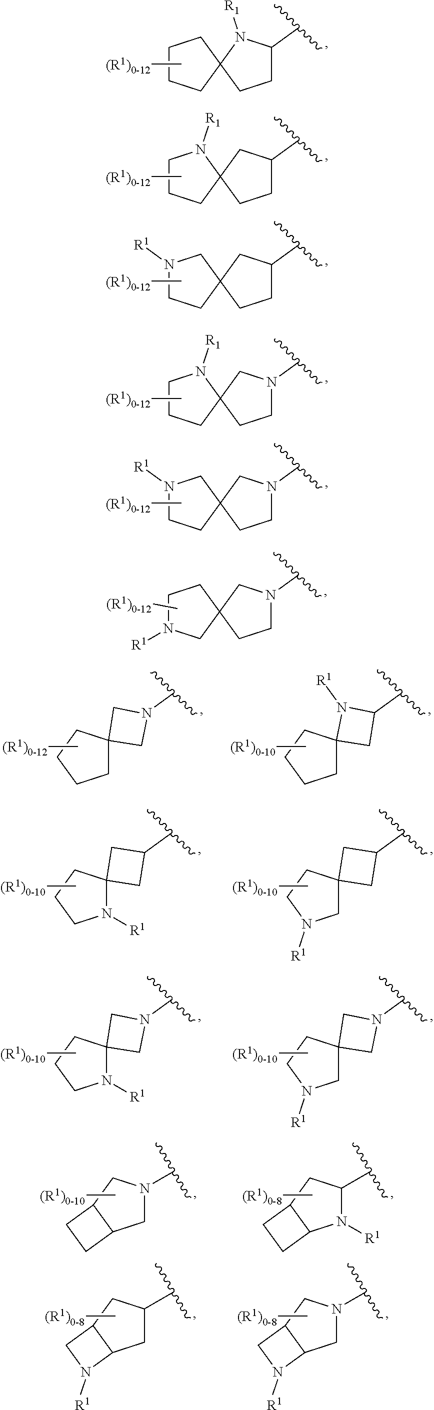

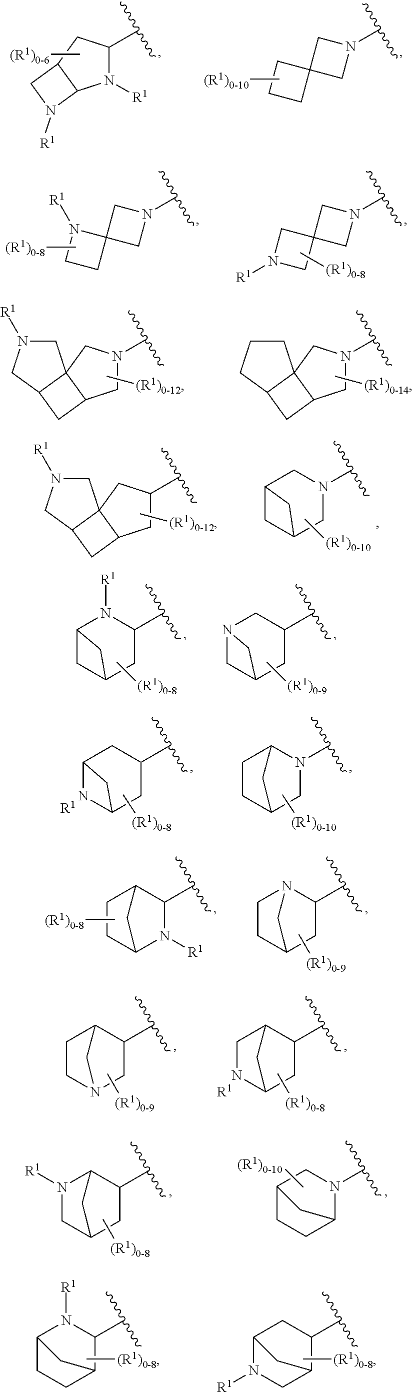

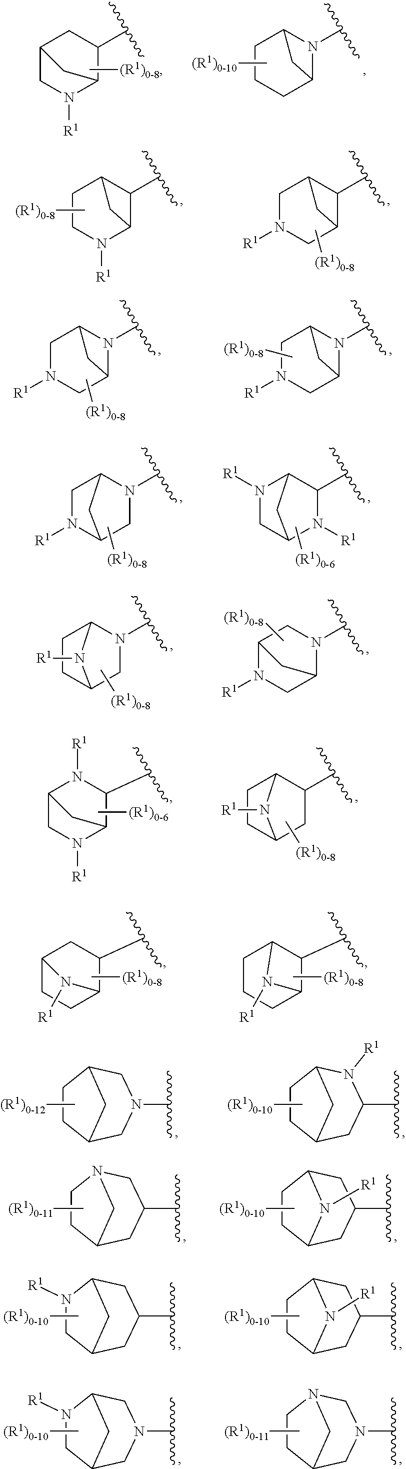

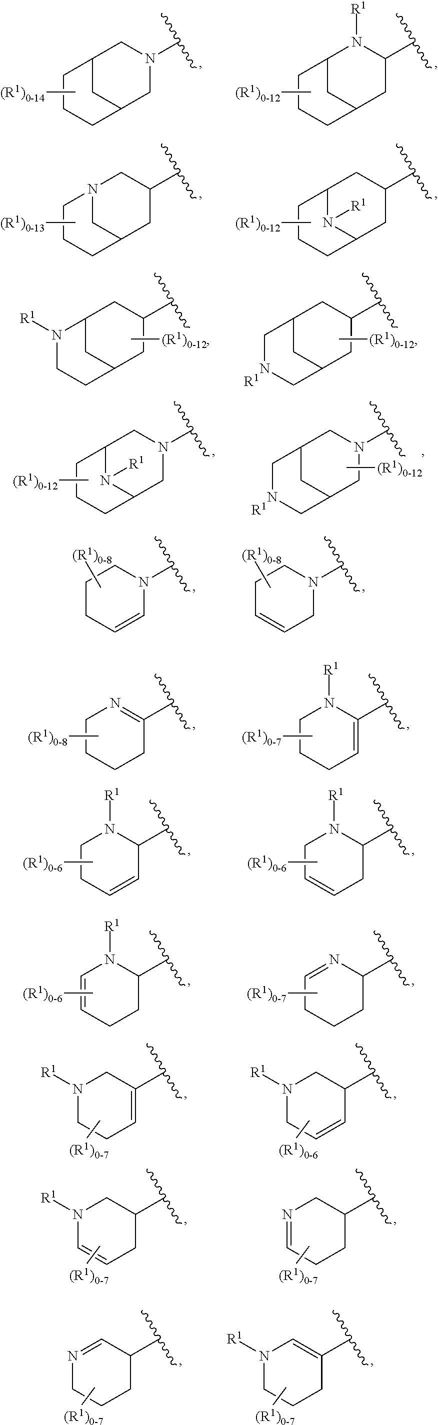

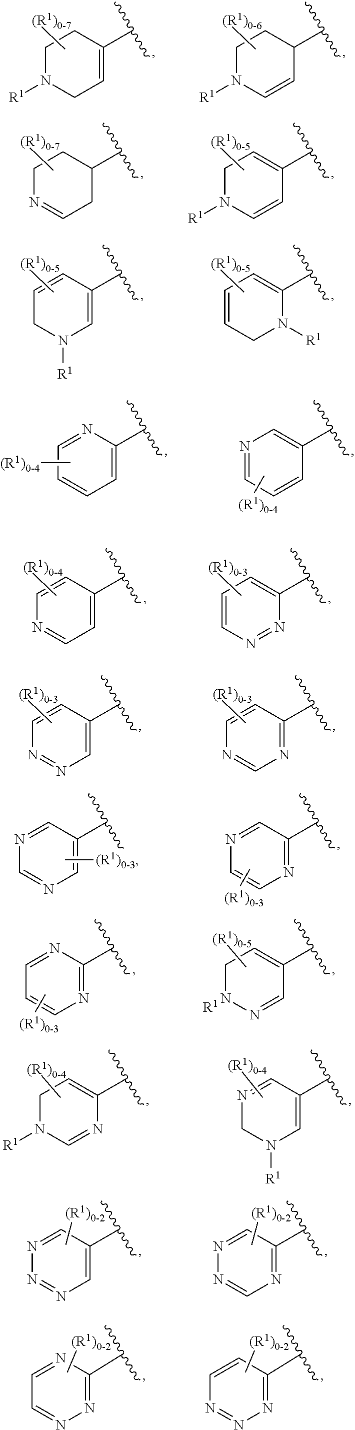

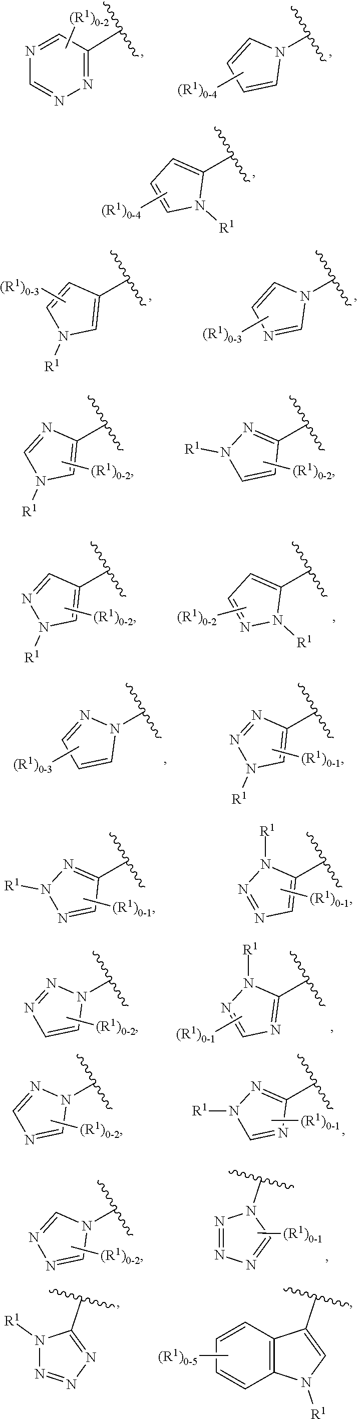

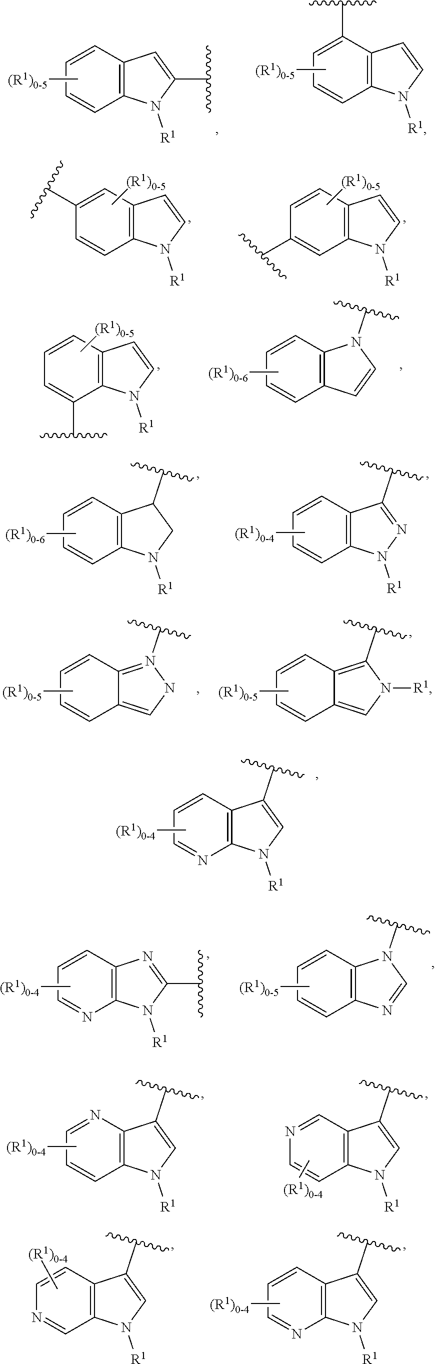

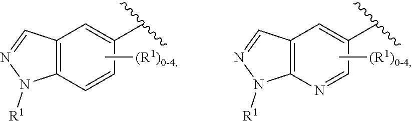

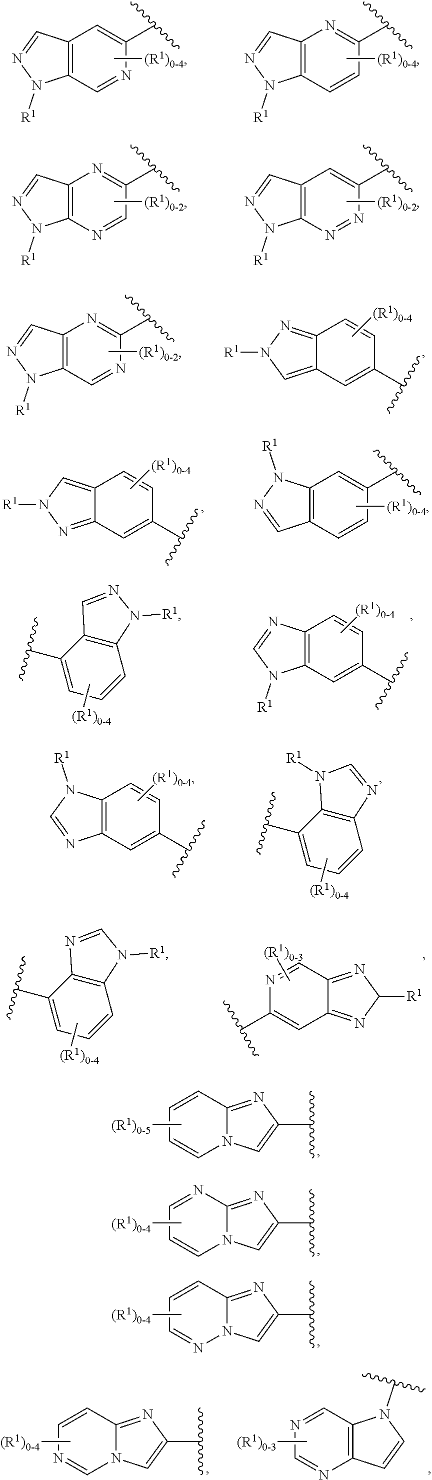

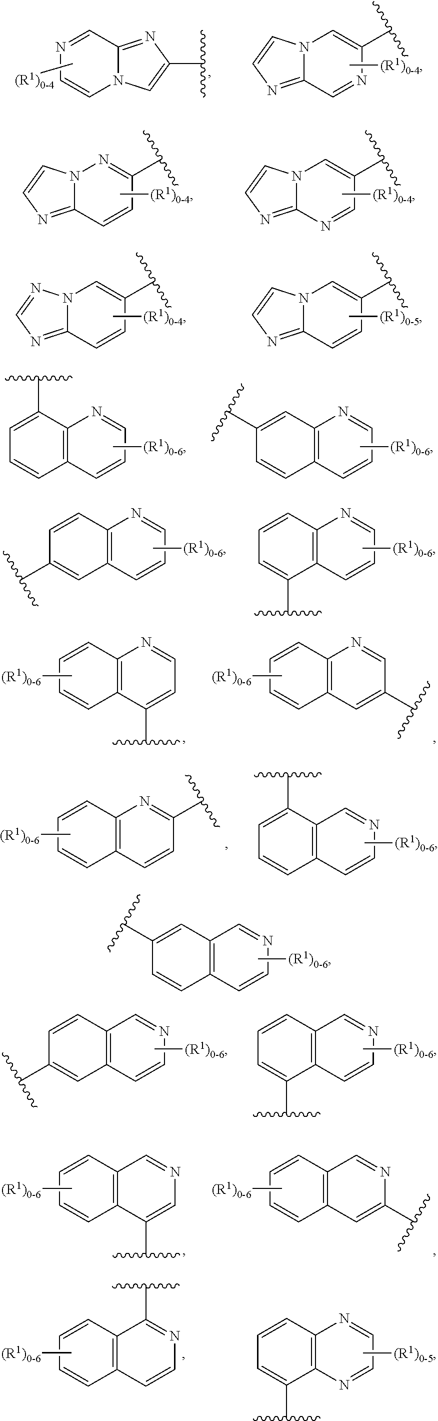

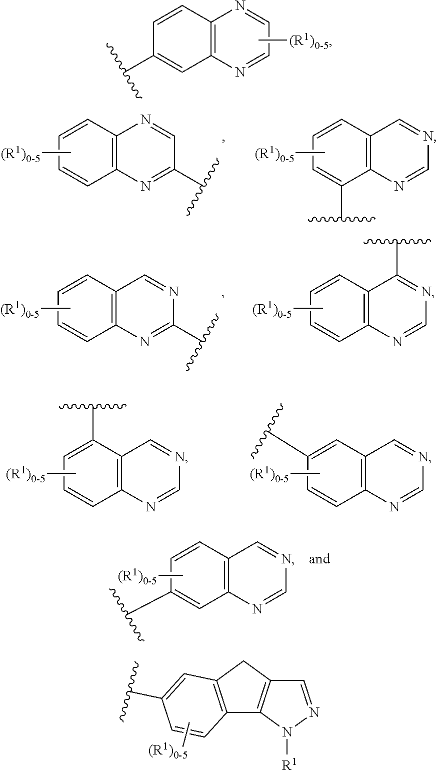

wherein each R is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

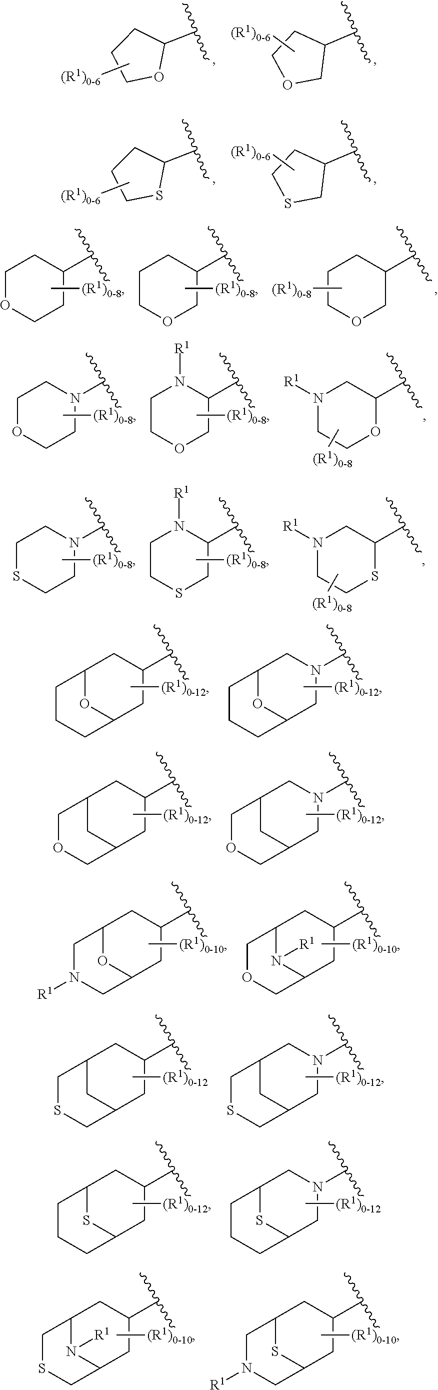

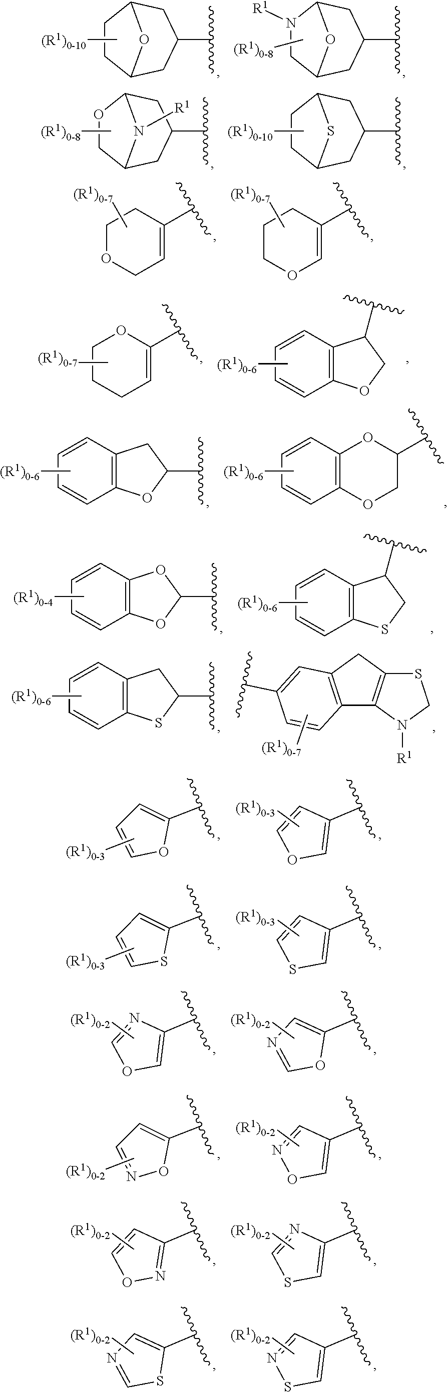

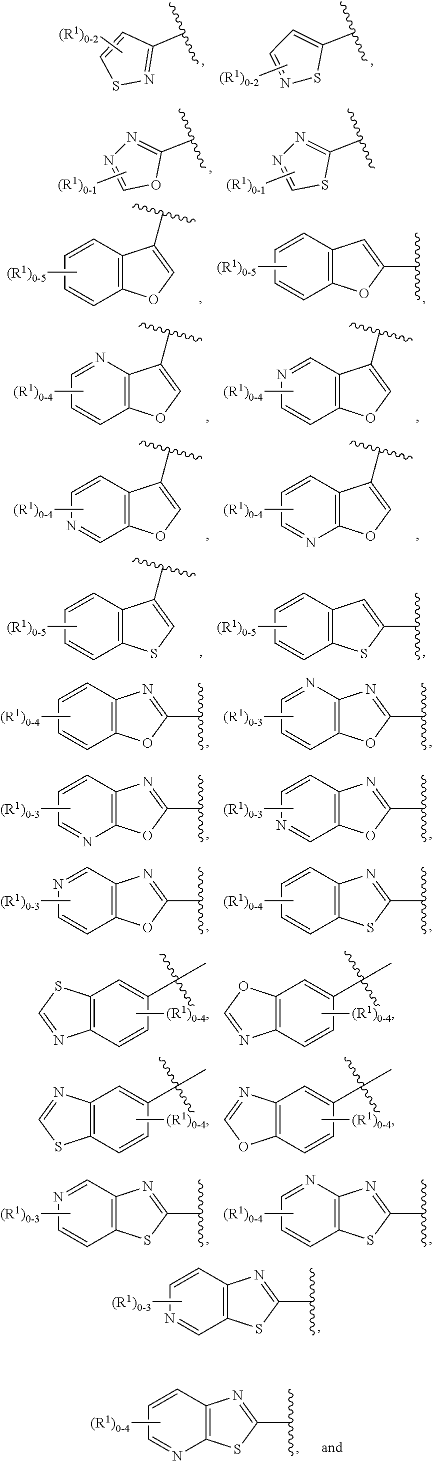

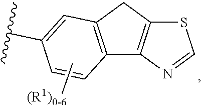

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, A is selected from

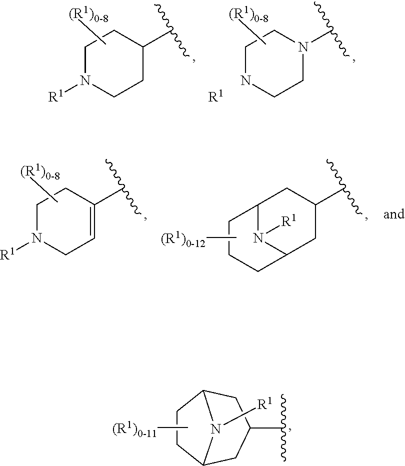

wherein R¹ is as defined herein.

In some embodiments, A is selected from

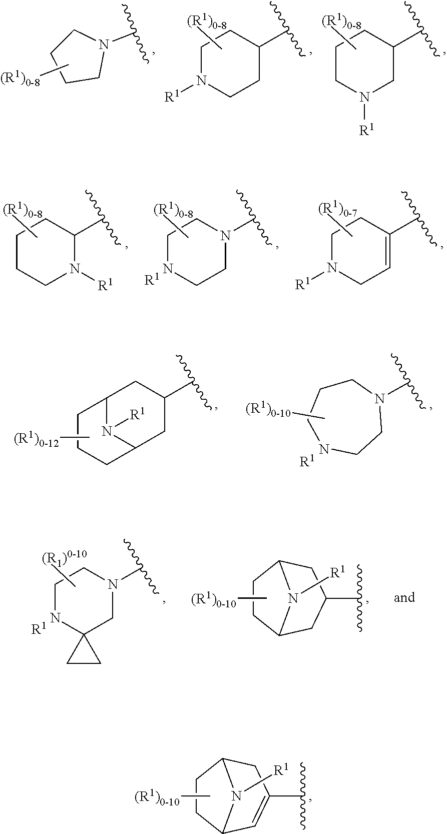

wherein R¹ is as defined herein. In some embodiments, A is selected from

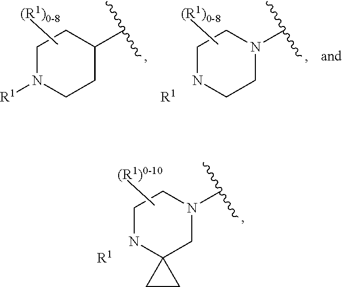

wherein R¹ is as defined herein. In some embodiments, A is selected from

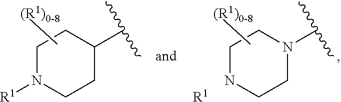

wherein R¹ is as defined herein.

In some embodiments, A is selected from

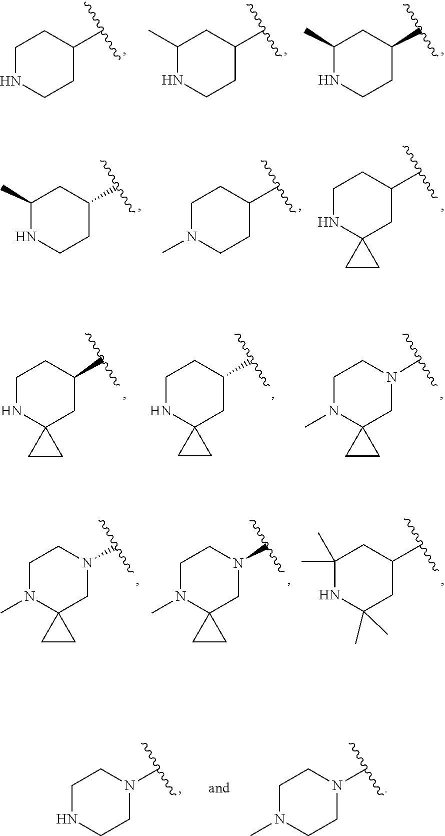

In some embodiments, A is selected from

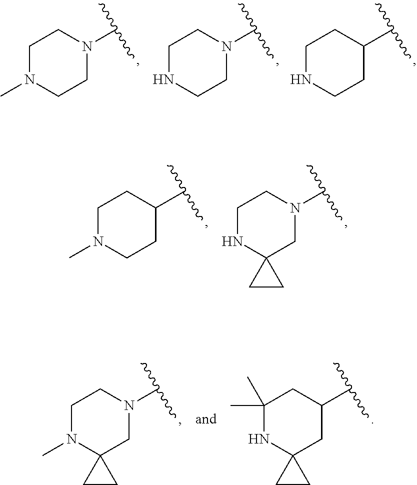

In some embodiments, A is heteroaryl optionally substituted with one or more R¹. In some embodiments, A is bicyclic nitrogen-containing heteroaryl. In some embodiments, A is selected from

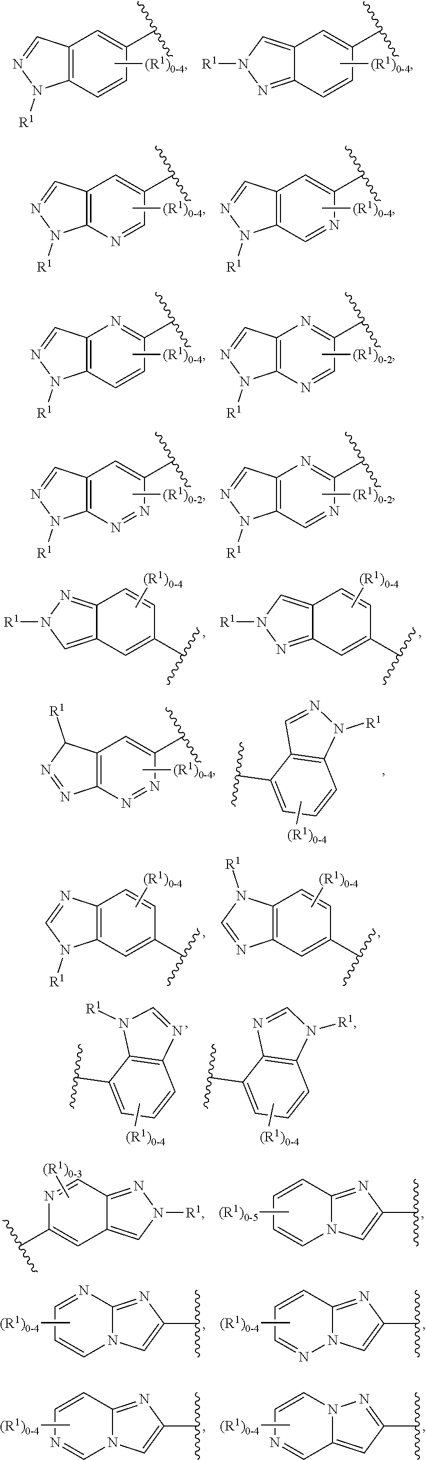

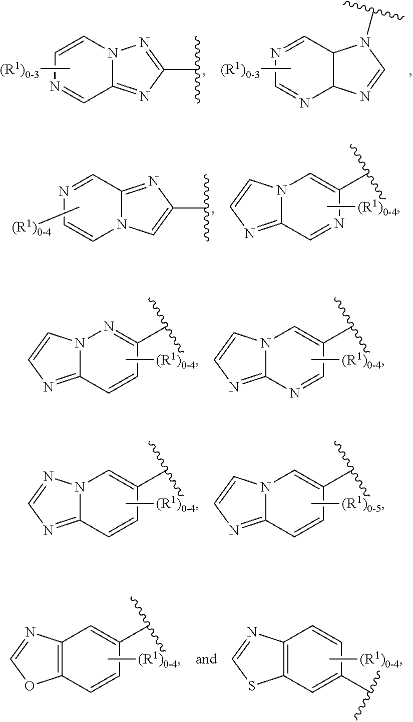

wherein R¹ is as defined herein. In some embodiments, A is selected from

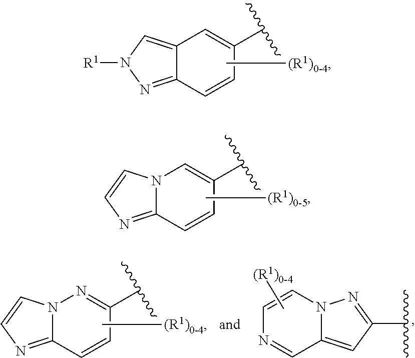

wherein R¹ is as described herein.

In some embodiments, A is selected from

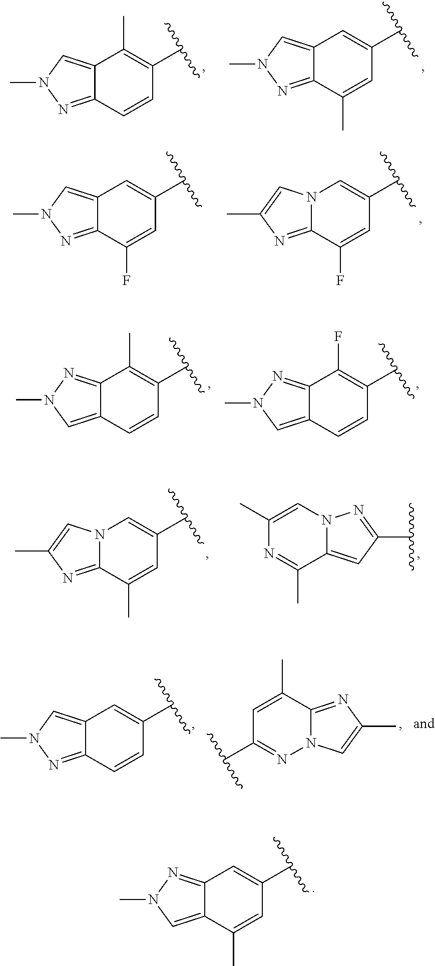

In some embodiments, A is selected from

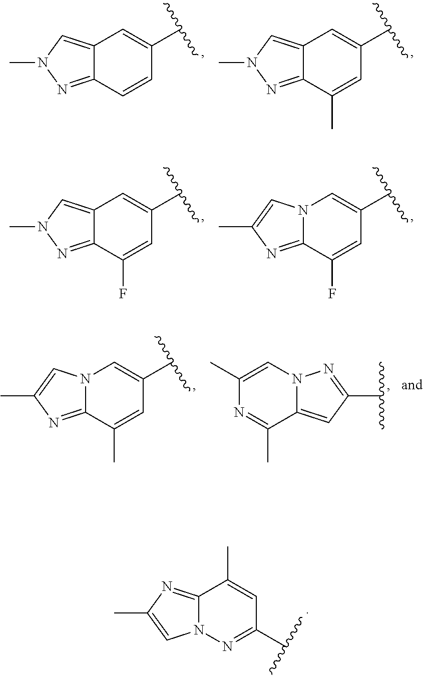

In some embodiments, A is

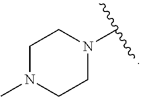 [SMILES: *N1CCN(C)CC1]

In some embodiments, A is

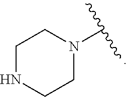 [SMILES: *N1CCNCC1]

In some embodiments, A is

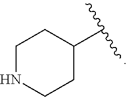 [SMILES: *C1CCNCC1]

In some embodiments, A is

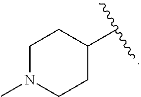 [SMILES: *C1CCN(C)CC1]

In some embodiments, A is

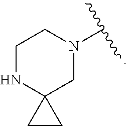 [SMILES: *N1CCNC2(CC2)C1]

In some embodiments, A is

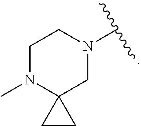 [SMILES: *N1CCN(C)C2(CC2)C1]

In some embodiments, A is

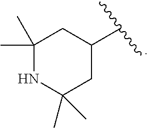 [SMILES: *C1CC(C)(C)NC(C)(C)C1]

In some embodiments, A is

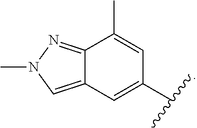 [SMILES: *c1cc(C)c2nn(C)cc2c1]

In some embodiments, A is

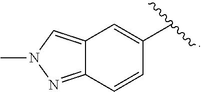 [SMILES: *c1ccc2nn(C)cc2c1]

In some embodiments, A is

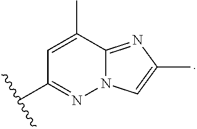 [SMILES: *c1cc(C)c2nc(C)cn2n1]

In some embodiments A is

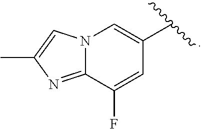 [SMILES: *c1cc(F)c2nc(C)cn2c1]

In some embodiments, B is selected from

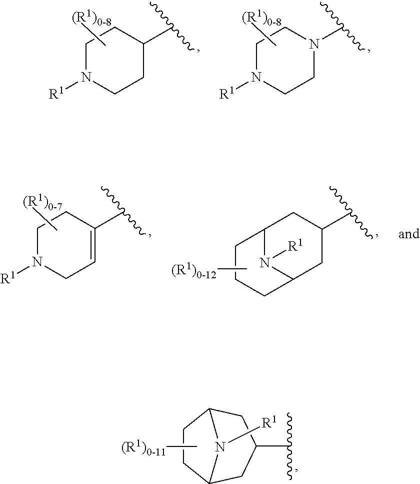

wherein R¹ is as defined herein.

In some embodiments, B is selected from

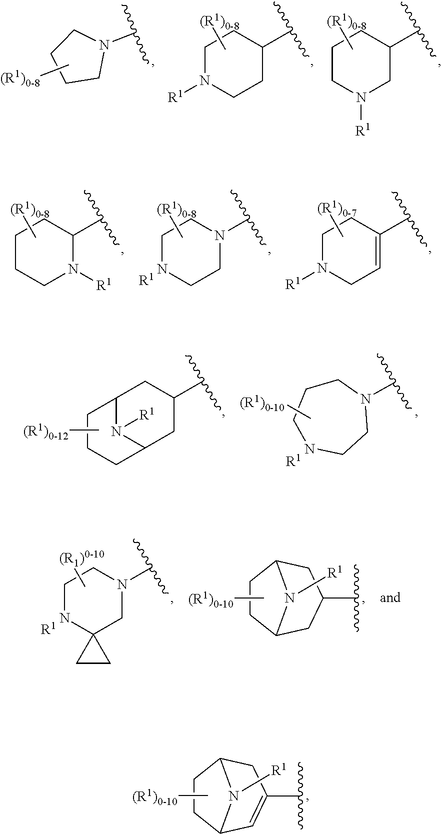

wherein R¹ is as defined herein. In some embodiments, B is selected from

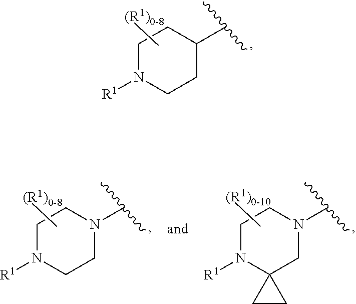

wherein R¹ is as defined herein. In some embodiments, B is selcted from

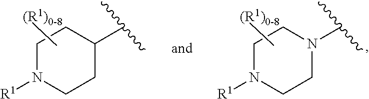

wherein R¹ is as defined herein.

In some embodiments, B is selected from

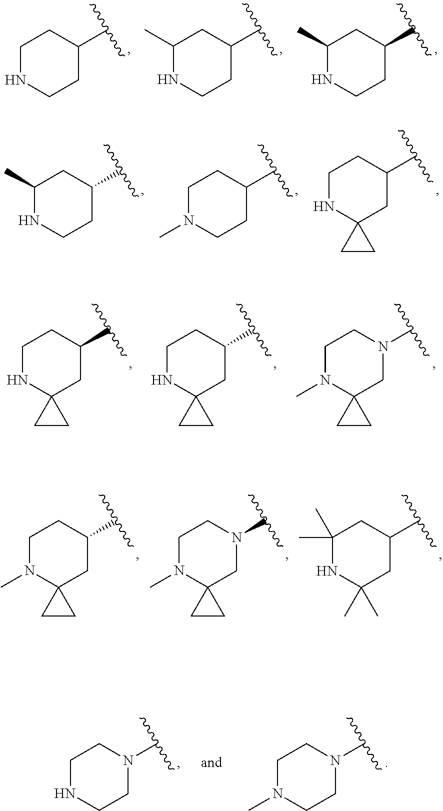

In some embodiments, A is selected from

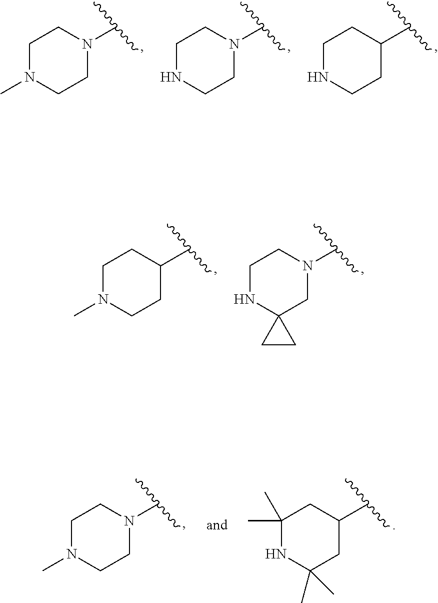

In some embodiments, B is heteroaryl optionally substituted with one or more R. In some embodiments, A is bicyclic nitrogen-containing heteroaryl. In some embodiments, B is selected from

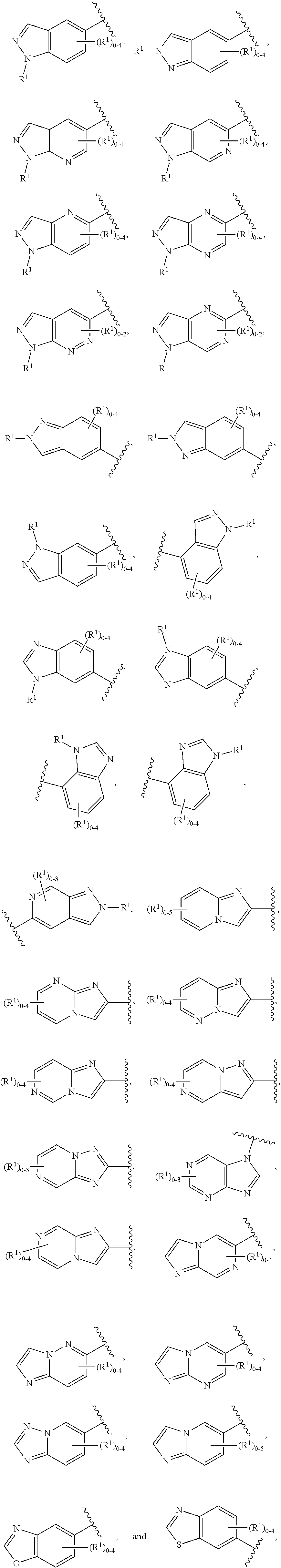

wherein R¹ is as defined herein. In some embodiments, B is selected from

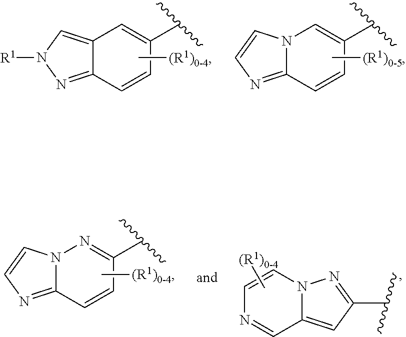

wherein R¹ is as described herein.

In some embodiments, B is selected from

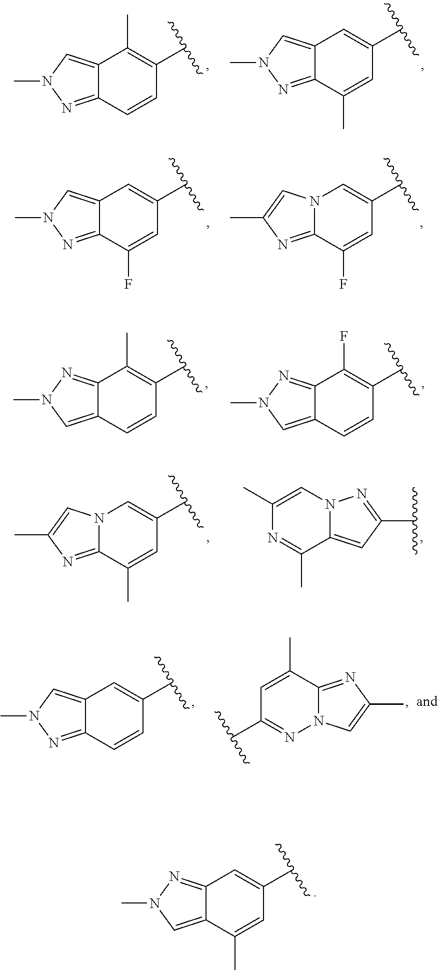

In some embodiments, B is selected from

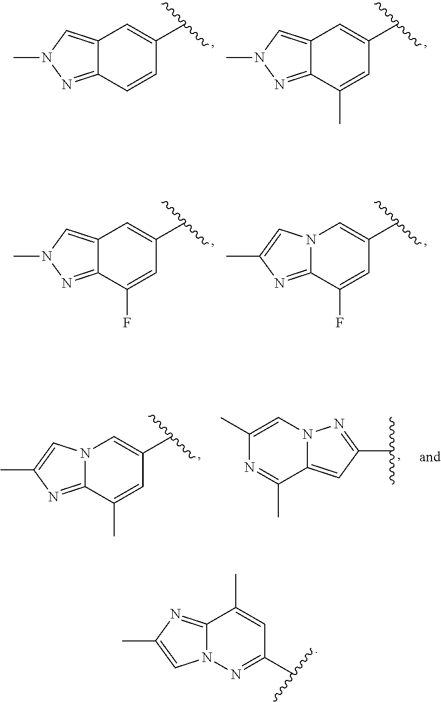

In some embodiments, B is

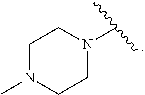 [SMILES: *N1CCN(C)CC1]

In some embodiments, B is

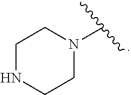 [SMILES: *N1CCNCC1]

In some embodiments, B is

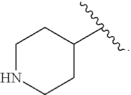 [SMILES: *C1CCNCC1]

In some embodiments, B is

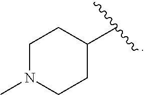 [SMILES: *C1CCN(C)CC1]

In some embodiments, B is

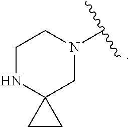 [SMILES: *N1CCNC2(CC2)C1]

In some embodiments, B is

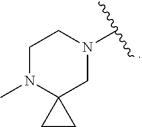 [SMILES: *N1CCN(C)C2(CC2)C1]

In some embodiments, B is

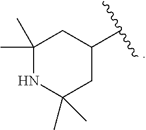 [SMILES: *C1CC(C)(C)NC(C)(C)C1]

In some embodiments, B is

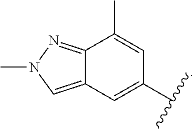 [SMILES: *c1cc(C)c2nn(C)cc2c1]

In some embodiments, B is

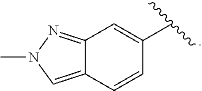 [SMILES: *c1ccc2cn(C)nc2c1]

In some embodiments, B is

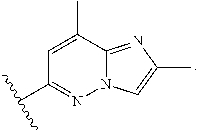 [SMILES: *c1cc(C)c2nc(C)cn2n1]

In some embodiments, B is

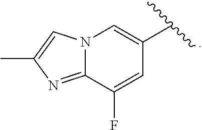 [SMILES: *c1cc(F)c2nc(C)cn2c1]

As generally described herein for Formulas (I), (II) and (III), each of L¹ and L² may be absent or refer to a C₁-C₆-alkylene, C₁-C₆-heteroalkylene, —O—, —C(O)—, —N(R³)—, —N(R³)C(O)—, or —C(O)N(R³)— group, wherein each alkylene and heteroalkylene is optionally substituted with one or more R⁴. In some embodiments, each of L¹ and L² is independently absent or C₁-C₆-heteroalkylene. In some embodiments, each of L¹ and L² is independently absent. In some embodiments, each of L¹ and L² is independently C₁-C₆-heteroalkylene (e.g., —N(CH₃)—). In some embodiments, L1 and L2 are absent or C₁-C₆-heteroalkylene (e.g., —N(CH₃)—). In some embodiments, L1 and L2 are absent. In some embodiments, L1 and L2 are C₁-C₆-heteroalkylene (e.g., —N(CH₃)—). In some embodiments, L² is absent or C₁-C₆-heteroalkylene (e.g., —N(CH₃)—). In some embodiments, L² is absent. In some embodiments, L² is C₁-C₆-heteroalkylene (e.g., —N(CH₃)—).

As generally described herein for Formula (I), X may be N or C. In some embodiments, X is N. In some embodiments, X is C.

As generally described herein for Formula (I), Y may be N, N(R^{5a}), C(R^{5b}), or C(R^{5b})(R^{5c}), wherein the bonds in the ring comprising X and Y may be single or double bonds as valency permits. In some embodiments, Y is N(R^{5a}) or C(R^{5b}). In some embodiments, Y is N(R^{5a}) (e.g., NH). In some embodiments, Y is C(R^{5b}) (e.g., CH).

In some embodiments, X is C and Y is N(R^{5a}). In some embodiments, X is C and Y is NH. In some embodiments, X is N and Y is C(R^{5b}). In some embodiments, X is N and Y is CH.

As generally described herein for Formula (II), Y may be N, C, or C(R^{5b}), wherein the bonds in the ring comprising X and Y may be single or double bonds as valency permits. In some embodiments, Y is N. In some embodiments, Y is C. In some embodiment, Y is C(R^{5b}) (e.g. CH).

As generally described herein for Formulas (I), (II), (III), and (IV), Z may be N or C(R⁶). In some embodiments, Z is N. In some embodiments, Z is C(R⁶) (e.g., CH).

In some embodiments, X is C and Z is N. In some embodiments, X is N and Z is N. In some embodiments, X is N and Z is C(R⁶). In some embodiments, X is N and Z is CH. In some embodiments, Y is N(R^{5a}) and Z is N. In some embodiments, Y is NH and Z is N. In some embodiments, Y is C(R^{5b}) and Z is N. In some embodiments, Y is CH and Z is N. In some embodiments, Y is C(R^{5b}) and Z is C(R⁶). In some embodiments, Y is CH and Z is CH.

In some embodiments, X is C, Y is N(R^{5a}), and Z is N. In some embodiments, X is C, Y is NH and Z is N. In some embodiments, X is N, Y is C(R^{5b}), and Z is N. In some embodiments, X is N, Y is CH and Z is N. In some embodiments, X is N, Y is C(R^{5b}), and Z is C(R⁶). In some embodiments, X is N, Y is CH and Z is CH. In some embodiments, R² is absent.

In some embodiments, R¹ is C₁-C₆-alkyl. In some embodiments, R¹ is CH₃. In some embodiments, A is substituted with 0 or 1 R¹. In some embodiments, B is substituted with 0, 1, or 2 R¹.

In some embodiments, the compound of Formula (I) is a compound of Formula (I-a):

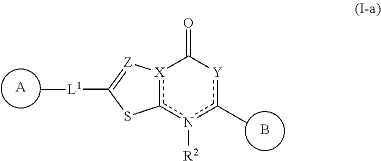

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; X is N or C; Y is N, N(R^{5a}), C(R^{5b}), or C(R^{5b})(R^{5c}), wherein the dashed lines representing bonds in the ring comprising X and Y may be single or double bonds as valency permits; Z is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; R² is absent, hydrogen, or C₁-C₆-alkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R^{5a} is hydrogen, C₁-C₆-alkyl, C₁-C₆-heteroalkyl, C₁-C₆-haloalkyl; each of R^{5b} and R^{5c} is independently hydrogen, C₁-C₆-alkyl, C₁-C₆-heteroalkyl, C₁-C₆-haloalkyl, halo, or —OR^{A}; R⁶ is hydrogen, C₁-C₆-alkyl, C₁-C₆-heteroalkyl, C₁-C₆-haloalkyl, or halo; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸; each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

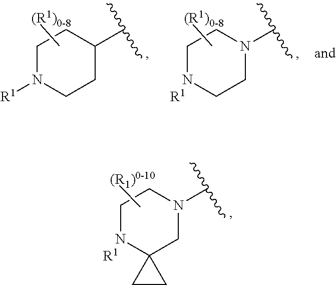

wherein R¹ is as defined herein.

In some embodiments, A is selected from

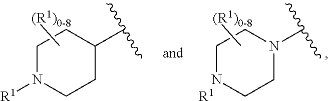

wherein R¹ is as defined herein.

In some embodiments, A is selected from

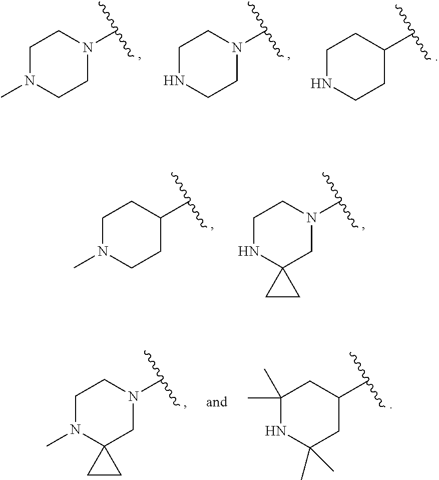

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

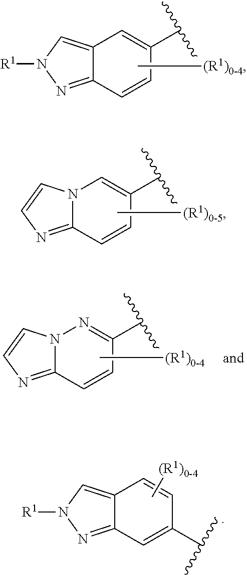

In some embodiments, A is

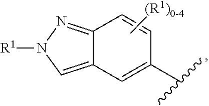

wherein R¹ is as defined herein.

In some embodiments, A is selected from

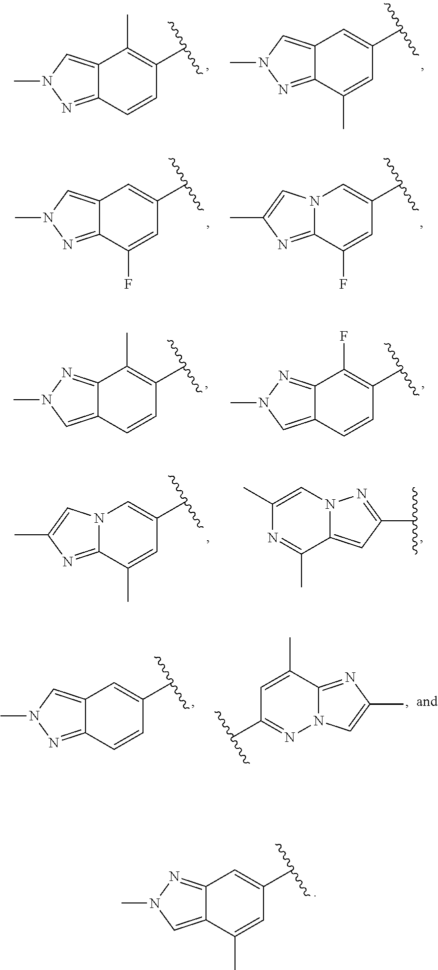

In some embodiments, A is

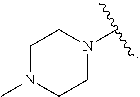 [SMILES: *N1CCN(C)CC1]

In some embodiments, A is

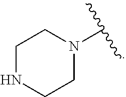 [SMILES: *N1CCNCC1]

In some embodiments, A is

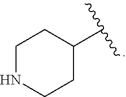 [SMILES: *C1CCNCC1]

In some embodiments, A is

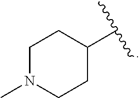 [SMILES: *C1CCN(C)CC1]

In some embodiments, A is

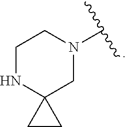 [SMILES: *N1CCNC2(CC2)C1]

In some embodiments, A is

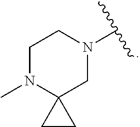 [SMILES: *N1CCN(C)C2(CC2)C1]

In some embodiments, A is

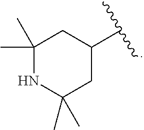 [SMILES: *C1CC(C)(C)NC(C)(C)C1]

In some embodiments, A is

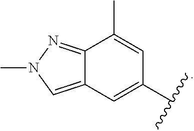 [SMILES: *c1cc(C)c2nn(C)cc2c1]

In some embodiments, A is

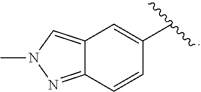 [SMILES: *c1ccc2nn(C)cc2c1]

In some embodiments, A is

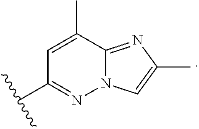 [SMILES: *c1cc(C)c2nc(C)cn2n1]

In some embodiments, A is

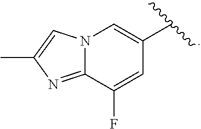 [SMILES: *c1cc(F)c2nc(C)cn2c1]

In some embodiments, B is selected from

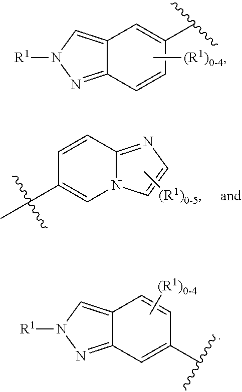

In some embodiments, B is selected from

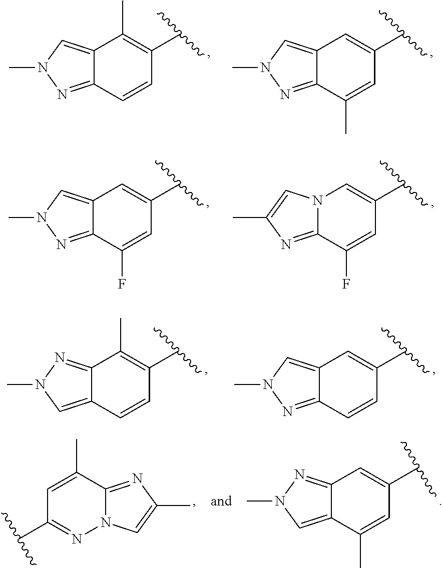

In some embodiments, B is heterocyclyl optionally substituted with one or more R¹. In some embodiments, B is monocyclic nitrogen-containing heterocyclyl. In some embodiments, B is selected from

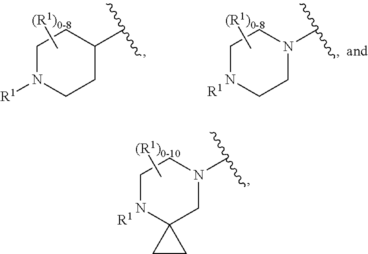

wherein R¹ is as defined herein.

In some embodiments, B is selected from

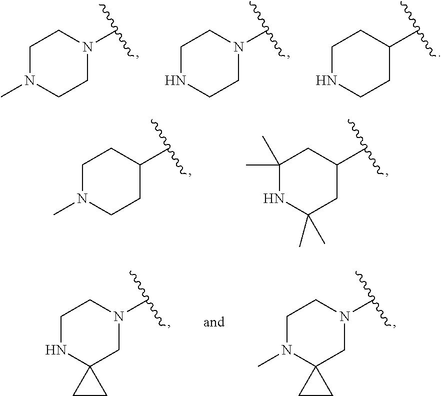

In some embodiments, B is

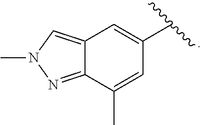 [SMILES: *c1cc(C)c2nn(C)cc2c1]

In some embodiments, B is

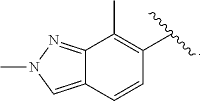 [SMILES: *c1ccc2cn(C)nc2c1C]

In some embodiments, B is

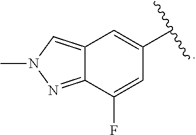 [SMILES: *c1cc(F)c2nn(C)cc2c1]

In some embodiments, B is

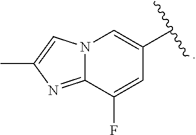 [SMILES: *c1cc(F)c2nc(C)cn2c1]

In some embodiments, B is

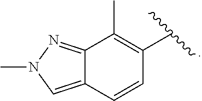 [SMILES: *c1ccc2cn(C)nc2c1C]

In some embodiments, B is

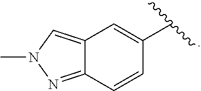 [SMILES: *c1ccc2nn(C)cc2c1]

In some embodiments, B is

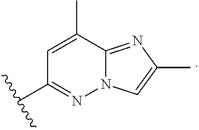 [SMILES: *c1cc(C)c2nc(C)cn2n1]

In some embodiments, B is

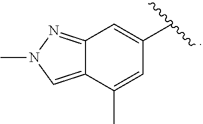 [SMILES: *c1cc(C)c2cn(C)nc2c1]

In some embodiments, B is

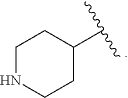 [SMILES: *C1CCNCC1]

In some embodiments, B

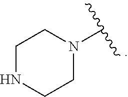 [SMILES: *N1CCNCC1]

In some embodiments, B is

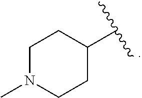 [SMILES: *C1CCN(C)CC1]

In some embodiments, B is

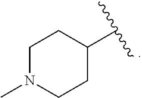 [SMILES: *C1CCN(C)CC1]

In some embodiments, B is

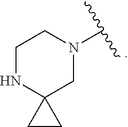 [SMILES: *N1CCNC2(CC2)C1]

In some embodiments, B is

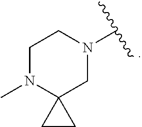 [SMILES: *N1CCN(C)C2(CC2)C1]

In some embodiments, B is

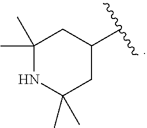 [SMILES: *C1CC(C)(C)NC(C)(C)C1]

In some embodiments, L¹ is absent or C₁-C₆-heteroalkylene (e.g., —N(CH₃)—). In some embodiments, L¹ is absent. In some embodiments, L¹ is C₁-C₆-heteroalkylene (e.g., —N(CH₃)—).

In some embodiments, X is N. In some embodiments, X is C.

In some embodiments, Y is N(R^{5a}) or C(R^{5b}). In some embodiments, Y is N(R^{5a}) (e.g., NH). In some embodiments, Y is C(R^{5b}) (e.g., CH).

In some embodiments, X is C and Y is N(R^{5a}). In some embodiments, X is C and Y is NH. In some embodiments, X is N and Y is C(R^{5b}). In some embodiments, X is N and Y is CH.

In some embodiments, Y is N. In some embodiments, Y is C. In some embodiment, Y is C(R^{5b}) (e.g. CH).

In some embodiments, Z is N. In some embodiments, Z is C(R⁶) (e.g., CH).

In some embodiments, the compound of Formula (I) is a compound of Formula (I-b):

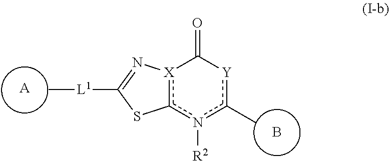

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; X is N or C; Y is N, N(R^{5a}), C(R^{5b}), or C(R^{5b})(R^{5c}), wherein the dashed lines representing bonds in the ring comprising X and Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; R² is absent, hydrogen, or C₁-C₆-alkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁵, is hydrogen, C₁-C₆-alkyl, C₁-C₆-heteroalkyl, C₁-C₆-haloalkyl; each of R^{5b} and R^{5c} is independently hydrogen, C₁-C₆-alkyl, C₁-C₆-heteroalkyl, C₁-C₆-haloalkyl, halo, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸; each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is optionally substituted piperazinyl. In some embodiments, A is selected from

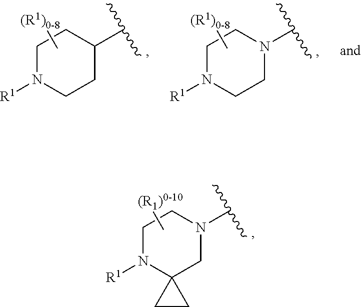

wherein R¹ is as defined herein.

In some embodiments, A is selected from

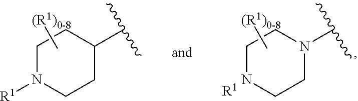

wherein R¹ is ad defined herein.

In some embodiments, A is selected from

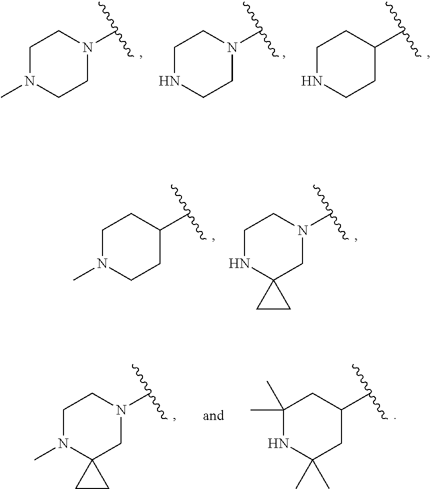

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

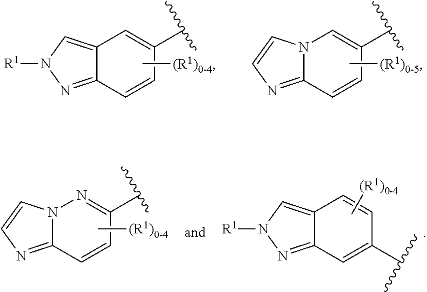

In some embodiments A is

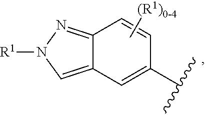

wherein R¹ is as defined herein.

In some embodiments, A is selected from

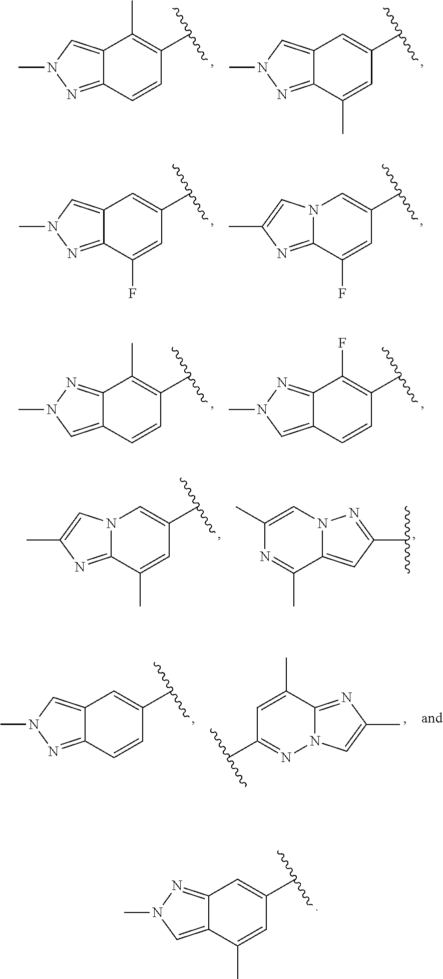

In some embodiments, A is

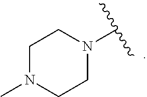 [SMILES: *N1CCN(C)CC1]

In some embodiments, A is

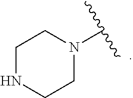 [SMILES: *N1CCNCC1]

In some embodiments, A is

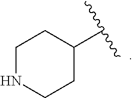 [SMILES: *C1CCNCC1]

In some embodiments, A is

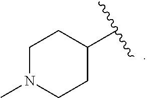 [SMILES: *C1CCN(C)CC1]

In some embodiments, A is

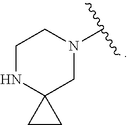 [SMILES: *N1CCNC2(CC2)C1]

In some embodiments, A is

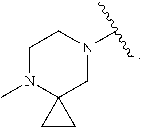 [SMILES: *N1CCN(C)C2(CC2)C1]

In some embodiments, A is

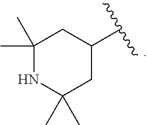 [SMILES: *C1CC(C)(C)NC(C)(C)C1]

In some embodiments, A is

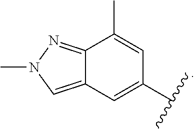 [SMILES: *c1cc(C)c2nn(C)cc2c1]

In some embodiments, A is

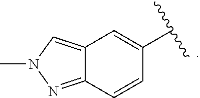 [SMILES: *c1ccc2nn(C)cc2c1]

In some embodiments, A is

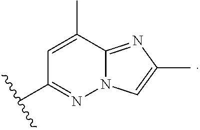 [SMILES: *c1cc(C)c2nc(C)cn2n1]

In some embodiments, A is

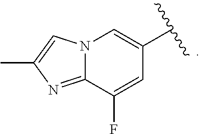 [SMILES: *c1cc(F)c2nc(C)cn2c1]

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

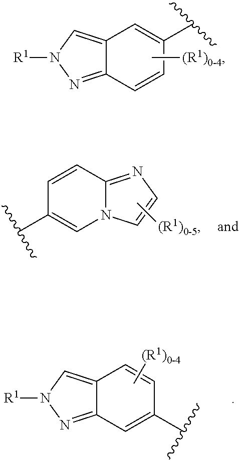

In some embodiments, B is selected from

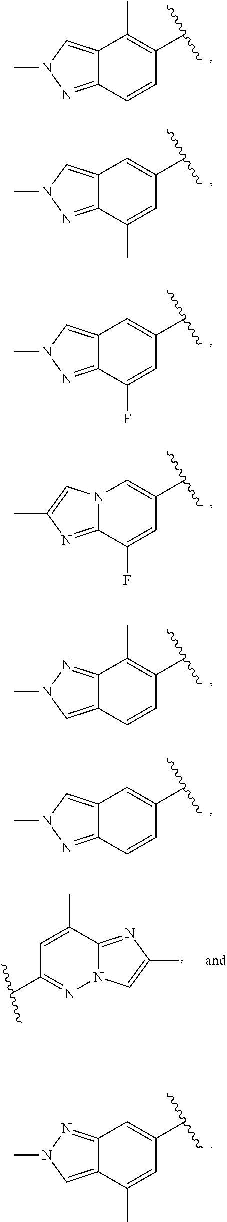

In some embodiments, B is heterocyclyl optionally substituted with one or more R¹. In some embodiments, B is monocyclic nitrogen-containing heterocyclyl. In some embodiments, B is selected from

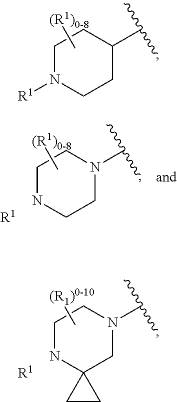

wherein R¹ is as defined herein.

In some embodiments, B is selected from

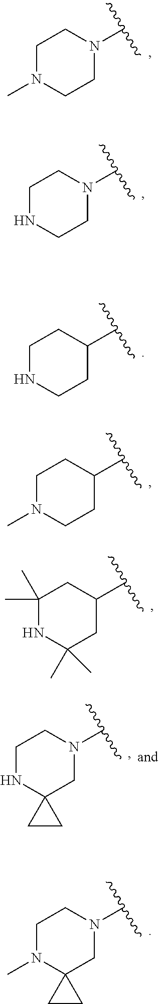

In some embodiments, B is

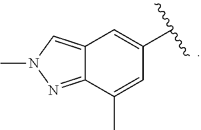 [SMILES: *c1cc(C)c2nn(C)cc2c1]

In some embodiments, B is

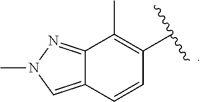 [SMILES: *c1ccc2cn(C)nc2c1C]

In some embodiments, B is

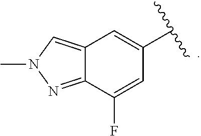 [SMILES: *c1cc(F)c2nn(C)cc2c1]

In some embodiments, B is

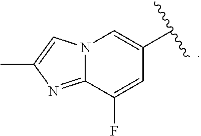 [SMILES: *c1cc(F)c2nc(C)cn2c1]

In some embodiments, B is

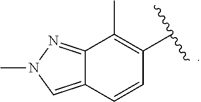 [SMILES: *c1ccc2cn(C)nc2c1C]

In some embodiments, B is

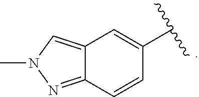 [SMILES: *c1ccc2nn(C)cc2c1]

In some embodiments, B is

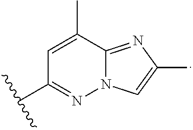

In some embodiments, B is

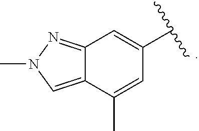 [SMILES: *c1cc(C)c2cn(C)nc2c1]

In some embodiments, B is

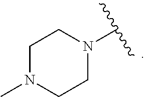 [SMILES: *N1CCN(C)CC1]

In some embodiments, B is

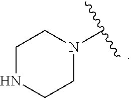 [SMILES: *N1CCNCC1]

In some embodiments, B is

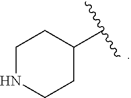 [SMILES: *C1CCNCC1]

In some embodiments, B is

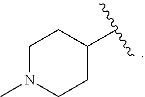 [SMILES: *C1CCN(C)CC1]

In some embodiments, B is

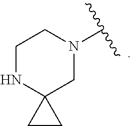 [SMILES: *N1CCNC2(CC2)C1]

In some embodiments, B is

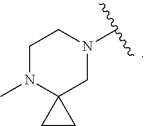 [SMILES: *N1CCN(C)C2(CC2)C1]

In some embodiments, B is

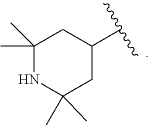 [SMILES: *C1CC(C)(C)NC(C)(C)C1]

In some embodiments, L¹ is absent or C₁-C₆-heteroalkylene. In some embodiments, L¹ is absent. In some embodiments, L¹ is C₁-C₆-heteroalkylene (e.g., —N(CH₃)—). In some embodiments, X is N. In some embodiments, X is C. In some embodiments, Y is N(R^{5a}) or C(R^{5b}). In some embodiments, Y is N(R^{5a}) (e.g., NH). In some embodiments, Y is C(R^{5b}) (e.g., CH). In some embodiments, X is C and Y is N(R^{5a}). In some embodiments, X is C and Y is NH. In some embodiments, X is N and Y is C(R^{5b}). In some embodiments, X is N and Y is CH.

In some embodiments, the compound of Formula (I) is a compound of Formula (I-c):

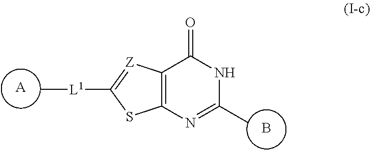

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; Z is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁶ is hydrogen, C₁-C₆-alkyl, C₁-C₆-heteroalkyl, C₁-C₆-haloalkyl, or halo; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸; each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is optionally substituted piperazinyl. In some embodiments, A is selected from

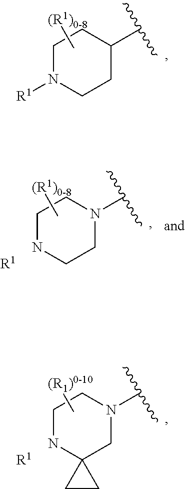

wherein R¹ is as defined herein.

In some embodiments, A is selected from

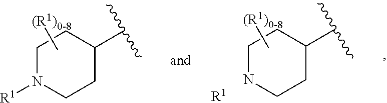

wherein R¹ is as defined herein. In some embodiments, A is selected from

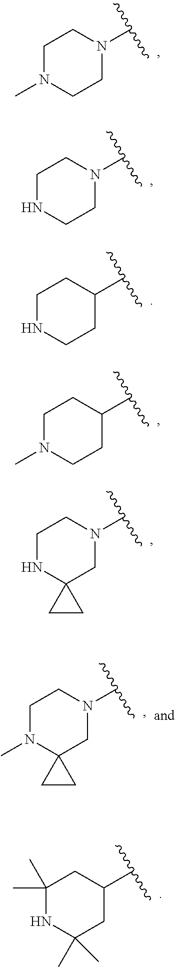

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

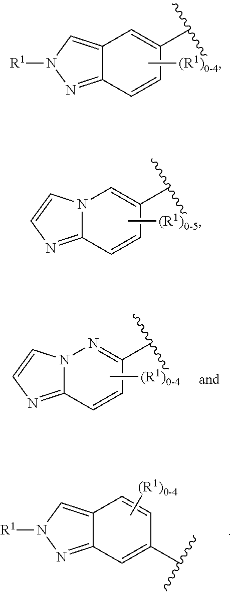

In some embodiments, A

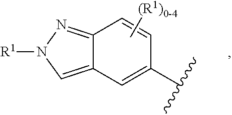

wherein R¹ is as defined herein.

In some embodiments, A is selected from

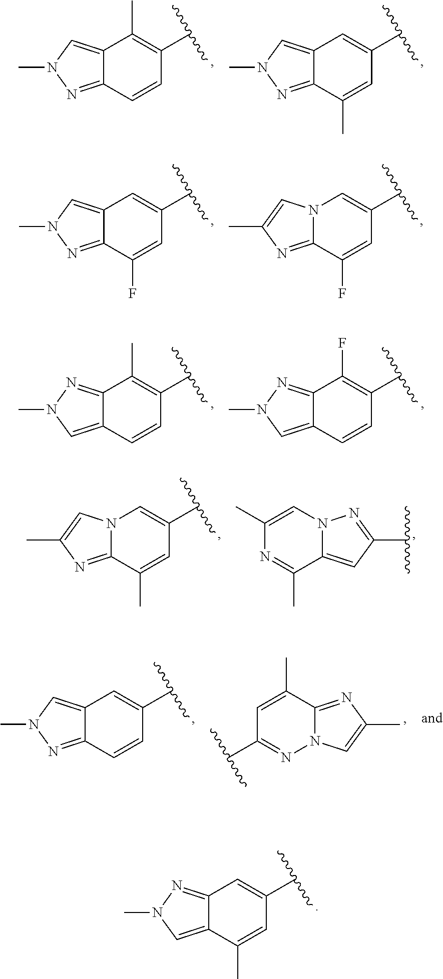

In some embodiments, A is

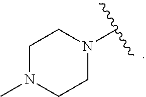 [SMILES: *N1CCN(C)CC1]

In some embodiments, A is

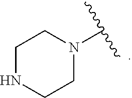 [SMILES: *N1CCNCC1]

In some embodiments, A is

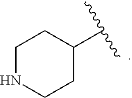 [SMILES: *C1CCNCC1]

In some embodiments, A is

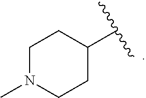 [SMILES: *C1CCN(C)CC1]

In some embodiments, A is

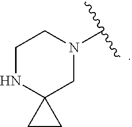 [SMILES: *N1CCNC2(CC2)C1]

In some embodiments, A is

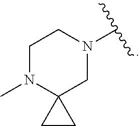 [SMILES: *N1CCN(C)C2(CC2)C1]

In some embodiments, A is

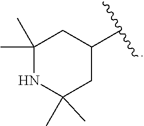 [SMILES: *C1CC(C)(C)NC(C)(C)C1]

In some embodiments A is

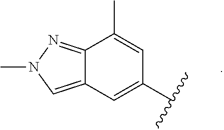 [SMILES: *c1cc(C)c2nn(C)cc2c1 ; "."]

In some embodiments, A is

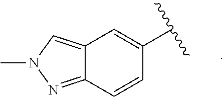 [SMILES: *c1ccc2nn(C)cc2c1 ; "."]

In some embodiments, A is

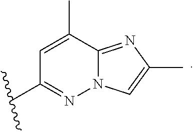 [SMILES: *c1cc(C)c2nc(C)cn2n1 ; "."]

In some embodiments, A is

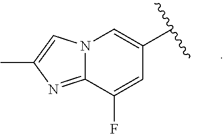 [SMILES: *c1cc(F)c2nc(C)cn2c1 ; "."]

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

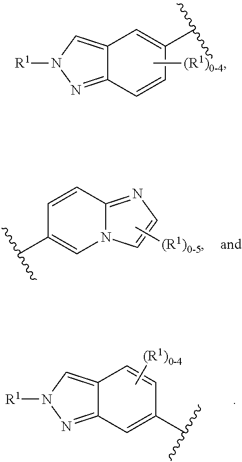

In some embodiments, B is selected from

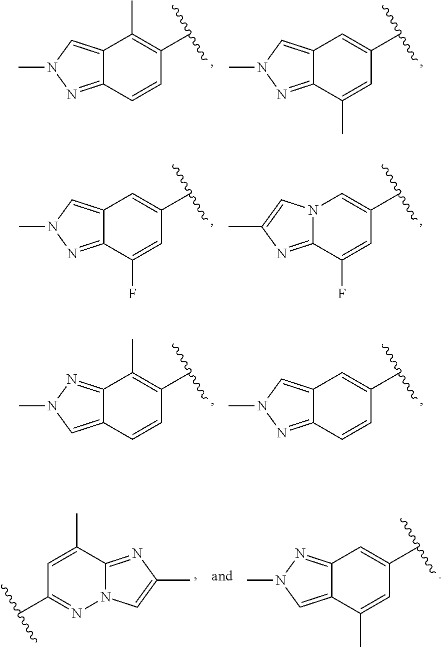

In some embodiments, B is heterocyclyl optionally substituted with one or more R¹. In some embodiments, B is monocyclic nitrogen-containing heterocyclyl. In some embodiments, B is selected from

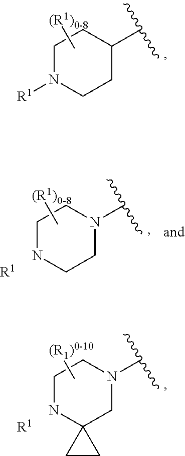

wherein R¹ is as defined herein.

In some embodiments, B is selected from

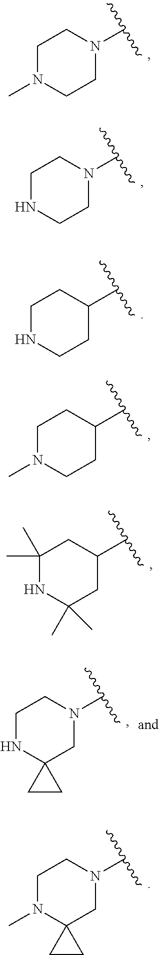

In some embodiments, B is

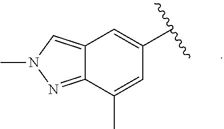 [SMILES: *c1cc(C)c2nn(C)cc2c1 ; "."]

In some embodiments, B is

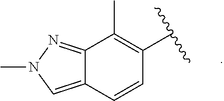 [SMILES: *c1ccc2cn(C)nc2c1C ; "."]

In some embodiments, B is

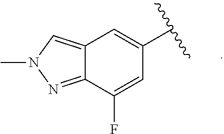 [SMILES: *c1cc(F)c2nn(C)cc2c1 ; "."]

In some embodiments, B is

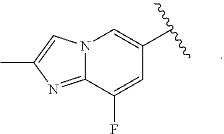 [SMILES: *c1cc(F)c2nc(C)cn2c1 ; "."]

In some embodiments, B is

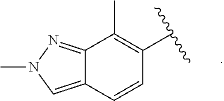 [SMILES: *c1ccc2cn(C)nc2c1C ; "."]

In some embodiments, B is

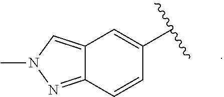 [SMILES: *c1ccc2nn(C)cc2c1 ; "."]

In some embodiments, B is

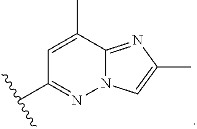 [SMILES: *c1cc(C)c2nc(C)cn2n1]

In some embodiments, B is

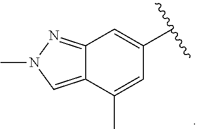 [SMILES: *c1cc(C)c2cn(C)nc2c1]

In some embodiments, B is

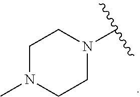 [SMILES: *N1CCN(C)CC1]

In some embodiments, B is

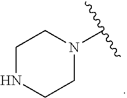 [SMILES: *N1CCNCC1]

In some embodiments, B is

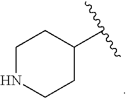 [SMILES: *C1CCNCC1]

In some embodiments, B is

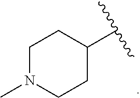 [SMILES: *C1CCN(C)CC1]

In some embodiments, B is

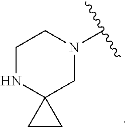 [SMILES: *N1CCNC2(CC2)C1]

In some embodiments, B is

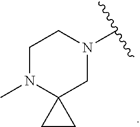 [SMILES: *N1CCN(C)C2(CC2)C1]

In some embodiments, B is

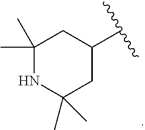 [SMILES: *C1CC(C)(C)NC(C)(C)C1]

In some embodiments, L¹ is absent or C₁-C₆-heteroalkylene. In some embodiments, L¹ is absent. In some embodiments, L¹ is C₁-C₆-heteroalkylene (e.g., —N(CH₃)—).

In some embodiments, Z is N. In some embodiments, Z is C(R⁶) (e.g., CH).

In some embodiments, the compound of Formula (I) is a compound of Formula (I-d):

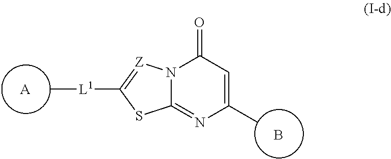

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; Z is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁶ is hydrogen, C₁-C₆-alkyl, C₁-C₆-heteroalkyl, C₁-C₆-haloalkyl, or halo; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸; each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is optionally substituted piperazinyl. In some embodiments, A is selected from

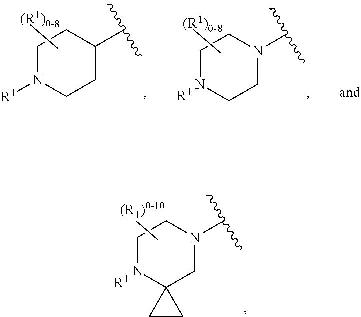

wherein R¹ is as defined herein.

In some embodiments, A is selected from

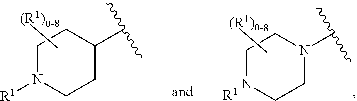

wherein R¹ is as defined herein. In some embodiments, A is selected from

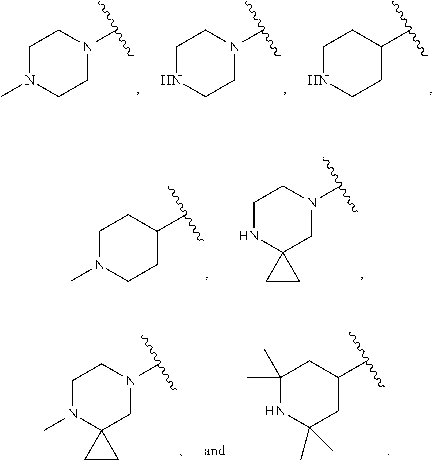

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

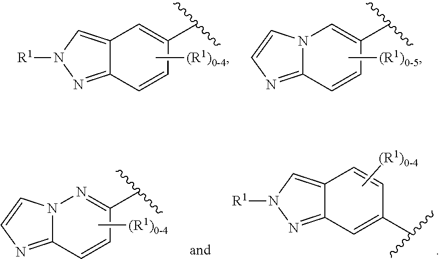

In some embodiments, A is

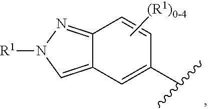

wherein R¹ is as defined herein.

In some embodiments, A is selected from

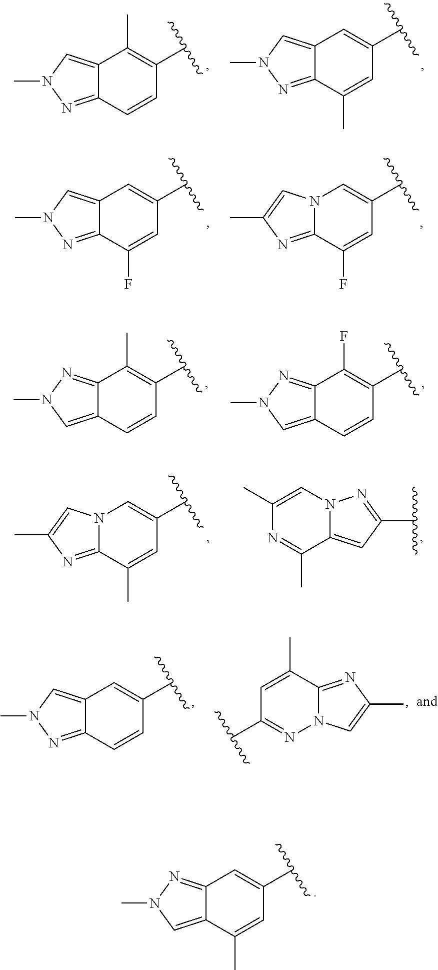

In some embodiments, A is

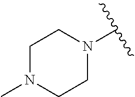 [SMILES: *N1CCN(C)CC1]

In some embodiments, A is

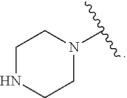 [SMILES: *N1CCNCC1]

In some embodiments, A is

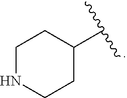 [SMILES: *C1CCNCC1]

In some embodiments, A is

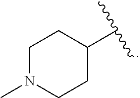 [SMILES: *C1CCN(C)CC1]

In some embodiments, A is

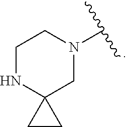 [SMILES: *N1CCNC2(CC2)C1]

In some embodiments, A is

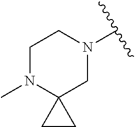 [SMILES: *N1CCN(C)C2(CC2)C1]

In some embodiments, A is

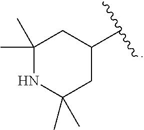 [SMILES: *C1CC(C)(C)NC(C)(C)C1]

In some embodiments, A is

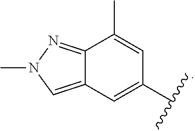 [SMILES: *c1cc(C)c2nn(C)cc2c1]

In some embodiments, A is

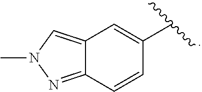 [SMILES: *c1ccc2nn(C)cc2c1]

In some embodiments, A is

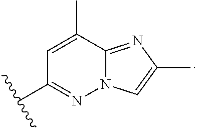 [SMILES: *c1cc(C)c2nc(C)cn2n1]

In some embodiments, A is

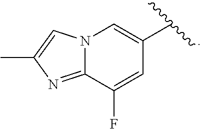 [SMILES: *c1cc(F)c2nc(C)cn2c1]

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

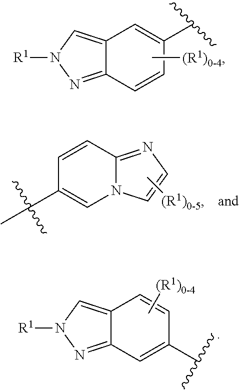

In some embodiments, B is selected from

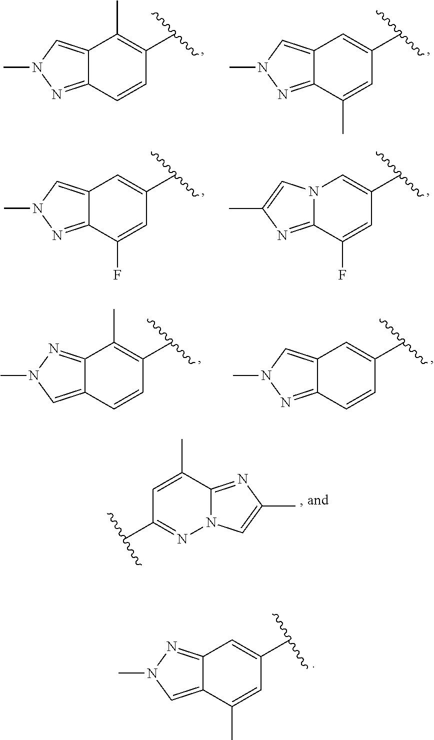

In some embodiments, B is heterocyclyl optionally substituted with one or more R¹. In some embodiments, B is monocyclic nitrogen-containing heterocyclyl. In some embodiments, B is selected from

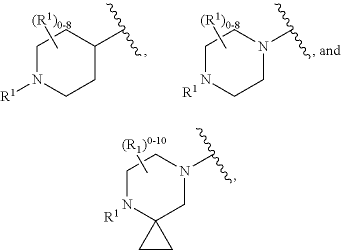

and wherein R¹ is as defined herein.

In some embodiments, B is selected from

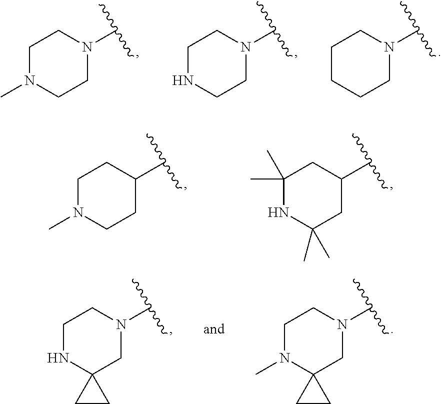

In some embodiments, B is

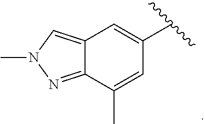 [SMILES: *c1cc(C)c2nn(C)cc2c1]

In some embodiments, B is

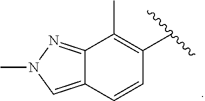 [SMILES: *c1ccc2cn(C)nc2c1C]

In some embodiments, B is

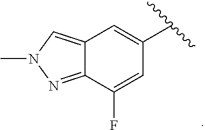 [SMILES: *c1cc(F)c2nn(C)cc2c1]

In some embodiments, B is

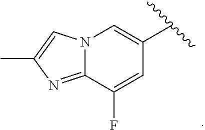 [SMILES: *c1cc(F)c2nc(C)cn2c1]

In some embodiments, B is

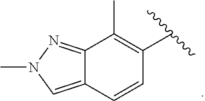 [SMILES: *c1ccc2cn(C)nc2c1C]

In some embodiments, B is

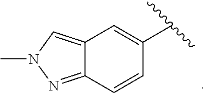 [SMILES: *c1ccc2nn(C)cc2c1]

In some embodiments, B is

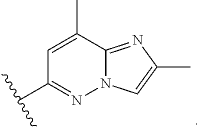 [SMILES: *c1cc(C)c2nc(C)cn2n1]

In some embodiments, B is

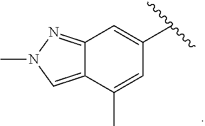 [SMILES: *c1cc(C)c2cn(C)nc2c1]

In some embodiments, B is

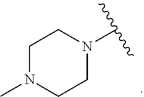 [SMILES: *N1CCN(C)CC1]

In some embodiments, B is

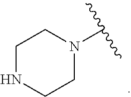 [SMILES: *N1CCNCC1]

In some embodiments, B is

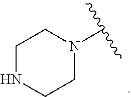 [SMILES: *N1CCNCC1]

In some embodiments, B is

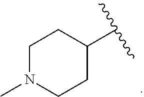 [SMILES: *C1CCN(C)CC1]

In some embodiments, B is

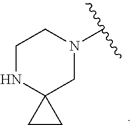 [SMILES: *N1CCNC2(CC2)C1]

In some embodiments, B is

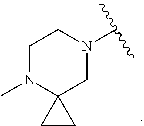 [SMILES: *N1CCN(C)C2(CC2)C1]

In some embodiments, B is

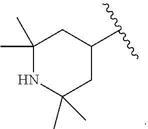 [SMILES: *C1CC(C)(C)NC(C)(C)C1]

In some embodiments, L¹ is absent or C₁-C₆-heteroalkylene. In some embodiments, L¹ is absent. In some embodiments, L¹ is C₁-C₆-heteroalkylene (e.g., —N(CH₃)—).

In some embodiments, Z is N. In some embodiments, Z is C(R⁶) (e.g., CH).

In some embodiments, the compound of Formula (I) is a compound of Formula (I-e):

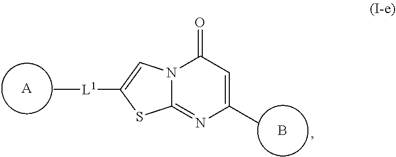

or a pharmaceutically acceptable salt, solvate, hydrate, tautomer, or stereoisomer thereof, wherein A, B, L¹, and subvariables thereof are as defined herein.

In some embodiments, the compound of Formula (I) is a compound of Formula (I-f):

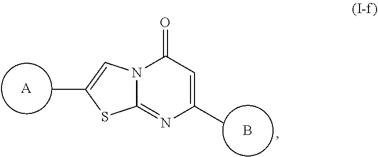

or a pharmaceutically acceptable salt, solvate, hydrate, tautomer, or stereoisomer thereof, wherein A is a nitrogen-containing 5-6 membered heterocyclyl or 5-6 membered cycloalkyl, each of which is optionally substituted with one or more R¹; and B is a bicyclic nitrogen-containing heteroaryl, optionally substituted with one or more R¹.

In some embodiments, the compound of Formula (I) is a compound of Formula (I-g):

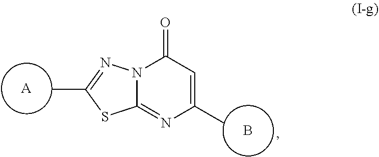

or a pharmaceutically acceptable salt, solvate, hydrate, tautomer, or stereoisomer thereof, wherein A is a nitrogen-containing 5-6 membered heterocyclyl or 5-6 membered cycloalkyl, each of which is optionally substituted with one or more R¹; and B is a bicyclic nitrogen-containing heteroaryl, optionally substituted with one or more R¹.

In some embodiments, the compound of Formula (I) is a compound of Formula (I-h):

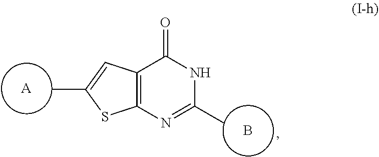

or a pharmaceutically acceptable salt, solvate, hydrate, tautomer, or stereoisomer thereof, wherein A is a nitrogen-containing 5-6 membered heterocyclyl or 5-6 membered cycloalkyl, each of which is optionally substituted with one or more R¹; and B is a bicyclic nitrogen-containing heteroaryl, optionally substituted with one or more R¹.

In some embodiments, the compound of Formula (I) is selected from a compound in Table 1, or a pharmaceutically acceptable salt, solvate, hydrate, tautomer, or stereoisomer thereof.

TABLE 1
Exemplary compounds of Formula (I).

| Cmpd | |
|---|---|
| No. | Structure |
| 100 | 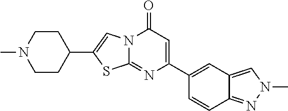 |
| 101 | 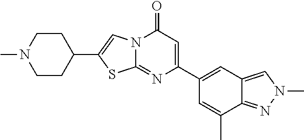 |
| 102 | 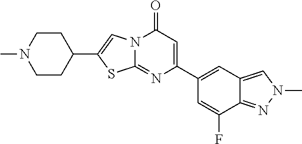 |
| 103 | 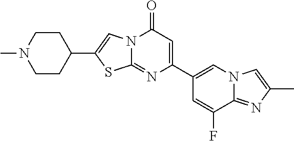 |
| 104 | 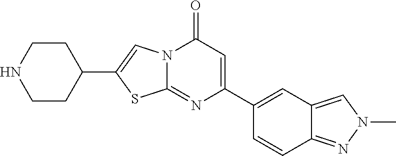 [SMILES: Cn1cc2cc(-c3cc(=O)n4cc(C5CCNCC5)sc4n3)ccc2n1] |
| 105 | 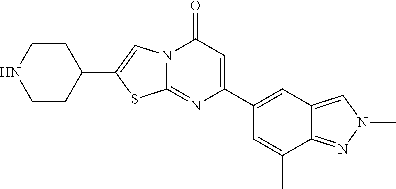 [SMILES: Cc1cc(-c2cc(=O)n3cc(C4CCNCC4)sc3n2)cc2cn(C)nc12] |
| 106 | 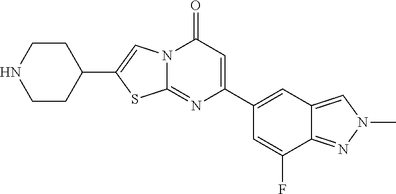 [SMILES: Cn1cc2cc(-c3cc(=O)n4cc(C5CCNCC5)sc4n3)cc(F)c2n1] |
| 107 | 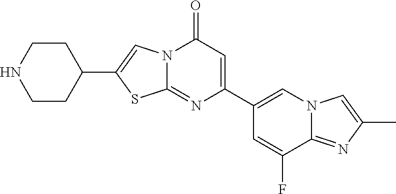 [SMILES: Cc1cn2cc(-c3cc(=O)n4cc(C5CCNCC5)sc4n3)cc(F)c2n1] |
| 108 | 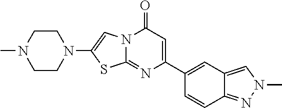 |
| 109 | 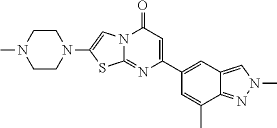 |
| 110 | 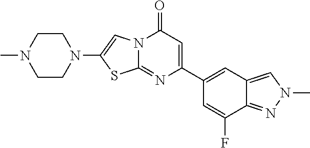 |
| 111 | 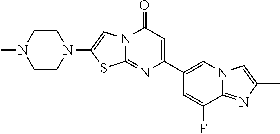 |
| 112 | 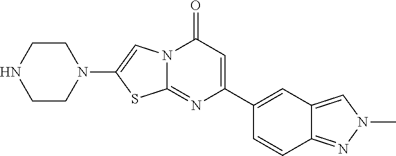 [SMILES: Cn1cc2cc(-c3cc(=O)n4cc(N5CCNCC5)sc4n3)ccc2n1] |
| 113 | 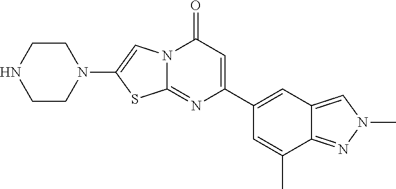 [SMILES: Cc1cc(-c2cc(=O)n3cc(N4CCNCC4)sc3n2)cc2cn(C)nc12] |
| 114 | 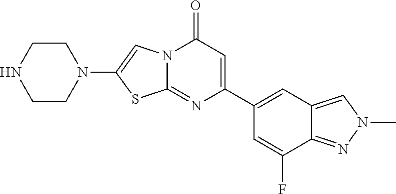 [SMILES: Cn1cc2cc(-c3cc(=O)n4cc(N5CCNCC5)sc4n3)cc(F)c2n1] |
| 115 | 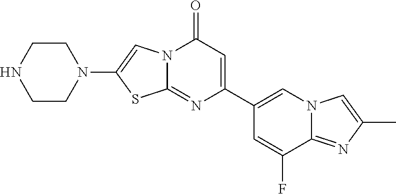 [SMILES: Cc1cn2cc(-c3cc(=O)n4cc(N5CCNCC5)sc4n3)cc(F)c2n1] |
| 116 | 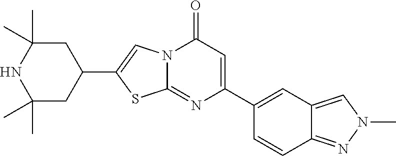 |
| 117 | 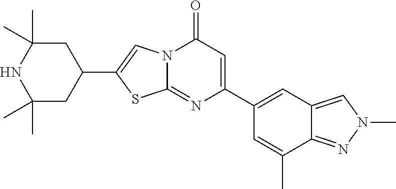 |
| 118 | 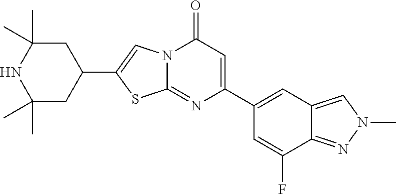 |
| 119 | 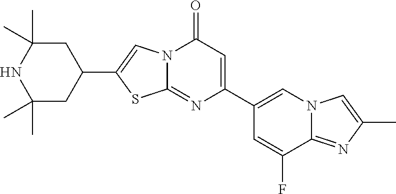 |
| 120 | 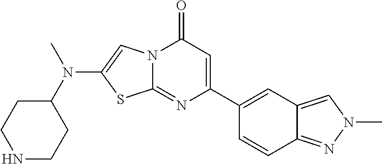 [SMILES: CN(c1cn2c(=O)cc(-c3ccc4nn(C)cc4c3)nc2s1)C1CCNCC1] |
| 121 | 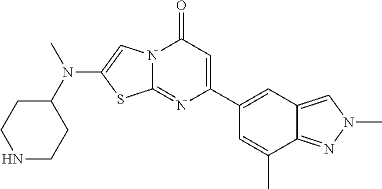 [SMILES: Cc1cc(-c2cc(=O)n3cc(N(C)C4CCNCC4)sc3n2)cc2cn(C)nc12] |
| 122 | 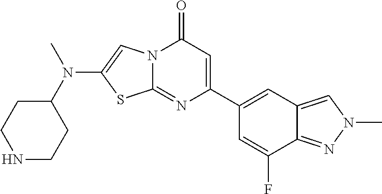 [SMILES: CN(c1cn2c(=O)cc(-c3cc(F)c4nn(C)cc4c3)nc2s1)C1CCNCC1] |
| 123 | 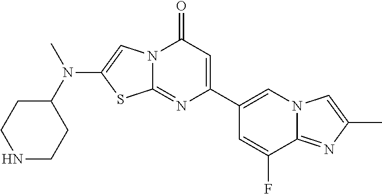 [SMILES: Cc1cn2cc(-c3cc(=O)n4cc(N(C)C5CCNCC5)sc4n3)cc(F)c2n1] |
| 124 | 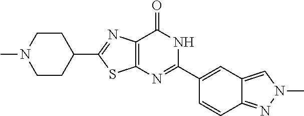 |
| 125 | 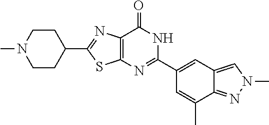 |
| 126 | 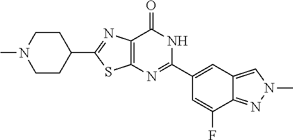 |
| 127 | 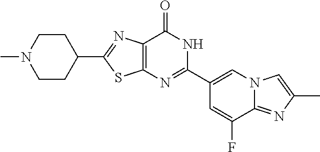 |
| 128 | 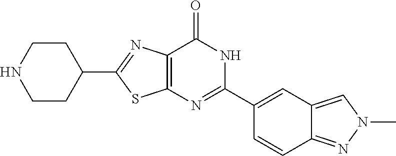 [SMILES: Cn1cc2cc(-c3nc4sc(C5CCNCC5)nc4c(=O)[nH]3)ccc2n1] |
| 129 | 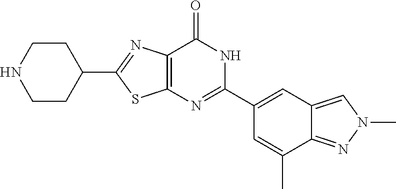 [SMILES: Cc1cc(-c2nc3sc(C4CCNCC4)nc3c(=O)[nH]2)cc2cn(C)nc12] |
| 130 | 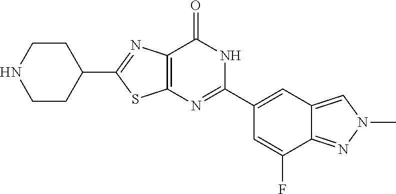 [SMILES: Cn1cc2cc(-c3nc4sc(C5CCNCC5)nc4c(=O)[nH]3)cc(F)c2n1] |
| 131 | 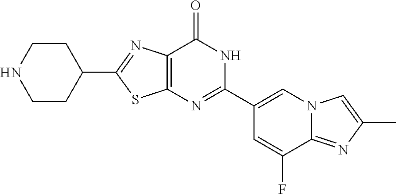 [SMILES: Cc1cn2cc(-c3nc4sc(C5CCNCC5)nc4c(=O)[nH]3)cc(F)c2n1] |
| 132 | 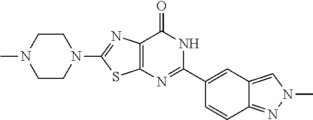 |
| 133 | 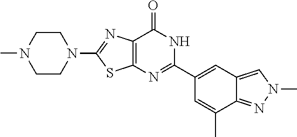 |
| 134 | 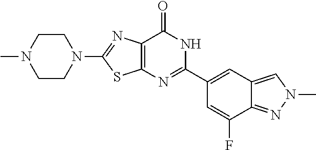 |
| 135 | 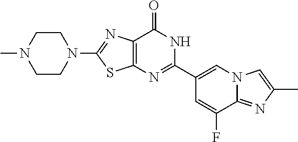 |
| 136 | 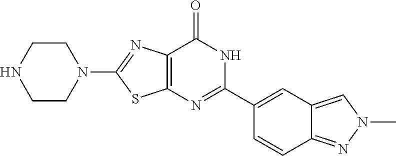 [SMILES: Cn1cc2cc(-c3nc4sc(N5CCNCC5)nc4c(=O)[nH]3)ccc2n1] |
| 137 | 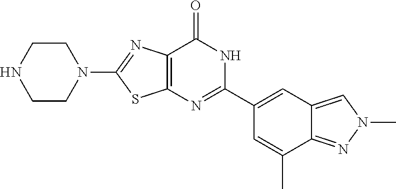 [SMILES: Cc1cc(-c2nc3sc(N4CCNCC4)nc3c(=O)[nH]2)cc2cn(C)nc12] |
| 138 | 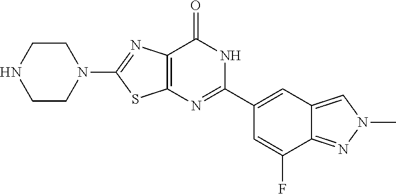 [SMILES: Cn1cc2cc(-c3nc4sc(N5CCNCC5)nc4c(=O)[nH]3)cc(F)c2n1] |
| 139 | 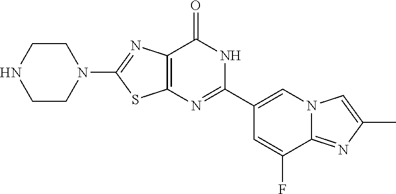 [SMILES: Cc1cn2cc(-c3nc4sc(N5CCNCC5)nc4c(=O)[nH]3)cc(F)c2n1] |
| 140 | 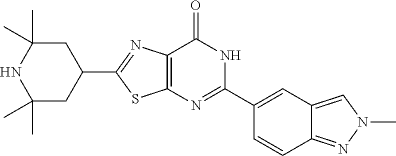 |
| 141 | 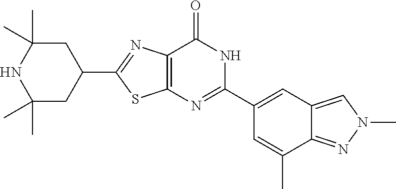 |
| 142 | 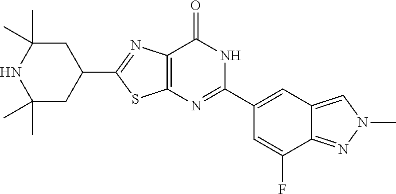 |
| 143 | 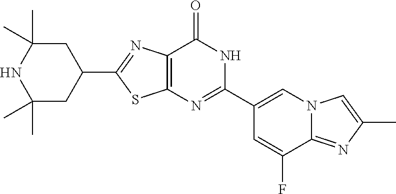 |
| 144 | 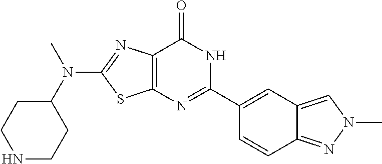 [SMILES: CN(c1nc2c(=O)[nH]c(-c3ccc4nn(C)cc4c3)nc2s1)C1CCNCC1] |
| 145 | 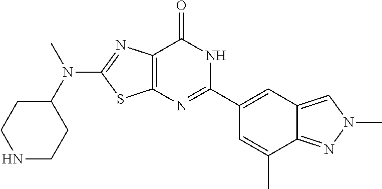 [SMILES: Cc1cc(-c2nc3sc(N(C)C4CCNCC4)nc3c(=O)[nH]2)cc2cn(C)nc12] |
| 146 | 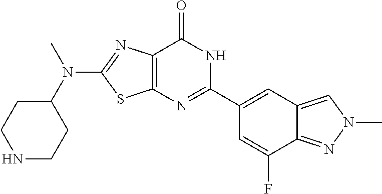 [SMILES: CN(c1nc2c(=O)[nH]c(-c3cc(F)c4nn(C)cc4c3)nc2s1)C1CCNCC1] |
| 147 | 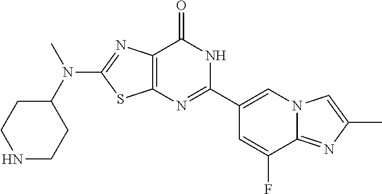 [SMILES: Cc1cn2cc(-c3nc4sc(N(C)C5CCNCC5)nc4c(=O)[nH]3)cc(F)c2n1] |
| 148 | 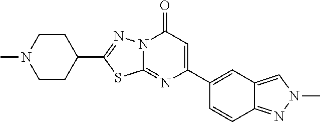 |
| 149 | 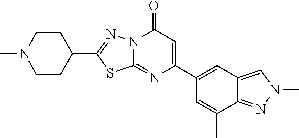 |
| 150 | 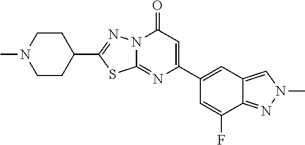 |
| 151 | 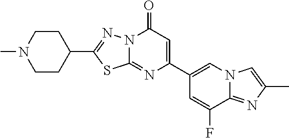 |
| 152 | 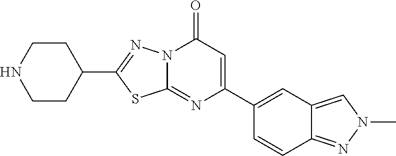 [SMILES: Cn1cc2cc(-c3cc(=O)n4nc(C5CCNCC5)sc4n3)ccc2n1] |
| 153 | 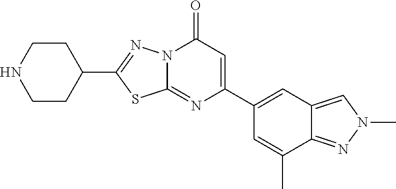 [SMILES: Cc1cc(-c2cc(=O)n3nc(C4CCNCC4)sc3n2)cc2cn(C)nc12] |
| 154 | 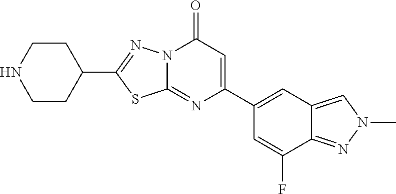 [SMILES: Cn1cc2cc(-c3cc(=O)n4nc(C5CCNCC5)sc4n3)cc(F)c2n1] |
| 155 | 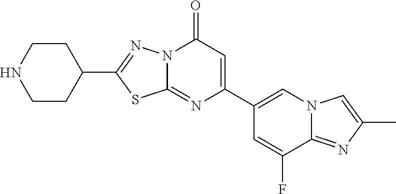 [SMILES: Cc1cn2cc(-c3cc(=O)n4nc(C5CCNCC5)sc4n3)cc(F)c2n1] |
| 156 | 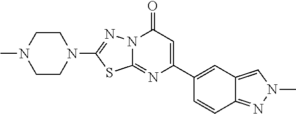 |
| 157 | 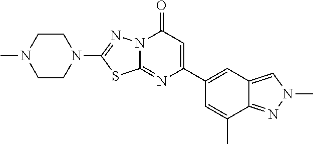 |
| 158 | 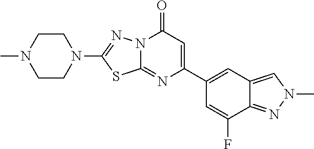 |
| 159 | 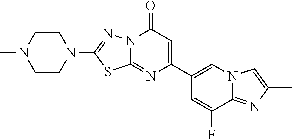 |
| 160 | 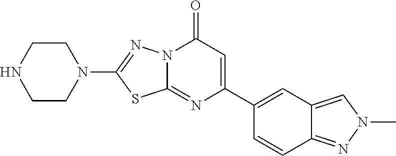 [SMILES: Cn1cc2cc(-c3cc(=O)n4nc(N5CCNCC5)sc4n3)ccc2n1] |
| 161 | 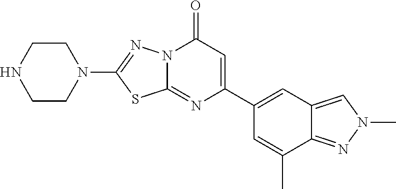 [SMILES: Cc1cc(-c2cc(=O)n3nc(N4CCNCC4)sc3n2)cc2cn(C)nc12] |
| 162 | 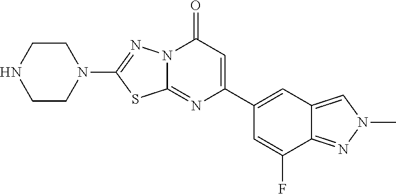 [SMILES: Cn1cc2cc(-c3cc(=O)n4nc(N5CCNCC5)sc4n3)cc(F)c2n1] |
| 163 | 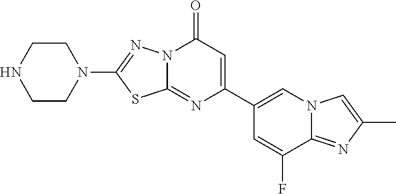 [SMILES: Cc1cn2cc(-c3cc(=O)n4nc(N5CCNCC5)sc4n3)cc(F)c2n1] |
| 164 | 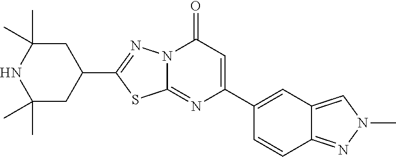 |
| 165 | 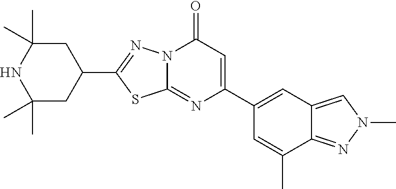 |
| 166 | 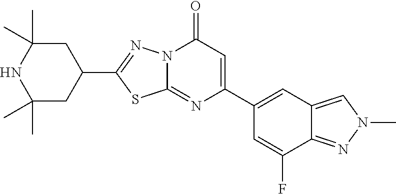 |
| 167 | 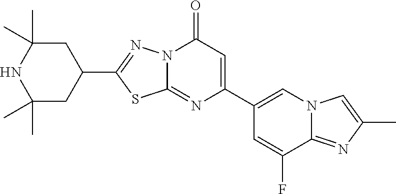 |
| 168 | 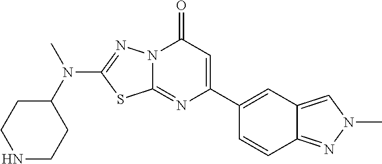 [SMILES: CN(c1nn2c(=O)cc(-c3ccc4nn(C)cc4c3)nc2s1)C1CCNCC1] |
| 169 | 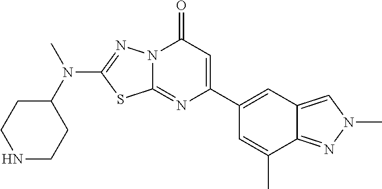 [SMILES: Cc1cc(-c2cc(=O)n3nc(N(C)C4CCNCC4)sc3n2)cc2cn(C)nc12] |
| 170 | 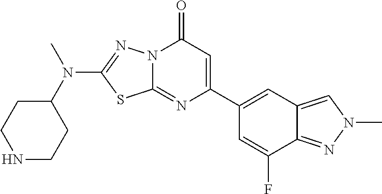 [SMILES: CN(c1nn2c(=O)cc(-c3cc(F)c4nn(C)cc4c3)nc2s1)C1CCNCC1] |
| 171 | 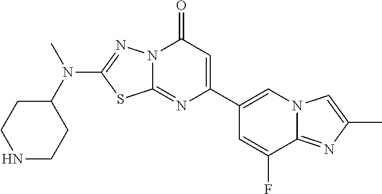 [SMILES: Cc1cn2cc(-c3cc(=O)n4nc(N(C)C5CCNCC5)sc4n3)cc(F)c2n1] |
| 183 | 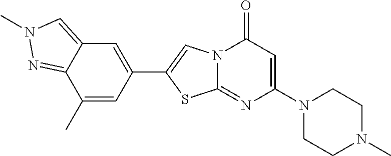 |
| 184 | 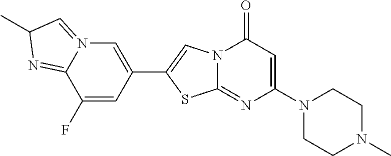 |
| 192 | 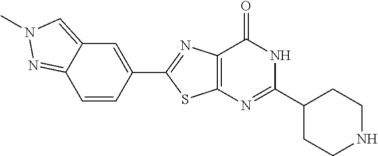 [SMILES: Cn1cc2cc(-c3nc4c(=O)[nH]c(C5CCNCC5)nc4s3)ccc2n1] |
| 193 | 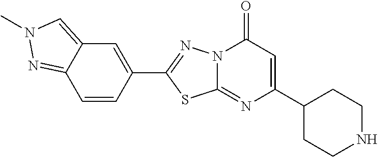 [SMILES: Cn1cc2cc(-c3nn4c(=O)cc(C5CCNCC5)nc4s3)ccc2n1] |
| 194 | 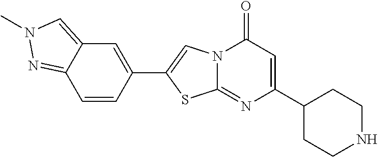 [SMILES: Cn1cc2cc(-c3cn4c(=O)cc(C5CCNCC5)nc4s3)ccc2n1] |
| 205 | 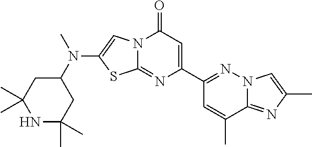 |
| 206 | 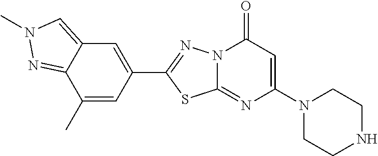 [SMILES: Cc1cc(-c2nn3c(=O)cc(N4CCNCC4)nc3s2)cc2cn(C)nc12] |
| 207 | 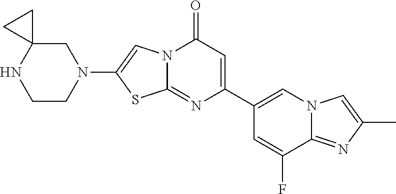 [SMILES: Cc1cn2cc(-c3cc(=O)n4cc(N5CCNC6(CC6)C5)sc4n3)cc(F)c2n1] |
| 208 | 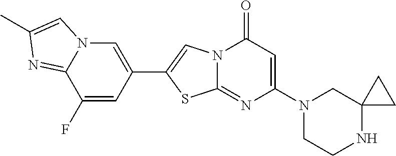 [SMILES: Cc1cn2cc(-c3cn4c(=O)cc(N5CCNC6(CC6)C5)nc4s3)cc(F)c2n1] |
| 209 | 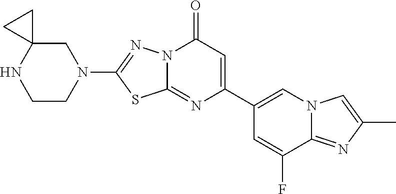 [SMILES: Cc1cn2cc(-c3cc(=O)n4nc(N5CCNC6(CC6)C5)sc4n3)cc(F)c2n1] |
| 210 | 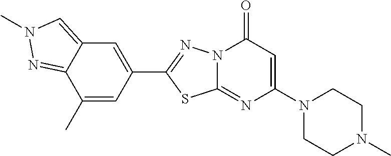 |
| 211 | 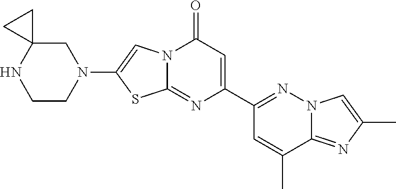 [SMILES: Cc1cn2nc(-c3cc(=O)n4cc(N5CCNC6(CC6)C5)sc4n3)cc(C)c2n1] |
| 212 | 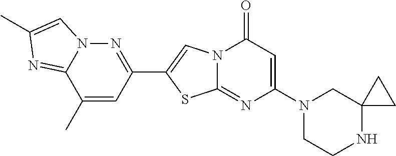 [SMILES: Cc1cn2nc(-c3cn4c(=O)cc(N5CCNC6(CC6)C5)nc4s3)cc(C)c2n1] |
| 213 | 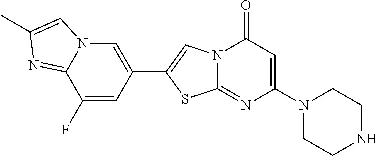 [SMILES: Cc1cn2cc(-c3cn4c(=O)cc(N5CCNCC5)nc4s3)cc(F)c2n1] |
| 214 | 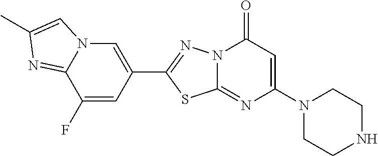 [SMILES: Cc1cn2cc(-c3nn4c(=O)cc(N5CCNCC5)nc4s3)cc(F)c2n1] |
| 215 | 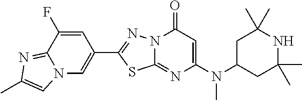 |
| 216 | 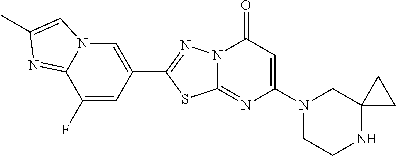 [SMILES: Cc1cn2cc(-c3nn4c(=O)cc(N5CCNC6(CC6)C5)nc4s3)cc(F)c2n1] |
| 217 | 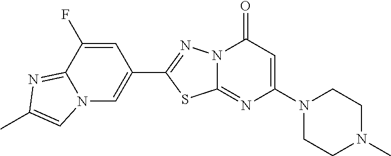 |
| 218 | 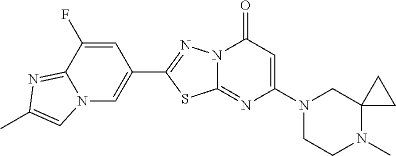 [SMILES: Cc1cn2cc(-c3nn4c(=O)cc(N5CCN(C)C6(CC6)C5)nc4s3)cc(F)c2n1] |
| 250 | 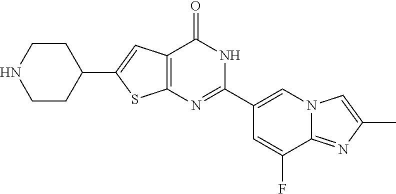 [SMILES: Cc1cn2cc(-c3nc4sc(C5CCNCC5)cc4c(=O)[nH]3)cc(F)c2n1] |
| 256 | 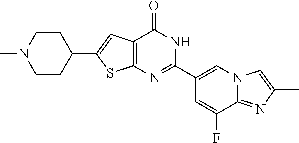 |
| 267 | 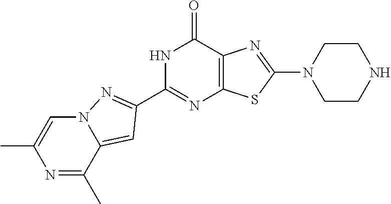 [SMILES: Cc1cn2nc(-c3nc4sc(N5CCNCC5)nc4c(=O)[nH]3)cc2c(C)n1] |
| 268 | 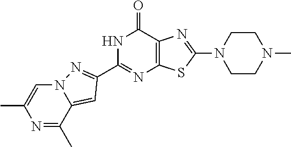 |
| 282 | 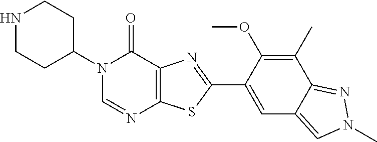 [SMILES: COc1c(-c2nc3c(=O)n(C4CCNCC4)cnc3s2)cc2cn(C)nc2c1C] |
| 283 | 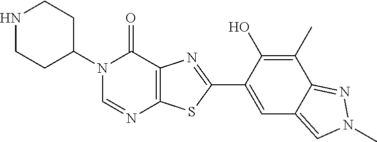 [SMILES: Cc1c(O)c(-c2nc3c(=O)n(C4CCNCC4)cnc3s2)cc2cn(C)nc12] |
| 284 | 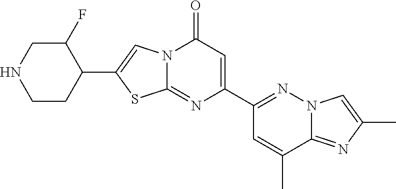 [SMILES: Cc1cn2nc(-c3cc(=O)n4cc(C5CCNCC5F)sc4n3)cc(C)c2n1] |
| 285 | 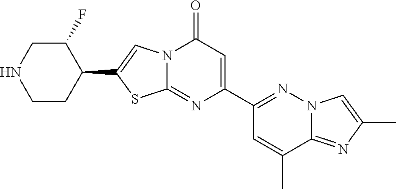 [SMILES: Cc1cn2nc(-c3cc(=O)n4cc([C@H]5CCNC[C@@H]5F)sc4n3)cc(C)c2n1] |
| 286 | 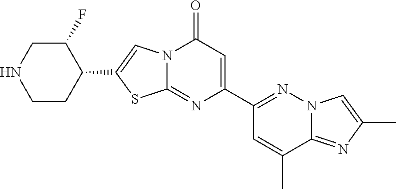 [SMILES: Cc1cn2nc(-c3cc(=O)n4cc([C@@H]5CCNC[C@@H]5F)sc4n3)cc(C)c2n1] |
| 287 | 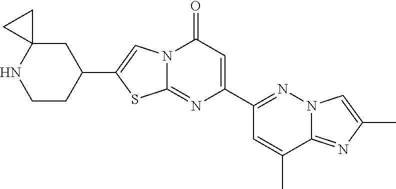 [SMILES: Cc1cn2nc(-c3cc(=O)n4cc(C5CCNC6(CC6)C5)sc4n3)cc(C)c2n1] |
| 288 | 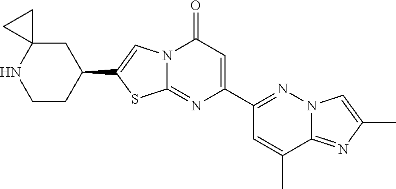 [SMILES: Cc1cn2nc(-c3cc(=O)n4cc([C@H]5CCNC6(CC6)C5)sc4n3)cc(C)c2n1] |
| 289 | 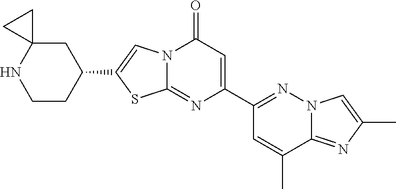 [SMILES: Cc1cn2nc(-c3cc(=O)n4cc([C@@H]5CCNC6(CC6)C5)sc4n3)cc(C)c2n1] |
| 290 | 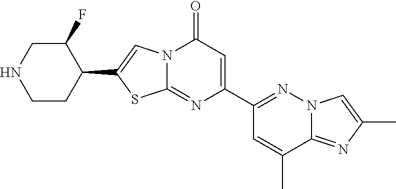 [SMILES: Cc1cn2nc(-c3cc(=O)n4cc([C@H]5CCNC[C@H]5F)sc4n3)cc(C)c2n1] |
| 291 | 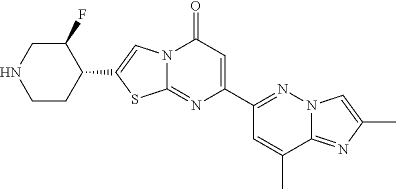 [SMILES: Cc1cn2nc(-c3cc(=O)n4cc([C@@H]5CCNC[C@H]5F)sc4n3)cc(C)c2n1] |
| 292 | 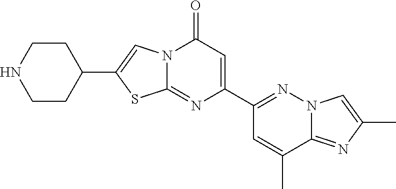 [SMILES: Cc1cn2nc(-c3cc(=O)n4cc(C5CCNCC5)sc4n3)cc(C)c2n1] |
| 293 | 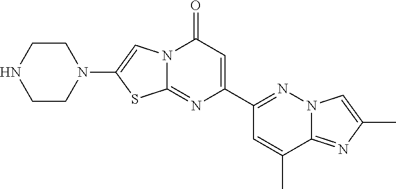 [SMILES: Cc1cn2nc(-c3cc(=O)n4cc(N5CCNCC5)sc4n3)cc(C)c2n1] |
| 294 | 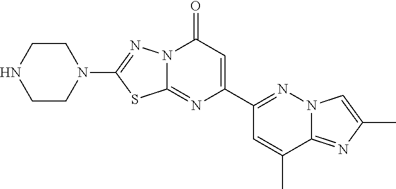 [SMILES: Cc1cn2nc(-c3cc(=O)n4nc(N5CCNCC5)sc4n3)cc(C)c2n1] |
| 295 | 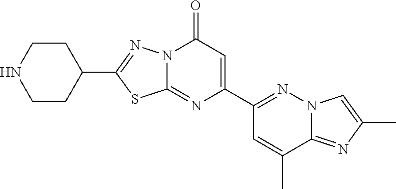 [SMILES: Cc1cn2nc(-c3cc(=O)n4nc(C5CCNCC5)sc4n3)cc(C)c2n1] |
| 296 | 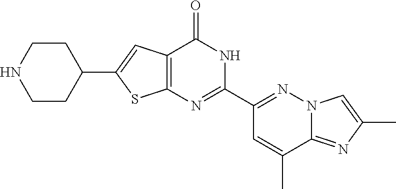 [SMILES: Cc1cn2nc(-c3nc4sc(C5CCNCC5)cc4c(=O)[nH]3)cc(C)c2n1] |
| 297 | 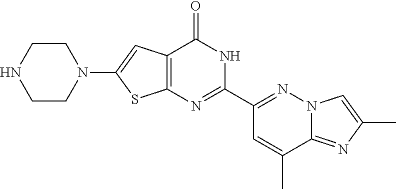 [SMILES: Cc1cn2nc(-c3nc4sc(N5CCNCC5)cc4c(=O)[nH]3)cc(C)c2n1] |
| 298 | 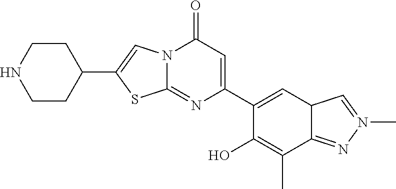 |
| 299 | 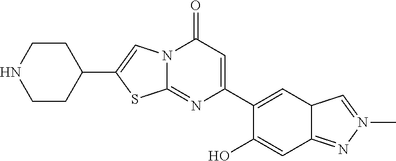 |
| 300 | 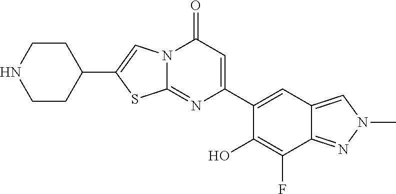 [SMILES: Cn1cc2cc(-c3cc(=O)n4cc(C5CCNCC5)sc4n3)c(O)c(F)c2n1] |
| 301 | 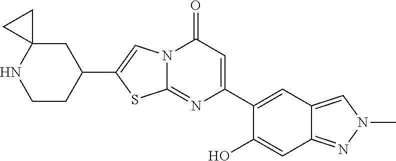 [SMILES: Cn1cc2cc(-c3cc(=O)n4cc(C5CCNC6(CC6)C5)sc4n3)c(O)cc2n1] |
| 302 | 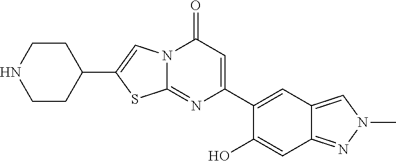 [SMILES: Cn1cc2cc(-c3cc(=O)n4cc(C5CCNCC5)sc4n3)c(O)cc2n1] |
| 303 | 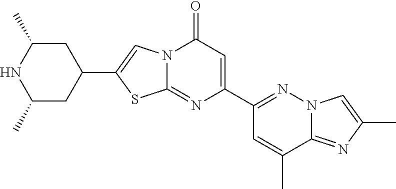 [SMILES: Cc1cn2nc(-c3cc(=O)n4cc(C5C[C@@H](C)N[C@@H](C)C5)sc4n3)cc(C)c2n1] |
| 304 | 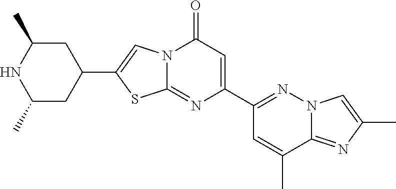 [SMILES: Cc1cn2nc(-c3cc(=O)n4cc(C5C[C@H](C)N[C@@H](C)C5)sc4n3)cc(C)c2n1] |
| 305 | 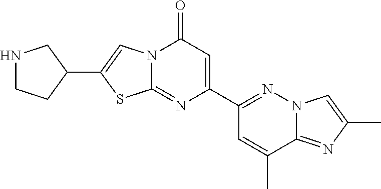 [SMILES: Cc1cn2nc(-c3cc(=O)n4cc(C5CCNC5)sc4n3)cc(C)c2n1] |
| 306 | 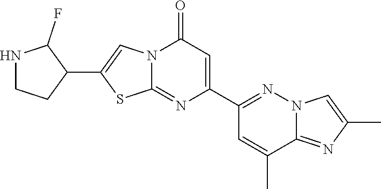 [SMILES: Cc1cn2nc(-c3cc(=O)n4cc(C5CCNC5F)sc4n3)cc(C)c2n1] |
| 307 | 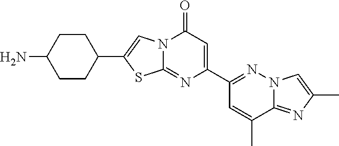 [SMILES: Cc1cn2nc(-c3cc(=O)n4cc(C5CCC(N)CC5)sc4n3)cc(C)c2n1] |
| 308 | 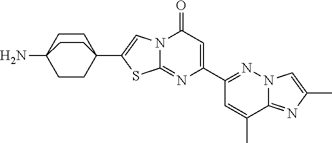 [SMILES: Cc1cn2nc(-c3cc(=O)n4cc(C56CCC(N)(CC5)CC6)sc4n3)cc(C)c2n1] |
| 309 | 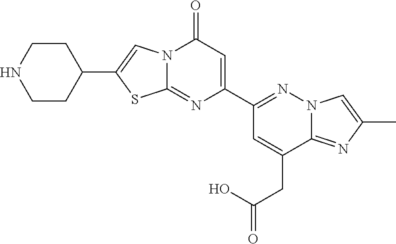 [SMILES: Cc1cn2nc(-c3cc(=O)n4cc(C5CCNCC5)sc4n3)cc(CC(=O)O)c2n1] |
| 310 | 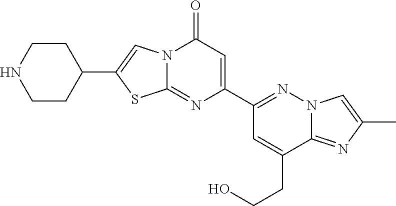 [SMILES: Cc1cn2nc(-c3cc(=O)n4cc(C5CCNCC5)sc4n3)cc(CCO)c2n1] |
| 311 | 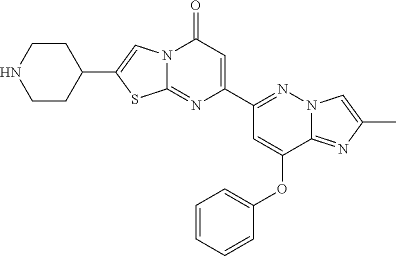 [SMILES: Cc1cn2nc(-c3cc(=O)n4cc(C5CCNCC5)sc4n3)cc(Oc3ccccc3)c2n1] |
| 312 | 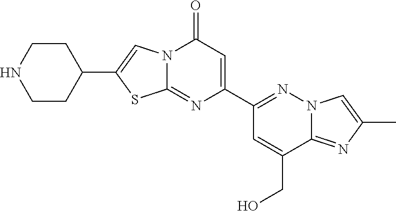 [SMILES: Cc1cn2nc(-c3cc(=O)n4cc(C5CCNCC5)sc4n3)cc(CO)c2n1] |
| 313 | 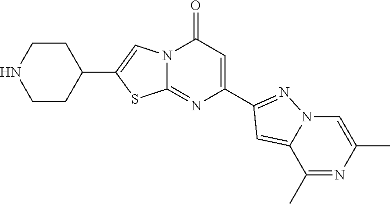 [SMILES: Cc1cn2nc(-c3cc(=O)n4cc(C5CCNCC5)sc4n3)cc2c(C)n1] |
| 314 | 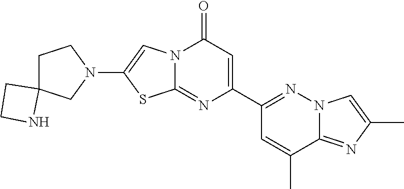 [SMILES: Cc1cn2nc(-c3cc(=O)n4cc(N5CCC6(CCN6)C5)sc4n3)cc(C)c2n1] |
| 315 | 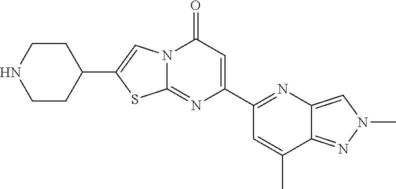 [SMILES: Cc1cc(-c2cc(=O)n3cc(C4CCNCC4)sc3n2)nc2cn(C)nc12] |
| 316 | 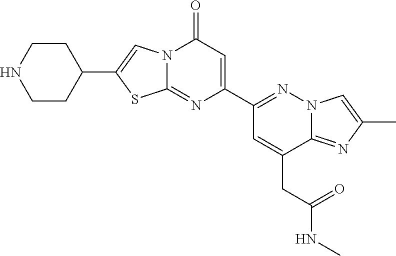 [SMILES: CNC(=O)Cc1cc(-c2cc(=O)n3cc(C4CCNCC4)sc3n2)nn2cc(C)nc12] |
| 317 | 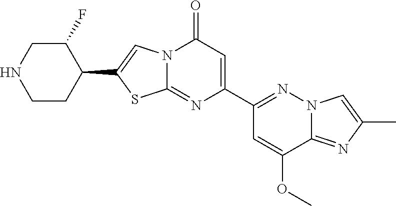 [SMILES: COc1cc(-c2cc(=O)n3cc([C@H]4CCNC[C@@H]4F)sc3n2)nn2cc(C)nc12] |
| 318 | 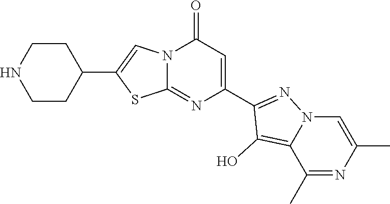 [SMILES: Cc1cn2nc(-c3cc(=O)n4cc(C5CCNCC5)sc4n3)c(O)c2c(C)n1] |
| 319 | 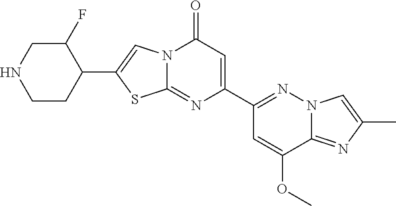 [SMILES: COc1cc(-c2cc(=O)n3cc(C4CCNCC4F)sc3n2)nn2cc(C)nc12] |
| 320 | 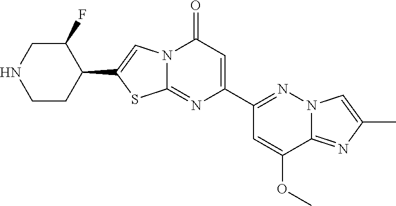 [SMILES: COc1cc(-c2cc(=O)n3cc([C@H]4CCNC[C@H]4F)sc3n2)nn2cc(C)nc12] |
| 321 | 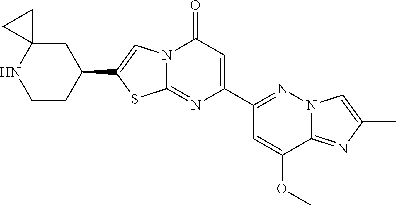 [SMILES: COc1cc(-c2cc(=O)n3cc([C@H]4CCNC5(CC5)C4)sc3n2)nn2cc(C)nc12] |
| 322 | 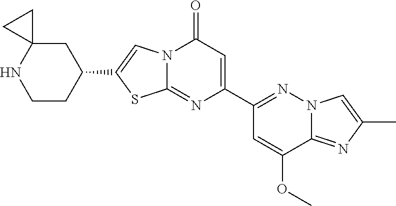 [SMILES: COc1cc(-c2cc(=O)n3cc([C@@H]4CCNC5(CC5)C4)sc3n2)nn2cc(C)nc12] |
| 323 | 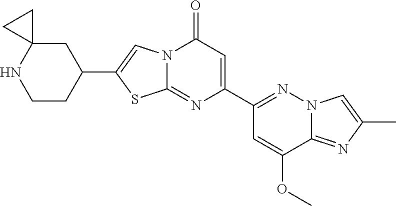 [SMILES: COc1cc(-c2cc(=O)n3cc(C4CCNC5(CC5)C4)sc3n2)nn2cc(C)nc12] |
| 324 | 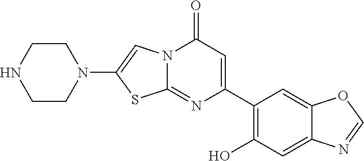 [SMILES: O=c1cc(-c2cc3ocnc3cc2O)nc2sc(N3CCNCC3)cn12] |
| 325 | 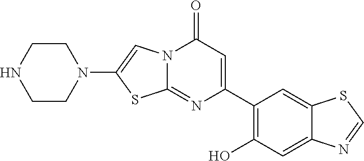 [SMILES: O=c1cc(-c2cc3scnc3cc2O)nc2sc(N3CCNCC3)cn12] |
| 326 | 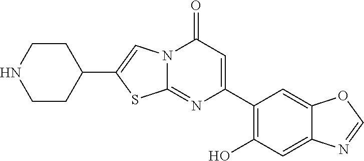 [SMILES: O=c1cc(-c2cc3ocnc3cc2O)nc2sc(C3CCNCC3)cn12] |
| 327 | 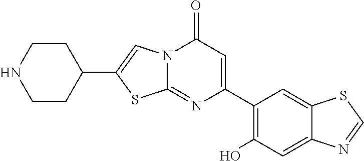 [SMILES: O=c1cc(-c2cc3scnc3cc2O)nc2sc(C3CCNCC3)cn12] |
| 328 | 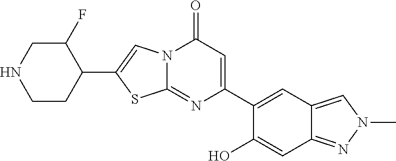 [SMILES: Cn1cc2cc(-c3cc(=O)n4cc(C5CCNCC5F)sc4n3)c(O)cc2n1] |
| 329 | 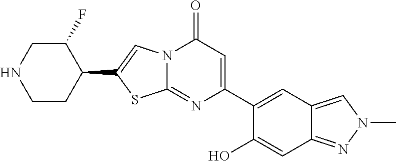 [SMILES: Cn1cc2cc(-c3cc(=O)n4cc([C@H]5CCNC[C@@H]5F)sc4n3)c(O)cc2n1] |
| 330 | 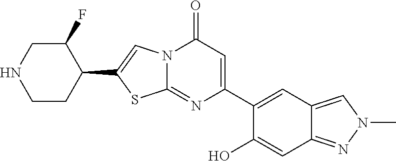 [SMILES: Cn1cc2cc(-c3cc(=O)n4cc([C@H]5CCNC[C@H]5F)sc4n3)c(O)cc2n1] |
| 331 | 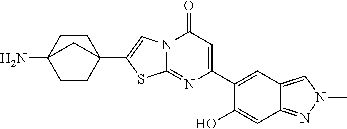 [SMILES: Cn1cc2cc(-c3cc(=O)n4cc(C56CCC(N)(CC5)C6)sc4n3)c(O)cc2n1] |
| 332 | 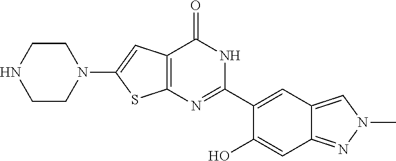 [SMILES: Cn1cc2cc(-c3nc4sc(N5CCNCC5)cc4c(=O)[nH]3)c(O)cc2n1] |
| 333 | 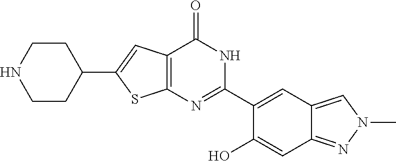 [SMILES: Cn1cc2cc(-c3nc4sc(C5CCNCC5)cc4c(=O)[nH]3)c(O)cc2n1] |

In some embodiments, for Formula (I), A is monocyclic heterocyclyl (e.g., N-methyl piperidinyl); B is bicyclic heteroaryl (e.g., 2-methyl-2H-indazolyl); L¹ and L2 are absent; X is N; Y is C(R^{5b}) (e.g., CH); Z is C(R⁶) (e.g., CH); and R² is absent. In some embodiments, the compound of Formulas (I) and (I-c) is Compound 100, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 2,7-dimethyl-2H-indazolyl); L¹ and L2 are absent; X is N; Y is C(R^{5b}) (e.g., CH); Z is C(R⁶) (e.g., CH); and R² is absent. In some embodiments, the compound of Formulas (I) and (I-c) is Compound 101, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 7-fluoro-2-methyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 102, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 8-fluoro-2-methylimidazo[1,2-a]pyridinyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 103, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2-methyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 104, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,7-dimethyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 105, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 7-fluoro-2-methyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 106, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 107, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 2-methyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 108, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 2,7-dimethyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 109, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 7-fluoro-2-methyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 110, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 8-fluoro-2-methylimidazo[1,2-a]pyridinyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 111, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 2-methyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 112, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 2,7-dimethyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 113, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 7-fluoro-2-methyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 114, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 8-fluoro-2-methylimidazo[1,2-a]pyridinyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 115, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 2-methyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 116, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 2,7-dimethyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 117, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 7-fluoro-2-methyl-2H-indazolyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 118, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 119, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2-methyl-2H-indazolyl); L1 and L2 are —N(R³)—, (e.g. —N(CH₃)—); X is N; Y is C(R^{5b}) (e.g., CH); Z is C(R⁶) (e.g., CH); and R² is absent. In some embodiments, the compound of Formulas (I) and (I-c) is Compound 120, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,7-dimethyl-2H-indazolyl); L1 is —N(R³)—, (e.g. —N(CH₃)—); L2 is absent; X is N; Y is C(R^{5b}) (e.g., CH); Z is C(R⁶) (e.g., CH); and R² is absent. In some embodiments, the compound of Formulas (I) and (I-c) is Compound 121, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 7-fluoro-2-methyl-2H-indazolyl); L1 and L2 are —N(R³)—, (e.g. —N(CH₃)—); X is N; Y is C(R^{5b}) (e.g., CH); Z is C(R⁶) (e.g., CH); and R² is absent. In some embodiments, the compound of Formulas (I) and (I-c) is Compound 122, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L1 is —N(R³)—, (e.g. —N(CH₃)—); L2 is absent; X is N; Y is C(R^{5b}) (e.g., CH); Z is C(R⁶) (e.g., CH); and R² is absent. In some embodiments, the compound of Formulas (I) and (I-c) is Compound 123, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 2-methyl-2H-indazolyl); L1 and L2 are absent; X is C; Y is N(R^{5a}) (e.g., NH); Z is N; and R² is absent. In some embodiments, the compound of Formulas (I) (I-a), and (I-b) is Compound 124, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 2,7-dimethyl-2H-indazolyl); L1 and L2 are absent; X is C; Y is N(R^{5a}) (e.g., NH); Z is N; and R² is absent. In some embodiments, the compound of Formulas (I), (I-a), and (I-b) is Compound 125, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 7-fluoro-2-methyl-2H-indazolyl); L1 and L2 are absent; X is C; Y is N(R^{5a}) (e.g., NH); Z is N; and R² is absent. In some embodiments, the compound of Formulas (I), (I-a), and (I-b) is Compound 126, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 8-fluoro-2-methylimidazo[1,2-a]pyridinyl); L1 and L2 are absent; X is C; Y is N(R^{5a}) (e.g., NH); Z is N; and R² is absent. In some embodiments, the compound of Formulas (I), (I-a), and (I-b) is Compound 127, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2-methyl-2H-indazolyl); L1 and L2 are absent; X is C; Y is N(R^{5a}) (e.g., NH); Z is N; and R² is absent. In some embodiments, the compound of Formulas (I) (I-a), and (I-b) is Compound 128, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,7-dimethyl-2H-indazolyl); L1 and L2 are absent; X is C; Y is N(R^{5a}) (e.g., NH); Z is N; and R² is absent. In some embodiments, the compound of Formulas (I), (I-a), and (I-b) is Compound 129, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 7-fluoro-2-methyl-2H-indazolyl); L1 and L2 are absent; X is C; Y is N(R^{5a}) (e.g., NH); Z is N; and R² is absent. In some embodiments, the compound of Formulas (I), (I-a), and (I-b) is Compound 130, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L1 and L2 are absent; X is C; Y is N(R^{5a}) (e.g., NH); Z is N; and R² is absent. In some embodiments, the compound of Formulas (I), (I-a), and (I-b) is Compound 131, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 2-methyl-2H-indazolyl); L1 and L2 are absent; X is C; Y is N(R^{5a}) (e.g., NH); Z is N; and R² is absent. In some embodiments, the compound of Formulas (I) (I-a), and (I-b) is Compound 132, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 2,7-dimethyl-2H-indazolyl); L1 and L2 are absent; X is C; Y is N(R^{5a}) (e.g., NH); Z is N; and R² is absent. In some embodiments, the compound of Formulas (I), (I-a), and (I-b) is Compound 133, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 7-fluoro-2-methyl-2H-indazolyl); L1 and L2 are absent; X is C; Y is N(R^{5a}) (e.g., NH); Z is N; and R² is absent. In some embodiments, the compound of Formulas (I), (I-a), and (I-b) is Compound 134, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 8-fluoro-2-methylimidazo[1,2-a]pyridinyl); L1 and L2 are absent; X is C; Y is N(R^{5a}) (e.g., NH); Z is N; and R² is absent. In some embodiments, the compound of Formulas (I), (I-a), and (I-b) is Compound 135, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 2-methyl-2H-indazolyl); L1 and L2 are absent; X is C; Y is N(R^{5a}) (e.g., NH); Z is N; and R² is absent. In some embodiments, the compound of Formulas (I) (I-a), and (I-b) is Compound 136, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 2,7-dimethyl-2H-indazolyl); L1 and L2 are absent; X is C; Y is N(R^{5a}) (e.g., NH); Z is N; and R² is absent. In some embodiments, the compound of Formulas (I), (I-a), and (I-b) is Compound 137, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 7-fluoro-2-methyl-2H-indazolyl); L1 and L2 are absent; X is C; Y is N(R^{5a}) (e.g., NH); Z is N; and R² is absent. In some embodiments, the compound of Formulas (I), (I-a), and (I-b) is Compound 138, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 8-fluoro-2-methylimidazo[1,2-a]pyridinyl); L1 and L2 are absent; X is C; Y is N(R^{5a}) (e.g., NH); Z is N; and R² is absent. In some embodiments, the compound of Formulas (I), (I-a), and (I-b) is Compound 139, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 2-methyl-2H-indazolyl); L1 and L2 are absent; X is C; Y is N(R^{5a}) (e.g., NH); Z is N; and R² is absent. In some embodiments, the compound of Formulas (I) (I-a), and (I-b) is Compound 140, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 2,7-dimethyl-2H-indazolyl); L1 and L2 are absent; X is C; Y is N(R^{5a}) (e.g., NH); Z is N; and R² is absent. In some embodiments, the compound of Formulas (I), (I-a), and (I-b) is Compound 141, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 7-fluoro-2-methyl-2H-indazolyl L1 and L2 are absent; X is C; Y is N(R^{5a}) (e.g., NH); Z is N; and R² is absent. In some embodiments, the compound of Formulas (I), (I-a), and (I-b) is Compound 142, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L1 and L2 are absent; X is C; Y is N(R^{5a}) (e.g., NH); Z is N; and R² is absent. In some embodiments, the compound of Formulas (I), (I-a), and (I-b) is Compound 143, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2-methyl-2H-indazolyl); L1 is —N(R³)—, (e.g. —N(CH₃)—); L2 is absent; X is C; Y is N(R^{5a}) (e.g., NH); Z is N; and R² is absent. In some embodiments, the compound of Formulas (I) (I-a), and (I-b) is Compound 144, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,7-dimethyl-2H-indazolyl); L1 is —N(R³)—, (e.g. —N(CH₃)—); L2 is absent; X is C; Y is N(R^{5a}) (e.g., NH); Z is N; and R² is absent. In some embodiments, the compound of Formulas (I), (I-a), and (I-b) is Compound 145, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 7-fluoro-2-methyl-2H-indazolyl); L1 is —N(R³)—, (e.g. —N(CH₃)—); L2 is absent; X is C; Y is N(R^{5a}) (e.g., NH); Z is N; and R² is absent. In some embodiments, the compound of Formulas (I), (I-a), and (I-b) is Compound 146, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L1 is —N(R³)—, (e.g. —N(CH₃)—); L2 is absent; X is C; Y is N(R^{5a}) (e.g., NH); Z is N; and R² is absent. In some embodiments, the compound of Formulas (I), (I-a), and (I-b) is Compound 147, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 2-methyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 148, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 2,7-dimethyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 149, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 7-fluoro-2-methyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 150, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 8-fluoro-2-methylimidazo[1,2-a]pyridinyl); L1 and L2 are absent t; X is N; Y is C(R^{5b}) (e.g., CH); Z is N; and R² is absent. In some embodiments, the compound of Formulas (I), (I-a), and (I-c) is Compound 151, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2-methyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 152, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,7-dimethyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 153, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 7-fluoro-2-methyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 154, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L1 and L2 are absent; X is N; Y is C(R^{5b}) (e.g., CH); Z is N; and R² is absent. In some embodiments, the compound of Formulas (I), (I-a), and (I-c) is Compound 155, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 2-methyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 156, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 2,7-dimethyl-2H-indazolyl); L is absent; X is N; Y is C(R^{5b}) (e.g., CH); Z is N; and R² is absent. In some embodiments, the compound of Formulas (I), (I-a), and (I-c) is Compound 157, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 7-fluoro-2-methyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 158, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 8-fluoro-2-methylimidazo[1,2-a]pyridinyl); L1 and L2 are absent; X is N; Y is C(R^{5b}) (e.g., CH); Z is N; and R² is absent. In some embodiments, the compound of Formulas (I), (I-a), and (I-c) is Compound 159, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 2-methyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 160, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 2,7-dimethyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 161, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 7-fluoro-2-methyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 162, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 8-fluoro-2-methylimidazo[1,2-a]pyridinyl); L1 and L2 are absent; X is N; Y is C(R^{5b}) (e.g., CH); Z is N; and R² is absent. In some embodiments, the compound of Formulas (I), (I-a), and (I-c) is Compound 163, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 2-methyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 164, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 2,7-dimethyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 165, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 7-fluoro-2-methyl-2H-indazolyl); L1 and L2 are absent; X is N; Y is C(R^{5b}) (e.g., CH); Z is N; and R² is absent. In some embodiments, the compound of Formulas (I), (I-a), and (I-c) is Compound 166, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L1 and L2 are absent; X is N; Y is C(R^{5b}) (e.g., CH); Z is N; and R² is absent. In some embodiments, the compound of Formulas (I), (I-a), and (I-c) is Compound 167, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2-methyl-2H-indazolyl); L1 is —N(R³)— (e.g. —N(CH₃)—); L2 is absent; X is N; Y is C(R^{5b}) (e.g., CH); Z is N; and R² is absent. In some embodiments, the compound of Formulas (I), (I-a), and (I-c) is Compound 168, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,7-dimethyl-2H-indazolyl); L1 is —N(R³)— (e.g. —N(CH₃)—); X is N; Y is C(R^{5b}) (e.g., CH); Z is N; and R² is absent. In some embodiments, the compound of Formulas (I), (I-a), and (I-c) is Compound 169, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 7-fluoro-2-methyl-2H-indazolyl); L1 is —N(R³)— (e.g. —N(CH₃)—); X is N; Y is C(R^{5b}) (e.g., CH); Z is N; and R² is absent. In some embodiments, the compound of Formulas (I), (I-a), and (I-c) is Compound 170, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L1 is —N(R³)— (e.g. —N(CH₃)—); L2 is absent; X is N; Y is C(R^{5b}) (e.g., CH); Z is N; and R² is absent. In some embodiments, the compound of Formulas (I), (I-a), and (I-c) is Compound 171, or a pharmaceutically acceptable salt, solvate, hydrate, tautomer, or stereoisomer thereof.

In some embodiments, for Formula (I), A is bicyclic heteroaryl (e.g., 2,7-dimethyl-2H-indazolyl); B is monocyclic heterocyclyl (e.g., N-methyl piperazyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 183, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., N-methyl piperazyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 184, or a pharmaceutically acceptable salt, solvate, hydrate, tautomer, or stereoisomer thereof.

In some embodiments, for Formula (I), A is bicyclic heteroaryl (e.g., 2-methyl-2H-indazolyl); B is monocyclic heterocyclyl (e.g., piperidinyl); L1 and L2 are absent; X is C; Y is N(R^{5a}) (e.g., NH); Z is N; and R² is absent. In some embodiments, the compound of Formulas (I) (I-a), and (I-b) is Compound 192, or a pharmaceutically acceptable salt, solvate, hydrate, tautomer, or stereoisomer thereof.

In some embodiments, for Formula (I), A is bicyclic heteroaryl (e.g., 2-methyl-2H-indazolyl); B is monocyclic heterocyclyl (e.g., piperidinyl); L1 and L2 are absent; X is N; Y is C(R^{5b}) (e.g., CH); Z is N; and R² is absent. In some embodiments, the compound of Formulas (I), (I-a), and (I-c) is Compound 193, or a pharmaceutically acceptable salt, solvate, hydrate, tautomer, or stereoisomer thereof.

In some embodiments, for Formula (I), A is bicyclic heteroaryl (e.g., 2-methyl-2H-indazolyl); B is monocyclic heterocyclyl (e.g., piperidinyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 194, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 2,8-dimethylimidazo[1,2-b]pyridazyl); L1 is —N(R³)— (e.g., —N(CH₃)—); L2 is absent; X is N; Y is C(R^{5b}) (e.g., CH); Z is C(R⁶) (e.g., CH); and R² is absent. In some embodiments, the compound of Formulas (I) and (I-c) is Compound 205, or a pharmaceutically acceptable salt, solvate, hydrate, tautomer, or stereoisomer thereof.

In some embodiments, for Formula (I), A is bicyclic heteroaryl (e.g., 2,7-dimethyl-2H-indazolyl); B is monocyclic heterocyclyl (e.g., piperazyl); L1 and L2 are absent; X is N; Y is C(R^{5b}) (e.g., CH); Z is N; and R² is absent. In some embodiments, the compound of Formulas (I), (I-a), and (I-c) is Compound 206, or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 8-fluoro-2-methylimidazo[1,2-a]pyridinyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 207, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is bicyclic heterocyclyl (e.g., 4,7-diazaspiro[2.5]octanyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 208, or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 8-fluoro-2-methylimidazo[1,2-a]pyridinyl); L1 and L2 are absent; X is N; Y is C(R^{5b}) (e.g., CH); Z is N; and R² is absent. In some embodiments, the compound of Formulas (I), (I-a), and (I-c) is Compound 209, or a pharmaceutically acceptable salt, solvate, hydrate, tautomer, or stereoisomer thereof.

In some embodiments, for Formula (I), A is bicyclic heteroaryl (e.g., 2,7-dimethyl-2H-indazolyl); B is monocyclic heterocyclyl (e.g., N-methyl piperazyl); L1 and L2 are absent; X is N; Y is C(R^{5b}) (e.g., CH); Z is N; and R² is absent. In some embodiments, the compound of Formulas (I), (I-a), and (I-c) is Compound 210, or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 2,8-dimethylimidazo[1,2-b]pyridazyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 211, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4,7-diazaspiro[2.5]octanyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 212, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperazyl); L1 and L2 are absent; X is N; Y is C(R^{5b}) (e.g., CH); Z is C(R⁶) (e.g., CH); and R² is absent. In some embodiments, the compound of Formulas (I) and (I-c) is Compound 213, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperazyl); L1 and L2 are absent; X is N; Y is C(R^{5b}) (e.g., CH); Z is N; and R² is absent. In some embodiments, the compound of Formulas (I), (I-a), and (I-c) is Compound 214 or 215, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is bicyclic heterocyclyl (e.g., 4,7-diazaspiro[2.5]octanyl); L1 and L2 are absent; X is N; Y is C(R^{5b}) (e.g., CH); Z is N; and R² is absent. In some embodiments, the compound of Formulas (I), (I-a), and (I-c) is Compound 216, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., N-methyl piperazyl); L1 and L2 are absent; X is N; Y is C(R^{5b}) (e.g., CH); Z is N; and R² is absent. In some embodiments, the compound of Formulas (I), (I-a), and (I-c) is Compound 217, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is bicyclic heterocyclyl (e.g., 4-methyl-4,7-diazaspiro[2.5]octanyl); L1 and L2 are absent; X is N; Y is C(R^{5b}) (e.g., CH); Z is N; and R² is absent. In some embodiments, the compound of Formulas (I), (I-a), and (I-c) is Compound 218, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., 8-fluoro-2-methylimidazo[1,2-a]pyridinyl); L1 and L2 are absent; X is C; Y is N(R^{5a}) (e.g., NH); Z is C(R⁶) (e.g., CH); and R² is absent. In some embodiments, the compound of Formulas (I) and (I-b) is Compound 250, or a pharmaceutically acceptable salt, solvate, hydrate, tautomer, or stereoisomer thereof.

In some embodiments, for Formula (I), A is monocyclic heterocyclyl (e.g., N-methyl piperazyl); B is a bicyclic heteroaryl (e.g., 4,6-dimethylpyrazolo[1,5-a]pyrazinyl); L1 and L2 are absent; X is C(R⁶) (e.g., CH); Y is N; Z is N; and R² is absent. In some embodiments, the compound of Formulas (I) and (I-b) is Compound 267, or a pharmaceutically acceptable salt, solvate, hydrate, tautomer, or stereoisomer thereof.

In some embodiments, for Formula (I), A is monocyclic heterocyclyl (e.g., piperazine); B is a bicyclic heteroaryl (e.g., 4,6-dimethylpyrazolo[1,5-a]pyrazinyl); L1 and L2 are absent; X is C(R⁶) (e.g., CH); Y is N; Z is N; and R² is absent. In some embodiments, the compound of Formulas (I) and (I-b) is Compound 268, or a pharmaceutically acceptable salt, solvate, hydrate, tautomer, or stereoisomer thereof.

In some embodiments the compound of Formula (II) is a compound of Formula (II-a):

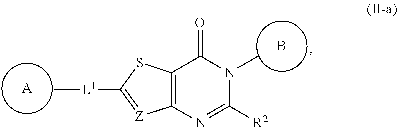

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; Z is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R¹; R² is absent, hydrogen, or C₁-C₆-alkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁶ is hydrogen, C₁-C₆-alkyl, C₁-C₆-heteroalkyl, C₁-C₆-haloalkyl, or halo; R⁷ is hydrogen, C₁-C₆-alkyl, C₁-C₆-heteroalkyl, C₁-C₆-haloalkyl, halo, or —OR^{A}; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁹; each R⁹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl.

In some embodiments, A is selected from

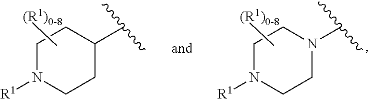

wherein R¹ is as defined herein.

In some embodiments, A is selected from

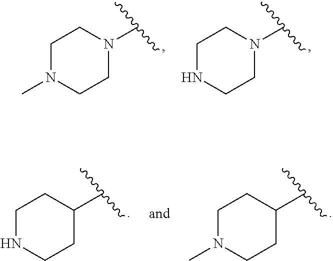

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

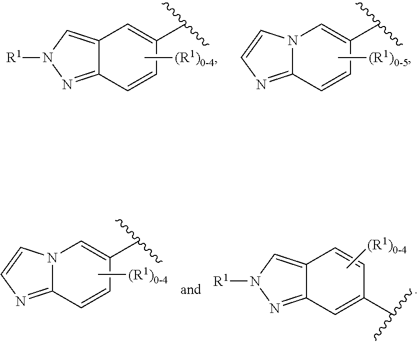

In some embodiments, A is

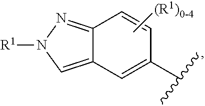

wherein R¹ is as defined herein.

In some embodiments, A is selected from

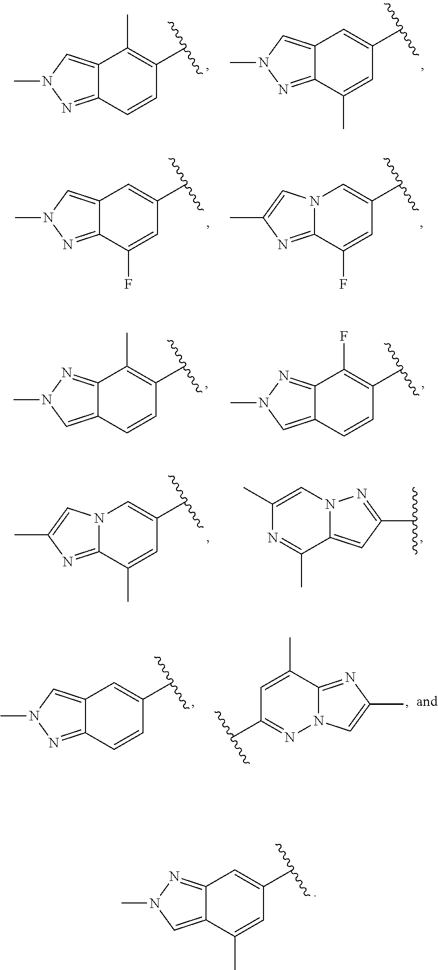

In some embodiments A is

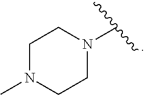 [SMILES: *N1CCN(C)CC1]

In some embodiments, A is

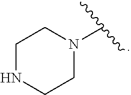 [SMILES: *N1CCNCC1]

In some embodiments, A is

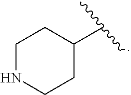 [SMILES: *C1CCNCC1]

In some embodiments, A is

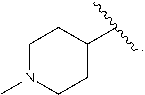 [SMILES: *C1CCN(C)CC1]

In some embodiments, A is

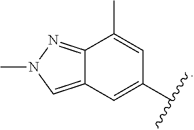 [SMILES: *c1cc(C)c2nn(C)cc2c1]

In some embodiments, A is

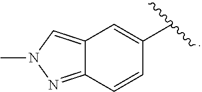 [SMILES: *c1ccc2nn(C)cc2c1]

In some embodiments A is

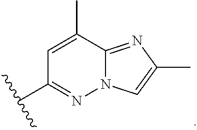 [SMILES: *c1cc(C)c2nc(C)cn2n1]

In some embodiments, A is

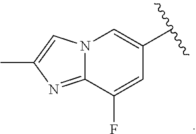 [SMILES: *c1cc(F)c2nc(C)cn2c1]

In some embodiments, A is

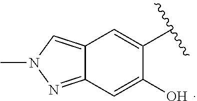 [SMILES: *c1cc2cn(C)nc2cc1O]

In some embodiments, A is

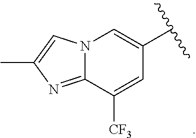 [SMILES: *c1cc(C(F)(F)F)c2nc(C)cn2c1]

In some embodiments, B is heteroaryl optionally substituted with one or more R¹. In some embodiments, B is nitrogen-containing heteroaryl. In some embodiments, B is bicyclic nitrogen-containing heteroaryl. In some embodiments, B is optionally substituted indazolyl. In some embodiments, B is selected from

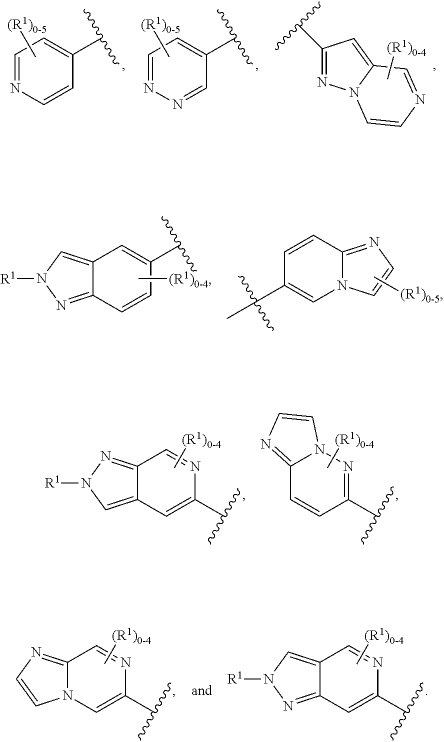

In some embodiments, B is selected from

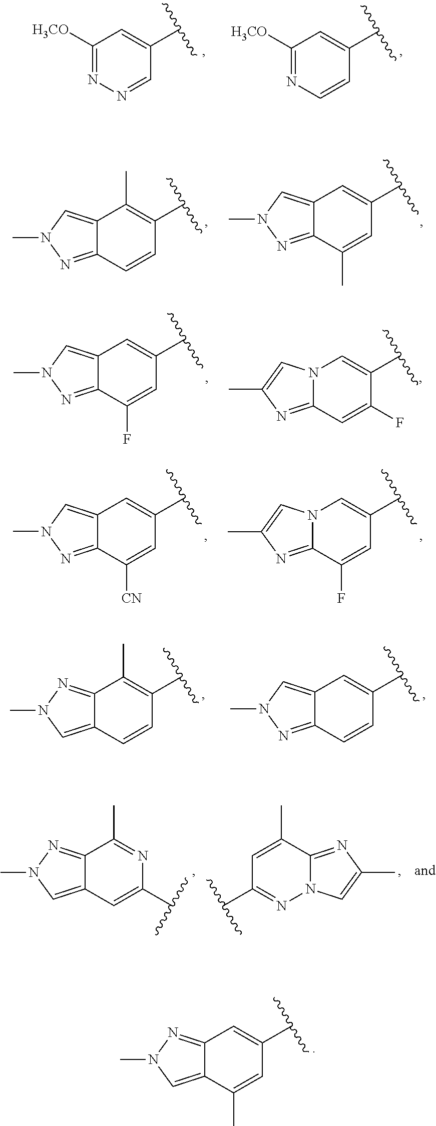

In some embodiments, B is heterocyclyl optionally substituted with one or more R¹. In some embodiments, B is monocyclic nitrogen-containing heterocyclyl. In some embodiments, B is selected from

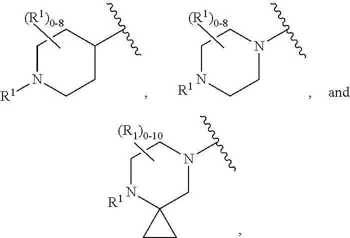

wherein R¹ is as defined herein.

In some embodiments, B is selected from

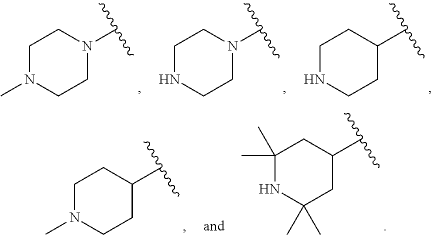

In some embodiments, B is

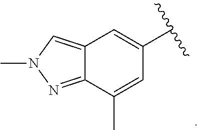 [SMILES: *c1cc(C)c2nn(C)cc2c1]

In some embodiments, B is

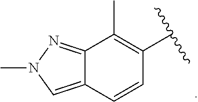 [SMILES: *c1ccc2cn(C)nc2c1C]

In some embodiments, B is

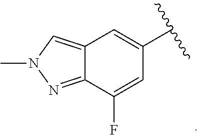 [SMILES: *c1cc(F)c2nn(C)cc2c1]

In some embodiments, B is

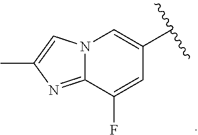 [SMILES: *c1cc(F)c2nc(C)cn2c1]

In some embodiments, B is

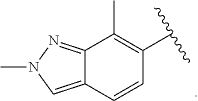 [SMILES: *c1ccc2cn(C)nc2c1C]

In some embodiments, B is

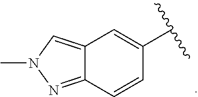 [SMILES: *c1ccc2nn(C)cc2c1]

In some embodiments, B is

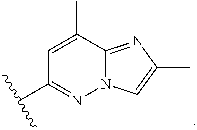 [SMILES: *c1cc(C)c2nc(C)cn2n1]

In some embodiments, B is

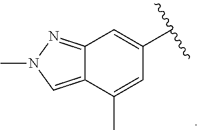 [SMILES: *c1cc(C)c2cn(C)nc2c1]

In some embodiments, B is

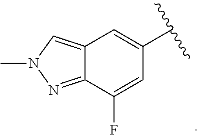 [SMILES: *c1cc(F)c2nn(C)cc2c1]

In some embodiments, B is

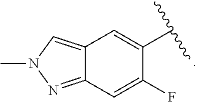 [SMILES: *c1cc2cn(C)nc2cc1F]

In some embodiments, B is

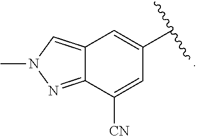 [SMILES: *c1cc(C#N)c2nn(C)cc2c1]

In some embodiments, B is

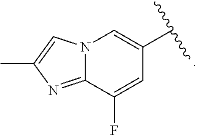 [SMILES: *c1cc(F)c2nc(C)cn2c1]

In some embodiments, B is

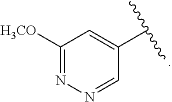 [SMILES: *c1cnnc(OC)c1]

In some embodiments, B is

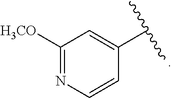 [SMILES: *c1ccnc(OC)c1]

In some embodiments, B is

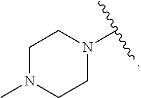 [SMILES: *N1CCN(C)CC1]

In some embodiments, B is

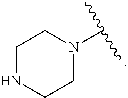 [SMILES: *N1CCNCC1]

In some embodiments, B is

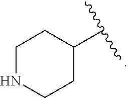 [SMILES: *C1CCNCC1]

In some embodiments, B is

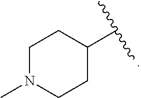 [SMILES: *C1CCN(C)CC1]

In some embodiments, B is

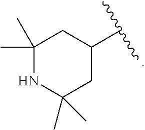 [SMILES: *C1CC(C)(C)NC(C)(C)C1]

In some embodiments, L¹ is absent or C₁-C₆-heteroalkylene (e.g., —N(CH₃)—). In some embodiments, L¹ is absent. In some embodiments, L¹ is C₁-C₆-heteroalkylene (e.g., —N(CH₃)—).

In some embodiments, Z is N. In some embodiments, Z is C(R⁶) (e.g., CH).

In some embodiments, the compound of Formula (II) is a compound of Formula (II-b):

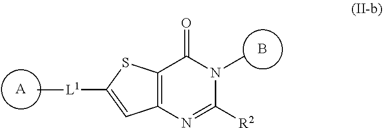

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R¹; R² is absent, hydrogen, or C₁-C₆-alkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁷ is hydrogen, C₁-C₆-alkyl, C₁-C₆-heteroalkyl, C₁-C₆-haloalkyl, halo, or —OR^{A}; each R^{A} is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁹; each R⁹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl.

In some embodiments, A is selected from

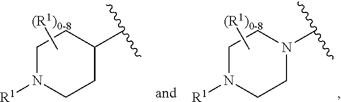

wherein R¹ is as defined herein. In some embodiments, A is selected from

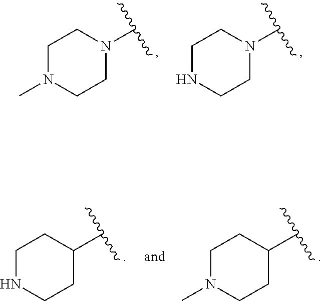

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

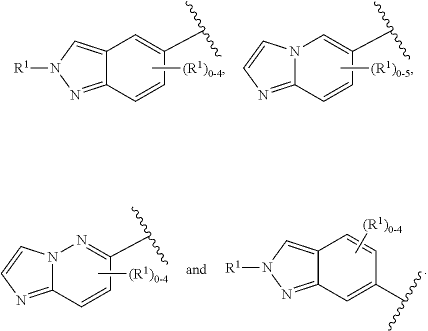

In some embodiments, A is

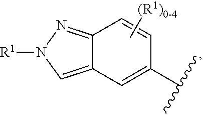

wherein R¹ is as defined herein.

In some embodiments, A is selected from

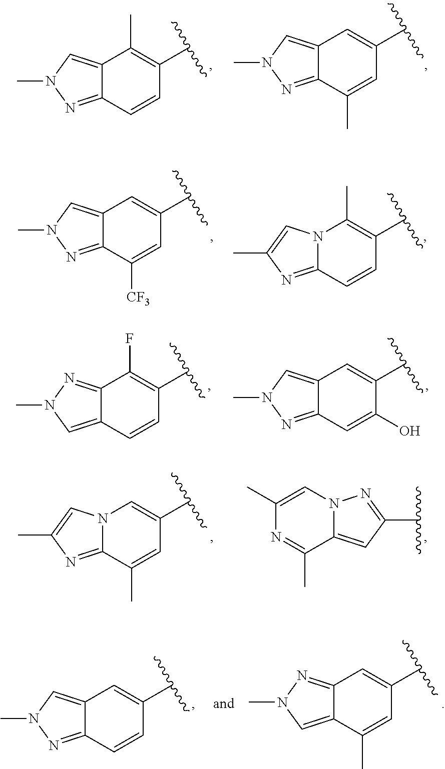

In some embodiments, A is

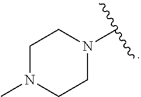 [SMILES: *N1CCN(C)CC1]

In some embodiments A is

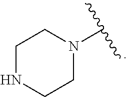 [SMILES: *N1CCNCC1]

In some embodiments, A is

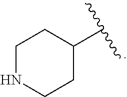 [SMILES: *C1CCNCC1]

In some embodiments, A is

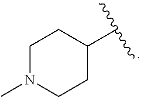 [SMILES: *C1CCN(C)CC1]

In some embodiments, A is

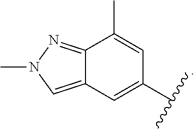 [SMILES: *c1cc(C)c2nn(C)cc2c1]

In some embodiments, A is

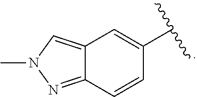 [SMILES: *c1ccc2nn(C)cc2c1]

In some embodiments, A is

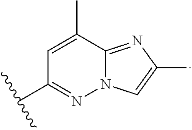

In some embodiments, A is

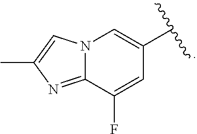 [SMILES: *c1cc(F)c2nc(C)cn2c1]

In some embodiments, A

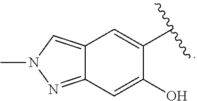 [SMILES: *c1cc2cn(C)nc2cc1O]

In some embodiments, A is

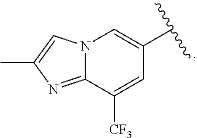 [SMILES: *c1cc(C(F)(F)F)c2nc(C)cn2c1]

In some embodiments, B is heteroaryl optionally substituted with one or more R¹. In some embodiments, B is nitrogen-containing heteroaryl. In some embodiments, B is bicyclic nitrogen-containing heteroaryl. In some embodiments, B is optionally substituted indazolyl. In some embodiments, B is selected from

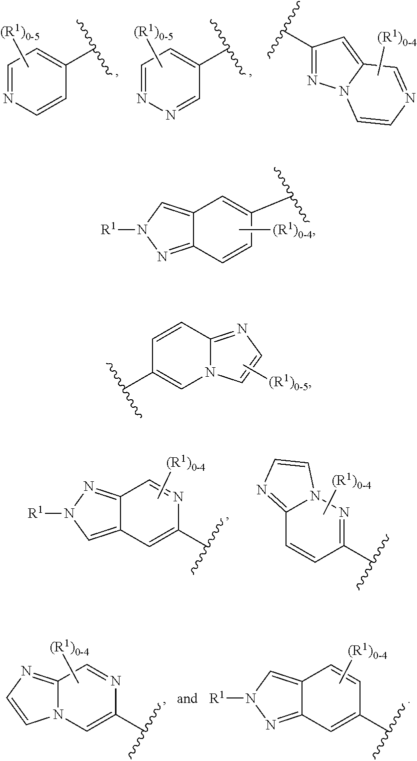

In some embodiments, B is selected from

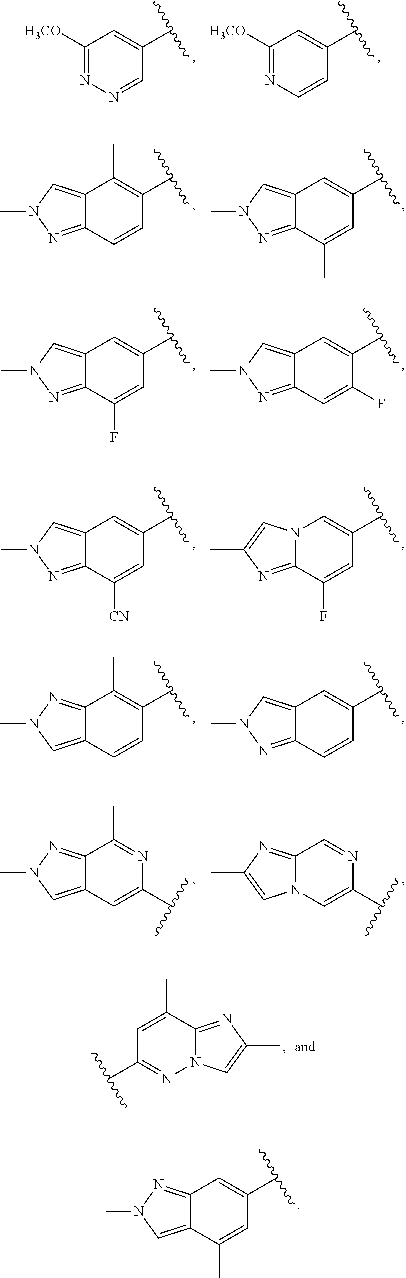

In some embodiments, B is heterocyclyl optionally substituted with one or more R¹. In some embodiments, B is monocyclic nitrogen-containing heterocyclyl. In some embodiments, B is selected from

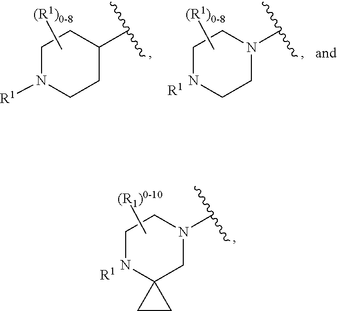

wherein R¹ is as defined herein.

In some embodiments, B is selected from

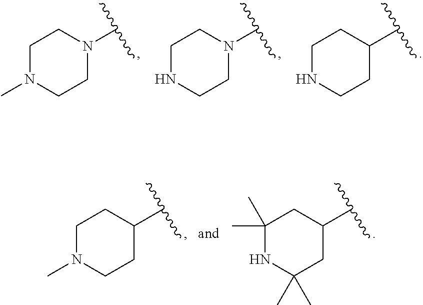

In some embodiments, B is

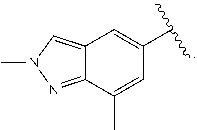 [SMILES: *c1cc(C)c2nn(C)cc2c1]

In some embodiments, B is

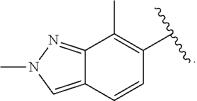 [SMILES: *c1ccc2cn(C)nc2c1C]

In some embodiments, B is

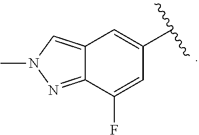 [SMILES: *c1cc(F)c2nn(C)cc2c1]

In some embodiments, B is

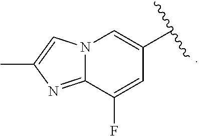 [SMILES: *c1cc(F)c2nc(C)cn2c1]

In some embodiments, B is

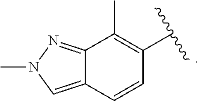 [SMILES: *c1ccc2cn(C)nc2c1C]

In some embodiments, B is

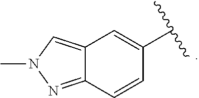 [SMILES: *c1ccc2nn(C)cc2c1]

In some embodiments, B is

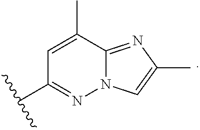 [SMILES: *c1cc(C)c2nc(C)cn2n1]

In some embodiments, B is

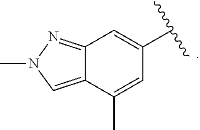 [SMILES: *c1cc(C)c2cn(C)nc2c1]

In some embodiments, B is

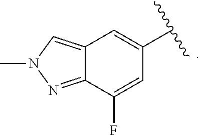 [SMILES: *c1cc(F)c2nn(C)cc2c1]

In some embodiments, B is

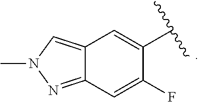 [SMILES: *c1cc2cn(C)nc2cc1F]

In some embodiments, B is

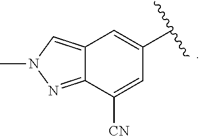 [SMILES: *c1cc(C#N)c2nn(C)cc2c1]

In some embodiments, B is

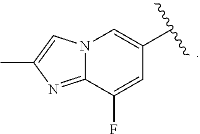 [SMILES: *c1cc(F)c2nc(C)cn2c1]

In some embodiments, B is

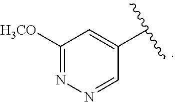 [SMILES: *c1cnnc(OC)c1]

In some embodiments, B is

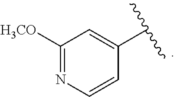 [SMILES: *c1ccnc(OC)c1]

In some embodiments, B is

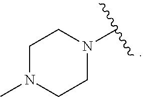 [SMILES: *N1CCN(C)CC1]

In some embodiments, B is

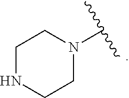 [SMILES: *N1CCNCC1]

In some embodiments, B is

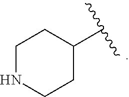 [SMILES: *C1CCNCC1]

In some embodiments, B is

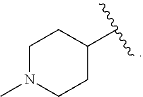 [SMILES: *C1CCN(C)CC1]

In some embodiments, B is

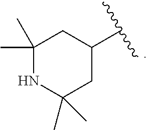 [SMILES: *C1CC(C)(C)NC(C)(C)C1]

In some embodiments, L¹ is absent or C₁-C₆-heteroalkylene (e.g., —N(CH₃)—). In some embodiments, L¹ is absent. In some embodiments, L¹ is C₁-C₆-heteroalkylene (e.g., —N(CH₃)—).

In some embodiments, the compound of Formula (II) is a compound of Formula (II-c):

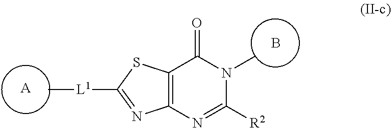

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁷ is hydrogen, C₁-C₆-alkyl, C₁-C₆-heteroalkyl, C₁-C₆-haloalkyl, halo, or —OR^{A}; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁹; each R⁹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl.

In some embodiments, A is selected from

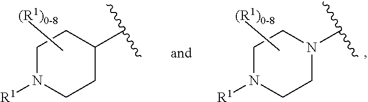

wherein R¹ as defined herein.

In some embodiments, A is selected from

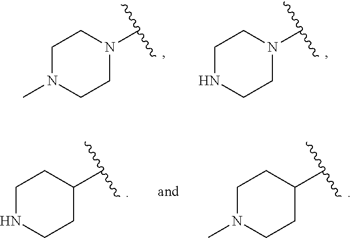

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

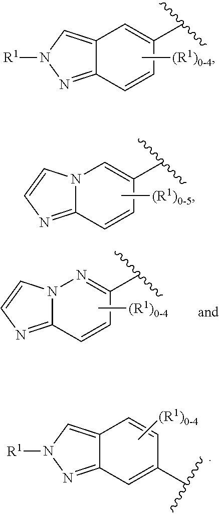

In some embodiments, A is

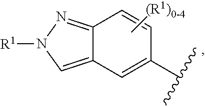

wherein R¹ is as defined herein.

In some embodiments, A is selected from

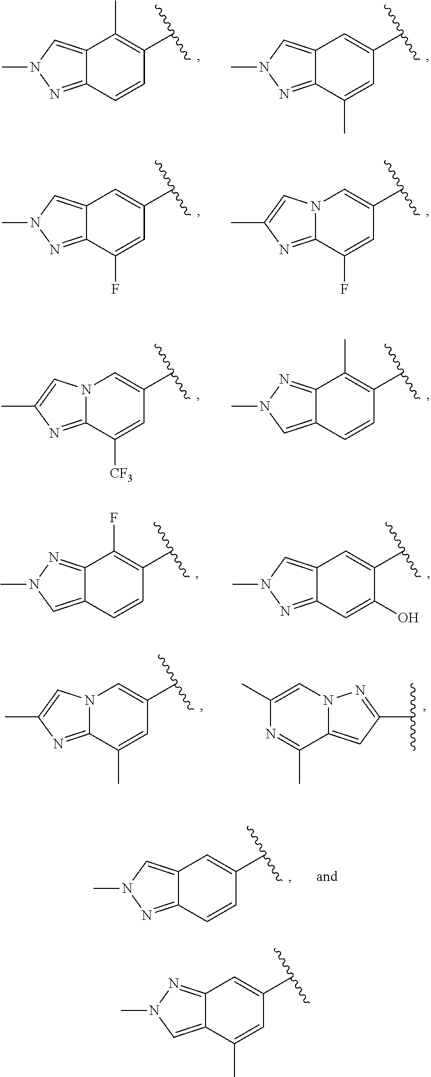

In some embodiments, A is

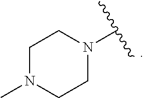 [SMILES: *N1CCN(C)CC1]

In some embodiments, A is

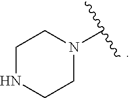 [SMILES: *N1CCNCC1]

In some embodiments, A is

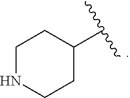 [SMILES: *C1CCNCC1]

In some embodiments, A is

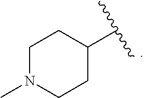 [SMILES: *C1CCN(C)CC1]

In some embodiments, A is

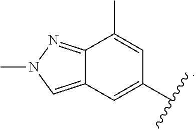 [SMILES: *c1cc(C)c2nn(C)cc2c1]

In some embodiments, A is

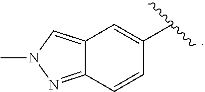 [SMILES: *c1ccc2nn(C)cc2c1]

In some embodiments, A is

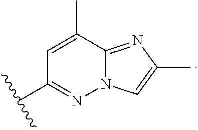 [SMILES: *c1cc(C)c2nc(C)cn2n1]

In some embodiments, A is

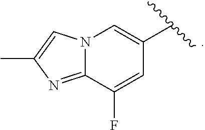 [SMILES: *c1cc(F)c2nc(C)cn2c1]

In some embodiments, A is

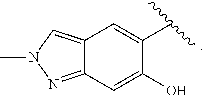 [SMILES: *c1cc2cn(C)nc2cc1O]

In some embodiments, A is

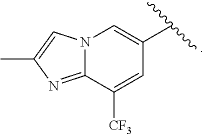 [SMILES: *c1cc(C(F)(F)F)c2nc(C)cn2c1]

In some embodiments, B is heteroaryl optionally substituted with one or more R¹. In some embodiments, B is nitrogen-containing heteroaryl. In some embodiments, B is bicyclic nitrogen-containing heteroaryl. In some embodiments, B is optionally substituted indazolyl. In some embodiments, B is selected from

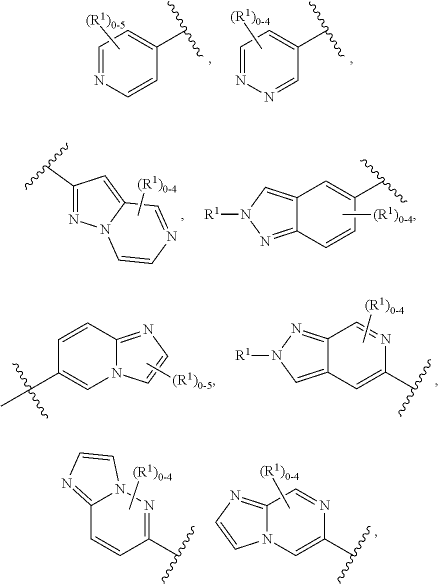

In some embodiments, B is selected from

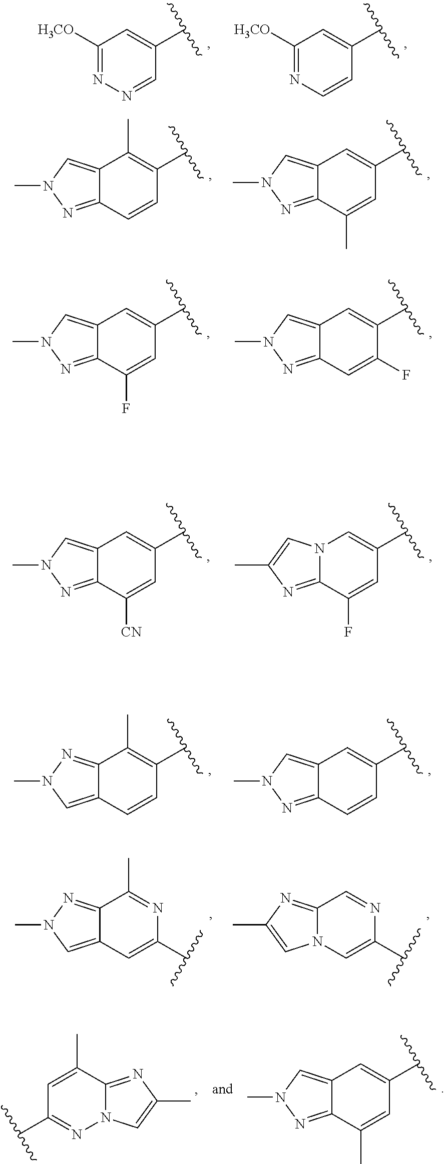

In some embodiments, B is heterocyclyl optionally substituted with one or more R¹. In some embodiments, B is monocyclic nitrogen-containing heterocyclyl. In some embodiments, B is selected from

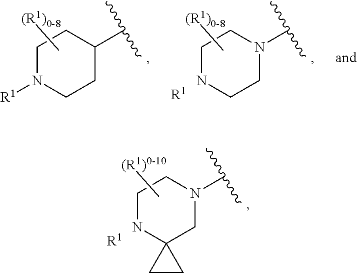

wherein R¹ is as defined herein.

In some embodiments, B is selected from

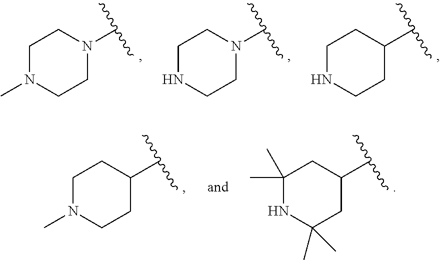

In some embodiments B is

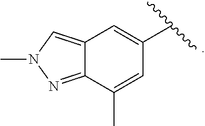 [SMILES: *c1cc(C)c2nn(C)cc2c1]

In some embodiments, B is

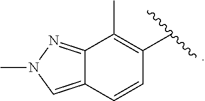 [SMILES: *c1ccc2cn(C)nc2c1C]

In some embodiments, B is

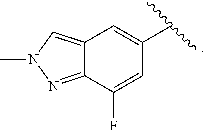 [SMILES: *c1cc(F)c2nn(C)cc2c1]

In some embodiments, B is

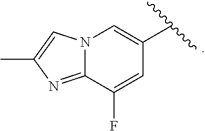 [SMILES: *c1cc(F)c2nc(C)cn2c1]

In some embodiments, B is

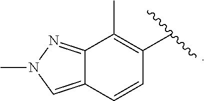 [SMILES: *c1ccc2cn(C)nc2c1C]

In some embodiments, B is

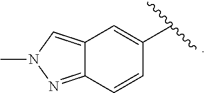 [SMILES: *c1ccc2nn(C)cc2c1]

In some embodiments, B is

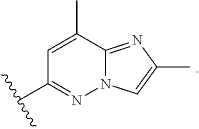 [SMILES: *c1cc(C)c2nc(C)cn2n1]

In some embodiments, B is

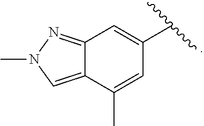 [SMILES: *c1cc(C)c2cn(C)nc2c1]

In some embodiments, B is

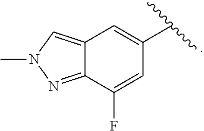 [SMILES: *c1cc(F)c2nn(C)cc2c1]

In some embodiments, B is

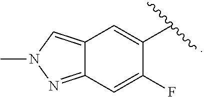 [SMILES: *c1cc2cn(C)nc2cc1F]

In some embodiments, B is

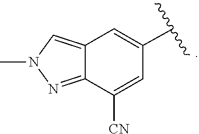 [SMILES: *c1cc(C#N)c2nn(C)cc2c1]

In some embodiments, B is

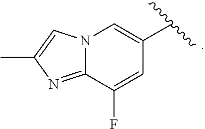 [SMILES: *c1cc(F)c2nc(C)cn2c1]

In some embodiments, B is

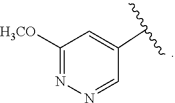 [SMILES: *c1cnnc(OC)c1]

In some embodiments, B is

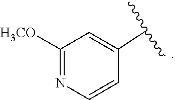 [SMILES: *c1ccnc(OC)c1]

In some embodiments, B is

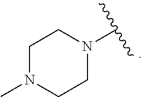 [SMILES: *N1CCN(C)CC1]

In some embodiments, B is

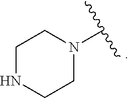 [SMILES: *N1CCNCC1]

In some embodiments, B is

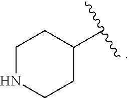 [SMILES: *C1CCNCC1]

In some embodiments, B is

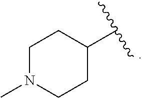 [SMILES: *C1CCN(C)CC1]

In some embodiments, B is

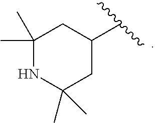 [SMILES: *C1CC(C)(C)NC(C)(C)C1]

In some embodiments, L¹ is absent or C₁-C₆-heteroalkylene (e.g., —N(CH₃)—). In some embodiments, L¹ is absent. In some embodiments, L¹ is C₁-C₆-heteroalkylene (e.g., —N(CH₃)—).

In some embodiments, Z is N. In some embodiments, Z is C(R⁶) (e.g., CH). In some embodiments, R⁷ is hydrogen.

In some embodiments, the compound of Formula (II) is selected from a compound in Table 2, or a pharmaceutically acceptable salt, solvate, hydrate, tautomer, or stereoisomer thereof.

TABLE 2
Exemplary compounds of Formula (II).

| Cmpnd | |
|---|---|
| No. | Structure |
| 182 | 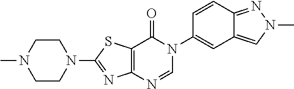 |
| 203 | 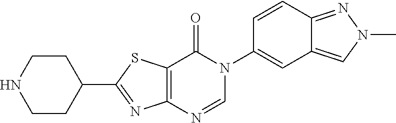 [SMILES: Cn1cc2cc(-n3cnc4nc(C5CCNCC5)sc4c3=O)ccc2n1] |
| 204 | 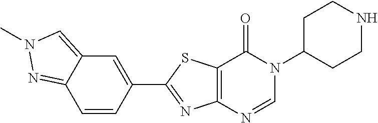 [SMILES: Cn1cc2cc(-c3nc4ncn(C5CCNCC5)c(=O)c4s3)ccc2n1] |
| 225 | 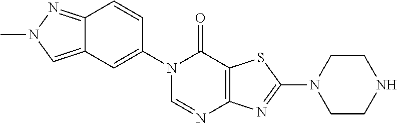 [SMILES: Cn1cc2cc(-n3cnc4nc(N5CCNCC5)sc4c3=O)ccc2n1] |
| 226 | 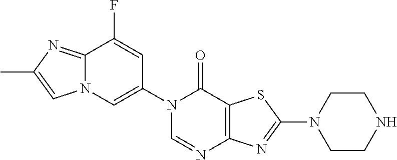 [SMILES: Cc1cn2cc(-n3cnc4nc(N5CCNCC5)sc4c3=O)cc(F)c2n1] |
| 227 | 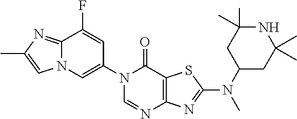 |
| 228 | 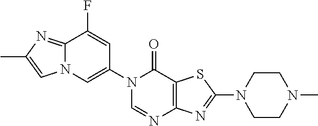 |
| 229 | 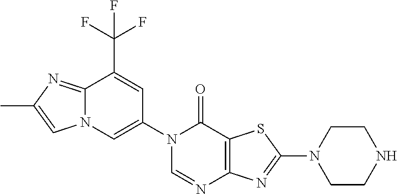 [SMILES: Cc1cn2cc(-n3cnc4nc(N5CCNCC5)sc4c3=O)cc(C(F)(F)F)c2n1] |
| 230 | 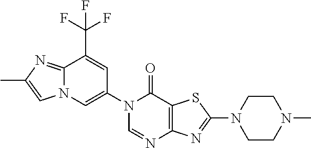 |
| 231 | 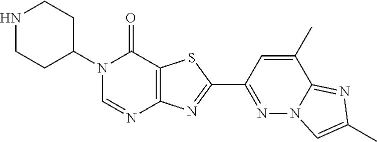 [SMILES: Cc1cn2nc(-c3nc4ncn(C5CCNCC5)c(=O)c4s3)cc(C)c2n1] |
| 232 | 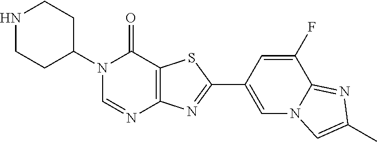 [SMILES: Cc1cn2cc(-c3nc4ncn(C5CCNCC5)c(=O)c4s3)cc(F)c2n1] |
| 233 | 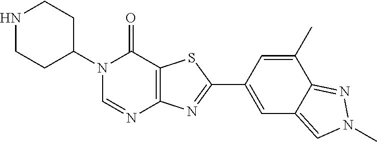 [SMILES: Cc1cc(-c2nc3ncn(C4CCNCC4)c(=O)c3s2)cc2cn(C)nc12] |
| 234 | 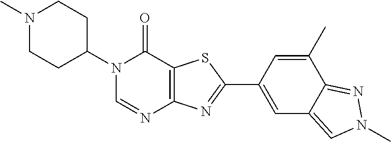 |
| 235 | 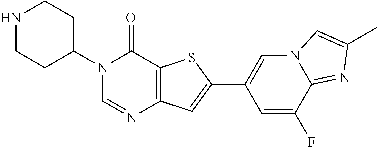 [SMILES: Cc1cn2cc(-c3cc4ncn(C5CCNCC5)c(=O)c4s3)cc(F)c2n1] |
| 236 | 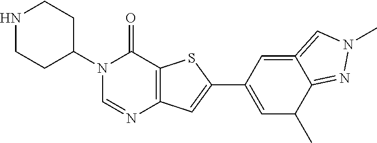 |
| 237 | 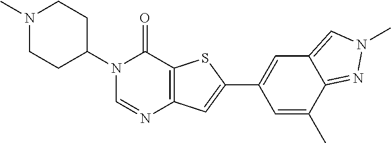 |
| 238 | 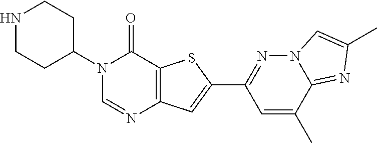 [SMILES: Cc1cn2nc(-c3cc4ncn(C5CCNCC5)c(=O)c4s3)cc(C)c2n1] |
| 239 | 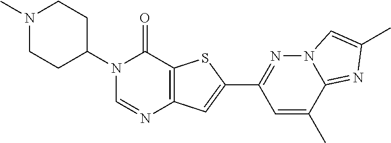 |
| 251 | 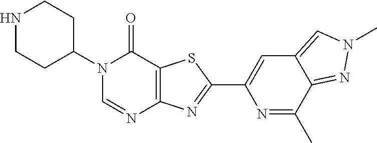 [SMILES: Cc1nc(-c2nc3ncn(C4CCNCC4)c(=O)c3s2)cc2cn(C)nc12] |
| 252 | 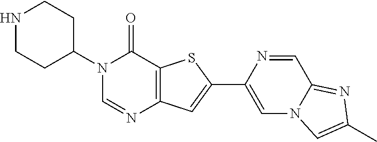 [SMILES: Cc1cn2cc(-c3cc4ncn(C5CCNCC5)c(=O)c4s3)ncc2n1] |
| 253 | 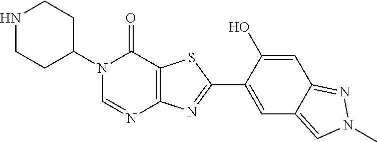 [SMILES: Cn1cc2cc(-c3nc4ncn(C5CCNCC5)c(=O)c4s3)c(O)cc2n1] |
| 257 | 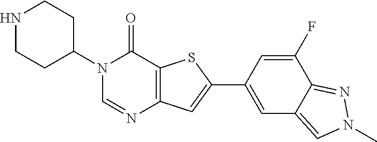 [SMILES: Cn1cc2cc(-c3cc4ncn(C5CCNCC5)c(=O)c4s3)cc(F)c2n1] |
| 258 | 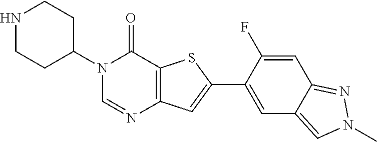 [SMILES: Cn1cc2cc(-c3cc4ncn(C5CCNCC5)c(=O)c4s3)c(F)cc2n1] |
| 259 | 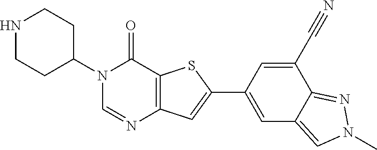 [SMILES: Cn1cc2cc(-c3cc4ncn(C5CCNCC5)c(=O)c4s3)cc(C#N)c2n1] |
| 260 | 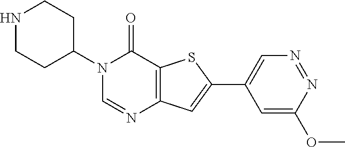 [SMILES: COc1cc(-c2cc3ncn(C4CCNCC4)c(=O)c3s2)cnn1] |
| 261 | 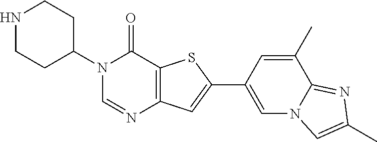 [SMILES: Cc1cn2cc(-c3cc4ncn(C5CCNCC5)c(=O)c4s3)cc(C)c2n1] |
| 262 | 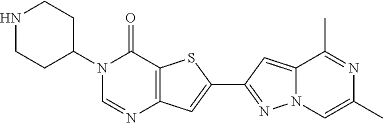 [SMILES: Cc1cn2nc(-c3cc4ncn(C5CCNCC5)c(=O)c4s3)cc2c(C)n1] |
| 263 | 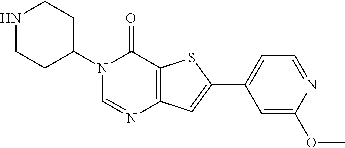 [SMILES: COc1cc(-c2cc3ncn(C4CCNCC4)c(=O)c3s2)ccn1] |
| 264 | 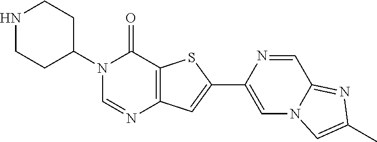 [SMILES: Cc1cn2cc(-c3cc4ncn(C5CCNCC5)c(=O)c4s3)ncc2n1] |
| 265 | 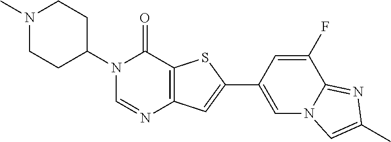 |
| 269 | 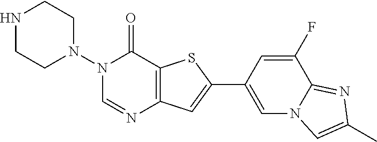 [SMILES: Cc1cn2cc(-c3cc4ncn(N5CCNCC5)c(=O)c4s3)cc(F)c2n1] |
| 270 | 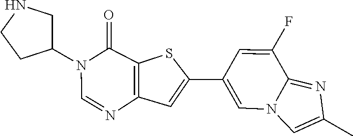 [SMILES: Cc1cn2cc(-c3cc4ncn(C5CCNC5)c(=O)c4s3)cc(F)c2n1] |
| 271 | 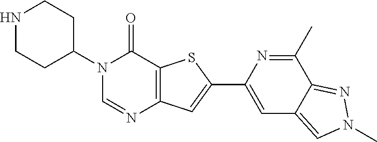 [SMILES: Cc1nc(-c2cc3ncn(C4CCNCC4)c(=O)c3s2)cc2cn(C)nc12] |
| 272 | 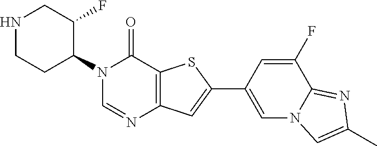 [SMILES: Cc1cn2cc(-c3cc4ncn([C@H]5CCNC[C@@H]5F)c(=O)c4s3)cc(F)c2n1] |
| 273 | 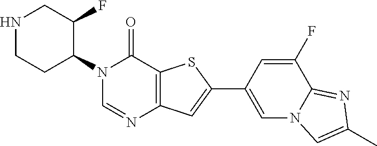 [SMILES: Cc1cn2cc(-c3cc4ncn([C@H]5CCNC[C@H]5F)c(=O)c4s3)cc(F)c2n1] |
| 274 | 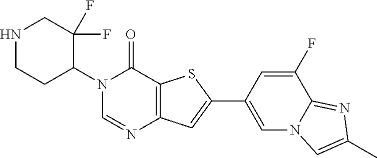 [SMILES: Cc1cn2cc(-c3cc4ncn(C5CCNCC5(F)F)c(=O)c4s3)cc(F)c2n1] |
| 275 | 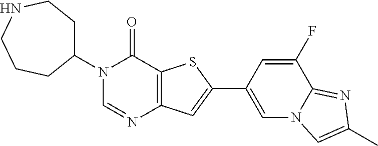 [SMILES: Cc1cn2cc(-c3cc4ncn(C5CCCNCC5)c(=O)c4s3)cc(F)c2n1] |
| 276 | 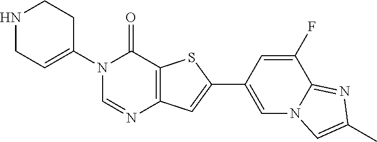 [SMILES: Cc1cn2cc(-c3cc4ncn(C5=CCNCC5)c(=O)c4s3)cc(F)c2n1] |
| 277 | 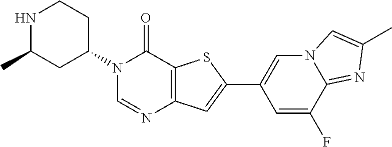 |
| 278 | 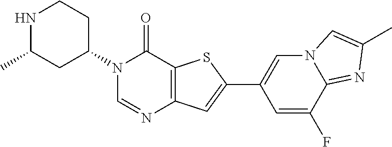 |
| 279 | 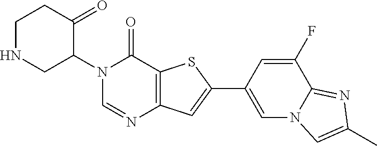 [SMILES: Cc1cn2cc(-c3cc4ncn(C5CNCCC5=O)c(=O)c4s3)cc(F)c2n1] |
| 280 | 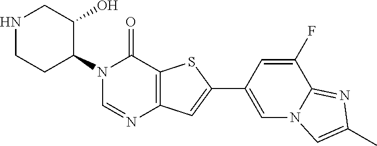 [SMILES: Cc1cn2cc(-c3cc4ncn([C@H]5CCNC[C@@H]5O)c(=O)c4s3)cc(F)c2n1] |
| 281 | 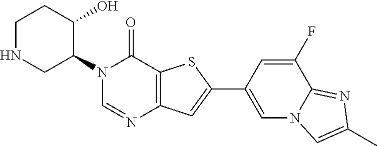 [SMILES: Cc1cn2cc(-c3cc4ncn([C@H]5CNCC[C@@H]5O)c(=O)c4s3)cc(F)c2n1] |
| 334 | 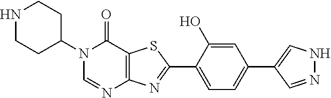 [SMILES: O=c1c2sc(-c3ccc(-c4cn[nH]c4)cc3O)nc2ncn1C1CCNCC1] |
| 335 | 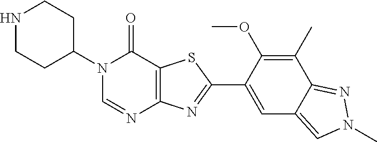 [SMILES: COc1c(-c2nc3ncn(C4CCNCC4)c(=O)c3s2)cc2cn(C)nc2c1C] |
| 336 | 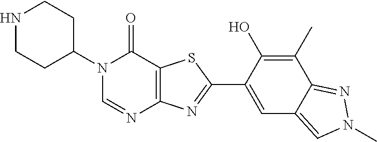 [SMILES: Cc1c(O)c(-c2nc3ncn(C4CCNCC4)c(=O)c3s2)cc2cn(C)nc12] |

In some embodiments, for Formula (11), A is monocyclic heterocyclyl (e.g., N-methyl piperazyl); B is bicyclic heteroaryl (e.g., 2-methyl-2H-indazolyl); L is absent; Y is N; Z is N; and R² is absent. In some embodiments, the compound of is Compound 182, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 2-methyl-2H-indazolyl); L is absent; Y is N; Z is N; and R² is absent. In some embodiments, the compound of Formulas (II), (II-a), and (II-b) is Compound 203, or a pharmaceutically acceptable salt, solvate, hydrate, tautomer, or stereoisomer thereof.

In some embodiments, for Formula (II), A is bicyclic heteroaryl (e.g., 2-methyl-2H-indazolyl); B is monocyclic heterocyclyl (e.g., piperidinyl); L is absent; Y is N; Z is N; and R² is absent. In some embodiments, the compound of Formulas (II), (II-a), and (II-b) is Compound 204, or a pharmaceutically acceptable salt, solvate, hydrate, tautomer, or stereoisomer thereof.

In some embodiments, for Formula (II), A is monocyclic heterocyclyl (e.g., piperazyl); B is bicyclic heteroaryl (e.g., 2-methyl-2H-indazolyl); L is absent; Y is N; Z is N; and R² is absent. In some embodiments, the compound of Formulas (II), (II-a), and (II-b) is Compound 225, or a pharmaceutically acceptable salt, solvate, hydrate, tautomer, or stereoisomer thereof.

In some embodiments, for Formula (II), A is monocyclic heterocyclyl (e.g., piperazyl); B is bicyclic heteroaryl (e.g., 8-fluoro-2-methylimidazo[1,2-a]pyridinyl); L is absent; Y is N; Z is N; and R² is absent. In some embodiments, the compound of Formulas (II), (II-a), and (II-b) is Compound 226, or a pharmaceutically acceptable salt, solvate, hydrate, tautomer, or stereoisomer thereof.

In some embodiments, for Formula (II), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L is —N(R³)— (e.g., —N(CH₃)—); Y is N; Z is N; and R² is absent. In some embodiments, the compound of Formulas (II), (II-a), and (II-b) is Compound 227, or a pharmaceutically acceptable salt, solvate, hydrate, tautomer, or stereoisomer thereof.

In some embodiments, for Formula (II), A is monocyclic heterocyclyl (e.g., N-methyl piperazyl); B is bicyclic heteroaryl (e.g., 8-fluoro-2-methylimidazo[1,2-a]pyridinyl); L is absent; Y is N; Z is N; and R² is absent. In some embodiments, the compound of Formulas (II), (II-a), and (II-b) is Compound 228, or a pharmaceutically acceptable salt, solvate, hydrate, tautomer, or stereoisomer thereof.

In some embodiments, for Formula (II), A is monocyclic heterocyclyl (e.g., piperazyl); B is bicyclic heteroaryl (e.g., 2-methyl-8-(trifluoromethyl)imidazo[1,2-a]pyridinyl); L is absent; Y is N; Z is N; and R² is absent. In some embodiments, the compound of Formulas (II), (II-a), and (II-b) is Compound 229, or a pharmaceutically acceptable salt, solvate, hydrate, tautomer, or stereoisomer thereof.

In some embodiments, for Formula (II), A is monocyclic heterocyclyl (e.g., N-methyl piperazyl); B is bicyclic heteroaryl (e.g., 2-methyl-8-(trifluoromethyl)imidazo[1,2-a]pyridinyl); L is absent; Y is N; Z is N; and R² is absent. In some embodiments, the compound of Formulas (II), (II-a), and (II-b) is Compound 230, or a pharmaceutically acceptable salt, solvate, hydrate, tautomer, or stereoisomer thereof.

In some embodiments, for Formula (II), A is bicyclic heteroaryl (e.g., 2,8-dimethylimidazo[1,2-b]pyridazyl); B is monocyclic heterocyclyl (e.g., piperidinyl); L is absent; Y is N; Z is N; and R² is absent. In some embodiments, the compound of Formulas (II), (II-a), and (II-b) is Compound 231, or a pharmaceutically acceptable salt, solvate, hydrate, tautomer, or stereoisomer thereof.

In some embodiments, for Formula (II), A is bicyclic heteroaryl (e.g., 8-fluoro-2-methylimidazo[1,2-a]pyridinyl); B is monocyclic heterocyclyl (e.g., piperidinyl); L is absent; Y is N; Z is N; and R² is absent. In some embodiments, the compound of Formulas (II), (II-a), and (II-b) is Compound 232, or a pharmaceutically acceptable salt, solvate, hydrate, tautomer, or stereoisomer thereof.

In some embodiments, for Formula (II), A is bicyclic heteroaryl (e.g., 2,7-dimethyl-2H-indazolyl); B is monocyclic heterocyclyl (e.g., piperidinyl); L is absent; Y is N; Z is N; and R² is absent. In some embodiments, the compound of Formulas (II), (II-a), and (II-b) is Compound 233, or a pharmaceutically acceptable salt, solvate, hydrate, tautomer, or stereoisomer thereof.

In some embodiments, for Formula (II), A is bicyclic heteroaryl (e.g., 2,7-dimethyl-2H-indazolyl); B is monocyclic heterocyclyl (e.g., N-methyl piperidinyl); L is absent; Y is N; Z is N; and R² is absent. In some embodiments, the compound of Formulas (II), (II-a), and (II-b) is Compound 234, or a pharmaceutically acceptable salt, solvate, hydrate, tautomer, or stereoisomer thereof.

In some embodiments, for Formula (II), A is bicyclic heteroaryl (e.g., 8-fluoro-2-methylimidazo[1,2-a]pyridinyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 235, or a pharmaceutically acceptable salt, solvate, hydrate, tautomer, or stereoisomer thereof.

In some embodiments, for Formula (II), A is bicyclic heteroaryl (e.g., 2,7-dimethyl-2H-indazolyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 236, or a pharmaceutically acceptable salt, solvate, hydrate, tautomer, or stereoisomer thereof.

In some embodiments, for Formula (II), A is bicyclic heteroaryl (e.g., 2,7-dimethyl-2H-indazolyl); B is monocyclic heterocyclyl (e.g., N-methyl piperidinyl); L is absent; Y is N; Z is C(R⁶) (e.g., CH); and R² is absent. In some embodiments, the compound of Formulas (II) and (II-b) is Compound 237, or a pharmaceutically acceptable salt, solvate, hydrate, tautomer, or stereoisomer thereof.

In some embodiments, for Formula (II), A is bicyclic heteroaryl (e.g., 2,8-dimethylimidazo[1,2-b]pyridazyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 238, or a pharmaceutically acceptable salt, solvate, hydrate, tautomer, or stereoisomer thereof.

In some embodiments, for Formula (II), A is bicyclic heteroaryl (e.g., 2,8-dimethylimidazo[1,2-b]pyridazyl); B is monocyclic heterocyclyl (e.g., N-methyl piperidinyl); L is absent; Y is N; Z is C(R⁶) (e.g., CH); and R² is absent. In some embodiments, the compound of Formulas (II) and (II-b) is Compound 239, or a pharmaceutically acceptable salt, solvate, hydrate, tautomer, or stereoisomer thereof.

In some embodiments, for Formula (II), A is bicyclic heteroaryl (e.g., 2,7-dimethyl-2H-pyrazolo[3,4-c]pyridinyl); B is monocyclic heterocyclyl (e.g., piperidinyl); L is absent; Y is N; Z is N; and R² is absent. In some embodiments, the compound of Formulas (II), (II-a), and (II-b) is Compound 251, or a pharmaceutically acceptable salt, solvate, hydrate, tautomer, or stereoisomer thereof.

In some embodiments, for Formula (II), A is bicyclic heteroaryl (e.g., 2-methylimidazo[1,2-a]pyrazyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 252, or a pharmaceutically acceptable salt, solvate, hydrate, tautomer, or stereoisomer thereof.

In some embodiments, for Formula (II), A is bicyclic heteroaryl (e.g., 2-methyl-6-hydroxy-2H-indazolyl); B is monocyclic heterocyclyl (e.g., piperidinyl); L is absent; Y is N; Z is N; and R² is absent. In some embodiments, the compound of Formulas (II), (II-a), and (II-b) is Compound 253, or a pharmaceutically acceptable salt, solvate, hydrate, tautomer, or stereoisomer thereof.

In some embodiments, for Formula (II), A is bicyclic heteroaryl (e.g., 2 7-fluoro-2-methyl-2H-indazolyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 257, or a pharmaceutically acceptable salt, solvate, hydrate, tautomer, or stereoisomer thereof.

In some embodiments, for Formula (II), A is bicyclic heteroaryl (e.g., 6-fluoro-2-methyl-2H-indazolyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 258, or a pharmaceutically acceptable salt, solvate, hydrate, tautomer, or stereoisomer thereof.

In some embodiments, for Formula (II), A is bicyclic heteroaryl (e.g 2-methyl-7-carbonitrile-2H-indazolyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 259, or a pharmaceutically acceptable salt, solvate, hydrate, tautomer, or stereoisomer thereof.

In some embodiments, for Formula (II), A is monocyclic heteroaryl (e.g., 3-methoxypyridazinyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 260, or a pharmaceutically acceptable salt, solvate, hydrate, tautomer, or stereoisomer thereof.

In some embodiments, for Formula (II), A is bicyclic heteroaryl (e.g., 2,8-dimethylimidazo[1,2-a]pyridinyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 261, or a pharmaceutically acceptable salt, solvate, hydrate, tautomer, or stereoisomer thereof.

In some embodiments, for Formula (II), A is bicyclic heteroaryl (e.g., 4,6-dimethylpyrazolo[1,5-a]pyrazyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 262, or a pharmaceutically acceptable salt, solvate, hydrate, tautomer, or stereoisomer thereof.

In some embodiments, for Formula (II), A is monocyclic heteroaryl (e.g., 2-methoxypyridinyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 263, or a pharmaceutically acceptable salt, solvate, hydrate, tautomer, or stereoisomer thereof.

In some embodiments, for Formula (II), A is bicyclic heteroaryl (e.g., 2-methylimidazo[1,2-a]pyrazyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 264, or a pharmaceutically acceptable salt, solvate, hydrate, tautomer, or stereoisomer thereof.

In some embodiments, for Formula (II), A is bicyclic heteroaryl (e.g., 8-fluoro-2-methylimidazo[1,2-a]pyridinyl); B is monocyclic heterocyclyl (e.g., N-methyl piperidinyl); L is absent; Y is N; Z is C(R⁶) (e.g., CH); and R² is absent. In some embodiments, the compound of Formulas (II) and (II-b) is Compound 265, or a pharmaceutically acceptable salt, solvate, hydrate, tautomer, or stereoisomer thereof.

In some embodiments, for Formula (II), A is bicyclic heteroaryl (e.g., 8-fluoro-2-methylimidazo[1,2-a]pyridinyl); B is monocyclic heterocyclyl (e.g., piperidinyl); L is absent; Y is N; Z is C(R⁶) (e.g., CH); and R² is absent. In some embodiments, the compound of Formulas (II) and (II-b) is Compound 269, or a pharmaceutically acceptable salt, solvate, hydrate, tautomer, or stereoisomer thereof.

In some embodiments, for Formula (II), A is bicyclic heteroaryl (e.g., 8-fluoro-2-methylimidazo[1,2-a]pyridinyl); B is monocyclic heterocyclyl (e.g., pyrrolidinyl); L is absent; Y is N; Z is C(R⁶) (e.g., CH); and R² is absent. In some embodiments, the compound of Formulas (II) and (II-b) is Compound 270, or a pharmaceutically acceptable salt, solvate, hydrate, tautomer, or stereoisomer thereof.

In some embodiments, for Formula (II), A is bicyclic heteroaryl (e.g., 2,7-ditnethylpyrazolo[3,4-c]pyridinyl); B is monocyclic heterocyclyl (e.g., pyrrolidinyl); L is absent; Y is N; Z is C(R⁶) (e.g., CH); and R² is absent. In some embodiments, the compound of Formulas (II) and (II-b) is Compound 271, or a pharmaceutically acceptable salt, solvate, hydrate, tautomer, or stereoisomer thereof.

In some embodiments, for Formula (II), A is bicyclic heteroaryl (e.g., 8-fluoro-2-methylimidazo[1,2-a]pyridinyl); B is monocyclic heterocyclyl (e.g., 3-fluoropiperidinyl); L is absent; Y is N; Z is C(R⁶) (e.g., CH); and R² is absent. In some embodiments, the compound of Formulas (II) and (II-b) is Compound 272, Compound 273, or a pharmaceutically acceptable salt, solvate, hydrate, tautomer, or stereoisomer thereof.

In some embodiments, for Formula (II), A is bicyclic heteroaryl (e.g., 8-fluoro-2-methylimidazo[1,2-a]pyridinyl); B is monocyclic heterocyclyl (e.g., 3,3-difluoropiperidinyl); L is absent; Y is N; Z is C(R⁶) (e.g., CH); and R² is absent. In some embodiments, the compound of Formulas (II) and (II-b) is Compound 274 or a pharmaceutically acceptable salt, solvate, hydrate, tautomer, or stereoisomer thereof.

In some embodiments, for Formula (II), A is bicyclic heteroaryl (e.g., 8-fluoro-2-methylimidazo[1,2-a]pyridinyl); B is monocyclic heterocyclyl (e.g., hexamethyleneiminyl, e.g., azepanyl); L is absent; Y is N; Z is C(R⁶) (e.g., CH); and R² is absent. In some embodiments, the compound of Formulas (II) and (II-b) is Compound 275 or a pharmaceutically acceptable salt, solvate, hydrate, tautomer, or stereoisomer thereof.

In some embodiments, for Formula (II), A is bicyclic heteroaryl (e.g., 8-fluoro-2-methylimidazo[1,2-a]pyridinyl); B is monocyclic heterocyclyl (e.g., 1,2,3,6-tetrahydropyridinyl); L is absent; Y is N; Z is C(R⁶) (e.g., CH); and R² is absent. In some embodiments, the compound of Formulas (II) and (II-b) is Compound 276 or a pharmaceutically acceptable salt, solvate, hydrate, tautomer, or stereoisomer thereof.

In some embodiments, for Formula (II), A is bicyclic heteroaryl (e.g., 8-fluoro-2-methylimidazo[1,2-a]pyridinyl); B is monocyclic heterocyclyl (e.g., 2-methylpiperidinyl); L is absent; Y is N; Z is C(R⁶) (e.g., CH); and R² is absent. In some embodiments, the compound of Formulas (II) and (II-b) is Compound 277 or a pharmaceutically acceptable salt, solvate, hydrate, tautomer, or stereoisomer thereof.

In some embodiments, for Formula (II), A is bicyclic heteroaryl (e.g., 8-fluoro-2-methylimidazo[1,2-a]pyridinyl); B is monocyclic heterocyclyl (e.g., 4-piperidonyl); L is absent; Y is N; Z is C(R⁶) (e.g., CH); and R² is absent. In some embodiments, the compound of Formulas (II) and (II-b) is Compound 279 or a pharmaceutically acceptable salt, solvate, hydrate, tautomer, or stereoisomer thereof.

In some embodiments, for Formula (II), A is bicyclic heteroaryl (e.g., 8-fluoro-2-methylimidazo[1,2-a]pyridinyl); B is monocyclic heterocyclyl (e.g., 3-hydroxypiperidinyl); L is absent; Y is N; Z is C(R⁶) (e.g., CH); and R² is absent. In some embodiments, the compound of Formulas (II) and (II-b) is Compound 280 or a pharmaceutically acceptable salt, solvate, hydrate, tautomer, or stereoisomer thereof.

In some embodiments, the compound of Formula (III) is a compound of Formula (III-a):

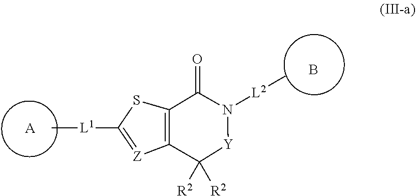

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; Y is N(R^{5a}) or C(R^{5b})(R^{5c}); Z is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently hydrogen, or C₁-C₆-alkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁵, is hydrogen, C₁-C₆-alkyl, C₁-C₆-heteroalkyl, C₁-C₆-haloalkyl; each of R^{5b} and R^{5c} is independently hydrogen, C₁-C₆-alkyl, C₁-C₆-heteroalkyl, C₁-C₆-haloalkyl, halo, or —OR^{A}; R⁶ is hydrogen, C₁-C₆-alkyl, C₁-C₆-heteroalkyl, C₁-C₆-haloalkyl, or halo; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸; each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl.

In some embodiments, A is selected from

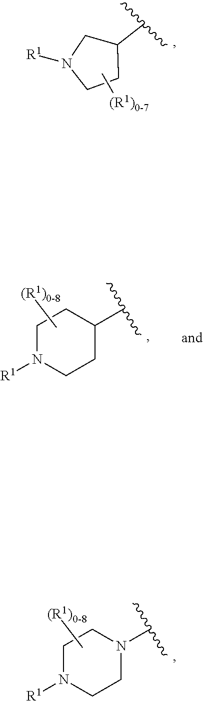

wherein R¹ is as defined herein.

In some embodiments, A is selected from

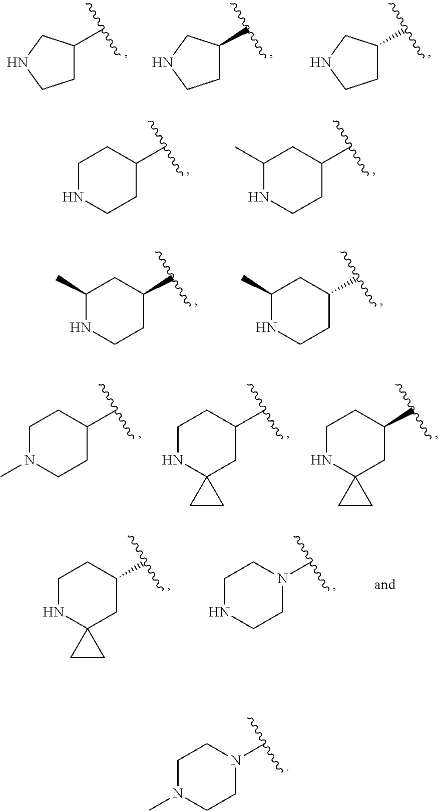

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

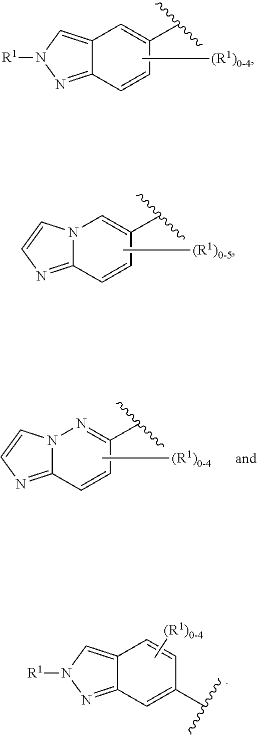

In some embodiments, A is

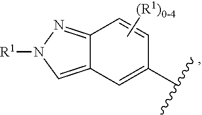

wherein R¹ is as defined herein.
In some embodiments, A is selected from

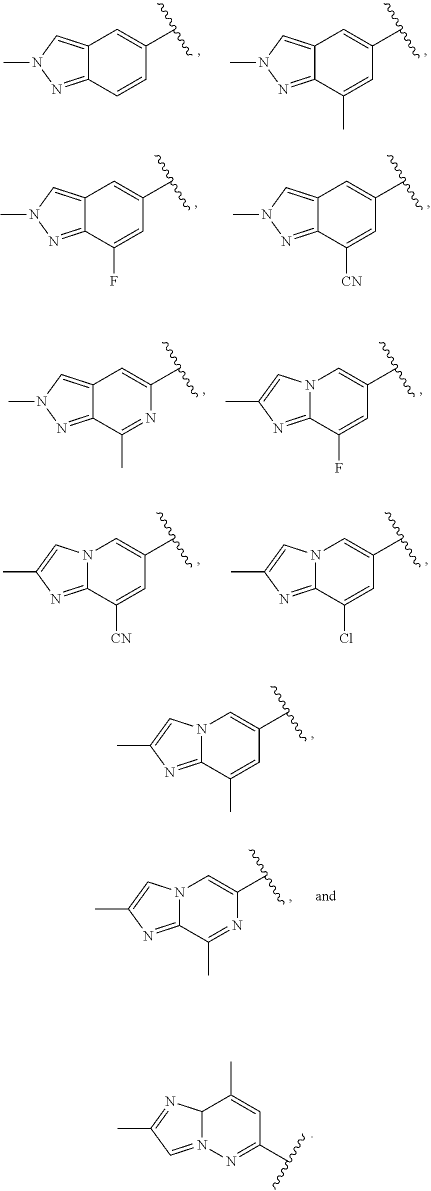

In some embodiments, A is

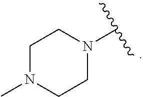 [SMILES: *N1CCN(C)CC1]

In some embodiments, A is

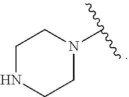 [SMILES: *N1CCNCC1]

In some embodiments, A is

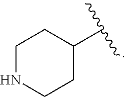 [SMILES: *C1CCNCC1]

In some embodiments, A is

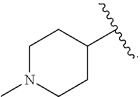 [SMILES: *C1CCN(C)CC1]

In some embodiments, A is

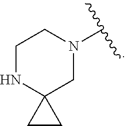 [SMILES: *N1CCNC2(CC2)C1]

In some embodiments, A is

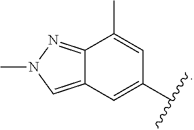 [SMILES: *c1cc(C)c2nn(C)cc2c1]

In some embodiments A is

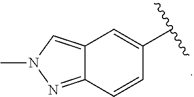 [SMILES: *c1ccc2nn(C)cc2c1]

In some embodiments, A is

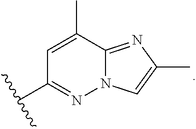 [SMILES: *c1cc(C)c2nc(C)cn2n1]

In some embodiments, A is

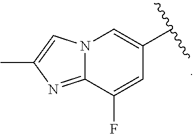 [SMILES: *c1cc(F)c2nc(C)cn2c1]

In some embodiments, B is heterocyclyl optionally substituted with one or more R¹. In some embodiments, B is monocyclic nitrogen-containing heterocyclyl. In some embodiments, B is selected from

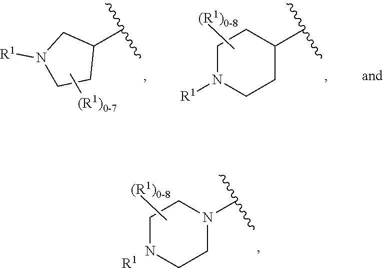

wherein R¹ is as defined herein. In some embodiments, B is selected from

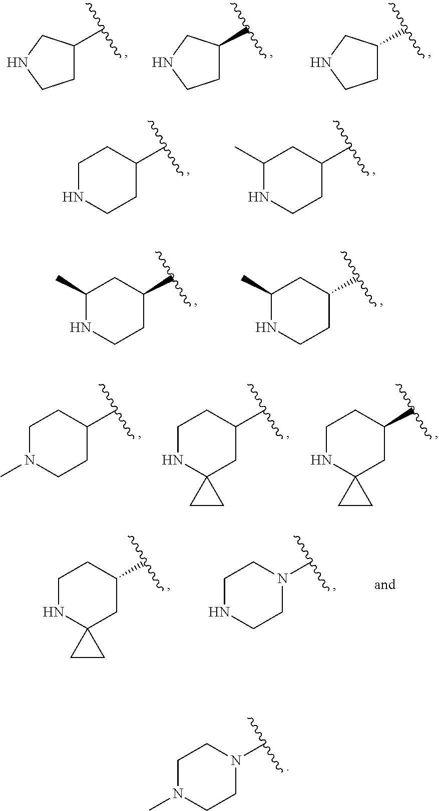

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

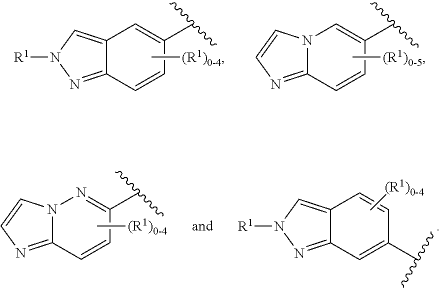

In some embodiments, A is

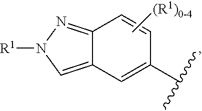

wherein R¹ is as defined herein. In some embodiments, B is selected from

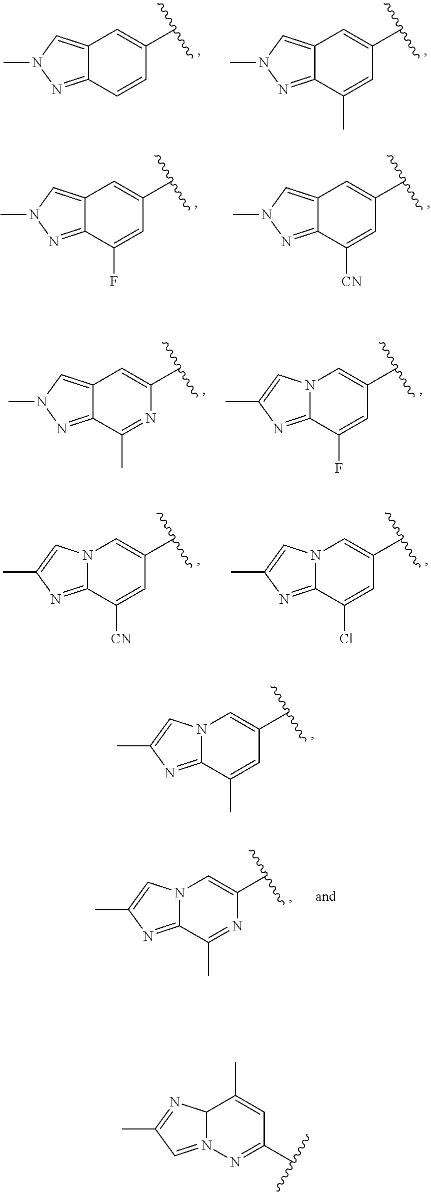

In some embodiments, B is

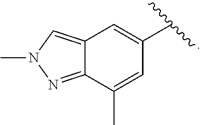 [SMILES: *c1cc(C)c2nn(C)cc2c1]

In some embodiments, B is

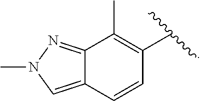 [SMILES: *c1ccc2cn(C)nc2c1C]

In some embodiments, B is

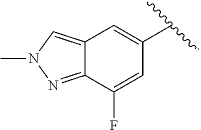 [SMILES: *c1cc(F)c2nn(C)cc2c1]

In some embodiments, B is

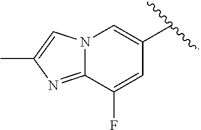 [SMILES: *c1cc(F)c2nc(C)cn2c1]

In some embodiments, B is

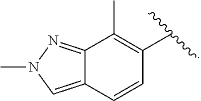 [SMILES: *c1ccc2cn(C)nc2c1C]

In some embodiments, B is

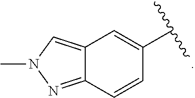 [SMILES: *c1ccc2nn(C)cc2c1]

In some embodiments, B is

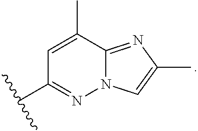 [SMILES: *c1cc(C)c2nc(C)cn2n1]

In some embodiments, B is

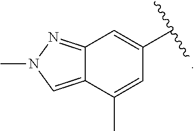 [SMILES: *c1cc(C)c2cn(C)nc2c1]

In some embodiments, B is

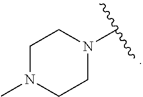 [SMILES: *N1CCN(C)CC1]

In some embodiments, B is

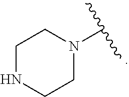 [SMILES: *N1CCNCC1]

In some embodiments, B is

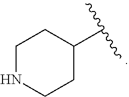 [SMILES: *C1CCNCC1]

In some embodiments, B is

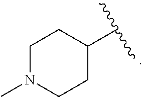 [SMILES: *C1CCN(C)CC1]

In some embodiments, B is

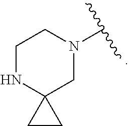 [SMILES: *N1CCNC2(CC2)C1]

In some embodiments, each of L¹ and L² is independently absent or C₁-C₆-heteroalkylene. In some embodiments, each of L¹ and L² is independently absent. In some embodiments, each of L¹ and L² is independently C₁-C₆-heteroalkylene (e.g., —N(CH₃)—). In some embodiments, L¹ and L² are absent. In some embodiments, L¹ and L² are C₁-C₆-heteroalkylene (e.g., —N(CH₃)—). In some embodiments, L² is absent or C₁-C₆-heteroalkylene (e.g., —N(CH₃)—). In some embodiments, L² is absent. In some embodiments, L² is C₁-C₆-heteroalkylene (e.g., —N(CH₃)—).

In some embodiments, Y is N(R^{5a}) or C(R^{5b}). In some embodiments, Y is N(R^{5a}) (e.g., NH). In some embodiments, Y is C(R^{5b}) (e.g., CH). In some embodiments, Y is N. In some embodiments, Y is C. In some embodiment, Y is C(R^{5b}) (e.g. CH). In some embodiments, Z is N. In some embodiments, Z is C(R⁶) (e.g., CH). In some embodiments, each R² is hydrogen.

In some embodiments, the compound of Formula (III) is a compound of Formula (III-b):

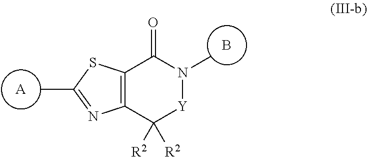

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; Y is N(R^{5a}) or C(R^{5b})(R^{5c}); Z is N or C(R⁶) each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently hydrogen, or C₁-C₆-alkyl; R^{5a} is hydrogen, C₁-C₆-alkyl, C₁-C₆-heteroalkyl, C₁-C₆-haloalkyl; each of R^{5b} and R^{5c} is independently hydrogen, C₁-C₆-alkyl, C₁-C₆-heteroalkyl, C₁-C₆-haloalkyl, halo, or —OR^{A}; R⁶ is hydrogen, C₁-C₆-alkyl, C₁-C₆-heteroalkyl, C₁-C₆-haloalkyl, or halo; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸; each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl.

In some embodiments, A is selected from

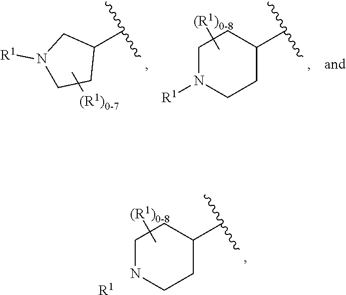

wherein R¹ is as defined herein.

In some embodiments, A is selected from

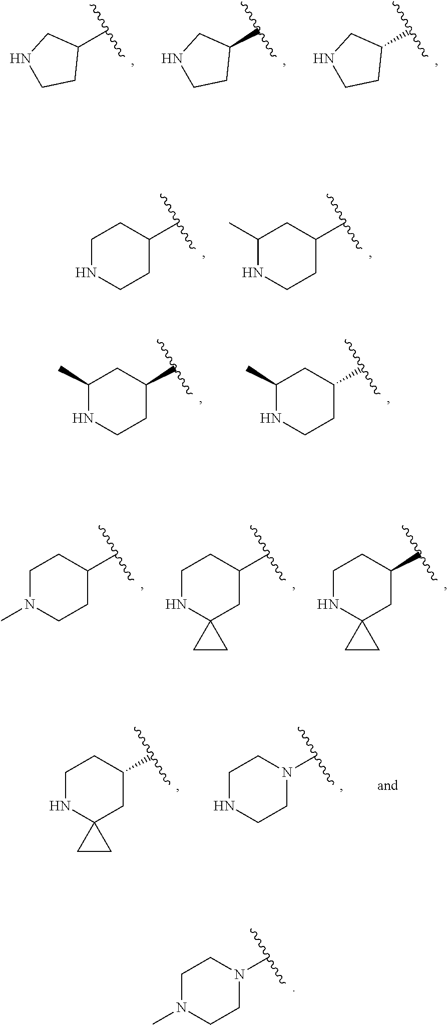

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

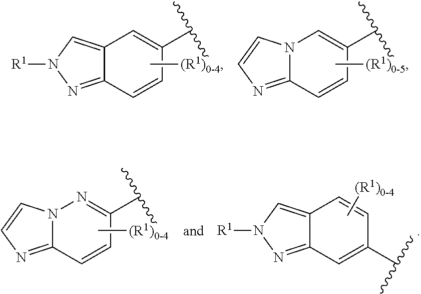

In some embodiments, A is

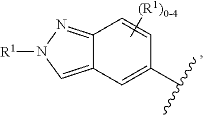

wherein R¹ is as defined herein. In some embodiments A is selected from

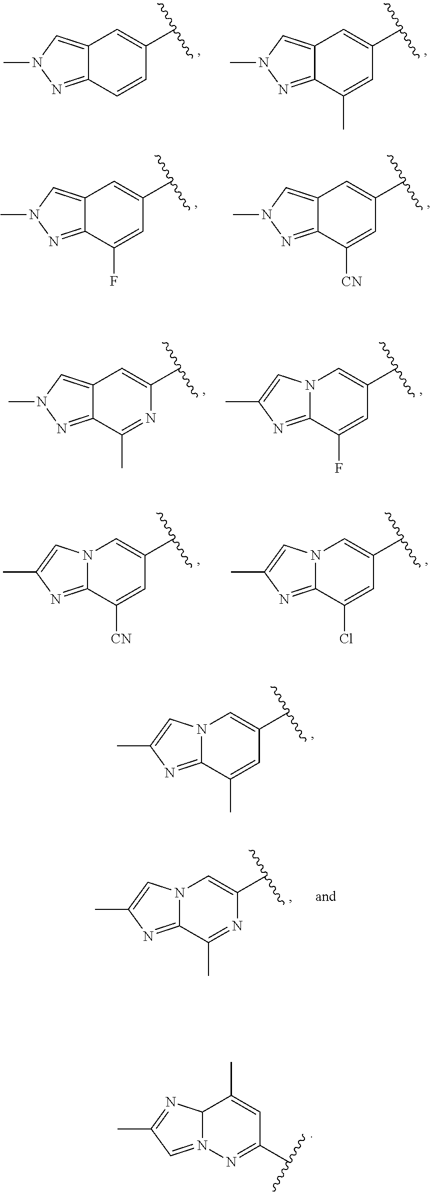

In some embodiments, A is

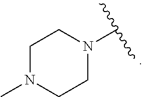 [SMILES: *N1CCN(C)CC1]

In some embodiments, A is

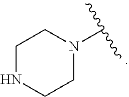 [SMILES: *N1CCNCC1]

In some embodiments, A is

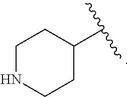 [SMILES: *C1CCNCC1]

In some embodiments, A is

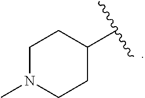 [SMILES: *C1CCN(C)CC1]

In some embodiments, A is

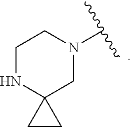 [SMILES: *N1CCNC2(CC2)C1]

In some embodiments, A is

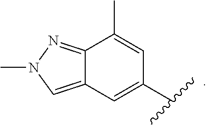 [SMILES: *c1cc(C)c2nn(C)cc2c1]

In some embodiments, A is

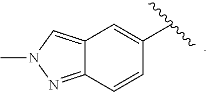 [SMILES: *c1ccc2nn(C)cc2c1]

In some embodiments, A is

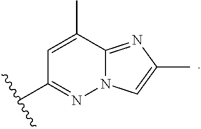 [SMILES: *c1cc(C)c2nc(C)cn2n1]

In some embodiments, A is

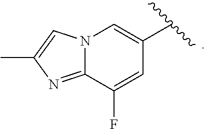 [SMILES: *c1cc(F)c2nc(C)cn2c1]

In some embodiments, B is heterocyclyl optionally substituted with one or more R¹. In some embodiments, B is monocyclic nitrogen-containing heterocyclyl. In some embodiments, B is selected from

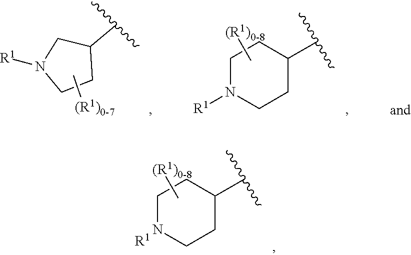

wherein R¹ is as defined herein. In some embodiments, B is selected from

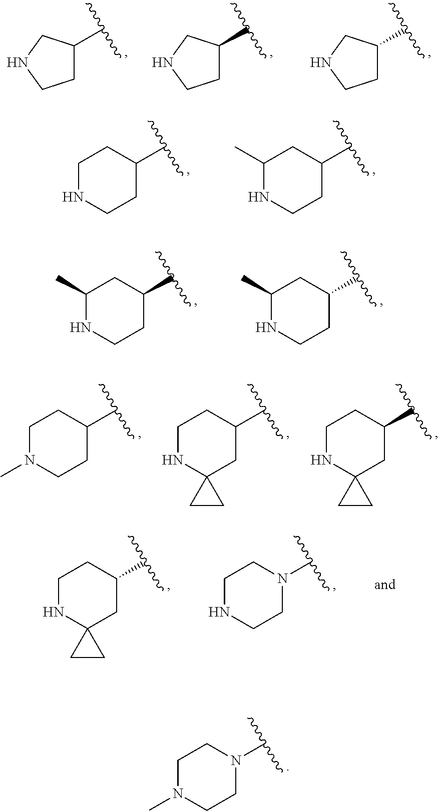

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

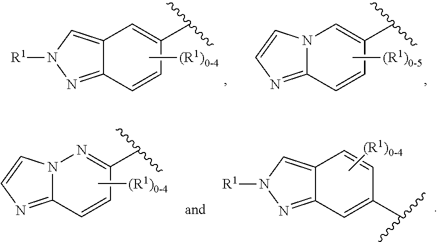

In some embodiments, A is

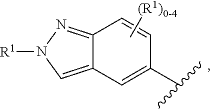

wherein R¹ is as defined herein. In some embodiments, B is selected from

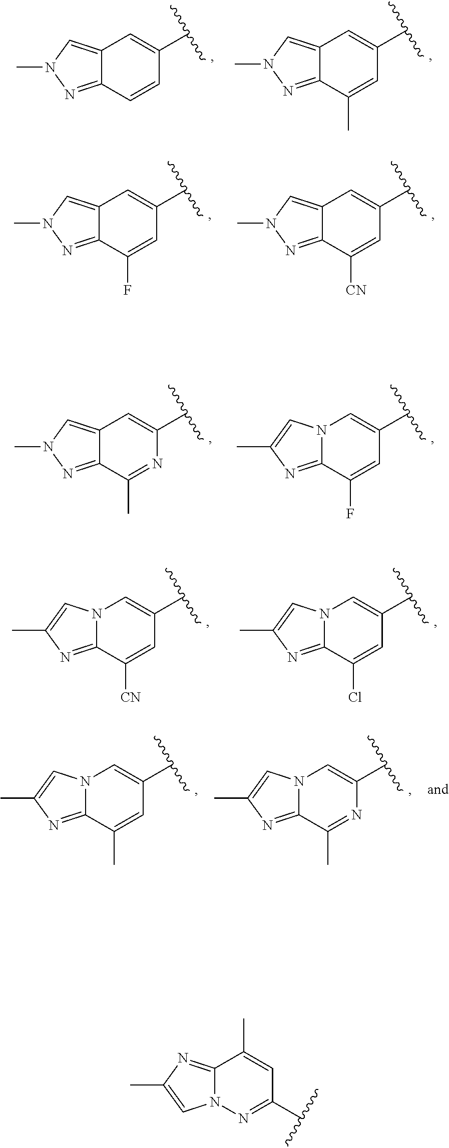

In some embodiments, B is

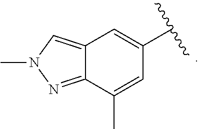 [SMILES: *c1cc(C)c2nn(C)cc2c1]

In some embodiments, B is

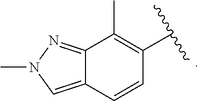 [SMILES: *c1ccc2cn(C)nc2c1C]

In some embodiments, B is

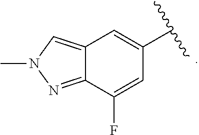 [SMILES: *c1cc(F)c2nn(C)cc2c1]

In some embodiments, B is

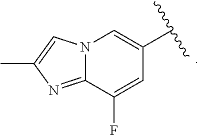 [SMILES: *c1cc(F)c2nc(C)cn2c1]

In some embodiments, B is

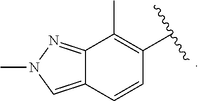 [SMILES: *c1ccc2cn(C)nc2c1C]

In some embodiments, B is

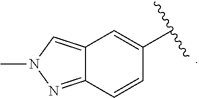 [SMILES: *c1ccc2nn(C)cc2c1]

In some embodiments, B is

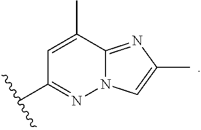 [SMILES: *c1cc(C)c2nc(C)cn2n1]

In some embodiments, B is

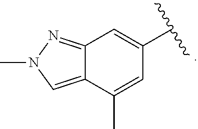 [SMILES: *c1cc(C)c2cn(C)nc2c1]

In some embodiments, B is

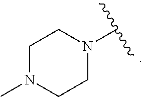 [SMILES: *N1CCN(C)CC1]

In some embodiments, B is

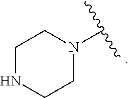 [SMILES: *N1CCNCC1]

In some embodiments, B is

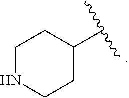 [SMILES: *C1CCNCC1]

In some embodiments, B is

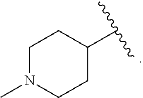 [SMILES: *C1CCN(C)CC1]

In some embodiments, B is

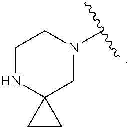 [SMILES: *N1CCNC2(CC2)C1]

In some embodiments, Y is N(R^{5a}) or C(R^{5b}). In some embodiments, Y is N(R^{5a}) (e.g., NH). In some embodiments, Y is C(R^{5b}) (e.g., CH).

In some embodiments, X is C and Y is N(R^{5a}). In some embodiments, X is C and Y is NH. In some embodiments, X is N and Y is C(R^{5b}). In some embodiments, X is N and Y is CH.

In some embodiments, Y is N. In some embodiments, Y is C. In some embodiment, Y is C(R^{5b}) (e.g. CH). In some embodiments, Z is N. In some embodiments, Z is C(R⁶) (e.g., CH).

In some embodiments, the compound of Formula (III) is a compound of Formula (III-c):

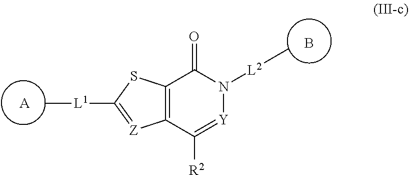

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; Y is N or C(R^{5b}); Z is N or C(R⁶) each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; R² is hydrogen or C₁-C₆-alkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R^{5b} is hydrogen, C₁-C₆-alkyl, C₁-C₆-heteroalkyl, C₁-C₆-haloalkyl, halo, or —OR^{A}; R⁶ is hydrogen, C₁-C₆-alkyl, C₁-C₆-heteroalkyl, C₁-C₆-haloalkyl, or halo; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸; each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl.

In some embodiments, A is selected from

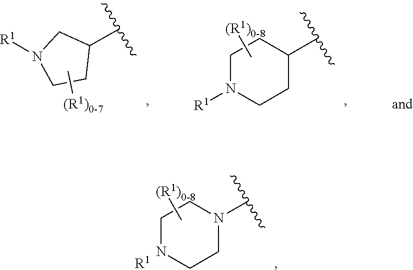

wherein R¹ is as defined herein.

In some embodiments, A is selected from

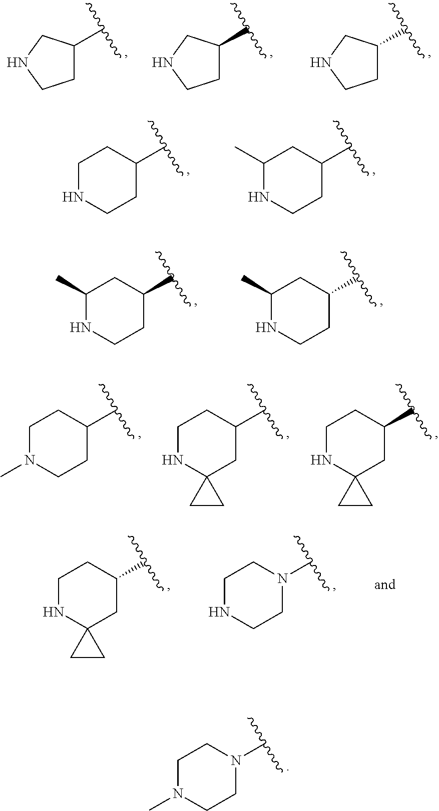

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

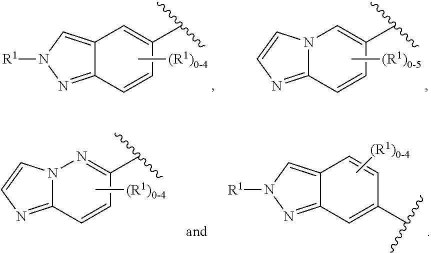

In some embodiments, A is

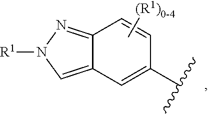

wherein R¹ is as defined herein.
In some embodiments, A is selected from

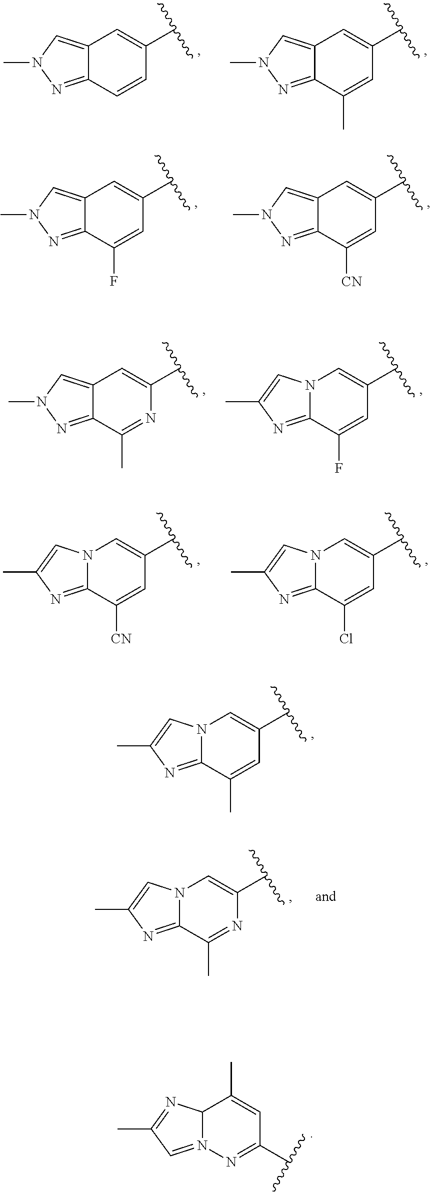

In some embodiments, A is

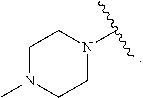 [SMILES: *N1CCN(C)CC1]

In some embodiments, A is

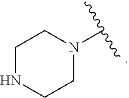 [SMILES: *N1CCNCC1]

some embodiments, A is

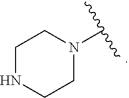 [SMILES: *N1CCNCC1]

In some embodiments A is

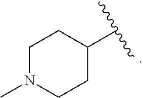 [SMILES: *C1CCN(C)CC1]

In some embodiments, A is

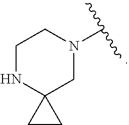 [SMILES: *N1CCNC2(CC2)C1]

In some embodiments, A is

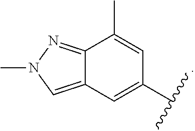 [SMILES: *c1cc(C)c2nn(C)cc2c1]

In some embodiments, A is

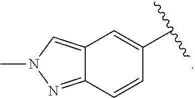 [SMILES: *c1ccc2nn(C)cc2c1]

In some embodiments, A is

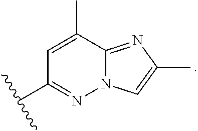 [SMILES: *c1cc(C)c2nc(C)cn2n1]

In some embodiments, A is

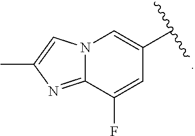 [SMILES: *c1cc(F)c2nc(C)cn2c1]

In some embodiments, B is heterocyclyl optionally substituted with one or more R¹. In some embodiments, B is monocyclic nitrogen-containing heterocyclyl. In some embodiments, B is selected from

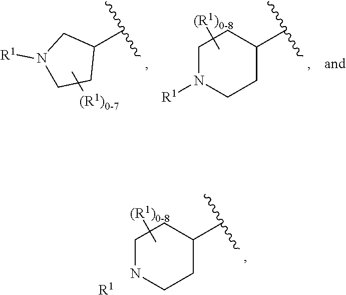

wherein R¹ is as defined herein. In some embodiments, B is selected from

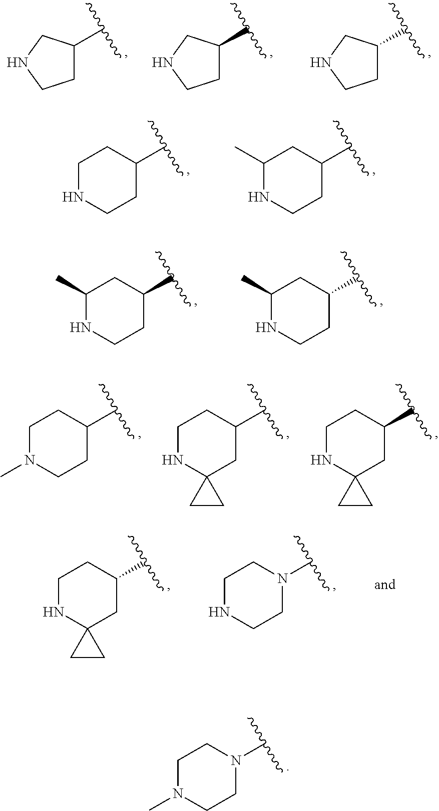

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

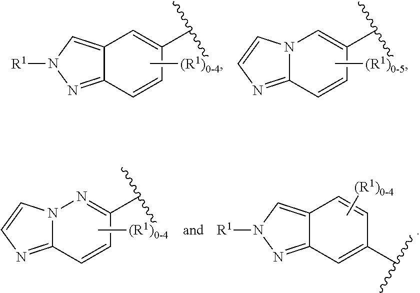

In some embodiments, A is

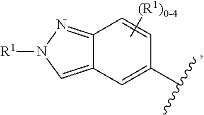

wherein R¹ is as defined herein. In some embodiments, B is selected from

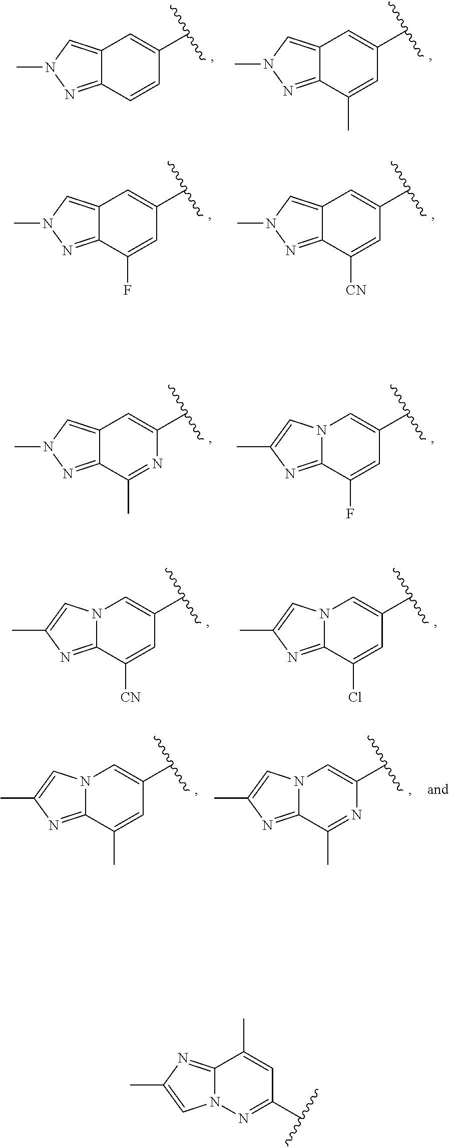

In some embodiments, B is

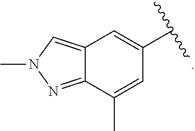 [SMILES: *c1cc(C)c2nn(C)cc2c1]

In some embodiments, B is

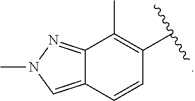 [SMILES: *c1ccc2cn(C)nc2c1C]

In some embodiments, B is

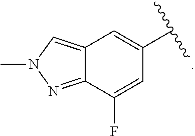 [SMILES: *c1cc(F)c2nn(C)cc2c1]

In some embodiments, B is

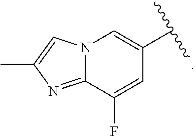 [SMILES: *c1cc(F)c2nc(C)cn2c1]

In some embodiments, B is

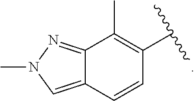 [SMILES: *c1ccc2cn(C)nc2c1C]

In some embodiments, B is

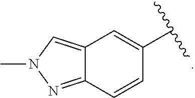 [SMILES: *c1ccc2nn(C)cc2c1]

In some embodiments, B is

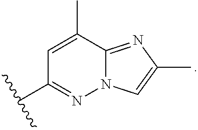 [SMILES: *c1cc(C)c2nc(C)cn2n1]

In some embodiments, B is

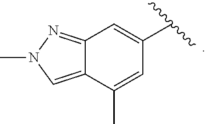 [SMILES: *c1cc(C)c2cn(C)nc2c1]

In some embodiments, B is

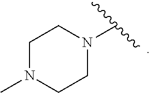 [SMILES: *N1CCN(C)CC1]

In some embodiments, B is

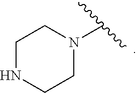 [SMILES: *N1CCNCC1]

In some embodiments, B is

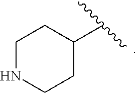 [SMILES: *C1CCNCC1]

In some embodiments, B is

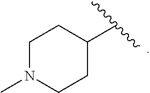 [SMILES: *C1CCN(C)CC1]

In some embodiments, B is

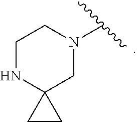 [SMILES: *N1CCNC2(CC2)C1]

In some embodiments, Y is N or C(R^{5b}). In some embodiments, Y is N. In some embodiments, Y is C(R^{5b}) (e.g., CH). In some embodiments, Z is N. In some embodiments, Z is C(R⁶) (e.g., CH). In some embodiments, R² is hydrogen.

In some embodiments, the compound of Formula (III) is a compound of Formula (III-d):

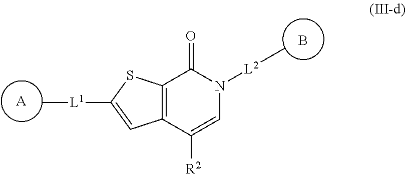

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; R² is hydrogen or C₁-C₆-alkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸; each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl.

In some embodiments, A is selected from

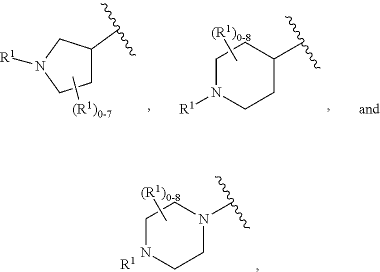

wherein R¹ is as defined herein.

In some embodiments, A is selected from

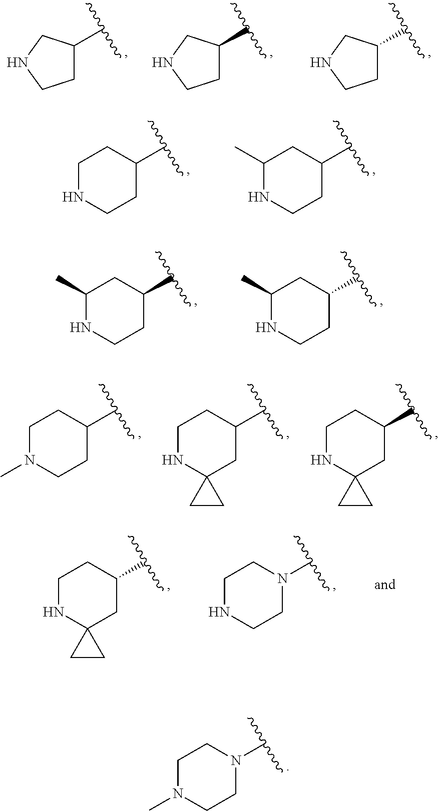

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

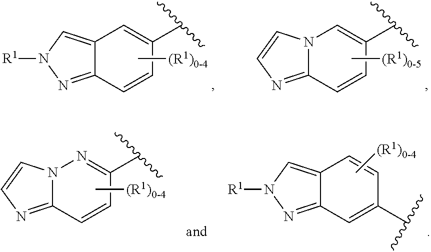

In some embodiments, A is

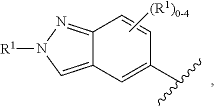

wherein R¹ is as defined herein.

In some embodiments, A is selected from

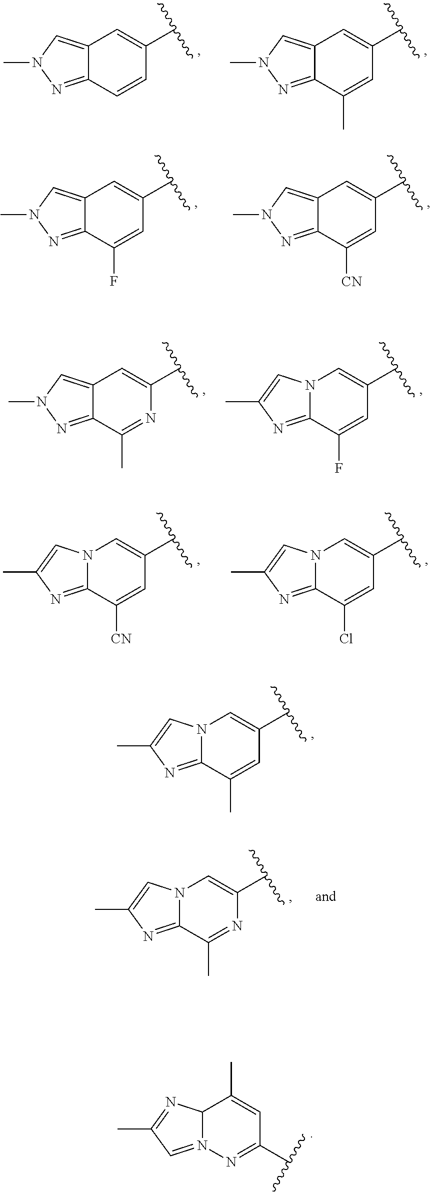

In some embodiments, A is

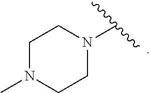 [SMILES: *N1CCN(C)CC1]

In some embodiments, A is

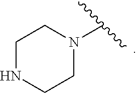 [SMILES: *N1CCNCC1]

In some embodiments, A is

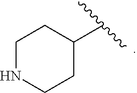 [SMILES: *C1CCNCC1]

In some embodiments, A is

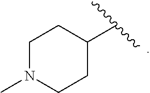 [SMILES: *C1CCN(C)CC1]

In some embodiments, A is

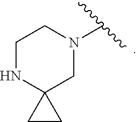 [SMILES: *N1CCNC2(CC2)C1]

In some embodiments, A is

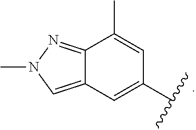 [SMILES: *c1cc(C)c2nn(C)cc2c1]

In some embodiments, A is

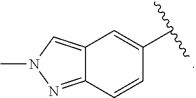 [SMILES: *c1ccc2nn(C)cc2c1]

In some embodiments,

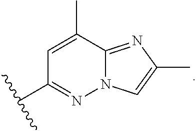 [SMILES: *c1cc(C)c2nc(C)cn2n1]

In some embodiments, A is

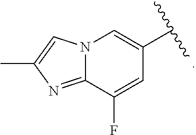 [SMILES: *c1cc(F)c2nc(C)cn2c1]

In some embodiments, B is heterocyclyl optionally substituted with one or more R¹. In some embodiments, B is monocyclic nitrogen-containing heterocyclyl. In some embodiments, B is selected from

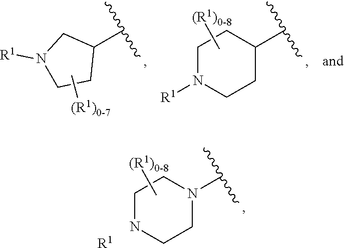

wherein R¹ is as defined herein. In some embodiments, B is selected from

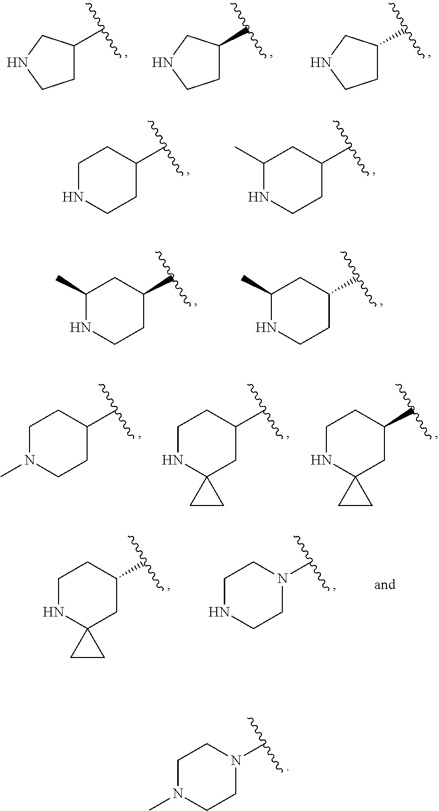

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

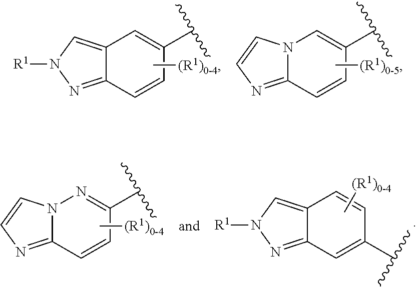

In some embodiments, A is

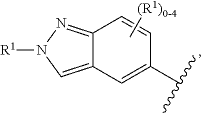

wherein R¹ is as defined herein. In some embodiments, B is selected from

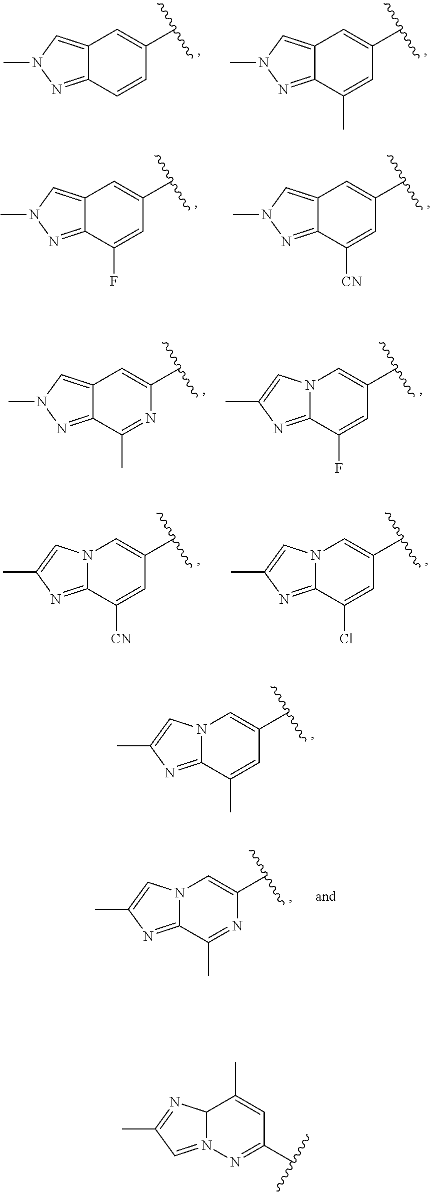

In some embodiments, B is

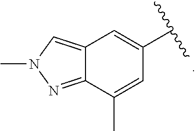 [SMILES: *c1cc(C)c2nn(C)cc2c1]

In some embodiments, B is

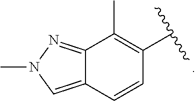 [SMILES: *c1ccc2cn(C)nc2c1C]

In some embodiments, B is

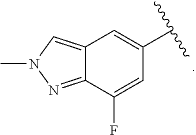 [SMILES: *c1cc(F)c2nn(C)cc2c1]

In some embodiments, B is

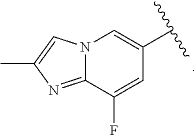 [SMILES: *c1cc(F)c2nc(C)cn2c1]

In some embodiments, B is

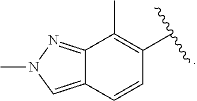 [SMILES: *c1ccc2cn(C)nc2c1C]

In some embodiments, B is

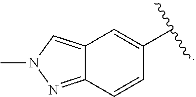 [SMILES: *c1ccc2nn(C)cc2c1]

In some embodiments B is

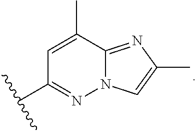 [SMILES: *c1cc(C)c2nc(C)cn2n1]

In some embodiments, B is

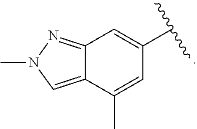 [SMILES: *c1cc(C)c2cn(C)nc2c1]

In some embodiments, B is

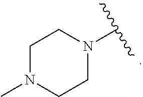 [SMILES: *N1CCN(C)CC1]

In some embodiments, B is

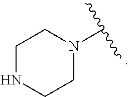 [SMILES: *N1CCNCC1]

In some embodiments, B is

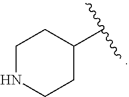 [SMILES: *C1CCNCC1]

In some embodiments, B is

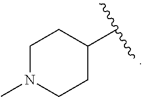 [SMILES: *C1CCN(C)CC1]

In some embodiments, B is

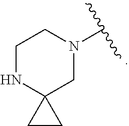 [SMILES: *N1CCNC2(CC2)C1]

In some embodiments, R² is hydrogen.

In some embodiments, the compound of Formula (III) is a compound of Formula (III-e):

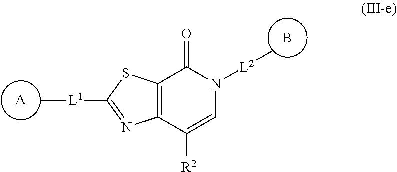

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; R² is hydrogen or C₁-C₆-alkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸; each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and Rctogether with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl.

In some embodiments, A is selected from

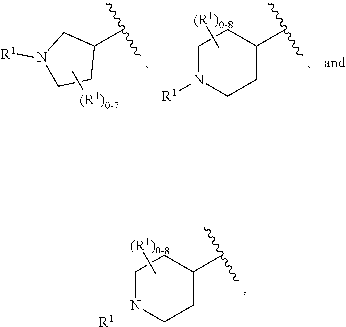

wherein R¹ is as defined herein.

In some embodiments, A is selected from

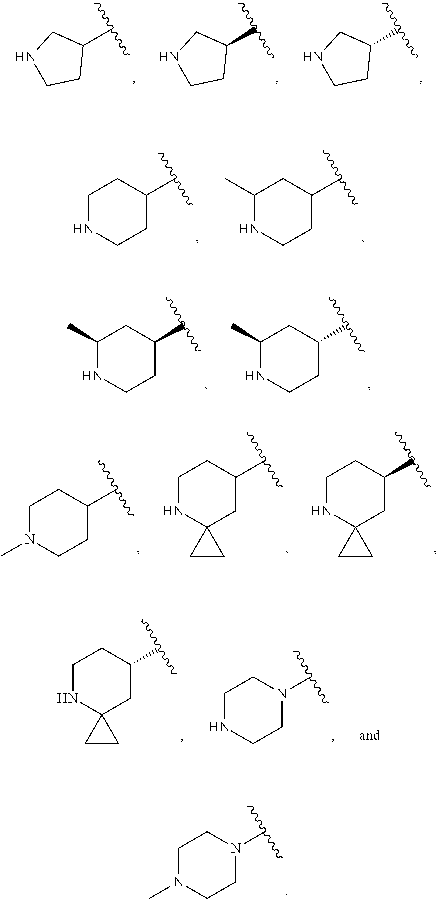

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

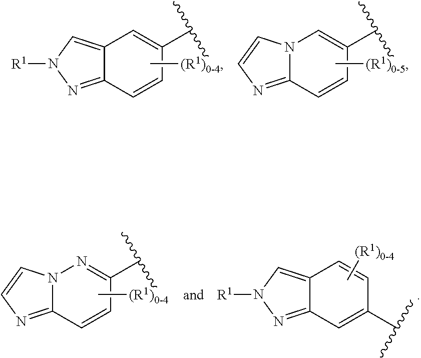

In some embodiments, A is

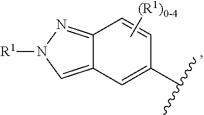

wherein R¹ is as defined herein.
In some embodiments, A is selected from

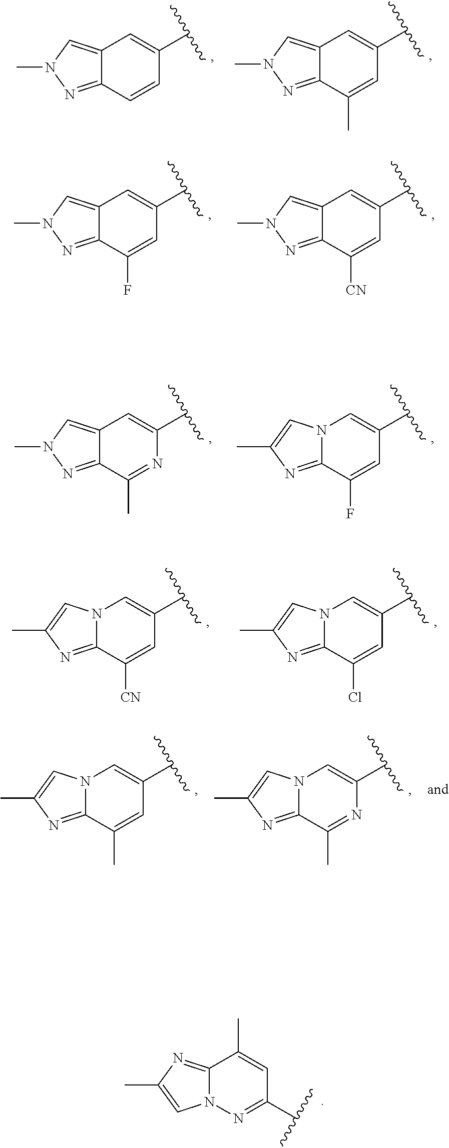

In some embodiments, A is

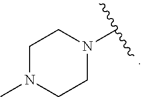 [SMILES: *N1CCN(C)CC1]

In some embodiments, A is

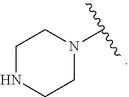 [SMILES: *N1CCNCC1]

In some embodiments A is

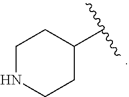 [SMILES: *C1CCNCC1]

In some embodiments A is

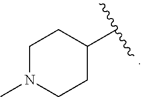 [SMILES: *C1CCN(C)CC1]

In some embodiments, A is

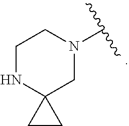 [SMILES: *N1CCNC2(CC2)C1]

In some embodiments, A is

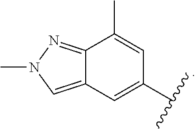 [SMILES: *c1cc(C)c2nn(C)cc2c1]

In some embodiments, A is

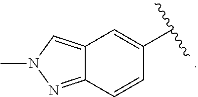 [SMILES: *c1ccc2nn(C)cc2c1]

In some embodiments A is

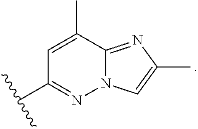 [SMILES: *c1cc(C)c2nc(C)cn2n1]

In some embodiments, A is

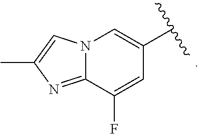 [SMILES: *c1cc(F)c2nc(C)cn2c1]

In some embodiments, B is heterocyclyl optionally substituted with one or more R¹. In some embodiments, B is monocyclic nitrogen-containing heterocyclyl. In some embodiments, B is selected from

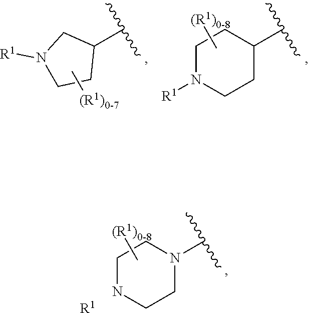

wherein R¹ is as defined herein. In some embodiments, B is selected from

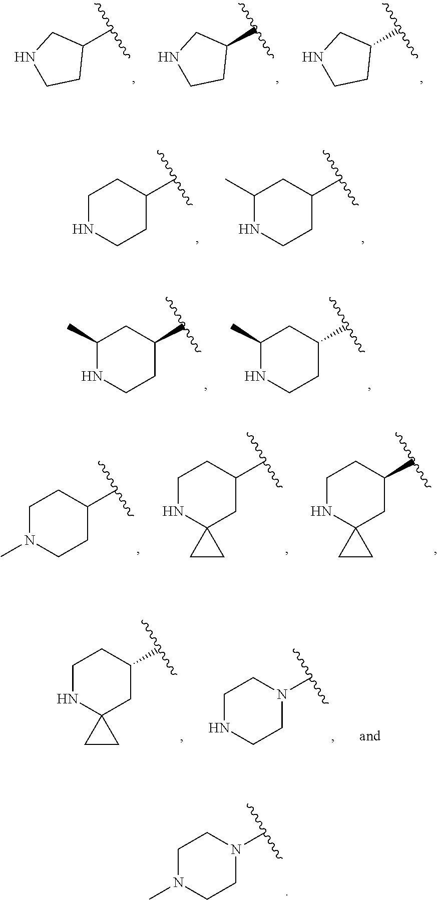

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

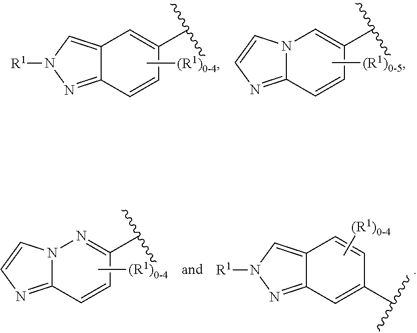

In some embodiments, A is

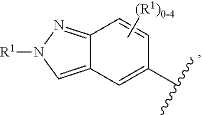

wherein R¹ is as defined herein. In some embodiments, B is selected from

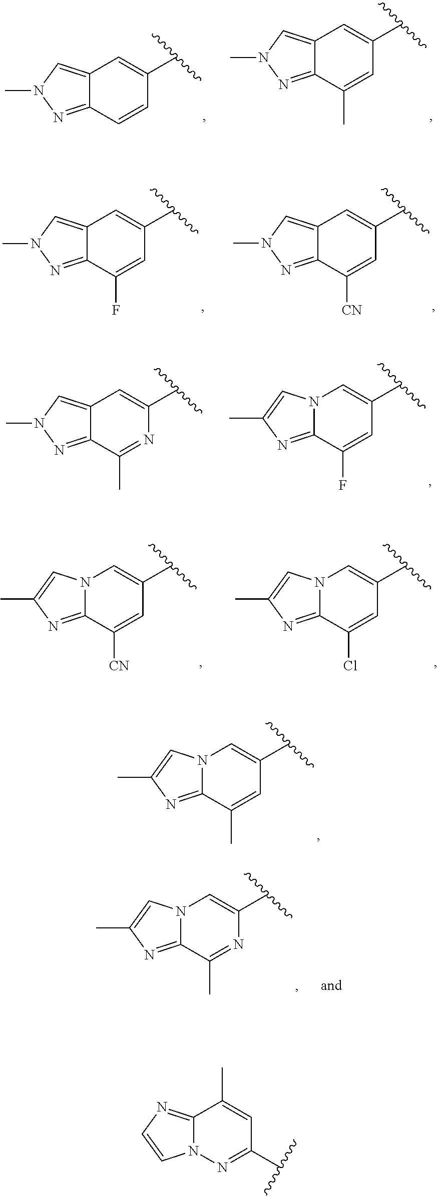

In some embodiments, B is

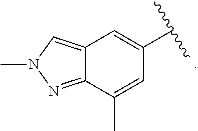 [SMILES: *c1cc(C)c2nn(C)cc2c1]

In some embodiments, B is

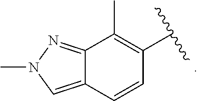 [SMILES: *c1ccc2cn(C)nc2c1C]

In some embodiments, B is

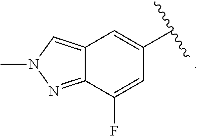 [SMILES: *c1cc(F)c2nn(C)cc2c1]

In some embodiments, B is

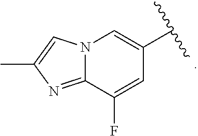 [SMILES: *c1cc(F)c2nc(C)cn2c1]

In some embodiments, B is

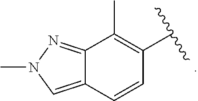 [SMILES: *c1ccc2cn(C)nc2c1C]

In some embodiments, B is

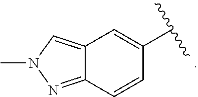 [SMILES: *c1ccc2nn(C)cc2c1]

In some embodiments, B is

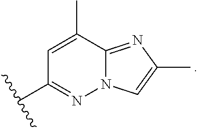 [SMILES: *c1cc(C)c2nc(C)cn2n1]

In some embodiments, B is

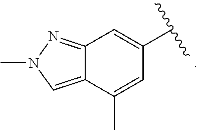 [SMILES: *c1cc(C)c2cn(C)nc2c1]

In some embodiments, B is

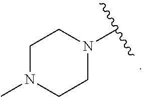 [SMILES: *N1CCN(C)CC1]

In some embodiments, B is

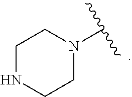 [SMILES: *N1CCNCC1]

In some embodiments, B is

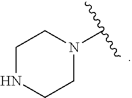 [SMILES: *N1CCNCC1]

In some embodiments, B is

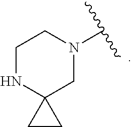 [SMILES: *N1CCNC2(CC2)C1]

In some embodiments, B is

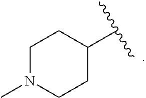 [SMILES: *C1CCN(C)CC1]

In some embodiments, R² is hydrogen.

In some embodiments, the compound of Formula (III) is a compound of Formula (III-f):

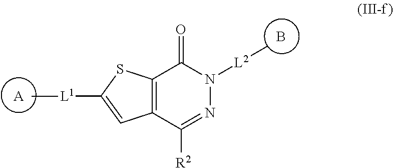

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; R² is hydrogen or C₁-C₆-alkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸; each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl.

In some embodiments, A is selected from

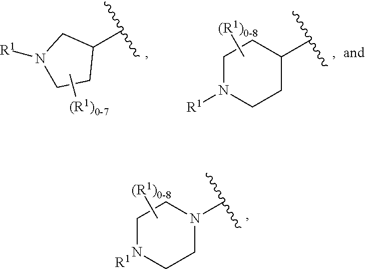

wherein R¹ is as defined herein.

In some embodiments, A is selected from

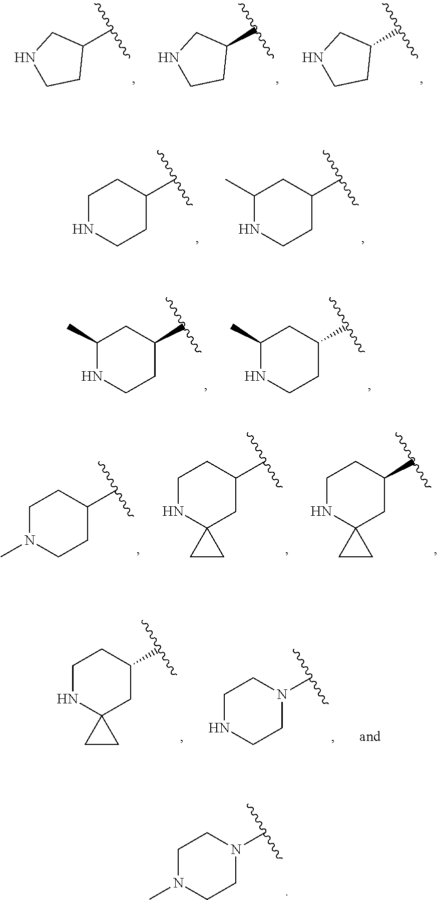

In some embodiments, A is heteroaryl optionally substituted with one or more R¹. In some embodiments, A is bicyclic nitrogen-containing heteroaryl. In some embodiments, A is optionally substituted indazolyl. In some embodiments, A is selected from

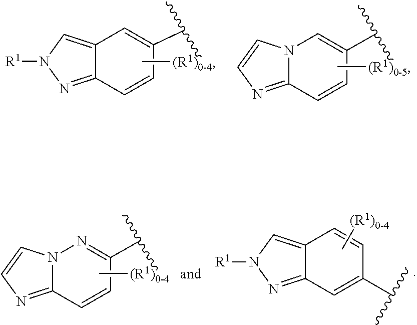

In some embodiments, A is

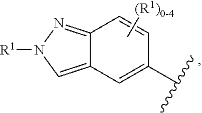

wherein R¹ is as defined herein.
In some embodiments, A is selected from

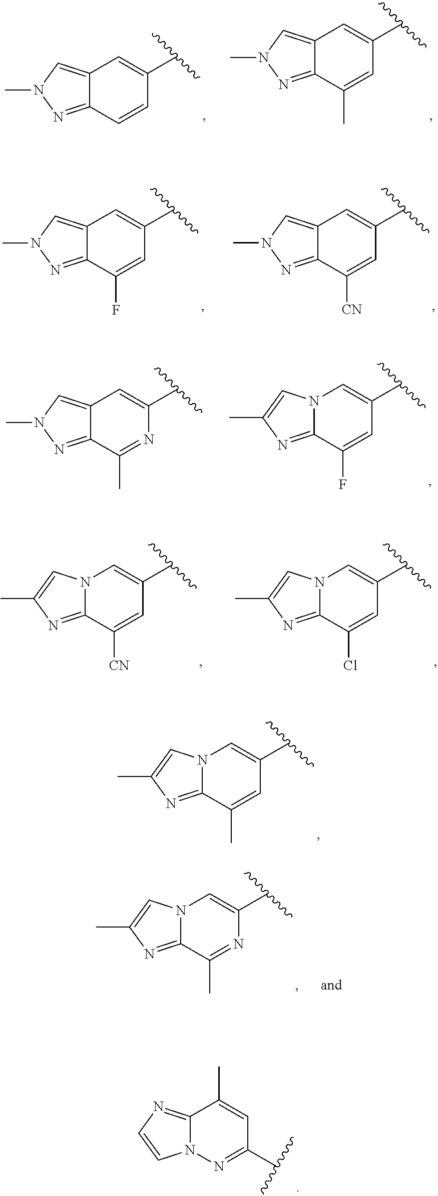

In some embodiments, A is

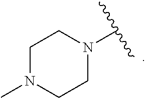 [SMILES: *N1CCN(C)CC1]

In some embodiments, A is

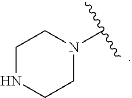 [SMILES: *N1CCNCC1]

In embodiments, A is

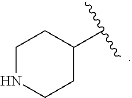 [SMILES: *C1CCNCC1]

In some embodiments A is

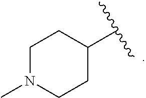 [SMILES: *C1CCN(C)CC1]

In some embodiments, A is

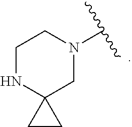 [SMILES: *N1CCNC2(CC2)C1]

In some embodiments, A is

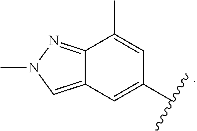 [SMILES: *c1cc(C)c2nn(C)cc2c1]

In some embodiments, A is

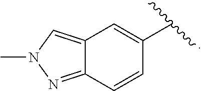 [SMILES: *c1ccc2nn(C)cc2c1]

In some embodiments, A is

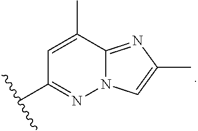 [SMILES: *c1cc(C)c2nc(C)cn2n1]

In some embodiments, A is

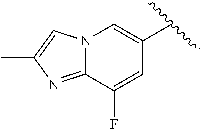 [SMILES: *c1cc(F)c2nc(C)cn2c1]

In some embodiments, B is heterocyclyl optionally substituted with one or more R¹. In some embodiments, B is monocyclic nitrogen-containing heterocyclyl. In some embodiments, B is selected from

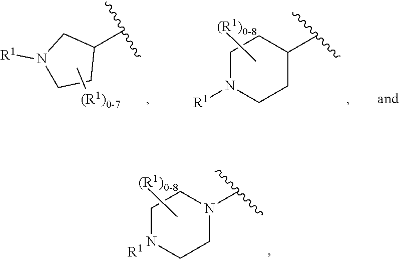

wherein R¹ is as defined herein.

In some embodiments, B is selected from

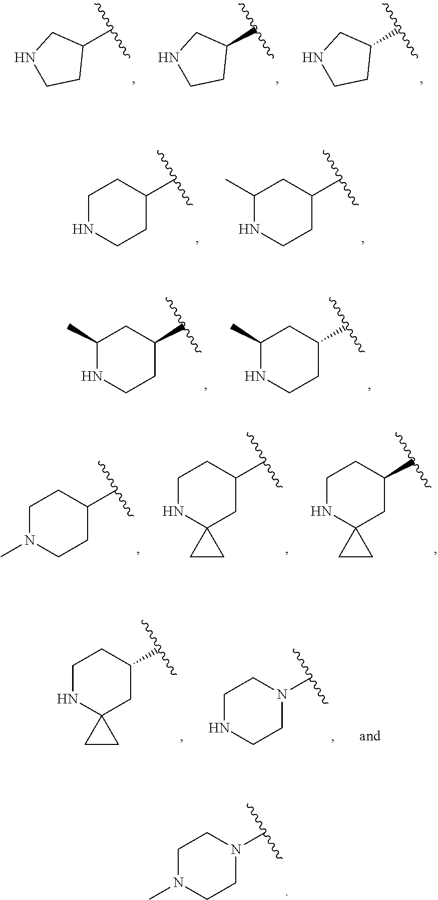

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

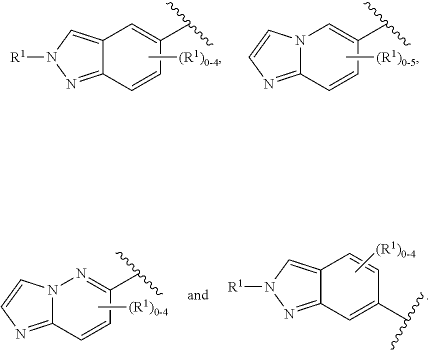

In some embodiments, A is

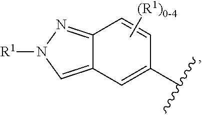

wherein R¹ is as defined herein. In some embodiments, B is selected from

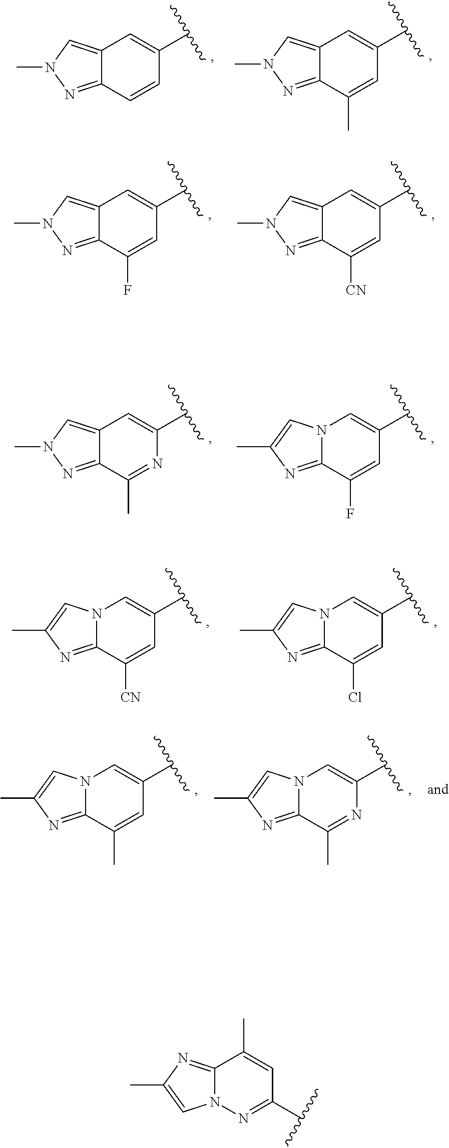

In some embodiments, B is

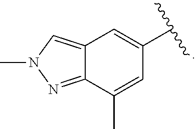 [SMILES: *c1cc(C)c2nn(C)cc2c1]

In some embodiments, B is

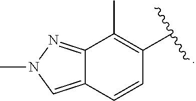 [SMILES: *c1ccc2cn(C)nc2c1C]

In some embodiments, B is

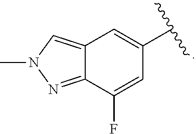 [SMILES: *c1cc(F)c2nn(C)cc2c1]

In some embodiments, B is

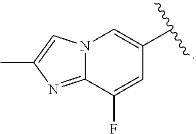 [SMILES: *c1cc(F)c2nc(C)cn2c1]

In some embodiments, B is

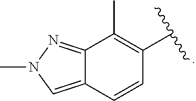 [SMILES: *c1ccc2cn(C)nc2c1C]

In some embodiments, B is

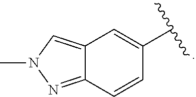 [SMILES: *c1ccc2nn(C)cc2c1]

In some embodiments, B is

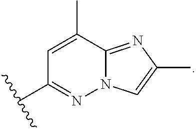 [SMILES: *c1cc(C)c2nc(C)cn2n1]

In some embodiments, B is

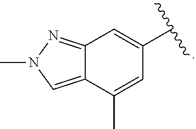 [SMILES: *c1cc(C)c2cn(C)nc2c1]

In some embodiments, B is

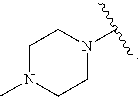 [SMILES: *N1CCN(C)CC1]

In some embodiments, B is

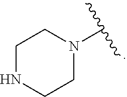 [SMILES: *N1CCNCC1]

In some embodiments, B is

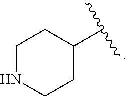 [SMILES: *C1CCNCC1]

In some embodiments, B is

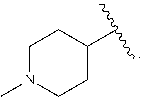 [SMILES: *C1CCN(C)CC1]

In some embodiments, B is

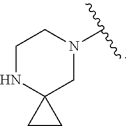 [SMILES: *N1CCNC2(CC2)C1]

In some embodiments, R² is hydrogen.

TABLE 3
Exemplary Compounds of Formula (III)

| Cmpd | |
|---|---|
| No. | Structure |
| 800 | 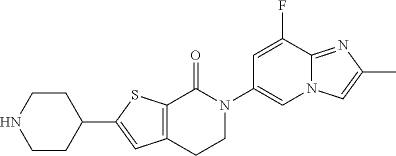 [SMILES: Cc1cn2cc(N3CCc4cc(C5CCNCC5)sc4C3=O)cc(F)c2n1] |
| 801 | 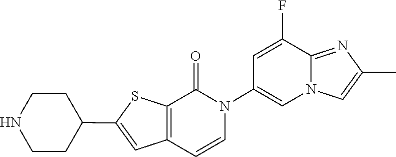 [SMILES: Cc1cn2cc(-n3ccc4cc(C5CCNCC5)sc4c3=O)cc(F)c2n1] |
| 802 | 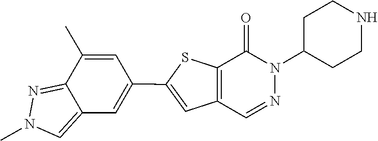 [SMILES: Cc1cc(-c2cc3cnn(C4CCNCC4)c(=O)c3s2)cc2cn(C)nc12] |
| 804 | 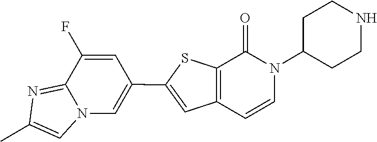 [SMILES: Cc1cn2cc(-c3cc4ccn(C5CCNCC5)c(=O)c4s3)cc(F)c2n1] |
| 805 | 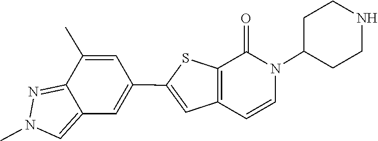 [SMILES: Cc1cc(-c2cc3ccn(C4CCNCC4)c(=O)c3s2)cc2cn(C)nc12] |
| 806 | 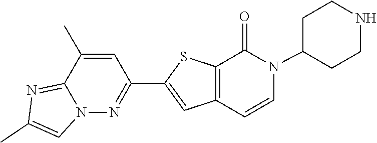 [SMILES: Cc1cn2nc(-c3cc4ccn(C5CCNCC5)c(=O)c4s3)cc(C)c2n1] |
| 807 | 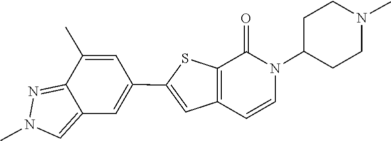 |
| 808 | 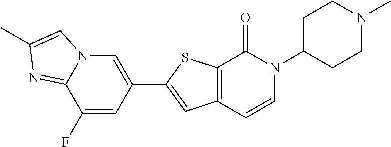 |
| 809 | 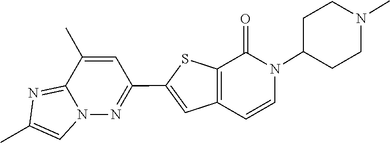 |
| 810 | 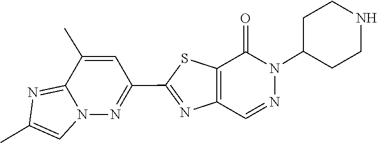 [SMILES: Cc1cn2nc(-c3nc4cnn(C5CCNCC5)c(=O)c4s3)cc(C)c2n1] |
| 811 | 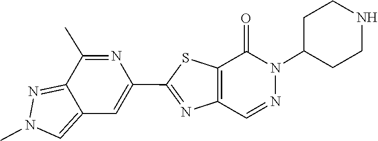 [SMILES: Cc1nc(-c2nc3cnn(C4CCNCC4)c(=O)c3s2)cc2cn(C)nc12] |
| 812 | 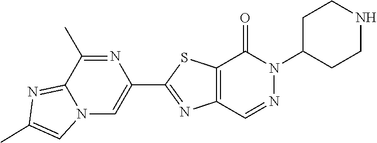 [SMILES: Cc1cn2cc(-c3nc4cnn(C5CCNCC5)c(=O)c4s3)nc(C)c2n1] |
| 824 | 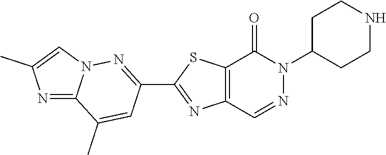 [SMILES: Cc1cn2nc(-c3nc4cnn(C5CCNCC5)c(=O)c4s3)cc(C)c2n1] |
| 825 | 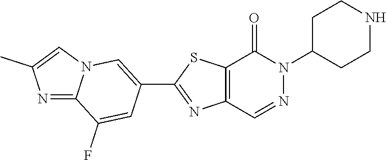 [SMILES: Cc1cn2cc(-c3nc4cnn(C5CCNCC5)c(=O)c4s3)cc(F)c2n1] |
| 826 | 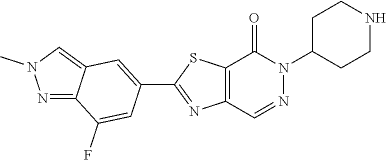 [SMILES: Cn1cc2cc(-c3nc4cnn(C5CCNCC5)c(=O)c4s3)cc(F)c2n1] |
| 827 | 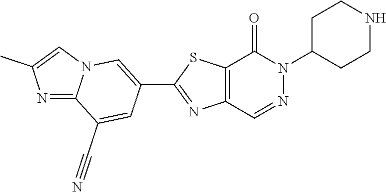 [SMILES: Cc1cn2cc(-c3nc4cnn(C5CCNCC5)c(=O)c4s3)cc(C#N)c2n1] |

In some embodiments, for Formula (III), A is monocyclic heterocyclyl (e.g., piperidinyl); B is bicyclic heteroaryl (e.g., 8-fluoro-2-methylimidazo[1,2-a]pyridinyl); L¹ and L² are each absent; Y is C(R^{4a}) (e.g., CH₂); Z is C(R^{4a}) (e.g., CH) and R² is hydrogen; and m=2. In some embodiments, the compound of Formula (III) is Compound 800, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 8-fluoro-2-methylimidazo[1,2-a]pyridinyl); L¹ and L² are each absent; Y is C(R^{4a}) (e.g., CH); Z is C(R^{4a}) (e.g., CH) and R² is hydrogen; and m=1. In some embodiments, the compound of Formula (III) is Compound 801, or a pharmaceutically acceptable salt, solvate, hydrate, tautomer, or stereoisomer thereof.

In some embodiments, for Formula (III), A is bicyclic heterocyclyl (e.g., 2,7-dimethyl-2H-indazolyl); B is monocyclic heterocyclic (e.g., piperidinyl); L¹ and L² are each absent; Y is N; Z is C(R^{4a}) (e.g., CH) and R² is hydrogen; and m=1. In some embodiments, the compound of Formula (III) is Compound 802, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., piperidinyl); L¹ and L² are each absent; Y is C(R^{4a}) (e.g., CH); Z is C(R^{4a}) (e.g., CH) and R² is hydrogen; and m=1. In some embodiments, the compound of Formula (III) is Compound 804, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., piperidinyl); L¹ and L² are each absent; Y is C(R^{4a}) (e.g., CH); Z is C(R^{4a}) (e.g., CH) and R² is hydrogen; and m=1. In some embodiments, the compound of Formula (III) is Compound 805, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazinyl); B is monocyclic heterocyclyl (e.g., piperidinyl); L¹ and L² are each absent; Y is C(R^{4a}) (e.g., CH); Z is C(R^{4a}) (e.g., CH) and R² is hydrogen; and m=1. In some embodiments, the compound of Formula (III) is Compound 806, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., N-methylpiperidinyl); L¹ and L² are each absent; Y is C(R^{4a}) (e.g., CH); Z is C(R^{4a}) (e.g., CH) and R² is hydrogen; and m=1. In some embodiments, the compound of Formula (III) is Compound 807, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., N-methylpiperidinyl); L¹ and L² are each absent; Y is C(R^{4a}) (e.g., CH); Z is C(R^{4a}) (e.g., CH) and R² is hydrogen; and m =1. In some embodiments, the compound of Formula (III) is Compound 808, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazinyl); B is monocyclic heterocyclyl (e.g., N-methylpiperidinyl); L¹ and L² are each absent; Y is C(R^{4a}) (e.g., CH); Z is C(R^{4a}) (e.g., CH) and R² is hydrogen; and m=1. In some embodiments, the compound of Formula (III) is Compound 809, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazinyl); B is monocyclic heterocyclyl (e.g., piperidinyl); L¹ and L² are each absent; Y is N; Z is N; and R² is hydrogen; and m=1. In some embodiments, the compound of Formula (III) is Compound 810, or a pharmaceutically acceptable salt, solvate, hydrate, tautomer, or stereoisomer thereof.

In some embodiments, for Formula (III), A is bicyclic heteroaryl (e.g., 2,7-dimethylpyrazolo[3,4-c]pyridinyl); B is monocyclic heterocyclyl (e.g., piperidinyl); L¹ and L² are each absent; Y is N; Z is N; and R² is hydrogen; and m=1. In some embodiments, the compound of Formula (III) is Compound 811, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-a]pyrazinyl); B is monocyclic heterocyclyl (e.g., piperidinyl); L¹ and L² are each absent; Y is N; Z is N; and R² is hydrogen; and m=1. In some embodiments, the compound of Formula (III) is Compound 812, or a pharmaceutically acceptable salt, solvate, hydrate, tautomer, or stereoisomer thereof.

### Pharmaceutical Compositions, Kits, and Administration

The present invention provides pharmaceutical compositions comprising a compound of Formula (I) or (II), e.g., a compound of Formula (I) or (II) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I) or (II) or a pharmaceutically acceptable salt thereof, and optionally a pharmaceutically acceptable excipient. In certain embodiments, the compound of Formula (I) or (II) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I) or (II) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I) or (II) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

Methods of Use Described herein are compounds useful for modulating splicing. In some embodiments, a compound of Formula (I) or (II) may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced.

In some embodiments, a compound of Formula (I) or (II) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I) or (II). In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a compound of Formula (I) or (II). In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I) or (II), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAM15, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAP10, AKAP3, AKNA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASH1L-IT1, ASNSD1, ASPM, ATAD5, ATF1, ATG4A, ATG16L2, ATM, ATN1, ATP11C, ATP6V1G3, ATP13A5, ATP7A, ATP7B, ATR, ATXN2, ATXN3, ATXN7, ATXN10, AXIN1, B2M, B4GALNT3, BBS4, BCL2, BCL2L1, BCL2-like 11 (BIM), BCL11B, BBOX1, BCS1L, BEAN1, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf30, C11orf65, C11orf70, C11orf87, C12orf51, C13orf1, C13orf15, C14orf101, C14orf118, C15orf29, C15orf42, C15orf60, C16orf33, C16orf38, C16orf48, C18orf8, C19orf42, C1orf107, C1orf114, C1orf130, C1orf149, C1orf27, C1orf71, C1orf94, CIR, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMK1D, CAMKK1, CAPN3, CAPN9, CAPSL, CARD11, CARKD, CASZ1, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDK11B, CDK13, CEBPZ, CEL, CELSR3, CENPA, CENP1, CENPT, CENTB2, CENTG2, CEP110, CEP170, CEP192, CETP, CFB, CFTR, CFH, CGN, CGNL1, CHAF1A, CHD9, CHIC2, CHL1, CHN1, CHM, CLEC16A, CLIC2, CLCN1, CLINT1, CLK1, CLPB, CLPTM1, CMIP, CMYA5, CNGA3, CNOT1, CNOT7, CNTN6, COG3, COL1A1, COL1A2, COL12A1, COL14A1, COL15A1, COL17A1, COL19A1, COL1A1, COL1A2, COL2A1, COL3A1, COL4A1, COL4A2, COL4A5, COL4A6, COL5A2, COL6A1, COL7A1, COL9A1, COL9A2, COL22A1, COL24A1, COL25A1, COL29A1, COLQ, COMTD1, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXC1, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUN1D4, DDA1, DDEF1, DDX1, DDX24, DDX4, DENND2D, DEPDC2, DES, DGAT2, DHFR, DHRS7, DHRS9, DHX8, DIP2A, DMD, DMTF1, DNAH3, DNAH8, DNAI1, DNAJA4, DNAJC13, DNAJC7, DNMT1, DNTTIP2, DOCK4, DOCK5, DOCK10, DOCK11, DOT1L, DPP3, DPP4, DPY19L2P2, DR1, DSCC1, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBN1, FBXO15, FBXO18, FBXO38, FCGBP, FECH, FEZ2, FGA, FGD6, FGFR2, FGFR1OP, FGFR1OP2, FGFR2, FGG, FGR, FIX, FKBP3, FLI1, FLJ35848, FLJ36070, FLNA, FN1, FNBP1L, FOLH1, FOSL1, FOSL2, FOXK1, FOXM1, FOXO1, FOXP4, FRAS1, FUT9, FXN, FZD3, FZD6, GAB1, GABPA, GALC, GALNT3, GAPDH, GAR1, GAS2L3, GATA3, GATAD2A, GBA, GBGT1, GCG, GCGR, GCK, GFI1, GFM1, GH1, GHR, GHV, GJA1, GLA, GLT8D1, GNAI1, GNAQ, GNAS, GNB5, GOLGB1, GOLT1A, GOLT1B, GPATCH1, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF21, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNFIA, HNFIB, HNF4A, HNF4G, HNRNPH1, HOXC10, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF1, IKZF3, ILIR2, IL5RA, IL7RA, IMMT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, ITGB3, ITGB4, ITIH1, ITPR2, IWS1, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIF15, KIF16B, KIF5A, KIF5B, KIF9, KIN, KIR2DL5B, KIR3DL2, KIR3DL3, KIT, KLF3, KLF5, KLF7, KLF10, KLF12, KLF16, KLHL20, KLK12, KLKB1, KMT2A, KMT2B, KPNA5, KRAS, KREMEN1, KRIT1, KRT5, KRTCAP2, KYNU, LICAM, L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCH1, LIMK2, LIN28B, LIN54, LMBRD1, LMBRD2, LMLN, LMNA, LMO2, LMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAGI1, MAGT1, MALT1, MAP2K1, MAP4K4, MAPK8IP3, MAPK9, MAPT, MARC1, MARCH5, MATN2, MBD3, MCF2L2, MCM6, MDGA2, MDM4, ASXL1, FUS, SPR54, MECOM, MEF2C, MEF2D, MEGF10, MEGF11, MEMO1, MET, MGA, MGAM, MGAT4A, MGAT5, MGC16169, MGC34774, MKKS, MIB1, MIER2, MITF, MKL2, MLANA, MLH1, MLL5, MLX, MME, MPDZ, MP1, MRAP2, MRPLI1, MRPL39, MRPS28, MRPS35, MS4A13, MSH2, MSH3, MSMB, MST1R, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOA1, NDC80, NDFIP2, NEB, NEDD4, NEK1, NEK5, NEK11, NF1, NF2, NFATC2, NFE2L2, NFIA, NFIB, NFIX, NFKB1, NFKB2, NFKBIL2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOL10, NOP561, NOS1, NOS2A, NOTCH1, NPAS4, NPM1, NR1D1, NR1H3, NR1H4, NR4A3, NR5A1, NRXN1, NSMAF, NSMCE2, NT5C, NT5C2, NT5C3, NUBP1, NUBPL, NUDT5, NUMA1, NUP88, NUP98, NUP160, NUPL1, OAT, OAZ1, OBFC2A, OBFC2B, OLIG2, OMA1, OPA1, OPN4, OPTN, OSBPLI1, OSBPL8, OSGEPL1, OTC, OTX2, OVOL2, OXT, PA2G4, PADI4, PAH, PAN2, PAOX, PAPOLG, PARD3, PARP1, PARVB, PAWR, PAX3, PAX8, PBGD, PBRM1, PBX2, PCBP4, PCCA, PCGF2, PCNX, PCOTH, PDCD4, PDE4D, PDE8B, PDE10A, PDIA3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTF1, PIAS1, PIEZO1, PIGF, PIGN, PIGT, PIK3C2G, PIK3CA, PIK3CD, PIK3CG, PIK3R1, PIP5KIA, PITRM1, PIWIL3, PKD1, PKHDIL1, PKD2, PKIB, PKLR, PKM1, PKM2, PLAGL2, PLCB1, PLCB4, PLCG1, PLD1, PLEKHA5, PLEKHA7, PLEKHM1, PLKR, PLXNC1, PMFBP1, POLN, POLR3D, POMT2, POSTN, POU2AF1, POU2F2, POU2F3, PPARA, PPFIA2, PPPIRI2A, PPP3CB, PPP4C, PPP4RIL, PPP4R2, PRAME, PRC1, PRDM1, PREX1, PREX2, PRIM1, PRIM2, PRKARIA, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPN11, PTPN22, PTPRD, PTPRK, PTPRM, PTPRN2, PTPRT, PUS10, PVRL2, PYGM, QRSL1, RAB11FIP2, RAB23, RAF1, RALBP1, RALGDS, RBICC1, RBL2, RBM39, RBM45, RBPJ, RBSN, REC8, RELB, RFC4, RFT1, RFTN1, RHOA, RHPN2, RIF1, RIT1, RLN3, RMND5B, RNF11, RNF32, RNFT1, RNGTT, ROCK1, ROCK2, RORA, RP1, RP6KA3, RP11-265F1, RP13-36C9, RPAP3, RPN1, RPGR, RPL22, RPL22L1, RPS6KA6, RREB1, RRM1, RRP1B, RSK2, RTEL1, RTF1, RUFY1, RUNX1, RUNX2, RXRA, RYR3, SAAL1, SAE1, SALL4, SAT1, SATB2, SBCAD, SCNIA, SCN2A, SCN3A, SCN4A, SCN5A, SCN8A, SCNA, SCN11A, SCO1, SCYL3, SDC1, SDK1, SDK2, SEC24A, SEC24D, SEC31A, SEL1L, SENP3, SENP6, SENP7, SERPINA1, SETD3, SETD4, SETDB1, SEZ6, SFRS12, SGCE, SGOL2, SGPL1, SH2DIA, SH3BGRL2, SH3PXD2A, SH3PXD2B, SH3RF2, SH3TC2, SHOC2, SIPA1L2, SIPA1L3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX, SMS, SMTN, SNCAIP, SNORD86, SNRK, SNRP70, SNX5, SNX6, SOD1, SOD10, SOS, SOS2, SOX5, SOX6, SOX8, SP1, SP2, SP3, SP110, SPAG9, SPATA13, SPATA4, SPATS1, SPECC1L, SPDEF, SP11, SPINK5, SPP2, SPTA1, SRF, SRM, SRP72, SSX3, SSX5, SSX9, STAG1, STAG2, STAMBPL1, STARD6, STAT1, STAT3, STAT5A, STAT5B, STAT6, STK17B, STX3, STXBP1, SUCLG2, SULF2, SUPT6H, SUPT16H, SV2C, SYCP2, SYT6, SYCP1, SYTL3, SYTL5, TAF2, TARDBP, TBC1D3G, TBC1D8B, TBC1D26, TBC1D29, TBCEL, TBK1, TBP, TBPL1, TBR1, TBX, TCEB3, TCF3, TCF4, TCF7L2, TCFL5, TCF12, TCP11L2, TDRD3, TEAD1, TEAD3, TEAD4, TECTB, TEK, TERF1, TERF2, TET2, TFAP2A, TFAP2B, TFAP2C, TFAP4, TFDP1, TFRC, TG, TGM7, TGS1, THAP7, THAP12, THOC2, TIAL1, TIAM2, TIMM50, TLK2, TM4SF20, TM6SF1, TMEM27, TMEM77, TMEM156, TMEM194A, TMF1, TMPRSS6, TNFRSF10A, TNFRSF10B, TNFRSF8, TNK2, TNKS, TNKS2, TOM1L1, TOM1L2, TOP2B, TP53, TP53INP1, TP53BP2, TP5313, TP63, TRAF3IP3, TRAPPC2, TRIM44, TRIM65, TRIML1, TRIML2, TRPM3, TRPM5, TRPM7, TRPS1, TSC1, TSC2, TSHB, TSPAN7, TTC17, TTF1, TTLL5, TTLL9, TTN, TTPAL, TTR, TUSC3, TXNDC10, UBE3A, UCK1, UGT1A1, UHRF1BP1, UNC45B, UNC5C, USH2A, USF2, USP1, USP6, USP18, USP38, USP39, UTP20, UTP15, UTP18, UTRN, UTX, UTY, UVRAG, UXT, VAPA, VEGFA, VPS29, VPS35, VPS39, VT11A, VT11B, VWA3B, WDFY2, WDR16, WDR17, WDR26, WDR44, WDR67, WDTC1, WRN, WRNIP1, WT1, WWC3, XBP1, XRN1, XRN2, XX—FW88277, YAP1, YARS, YBX1, YGM, YY1, ZBTB18, ZBTB20, ZC3HAV1, ZC3HC1, ZC3H7A, ZDHHC19, ZEB1, ZEB2, ZFPM1, ZFYVE1, ZFX, ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP1, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include A1CF, A4GALT, AAR2, ABAT, ABCA11P, ZNF721, ABCA5, ABHD10, ABHD13, ABHD2, ABHD6, AC000120.3, KRIT1, AC004076.1, ZNF772, AC004076.9, ZNF772, AC004223.3, RAD51D, AC004381.6, AC006486.1, ERF, AC007390.5, AC007780.1, PRKARIA, AC007998.2, INO80C, AC009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEF1, AC055811.2, FLCN, AC069368.3, ANKDD1A, AC073610.3, ARF3, AC074091.1, GPN1, AC079447.1, LIPT1, AC092587.1, AC079594.2, TRIM59, AC091060.1, C18orf21, AC092143.3, MCIR, AC093227.2, ZNF607, AC093512.2, ALDOA, AC098588.1, ANAPC10, AC107871.1, CALML4, AC114490.2, ZMYM6, AC138649.1, NIPA1, AC138894.1, CLN3, AC139768.1, AC242426.2, CHD1L, ACADM, ACAP3, ACKR2, RP11-141M3.5, KRBOX1, ACMSD, ACOT9, ACP5, ACPL2, ACSBG1, ACSF2, ACSF3, ACSL1, ACSL3, ACVR1, ADAL, ADAM29, ADAMTS10, ADAMTSL5, ADARB1, ADAT2, ADCK3, ADD3, ADGRG1, ADGRG2, ADH1B, ADIPOR1, ADNP, ADPRH, AGBL5, AGPAT1, AGPAT3, AGR2, AGTR1, AHDC1, AHI1, AHNAK, AIFM1, AIFM3, AIMP2, AK4, AKAP1, AKNAD1, CLCC1, AKR1A1, AKT1, AKT1S1, AKT2, AL139011.2, PEX19, AL157935.2, ST6GALNAC6, AL358113.1, TJP2, AL441992.2, KYAT1, AL449266.1, CLCC1, AL590556.3, LINC00339, CDC42, ALAS1, ALB, ALDH16A1, ALDH1B1, ALDH3A1, ALDH3B2, ALDOA, ALKBH2, ALPL, AMD1, AMICA1, AMN1, AMOTL2, AMY1B, AMY2B, ANAPC10, ANAPC11, ANAPC15, ANG, RNASE4, AL163636.2, ANGEL2, ANGPTL1, ANKMY1, ANKRD11, ANKRD28, ANKRD46, ANKRD9, ANKS3, ANKS3, RP11-127120.7, ANKS6, ANKZF1, ANPEP, ANXA11, ANXA2, ANXA8L2, AL603965.1, AOC3, AP000304.12, CRYZL1, AP000311.1, CRYZL1, AP000893.2, RAB30, AP001267.5, ATP5 MG, AP002495.2, AP003175.1, OR2AT4, AP003419.1, CLCF1, AP005263.1, ANKRD12, AP006621.5, AP006621.1, APIG1, AP3 M1, AP3M2, APBA2, APBB1, APLP2, APOA2, APOL1, APOL3, APTX, ARAP1, STARD10, ARF4, ARFIP1, ARFIP2, ARFRP1, ARHGAP11A, ARHGAP33, ARHGAP4, ARHGEF10, ARHGEF3, ARHGEF35, OR2A1-AS1, ARHGEF35, OR2A1-AS1, ARHGEF34P, ARID1B, ARHGEF35, OR2A20P, OR2A1-AS1, ARHGEF9, ARL1, ARL13B, ARL16, ARL6, ARMC6, ARMC8, ARMCX2, ARMCX5, RP4-769N13.6, ARMCX5-GPRASP2, BHLHB9, ARMCX5-GPRASP2, GPRASP1, ARMCX5-GPRASP2, GPRASP2, ARMCX6, ARNT2, ARPP19, ARRB2, ARSA, ART3, ASB3, GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATP1B3, ATP2C1, ATP5F1A, ATP5G2, ATP5J, ATP5MD, ATP5 PF, ATP6AP2, ATP6V0B, ATP6V1C1, ATP6V1D, ATP7B, ATXN1, ATXN1L, IST1, ATXN3, ATXN7L1, AURKA, AURKB, AXDND1, B3GALNT1, B3GALT5, AF064860.1, B3GALT5, AF064860.5, B3GNT5, B4GALT3, B4GALT4, B9D1, BACH1, BAIAP2, BANF1, BANF2, BAX, BAZ2A, BBIP1, BCHE, BCL2L14, BCL6, BCL9L, BCSIL, BDH1, BDKRB2, AL355102.2, BEST1, BEST3, BEX4, BHLHB9, BID, BIN3, BIRC2, BIVM, BIVM-ERCC5, BIVM, BLCAP, BLK, BLOC1S1, RP11-644F5.10, BLOC1S6, AC090527.2, BLOC1S6, RP11-96020.4, BLVRA, BMF, BOLA1, BORCS8-MEF2B, BORCS8, BRCA1, BRD1, BRDT, BRINP3, BROX, BTBD10, BTBD3, BTBD9, BTD, BTF3L4, BTNL9, BUB1B-PAK6, PAK6, BUB3, C10orf68, C11orf1, C11orf48, C11orf54, C11orf54, AP001273.2, C11orf57, C11orf63, C11orf82, C12orf23, C12orf4, C12orf65, C12orf79, C14orf159, C14orf93, C17orf62, C18orf21, C19orf12, C19orf40, C19orf47, C19orf48, C19orf54, CID, C1GALT1, C1QB, C1QTNF1, C1S, C1orf101, C1orf112, C1orf116, C1orf159, C1orf63, C2, C2, CFB, C20orf27, C21orf58, C2CD4D, C2orf15, LIPT1, MRPL30, C2orf80, C2orf81, C3orf14, C3orf17, C3orf18, C3orf22, C3orf33, AC104472.3, C4orf33, C5orf28, C5orf34, C6orf118, C6orf203, C6orf211, C6orf48, C7orf50, C7orf55, C7orf55-LUC7L2, LUC7L2, C8orf44-SGK3, C8orf44, C8orf59, C9, DAB2, C9orf153, C9orf9, CA5BP1, CA5B, CABYR, CALCA, CALCOCO1, CALCOCO2, CALM1, CALM3, CALML4, RP11-315D16.2, CALN1, CALU, CANT1, CANX, CAP1, CAPN12, CAPS2, CARD8, CARHSP1, CARNS1, CASC1, CASP3, CASP7, CBFA2T2, CBS, CBY1, CCBL1, CCBL2, RBMXL1, CCDC12, CCDC126, CCDC14, CCDC149, CCDC150, CCDC169-SOHLH2, CCDC169, CCDC171, CCDC37, CCDC41, CCDC57, CCDC63, CCDC7, CCDC74B, CCDC77, CCDC82, CCDC90B, CCDC91, CCDC92, CCNE1, CCHCR1, CCL28, CONB11P1, CCNC, CCND3, CCNG1, CCP110, CCR9, CCT7, CCT8, CD151, CD1D, CD200, CD22, CD226, CD276, CD36, CD59, CDC26, CDC42, CDC42SE1, CDC42SE2, CDHR3, CDK10, CDK16, CDK4, CDKAL1, CDKL3, CTD-2410N18.4, CDKN1A, CDKN2A, CDNF, CEBPZOS, CELF1, CEMIP, CENPK, CEP170B, CEP250, CEP57, CEP57L1, CEP63, CERS4, CFL1, CFL2, CFLAR, CGNL1, CHCHD7, CHD1L, CHD8, CHER, ZNF605, CHIA, CHID1, CHL1, CHM, CHMP1A, CHMP3, RNF103-CHMP3, CHRNA2, CIDEC, CIRBP, CITED1, CKLF-CMTM1, CMTM1, CKMT1B, CLDN12, CTB-13L3.1, CLDND1, AC021660.3, CLDND1, CPOX, CLHC1, CLIP1, CLUL1, CMC4, MTCP1, CNDP2, CNEN, CNOT1, CNOT6, CNOT7, CNOT8, CNR1, CNR2, CNTFR, CNTRL, COA1, COASY, COCH, COL8A1, COLCA1, COLEC11, COMMD3-BMI1, BMI1, COPS5, COPS7B, COQ8A, CORO6, COTL1, COX14, RP4-60503.4, COX7A2, COX7A2L, COX7B2, CPA4, CPA5, CPEB1, CPNE1, AL109827.1, RBM12, CPNE1, RP1-309K20.6, RBM12, CPNE3, CPSF3L, CPT1C, CREB3L2, CREM, CRP, CRYZ, CS,AC073896.1, CS, RP11-977G19.10, CSAD, CSDE1, CSF2RA, CSGALNACT1, CSK, CSNK2A1, CSRNP2, CT45A4, CT45A4, CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNBIP1, CTNND1, CTPS2, CTSB, CTSL, CTTN, CUL2, CUL9, CWC15, CXorf40B, CYB561A3, CYBC1, CYLD, CYP11A1, CYP2R1, CYP4B1, CYP4F22, DAG1, DAGLB, KDELR2, DARS, DBNL, DCAF11, DCAF8, PEX19, DCLRE1C, DCTD, DCTN1, DCTN4, DCUN1D2, DDR1, DDX11, DDX19B, AC012184.2, DDX19B, RP11-529K1.3, DDX25, DDX39B, ATP6VIG2-DDX39B, SNORD84, DDX42, DDX60L, DEDD, DEDD2, DEFA1, DEFA1B, DEFA1B, DEFA3, DENND1C, DENND2A, DENND4B, DET1, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKN, DMTF1, DMTN, DNAJC14, DNAJC19, DNAL1, DNASEIL1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA1, DUS2, DUSP10, DUSP13, DUSP18, DUSP22, DYDC1, DYDC2, DYNLL1, DYNLT1, DYRK1A, DYRK2, DYRK4, RP11-500M8.7, DZIP1L, E2F6, ECHDC1, ECSIT, ECT2, EDC3, EDEM1, EDEM2, MMP24-AS1, RP4-61404.11, EEF1AKNMT, EEF1D, EFEMP1, EFHC1, EGFL7, EHF, EI24, EIF1AD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOVL1, ELOVL7, ELP1, ELP6, EML3, EMP3, ENC1, ENDOV, ENO1, ENPP5, ENTHD2, ENTPD6, EP400NL, EPB41L1, EPDR1, NME8, EPHX1, EPM2A, EPN1, EPN2, EPN3, EPS8L2, ERBB3, ERC1, ERCC1, ERG, ERI2, ERI2, DCUN1D3, ERLIN2, ERMARD, ERRFI1, ESR2, RP11-544120.2, ESRRA, ESRRB, ESRRG, ETFA, ETFRF1, ETV1, ETV4, ETV7, EVA1A, EVC2, EVX1, EXD2, EXO5, EXOC1, EXOC2, FAAP24, FABP6, FADS1, FADS2, FAHD2B, FAM107B, FAM111A, FAM111B, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANC1, FANK1, FAR2, FAXC, FAXDC2, FBF1, FBH1, FBXL4, FBXO18, FBXO22, FBXO31, FBXO41, FBXO44, FBXO45, FBXW9, FCHO1, FCHSD2, FDFT1, FDPS, FER, FETUB, FGD4, FGF1, FGFR1, FGFRL1, FGL1, FHL2, FIBCD1, FIGNL1, FIGNL1, DDC, FKBP5, FKRP, FLRT2, FLRT3, FMC1, LUC7L2, FMC1-LUC7L2, FNDC3B, FOLH1, FOLR1, FOXP1, FOXK1, FOXM1, FOXO1, FOXP4, AC097634.4, FOXRED1, FPR1, FPR2, FRG1B, FRS2, FTO, FTSJ1, FUK, FUT10, FUT3, FUT6, FXYD3, FZD3, G2E3, GAA, GABARAPL1, GABPB1, GABRA5, GAL3ST1, GALE, GALNT11, GALNT14, GALNT6, GAPVD1, GARNL3, GAS2L3, GAS8, GATA1, GATA2, GATA4, GBA, GCNT1, GDPD2, GDPD5, GEMIN7, MARK4, GEMIN8, GGA3, GGACT, AL356966.1, GGPS1, GHRL, GID8, GIGYF2, GIMAP8, GIPC1, GJB1, GJB6, GLB1L, GLI1, GLT8D1, GMFG, GMPR2, GNAI2, GNAQ, GNB1, GNB2, GNE, GNG2, GNGT2, GNPDA1, GNPDA2, GOLGA3, CHFR, GOLGA4, GOLPH3L, GOLT1B, GPBP1L1, GPER1, GPR116, GPR141, EPDR1, GPR155, GPR161, GPR56, GPR63, GPR75-ASB3, ASB3, GPR85, GPSM2, GRAMD1B, GRB10, GRB7, GREM2, GRIA2, GSDMB, GSE1, GSN, GSTA4, GSTZ1, GTDC1, GTF2H1, GTF2H4, VARS2, GTF3C2, GUCY1A3, GUCY1B3, GUK1, GULP1, GYPC, GYS1, GZF1, HAGH, HAO2, HAPLN3, HAVCR1, HAX1, HBG2, AC104389.4, HBG2, AC104389.4, HBE1, HBG2, AC104389.4, HBE1, OR51B5, HBG2, HBE1, AC104389.28, HBS1L, HCFC1R1, HCK, HDAC2, HDAC6, HDAC7, HDLBP, HEATR4, HECTD4, HEXIM2, HHAT, HHATL, CCDC13, HINFP, HIRA, C22orf39, HIVEP3, HJV, HKR1, HLF, HMBOX1, HMGA1, HMGB3, HMGCR, HMGN4, HMOX2, HNRNPC, HNRNPD, HNRNPH1, HNRNPH3, HNRNPR, HOMER3, HOPX, HOXA3, HOXB3, HOXB3, HOXB4, HOXC4, HOXD3, HOXD3, HOXD4, HPCAL1, HPS4, HPS5, HRH1, HS3ST3A1, HSH2D, HSP90AA1, HSPD1, HTT, HUWE1, HYOU1, IAH1, ICAIL, ICAM2, ICE2, ICK, IDH2, IDH3G, IDS, IF127, IF144, IFT20, IFT22, IFT88, IGF2, INS-IGF2, IGF2BP3, IGFBP6, IKBKAP, IKBKB, IL11, IL18BP, IL18RAP, IL1RAP, IL1RL1, IL18R1, IL1RN, IL32, IL411, NUP62, AC011452.1, IL411, NUP62, CTC-326K19.6, IL6ST, ILVBL, IMMP1L, IMPDH1, INCA1, ING1, INIP, INPP1, INPP5J, INPP5K, INSIG2, INTS11, INTS12, INTS14, IP6K2, IP6K3, IPO11, LRRC70, IQCE, IQGAP3, IRAK4, IRF3, IRF5, IRF6, ISG20, IST1, ISYNA1, ITFG2, ITGBIBP1, ITGB7, ITIH4, RP5-966M1.6, ITPRIPL1, JADE1, JAK2, JARID2, JDP2, KANK1, KANK1, RP11-31F19.1, KANK2, KANSL1L, KAT6A, KBTBD2, KBTBD3, KCNAB2, KCNE3, KCNG1, KCNJ16, KCNJ9, KCNMB2, AC117457.1, LINC01014, KCTD20, KCTD7, RABGEF1, KDM1B, KDM4A, AL451062.3, KHNYN, KIAA0040, KIAA0125, KIAA0196, KIAA0226L, PPP1R2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRREL1, KITLG, KLC1, APOPT1, AL139300.1, KLC4, KLHDC4, KLHDC8A, KLHL13, KLHL18, KLHL2, KLHL24, KLHL7, KLK11, KLK2, KLK5, KLK6, KLK7, KNOP1, KRBA2, AC135178.2, KRBA2, RP11-849F2.7, KRIT1, KRT15, KRT8, KTN1, KXD1, KYAT3, RBMXL1, KYNU, L3MBTL1, LACC1, LARGE, LARP4, LARP7, LAT2, LBHD1, LCA5, LCA5L, LCTL, LEPROTL1, LGALS8, LGALS9C, LGMN, LHFPL2, LIG4, LIMCH1, LIMK2, LIMS2, LINC00921, ZNF263, LIPF, LLGL2, LMAN2L, LMCD1, LMF1, RP11-161M6.2, LMO1, LMO3, LOXHD1, LPAR1, LPAR2, LPAR4, LPAR5, LPAR6, LPHN1, LPIN2, LPIN3, LPP, LRFN5, LRIF1, LRMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMC1-LUC7L2, LUC7L3, LUZP1, LYG1, LYL1, LYPD4, LYPD6B, LYRM1, LYRM5, LYSMD4, MACC1, MADIL1, MADIL1, AC069288.1, MAEA, MAFF, MAFG, MAFK, MAGEA12, CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAP1, 10-March 7-March 8-Mar, MARK2, MASP1, MATK, MATR3, MATR3, SNHG4, MB, MBD5, MBNL1, MBOAT7, MCC, MCFD2, MCM9, MCOLN3, MCRS1, MDC1, MDGA2, MDH2, MDM2, ME1, MEAK7, MECR, MED4, MEF2A, MEF2B, BORCS8-MEF2B, MEF2BNB-MEF2B, MEF2B, MEF2BNB, MEF2C, MEF2D, MEGF10, ME11, MEIS2, MELK, MET, METTL13, METTL23, MFF, MFN2, MFSD2A, MGST3, MIB2, MICAL1, MICAL3, MICOS10, NBL1, MICOS10-NBL1, MID1, MINA, MINOS1-NBL1, MINOS1, MIOS, MIPOL1, MIS12, MKLN1, MKNK1, MKNK1, MOB3C, MLF2, MLH1, MMP17, MOBP, MOCS1, MOGS, MOK, MORF4L1, MPC1, MPC2, MPG, MP1, MPP1, MPP2, MPPE1, MPST, MRAS, MRO, MROH1, MROH7-TTC4, MROH7, MRPL14, MRPL24, MRPL33, BABAM2, MRPL33, BRE, MRPL47, MRPL48, MRPL55, MRRF, MRTFA, MRTFB, MRVI1, MS4A1, MS4A15, MS4A3, MS4A6E, MS4A7, MS4A14, MSANTD3, MSANTD4, MSH5, MSH5-SAPCD1, MSL2, MSRB3, MSS51, MTCP1, CMC4, MTERF, MTERF1, MTERF3, MTERFD2, MTERFD3, MTF2, MTG2, MTHFD2, MTHFD2L, MTIF2, MTIF3, MTMR10, MTRF1, MTRR, MTUS2, MUTYH, MVK, MX1, MX2, MYH10, MYL12A, MYB, MYD88, MYL5, MYLIP, MYNN, MYO15A, MYO1B, MYOM2, MZF1, N4BP2L2, NAA60, NAB1, NAE1, NAGK, NAP1L1, NAP1L4, NAPG, NARFL, NARG2, NAT1, NAT10, NBPF11, WI2-3658N16.1, NBPF12, NBPF15, NBPF24, NBPF6, NBPF9, NBR1, NCAPG2, NCBP2, NCEH1, NCOA1, NCOA4, NDC1, NDRG1, NDRG2, NDRG4, NDST1, NDUFAF6, NDUFB2, NDUFC1, NDUFS1, NDUFS8, NDUFV1, NEDD1, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRAS1, NKX2-1, NLRC3, NME1, NME1-NME2, NME2, NME1-NME2, NME2, NME4, NME6, NME9, NOD1, NOL10, NOL8, NONO, NPAS1, NPIPA8, RP11-1212A22.1, NPIPB3, NPIPB4, NPIPB9, NPL, NPM1, NPPA, NQO2, NR1H3, NR2C2, NR2F2, NR4A1, NRDC, NREP, NRF1, NRG4, NRIP1, NSD2, NSDHL, NSG1, NSMCE2, NSRP1, NT5C2, NTF4, NTMT1, NTNG2, NUBP2, NUCB2, NUDT1, NUDT2, NUDT4, NUF2, NUMBL, NUP50, NUP54, NUP85, NVL, NXF1, NXPE1, NXPE3, OARD1, OAT, OAZ2, OCIAD1, OCLN, ODF2, OGDHL, OGFOD2, AC026362.1, OGFOD2, RP11-197N18.2, OLA1, OPRL1, OPTN, OR2H1, ORAI2, ORMDL1, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAK1, PAK3, PAK4, PAK7, PALB2, PANK2, PAQR6, PARP11, PARVG, PASK, PAX6, PBRM1, PBXIP1, PCBP3, PCBP4, AC115284.1, PCBP4, RP11-155D18.14, RP11-155D18.12, PCGF3, PCGF5, PCNP, PCSK9, PDCD10, PDCD6, AHRR, PDDC1, PDGFRB, PDIA6, PDIK1L, PDLIM7, PDP1, PDPK1, PDPN, PDZD11, PEA15, PEX2, PEX5, PEX5L, PFKM, PFN4, PGAP2, PGAP2, AC090587.2, PGAP3, PGM3, PGPEP1, PHB, PHC2, PHF20, PHF21A, PHF23, PHKB, PHLDB1, PHOSPHO1, PHOSPHO2, KLHL23, PI4 KB, PIAS2, PICALM, PIF1, PIGN, PIGO, PIGT, PIK3CD, PILRB, STAG3L5P-PVRIG2P-PILRB, PIP5KIB, PIR, PISD, PIWIL4, FUT4, PKD2, PKIA, PKIG, PKM, PKN2, PLAIA, PLA2G2A, PLA2G5, PLA2G7, PLAC8, PLAGL1, PLD1, PLD3, PLEKHA1, PLEKHA2, PLEKHA6, PLEKHG5, PLIN1, PLS1, PLS3, PLSCR1, PLSCR2, PLSCR4, PLXNB1, PLXNB2, PMP22, PMS1, PNISR, PNKP, AKTIS1, PNMT, PNPLA4, PNPLA8, PNPO, PNRC1, POCIB, POFUT1, POLB, POLD1, POLH, POL1, POLL, POLR1B, POM121, POM121C, AC006014.7, POM121C, AC211429.1, POMC, POMT1, POP1, PORCN, POU5F1, PSORSIC3, PPARD, PPARG, PPHLN1, PPIL3, PPIL4, PPMIA, PPM1B, AC013717.1, PPP1CB, PPP1R11, PPP1R13L, PPP1R26, PPP1R9A, PPP2R2B, PPP3CA, PPP6R1, PPP6R3, PPT2, PPT2-EGFL8, EGFL8, PPWD1, PRDM2, PRDM8, PRELID3A, PREPL, PRICKLE1, PRKAG1, PRMT2, PRMT5, PRMT7, PROM1, PRPS1, PRPSAP2, PRR14L, PRR15L, PRR5, PRR5-ARHGAP8, PRR5L, PRR7, PRRC2B, PRRT4, PRSS50, PRSS45, PRSS44, PRUNE, PRUNE1, PSEN1, PSMA2, PSMF1, PSORSIC1, PSPH, PSRC1, PTBP3, PTHLH, PTK2, PTPDC1, PTPRM, PUF60, PUM2, PUS1, PUS10, PXN, PXYLP1, PYCR1, QRICH1, R3HCC1L, R3HDM2, RAB17, RAB23, RAB3A, RAB3D, TMEM205, RAB4B-EGLN2, EGLN2, AC008537.1, RAB5B, RAB7L1, RABL2A, RABL2B, RABL5, RACGAP1, RAD17, RAD51L3-RFFL, RAD51D, RAD52, RAE1, RAI14, RAI2, RALBP1, RAN, RANGAP1, RAPIA, RAPIB, RAPIGAP, RAPGEF4, RAPGEFL1, RASGRP2, RASSF1, RBCK1, RBM12B, RBM14, RBM4, RBM14-RBM4, RBM23, RBM4, RBM14-RBM4, RBM47, RBM7, AP002373.1, RBM7, RP11-212D19.4, RBMS2, RBMYIE, RBPJ, RBPMS, RBSN, RCBTB2, RCC1, RCC1, SNHG3, RCCD1, RECOL, RELL2, REPIN1, AC073111.3, REPIN1, ZNF775, RER1, RERE, RFWD3, RFX3, RGL2, RGMB, RGS11, RGS3, RGS5, AL592435.1, RHBDD1, RHNO1, TULP3, RHOC, AL603832.3, RHOC, RP11-426L16.10, RHOH, RIC8B, RIMKLB, RIN1, RIPK2, RIT1, RLIM, RNASE4, ANG, AL163636.6, RNASEK, RNASEK-C17orf49, RNF111, RNF123, RNF13, RNF14, RNF185, RNF216, RNF24, RNF32, RNF34, RNF38, RNF4, RNF44, RNH1, RNMT, RNPS1, RO60, ROPN1, ROPN1B, ROR2, RP1-102H19.8, C6orf163, RP1-283E3.8, CDK11A, RP11-120M18.2, PRKARIA, RP11-133K1.2, PAK6, RP11-164J13.1, CAPN3, RP11-21J18.1, ANKRD12, RP11-322E11.6, INO80C, RP11-337€18.10, CHD1L, RP11-432B6.3, TRIM59, RP11-468E2.4, IRF9, RP11-484M3.5, UPK1B, RP11-517H2.6, CCR6, RP11-613M10.9, SLC25A51, RP11-659G9.3, RAB30, RP11-691N7.6, CTNND1, RP11-849H4.2, RP11-896J10.3, NKX2-1, RP11-96020.4, SQRDL, RP11-986E7.7, SERPINA3, RP4-769N13.6, GPRASP1, RP4-769N13.6, GPRASP2, RP4-798P15.3, SEC16B, RP5-1021120.4, ZNF410, RP6-109B7.3, FLJ27365, RPE, RPH3AL, RPL15, RPL17, RPL17-C18orf32, RPL17, RPL23A, RPL36, HSD11B1L, RPP38, RPS20, RPS27A, RPS3A, RPS6KA3, RPS6KC1, RPS6KL1, RPUSD1, RRAGD, RRAS2, RRBP1, RSLID1, RSRC2, RSRP1, RUBCNL, RUNXIT1, RUVBL2, RWDD1, RWDD4, S100A13, AL162258.1, S100A13, RP1-178F15.5, S100A16, S100A4, S100A3, S100A6, S100PBP, SAA1, SACMIL, SAMD4B, SARIA, SARAF, SARNP, RP11-76217.5, SCAMP5, SCAP, SCAPER, SCFD1, SCGB3A2, SCIN, SCML1, SONN1D, SCO2, SCOC, SCRN1, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPP1, 11-September 2-Sep, SERGEF, AC055860.1, SERP1, SERPINA1, SERPINA5, SERPINB6, SERPING 1, SERPINH1, SERTAD3, SETD5, SFMBT1, AC096887.1, SFTPA1, SFTPA2, SFXN2, SGCD, SGCE, SGK3, SGK3, C8orf44, SH2B1, SH2D6, SH3BP1, Z83844.3, SH3BP2, SH3BP5, SH3D19, SH3YL1, SHC1, SHISA5, SHMT1, SHMT2, SHOC2, SHROOM1, SIGLEC5, SIGLEC14, SIL1, SIN3A, SIRT2, SIRT6, SKP1, STAT4, AC104109.3, SLAIN1, SLC10A3, SLC12A9, SLC14A1, SLC16A6, SLC1A2, SLC1A6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A1, SLC2A14, SLC2A5, SLC2A8, SLC35B2, SLC35B3, SLC35C2, SLC37A1, SLC38A1, SLC38A11, SLC39A13, SLC39A14, SLC41A3, SLC44A3, SLC4A7, SLC4A8, SLC5A10, SLC5A11, SLC6A1, SLC6A12, SLC6A9, SLC7A2, SLC7A6, SLC7A7, SLCO1A2, SLCO1C1, SLCO2B1, SLFN11, SLFN12, SLFNL1, SLMO1, SLTM, SLU7, SMAD2, SMAP 2, SMARCA2, SMARCE1, AC073508.2, SMARCE1, KRT222, SMC6, SMG7, SMIM22, SMOX, SMPDL3A, SMTN, SMU1, SMUG1, SNAP25, SNCA, SNRK, SNRPC, SNRPD1, SNRPD2, SNRPN, SNRPN, SNURF, SNUPN, SNX11, SNX16, SNX17, SOAT1, SOHLH2, CCDC169-SOHLH2, CCDC169, SORBS1, SORBS2, SOX5, SP2, SPART, SPATA20, SPATA21, SPATS2, SPATS2L, SPDYE2, SPECC1, SPECC1L, SPECC1L-ADORA2A, SPECC1L-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SRC, SRGAP1, SRP68, SRSF11, SSX1, SSX2IP, ST3GAL4, ST3GAL6, ST5, ST6GALNAC6, ST7L, STAC3, STAG1, STAG2, STAMBP, STAMBPL1, STARD3NL, STAT6, STAU1, STAU2, AC022826.2, STAU2, RP11-463D19.2, STEAP2, STEAP3, STIL, STK25, STK33, STK38L, STK40, STMN1, STON1, STON1-GTF2AIL, STRAP, STRBP, STRC, AC011330.5, STRC, CATSPER2, STRC, CATSPER2, AC011330.5, STRC, STRCP1, STT3A, STX16-NPEPL1, NPEPL1, STX5, STX6, STX8, STXBP6, STYK1, SULT1A1, SULT1A2, SUMF2, SUN1, SUN2, SUN2, DNAL4, SUOX, SUPT6H, SUV39H2, SV2B, SYBU, SYNCRIP, SYNJ2, SYT1, SYTL4, TAB2, TACC1, TADA2B, TAFIC, TAF6, AC073842.2, TAF6, RP11-506M12.1, TAF9, TAGLN, TANK, TAPSAR1, PSMB9, TAPT1, TATDN1, TAZ, TBC1D1, TBC1D12, HELLS, TBC1D15, TBC1D3H, TBC ID3G, TBC1D5, TBC1D5, SATB1, TBCA, TBCEL, TBCEL, AP000646.1, TBLIXR1, TBP, TBX5, TBXAS1, TCAF1, TCEA2, TCEAL4, TCEAL8, TCEAL9, TCEANC, TCEB1, TCF19, TCF25, TCF4, TCP1, TCP10L, AP000275.65, TCP11, TCP11L2, TCTN1, TDG, TDP1, TDRD7, TEAD2, TECR, TENC1, TENT4A, TEX264, TEX30, TEX37, TFDP1, TFDP2, TFEB, TFG, TFP1, TF, TFP1, TGIF1, THAP6, THBS3, THOC5, THRAP3, THUMPD3, TIAL1, TIMM9, TIMP1, TIRAP, TJAP1, TJP2, TK2, TLDC1, TLE3, TLE6, TLN1, TLR10, TM9SF1, TMBIM1, TMBIM4, TMBIM6, TMC6, TMCC1, TMCO4, TMEM126A, TMEM139, TMEM150B, TMEM155, TMEM161B, TMEM164, TMEM168, TMEM169, TMEM175, TMEM176B, TMEM182, TMEM199, CTB-96E2.3, TMEM216, TMEM218, TMEM230, TMEM263, TMEM45A, TMEM45B, TMEM62, TMEM63B, TMEM66, TMEM68, TMEM9B, TMEM9B, TMPRSS11D, TMPRSS5, TMSB15B, TMTC4, TMUB2, TMX2-CTNND1, RP11-69 IN7.6, CTNND1, TNFAIP2, TNFAIP8L2, SCNM1, TNFRSF10C, TNFRSF19, TNFRSF8, TNFSF12-TNFSF13, TNFSF12, TNFSF13, TNFSF12-TNFSF13, TNFSF13, TNIP1, TNK2, TNNT1, TNRC18, TNS3, TOB2, TOM1L1, TOPIMT, TOP3B, TOX2, TP53, RP11-199F11.2, TP53111, TP53INP2, TPCN1, TPM3P9, AC022137.3, TPT1, TRA2B, TRAF2, TRAF3, TRAPPC12, TRAPPC3, TREH, TREX1, TREX2, TRIB2, TRIM3, TRIM36, TRIM39, TRIM46, TRIM6, TRIM6-TRIM34, TRIM6-TRIM34, TRIM34, TRIM66, TRIM73, TRIT1, TRMT10B, TRMT2B, TRMT2B-AS1, TRNT1, TRO, TROVE2, TRPS1, TRPT1, TSC2, TSGA10, TSPAN14, TSPAN3, TSPAN4, TSPAN5, TSPAN6, TSPAN9, TSPO, TTC12, TTC23, TTC3, TTC39A, TTC39C, TTLL1, TTLL7, TTPAL, TUBD1, TWNK, TXNL4A, TXNL4B, TXNRD1, TYK2, U2AF1, UBA2, UBA52, UBAP2, UBE2D2, UBE2D3, UBE2E3, UBE21, UBE2J2, UBE3A, UBL7, UBXN11, UBXN7, UGDH, UGGT1, UGP2, UMAD1, AC007161.3, UNC45A, UQCC1, URGCP-MRPS24, URGCP, USMG5, USP16, USP21, USP28, USP3, USP33, USP35, USP54, USP9Y, USPL1, UTP15, VARS2, VASH2, VAV3, VDAC1, VDAC2, VDR, VEZT, VGF, VIL1, VILL, VIPR1, VPS29, VPS37C, VPS8, VPS9D1, VRK2, VWA1, VWASA, WARS, WASF1, WASHC5, WBP5, WDHD1, WDPCP, WDR37, WDR53, WDR6, WDR72, WDR74, WDR81, WDR86, WDYHV1, WFDC3, WHSC1, WIPF1, WSCD2, WWP2, XAGEIA, XAGEIB, XKR9, XPNPEP1, XRCC3, XRN2, XXYLT1, YIF1A, YIF1B, YIPF1, YIPF5, YPEL5, YWHAB, YWHAZ, YY1AP1, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHC11, ZDHHC13, ZEB2, ZFAND5, ZFAND6, ZFP1, ZFP62, ZFX, ZFYVE16, ZFYVE19, ZFYVE20, ZFYVE27, ZHX2, AC016405.1, ZHX3, ZIK1, ZIM2, PEG3, ZKSCAN1, ZKSCAN3, ZKSCAN8, ZMAT3, ZMAT5, ZMIZ2, ZMYM6, ZMYND11, ZNF10, AC026786.1, ZNF133, ZNF146, ZNF16, ZNF177, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28, ZNF468, ZNF283, ZNF287, ZNF3, ZNF320, ZNF322, ZNF324B, ZNF331, ZNF334, ZNF34, ZNF350, ZNF385A, ZNF395, FBXO16, ZNF415, ZNF418, ZNF43, ZNF433-AS1, AC008770.4, ZNF438, ZNF444, ZNF445, ZNF467, ZNF480, ZNF493, ZNF493, CTD-2561J22.3, ZNF502, ZNF507, ZNF512, AC074091.1, ZNF512, RP11-158113.2, ZNF512B, ZNF512B, SAMD10, ZNF521, ZNF532, ZNF544, AC020915.5, ZNF544, CTD-3138B18.4, ZNF559, ZNF177, ZNF562, ZNF567, ZNF569, ZNF570, ZNF571-AS1, ZNF540, ZNF577, ZNF580, ZNF581, ZNF580, ZNF581, CCDC106, ZNF600, ZNF611, ZNF613, ZNF615, ZNF619, ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the MYB gene. In some embodiments, the gene encoding a target sequence comprises the SMN2 gene. In some embodiments, the gene encoding a target sequence comprises the FOAM1 gene.

Exemplary genes that may be modulated by the compounds of Formula (I) or (II) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu (SEQ ID NO: 1), AAAguaaaaa (SEQ ID NO: 2), AAAguaaaau (SEQ ID NO: 3), AAAguaaagu (SEQ ID NO: 4), AAAguaaaua (SEQ ID NO: 5), AAAguaaaug (SEQ ID NO: 6), AAAguaaauu (SEQ ID NO: 7), AAAguaacac (SEQ ID NO: 8), AAAguaacca (SEQ ID NO: 9), AAAguaacuu (SEQ ID NO: 10), AAAguaagaa (SEQ ID NO: 11), AAAguaagac (SEQ ID NO: 12), AAAguaagag (SEQ ID NO: 13), AAAguaagau (SEQ ID NO: 14), AAAguaagca (SEQ ID NO: 15), AAAguaagcc (SEQ ID NO: 16), AAAguaagcu (SEQ ID NO: 17), AAAguaagga (SEQ ID NO: 18), AAAguaaggg (SEQ ID NO: 19), AAAguaaggu (SEQ ID NO: 20), AAAguaagua (SEQ ID NO: 21), AAAguaaguc (SEQ ID NO: 22), AAAguaagug (SEQ ID NO: 23), AAAguaaguu (SEQ ID NO: 24), AAAguaaucu (SEQ ID NO: 25), AAAguaauua (SEQ ID NO: 26), AAAguacaaa (SEQ ID NO: 27), AAAguaccgg (SEQ ID NO: 28), AAAguacuag (SEQ ID NO: 29), AAAguacugg (SEQ ID NO: 30), AAAguacuuc (SEQ ID NO: 31), AAAguacuug (SEQ ID NO: 32), AAAguagcuu (SEQ ID NO: 33), AAAguaggag (SEQ ID NO: 34), AAAguaggau (SEQ ID NO: 35), AAAguagggg (SEQ ID NO: 36), AAAguaggua (SEQ ID NO: 37), AAAguaguaa (SEQ ID NO: 38), AAAguauauu (SEQ ID NO: 39), AAAguauccu (SEQ ID NO: 40), AAAguaucuc (SEQ ID NO: 41), AAAguaugga (SEQ ID NO: 42), AAAguaugua (SEQ ID NO: 43), AAAguaugug (SEQ ID NO: 44), AAAguauguu (SEQ ID NO: 45), AAAguauugg (SEQ ID NO: 46), AAAguauuuu (SEQ ID NO: 47), AAAgucagau (SEQ ID NO: 48), AAAgucugag (SEQ ID NO: 49), AAAgugaaua (SEQ ID NO: 50), AAAgugagaa (SEQ ID NO: 51), AAAgugagac (SEQ ID NO: 52), AAAgugagag (SEQ ID NO: 53), AAAgugagau (SEQ ID NO: 54), AAAgugagca (SEQ ID NO: 55), AAAgugagcu (SEQ ID NO: 56), AAAgugaggg (SEQ ID NO: 57), AAAgugagua (SEQ ID NO: 58), AAAgugaguc (SEQ ID NO: 59), AAAgugagug (SEQ ID NO: 60), AAAgugaguu (SEQ ID NO: 61), AAAgugcguc (SEQ ID NO: 62), AAAgugcuga (SEQ ID NO: 63), AAAguggguc (SEQ ID NO: 64), AAAguggguu (SEQ ID NO: 65), AAAgugguaa (SEQ ID NO: 66), AAAguguaug (SEQ ID NO: 67), AAAgugugug (SEQ ID NO: 68), AAAguguguu (SEQ ID NO: 69), AAAguuaagu (SEQ ID NO: 70), AAAguuacuu (SEQ ID NO: 71), AAAguuagug (SEQ ID NO: 72), AAAguuaugu (SEQ ID NO: 73), AAAguugagu (SEQ ID NO: 74), AAAguuugua (SEQ ID NO: 75), AACguaaaac (SEQ ID NO: 76), AACguaaagc (SEQ ID NO: 77), AACguaaagg (SEQ ID NO: 78), AACguaagca (SEQ ID NO: 79), AACguaaggg (SEQ ID NO: 80), AACguaaguc (SEQ ID NO: 81), AACguaagug (SEQ ID NO: 82), AACguaaugg (SEQ ID NO: 83), AACguaguga (SEQ ID NO: 84), AACguaugua (SEQ ID NO: 85), AACguauguu (SEQ ID NO: 86), AACgugagca (SEQ ID NO: 87), AACgugagga (SEQ ID NO: 88), AACgugauuu (SEQ ID NO: 89), AACgugggau (SEQ ID NO: 90), AACgugggua (SEQ ID NO: 91), AACguguguu (SEQ ID NO: 92), AACguuggua (SEQ ID NO: 93), AAGgcaaauu (SEQ ID NO: 94), AAGgcaagag (SEQ ID NO: 95), AAGgcaagau (SEQ ID NO: 96), AAGgcaagcc (SEQ ID NO: 97), AAGgcaagga (SEQ ID NO: 98), AAGgcaaggg (SEQ ID NO: 99), AAGgcaagug (SEQ ID NO: 100), AAGgcaaguu (SEQ ID NO: 101), AAGgcacugc (SEQ ID NO: 102), AAGgcagaaa (SEQ ID NO: 103), AAGgcaggau (SEQ ID NO: 104), AAGgcaggca (SEQ ID NO: 105), AAGgcaggga (SEQ ID NO: 106), AAGgcagggg (SEQ ID NO: 107), AAGgcaggua (SEQ ID NO: 108), AAGgcaggug (SEQ ID NO: 109), AAGgcaucuc (SEQ ID NO: 110), AAGgcaugcu (SEQ ID NO: 111), AAGgcaugga (SEQ ID NO: 112), AAGgcauguu (SEQ ID NO: 113), AAGgcauuau (SEQ ID NO: 114), AAGgcgagcu (SEQ ID NO: 115), AAGgcgaguc (SEQ ID NO: 116), AAGgcgaguu (SEQ ID NO: 117), AAGgcuagcc (SEQ ID NO: 118), AAGguaaaaa (SEQ ID NO: 119), AAGguaaaac (SEQ ID NO: 120), AAGguaaaag (SEQ ID NO: 121), AAGguaaaau (SEQ ID NO: 122), AAGguaaaca (SEQ ID NO: 123), AAGguaaacc (SEQ ID NO: 124), AAGguaaacu (SEQ ID NO: 125), AAGguaaaga (SEQ ID NO: 126), AAGguaaagc (SEQ ID NO: 127), AAGguaaagg (SEQ ID NO: 128), AAGguaaagu (SEQ ID NO: 129), AAGguaaaua (SEQ ID NO: 130), AAGguaaauc (SEQ ID NO: 131), AAGguaaaug (SEQ ID NO: 132), AAGguaaauu (SEQ ID NO: 133), AAGguaacaa (SEQ ID NO: 134), AAGguaacau (SEQ ID NO: 135), AAGguaaccc (SEQ ID NO: 136), AAGguaacua (SEQ ID NO: 137), AAGguaacuc (SEQ ID NO: 138), AAGguaacug (SEQ ID NO: 139), AAGguaacuu (SEQ ID NO: 140), AAGguaagaa (SEQ ID NO: 141), AAGguaagac (SEQ ID NO: 142), AAGguaagag (SEQ ID NO: 143), AAGguaagau (SEQ ID NO: 144), AAGguaagca (SEQ ID NO: 145), AAGguaagcc (SEQ ID NO: 146), AAGguaagcg (SEQ ID NO: 147), AAGguaagcu (SEQ ID NO: 148), AAGguaagga (SEQ ID NO: 149), AAGguaaggc (SEQ ID NO: 150), AAGguaaggg (SEQ ID NO: 151), AAGguaaggu (SEQ ID NO: 152), AAGguaagua (SEQ ID NO: 153), AAGguaaguc (SEQ ID NO: 154), AAGguaagug (SEQ ID NO: 155), AAGguaaguu (SEQ ID NO: 156), AAGguaauaa (SEQ ID NO: 157), AAGguaauac (SEQ ID NO: 158), AAGguaauag (SEQ ID NO: 159), AAGguaauau (SEQ ID NO: 160), AAGguaauca (SEQ ID NO: 161), AAGguaaucc (SEQ ID NO: 162), AAGguaaucu (SEQ ID NO: 163), AAGguaauga (SEQ ID NO: 164), AAGguaaugc (SEQ ID NO: 165), AAGguaaugg (SEQ ID NO: 166), AAGguaaugu (SEQ ID NO: 167), AAGguaauua (SEQ ID NO: 168), AAGguaauuc (SEQ ID NO: 169), AAGguaauug (SEQ ID NO: 170), AAGguaauuu (SEQ ID NO: 171), AAGguacaaa (SEQ ID NO: 172), AAGguacaag (SEQ ID NO: 173), AAGguacaau (SEQ ID NO: 174), AAGguacacc (SEQ ID NO: 175), AAGguacacu (SEQ ID NO: 176), AAGguacagg (SEQ ID NO: 177), AAGguacagu (SEQ ID NO: 178), AAGguacaua (SEQ ID NO: 179), AAGguacaug (SEQ ID NO: 180), AAGguacauu (SEQ ID NO: 181), AAGguaccaa (SEQ ID NO: 182), AAGguaccag (SEQ ID NO: 183), AAGguaccca (SEQ ID NO: 184), AAGguacccu (SEQ ID NO: 185), AAGguaccuc (SEQ ID NO: 186), AAGguaccug (SEQ ID NO: 187), AAGguaccuu (SEQ ID NO: 188), AAGguacgaa (SEQ ID NO: 189), AAGguacggg (SEQ ID NO: 190), AAGguacggu (SEQ ID NO: 191), AAGguacguc (SEQ ID NO: 192), AAGguacguu (SEQ ID NO: 193), AAGguacuaa (SEQ ID NO: 194), AAGguacuau (SEQ ID NO: 195), AAGguacucu (SEQ ID NO: 196), AAGguacuga (SEQ ID NO: 197), AAGguacugc (SEQ ID NO: 198), AAGguacugu (SEQ ID NO: 199), AAGguacuuc (SEQ ID NO: 200), AAGguacuug (SEQ ID NO: 201), AAGguacuuu (SEQ ID NO: 202), AAGguagaaa (SEQ ID NO: 203), AAGguagaac (SEQ ID NO: 204), AAGguagaca (SEQ ID NO: 205), AAGguagacc (SEQ ID NO: 206), AAGguagacu (SEQ ID NO: 207), AAGguagagu (SEQ ID NO: 208), AAGguagaua (SEQ ID NO: 209), AAGguagcaa (SEQ ID NO: 210), AAGguagcag (SEQ ID NO: 211), AAGguagcca (SEQ ID NO: 212), AAGguagccu (SEQ ID NO: 213), AAGguagcua (SEQ ID NO: 214), AAGguagcug (SEQ ID NO: 215), AAGguagcuu (SEQ ID NO: 216), AAGguaggaa (SEQ ID NO: 217), AAGguaggag (SEQ ID NO: 218), AAGguaggau (SEQ ID NO: 219), AAGguaggca (SEQ ID NO: 220), AAGguaggcc (SEQ ID NO: 221), AAGguaggcu (SEQ ID NO: 222), AAGguaggga (SEQ ID NO: 223), AAGguagggc (SEQ ID NO: 224), AAGguagggg (SEQ ID NO: 225), AAGguagggu (SEQ ID NO: 226), AAGguaggua (SEQ ID NO: 227), AAGguagguc (SEQ ID NO: 228), AAGguaggug (SEQ ID NO: 229), AAGguagguu (SEQ ID NO: 230), AAGguaguaa (SEQ ID NO: 231), AAGguaguag (SEQ ID NO: 232), AAGguagucu (SEQ ID NO: 233), AAGguagugc (SEQ ID NO: 234), AAGguagugg (SEQ ID NO: 235), AAGguaguuc (SEQ ID NO: 236), AAGguaguuu (SEQ ID NO: 237), AAGguauaaa (SEQ ID NO: 238), AAGguauaau (SEQ ID NO: 239), AAGguauaca (SEQ ID NO: 240), AAGguauacu (SEQ ID NO: 241), AAGguauaua (SEQ ID NO: 242), AAGguauauc (SEQ ID NO: 243), AAGguauaug (SEQ ID NO: 244), AAGguauauu (SEQ ID NO: 245), AAGguaucac (SEQ ID NO: 246), AAGguaucag (SEQ ID NO: 247), AAGguauccc (SEQ ID NO: 248), AAGguauccu (SEQ ID NO: 249), AAGguaucuc (SEQ ID NO: 250), AAGguaucug (SEQ ID NO: 251), AAGguaucuu (SEQ ID NO: 252), AAGguaugaa (SEQ ID NO: 253), AAGguaugac (SEQ ID NO: 254), AAGguaugag (SEQ ID NO: 255), AAGguaugau (SEQ ID NO: 256), AAGguaugca (SEQ ID NO: 257), AAGguaugcc (SEQ ID NO: 258), AAGguaugcu (SEQ ID NO: 259), AAGguaugga (SEQ ID NO: 260), AAGguauggc (SEQ ID NO: 261), AAGguauggg (SEQ ID NO: 262), AAGguaugua (SEQ ID NO: 263), AAGguauguc (SEQ ID NO: 264), AAGguaugug (SEQ ID NO: 265), AAGguauguu (SEQ ID NO: 266), AAGguauuaa (SEQ ID NO: 267), AAGguauuac (SEQ ID NO: 268), AAGguauuag (SEQ ID NO: 269), AAGguauuau (SEQ ID NO: 270), AAGguauucc (SEQ ID NO: 271), AAGguauuga (SEQ ID NO: 272), AAGguauugu (SEQ ID NO: 273), AAGguauuua (SEQ ID NO: 274), AAGguauuuc (SEQ ID NO: 275), AAGguauuug (SEQ ID NO: 276), AAGguauuuu (SEQ ID NO: 277), AAGgucaaau (SEQ ID NO: 278), AAGgucaaga (SEQ ID NO: 279), AAGgucaagu (SEQ ID NO: 280), AAGgucacag (SEQ ID NO: 281), AAGgucagaa (SEQ ID NO: 282), AAGgucagac (SEQ ID NO: 283), AAGgucagag (SEQ ID NO: 284), AAGgucagca (SEQ ID NO: 285), AAGgucagcc (SEQ ID NO: 286), AAGgucagcg (SEQ ID NO: 287), AAGgucagcu (SEQ ID NO: 288), AAGgucagga (SEQ ID NO: 289), AAGgucaggc (SEQ ID NO: 290), AAGgucaggg (SEQ ID NO: 291), AAGgucaggu (SEQ ID NO: 292), AAGgucagua (SEQ ID NO: 293), AAGgucaguc (SEQ ID NO: 294), AAGgucagug (SEQ ID NO: 295), AAGgucaguu (SEQ ID NO: 296), AAGgucauag (SEQ ID NO: 297), AAGgucaucu (SEQ ID NO: 298), AAGguccaca (SEQ ID NO: 299), AAGguccaga (SEQ ID NO: 300), AAGguccaua (SEQ ID NO: 301), AAGgucccag (SEQ ID NO: 302), AAGgucccuc (SEQ ID NO: 303), AAGguccuuc (SEQ ID NO: 304), AAGgucgagg (SEQ ID NO: 305), AAGgucuaau (SEQ ID NO: 306), AAGgucuacc (SEQ ID NO: 307), AAGgucuaua (SEQ ID NO: 308), AAGgucuccu (SEQ ID NO: 309), AAGgucucug (SEQ ID NO: 310), AAGgucucuu (SEQ ID NO: 311), AAGgucugaa (SEQ ID NO: 312), AAGgucugag (SEQ ID NO: 313), AAGgucugga (SEQ ID NO: 314), AAGgucuggg (SEQ ID NO: 315), AAGgucugua (SEQ ID NO: 316), AAGgucuguu (SEQ ID NO: 317), AAGgucuucu (SEQ ID NO: 318), AAGgucuuuu (SEQ ID NO: 319), AAGgugaaac (SEQ ID NO: 320), AAGgugaaag (SEQ ID NO: 321), AAGgugaaau (SEQ ID NO: 322), AAGgugaacu (SEQ ID NO: 323), AAGgugaagc (SEQ ID NO: 324), AAGgugaagg (SEQ ID NO: 325), AAGgugaagu (SEQ ID NO: 326), AAGgugaaua (SEQ ID NO: 327), AAGgugaaug (SEQ ID NO: 328), AAGgugaauu (SEQ ID NO: 329), AAGgugacaa (SEQ ID NO: 330), AAGgugacag (SEQ ID NO: 331), AAGgugacau (SEQ ID NO: 332), AAGgugacug (SEQ ID NO: 333), AAGgugacuu (SEQ ID NO: 334), AAGgugagaa (SEQ ID NO: 335), AAGgugagac (SEQ ID NO: 336), AAGgugagag (SEQ ID NO: 337), AAGgugagau (SEQ ID NO: 338), AAGgugagca (SEQ ID NO: 339), AAGgugagcc (SEQ ID NO: 340), AAGgugagcg (SEQ ID NO: 341), AAGgugagcu (SEQ ID NO: 342), AAGgugagga (SEQ ID NO: 343), AAGgugaggc (SEQ ID NO: 344), AAGgugaggg (SEQ ID NO: 345), AAGgugaggu (SEQ ID NO: 346), AAGgugagua (SEQ ID NO: 347), AAGgugaguc (SEQ ID NO: 348), AAGgugagug (SEQ ID NO: 349), AAGgugaguu (SEQ ID NO: 350), AAGgugauaa (SEQ ID NO: 351), AAGgugauca (SEQ ID NO: 352), AAGgugaucc (SEQ ID NO: 353), AAGgugauga (SEQ ID NO: 354), AAGgugaugc (SEQ ID NO: 355), AAGgugaugu (SEQ ID NO: 356), AAGgugauua (SEQ ID NO: 357), AAGgugauug (SEQ ID NO: 358), AAGgugauuu (SEQ ID NO: 359), AAGgugcaca (SEQ ID NO: 360), AAGgugcauc (SEQ ID NO: 361), AAGgugcccu (SEQ ID NO: 362), AAGgugccug (SEQ ID NO: 363), AAGgugcgug (SEQ ID NO: 364), AAGgugcguu (SEQ ID NO: 365), AAGgugcucc (SEQ ID NO: 366), AAGgugcuga (SEQ ID NO: 367), AAGgugcugc (SEQ ID NO: 368), AAGgugcugg (SEQ ID NO: 369), AAGgugcuua (SEQ ID NO: 370), AAGgugcuuu (SEQ ID NO: 371), AAGguggaua (SEQ ID NO: 372), AAGguggcua (SEQ ID NO: 373), AAGguggcug (SEQ ID NO: 374), AAGguggcuu (SEQ ID NO: 375), AAGgugggaa (SEQ ID NO: 376), AAGgugggag (SEQ ID NO: 377), AAGgugggau (SEQ ID NO: 378), AAGgugggca (SEQ ID NO: 379), AAGgugggcc (SEQ ID NO: 380), AAGgugggcg (SEQ ID NO: 381), AAGgugggga (SEQ ID NO: 382), AAGguggggu (SEQ ID NO: 383), AAGgugggua (SEQ ID NO: 384), AAGgugggug (SEQ ID NO: 385), AAGguggguu (SEQ ID NO: 386), AAGgugguaa (SEQ ID NO: 387), AAGgugguac (SEQ ID NO: 388), AAGgugguau (SEQ ID NO: 389), AAGguggugg (SEQ ID NO: 390), AAGgugguua (SEQ ID NO: 391), AAGgugguuc (SEQ ID NO: 392), AAGgugguuu (SEQ ID NO: 393), AAGguguaag (SEQ ID NO: 394), AAGgugucaa (SEQ ID NO: 395), AAGgugucag (SEQ ID NO: 396), AAGgugucug (SEQ ID NO: 397), AAGgugugaa (SEQ ID NO: 398), AAGgugugag (SEQ ID NO: 399), AAGgugugca (SEQ ID NO: 400), AAGgugugga (SEQ ID NO: 401), AAGguguggu (SEQ ID NO: 402), AAGgugugua (SEQ ID NO: 403), AAGguguguc (SEQ ID NO: 404), AAGgugugug (SEQ ID NO: 405), AAGguguguu (SEQ ID NO: 406), AAGguguucu (SEQ ID NO: 407), AAGguguugc (SEQ ID NO: 408), AAGguguugg (SEQ ID NO: 409), AAGguguuug (SEQ ID NO: 410), AAGguuaaaa (SEQ ID NO: 411), AAGguuaaca (SEQ ID NO: 412), AAGguuaagc (SEQ ID NO: 413), AAGguuaauu (SEQ ID NO: 414), AAGguuacau (SEQ ID NO: 415), AAGguuagaa (SEQ ID NO: 416), AAGguuagau (SEQ ID NO: 417), AAGguuagca (SEQ ID NO: 418), AAGguuagcc (SEQ ID NO: 419), AAGguuagga (SEQ ID NO: 420), AAGguuaggc (SEQ ID NO: 421), AAGguuagua (SEQ ID NO: 422), AAGguuaguc (SEQ ID NO: 423), AAGguuagug (SEQ ID NO: 424), AAGguuaguu (SEQ ID NO: 425), AAGguuauag (SEQ ID NO: 426), AAGguuauga (SEQ ID NO: 427), AAGguucaaa (SEQ ID NO: 428), AAGguucaag (SEQ ID NO: 429), AAGguuccuu (SEQ ID NO: 430), AAGguucggc (SEQ ID NO: 431), AAGguucguu (SEQ ID NO: 432), AAGguucuaa (SEQ ID NO: 433), AAGguucuga (SEQ ID NO: 434), AAGguucuua (SEQ ID NO: 435), AAGguugaau (SEQ ID NO: 436), AAGguugacu (SEQ ID NO: 437), AAGguugagg (SEQ ID NO: 438), AAGguugagu (SEQ ID NO: 439), AAGguugaua (SEQ ID NO: 440), AAGguugcac (SEQ ID NO: 441), AAGguugcug (SEQ ID NO: 442), AAGguuggaa (SEQ ID NO: 443), AAGguuggca (SEQ ID NO: 444), AAGguuggga (SEQ ID NO: 445), AAGguugggg (SEQ ID NO: 446), AAGguuggua (SEQ ID NO: 447), AAGguugguc (SEQ ID NO: 448), AAGguuggug (SEQ ID NO: 449), AAGguugguu (SEQ ID NO: 450), AAGguuguaa (SEQ ID NO: 451), AAGguugucc (SEQ ID NO: 452), AAGguugugc (SEQ ID NO: 453), AAGguuguua (SEQ ID NO: 454), AAGguuuacc (SEQ ID NO: 455), AAGguuuaua (SEQ ID NO: 456), AAGguuuauu (SEQ ID NO: 457), AAGguuuccu (SEQ ID NO: 458), AAGguuucgu (SEQ ID NO: 459), AAGguuugag (SEQ ID NO: 460), AAGguuugca (SEQ ID NO: 461), AAGguuugcc (SEQ ID NO: 462), AAGguuugcu (SEQ ID NO: 463), AAGguuugga (SEQ ID NO: 464), AAGguuuggu (SEQ ID NO: 465), AAGguuugua (SEQ ID NO: 466), AAGguuuguc (SEQ ID NO: 467), AAGguuugug (SEQ ID NO: 468), AAGguuuuaa (SEQ ID NO: 469), AAGguuuuca (SEQ ID NO: 470), AAGguuuucg (SEQ ID NO: 471), AAGguuuugc (SEQ ID NO: 472), AAGguuuugu (SEQ ID NO: 473), AAGguuuuuu (SEQ ID NO: 474), AAUgcaagua (SEQ ID NO: 475), AAUgcaaguc (SEQ ID NO: 476), AAUguaaaca (SEQ ID NO: 477), AAUguaaaua (SEQ ID NO: 478), AAUguaaauc (SEQ ID NO: 479), AAUguaaaug (SEQ ID NO: 480), AAUguaaauu (SEQ ID NO: 481), AAUguaacua (SEQ ID NO: 482), AAUguaagaa (SEQ ID NO: 483), AAUguaagag (SEQ ID NO: 484), AAUguaagau (SEQ ID NO: 485), AAUguaagcc (SEQ ID NO: 486), AAUguaagcu (SEQ ID NO: 487), AAUguaagga (SEQ ID NO: 488), AAUguaagua (SEQ ID NO: 489), AAUguaaguc (SEQ ID NO: 490), AAUguaagug (SEQ ID NO: 491), AAUguaaguu (SEQ ID NO: 492), AAUguaauca (SEQ ID NO: 493), AAUguaauga (SEQ ID NO: 494), AAUguaaugu (SEQ ID NO: 495), AAUguacauc (SEQ ID NO: 496), AAUguacaug (SEQ ID NO: 497), AAUguacgau (SEQ ID NO: 498), AAUguacgua (SEQ ID NO: 499), AAUguacguc (SEQ ID NO: 500), AAUguacgug (SEQ ID NO: 501), AAUguacucu (SEQ ID NO: 502), AAUguaggca (SEQ ID NO: 503), AAUguagguu (SEQ ID NO: 504), AAUguaucua (SEQ ID NO: 505), AAUguaugaa (SEQ ID NO: 506), AAUguaugua (SEQ ID NO: 507), AAUguaugug (SEQ ID NO: 508), AAUguauguu (SEQ ID NO: 509), AAUgucagag (SEQ ID NO: 510), AAUgucagau (SEQ ID NO: 511), AAUgucagcu (SEQ ID NO: 512), AAUgucagua (SEQ ID NO: 513), AAUgucaguc (SEQ ID NO: 514), AAUgucagug (SEQ ID NO: 515), AAUgucaguu (SEQ ID NO: 516), AAUgucggua (SEQ ID NO: 517), AAUgucuguu (SEQ ID NO: 518), AAUgugagaa (SEQ ID NO: 519), AAUgugagca (SEQ ID NO: 520), AAUgugagcc (SEQ ID NO: 521), AAUgugagga (SEQ ID NO: 522), AAUgugagua (SEQ ID NO: 523), AAUgugaguc (SEQ ID NO: 524), AAUgugagug (SEQ ID NO: 525), AAUgugaguu (SEQ ID NO: 526), AAUgugauau (SEQ ID NO: 527), AAUgugcaua (SEQ ID NO: 528), AAUgugcgua (SEQ ID NO: 529), AAUgugcguc (SEQ ID NO: 530), AAUgugggac (SEQ ID NO: 531), AAUguggguc (SEQ ID NO: 532), AAUgugggug (SEQ ID NO: 533), AAUgugguuu (SEQ ID NO: 534), AAUgugugua (SEQ ID NO: 535), AAUguuaagu (SEQ ID NO: 536), AAUguuagaa (SEQ ID NO: 537), AAUguuagau (SEQ ID NO: 538), AAUguuagua (SEQ ID NO: 539), AAUguuggug (SEQ ID NO: 540), ACAgcaagua (SEQ ID NO: 541), ACAguaaaua (SEQ ID NO: 542), ACAguaaaug (SEQ ID NO: 543), ACAguaagaa (SEQ ID NO: 544), ACAguaagca (SEQ ID NO: 545), ACAguaagua (SEQ ID NO: 546), ACAguaaguc (SEQ ID NO: 547), ACAguaagug (SEQ ID NO: 548), ACAguaaguu (SEQ ID NO: 549), ACAguacgua (SEQ ID NO: 550), ACAguaggug (SEQ ID NO: 551), ACAguauaac (SEQ ID NO: 552), ACAguaugua (SEQ ID NO: 553), ACAgucaguu (SEQ ID NO: 554), ACAgugagaa (SEQ ID NO: 555), ACAgugagcc (SEQ ID NO: 556), ACAgugagcu (SEQ ID NO: 557), ACAgugagga (SEQ ID NO: 558), ACAgugaggu (SEQ ID NO: 559), ACAgugagua (SEQ ID NO: 560), ACAgugaguc (SEQ ID NO: 561), ACAgugagug (SEQ ID NO: 562), ACAgugaguu (SEQ ID NO: 563), ACAgugggua (SEQ ID NO: 564), ACAguggguu (SEQ ID NO: 565), ACAguguaaa (SEQ ID NO: 566), ACAguuaagc (SEQ ID NO: 567), ACAguuaagu (SEQ ID NO: 568), ACAguuaugu (SEQ ID NO: 569), ACAguugagu (SEQ ID NO: 570), ACAguuguga (SEQ ID NO: 571), ACCguaagua (SEQ ID NO: 572), ACCgugagaa (SEQ ID NO: 573), ACCgugagca (SEQ ID NO: 574), ACCgugaguu (SEQ ID NO: 575), ACCgugggug (SEQ ID NO: 576), ACGguaaaac (SEQ ID NO: 577), ACGguaacua (SEQ ID NO: 578), ACGguaagua (SEQ ID NO: 579), ACGguaagug (SEQ ID NO: 580), ACGguaaguu (SEQ ID NO: 581), ACGguaauua (SEQ ID NO: 582), ACGguaauuu (SEQ ID NO: 583), ACGguacaau (SEQ ID NO: 584), ACGguacagu (SEQ ID NO: 585), ACGguaccag (SEQ ID NO: 586), ACGguacggu (SEQ ID NO: 587), ACGguacgua (SEQ ID NO: 588), ACGguaggaa (SEQ ID NO: 589), ACGguaggag (SEQ ID NO: 590), ACGguaggug (SEQ ID NO: 591), ACGguaguaa (SEQ ID NO: 592), ACGguauaau (SEQ ID NO: 593), ACGguaugac (SEQ ID NO: 594), ACGguaugcg (SEQ ID NO: 595), ACGguaugua (SEQ ID NO: 596), ACGguauguc (SEQ ID NO: 597), ACGgugaaac (SEQ ID NO: 598), ACGgugaagu (SEQ ID NO: 599), ACGgugaauc (SEQ ID NO: 600), ACGgugacag (SEQ ID NO: 601), ACGgugacca (SEQ ID NO: 602), ACGgugagaa (SEQ ID NO: 603), ACGgugagau (SEQ ID NO: 604), ACGgugagcc (SEQ ID NO: 605), ACGgugagua (SEQ ID NO: 606), ACGgugagug (SEQ ID NO: 607), ACGgugaguu (SEQ ID NO: 608), ACGgugcgug (SEQ ID NO: 609), ACGguggcac (SEQ ID NO: 610), ACGguggggc (SEQ ID NO: 611), ACGgugggug (SEQ ID NO: 612), ACGguguagu (SEQ ID NO: 613), ACGgugucac (SEQ ID NO: 614), ACGgugugua (SEQ ID NO: 615), ACGguguguu (SEQ ID NO: 616), ACGguuagug (SEQ ID NO: 617), ACGguuaguu (SEQ ID NO: 618), ACGguucaau (SEQ ID NO: 619), ACUguaaaua (SEQ ID NO: 620), ACUguaagaa (SEQ ID NO: 621), ACUguaagac (SEQ ID NO: 622), ACUguaagca (SEQ ID NO: 623), ACUguaagcu (SEQ ID NO: 624), ACUguaagua (SEQ ID NO: 625), ACUguaaguc (SEQ ID NO: 626), ACUguaaguu (SEQ ID NO: 627), ACUguacguu (SEQ ID NO: 628), ACUguacuge (SEQ ID NO: 629), ACUguaggcu (SEQ ID NO: 630), ACUguaggua (SEQ ID NO: 631), ACUguauauu (SEQ ID NO: 632), ACUguaugaa (SEQ ID NO: 633), ACUguaugcu (SEQ ID NO: 634), ACUguaugug (SEQ ID NO: 635), ACUguauucc (SEQ ID NO: 636), ACUgucagcu (SEQ ID NO: 637), ACUgucagug (SEQ ID NO: 638), ACUgugaacg (SEQ ID NO: 639), ACUgugagca (SEQ ID NO: 640), ACUgugagcg (SEQ ID NO: 641), ACUgugagcu (SEQ ID NO: 642), ACUgugagua (SEQ ID NO: 643), ACUgugaguc (SEQ ID NO: 644), ACUgugagug (SEQ ID NO: 645), ACUgugaguu (SEQ ID NO: 646), ACUgugggua (SEQ ID NO: 647), ACUgugugug (SEQ ID NO: 648), ACUguuaagu (SEQ ID NO: 649), AGAgcaagua (SEQ ID NO: 650), AGAguaaaac (SEQ ID NO: 651), AGAguaaacg (SEQ ID NO: 652), AGAguaaaga (SEQ ID NO: 653), AGAguaaagu (SEQ ID NO: 654), AGAguaaauc (SEQ ID NO: 655), AGAguaaaug (SEQ ID NO: 656), AGAguaacau (SEQ ID NO: 657), AGAguaacua (SEQ ID NO: 658), AGAguaagaa (SEQ ID NO: 659), AGAguaagac (SEQ ID NO: 660), AGAguaagag (SEQ ID NO: 661), AGAguaagau (SEQ ID NO: 662), AGAguaagca (SEQ ID NO: 663), AGAguaagcu (SEQ ID NO: 664), AGAguaagga (SEQ ID NO: 665), AGAguaaggc (SEQ ID NO: 666), AGAguaaggg (SEQ ID NO: 667), AGAguaaggu (SEQ ID NO: 668), AGAguaaguc (SEQ ID NO: 669), AGAguaagug (SEQ ID NO: 670), AGAguaaguu (SEQ ID NO: 671), AGAguaauaa (SEQ ID NO: 672), AGAguaaugu (SEQ ID NO: 673), AGAguaauuc (SEQ ID NO: 674), AGAguaauuu (SEQ ID NO: 675), AGAguacacc (SEQ ID NO: 676), AGAguaccug (SEQ ID NO: 677), AGAguacgug (SEQ ID NO: 678), AGAguacucu (SEQ ID NO: 679), AGAguacuga (SEQ ID NO: 680), AGAguacuuu (SEQ ID NO: 681), AGAguagcug (SEQ ID NO: 682), AGAguaggaa (SEQ ID NO: 683), AGAguaggga (SEQ ID NO: 684), AGAguagggu (SEQ ID NO: 685), AGAguagguc (SEQ ID NO: 686), AGAguaggug (SEQ ID NO: 687), AGAguagguu (SEQ ID NO: 688), AGAguauaua (SEQ ID NO: 689), AGAguauauu (SEQ ID NO: 690), AGAguaugaa (SEQ ID NO: 691), AGAguaugac (SEQ ID NO: 692), AGAguaugau (SEQ ID NO: 693), AGAguauguc (SEQ ID NO: 694), AGAguaugug (SEQ ID NO: 695), AGAguauguu (SEQ ID NO: 696), AGAguauuaa (SEQ ID NO: 697), AGAguauuau (SEQ ID NO: 698), AGAgucagug (SEQ ID NO: 699), AGAgugagac (SEQ ID NO: 700), AGAgugagag (SEQ ID NO: 701), AGAgugagau (SEQ ID NO: 702), AGAgugagca (SEQ ID NO: 703), AGAgugagua (SEQ ID NO: 704), AGAgugaguc (SEQ ID NO: 705), AGAgugagug (SEQ ID NO: 706), AGAgugaguu (SEQ ID NO: 707), AGAgugcguc (SEQ ID NO: 708), AGAgugggga (SEQ ID NO: 709), AGAgugggug (SEQ ID NO: 710), AGAgugugug (SEQ ID NO: 711), AGAguguuuc (SEQ ID NO: 712), AGAguuagua (SEQ ID NO: 713), AGAguugaga (SEQ ID NO: 714), AGAguugagu (SEQ ID NO: 715), AGAguugguu (SEQ ID NO: 716), AGAguuugau (SEQ ID NO: 717), AGCguaagcu (SEQ ID NO: 718), AGCguaagug (SEQ ID NO: 719), AGCgugagcc (SEQ ID NO: 720), AGCgugagug (SEQ ID NO: 721), AGCguuguuc (SEQ ID NO: 722), AGGgcagagu (SEQ ID NO: 723), AGGgcagccu (SEQ ID NO: 724), AGGgcuagua (SEQ ID NO: 725), AGGguaaaga (SEQ ID NO: 726), AGGguaaaua (SEQ ID NO: 727), AGGguaaauc (SEQ ID NO: 728), AGGguaaauu (SEQ ID NO: 729), AGGguaacca (SEQ ID NO: 730), AGGguaacug (SEQ ID NO: 731), AGGguaacuu (SEQ ID NO: 732), AGGguaagaa (SEQ ID NO: 733), AGGguaagag (SEQ ID NO: 734), AGGguaagau (SEQ ID NO: 735), AGGguaagca (SEQ ID NO: 736), AGGguaagga (SEQ ID NO: 737), AGGguaaggc (SEQ ID NO: 738), AGGguaaggg (SEQ ID NO: 739), AGGguaagua (SEQ ID NO: 740), AGGguaaguc (SEQ ID NO: 741), AGGguaagug (SEQ ID NO: 742), AGGguaaguu (SEQ ID NO: 743), AGGguaauac (SEQ ID NO: 744), AGGguaauga (SEQ ID NO: 745), AGGguaauua (SEQ ID NO: 746), AGGguaauuu (SEQ ID NO: 747), AGGguacacc (SEQ ID NO: 748), AGGguacagu (SEQ ID NO: 749), AGGguacggu (SEQ ID NO: 750), AGGguaggac (SEQ ID NO: 751), AGGguaggag (SEQ ID NO: 752), AGGguaggca (SEQ ID NO: 753), AGGguaggcc (SEQ ID NO: 754), AGGguaggga (SEQ ID NO: 755), AGGguagggu (SEQ ID NO: 756), AGGguagguc (SEQ ID NO: 757), AGGguaggug (SEQ ID NO: 758), AGGguagguu (SEQ ID NO: 759), AGGguauaua (SEQ ID NO: 760), AGGguaugac (SEQ ID NO: 761), AGGguaugag (SEQ ID NO: 762), AGGguaugau (SEQ ID NO: 763), AGGguaugca (SEQ ID NO: 764), AGGguaugcu (SEQ ID NO: 765), AGGguauggg (SEQ ID NO: 766), AGGguauggu (SEQ ID NO: 767), AGGguaugua (SEQ ID NO: 768), AGGguauguc (SEQ ID NO: 769), AGGguaugug (SEQ ID NO: 770), AGGguauuac (SEQ ID NO: 771), AGGguauucu (SEQ ID NO: 772), AGGguauuuc (SEQ ID NO: 773), AGGgucagag (SEQ ID NO: 774), AGGgucagca (SEQ ID NO: 775), AGGgucagga (SEQ ID NO: 776), AGGgucaggg (SEQ ID NO: 777), AGGgucagug (SEQ ID NO: 778), AGGgucaguu (SEQ ID NO: 779), AGGguccccu (SEQ ID NO: 780), AGGgucggga (SEQ ID NO: 781), AGGgucugca (SEQ ID NO: 782), AGGgucuguu (SEQ ID NO: 783), AGGgugaaga (SEQ ID NO: 784), AGGgugacua (SEQ ID NO: 785), AGGgugagaa (SEQ ID NO: 786), AGGgugagac (SEQ ID NO: 787), AGGgugagag (SEQ ID NO: 788), AGGgugagca (SEQ ID NO: 789), AGGgugagcc (SEQ ID NO: 790), AGGgugagcu (SEQ ID NO: 791), AGGgugagga (SEQ ID NO: 792), AGGgugaggg (SEQ ID NO: 793), AGGgugaggu (SEQ ID NO: 794), AGGgugagua (SEQ ID NO: 795), AGGgugaguc (SEQ ID NO: 796), AGGgugagug (SEQ ID NO: 797), AGGgugaguu (SEQ ID NO: 798), AGGgugggga (SEQ ID NO: 799), AGGguggggu (SEQ ID NO: 800), AGGgugggua (SEQ ID NO: 801), AGGgugggug (SEQ ID NO: 802), AGGgugugua (SEQ ID NO: 803), AGGgugugug (SEQ ID NO: 804), AGGguuaaug (SEQ ID NO: 805), AGGguuagaa (SEQ ID NO: 806), AGGguuaguu (SEQ ID NO: 807), AGGguuggug (SEQ ID NO: 808), AGGguuugug (SEQ ID NO: 809), AGGguuuguu (SEQ ID NO: 810), AGUguaaaag (SEQ ID NO: 811), AGUguaaaua (SEQ ID NO: 812), AGUguaaauu (SEQ ID NO: 813), AGUguaagaa (SEQ ID NO: 814), AGUguaagag (SEQ ID NO: 815), AGUguaagau (SEQ ID NO: 816), AGUguaagca (SEQ ID NO: 817), AGUguaagcc (SEQ ID NO: 818), AGUguaagua (SEQ ID NO: 819), AGUguaagug (SEQ ID NO: 820), AGUguaaguu (SEQ ID NO: 821), AGUguaauug (SEQ ID NO: 822), AGUguaggac (SEQ ID NO: 823), AGUguagguc (SEQ ID NO: 824), AGUguaugag (SEQ ID NO: 825), AGUguaugua (SEQ ID NO: 826), AGUguauguu (SEQ ID NO: 827), AGUguauugu (SEQ ID NO: 828), AGUguauuua (SEQ ID NO: 829), AGUgucaguc (SEQ ID NO: 830), AGUgugagag (SEQ ID NO: 831), AGUgugagca (SEQ ID NO: 832), AGUgugagcc (SEQ ID NO: 833), AGUgugagcu (SEQ ID NO: 834), AGUgugagua (SEQ ID NO: 835), AGUgugaguc (SEQ ID NO: 836), AGUgugagug (SEQ ID NO: 837), AGUgugaguu (SEQ ID NO: 838), AGUgugggua (SEQ ID NO: 839), AGUgugggug (SEQ ID NO: 840), AGUgugugua (SEQ ID NO: 841), AGUguuccua (SEQ ID NO: 842), AGUguugggg (SEQ ID NO: 843), AGUguuucag (SEQ ID NO: 844), AUAguaaaua (SEQ ID NO: 845), AUAguaagac (SEQ ID NO: 846), AUAguaagau (SEQ ID NO: 847), AUAguaagca (SEQ ID NO: 848), AUAguaagua (SEQ ID NO: 849), AUAguaagug (SEQ ID NO: 850), AUAguaaguu (SEQ ID NO: 851), AUAguaggua (SEQ ID NO: 852), AUAguauguu (SEQ ID NO: 853), AUAgucucac (SEQ ID NO: 854), AUAgugagac (SEQ ID NO: 855), AUAgugagag (SEQ ID NO: 856), AUAgugagau (SEQ ID NO: 857), AUAgugagcc (SEQ ID NO: 858), AUAgugaggc (SEQ ID NO: 859), AUAgugagua (SEQ ID NO: 860), AUAgugaguc (SEQ ID NO: 861), AUAgugagug (SEQ ID NO: 862), AUAgugcguc (SEQ ID NO: 863), AUAgugugua (SEQ ID NO: 864), AUAguucagu (SEQ ID NO: 865), AUCguaagcc (SEQ ID NO: 866), AUCguaaguu (SEQ ID NO: 867), AUCguauucc (SEQ ID NO: 868), AUCgugagua (SEQ ID NO: 869), AUGgcaagcg (SEQ ID NO: 870), AUGgcaagga (SEQ ID NO: 871), AUGgcaaguu (SEQ ID NO: 872), AUGgcaggua (SEQ ID NO: 873), AUGgcaugug (SEQ ID NO: 874), AUGgcgccau (SEQ ID NO: 875), AUGgcuugug (SEQ ID NO: 876), AUGguaaaac (SEQ ID NO: 877), AUGguaaaau (SEQ ID NO: 878), AUGguaaacc (SEQ ID NO: 879), AUGguaaaga (SEQ ID NO: 880), AUGguaaaua (SEQ ID NO: 881), AUGguaaaug (SEQ ID NO: 882), AUGguaaauu (SEQ ID NO: 883), AUGguaacag (SEQ ID NO: 884), AUGguaacau (SEQ ID NO: 885), AUGguaacua (SEQ ID NO: 886), AUGguaacuc (SEQ ID NO: 887), AUGguaacuu (SEQ ID NO: 888), AUGguaagaa (SEQ ID NO: 889), AUGguaagac (SEQ ID NO: 890), AUGguaagag (SEQ ID NO: 891), AUGguaagau (SEQ ID NO: 892), AUGguaagca (SEQ ID NO: 893), AUGguaagcc (SEQ ID NO: 894), AUGguaagcu (SEQ ID NO: 895), AUGguaagga (SEQ ID NO: 896), AUGguaaggg (SEQ ID NO: 897), AUGguaagua (SEQ ID NO: 898), AUGguaaguc (SEQ ID NO: 899), AUGguaagug (SEQ ID NO: 900), AUGguaaguu (SEQ ID NO: 901), AUGguaauaa (SEQ ID NO: 902), AUGguaauau (SEQ ID NO: 903), AUGguaauga (SEQ ID NO: 904), AUGguaaugg (SEQ ID NO: 905), AUGguaauug (SEQ ID NO: 906), AUGguaauuu (SEQ ID NO: 907), AUGguacage (SEQ ID NO: 908), AUGguacauc (SEQ ID NO: 909), AUGguaccag (SEQ ID NO: 910), AUGguaccug (SEQ ID NO: 911), AUGguacgag (SEQ ID NO: 912), AUGguacggu (SEQ ID NO: 913), AUGguagauc (SEQ ID NO: 914), AUGguagcag (SEQ ID NO: 915), AUGguagcug (SEQ ID NO: 916), AUGguaggaa (SEQ ID NO: 917), AUGguaggau (SEQ ID NO: 918), AUGguaggca (SEQ ID NO: 919), AUGguaggcu (SEQ ID NO: 920), AUGguagggg (SEQ ID NO: 921), AUGguagggu (SEQ ID NO: 922), AUGguaggua (SEQ ID NO: 923), AUGguaggug (SEQ ID NO: 924), AUGguaguuu (SEQ ID NO: 925), AUGguauagu (SEQ ID NO: 926), AUGguauaua (SEQ ID NO: 927), AUGguaucag (SEQ ID NO: 928), AUGguaucuu (SEQ ID NO: 929), AUGguaugau (SEQ ID NO: 930), AUGguaugca (SEQ ID NO: 931), AUGguaugcc (SEQ ID NO: 932), AUGguaugcg (SEQ ID NO: 933), AUGguaugcu (SEQ ID NO: 934), AUGguaugga (SEQ ID NO: 935), AUGguauggc (SEQ ID NO: 936), AUGguaugug (SEQ ID NO: 937), AUGguauguu (SEQ ID NO: 938), AUGguauuau (SEQ ID NO: 939), AUGguauuga (SEQ ID NO: 940), AUGguauuug (SEQ ID NO: 941), AUGgucaggg (SEQ ID NO: 942), AUGgucaguc (SEQ ID NO: 943), AUGgucagug (SEQ ID NO: 944), AUGgucauuu (SEQ ID NO: 945), AUGgugaaaa (SEQ ID NO: 946), AUGgugaaac (SEQ ID NO: 947), AUGgugaaau (SEQ ID NO: 948), AUGgugaacu (SEQ ID NO: 949), AUGgugaaga (SEQ ID NO: 950), AUGgugacgu (SEQ ID NO: 951), AUGgugagaa (SEQ ID NO: 952), AUGgugagac (SEQ ID NO: 953), AUGgugagag (SEQ ID NO: 954), AUGgugagca (SEQ ID NO: 955), AUGgugagcc (SEQ ID NO: 956), AUGgugageg (SEQ ID NO: 957), AUGgugagcu (SEQ ID NO: 958), AUGgugaggc (SEQ ID NO: 959), AUGgugaggg (SEQ ID NO: 960), AUGgugagua (SEQ ID NO: 961), AUGgugaguc (SEQ ID NO: 962), AUGgugagug (SEQ ID NO: 963), AUGgugaguu (SEQ ID NO: 964), AUGgugauuu (SEQ ID NO: 965), AUGgugcgau (SEQ ID NO: 966), AUGgugcgug (SEQ ID NO: 967), AUGgugggua (SEQ ID NO: 968), AUGgugggug (SEQ ID NO: 969), AUGguggguu (SEQ ID NO: 970), AUGgugguua (SEQ ID NO: 971), AUGguguaag (SEQ ID NO: 972), AUGgugugaa (SEQ ID NO: 973), AUGgugugua (SEQ ID NO: 974), AUGgugugug (SEQ ID NO: 975), AUGguuacuc (SEQ ID NO: 976), AUGguuagca (SEQ ID NO: 977), AUGguuaguc (SEQ ID NO: 978), AUGguuagug (SEQ ID NO: 979), AUGguuaguu (SEQ ID NO: 980), AUGguucagu (SEQ ID NO: 981), AUGguucguc (SEQ ID NO: 982), AUGguuggua (SEQ ID NO: 983), AUGguugguc (SEQ ID NO: 984), AUGguugguu (SEQ ID NO: 985), AUGguuguuu (SEQ ID NO: 986), AUGguuugca (SEQ ID NO: 987), AUGguuugua (SEQ ID NO: 988), AUUgcaagua (SEQ ID NO: 989), AUUguaaaua (SEQ ID NO: 990), AUUguaagau (SEQ ID NO: 991), AUUguaagca (SEQ ID NO: 992), AUUguaagga (SEQ ID NO: 993), AUUguaaggc (SEQ ID NO: 994), AUUguaagua (SEQ ID NO: 995), AUUguaaguc (SEQ ID NO: 996), AUUguaaguu (SEQ ID NO: 997), AUUguaauua (SEQ ID NO: 998), AUUguaauuu (SEQ ID NO: 999), AUUguacaaa (SEQ ID NO: 1000), AUUguaccuc (SEQ ID NO: 1001), AUUguacgug (SEQ ID NO: 1002), AUUguacuug (SEQ ID NO: 1003), AUUguaggua (SEQ ID NO: 1004), AUUguaugag (SEQ ID NO: 1005), AUUguaugua (SEQ ID NO: 1006), AUUgucuguu (SEQ ID NO: 1007), AUUgugagcu (SEQ ID NO: 1008), AUUgugagua (SEQ ID NO: 1009), AUUgugaguc (SEQ ID NO: 1010), AUUgugaguu (SEQ ID NO: 1011), AUUgugcgug (SEQ ID NO: 1012), AUUgugggug (SEQ ID NO: 1013), AUUguuagug (SEQ ID NO: 1014), CAAguaaaaa (SEQ ID NO: 1015), CAAguaaaua (SEQ ID NO: 1016), CAAguaaauc (SEQ ID NO: 1017), CAAguaaaug (SEQ ID NO: 1018), CAAguaaccc (SEQ ID NO: 1019), CAAguaacua (SEQ ID NO: 1020), CAAguaacug (SEQ ID NO: 1021), CAAguaagaa (SEQ ID NO: 1022), CAAguaagac (SEQ ID NO: 1023), CAAguaagau (SEQ ID NO: 1024), CAAguaaggu (SEQ ID NO: 1025), CAAguaagua (SEQ ID NO: 1026), CAAguaaguc (SEQ ID NO: 1027), CAAguaagug (SEQ ID NO: 1028), CAAguaaguu (SEQ ID NO: 1029), CAAguaaucc (SEQ ID NO: 1030), CAAguaaucu (SEQ ID NO: 1031), CAAguaauua (SEQ ID NO: 1032), CAAguaauuc (SEQ ID NO: 1033), CAAguaauug (SEQ ID NO: 1034), CAAguaauuu (SEQ ID NO: 1035), CAAguacaca (SEQ ID NO: 1036), CAAguacguu (SEQ ID NO: 1037), CAAguacuuu (SEQ ID NO: 1038), CAAguagcug (SEQ ID NO: 1039), CAAguaggau (SEQ ID NO: 1040), CAAguaggua (SEQ ID NO: 1041), CAAguagguc (SEQ ID NO: 1042), CAAguaggug (SEQ ID NO: 1043), CAAguagguu (SEQ ID NO: 1044), CAAguaguuu (SEQ ID NO: 1045), CAAguauaac (SEQ ID NO: 1046), CAAguauaug (SEQ ID NO: 1047), CAAguaucuu (SEQ ID NO: 1048), CAAguaugag (SEQ ID NO: 1049), CAAguaugua (SEQ ID NO: 1050), CAAguauguc (SEQ ID NO: 1051), CAAguaugug (SEQ ID NO: 1052), CAAguauguu (SEQ ID NO: 1053), CAAguauuga (SEQ ID NO: 1054), CAAguauuuc (SEQ ID NO: 1055), CAAgucagac (SEQ ID NO: 1056), CAAgucagua (SEQ ID NO: 1057), CAAgucuaua (SEQ ID NO: 1058), CAAgucugau (SEQ ID NO: 1059), CAAgugacuu (SEQ ID NO: 1060), CAAgugagaa (SEQ ID NO: 1061), CAAgugagac (SEQ ID NO: 1062), CAAgugagca (SEQ ID NO: 1063), CAAgugaggc (SEQ ID NO: 1064), CAAgugaggg (SEQ ID NO: 1065), CAAgugagua (SEQ ID NO: 1066), CAAgugaguc (SEQ ID NO: 1067), CAAgugagug (SEQ ID NO: 1068), CAAgugaucc (SEQ ID NO: 1069), CAAgugaucu (SEQ ID NO: 1070), CAAgugauuc (SEQ ID NO: 1071), CAAgugauug (SEQ ID NO: 1072), CAAgugauuu (SEQ ID NO: 1073), CAAgugccuu (SEQ ID NO: 1074), CAAgugggua (SEQ ID NO: 1075), CAAguggguc (SEQ ID NO: 1076), CAAgugggug (SEQ ID NO: 1077), CAAgugugag (SEQ ID NO: 1078), CAAguuaaaa (SEQ ID NO: 1079), CAAguuaagu (SEQ ID NO: 1080), CAAguuaauc (SEQ ID NO: 1081), CAAguuagaa (SEQ ID NO: 1082), CAAguuaguu (SEQ ID NO: 1083), CAAguucaag (SEQ ID NO: 1084), CAAguuccgu (SEQ ID NO: 1085), CAAguuggua (SEQ ID NO: 1086), CAAguuuagu (SEQ ID NO: 1087), CAAguuucca (SEQ ID NO: 1088), CAAguuuguu (SEQ ID NO: 1089), CACguaagag (SEQ ID NO: 1090), CACguaagca (SEQ ID NO: 1091), CACguaauug (SEQ ID NO: 1092), CACguaggac (SEQ ID NO: 1093), CACguaucga (SEQ ID NO: 1094), CACgucaguu (SEQ ID NO: 1095), CACgugagcu (SEQ ID NO: 1096), CACgugaguc (SEQ ID NO: 1097), CACgugagug (SEQ ID NO: 1098), CAGgcaagaa (SEQ ID NO: 1099), CAGgcaagac (SEQ ID NO: 1100), CAGgcaagag (SEQ ID NO: 1101), CAGgcaagga (SEQ ID NO: 1102), CAGgcaagua (SEQ ID NO: 1103), CAGgcaagug (SEQ ID NO: 1104), CAGgcaaguu (SEQ ID NO: 1105), CAGgcacgca (SEQ ID NO: 1106), CAGgcagagg (SEQ ID NO: 1107), CAGgcaggug (SEQ ID NO: 1108), CAGgcaucau (SEQ ID NO: 1109), CAGgcaugaa (SEQ ID NO: 1110), CAGgcaugag (SEQ ID NO: 1111), CAGgcaugca (SEQ ID NO: 1112), CAGgcaugcg (SEQ ID NO: 1113), CAGgcaugug (SEQ ID NO: 1114), CAGgcgagag (SEQ ID NO: 1115), CAGgcgccug (SEQ ID NO: 1116), CAGgcgugug (SEQ ID NO: 1117), CAGguaaaaa (SEQ ID NO: 1118), CAGguaaaag (SEQ ID NO: 1119), CAGguaaaca (SEQ ID NO: 1120), CAGguaaacc (SEQ ID NO: 1121), CAGguaaaga (SEQ ID NO: 1122), CAGguaaagc (SEQ ID NO: 1123), CAGguaaagu (SEQ ID NO: 1124), CAGguaaaua (SEQ ID NO: 1125), CAGguaaauc (SEQ ID NO: 1126), CAGguaaaug (SEQ ID NO: 1127), CAGguaaauu (SEQ ID NO: 1128), CAGguaacag (SEQ ID NO: 1129), CAGguaacau (SEQ ID NO: 1130), CAGguaacca (SEQ ID NO: 1131), CAGguaaccg (SEQ ID NO: 1132), CAGguaacgu (SEQ ID NO: 1133), CAGguaacua (SEQ ID NO: 1134), CAGguaacuc (SEQ ID NO: 1135), CAGguaacug (SEQ ID NO: 1136), CAGguaacuu (SEQ ID NO: 1137), CAGguaagaa (SEQ ID NO: 1138), CAGguaagac (SEQ ID NO: 1139), CAGguaagag (SEQ ID NO: 1140), CAGguaagau (SEQ ID NO: 1141), CAGguaagcc (SEQ ID NO: 1142), CAGguaagga (SEQ ID NO: 1143), CAGguaaggc (SEQ ID NO: 1144), CAGguaaggg (SEQ ID NO: 1145), CAGguaaggu (SEQ ID NO: 1146), CAGguaagua (SEQ ID NO: 1147), CAGguaagug (SEQ ID NO: 1148), CAGguaaguu (SEQ ID NO: 1149), CAGguaauaa (SEQ ID NO: 1150), CAGguaauau (SEQ ID NO: 1151), CAGguaaucc (SEQ ID NO: 1152), CAGguaaugc (SEQ ID NO: 1153), CAGguaaugg (SEQ ID NO: 1154), CAGguaaugu (SEQ ID NO: 1155), CAGguaauua (SEQ ID NO: 1156), CAGguaauuc (SEQ ID NO: 1157), CAGguaauug (SEQ ID NO: 1158), CAGguaauuu (SEQ ID NO: 1159), CAGguacaaa (SEQ ID NO: 1160), CAGguacaag (SEQ ID NO: 1161), CAGguacaau (SEQ ID NO: 1162), CAGguacaca (SEQ ID NO: 1163), CAGguacacg (SEQ ID NO: 1164), CAGguacaga (SEQ ID NO: 1165), CAGguacagg (SEQ ID NO: 1166), CAGguacagu (SEQ ID NO: 1167), CAGguacaua (SEQ ID NO: 1168), CAGguacaug (SEQ ID NO: 1169), CAGguacauu (SEQ ID NO: 1170), CAGguaccac (SEQ ID NO: 1171), CAGguaccca (SEQ ID NO: 1172), CAGguacccg (SEQ ID NO: 1173), CAGguacccu (SEQ ID NO: 1174), CAGguaccgc (SEQ ID NO: 1175), CAGguaccgg (SEQ ID NO: 1176), CAGguaccuc (SEQ ID NO: 1177), CAGguaccug (SEQ ID NO: 1178), CAGguaccuu (SEQ ID NO: 1179), CAGguacgag (SEQ ID NO: 1180), CAGguacgca (SEQ ID NO: 1181), CAGguacgcc (SEQ ID NO: 1182), CAGguacggu (SEQ ID NO: 1183), CAGguacgua (SEQ ID NO: 1184), CAGguacgug (SEQ ID NO: 1185), CAGguacuaa (SEQ ID NO: 1186), CAGguacuag (SEQ ID NO: 1187), CAGguacuau (SEQ ID NO: 1188), CAGguacucc (SEQ ID NO: 1189), CAGguacucu (SEQ ID NO: 1190), CAGguacuga (SEQ ID NO: 1191), CAGguacugc (SEQ ID NO: 1192), CAGguacugu (SEQ ID NO: 1193), CAGguacuua (SEQ ID NO: 1194), CAGguacuuu (SEQ ID NO: 1195), CAGguagaaa (SEQ ID NO: 1196), CAGguagaac (SEQ ID NO: 1197), CAGguagaag (SEQ ID NO: 1198), CAGguagaca (SEQ ID NO: 1199), CAGguagacc (SEQ ID NO: 1200), CAGguagaga (SEQ ID NO: 1201), CAGguagauu (SEQ ID NO: 1202), CAGguagcaa (SEQ ID NO: 1203), CAGguagcac (SEQ ID NO: 1204), CAGguagcag (SEQ ID NO: 1205), CAGguagcca (SEQ ID NO: 1206), CAGguagcgu (SEQ ID NO: 1207), CAGguagcua (SEQ ID NO: 1208), CAGguagcuc (SEQ ID NO: 1209), CAGguagcug (SEQ ID NO: 1210), CAGguagcuu (SEQ ID NO: 1211), CAGguaggaa (SEQ ID NO: 1212), CAGguaggac (SEQ ID NO: 1213), CAGguaggag (SEQ ID NO: 1214), CAGguaggca (SEQ ID NO: 1215), CAGguaggga (SEQ ID NO: 1216), CAGguagggc (SEQ ID NO: 1217), CAGguagggg (SEQ ID NO: 1218), CAGguagggu (SEQ ID NO: 1219), CAGguaggua (SEQ ID NO: 1220), CAGguagguc (SEQ ID NO: 1221), CAGguaggug (SEQ ID NO: 1222), CAGguagguu (SEQ ID NO: 1223), CAGguaguaa (SEQ ID NO: 1224), CAGguaguau (SEQ ID NO: 1225), CAGguaguca (SEQ ID NO: 1226), CAGguagucc (SEQ ID NO: 1227), CAGguaguga (SEQ ID NO: 1228), CAGguagugu (SEQ ID NO: 1229), CAGguaguuc (SEQ ID NO: 1230), CAGguaguug (SEQ ID NO: 1231), CAGguaguuu (SEQ ID NO: 1232), CAGguauaag (SEQ ID NO: 1233), CAGguauaca (SEQ ID NO: 1234), CAGguauaga (SEQ ID NO: 1235), CAGguauauc (SEQ ID NO: 1236), CAGguauaug (SEQ ID NO: 1237), CAGguauauu (SEQ ID NO: 1238), CAGguaucag (SEQ ID NO: 1239), CAGguaucau (SEQ ID NO: 1240), CAGguauccu (SEQ ID NO: 1241), CAGguaucga (SEQ ID NO: 1242), CAGguaucgc (SEQ ID NO: 1243), CAGguaucua (SEQ ID NO: 1244), CAGguaucug (SEQ ID NO: 1245), CAGguaucuu (SEQ ID NO: 1246), CAGguaugaa (SEQ ID NO: 1247), CAGguaugac (SEQ ID NO: 1248), CAGguaugag (SEQ ID NO: 1249), CAGguaugau (SEQ ID NO: 1250), CAGguaugca (SEQ ID NO: 1251), CAGguaugcc (SEQ ID NO: 1252), CAGguaugcg (SEQ ID NO: 1253), CAGguaugcu (SEQ ID NO: 1254), CAGguaugga (SEQ ID NO: 1255), CAGguauggg (SEQ ID NO: 1256), CAGguauggu (SEQ ID NO: 1257), CAGguaugua (SEQ ID NO: 1258), CAGguauguc (SEQ ID NO: 1259), CAGguaugug (SEQ ID NO: 1260), CAGguauguu (SEQ ID NO: 1261), CAGguauuau (SEQ ID NO: 1262), CAGguauuca (SEQ ID NO: 1263), CAGguauucu (SEQ ID NO: 1264), CAGguauuga (SEQ ID NO: 1265), CAGguauugg (SEQ ID NO: 1266), CAGguauugu (SEQ ID NO: 1267), CAGguauuua (SEQ ID NO: 1268), CAGguauuuc (SEQ ID NO: 1269), CAGguauuug (SEQ ID NO: 1270), CAGguauuuu (SEQ ID NO: 1271), CAGgucaaca (SEQ ID NO: 1272), CAGgucaaug (SEQ ID NO: 1273), CAGgucacgu (SEQ ID NO: 1274), CAGgucagaa (SEQ ID NO: 1275), CAGgucagac (SEQ ID NO: 1276), CAGgucagca (SEQ ID NO: 1277), CAGgucagcc (SEQ ID NO: 1278), CAGgucagcg (SEQ ID NO: 1279), CAGgucagga (SEQ ID NO: 1280), CAGgucagua (SEQ ID NO: 1281), CAGgucaguc (SEQ ID NO: 1282), CAGgucagug (SEQ ID NO: 1283), CAGgucaguu (SEQ ID NO: 1284), CAGgucaucc (SEQ ID NO: 1285), CAGgucaugc (SEQ ID NO: 1286), CAGgucauua (SEQ ID NO: 1287), CAGgucauuu (SEQ ID NO: 1288), CAGguccacc (SEQ ID NO: 1289), CAGguccacu (SEQ ID NO: 1290), CAGguccagu (SEQ ID NO: 1291), CAGguccauc (SEQ ID NO: 1292), CAGguccauu (SEQ ID NO: 1293), CAGgucccag (SEQ ID NO: 1294), CAGgucccug (SEQ ID NO: 1295), CAGguccuga (SEQ ID NO: 1296), CAGguccugc (SEQ ID NO: 1297), CAGguccugg (SEQ ID NO: 1298), CAGgucggcc (SEQ ID NO: 1299), CAGgucggug (SEQ ID NO: 1300), CAGgucguug (SEQ ID NO: 1301), CAGgucucuc (SEQ ID NO: 1302), CAGgucucuu (SEQ ID NO: 1303), CAGgucugag (SEQ ID NO: 1304), CAGgucugcc (SEQ ID NO: 1305), CAGgucugcg (SEQ ID NO: 1306), CAGgucugga (SEQ ID NO: 1307), CAGgucuggu (SEQ ID NO: 1308), CAGgucugua (SEQ ID NO: 1309), CAGgucuguc (SEQ ID NO: 1310), CAGgucugug (SEQ ID NO: 1311), CAGgucuguu (SEQ ID NO: 1312), CAGgucuucc (SEQ ID NO: 1313), CAGgucuuuc (SEQ ID NO: 1314), CAGgugaaag (SEQ ID NO: 1315), CAGgugaaau (SEQ ID NO: 1316), CAGgugaaca (SEQ ID NO: 1317), CAGgugaaga (SEQ ID NO: 1318), CAGgugaagg (SEQ ID NO: 1319), CAGgugaaua (SEQ ID NO: 1320), CAGgugaauc (SEQ ID NO: 1321), CAGgugaauu (SEQ ID NO: 1322), CAGgugacaa (SEQ ID NO: 1323), CAGgugacau (SEQ ID NO: 1324), CAGgugacca (SEQ ID NO: 1325), CAGgugaccc (SEQ ID NO: 1326), CAGgugaccg (SEQ ID NO: 1327), CAGgugaccu (SEQ ID NO: 1328), CAGgugacgg (SEQ ID NO: 1329), CAGgugacua (SEQ ID NO: 1330), CAGgugacuc (SEQ ID NO: 1331), CAGgugacug (SEQ ID NO: 1332), CAGgugagaa (SEQ ID NO: 1333), CAGgugagac (SEQ ID NO: 1334), CAGgugagag (SEQ ID NO: 1335), CAGgugagau (SEQ ID NO: 1336), CAGgugagca (SEQ ID NO: 1337), CAGgugagcc (SEQ ID NO: 1338), CAGgugagcg (SEQ ID NO: 1339), CAGgugagcu (SEQ ID NO: 1340), CAGgugagga (SEQ ID NO: 1341), CAGgugaggc (SEQ ID NO: 1342), CAGgugaggg (SEQ ID NO: 1343), CAGgugaggu (SEQ ID NO: 1344), CAGgugagua (SEQ ID NO: 1345), CAGgugaguc (SEQ ID NO: 1346), CAGgugagug (SEQ ID NO: 1347), CAGgugaguu (SEQ ID NO: 1348), CAGgugauaa (SEQ ID NO: 1349), CAGgugaucc (SEQ ID NO: 1350), CAGgugaucu (SEQ ID NO: 1351), CAGgugaugc (SEQ ID NO: 1352), CAGgugaugg (SEQ ID NO: 1353), CAGgugaugu (SEQ ID NO: 1354), CAGgugauua (SEQ ID NO: 1355), CAGgugauuc (SEQ ID NO: 1356), CAGgugauug (SEQ ID NO: 1357), CAGgugauuu (SEQ ID NO: 1358), CAGgugcaaa (SEQ ID NO: 1359), CAGgugcaag (SEQ ID NO: 1360), CAGgugcaca (SEQ ID NO: 1361), CAGgugcacg (SEQ ID NO: 1362), CAGgugcaga (SEQ ID NO: 1363), CAGgugcagg (SEQ ID NO: 1364), CAGgugcaua (SEQ ID NO: 1365), CAGgugcauc (SEQ ID NO: 1366), CAGgugcaug (SEQ ID NO: 1367), CAGgugccaa (SEQ ID NO: 1368), CAGgugccca (SEQ ID NO: 1369), CAGgugcccc (SEQ ID NO: 1370), CAGgugcccg (SEQ ID NO: 1371), CAGgugccua (SEQ ID NO: 1372), CAGgugccug (SEQ ID NO: 1373), CAGgugcgaa (SEQ ID NO: 1374), CAGgugcgca (SEQ ID NO: 1375), CAGgugcgcc (SEQ ID NO: 1376), CAGgugcgcg (SEQ ID NO: 1377), CAGgugcgga (SEQ ID NO: 1378), CAGgugcggu (SEQ ID NO: 1379), CAGgugcgua (SEQ ID NO: 1380), CAGgugcguc (SEQ ID NO: 1381), CAGgugcgug (SEQ ID NO: 1382), CAGgugcuag (SEQ ID NO: 1383), CAGgugcuau (SEQ ID NO: 1384), CAGgugcuca (SEQ ID NO: 1385), CAGgugcucc (SEQ ID NO: 1386), CAGgugcucg (SEQ ID NO: 1387), CAGgugcugc (SEQ ID NO: 1388), CAGgugcugg (SEQ ID NO: 1389), CAGgugcuua (SEQ ID NO: 1390), CAGgugcuuc (SEQ ID NO: 1391), CAGgugcuug (SEQ ID NO: 1392), CAGguggaac (SEQ ID NO: 1393), CAGguggaag (SEQ ID NO: 1394), CAGguggaau (SEQ ID NO: 1395), CAGguggaga (SEQ ID NO: 1396), CAGguggagu (SEQ ID NO: 1397), CAGguggauu (SEQ ID NO: 1398), CAGguggcca (SEQ ID NO: 1399), CAGguggcuc (SEQ ID NO: 1400), CAGguggcug (SEQ ID NO: 1401), CAGgugggaa (SEQ ID NO: 1402), CAGgugggac (SEQ ID NO: 1403), CAGgugggag (SEQ ID NO: 1404), CAGgugggau (SEQ ID NO: 1405), CAGgugggca (SEQ ID NO: 1406), CAGgugggcc (SEQ ID NO: 1407), CAGgugggcu (SEQ ID NO: 1408), CAGgugggga (SEQ ID NO: 1409), CAGguggggc (SEQ ID NO: 1410), CAGguggggg (SEQ ID NO: 1411), CAGguggggu (SEQ ID NO: 1412), CAGgugggua (SEQ ID NO: 1413), CAGguggguc (SEQ ID NO: 1414), CAGgugggug (SEQ ID NO: 1415), CAGguggguu (SEQ ID NO: 1416), CAGguggucu (SEQ ID NO: 1417), CAGguggugg (SEQ ID NO: 1418), CAGgugguug (SEQ ID NO: 1419), CAGguguaca (SEQ ID NO: 1420), CAGguguagg (SEQ ID NO: 1421), CAGguguauc (SEQ ID NO: 1422), CAGgugucac (SEQ ID NO: 1423), CAGgugucag (SEQ ID NO: 1424), CAGgugucca (SEQ ID NO: 1425), CAGguguccu (SEQ ID NO: 1426), CAGgugucua (SEQ ID NO: 1427), CAGgugucuc (SEQ ID NO: 1428), CAGgugucug (SEQ ID NO: 1429), CAGgugugaa (SEQ ID NO: 1430), CAGgugugac (SEQ ID NO: 1431), CAGgugugag (SEQ ID NO: 1432), CAGgugugau (SEQ ID NO: 1433), CAGgugugca (SEQ ID NO: 1434), CAGgugugcc (SEQ ID NO: 1435), CAGgugugcg (SEQ ID NO: 1436), CAGgugugcu (SEQ ID NO: 1437), CAGgugugga (SEQ ID NO: 1438), CAGguguggc (SEQ ID NO: 1439), CAGgugugua (SEQ ID NO: 1440), CAGguguguc (SEQ ID NO: 1441), CAGgugugug (SEQ ID NO: 1442), CAGguguguu (SEQ ID NO: 1443), CAGguguuua (SEQ ID NO: 1444), CAGguuaaaa (SEQ ID NO: 1445), CAGguuaaua (SEQ ID NO: 1446), CAGguuaauc (SEQ ID NO: 1447), CAGguuaccu (SEQ ID NO: 1448), CAGguuagaa (SEQ ID NO: 1449), CAGguuagag (SEQ ID NO: 1450), CAGguuagau (SEQ ID NO: 1451), CAGguuagcc (SEQ ID NO: 1452), CAGguuaggg (SEQ ID NO: 1453), CAGguuaggu (SEQ ID NO: 1454), CAGguuagua (SEQ ID NO: 1455), CAGguuaguc (SEQ ID NO: 1456), CAGguuagug (SEQ ID NO: 1457), CAGguuaguu (SEQ ID NO: 1458), CAGguuauca (SEQ ID NO: 1459), CAGguuaugu (SEQ ID NO: 1460), CAGguuauua (SEQ ID NO: 1461), CAGguuauug (SEQ ID NO: 1462), CAGguucaaa (SEQ ID NO: 1463), CAGguucaac (SEQ ID NO: 1464), CAGguucaag (SEQ ID NO: 1465), CAGguucaca (SEQ ID NO: 1466), CAGguucacg (SEQ ID NO: 1467), CAGguucagg (SEQ ID NO: 1468), CAGguucaug (SEQ ID NO: 1469), CAGguuccag (SEQ ID NO: 1470), CAGguuccca (SEQ ID NO: 1471), CAGguucccg (SEQ ID NO: 1472), CAGguucgaa (SEQ ID NO: 1473), CAGguucgag (SEQ ID NO: 1474), CAGguucuau (SEQ ID NO: 1475), CAGguucugc (SEQ ID NO: 1476), CAGguucuua (SEQ ID NO: 1477), CAGguucuuc (SEQ ID NO: 1478), CAGguucuuu (SEQ ID NO: 1479), CAGguugaac (SEQ ID NO: 1480), CAGguugaag (SEQ ID NO: 1481), CAGguugagu (SEQ ID NO: 1482), CAGguugaua (SEQ ID NO: 1483), CAGguuggag (SEQ ID NO: 1484), CAGguuggca (SEQ ID NO: 1485), CAGguuggcc (SEQ ID NO: 1486), CAGguugguc (SEQ ID NO: 1487), CAGguuggug (SEQ ID NO: 1488), CAGguugguu (SEQ ID NO: 1489), CAGguuguaa (SEQ ID NO: 1490), CAGguuguac (SEQ ID NO: 1491), CAGguuguau (SEQ ID NO: 1492), CAGguuguca (SEQ ID NO: 1493), CAGguuguga (SEQ ID NO: 1494), CAGguuguug (SEQ ID NO: 1495), CAGguuuaag (SEQ ID NO: 1496), CAGguuuacc (SEQ ID NO: 1497), CAGguuuagc (SEQ ID NO: 1498), CAGguuuagu (SEQ ID NO: 1499), CAGguuucuu (SEQ ID NO: 1500), CAGguuugaa (SEQ ID NO: 1501), CAGguuugag (SEQ ID NO: 1502), CAGguuugau (SEQ ID NO: 1503), CAGguuugcc (SEQ ID NO: 1504), CAGguuugcu (SEQ ID NO: 1505), CAGguuuggg (SEQ ID NO: 1506), CAGguuuggu (SEQ ID NO: 1507), CAGguuugua (SEQ ID NO: 1508), CAGguuugug (SEQ ID NO: 1509), CAGguuuguu (SEQ ID NO: 1510), CAGguuuucu (SEQ ID NO: 1511), CAGguuuugg (SEQ ID NO: 1512), CAGguuuuuc (SEQ ID NO: 1513), CAGguuuuuu (SEQ ID NO: 1514), CAUgcagguu (SEQ ID NO: 1515), CAUguaaaac (SEQ ID NO: 1516), CAUguaacua (SEQ ID NO: 1517), CAUguaagaa (SEQ ID NO: 1518), CAUguaagag (SEQ ID NO: 1519), CAUguaagau (SEQ ID NO: 1520), CAUguaagcc (SEQ ID NO: 1521), CAUguaagua (SEQ ID NO: 1522), CAUguaagug (SEQ ID NO: 1523), CAUguaaguu (SEQ ID NO: 1524), CAUguaauua (SEQ ID NO: 1525), CAUguacaua (SEQ ID NO: 1526), CAUguaccac (SEQ ID NO: 1527), CAUguacguu (SEQ ID NO: 1528), CAUguaggua (SEQ ID NO: 1529), CAUguaggug (SEQ ID NO: 1530), CAUguagguu (SEQ ID NO: 1531), CAUguaugaa (SEQ ID NO: 1532), CAUguaugua (SEQ ID NO: 1533), CAUguaugug (SEQ ID NO: 1534), CAUguauguu (SEQ ID NO: 1535), CAUgugagaa (SEQ ID NO: 1536), CAUgugagca (SEQ ID NO: 1537), CAUgugagcu (SEQ ID NO: 1538), CAUgugagua (SEQ ID NO: 1539), CAUgugaguc (SEQ ID NO: 1540), CAUgugagug (SEQ ID NO: 1541), CAUgugaguu (SEQ ID NO: 1542), CAUgugcgua (SEQ ID NO: 1543), CAUgugggaa (SEQ ID NO: 1544), CAUguggguu (SEQ ID NO: 1545), CAUgugugug (SEQ ID NO: 1546), CAUguguguu (SEQ ID NO: 1547), CAUguuaaua (SEQ ID NO: 1548), CAUguuagcc (SEQ ID NO: 1549), CCAguaagau (SEQ ID NO: 1550), CCAguaagca (SEQ ID NO: 1551), CCAguaagcc (SEQ ID NO: 1552), CCAguaagcu (SEQ ID NO: 1553), CCAguaagga (SEQ ID NO: 1554), CCAguaagua (SEQ ID NO: 1555), CCAguaaguc (SEQ ID NO: 1556), CCAguaagug (SEQ ID NO: 1557), CCAguaaguu (SEQ ID NO: 1558), CCAguaauug (SEQ ID NO: 1559), CCAguacggg (SEQ ID NO: 1560), CCAguagguc (SEQ ID NO: 1561), CCAguauugu (SEQ ID NO: 1562), CCAgugaggc (SEQ ID NO: 1563), CCAgugagua (SEQ ID NO: 1564), CCAgugagug (SEQ ID NO: 1565), CCAguggguc (SEQ ID NO: 1566), CCAguuaguu (SEQ ID NO: 1567), CCAguugagu (SEQ ID NO: 1568), CCCguaagau (SEQ ID NO: 1569), CCCguauguc (SEQ ID NO: 1570), CCCguauguu (SEQ ID NO: 1571), CCCguccugc (SEQ ID NO: 1572), CCCgugagug (SEQ ID NO: 1573), CCGguaaaga (SEQ ID NO: 1574), CCGguaagau (SEQ ID NO: 1575), CCGguaagcc (SEQ ID NO: 1576), CCGguaagga (SEQ ID NO: 1577), CCGguaaggc (SEQ ID NO: 1578), CCGguaaugg (SEQ ID NO: 1579), CCGguacagu (SEQ ID NO: 1580), CCGguacuga (SEQ ID NO: 1581), CCGguauucc (SEQ ID NO: 1582), CCGgucagug (SEQ ID NO: 1583), CCGgugaaaa (SEQ ID NO: 1584), CCGgugagaa (SEQ ID NO: 1585), CCGgugaggg (SEQ ID NO: 1586), CCGgugagug (SEQ ID NO: 1587), CCGgugaguu (SEQ ID NO: 1588), CCGgugcgcg (SEQ ID NO: 1589), CCGgugggcg (SEQ ID NO: 1590), CCGguugguc (SEQ ID NO: 1591), CCUguaaaug (SEQ ID NO: 1592), CCUguaaauu (SEQ ID NO: 1593), CCUguaagaa (SEQ ID NO: 1594), CCUguaagac (SEQ ID NO: 1595), CCUguaagag (SEQ ID NO: 1596), CCUguaagca (SEQ ID NO: 1597), CCUguaagcg (SEQ ID NO: 1598), CCUguaagga (SEQ ID NO: 1599), CCUguaaguu (SEQ ID NO: 1600), CCUguaggua (SEQ ID NO: 1601), CCUguaggug (SEQ ID NO: 1602), CCUguaucuu (SEQ ID NO: 1603), CCUguauggu (SEQ ID NO: 1604), CCUguaugug (SEQ ID NO: 1605), CCUgugagaa (SEQ ID NO: 1606), CCUgugagca (SEQ ID NO: 1607), CCUgugaggg (SEQ ID NO: 1608), CCUgugaguc (SEQ ID NO: 1609), CCUgugagug (SEQ ID NO: 1610), CCUgugaguu (SEQ ID NO: 1611), CCUguggcuc (SEQ ID NO: 1612), CCUgugggua (SEQ ID NO: 1613), CCUgugugua (SEQ ID NO: 1614), CCUguuagaa (SEQ ID NO: 1615), CGAguaaggg (SEQ ID NO: 1616), CGAguaaggu (SEQ ID NO: 1617), CGAguagcug (SEQ ID NO: 1618), CGAguaggug (SEQ ID NO: 1619), CGAguagguu (SEQ ID NO: 1620), CGAgugagca (SEQ ID NO: 1621), CGCguaagag (SEQ ID NO: 1622), CGGgcaggca (SEQ ID NO: 1623), CGGguaagcc (SEQ ID NO: 1624), CGGguaagcu (SEQ ID NO: 1625), CGGguaaguu (SEQ ID NO: 1626), CGGguaauuc (SEQ ID NO: 1627), CGGguaauuu (SEQ ID NO: 1628), CGGguacagu (SEQ ID NO: 1629), CGGguacggg (SEQ ID NO: 1630), CGGguaggag (SEQ ID NO: 1631), CGGguaggcc (SEQ ID NO: 1632), CGGguaggug (SEQ ID NO: 1633), CGGguauuua (SEQ ID NO: 1634), CGGgucugag (SEQ ID NO: 1635), CGGgugaccg (SEQ ID NO: 1636), CGGgugacuc (SEQ ID NO: 1637), CGGgugagaa (SEQ ID NO: 1638), CGGgugaggg (SEQ ID NO: 1639), CGGgugaggu (SEQ ID NO: 1640), CGGgugagua (SEQ ID NO: 1641), CGGgugagug (SEQ ID NO: 1642), CGGgugaguu (SEQ ID NO: 1643), CGGgugauuu (SEQ ID NO: 1644), CGGgugccuu (SEQ ID NO: 1645), CGGgugggag (SEQ ID NO: 1646), CGGgugggug (SEQ ID NO: 1647), CGGguggguu (SEQ ID NO: 1648), CGGguguguc (SEQ ID NO: 1649), CGGgugugug (SEQ ID NO: 1650), CGGguguguu (SEQ ID NO: 1651), CGGguucaag (SEQ ID NO: 1652), CGGguucaug (SEQ ID NO: 1653), CGGguuugcu (SEQ ID NO: 1654), CGUguagggu (SEQ ID NO: 1655), CGUguaugca (SEQ ID NO: 1656), CGUguaugua (SEQ ID NO: 1657), CGUgucugua (SEQ ID NO: 1658), CGUgugagug (SEQ ID NO: 1659), CGUguuuucu (SEQ ID NO: 1660), CUAguaaaug (SEQ ID NO: 1661), CUAguaagcg (SEQ ID NO: 1662), CUAguaagcu (SEQ ID NO: 1663), CUAguaagua (SEQ ID NO: 1664), CUAguaaguc (SEQ ID NO: 1665), CUAguaagug (SEQ ID NO: 1666), CUAguaaguu (SEQ ID NO: 1667), CUAguaauuu (SEQ ID NO: 1668), CUAguaggua (SEQ ID NO: 1669), CUAguagguu (SEQ ID NO: 1670), CUAguaugua (SEQ ID NO: 1671), CUAguauguu (SEQ ID NO: 1672), CUAgugagua (SEQ ID NO: 1673), CUCguaagca (SEQ ID NO: 1674), CUCguaagug (SEQ ID NO: 1675), CUCguaaguu (SEQ ID NO: 1676), CUCguaucug (SEQ ID NO: 1677), CUCgucugug (SEQ ID NO: 1678), CUCgugaaua (SEQ ID NO: 1679), CUCgugagua (SEQ ID NO: 1680), CUCgugauua (SEQ ID NO: 1681), CUGguaaaaa (SEQ ID NO: 1682), CUGguaaaau (SEQ ID NO: 1683), CUGguaaacc (SEQ ID NO: 1684), CUGguaaacg (SEQ ID NO: 1685), CUGguaaagc (SEQ ID NO: 1686), CUGguaaaua (SEQ ID NO: 1687), CUGguaaauc (SEQ ID NO: 1688), CUGguaaaug (SEQ ID NO: 1689), CUGguaaauu (SEQ ID NO: 1690), CUGguaacac (SEQ ID NO: 1691), CUGguaacag (SEQ ID NO: 1692), CUGguaaccc (SEQ ID NO: 1693), CUGguaaccg (SEQ ID NO: 1694), CUGguaacug (SEQ ID NO: 1695), CUGguaacuu (SEQ ID NO: 1696), CUGguaagaa (SEQ ID NO: 1697), CUGguaagag (SEQ ID NO: 1698), CUGguaagau (SEQ ID NO: 1699), CUGguaagca (SEQ ID NO: 1700), CUGguaagcc (SEQ ID NO: 1701), CUGguaagcu (SEQ ID NO: 1702), CUGguaagga (SEQ ID NO: 1703), CUGguaaggc (SEQ ID NO: 1704), CUGguaaggg (SEQ ID NO: 1705), CUGguaaggu (SEQ ID NO: 1706), CUGguaagua (SEQ ID NO: 1707), CUGguaagug (SEQ ID NO: 1708), CUGguaaguu (SEQ ID NO: 1709), CUGguaauga (SEQ ID NO: 1710), CUGguaaugc (SEQ ID NO: 1711), CUGguaauuc (SEQ ID NO: 1712), CUGguaauuu (SEQ ID NO: 1713), CUGguacaac (SEQ ID NO: 1714), CUGguacaau (SEQ ID NO: 1715), CUGguacaga (SEQ ID NO: 1716), CUGguacaua (SEQ ID NO: 1717), CUGguacauu (SEQ ID NO: 1718), CUGguaccau (SEQ ID NO: 1719), CUGguacguu (SEQ ID NO: 1720), CUGguacuaa (SEQ ID NO: 1721), CUGguacuug (SEQ ID NO: 1722), CUGguacuuu (SEQ ID NO: 1723), CUGguagaga (SEQ ID NO: 1724), CUGguagaua (SEQ ID NO: 1725), CUGguagcgu (SEQ ID NO: 1726), CUGguaggau (SEQ ID NO: 1727), CUGguaggca (SEQ ID NO: 1728), CUGguaggua (SEQ ID NO: 1729), CUGguagguc (SEQ ID NO: 1730), CUGguaggug (SEQ ID NO: 1731), CUGguaucaa (SEQ ID NO: 1732), CUGguaugau (SEQ ID NO: 1733), CUGguauggc (SEQ ID NO: 1734), CUGguauggu (SEQ ID NO: 1735), CUGguaugua (SEQ ID NO: 1736), CUGguaugug (SEQ ID NO: 1737), CUGguauguu (SEQ ID NO: 1738), CUGguauuga (SEQ ID NO: 1739), CUGguauuuc (SEQ ID NO: 1740), CUGguauuuu (SEQ ID NO: 1741), CUGgucaaca (SEQ ID NO: 1742), CUGgucagag (SEQ ID NO: 1743), CUGgucccgc (SEQ ID NO: 1744), CUGgucggua (SEQ ID NO: 1745), CUGgucuggg (SEQ ID NO: 1746), CUGgugaagu (SEQ ID NO: 1747), CUGgugaaua (SEQ ID NO: 1748), CUGgugaauu (SEQ ID NO: 1749), CUGgugacua (SEQ ID NO: 1750), CUGgugagaa (SEQ ID NO: 1751), CUGgugagac (SEQ ID NO: 1752), CUGgugagca (SEQ ID NO: 1753), CUGgugagcu (SEQ ID NO: 1754), CUGgugagga (SEQ ID NO: 1755), CUGgugaggc (SEQ ID NO: 1756), CUGgugaggg (SEQ ID NO: 1757), CUGgugaggu (SEQ ID NO: 1758), CUGgugagua (SEQ ID NO: 1759), CUGgugaguc (SEQ ID NO: 1760), CUGgugagug (SEQ ID NO: 1761), CUGgugaguu (SEQ ID NO: 1762), CUGgugauua (SEQ ID NO: 1763), CUGgugauuu (SEQ ID NO: 1764), CUGgugcaga (SEQ ID NO: 1765), CUGgugcgcu (SEQ ID NO: 1766), CUGgugcgug (SEQ ID NO: 1767), CUGgugcuga (SEQ ID NO: 1768), CUGgugggag (SEQ ID NO: 1769), CUGgugggga (SEQ ID NO: 1770), CUGgugggua (SEQ ID NO: 1771), CUGguggguc (SEQ ID NO: 1772), CUGgugggug (SEQ ID NO: 1773), CUGguggguu (SEQ ID NO: 1774), CUGgugugaa (SEQ ID NO: 1775), CUGgugugca (SEQ ID NO: 1776), CUGgugugcu (SEQ ID NO: 1777), CUGguguggu (SEQ ID NO: 1778), CUGgugugug (SEQ ID NO: 1779), CUGguguguu (SEQ ID NO: 1780), CUGguuagcu (SEQ ID NO: 1781), CUGguuagug (SEQ ID NO: 1782), CUGguucgug (SEQ ID NO: 1783), CUGguuggcu (SEQ ID NO: 1784), CUGguuguuu (SEQ ID NO: 1785), CUGguuugua (SEQ ID NO: 1786), CUGguuuguc (SEQ ID NO: 1787), CUGguuugug (SEQ ID NO: 1788), CUUguaaaug (SEQ ID NO: 1789), CUUguaagcu (SEQ ID NO: 1790), CUUguaagga (SEQ ID NO: 1791), CUUguaaggc (SEQ ID NO: 1792), CUUguaagua (SEQ ID NO: 1793), CUUguaagug (SEQ ID NO: 1794), CUUguaaguu (SEQ ID NO: 1795), CUUguacguc (SEQ ID NO: 1796), CUUguacgug (SEQ ID NO: 1797), CUUguaggua (SEQ ID NO: 1798), CUUguagugc (SEQ ID NO: 1799), CUUguauagg (SEQ ID NO: 1800), CUUgucagua (SEQ ID NO: 1801), CUUgugagua (SEQ ID NO: 1802), CUUgugaguc (SEQ ID NO: 1803), CUUgugaguu (SEQ ID NO: 1804), CUUguggguu (SEQ ID NO: 1805), CUUgugugua (SEQ ID NO: 1806), CUUguuagug (SEQ ID NO: 1807), CUUguuugag (SEQ ID NO: 1808), GAAguaaaac (SEQ ID NO: 1809), GAAguaaagc (SEQ ID NO: 1810), GAAguaaagu (SEQ ID NO: 1811), GAAguaaaua (SEQ ID NO: 1812), GAAguaaauu (SEQ ID NO: 1813), GAAguaagaa (SEQ ID NO: 1814), GAAguaagcc (SEQ ID NO: 1815), GAAguaagcu (SEQ ID NO: 1816), GAAguaagga (SEQ ID NO: 1817), GAAguaagua (SEQ ID NO: 1818), GAAguaagug (SEQ ID NO: 1819), GAAguaaguu (SEQ ID NO: 1820), GAAguaauau (SEQ ID NO: 1821), GAAguaaugc (SEQ ID NO: 1822), GAAguaauua (SEQ ID NO: 1823), GAAguaauuu (SEQ ID NO: 1824), GAAguaccau (SEQ ID NO: 1825), GAAguacgua (SEQ ID NO: 1826), GAAguacguc (SEQ ID NO: 1827), GAAguaggca (SEQ ID NO: 1828), GAAguagguc (SEQ ID NO: 1829), GAAguauaaa (SEQ ID NO: 1830), GAAguaugcu (SEQ ID NO: 1831), GAAguaugug (SEQ ID NO: 1832), GAAguauguu (SEQ ID NO: 1833), GAAguauuaa (SEQ ID NO: 1834), GAAgucagug (SEQ ID NO: 1835), GAAgugagag (SEQ ID NO: 1836), GAAgugagcg (SEQ ID NO: 1837), GAAgugaggu (SEQ ID NO: 1838), GAAgugaguc (SEQ ID NO: 1839), GAAgugagug (SEQ ID NO: 1840), GAAgugaguu (SEQ ID NO: 1841), GAAgugauaa (SEQ ID NO: 1842), GAAgugauuc (SEQ ID NO: 1843), GAAgugcgug (SEQ ID NO: 1844), GAAguguggg (SEQ ID NO: 1845), GAAguguguc (SEQ ID NO: 1846), GAAguuggug (SEQ ID NO: 1847), GACguaaagu (SEQ ID NO: 1848), GACguaagcu (SEQ ID NO: 1849), GACguaagua (SEQ ID NO: 1850), GACguaaugg (SEQ ID NO: 1851), GACguaugcc (SEQ ID NO: 1852), GACguauguu (SEQ ID NO: 1853), GACgugagcc (SEQ ID NO: 1854), GACgugagug (SEQ ID NO: 1855), GAGgcaaaug (SEQ ID NO: 1856), GAGgcaagag (SEQ ID NO: 1857), GAGgcaagua (SEQ ID NO: 1858), GAGgcaagug (SEQ ID NO: 1859), GAGgcaaguu (SEQ ID NO: 1860), GAGgcacgag (SEQ ID NO: 1861), GAGgcaggga (SEQ ID NO: 1862), GAGgcaugug (SEQ ID NO: 1863), GAGgcgaagg (SEQ ID NO: 1864), GAGguaaaaa (SEQ ID NO: 1865), GAGguaaaac (SEQ ID NO: 1866), GAGguaaaag (SEQ ID NO: 1867), GAGguaaaau (SEQ ID NO: 1868), GAGguaaacc (SEQ ID NO: 1869), GAGguaaaga (SEQ ID NO: 1870), GAGguaaagc (SEQ ID NO: 1871), GAGguaaagu (SEQ ID NO: 1872), GAGguaaaua (SEQ ID NO: 1873), GAGguaaauc (SEQ ID NO: 1874), GAGguaaaug (SEQ ID NO: 1875), GAGguaaauu (SEQ ID NO: 1876), GAGguaacaa (SEQ ID NO: 1877), GAGguaacag (SEQ ID NO: 1878), GAGguaacca (SEQ ID NO: 1879), GAGguaaccu (SEQ ID NO: 1880), GAGguaacuu (SEQ ID NO: 1881), GAGguaagaa (SEQ ID NO: 1882), GAGguaagag (SEQ ID NO: 1883), GAGguaagau (SEQ ID NO: 1884), GAGguaagca (SEQ ID NO: 1885), GAGguaagcc (SEQ ID NO: 1886), GAGguaagcg (SEQ ID NO: 1887), GAGguaagcu (SEQ ID NO: 1888), GAGguaagga (SEQ ID NO: 1889), GAGguaaggc (SEQ ID NO: 1890), GAGguaaggg (SEQ ID NO: 1891), GAGguaaggu (SEQ ID NO: 1892), GAGguaagua (SEQ ID NO: 1893), GAGguaaguc (SEQ ID NO: 1894), GAGguaauaa (SEQ ID NO: 1895), GAGguaauac (SEQ ID NO: 1896), GAGguaauau (SEQ ID NO: 1897), GAGguaauca (SEQ ID NO: 1898), GAGguaaucu (SEQ ID NO: 1899), GAGguaaugg (SEQ ID NO: 1900), GAGguaaugu (SEQ ID NO: 1901), GAGguaauug (SEQ ID NO: 1902), GAGguaauuu (SEQ ID NO: 1903), GAGguacaaa (SEQ ID NO: 1904), GAGguacaac (SEQ ID NO: 1905), GAGguacaga (SEQ ID NO: 1906), GAGguacagc (SEQ ID NO: 1907), GAGguacagu (SEQ ID NO: 1908), GAGguacaua (SEQ ID NO: 1909), GAGguacauu (SEQ ID NO: 1910), GAGguaccag (SEQ ID NO: 1911), GAGguaccga (SEQ ID NO: 1912), GAGguaccug (SEQ ID NO: 1913), GAGguaccuu (SEQ ID NO: 1914), GAGguacuag (SEQ ID NO: 1915), GAGguacuau (SEQ ID NO: 1916), GAGguacucc (SEQ ID NO: 1917), GAGguacugc (SEQ ID NO: 1918), GAGguacugg (SEQ ID NO: 1919), GAGguacugu (SEQ ID NO: 1920), GAGguacuug (SEQ ID NO: 1921), GAGguacuuu (SEQ ID NO: 1922), GAGguagaag (SEQ ID NO: 1923), GAGguagaga (SEQ ID NO: 1924), GAGguagagg (SEQ ID NO: 1925), GAGguagagu (SEQ ID NO: 1926), GAGguagauc (SEQ ID NO: 1927), GAGguagcua (SEQ ID NO: 1928), GAGguagcug (SEQ ID NO: 1929), GAGguaggaa (SEQ ID NO: 1930), GAGguaggag (SEQ ID NO: 1931), GAGguaggca (SEQ ID NO: 1932), GAGguaggcu (SEQ ID NO: 1933), GAGguaggga (SEQ ID NO: 1934), GAGguagggc (SEQ ID NO: 1935), GAGguagggg (SEQ ID NO: 1936), GAGguaggua (SEQ ID NO: 1937), GAGguaggug (SEQ ID NO: 1938), GAGguagguu (SEQ ID NO: 1939), GAGguaguaa (SEQ ID NO: 1940), GAGguaguag (SEQ ID NO: 1941), GAGguaguau (SEQ ID NO: 1942), GAGguagucu (SEQ ID NO: 1943), GAGguagugc (SEQ ID NO: 1944), GAGguagugg (SEQ ID NO: 1945), GAGguaguua (SEQ ID NO: 1946), GAGguaguug (SEQ ID NO: 1947), GAGguauaag (SEQ ID NO: 1948), GAGguauacu (SEQ ID NO: 1949), GAGguauagc (SEQ ID NO: 1950), GAGguauaug (SEQ ID NO: 1951), GAGguauauu (SEQ ID NO: 1952), GAGguaucau (SEQ ID NO: 1953), GAGguaucug (SEQ ID NO: 1954), GAGguaucuu (SEQ ID NO: 1955), GAGguaugaa (SEQ ID NO: 1956), GAGguaugac (SEQ ID NO: 1957), GAGguaugag (SEQ ID NO: 1958), GAGguaugcc (SEQ ID NO: 1959), GAGguaugcg (SEQ ID NO: 1960), GAGguaugcu (SEQ ID NO: 1961), GAGguaugga (SEQ ID NO: 1962), GAGguauggg (SEQ ID NO: 1963), GAGguauggu (SEQ ID NO: 1964), GAGguaugua (SEQ ID NO: 1965), GAGguauguc (SEQ ID NO: 1966), GAGguaugug (SEQ ID NO: 1967), GAGguauguu (SEQ ID NO: 1968), GAGguauucc (SEQ ID NO: 1969), GAGguauuga (SEQ ID NO: 1970), GAGguauugu (SEQ ID NO: 1971), GAGguauuua (SEQ ID NO: 1972), GAGguauuuc (SEQ ID NO: 1973), GAGguauuug (SEQ ID NO: 1974), GAGguauuuu (SEQ ID NO: 1975), GAGgucaaca (SEQ ID NO: 1976), GAGgucaagg (SEQ ID NO: 1977), GAGgucaaug (SEQ ID NO: 1978), GAGgucacug (SEQ ID NO: 1979), GAGgucagaa (SEQ ID NO: 1980), GAGgucagag (SEQ ID NO: 1981), GAGgucagcu (SEQ ID NO: 1982), GAGgucagga (SEQ ID NO: 1983), GAGgucaggc (SEQ ID NO: 1984), GAGgucaggg (SEQ ID NO: 1985), GAGgucaggu (SEQ ID NO: 1986), GAGgucagua (SEQ ID NO: 1987), GAGgucauau (SEQ ID NO: 1988), GAGgucaugu (SEQ ID NO: 1989), GAGgucauuu (SEQ ID NO: 1990), GAGguccaua (SEQ ID NO: 1991), GAGguccauc (SEQ ID NO: 1992), GAGguccggg (SEQ ID NO: 1993), GAGguccggu (SEQ ID NO: 1994), GAGguccuug (SEQ ID NO: 1995), GAGgucgggg (SEQ ID NO: 1996), GAGgucucgu (SEQ ID NO: 1997), GAGgucugag (SEQ ID NO: 1998), GAGgucuggu (SEQ ID NO: 1999), GAGgucuguc (SEQ ID NO: 2000), GAGgucuguu (SEQ ID NO: 2001), GAGgucuuuu (SEQ ID NO: 2002), GAGgugaaaa (SEQ ID NO: 2003), GAGgugaaau (SEQ ID NO: 2004), GAGgugaaca (SEQ ID NO: 2005), GAGgugaagg (SEQ ID NO: 2006), GAGgugaaua (SEQ ID NO: 2007), GAGgugaauu (SEQ ID NO: 2008), GAGgugacau (SEQ ID NO: 2009), GAGgugacca (SEQ ID NO: 2010), GAGgugaccu (SEQ ID NO: 2011), GAGgugacua (SEQ ID NO: 2012), GAGgugacuu (SEQ ID NO: 2013), GAGgugagaa (SEQ ID NO: 2014), GAGgugagac (SEQ ID NO: 2015), GAGgugagag (SEQ ID NO: 2016), GAGgugagau (SEQ ID NO: 2017), GAGgugagca (SEQ ID NO: 2018), GAGgugagcc (SEQ ID NO: 2019), GAGgugagcg (SEQ ID NO: 2020), GAGgugagcu (SEQ ID NO: 2021), GAGgugagga (SEQ ID NO: 2022), GAGgugaggc (SEQ ID NO: 2023), GAGgugaggg (SEQ ID NO: 2024), GAGgugagua (SEQ ID NO: 2025), GAGgugagug (SEQ ID NO: 2026), GAGgugaguu (SEQ ID NO: 2027), GAGgugauau (SEQ ID NO: 2028), GAGgugaucc (SEQ ID NO: 2029), GAGgugaucu (SEQ ID NO: 2030), GAGgugauga (SEQ ID NO: 2031), GAGgugaugg (SEQ ID NO: 2032), GAGgugaugu (SEQ ID NO: 2033), GAGgugauuc (SEQ ID NO: 2034), GAGgugcaca (SEQ ID NO: 2035), GAGgugcaga (SEQ ID NO: 2036), GAGgugcagc (SEQ ID NO: 2037), GAGgugcagg (SEQ ID NO: 2038), GAGgugccag (SEQ ID NO: 2039), GAGgugccca (SEQ ID NO: 2040), GAGgugccuu (SEQ ID NO: 2041), GAGgugcggg (SEQ ID NO: 2042), GAGgugcgug (SEQ ID NO: 2043), GAGgugcucc (SEQ ID NO: 2044), GAGgugcugg (SEQ ID NO: 2045), GAGgugcuua (SEQ ID NO: 2046), GAGgugcuug (SEQ ID NO: 2047), GAGguggaaa (SEQ ID NO: 2048), GAGguggaau (SEQ ID NO: 2049), GAGguggacc (SEQ ID NO: 2050), GAGguggacg (SEQ ID NO: 2051), GAGguggagg (SEQ ID NO: 2052), GAGguggcug (SEQ ID NO: 2053), GAGgugggaa (SEQ ID NO: 2054), GAGgugggag (SEQ ID NO: 2055), GAGgugggau (SEQ ID NO: 2056), GAGgugggca (SEQ ID NO: 2057), GAGgugggcg (SEQ ID NO: 2058), GAGgugggcu (SEQ ID NO: 2059), GAGgugggga (SEQ ID NO: 2060), GAGguggggc (SEQ ID NO: 2061), GAGguggggg (SEQ ID NO: 2062), GAGgugggua (SEQ ID NO: 2063), GAGguggguc (SEQ ID NO: 2064), GAGgugggug (SEQ ID NO: 2065), GAGguggguu (SEQ ID NO: 2066), GAGgugguau (SEQ ID NO: 2067), GAGgugguuc (SEQ ID NO: 2068), GAGgugucau (SEQ ID NO: 2069), GAGgugugag (SEQ ID NO: 2070), GAGgugugau (SEQ ID NO: 2071), GAGgugugca (SEQ ID NO: 2072), GAGgugugcu (SEQ ID NO: 2073), GAGgugugga (SEQ ID NO: 2074), GAGguguggg (SEQ ID NO: 2075), GAGguguggu (SEQ ID NO: 2076), GAGgugugua (SEQ ID NO: 2077), GAGgugugug (SEQ ID NO: 2078), GAGguuaaau (SEQ ID NO: 2079), GAGguuaaga (SEQ ID NO: 2080), GAGguuaaua (SEQ ID NO: 2081), GAGguuaccg (SEQ ID NO: 2082), GAGguuagaa (SEQ ID NO: 2083), GAGguuagac (SEQ ID NO: 2084), GAGguuagag (SEQ ID NO: 2085), GAGguuaggu (SEQ ID NO: 2086), GAGguuagua (SEQ ID NO: 2087), GAGguuaguc (SEQ ID NO: 2088), GAGguuagug (SEQ ID NO: 2089), GAGguuaguu (SEQ ID NO: 2090), GAGguuaugu (SEQ ID NO: 2091), GAGguuauuc (SEQ ID NO: 2092), GAGguucaaa (SEQ ID NO: 2093), GAGguucaua (SEQ ID NO: 2094), GAGguucuga (SEQ ID NO: 2095), GAGguugaag (SEQ ID NO: 2096), GAGguugcag (SEQ ID NO: 2097), GAGguugcug (SEQ ID NO: 2098), GAGguuggaa (SEQ ID NO: 2099), GAGguuggag (SEQ ID NO: 2100), GAGguuggau (SEQ ID NO: 2101), GAGguuggua (SEQ ID NO: 2102), GAGguugguc (SEQ ID NO: 2103), GAGguugguu (SEQ ID NO: 2104), GAGguuguag (SEQ ID NO: 2105), GAGguuucug (SEQ ID NO: 2106), GAGguuugag (SEQ ID NO: 2107), GAGguuugga (SEQ ID NO: 2108), GAGguuuggg (SEQ ID NO: 2109), GAGguuugua (SEQ ID NO: 2110), GAGguuuguu (SEQ ID NO: 2111), GAGguuuuca (SEQ ID NO: 2112), GAGguuuuga (SEQ ID NO: 2113), GAGguuuugg (SEQ ID NO: 2114), GAGguuuuua (SEQ ID NO: 2115), GAGguuuuuc (SEQ ID NO: 2116), GAUguaaaau (SEQ ID NO: 2117), GAUguaagca (SEQ ID NO: 2118), GAUguaagcc (SEQ ID NO: 2119), GAUguaaggu (SEQ ID NO: 2120), GAUguaagua (SEQ ID NO: 2121), GAUguaagug (SEQ ID NO: 2122), GAUguaaguu (SEQ ID NO: 2123), GAUguacauc (SEQ ID NO: 2124), GAUguaggua (SEQ ID NO: 2125), GAUguauggc (SEQ ID NO: 2126), GAUguaugua (SEQ ID NO: 2127), GAUguauguu (SEQ ID NO: 2128), GAUgucagug (SEQ ID NO: 2129), GAUgugagag (SEQ ID NO: 2130), GAUgugagcc (SEQ ID NO: 2131), GAUgugagcu (SEQ ID NO: 2132), GAUgugagga (SEQ ID NO: 2133), GAUgugaguc (SEQ ID NO: 2134), GAUgugagug (SEQ ID NO: 2135), GAUgugaguu (SEQ ID NO: 2136), GAUgugggua (SEQ ID NO: 2137), GAUgugggug (SEQ ID NO: 2138), GAUguguguu (SEQ ID NO: 2139), GAUguuagcu (SEQ ID NO: 2140), GAUguucagu (SEQ ID NO: 2141), GAUguucgug (SEQ ID NO: 2142), GAUguuuguu (SEQ ID NO: 2143), GCAguaaagg (SEQ ID NO: 2144), GCAguaagaa (SEQ ID NO: 2145), GCAguaagga (SEQ ID NO: 2146), GCAguaagua (SEQ ID NO: 2147), GCAguaaguc (SEQ ID NO: 2148), GCAguaaguu (SEQ ID NO: 2149), GCAguagaug (SEQ ID NO: 2150), GCAguaggua (SEQ ID NO: 2151), GCAguaugug (SEQ ID NO: 2152), GCAguauguu (SEQ ID NO: 2153), GCAgucagua (SEQ ID NO: 2154), GCAgucagug (SEQ ID NO: 2155), GCAguccggu (SEQ ID NO: 2156), GCAgugacuu (SEQ ID NO: 2157), GCAgugagcc (SEQ ID NO: 2158), GCAgugagcg (SEQ ID NO: 2159), GCAgugagcu (SEQ ID NO: 2160), GCAgugagua (SEQ ID NO: 2161), GCAgugagug (SEQ ID NO: 2162), GCAgugaguu (SEQ ID NO: 2163), GCAgugggua (SEQ ID NO: 2164), GCAguuaagu (SEQ ID NO: 2165), GCAguugagu (SEQ ID NO: 2166), GCCguaaguc (SEQ ID NO: 2167), GCCgugagua (SEQ ID NO: 2168), GCGguaaagc (SEQ ID NO: 2169), GCGguaaaua (SEQ ID NO: 2170), GCGguaagcu (SEQ ID NO: 2171), GCGguaaggg (SEQ ID NO: 2172), GCGguaagug (SEQ ID NO: 2173), GCGguaauca (SEQ ID NO: 2174), GCGguacgua (SEQ ID NO: 2175), GCGguacuug (SEQ ID NO: 2176), GCGguagggu (SEQ ID NO: 2177), GCGguagugu (SEQ ID NO: 2178), GCGgugagca (SEQ ID NO: 2179), GCGgugagcu (SEQ ID NO: 2180), GCGgugaguu (SEQ ID NO: 2181), GCGguggcuc (SEQ ID NO: 2182), GCGgugugca (SEQ ID NO: 2183), GCGguguguu (SEQ ID NO: 2184), GCGguuaagu (SEQ ID NO: 2185), GCGguuugca (SEQ ID NO: 2186), GCUgcuguaa (SEQ ID NO: 2187), GCUguaaaua (SEQ ID NO: 2188), GCUguaagac (SEQ ID NO: 2189), GCUguaagag (SEQ ID NO: 2190), GCUguaagca (SEQ ID NO: 2191), GCUguaagga (SEQ ID NO: 2192), GCUguaagua (SEQ ID NO: 2193), GCUguaaguc (SEQ ID NO: 2194), GCUguaagug (SEQ ID NO: 2195), GCUguaaguu (SEQ ID NO: 2196), GCUguaggug (SEQ ID NO: 2197), GCUguauggu (SEQ ID NO: 2198), GCUgucagug (SEQ ID NO: 2199), GCUguccuug (SEQ ID NO: 2200), GCUgugagaa (SEQ ID NO: 2201), GCUgugagcc (SEQ ID NO: 2202), GCUgugagga (SEQ ID NO: 2203), GCUgugagua (SEQ ID NO: 2204), GCUgugaguc (SEQ ID NO: 2205), GCUgugagug (SEQ ID NO: 2206), GCUgugaguu (SEQ ID NO: 2207), GCUguggguu (SEQ ID NO: 2208), GGAguaagag (SEQ ID NO: 2209), GGAguaagca (SEQ ID NO: 2210), GGAguaagcc (SEQ ID NO: 2211), GGAguaagcu (SEQ ID NO: 2212), GGAguaagga (SEQ ID NO: 2213), GGAguaagug (SEQ ID NO: 2214), GGAguaaguu (SEQ ID NO: 2215), GGAguaauuu (SEQ ID NO: 2216), GGAguacugu (SEQ ID NO: 2217), GGAguaggaa (SEQ ID NO: 2218), GGAguaggua (SEQ ID NO: 2219), GGAguagguu (SEQ ID NO: 2220), GGAguaguau (SEQ ID NO: 2221), GGAguaugac (SEQ ID NO: 2222), GGAguauggu (SEQ ID NO: 2223), GGAgucaagu (SEQ ID NO: 2224), GGAgugaggg (SEQ ID NO: 2225), GGAgugagua (SEQ ID NO: 2226), GGAgugaguc (SEQ ID NO: 2227), GGAgugagug (SEQ ID NO: 2228), GGAgugaguu (SEQ ID NO: 2229), GGAgugcuuu (SEQ ID NO: 2230), GGAgugggca (SEQ ID NO: 2231), GGAgugggug (SEQ ID NO: 2232), GGAguuaagg (SEQ ID NO: 2233), GGAguugaga (SEQ ID NO: 2234), GGCguaagcc (SEQ ID NO: 2235), GGCguaggua (SEQ ID NO: 2236), GGCguaggug (SEQ ID NO: 2237), GGCgugagcc (SEQ ID NO: 2238), GGCgugaguc (SEQ ID NO: 2239), GGGguaaaca (SEQ ID NO: 2240), GGGguaaacc (SEQ ID NO: 2241), GGGguaaacu (SEQ ID NO: 2242), GGGguaagaa (SEQ ID NO: 2243), GGGguaagag (SEQ ID NO: 2244), GGGguaagau (SEQ ID NO: 2245), GGGguaagca (SEQ ID NO: 2246), GGGguaagcc (SEQ ID NO: 2247), GGGguaagcu (SEQ ID NO: 2248), GGGguaagga (SEQ ID NO: 2249), GGGguaaggg (SEQ ID NO: 2250), GGGguaagua (SEQ ID NO: 2251), GGGguaagug (SEQ ID NO: 2252), GGGguaaguu (SEQ ID NO: 2253), GGGguagaca (SEQ ID NO: 2254), GGGguaggag (SEQ ID NO: 2255), GGGguaggcc (SEQ ID NO: 2256), GGGguaggga (SEQ ID NO: 2257), GGGguaggua (SEQ ID NO: 2258), GGGguaggug (SEQ ID NO: 2259), GGGguagguu (SEQ ID NO: 2260), GGGguagugc (SEQ ID NO: 2261), GGGguaucug (SEQ ID NO: 2262), GGGguaugac (SEQ ID NO: 2263), GGGguaugga (SEQ ID NO: 2264), GGGguaugua (SEQ ID NO: 2265), GGGguauguc (SEQ ID NO: 2266), GGGguaugug (SEQ ID NO: 2267), GGGguauguu (SEQ ID NO: 2268), GGGgucagua (SEQ ID NO: 2269), GGGguccgug (SEQ ID NO: 2270), GGGgucggag (SEQ ID NO: 2271), GGGgucugug (SEQ ID NO: 2272), GGGgugaaca (SEQ ID NO: 2273), GGGgugaaga (SEQ ID NO: 2274), GGGgugagaa (SEQ ID NO: 2275), GGGgugagau (SEQ ID NO: 2276), GGGgugagcc (SEQ ID NO: 2277), GGGgugagcg (SEQ ID NO: 2278), GGGgugagcu (SEQ ID NO: 2279), GGGgugagga (SEQ ID NO: 2280), GGGgugaggc (SEQ ID NO: 2281), GGGgugaggg (SEQ ID NO: 2282), GGGgugaguc (SEQ ID NO: 2283), GGGgugagug (SEQ ID NO: 2284), GGGgugaguu (SEQ ID NO: 2285), GGGgugcgua (SEQ ID NO: 2286), GGGguggggu (SEQ ID NO: 2287), GGGgugggua (SEQ ID NO: 2288), GGGgugggug (SEQ ID NO: 2289), GGGguggguu (SEQ ID NO: 2290), GGGgugugcg (SEQ ID NO: 2291), GGGgugugua (SEQ ID NO: 2292), GGGguguguc (SEQ ID NO: 2293), GGGgugugug (SEQ ID NO: 2294), GGGguuacag (SEQ ID NO: 2295), GGGguuggac (SEQ ID NO: 2296), GGGguuggga (SEQ ID NO: 2297), GGGguuugcc (SEQ ID NO: 2298), GGGguuugua (SEQ ID NO: 2299), GGUguaagaa (SEQ ID NO: 2300), GGUguaagau (SEQ ID NO: 2301), GGUguaagca (SEQ ID NO: 2302), GGUguaagcc (SEQ ID NO: 2303), GGUguaagcg (SEQ ID NO: 2304), GGUguaaguc (SEQ ID NO: 2305), GGUguaagug (SEQ ID NO: 2306), GGUguagguc (SEQ ID NO: 2307), GGUguaggug (SEQ ID NO: 2308), GGUguagguu (SEQ ID NO: 2309), GGUguccgua (SEQ ID NO: 2310), GGUgugagag (SEQ ID NO: 2311), GGUgugagcc (SEQ ID NO: 2312), GGUgugagcu (SEQ ID NO: 2313), GGUgugagua (SEQ ID NO: 2314), GGUgugaguc (SEQ ID NO: 2315), GGUgugcuuc (SEQ ID NO: 2316), GGUguggcug (SEQ ID NO: 2317), GGUgugguga (SEQ ID NO: 2318), GGUgugucug (SEQ ID NO: 2319), GGUguugaaa (SEQ ID NO: 2320), GGUguugcug (SEQ ID NO: 2321), GUAguaagau (SEQ ID NO: 2322), GUAguaagua (SEQ ID NO: 2323), GUAguaagug (SEQ ID NO: 2324), GUAguagcuu (SEQ ID NO: 2325), GUAguaggua (SEQ ID NO: 2326), GUAgucagua (SEQ ID NO: 2327), GUAgugagua (SEQ ID NO: 2328), GUAguggugg (SEQ ID NO: 2329), GUAguuaagu (SEQ ID NO: 2330), GUAguuucug (SEQ ID NO: 2331), GUCguaagug (SEQ ID NO: 2332), GUCgugagug (SEQ ID NO: 2333), GUCgugaguu (SEQ ID NO: 2334), GUGgcaagua (SEQ ID NO: 2335), GUGgcuugua (SEQ ID NO: 2336), GUGguaaaau (SEQ ID NO: 2337), GUGguaaaga (SEQ ID NO: 2338), GUGguaaauu (SEQ ID NO: 2339), GUGguaacau (SEQ ID NO: 2340), GUGguaacua (SEQ ID NO: 2341), GUGguaagaa (SEQ ID NO: 2342), GUGguaagac (SEQ ID NO: 2343), GUGguaagag (SEQ ID NO: 2344), GUGguaagau (SEQ ID NO: 2345), GUGguaagca (SEQ ID NO: 2346), GUGguaagcg (SEQ ID NO: 2347), GUGguaagcu (SEQ ID NO: 2348), GUGguaagga (SEQ ID NO: 2349), GUGguaaggc (SEQ ID NO: 2350), GUGguaagua (SEQ ID NO: 2351), GUGguaaguc (SEQ ID NO: 2352), GUGguaagug (SEQ ID NO: 2353), GUGguaaguu (SEQ ID NO: 2354), GUGguaauga (SEQ ID NO: 2355), GUGguaauuc (SEQ ID NO: 2356), GUGguaauuu (SEQ ID NO: 2357), GUGguacaug (SEQ ID NO: 2358), GUGguacgau (SEQ ID NO: 2359), GUGguacuau (SEQ ID NO: 2360), GUGguacuug (SEQ ID NO: 2361), GUGguagaua (SEQ ID NO: 2362), GUGguagege (SEQ ID NO: 2363), GUGguaggga (SEQ ID NO: 2364), GUGguagguc (SEQ ID NO: 2365), GUGguaggug (SEQ ID NO: 2366), GUGguagguu (SEQ ID NO: 2367), GUGguauaaa (SEQ ID NO: 2368), GUGguaucuc (SEQ ID NO: 2369), GUGguaugaa (SEQ ID NO: 2370), GUGguaugau (SEQ ID NO: 2371), GUGguaugca (SEQ ID NO: 2372), GUGguaugua (SEQ ID NO: 2373), GUGguauguu (SEQ ID NO: 2374), GUGguccgug (SEQ ID NO: 2375), GUGgucuggc (SEQ ID NO: 2376), GUGgugaaac (SEQ ID NO: 2377), GUGgugagaa (SEQ ID NO: 2378), GUGgugagau (SEQ ID NO: 2379), GUGgugagca (SEQ ID NO: 2380), GUGgugagcu (SEQ ID NO: 2381), GUGgugagga (SEQ ID NO: 2382), GUGgugaggc (SEQ ID NO: 2383), GUGgugagug (SEQ ID NO: 2384), GUGgugaguu (SEQ ID NO: 2385), GUGgugauua (SEQ ID NO: 2386), GUGgugauuc (SEQ ID NO: 2387), GUGgugcgau (SEQ ID NO: 2388), GUGgugcuua (SEQ ID NO: 2389), GUGgugggaa (SEQ ID NO: 2390), GUGgugggua (SEQ ID NO: 2391), GUGguggguc (SEQ ID NO: 2392), GUGguguccg (SEQ ID NO: 2393), GUGguuagca (SEQ ID NO: 2394), GUGguuaggu (SEQ ID NO: 2395), GUGguuagug (SEQ ID NO: 2396), GUGguuugca (SEQ ID NO: 2397), GUGguuugua (SEQ ID NO: 2398), GUUguaaggu (SEQ ID NO: 2399), GUUguaagua (SEQ ID NO: 2400), GUUguaaguc (SEQ ID NO: 2401), GUUguaaguu (SEQ ID NO: 2402), GUUguaccac (SEQ ID NO: 2403), GUUguagcgu (SEQ ID NO: 2404), GUUguaugug (SEQ ID NO: 2405), GUUguauguu (SEQ ID NO: 2406), GUUgucugug (SEQ ID NO: 2407), GUUgugagcu (SEQ ID NO: 2408), GUUgugagug (SEQ ID NO: 2409), GUUgugaguu (SEQ ID NO: 2410), GUUgugggua (SEQ ID NO: 2411), GUUguggguu (SEQ ID NO: 2412), UAAguaaaug (SEQ ID NO: 2413), UAAguaacua (SEQ ID NO: 2414), UAAguaagaa (SEQ ID NO: 2415), UAAguaagag (SEQ ID NO: 2416), UAAguaagau (SEQ ID NO: 2417), UAAguaagca (SEQ ID NO: 2418), UAAguaagcu (SEQ ID NO: 2419), UAAguaagga (SEQ ID NO: 2420), UAAguaaggu (SEQ ID NO: 2421), UAAguaagua (SEQ ID NO: 2422), UAAguaaguc (SEQ ID NO: 2423), UAAguaagug (SEQ ID NO: 2424), UAAguaaguu (SEQ ID NO: 2425), UAAguaauaa (SEQ ID NO: 2426), UAAguacuag (SEQ ID NO: 2427), UAAguaguuu (SEQ ID NO: 2428), UAAguauaaa (SEQ ID NO: 2429), UAAguauaca (SEQ ID NO: 2430), UAAguaugua (SEQ ID NO: 2431), UAAguauuau (SEQ ID NO: 2432), UAAguauuuu (SEQ ID NO: 2433), UAAgucuuuu (SEQ ID NO: 2434), UAAgugagac (SEQ ID NO: 2435), UAAgugagga (SEQ ID NO: 2436), UAAgugaggg (SEQ ID NO: 2437), UAAgugagua (SEQ ID NO: 2438), UAAgugaguc (SEQ ID NO: 2439), UAAgugagug (SEQ ID NO: 2440), UAAgugaguu (SEQ ID NO: 2441), UAAgugaucc (SEQ ID NO: 2442), UAAgugauuc (SEQ ID NO: 2443), UAAgugcgug (SEQ ID NO: 2444), UAAguuaagu (SEQ ID NO: 2445), UAAguuccag (SEQ ID NO: 2446), UAAguucuuu (SEQ ID NO: 2447), UAAguuguaa (SEQ ID NO: 2448), UAAguuguau (SEQ ID NO: 2449), UAAguuuguu (SEQ ID NO: 2450), UACguaacug (SEQ ID NO: 2451), UACguaagaa (SEQ ID NO: 2452), UACguaagau (SEQ ID NO: 2453), UACguaagua (SEQ ID NO: 2454), UACguaagug (SEQ ID NO: 2455), UACguauccu (SEQ ID NO: 2456), UACgucuggc (SEQ ID NO: 2457), UACgugacca (SEQ ID NO: 2458), UAGgcaagac (SEQ ID NO: 2459), UAGgcaaguc (SEQ ID NO: 2460), UAGgcagguc (SEQ ID NO: 2461), UAGgcgugug (SEQ ID NO: 2462), UAGguaaaaa (SEQ ID NO: 2463), UAGguaaaac (SEQ ID NO: 2464), UAGguaaaag (SEQ ID NO: 2465), UAGguaaaau (SEQ ID NO: 2466), UAGguaaaca (SEQ ID NO: 2467), UAGguaaaga (SEQ ID NO: 2468), UAGguaaaua (SEQ ID NO: 2469), UAGguaaauc (SEQ ID NO: 2470), UAGguaaaug (SEQ ID NO: 2471), UAGguaaauu (SEQ ID NO: 2472), UAGguaacac (SEQ ID NO: 2473), UAGguaacag (SEQ ID NO: 2474), UAGguaacau (SEQ ID NO: 2475), UAGguaacca (SEQ ID NO: 2476), UAGguaacgg (SEQ ID NO: 2477), UAGguaacua (SEQ ID NO: 2478), UAGguaacuc (SEQ ID NO: 2479), UAGguaacug (SEQ ID NO: 2480), UAGguaacuu (SEQ ID NO: 2481), UAGguaagac (SEQ ID NO: 2482), UAGguaagag (SEQ ID NO: 2483), UAGguaagau (SEQ ID NO: 2484), UAGguaagca (SEQ ID NO: 2485), UAGguaagcc (SEQ ID NO: 2486), UAGguaagcu (SEQ ID NO: 2487), UAGguaagga (SEQ ID NO: 2488), UAGguaaggc (SEQ ID NO: 2489), UAGguaaggg (SEQ ID NO: 2490), UAGguaagua (SEQ ID NO: 2491), UAGguaaguc (SEQ ID NO: 2492), UAGguaagug (SEQ ID NO: 2493), UAGguaaguu (SEQ ID NO: 2494), UAGguaauag (SEQ ID NO: 2495), UAGguaauau (SEQ ID NO: 2496), UAGguaaucu (SEQ ID NO: 2497), UAGguaauga (SEQ ID NO: 2498), UAGguaaugg (SEQ ID NO: 2499), UAGguaaugu (SEQ ID NO: 2500), UAGguaauua (SEQ ID NO: 2501), UAGguaauuc (SEQ ID NO: 2502), UAGguaauuu (SEQ ID NO: 2503), UAGguacagc (SEQ ID NO: 2504), UAGguacagu (SEQ ID NO: 2505), UAGguacauu (SEQ ID NO: 2506), UAGguaccag (SEQ ID NO: 2507), UAGguaccua (SEQ ID NO: 2508), UAGguaccuu (SEQ ID NO: 2509), UAGguacgag (SEQ ID NO: 2510), UAGguacgua (SEQ ID NO: 2511), UAGguacguu (SEQ ID NO: 2512), UAGguacuau (SEQ ID NO: 2513), UAGguacuga (SEQ ID NO: 2514), UAGguacugg (SEQ ID NO: 2515), UAGguacuuc (SEQ ID NO: 2516), UAGguacuuu (SEQ ID NO: 2517), UAGguagcgg (SEQ ID NO: 2518), UAGguaggaa (SEQ ID NO: 2519), UAGguaggac (SEQ ID NO: 2520), UAGguaggau (SEQ ID NO: 2521), UAGguaggga (SEQ ID NO: 2522), UAGguagggg (SEQ ID NO: 2523), UAGguaggua (SEQ ID NO: 2524), UAGguagguc (SEQ ID NO: 2525), UAGguaggug (SEQ ID NO: 2526), UAGguagguu (SEQ ID NO: 2527), UAGguaguaa (SEQ ID NO: 2528), UAGguagucu (SEQ ID NO: 2529), UAGguagugg (SEQ ID NO: 2530), UAGguagugu (SEQ ID NO: 2531), UAGguaguuu (SEQ ID NO: 2532), UAGguauaaa (SEQ ID NO: 2533), UAGguauaac (SEQ ID NO: 2534), UAGguauaag (SEQ ID NO: 2535), UAGguauaau (SEQ ID NO: 2536), UAGguauaca (SEQ ID NO: 2537), UAGguauacu (SEQ ID NO: 2538), UAGguauaua (SEQ ID NO: 2539), UAGguauauc (SEQ ID NO: 2540), UAGguauauu (SEQ ID NO: 2541), UAGguaucag (SEQ ID NO: 2542), UAGguaucua (SEQ ID NO: 2543), UAGguaucuc (SEQ ID NO: 2544), UAGguaugaa (SEQ ID NO: 2545), UAGguaugag (SEQ ID NO: 2546), UAGguaugca (SEQ ID NO: 2547), UAGguaugga (SEQ ID NO: 2548), UAGguauggc (SEQ ID NO: 2549), UAGguauggu (SEQ ID NO: 2550), UAGguaugua (SEQ ID NO: 2551), UAGguauguc (SEQ ID NO: 2552), UAGguaugug (SEQ ID NO: 2553), UAGguauguu (SEQ ID NO: 2554), UAGguauuaa (SEQ ID NO: 2555), UAGguauuac (SEQ ID NO: 2556), UAGguauuau (SEQ ID NO: 2557), UAGguauuca (SEQ ID NO: 2558), UAGguauucc (SEQ ID NO: 2559), UAGguauucu (SEQ ID NO: 2560), UAGguauuga (SEQ ID NO: 2561), UAGguauuua (SEQ ID NO: 2562), UAGguauuuc (SEQ ID NO: 2563), UAGguauuuu (SEQ ID NO: 2564), UAGgucacuc (SEQ ID NO: 2565), UAGgucagcu (SEQ ID NO: 2566), UAGgucaggu (SEQ ID NO: 2567), UAGgucagua (SEQ ID NO: 2568), UAGgucagug (SEQ ID NO: 2569), UAGgucaguu (SEQ ID NO: 2570), UAGgucaucu (SEQ ID NO: 2571), UAGgucauug (SEQ ID NO: 2572), UAGguccaau (SEQ ID NO: 2573), UAGguccugu (SEQ ID NO: 2574), UAGgucucaa (SEQ ID NO: 2575), UAGgucucgc (SEQ ID NO: 2576), UAGgucuggc (SEQ ID NO: 2577), UAGgucuguc (SEQ ID NO: 2578), UAGgucugug (SEQ ID NO: 2579), UAGgugaagu (SEQ ID NO: 2580), UAGgugaaua (SEQ ID NO: 2581), UAGgugaaug (SEQ ID NO: 2582), UAGgugaauu (SEQ ID NO: 2583), UAGgugacau (SEQ ID NO: 2584), UAGgugacca (SEQ ID NO: 2585), UAGgugacua (SEQ ID NO: 2586), UAGgugagaa (SEQ ID NO: 2587), UAGgugagac (SEQ ID NO: 2588), UAGgugagag (SEQ ID NO: 2589), UAGgugagau (SEQ ID NO: 2590), UAGgugagcc (SEQ ID NO: 2591), UAGgugagcu (SEQ ID NO: 2592), UAGgugagga (SEQ ID NO: 2593), UAGgugaggc (SEQ ID NO: 2594), UAGgugaggu (SEQ ID NO: 2595), UAGgugagua (SEQ ID NO: 2596), UAGgugaguc (SEQ ID NO: 2597), UAGgugagug (SEQ ID NO: 2598), UAGgugauca (SEQ ID NO: 2599), UAGgugauuc (SEQ ID NO: 2600), UAGgugauuu (SEQ ID NO: 2601), UAGgugcaua (SEQ ID NO: 2602), UAGgugcauc (SEQ ID NO: 2603), UAGgugccgu (SEQ ID NO: 2604), UAGgugccug (SEQ ID NO: 2605), UAGgugcgca (SEQ ID NO: 2606), UAGgugcgua (SEQ ID NO: 2607), UAGgugcgug (SEQ ID NO: 2608), UAGgugcuga (SEQ ID NO: 2609), UAGguggaua (SEQ ID NO: 2610), UAGgugggaa (SEQ ID NO: 2611), UAGgugggac (SEQ ID NO: 2612), UAGgugggag (SEQ ID NO: 2613), UAGgugggau (SEQ ID NO: 2614), UAGgugggcc (SEQ ID NO: 2615), UAGgugggcu (SEQ ID NO: 2616), UAGguggguu (SEQ ID NO: 2617), UAGguggugu (SEQ ID NO: 2618), UAGguguaaa (SEQ ID NO: 2619), UAGgugugaa (SEQ ID NO: 2620), UAGgugugag (SEQ ID NO: 2621), UAGgugugca (SEQ ID NO: 2622), UAGgugugcc (SEQ ID NO: 2623), UAGgugugcg (SEQ ID NO: 2624), UAGguguggu (SEQ ID NO: 2625), UAGgugugua (SEQ ID NO: 2626), UAGgugugug (SEQ ID NO: 2627), UAGguguugg (SEQ ID NO: 2628), UAGguuaagc (SEQ ID NO: 2629), UAGguuagac (SEQ ID NO: 2630), UAGguuagcc (SEQ ID NO: 2631), UAGguuaggc (SEQ ID NO: 2632), UAGguuagua (SEQ ID NO: 2633), UAGguuaguc (SEQ ID NO: 2634), UAGguuagug (SEQ ID NO: 2635), UAGguucccc (SEQ ID NO: 2636), UAGguucuac (SEQ ID NO: 2637), UAGguuggua (SEQ ID NO: 2638), UAGguugguu (SEQ ID NO: 2639), UAGguugucc (SEQ ID NO: 2640), UAGguuuauu (SEQ ID NO: 2641), UAGguuugcc (SEQ ID NO: 2642), UAGguuugua (SEQ ID NO: 2643), UAGguuuguc (SEQ ID NO: 2644), UAGguuugug (SEQ ID NO: 2645), UAGguuuguu (SEQ ID NO: 2646), UAGguuuuuc (SEQ ID NO: 2647), UAGguuuuug (SEQ ID NO: 2648), UAUguaagaa (SEQ ID NO: 2649), UAUguaagau (SEQ ID NO: 2650), UAUguaagca (SEQ ID NO: 2651), UAUguaagcc (SEQ ID NO: 2652), UAUguaagua (SEQ ID NO: 2653), UAUguaaguc (SEQ ID NO: 2654), UAUguaagug (SEQ ID NO: 2655), UAUguaaguu (SEQ ID NO: 2656), UAUguacgug (SEQ ID NO: 2657), UAUguacguu (SEQ ID NO: 2658), UAUguagguc (SEQ ID NO: 2659), UAUguagguu (SEQ ID NO: 2660), UAUguauccu (SEQ ID NO: 2661), UAUguaucuc (SEQ ID NO: 2662), UAUguaugua (SEQ ID NO: 2663), UAUguauguc (SEQ ID NO: 2664), UAUguaugug (SEQ ID NO: 2665), UAUguauuau (SEQ ID NO: 2666), UAUgucagaa (SEQ ID NO: 2667), UAUgucugua (SEQ ID NO: 2668), UAUgugaaua (SEQ ID NO: 2669), UAUgugacag (SEQ ID NO: 2670), UAUgugagua (SEQ ID NO: 2671), UAUgugagug (SEQ ID NO: 2672), UAUgugaguu (SEQ ID NO: 2673), UAUgugggca (SEQ ID NO: 2674), UAUgugugua (SEQ ID NO: 2675), UAUguguuua (SEQ ID NO: 2676), UAUguuuugu (SEQ ID NO: 2677), UCAgcgacau (SEQ ID NO: 2678), UCAguaaaau (SEQ ID NO: 2679), UCAguaaaua (SEQ ID NO: 2680), UCAguaacug (SEQ ID NO: 2681), UCAguaagaa (SEQ ID NO: 2682), UCAguaagag (SEQ ID NO: 2683), UCAguaagau (SEQ ID NO: 2684), UCAguaagca (SEQ ID NO: 2685), UCAguaagcc (SEQ ID NO: 2686), UCAguaagcu (SEQ ID NO: 2687), UCAguaaggg (SEQ ID NO: 2688), UCAguaagua (SEQ ID NO: 2689), UCAguaaguc (SEQ ID NO: 2690), UCAguaagug (SEQ ID NO: 2691), UCAguaaguu (SEQ ID NO: 2692), UCAguaucuu (SEQ ID NO: 2693), UCAguaugga (SEQ ID NO: 2694), UCAguauggu (SEQ ID NO: 2695), UCAgucccca (SEQ ID NO: 2696), UCAgugagca (SEQ ID NO: 2697), UCAgugagcu (SEQ ID NO: 2698), UCAgugagua (SEQ ID NO: 2699), UCAgugagug (SEQ ID NO: 2700), UCAgugaguu (SEQ ID NO: 2701), UCAgugauug (SEQ ID NO: 2702), UCAgugggug (SEQ ID NO: 2703), UCAguugagc (SEQ ID NO: 2704), UCAguugauu (SEQ ID NO: 2705), UCAguuuagu (SEQ ID NO: 2706), UCCguaagca (SEQ ID NO: 2707), UCCguaagcu (SEQ ID NO: 2708), UCCguaaguc (SEQ ID NO: 2709), UCCguaagug (SEQ ID NO: 2710), UCCguaauag (SEQ ID NO: 2711), UCCguacuua (SEQ ID NO: 2712), UCCguaugua (SEQ ID NO: 2713), UCCguauguu (SEQ ID NO: 2714), UCCgugagau (SEQ ID NO: 2715), UCCgugaguc (SEQ ID NO: 2716), UCGguaaauu (SEQ ID NO: 2717), UCGguaagag (SEQ ID NO: 2718), UCGguaagcu (SEQ ID NO: 2719), UCGguacauc (SEQ ID NO: 2720), UCGguacucc (SEQ ID NO: 2721), UCGguagacc (SEQ ID NO: 2722), UCGguagguu (SEQ ID NO: 2723), UCGguaguaa (SEQ ID NO: 2724), UCGguaugug (SEQ ID NO: 2725), UCGguauguu (SEQ ID NO: 2726), UCGguauuga (SEQ ID NO: 2727), UCGgucagua (SEQ ID NO: 2728), UCGgucuuag (SEQ ID NO: 2729), UCGgugaagu (SEQ ID NO: 2730), UCGgugagaa (SEQ ID NO: 2731), UCGgugagca (SEQ ID NO: 2732), UCGgugaggc (SEQ ID NO: 2733), UCGgugagua (SEQ ID NO: 2734), UCGgugcgcu (SEQ ID NO: 2735), UCGgugcuuu (SEQ ID NO: 2736), UCGgugguuu (SEQ ID NO: 2737), UCGguuagcu (SEQ ID NO: 2738), UCUguaaaag (SEQ ID NO: 2739), UCUguaagaa (SEQ ID NO: 2740), UCUguaagau (SEQ ID NO: 2741), UCUguaagca (SEQ ID NO: 2742), UCUguaagcu (SEQ ID NO: 2743), UCUguaagua (SEQ ID NO: 2744), UCUguaaguc (SEQ ID NO: 2745), UCUguaagug (SEQ ID NO: 2746), UCUguaaguu (SEQ ID NO: 2747), UCUguaauaa (SEQ ID NO: 2748), UCUguaauga (SEQ ID NO: 2749), UCUguaaugu (SEQ ID NO: 2750), UCUguaggua (SEQ ID NO: 2751), UCUguagguu (SEQ ID NO: 2752), UCUguauaua (SEQ ID NO: 2753), UCUguaugac (SEQ ID NO: 2754), UCUguaugua (SEQ ID NO: 2755), UCUguccucg (SEQ ID NO: 2756), UCUgugagag (SEQ ID NO: 2757), UCUgugagcu (SEQ ID NO: 2758), UCUgugagga (SEQ ID NO: 2759), UCUgugagua (SEQ ID NO: 2760), UCUgugaguc (SEQ ID NO: 2761), UCUgugagug (SEQ ID NO: 2762), UCUgugaguu (SEQ ID NO: 2763), UCUgugcgua (SEQ ID NO: 2764), UCUgugugag (SEQ ID NO: 2765), UGAguaacuu (SEQ ID NO: 2766), UGAguaagau (SEQ ID NO: 2767), UGAguaagca (SEQ ID NO: 2768), UGAguaagcu (SEQ ID NO: 2769), UGAguaaggc (SEQ ID NO: 2770), UGAguaaggu (SEQ ID NO: 2771), UGAguaagua (SEQ ID NO: 2772), UGAguaaguc (SEQ ID NO: 2773), UGAguaagug (SEQ ID NO: 2774), UGAguaaguu (SEQ ID NO: 2775), UGAguaaucc (SEQ ID NO: 2776), UGAguaauua (SEQ ID NO: 2777), UGAguacagu (SEQ ID NO: 2778), UGAguacgua (SEQ ID NO: 2779), UGAguacguu (SEQ ID NO: 2780), UGAguacugu (SEQ ID NO: 2781), UGAguagcug (SEQ ID NO: 2782), UGAguaggua (SEQ ID NO: 2783), UGAguauaaa (SEQ ID NO: 2784), UGAguaugcu (SEQ ID NO: 2785), UGAguaugga (SEQ ID NO: 2786), UGAguaugua (SEQ ID NO: 2787), UGAguauguc (SEQ ID NO: 2788), UGAguauguu (SEQ ID NO: 2789), UGAgucagag (SEQ ID NO: 2790), UGAgucuacg (SEQ ID NO: 2791), UGAgugaaua (SEQ ID NO: 2792), UGAgugaauu (SEQ ID NO: 2793), UGAgugagaa (SEQ ID NO: 2794), UGAgugagau (SEQ ID NO: 2795), UGAgugagca (SEQ ID NO: 2796), UGAgugagcc (SEQ ID NO: 2797), UGAgugagga (SEQ ID NO: 2798), UGAgugagua (SEQ ID NO: 2799), UGAgugagug (SEQ ID NO: 2800), UGAgugaguu (SEQ ID NO: 2801), UGAgugggaa (SEQ ID NO: 2802), UGAguuaaga (SEQ ID NO: 2803), UGAguuaaug (SEQ ID NO: 2804), UGAguuacgg (SEQ ID NO: 2805), UGAguuaggu (SEQ ID NO: 2806), UGAguucuau (SEQ ID NO: 2807), UGAguugguu (SEQ ID NO: 2808), UGAguuguag (SEQ ID NO: 2809), UGAguuuauc (SEQ ID NO: 2810), UGCguaaguc (SEQ ID NO: 2811), UGCguaagug (SEQ ID NO: 2812), UGCguacggc (SEQ ID NO: 2813), UGCguacggg (SEQ ID NO: 2814), UGCguaugua (SEQ ID NO: 2815), UGGgcaaguc (SEQ ID NO: 2816), UGGgcaagug (SEQ ID NO: 2817), UGGgcacauc (SEQ ID NO: 2818), UGGgccacgu (SEQ ID NO: 2819), UGGgccccgg (SEQ ID NO: 2820), UGGguaaaau (SEQ ID NO: 2821), UGGguaaagc (SEQ ID NO: 2822), UGGguaaagg (SEQ ID NO: 2823), UGGguaaagu (SEQ ID NO: 2824), UGGguaaaua (SEQ ID NO: 2825), UGGguaaaug (SEQ ID NO: 2826), UGGguaaauu (SEQ ID NO: 2827), UGGguaacag (SEQ ID NO: 2828), UGGguaacau (SEQ ID NO: 2829), UGGguaacua (SEQ ID NO: 2830), UGGguaacuu (SEQ ID NO: 2831), UGGguaagaa (SEQ ID NO: 2832), UGGguaagac (SEQ ID NO: 2833), UGGguaagag (SEQ ID NO: 2834), UGGguaagau (SEQ ID NO: 2835), UGGguaagca (SEQ ID NO: 2836), UGGguaagcc (SEQ ID NO: 2837), UGGguaagcu (SEQ ID NO: 2838), UGGguaaggg (SEQ ID NO: 2839), UGGguaaggu (SEQ ID NO: 2840), UGGguaagua (SEQ ID NO: 2841), UGGguaaguc (SEQ ID NO: 2842), UGGguaagug (SEQ ID NO: 2843), UGGguaaguu (SEQ ID NO: 2844), UGGguaaugu (SEQ ID NO: 2845), UGGguaauua (SEQ ID NO: 2846), UGGguaauuu (SEQ ID NO: 2847), UGGguacaaa (SEQ ID NO: 2848), UGGguacagu (SEQ ID NO: 2849), UGGguacuac (SEQ ID NO: 2850), UGGguaggga (SEQ ID NO: 2851), UGGguagguc (SEQ ID NO: 2852), UGGguaggug (SEQ ID NO: 2853), UGGguagguu (SEQ ID NO: 2854), UGGguaguua (SEQ ID NO: 2855), UGGguauagu (SEQ ID NO: 2856), UGGguaugaa (SEQ ID NO: 2857), UGGguaugac (SEQ ID NO: 2858), UGGguaugag (SEQ ID NO: 2859), UGGguaugua (SEQ ID NO: 2860), UGGguauguc (SEQ ID NO: 2861), UGGguaugug (SEQ ID NO: 2862), UGGguauguu (SEQ ID NO: 2863), UGGguauuug (SEQ ID NO: 2864), UGGgucuuug (SEQ ID NO: 2865), UGGgugaccu (SEQ ID NO: 2866), UGGgugacua (SEQ ID NO: 2867), UGGgugagac (SEQ ID NO: 2868), UGGgugagag (SEQ ID NO: 2869), UGGgugagca (SEQ ID NO: 2870), UGGgugagcc (SEQ ID NO: 2871), UGGgugagga (SEQ ID NO: 2872), UGGgugaggc (SEQ ID NO: 2873), UGGgugaggg (SEQ ID NO: 2874), UGGgugagua (SEQ ID NO: 2875), UGGgugaguc (SEQ ID NO: 2876), UGGgugagug (SEQ ID NO: 2877), UGGgugaguu (SEQ ID NO: 2878), UGGgugcgug (SEQ ID NO: 2879), UGGguggagg (SEQ ID NO: 2880), UGGguggcuu (SEQ ID NO: 2881), UGGguggggg (SEQ ID NO: 2882), UGGgugggua (SEQ ID NO: 2883), UGGguggguc (SEQ ID NO: 2884), UGGgugggug (SEQ ID NO: 2885), UGGguggguu (SEQ ID NO: 2886), UGGgugugga (SEQ ID NO: 2887), UGGguguguc (SEQ ID NO: 2888), UGGgugugug (SEQ ID NO: 2889), UGGguguguu (SEQ ID NO: 2890), UGGguguuua (SEQ ID NO: 2891), UGGguuaaug (SEQ ID NO: 2892), UGGguuaguc (SEQ ID NO: 2893), UGGguuagug (SEQ ID NO: 2894), UGGguuaguu (SEQ ID NO: 2895), UGGguucaag (SEQ ID NO: 2896), UGGguucgua (SEQ ID NO: 2897), UGGguuggug (SEQ ID NO: 2898), UGGguuuaag (SEQ ID NO: 2899), UGGguuugua (SEQ ID NO: 2900), UGUgcaagua (SEQ ID NO: 2901), UGUguaaaua (SEQ ID NO: 2902), UGUguaagaa (SEQ ID NO: 2903), UGUguaagac (SEQ ID NO: 2904), UGUguaagag (SEQ ID NO: 2905), UGUguaaggu (SEQ ID NO: 2906), UGUguaagua (SEQ ID NO: 2907), UGUguaaguc (SEQ ID NO: 2908), UGUguaaguu (SEQ ID NO: 2909), UGUguacuuc (SEQ ID NO: 2910), UGUguaggeg (SEQ ID NO: 2911), UGUguaggua (SEQ ID NO: 2912), UGUguaguua (SEQ ID NO: 2913), UGUguaugug (SEQ ID NO: 2914), UGUgucagua (SEQ ID NO: 2915), UGUgucugua (SEQ ID NO: 2916), UGUgucuguc (SEQ ID NO: 2917), UGUgugaccc (SEQ ID NO: 2918), UGUgugagau (SEQ ID NO: 2919), UGUgugagca (SEQ ID NO: 2920), UGUgugagcc (SEQ ID NO: 2921), UGUgugagua (SEQ ID NO: 2922), UGUgugaguc (SEQ ID NO: 2923), UGUgugagug (SEQ ID NO: 2924), UGUgugcgug (SEQ ID NO: 2925), UGUgugggug (SEQ ID NO: 2926), UGUguggguu (SEQ ID NO: 2927), UGUgugugag (SEQ ID NO: 2928), UGUguguucu (SEQ ID NO: 2929), UGUguuuaga (SEQ ID NO: 2930), UUAguaaaua (SEQ ID NO: 2931), UUAguaagaa (SEQ ID NO: 2932), UUAguaagua (SEQ ID NO: 2933), UUAguaagug (SEQ ID NO: 2934), UUAguaaguu (SEQ ID NO: 2935), UUAguaggug (SEQ ID NO: 2936), UUAgugagca (SEQ ID NO: 2937), UUAgugaguu (SEQ ID NO: 2938), UUAguuaagu (SEQ ID NO: 2939), UUCguaaguc (SEQ ID NO: 2940), UUCguaaguu (SEQ ID NO: 2941), UUCguaauua (SEQ ID NO: 2942), UUCgugagua (SEQ ID NO: 2943), UUCgugaguu (SEQ ID NO: 2944), UUGgcaagug (SEQ ID NO: 2945), UUGgccgagu (SEQ ID NO: 2946), UUGguaaaaa (SEQ ID NO: 2947), UUGguaaaau (SEQ ID NO: 2948), UUGguaaaga (SEQ ID NO: 2949), UUGguaaagg (SEQ ID NO: 2950), UUGguaaagu (SEQ ID NO: 2951), UUGguaaauc (SEQ ID NO: 2952), UUGguaaaug (SEQ ID NO: 2953), UUGguaaauu (SEQ ID NO: 2954), UUGguaacug (SEQ ID NO: 2955), UUGguaacuu (SEQ ID NO: 2956), UUGguaagaa (SEQ ID NO: 2957), UUGguaagag (SEQ ID NO: 2958), UUGguaagcu (SEQ ID NO: 2959), UUGguaagga (SEQ ID NO: 2960), UUGguaaggg (SEQ ID NO: 2961), UUGguaagua (SEQ ID NO: 2962), UUGguaagug (SEQ ID NO: 2963), UUGguaaguu (SEQ ID NO: 2964), UUGguaauac (SEQ ID NO: 2965), UUGguaauca (SEQ ID NO: 2966), UUGguaaugc (SEQ ID NO: 2967), UUGguaaugu (SEQ ID NO: 2968), UUGguaauug (SEQ ID NO: 2969), UUGguaauuu (SEQ ID NO: 2970), UUGguacaua (SEQ ID NO: 2971), UUGguacgug (SEQ ID NO: 2972), UUGguagagg (SEQ ID NO: 2973), UUGguaggac (SEQ ID NO: 2974), UUGguaggcg (SEQ ID NO: 2975), UUGguaggcu (SEQ ID NO: 2976), UUGguaggga (SEQ ID NO: 2977), UUGguaggua (SEQ ID NO: 2978), UUGguagguc (SEQ ID NO: 2979), UUGguaggug (SEQ ID NO: 2980), UUGguauaaa (SEQ ID NO: 2981), UUGguauaca (SEQ ID NO: 2982), UUGguauauu (SEQ ID NO: 2983), UUGguaucua (SEQ ID NO: 2984), UUGguaucuc (SEQ ID NO: 2985), UUGguaugca (SEQ ID NO: 2986), UUGguaugua (SEQ ID NO: 2987), UUGguaugug (SEQ ID NO: 2988), UUGguauguu (SEQ ID NO: 2989), UUGguauugu (SEQ ID NO: 2990), UUGguauuua (SEQ ID NO: 2991), UUGguauuuu (SEQ ID NO: 2992), UUGgucagaa (SEQ ID NO: 2993), UUGgucagua (SEQ ID NO: 2994), UUGgucucug (SEQ ID NO: 2995), UUGgucugca (SEQ ID NO: 2996), UUGgugaaaa (SEQ ID NO: 2997), UUGgugacug (SEQ ID NO: 2998), UUGgugagac (SEQ ID NO: 2999), UUGgugagau (SEQ ID NO: 3000), UUGgugagca (SEQ ID NO: 3001), UUGgugagga (SEQ ID NO: 3002), UUGgugaggg (SEQ ID NO: 3003), UUGgugagua (SEQ ID NO: 3004), UUGgugaguc (SEQ ID NO: 3005), UUGgugagug (SEQ ID NO: 3006), UUGgugaguu (SEQ ID NO: 3007), UUGgugaugg (SEQ ID NO: 3008), UUGgugauua (SEQ ID NO: 3009), UUGgugauug (SEQ ID NO: 3010), UUGgugcaca (SEQ ID NO: 3011), UUGgugggaa (SEQ ID NO: 3012), UUGguggggc (SEQ ID NO: 3013), UUGgugggua (SEQ ID NO: 3014), UUGguggguc (SEQ ID NO: 3015), UUGgugggug (SEQ ID NO: 3016), UUGguggguu (SEQ ID NO: 3017), UUGguguggu (SEQ ID NO: 3018), UUGguguguc (SEQ ID NO: 3019), UUGgugugug (SEQ ID NO: 3020), UUGguguguu (SEQ ID NO: 3021), UUGguuaagu (SEQ ID NO: 3022), UUGguuagca (SEQ ID NO: 3023), UUGguuagug (SEQ ID NO: 3024), UUGguuaguu (SEQ ID NO: 3025), UUGguuggga (SEQ ID NO: 3026), UUGguugguu (SEQ ID NO: 3027), UUGguuugua (SEQ ID NO: 3028), UUGguuuguc (SEQ ID NO: 3029), UUUgcaagug (SEQ ID NO: 3030), UUUguaaaua (SEQ ID NO: 3031), UUUguaaaug (SEQ ID NO: 3032), UUUguaagaa (SEQ ID NO: 3033), UUUguaagac (SEQ ID NO: 3034), UUUguaagag (SEQ ID NO: 3035), UUUguaagca (SEQ ID NO: 3036), UUUguaaggu (SEQ ID NO: 3037), UUUguaagua (SEQ ID NO: 3038), UUUguaaguc (SEQ ID NO: 3039), UUUguaagug (SEQ ID NO: 3040), UUUguaaguu (SEQ ID NO: 3041), UUUguaauuu (SEQ ID NO: 3042), UUUguacagg (SEQ ID NO: 3043), UUUguacgug (SEQ ID NO: 3044), UUUguacuag (SEQ ID NO: 3045), UUUguacugu (SEQ ID NO: 3046), UUUguagguu (SEQ ID NO: 3047), UUUguauccu (SEQ ID NO: 3048), UUUguauguu (SEQ ID NO: 3049), UUUgugagca (SEQ ID NO: 3050), UUUgugagug (SEQ ID NO: 3051), UUUgugcguc (SEQ ID NO: 3052), UUUguguguc (SEQ ID NO: 3053), and uGGguaccug (SEQ ID NO: 3054).

Additional exemplary gene sequences and splice site sequences (e.g., 5′ splice site sequences) include AAGgcaagau (SEQ ID NO: 96), AUGguaugug (SEQ ID NO: 937), GGGgugaggc (SEQ ID NO: 2281), CAGguaggug (SEQ ID NO: 1222), AAGgucagua (SEQ ID NO: 293), AAGguuagag (SEQ ID NO: 3055), AUGgcacuua (SEQ ID NO: 3056), UAAguaaguc (SEQ ID NO: 2423), UGGgugagcu (SEQ ID NO: 3057), CGAgcugggc (SEQ ID NO: 3058), AAAgcacccc (SEQ ID NO: 3059), UAGguggggg (SEQ ID NO: 3060), AGAguaacgu (SEQ ID NO: 3061), UCGgugaugu (SEQ ID NO: 3062), AAUgucaguu (SEQ ID NO: 516), AGGgucugag (SEQ ID NO: 3063), GAGgugacug (SEQ ID NO: 3064), AUGguagguu (SEQ ID NO: 3065), GAGgucuguc (SEQ ID NO: 2000), CAGguaugug (SEQ ID NO: 1260), CAAguacugc (SEQ ID NO: 3066), CACgugcgua (SEQ ID NO: 3067), CCGgugagcu (SEQ ID NO: 3068), CAGguacuuc (SEQ ID NO: 3069), CAGgcgagag (SEQ ID NO: 1115), GAAgcaagua (SEQ ID NO: 3070), AGGgugagca (SEQ ID NO: 789), CAGgcaaguc (SEQ ID NO: 3071), AAGgugaggc (SEQ ID NO: 344), CAGguaagua (SEQ ID NO: 1147), CCAguugggu (SEQ ID NO: 3072), AAGguguggg (SEQ ID NO: 3073), CAGguuggag (SEQ ID NO: 1484), CCGguaugaa (SEQ ID NO: 3074), UGGguaaugu (SEQ ID NO: 2845), CAGgugaggu (SEQ ID NO: 1344), AGAguaauag (SEQ ID NO: 3075), CAGguaugag (SEQ ID NO: 1249), AUGguaaguu (SEQ ID NO: 901), UUGguggguc (SEQ ID NO: 3015), UUUguaagca (SEQ ID NO: 3036), CUCguaugcc (SEQ ID NO: 3076), UAGguaagag (SEQ ID NO: 2483), UAGgcaaguu (SEQ ID NO: 3077), GGAguuaagu (SEQ ID NO: 3078), GAGguaugcc (SEQ ID NO: 1959), AAGguguggu (SEQ ID NO: 402), CAGgugggug (SEQ ID NO: 1415), UUAguaagua (SEQ ID NO: 2933), AAGguuggcu (SEQ ID NO: 3079), UGAguaugug (SEQ ID NO: 3080), CCAgccuucc (SEQ ID NO: 3081), CCUguacgug (SEQ ID NO: 3082), CCUguaggua (SEQ ID NO: 1601), CAGguacgcu (SEQ ID NO: 3083), GAGguucuuc (SEQ ID NO: 3084), AAGguugccu (SEQ ID NO: 3085), CGUguucacu (SEQ ID NO: 3086), CGGgugggga (SEQ ID NO: 3087), UAGgugggau (SEQ ID NO: 2614), CGGguaagga (SEQ ID NO: 3088), AAGguacuau (SEQ ID NO: 195), GGGguaagcu (SEQ ID NO: 2248), ACGguagagc (SEQ ID NO: 3089), CAGgugaaga (SEQ ID NO: 1318), GCGguaagag (SEQ ID NO: 3090), CAGguguugu (SEQ ID NO: 3091), GAAguuugug (SEQ ID NO: 3092), AUGgugagca (SEQ ID NO: 955), CGGguucgug (SEQ ID NO: 3093), AUUguccggc (SEQ ID NO: 3094), GAUgugugug (SEQ ID NO: 3095), AUGgucuguu (SEQ ID NO: 3096), AAGguaggau (SEQ ID NO: 219), CCGguaagau (SEQ ID NO: 1575), AAGguaaaga (SEQ ID NO: 126), GGGgugaguu (SEQ ID NO: 2285), AGGguuggug (SEQ ID NO: 808), GGAgugagug (SEQ ID NO: 2228), AGUguaagga (SEQ ID NO: 3097), UAGguaacug (SEQ ID NO: 2480), AAGgugaaga (SEQ ID NO: 3098), UGGguaagug (SEQ ID NO: 2843), CAGguaagag (SEQ ID NO: 1140), UAGgugagcg (SEQ ID NO: 3099), GAGguaaaaa (SEQ ID NO: 1865), GCCguaaguu (SEQ ID NO: 3100), AAGguuuugu (SEQ ID NO: 473), CAGgugagga (SEQ ID NO: 1341), ACAgcccaug (SEQ ID NO: 3101), GCGgugagcc (SEQ ID NO: 3102), CAGguaugca (SEQ ID NO: 1251), AUGguaccua (SEQ ID NO: 3103), CAAguaugua (SEQ ID NO: 1050), AUGgugguge (SEQ ID NO: 3104), UAAguggcag (SEQ ID NO: 3105), UAGguauagu (SEQ ID NO: 3106), CUGguauuua (SEQ ID NO: 3107), AGGguaaacg (SEQ ID NO: 3108), AUAguaagug (SEQ ID NO: 850), UUGguacuga (SEQ ID NO: 3109), GGUguaagcc (SEQ ID NO: 2303), GAGguggaua (SEQ ID NO: 3110), GAUguaagaa (SEQ ID NO: 3111), ACGgucaguu (SEQ ID NO: 3112), UAAguaaaca (SEQ ID NO: 3113), AAGguaucug (SEQ ID NO: 251), AGGguauuug (SEQ ID NO: 3114), AAGgugaaug (SEQ ID NO: 328), CUGgugaauu (SEQ ID NO: 1749), CAGguuuuuu (SEQ ID NO: 1514), CAUguaugug (SEQ ID NO: 1534), UUGguagagg (SEQ ID NO: 2973), AAGguaugcc (SEQ ID NO: 258), CAGgugccac (SEQ ID NO: 3115), UCGguauuga (SEQ ID NO: 2727), AAGguuugug (SEQ ID NO: 468), AAUguacagg (SEQ ID NO: 3116), CAUguggguu (SEQ ID NO: 1545), CAUgugaguu (SEQ ID NO: 1542), UUGguaaugu (SEQ ID NO: 2968), AGUguaggug (SEQ ID NO: 3117), GAGguaacuc (SEQ ID NO: 3118), GAGguggcgc (SEQ ID NO: 3119), CUGguaauug (SEQ ID NO: 3120), GAGguuugcu (SEQ ID NO: 3121), UGUguacgug (SEQ ID NO: 3122), UAGguaaaga (SEQ ID NO: 2468), CUAguaggca (SEQ ID NO: 3123), UCUgugaguc (SEQ ID NO: 2761), UCUguaaggc (SEQ ID NO: 3124), CAGguuugug (SEQ ID NO: 1509), GAGguagggc (SEQ ID NO: 1935), AAGguaacca (SEQ ID NO: 3125), ACUgugaguu (SEQ ID NO: 646), UAGguaauag (SEQ ID NO: 2495), AAAguaagcu (SEQ ID NO: 17), AUGgugagug (SEQ ID NO: 963), UAGguuugug (SEQ ID NO: 2645), AACguaggac (SEQ ID NO: 3126), GUAgcaggua (SEQ ID NO: 3127), GAGgucagac (SEQ ID NO: 3128), AGGguaugaa (SEQ ID NO: 3129), GAGguuagug (SEQ ID NO: 2089), CAGgcacgug (SEQ ID NO: 3130), GGGgcaagac (SEQ ID NO: 3131), CAGguguguc (SEQ ID NO: 1441), CAGguauuga (SEQ ID NO: 1265), CAGguauguc (SEQ ID NO: 1259), AAGgcaaggu (SEQ ID NO: 3132), UUGgugagaa (SEQ ID NO: 3133), AAGguaaaau (SEQ ID NO: 122), GGGguaagua (SEQ ID NO: 2251), AAGguaucuu (SEQ ID NO: 252), GACgugaguc (SEQ ID NO: 3134), UAUguaugcu (SEQ ID NO: 3135), AAGguacugu (SEQ ID NO: 199), CAGgugaacu (SEQ ID NO: 3136), CACguaaaug (SEQ ID NO: 3137), AAGgugugau (SEQ ID NO: 3138), GAAguauuug (SEQ ID NO: 3139), AAGgucugug (SEQ ID NO: 3140), AAGguggagg (SEQ ID NO: 3141), AAGguauaug (SEQ ID NO: 244), CAGguucuua (SEQ ID NO: 1477), AGGguaacca (SEQ ID NO: 730), CAGgugucac (SEQ ID NO: 1423), AAAguucugu (SEQ ID NO: 3142), UUGgugaguu (SEQ ID NO: 3007), CAAgugaguc (SEQ ID NO: 1067), UAGguagguc (SEQ ID NO: 2525), GCGgugagcu (SEQ ID NO: 2180), AUUgugagga (SEQ ID NO: 3143), CAGgugcaca (SEQ ID NO: 1361), CAGguuggaa (SEQ ID NO: 3144), CUGgucacuu (SEQ ID NO: 3145), GGAguaagug (SEQ ID NO: 2214), GAGgugggcu (SEQ ID NO: 2059), AAGguacuug (SEQ ID NO: 201), AGGguaggau (SEQ ID NO: 3146), AAUguguguu (SEQ ID NO: 3147), ACAguuaagu (SEQ ID NO: 568), GAGgugugug (SEQ ID NO: 2078), AAGgcgggcu (SEQ ID NO: 3148), AUAgcaagua (SEQ ID NO: 3149), AAGguuguua (SEQ ID NO: 454), CAAgcaaggc (SEQ ID NO: 3150), GUGguaauua (SEQ ID NO: 3151), UCUguucagu (SEQ ID NO: 3152), AGGguaggcc (SEQ ID NO: 754), AAGguaucau (SEQ ID NO: 3153), UAGguaccuu (SEQ ID NO: 2509), AAGguaugac (SEQ ID NO: 254), GGAguaggua (SEQ ID NO: 2219), UAAguuggca (SEQ ID NO: 3154), AGUgugagge (SEQ ID NO: 3155), GAGguuugug (SEQ ID NO: 3156), UGGgucugcu (SEQ ID NO: 3157), CAGgugaucc (SEQ ID NO: 1350), CAGgucagug (SEQ ID NO: 1283), AAGguaaggg (SEQ ID NO: 151), CAGgugcagu (SEQ ID NO: 3158), GAGguggguc (SEQ ID NO: 2064), GCUgugagug (SEQ ID NO: 2206), AAGguggagu (SEQ ID NO: 3159), GGGgucaguu (SEQ ID NO: 3160), AGCguaagug (SEQ ID NO: 719), AGAguaugaa (SEQ ID NO: 691), GGGguagggu (SEQ ID NO: 3161), AAGgccagca (SEQ ID NO: 3162), CGAguaugcc (SEQ ID NO: 3163), GUGgugageg (SEQ ID NO: 3164), AAUguaaauu (SEQ ID NO: 481), CAGgugcgca (SEQ ID NO: 1375), GGUguaugaa (SEQ ID NO: 3165), CUUgugaguu (SEQ ID NO: 1804), AAGguaucuc (SEQ ID NO: 250), AGAguaagga (SEQ ID NO: 665), UAGguaagac (SEQ ID NO: 2482), GAGgugagug (SEQ ID NO: 2026), CAGguguguu (SEQ ID NO: 1443), UUGgugagua (SEQ ID NO: 3004), AGGgcgaguu (SEQ ID NO: 3166), CAGguuuugc (SEQ ID NO: 3167), UUUgugaguu (SEQ ID NO: 3168), AGGguaagca (SEQ ID NO: 736), GAGguccucu (SEQ ID NO: 3169), CCAgcaggua (SEQ ID NO: 3170), GAGguucgcg (SEQ ID NO: 3171), CAGgugaucu (SEQ ID NO: 1351), ACUguaagua (SEQ ID NO: 625), AAGguaaauc (SEQ ID NO: 131), CAGgcaaaua (SEQ ID NO: 3172), GUGguaagca (SEQ ID NO: 2346), CAGguuaaau (SEQ ID NO: 3173), UUGguaauaa (SEQ ID NO: 3174), UAUguaggua (SEQ ID NO: 3175), CAGguaguau (SEQ ID NO: 1225), AAGgugugcc (SEQ ID NO: 3176), UGGguaagag (SEQ ID NO: 2834), CAGgcaagca (SEQ ID NO: 3177), UUGguaaggg (SEQ ID NO: 2961), AAGgcaggug (SEQ ID NO: 109), ACGguaaaug (SEQ ID NO: 3178), GCUgugagca (SEQ ID NO: 3179), AUGguacaca (SEQ ID NO: 3180), GUAguguguu (SEQ ID NO: 3181), ACUguaagag (SEQ ID NO: 3182), CCCgcagguc (SEQ ID NO: 3183), GAGgugagcc (SEQ ID NO: 2019), GAGgugcugu (SEQ ID NO: 3184), UAAguaugcu (SEQ ID NO: 3185), GAGgccaucu (SEQ ID NO: 3186), UCAgugagug (SEQ ID NO: 2700), CAGgugcuac (SEQ ID NO: 3187), AAUgugggug (SEQ ID NO: 533), GAGgugugaa (SEQ ID NO: 3188), CUGguagguc (SEQ ID NO: 1730), GUGgcgcgcg (SEQ ID NO: 3189), CAGgugcaaa (SEQ ID NO: 1359), UAAguggagg (SEQ ID NO: 3190), CAUgugggua (SEQ ID NO: 3191), GAGguagggu (SEQ ID NO: 3192), AAAgugaguu (SEQ ID NO: 61), AGGguucuag (SEQ ID NO: 3193), UGUgugagcu (SEQ ID NO: 3194), AGGgugaauc (SEQ ID NO: 3195), CAGgucaggg (SEQ ID NO: 3196), AAGgucccug (SEQ ID NO: 3197), CUGguagagu (SEQ ID NO: 3198), UAGgucaguu (SEQ ID NO: 2570), AAAguaaggg (SEQ ID NO: 19), CAAguaugug (SEQ ID NO: 1052), CAGgugcuuu (SEQ ID NO: 3199), AAGguaauuc (SEQ ID NO: 169), GGGgugcacg (SEQ ID NO: 3200), ACUgugcuac (SEQ ID NO: 3201), CAGguaccua (SEQ ID NO: 3202), CAGguagcuu (SEQ ID NO: 1211), UGGgugaggc (SEQ ID NO: 2873), CUGguacauu (SEQ ID NO: 1718), AGGguaaucu (SEQ ID NO: 3203), CAGguacaag (SEQ ID NO: 1161), CAGguaauuc (SEQ ID NO: 1157), AGGgcacuug (SEQ ID NO: 3204), UAGgugagaa (SEQ ID NO: 2587), GAGguaaugc (SEQ ID NO: 3205), CCAgugaguu (SEQ ID NO: 3206), AAAguaugug (SEQ ID NO: 44), CUGgugaauc (SEQ ID NO: 3207), UAUguaugua (SEQ ID NO: 2663), CCUgcaggug (SEQ ID NO: 3208), CAGguaucug (SEQ ID NO: 1245), GAGgugaggu (SEQ ID NO: 3209), CUGguaaaac (SEQ ID NO: 3210), UGUgugugcu (SEQ ID NO: 3211), CAGguuaagu (SEQ ID NO: 3212), CAGguaaucc (SEQ ID NO: 1152), UAGguauuug (SEQ ID NO: 3213), UGGguagguc (SEQ ID NO: 2852), CAGguaacag (SEQ ID NO: 1129), AGCgugcgug (SEQ ID NO: 3214), AAGgucagga (SEQ ID NO: 289), GGUgugagcc (SEQ ID NO: 2312), CUGguaagua (SEQ ID NO: 1707), GGGgugggca (SEQ ID NO: 3215), AAGgugggaa (SEQ ID NO: 376), CAGgugagug (SEQ ID NO: 1347), CUGguuguua (SEQ ID NO: 3216), CAGguaauag (SEQ ID NO: 3217), UAGgugaguu (SEQ ID NO: 3218), AGAguaaguu (SEQ ID NO: 671), UAGguaaucc (SEQ ID NO: 3219), CCGgugacug (SEQ ID NO: 3220), GUCgugauua (SEQ ID NO: 3221), CUUguaagug (SEQ ID NO: 1794), UAGguaguca (SEQ ID NO: 3222), CUGguaaguc (SEQ ID NO: 3223), AGGgugagcg (SEQ ID NO: 3224), CAGguaugga (SEQ ID NO: 1255), AUUgugacca (SEQ ID NO: 3225), GUUgugggua (SEQ ID NO: 2411), AAGguacaag (SEQ ID NO: 173), CUAgcaagug (SEQ ID NO: 3226), CUGgugagau (SEQ ID NO: 3227), CAGgugggca (SEQ ID NO: 1406), AUGgcucgag (SEQ ID NO: 3228), CUGguacguu (SEQ ID NO: 1720), UUGgugugua (SEQ ID NO: 3229), GAGgugucug (SEQ ID NO: 3230), GAGgugggac (SEQ ID NO: 3231), GGGgugggag (SEQ ID NO: 3232), GCAgcgugag (SEQ ID NO: 3233), GAGguaaaga (SEQ ID NO: 1870), GAGguaugua (SEQ ID NO: 1965), AAGgugagac (SEQ ID NO: 336), AAGguacaau (SEQ ID NO: 174), CUGguaugag (SEQ ID NO: 3234), AACguaaaau (SEQ ID NO: 3235), GUGguaggga (SEQ ID NO: 2364), CUGguaugug (SEQ ID NO: 1737), CUUguaagca (SEQ ID NO: 3236), AAGguaggga (SEQ ID NO: 223), AUUguaagcc (SEQ ID NO: 3237), AUGguaagcu (SEQ ID NO: 895), CAGgugaauu (SEQ ID NO: 1322), UAGgugaaua (SEQ ID NO: 2581), CAAguaugga (SEQ ID NO: 3238), AUGguauggc (SEQ ID NO: 936), GAGgucaugc (SEQ ID NO: 3239), CAGguacccu (SEQ ID NO: 1174), ACAgugagac (SEQ ID NO: 3240), CAGgucugau (SEQ ID NO: 3241), GAAguugggu (SEQ ID NO: 3242), CUGgugegug (SEQ ID NO: 1767), CAGguacgag (SEQ ID NO: 1180), ACAgugagcc (SEQ ID NO: 556), AAGguaagua (SEQ ID NO: 153), GGAguaaggc (SEQ ID NO: 3243), GAGgugugua (SEQ ID NO: 2077), AAGgucauuu (SEQ ID NO: 3244), CAGguagucu (SEQ ID NO: 3245), AUGguaucug (SEQ ID NO: 3246), AAGguaaacu (SEQ ID NO: 125), GAGguaggug (SEQ ID NO: 1938), CUGguaagca (SEQ ID NO: 1700), AGGguaagag (SEQ ID NO: 734), AAAguaaagc (SEQ ID NO: 3247), CAGguuugag (SEQ ID NO: 1502), GAGgcgggua (SEQ ID NO: 3248), CGAguacgau (SEQ ID NO: 3249), CAGguuguug (SEQ ID NO: 1495), AAAguauggg (SEQ ID NO: 3250), UAGgcugguc (SEQ ID NO: 3251), AAGguaagga (SEQ ID NO: 149), AAGguuuccu (SEQ ID NO: 458), UUGguaaaac (SEQ ID NO: 3252), GAGguaagua (SEQ ID NO: 1893), CAGguucaag (SEQ ID NO: 1465), UGGguuaugu (SEQ ID NO: 3253), GAGgugaguu (SEQ ID NO: 2027), ACGgugaaac (SEQ ID NO: 598), GAUguaacca (SEQ ID NO: 3254), AAGgugcggg (SEQ ID NO: 3255), CCGguacgug (SEQ ID NO: 3256), GAUgugagaa (SEQ ID NO: 3257), GUGgegguga (SEQ ID NO: 3258), CAGguauuag (SEQ ID NO: 3259), GAGguuggga (SEQ ID NO: 3260), AAGgcuagua (SEQ ID NO: 3261), AAGgugggcg (SEQ ID NO: 381), CAGgcaggga (SEQ ID NO: 3262), AAUguuaguu (SEQ ID NO: 3263), GAGguaaagg (SEQ ID NO: 3264), CAGgugugcu (SEQ ID NO: 1437), CUGguaugau (SEQ ID NO: 1733), AUGguuaguc (SEQ ID NO: 978), CUGgugagaa (SEQ ID NO: 1751), CAGgccggcg (SEQ ID NO: 3265), CAGgugacug (SEQ ID NO: 1332), AAAguaaggu (SEQ ID NO: 20), UAAguacuug (SEQ ID NO: 3266), AAGguaaagc (SEQ ID NO: 127), UCGguagggg (SEQ ID NO: 3267), CAGguaggaa (SEQ ID NO: 1212), AGUguaagca (SEQ ID NO: 817), CCCgugagau (SEQ ID NO: 3268), GUGguuguuu (SEQ ID NO: 3269), CAGguuugcc (SEQ ID NO: 1504), AGGguauggg (SEQ ID NO: 766), UAAguaagug (SEQ ID NO: 2424), GAGguaagac (SEQ ID NO: 3270), GAUguagguc (SEQ ID NO: 3271), CAAguaggug (SEQ ID NO: 1043), AUAguaaaua (SEQ ID NO: 845), GAGguugggg (SEQ ID NO: 3272), GAGgcgagua (SEQ ID NO: 3273), CAGguagugu (SEQ ID NO: 1229), GUGguaggug (SEQ ID NO: 2366), CAAgugagug (SEQ ID NO: 1068), AAGgugacaa (SEQ ID NO: 330), CCAgcguaau (SEQ ID NO: 3274), ACGgugaggu (SEQ ID NO: 3275), GGGguauauu (SEQ ID NO: 3276), CAGgugagua (SEQ ID NO: 1345), AAGgugcgug (SEQ ID NO: 364), UAUguaaauu (SEQ ID NO: 3277), CAGgucagua (SEQ ID NO: 1281), ACGguacuua (SEQ ID NO: 3278), GAGgucagca (SEQ ID NO: 3279), UAAguaugua (SEQ ID NO: 2431), GGGgucagac (SEQ ID NO: 3280), AAUgugugag (SEQ ID NO: 3281), UCCgucagua (SEQ ID NO: 3282), CAGgugcuuc (SEQ ID NO: 1391), CCAguuagug (SEQ ID NO: 3283), CCGgugggcg (SEQ ID NO: 1590), AGGgugcaug (SEQ ID NO: 3284), GGGguaggau (SEQ ID NO: 3285), UAGgugggcc (SEQ ID NO: 2615), GAGguguucg (SEQ ID NO: 3286), UUGgcaagaa (SEQ ID NO: 3287), UCCguaagua (SEQ ID NO: 3288), CAGguguaag (SEQ ID NO: 3289), CUCgugagua (SEQ ID NO: 1680), GAGguguuuu (SEQ ID NO: 3290), GAGgugagca (SEQ ID NO: 2018), GAGguaaagu (SEQ ID NO: 1872), AAGguacguu (SEQ ID NO: 193), CAGguccagu (SEQ ID NO: 1291), AUGgugaaac (SEQ ID NO: 947), GUAgugagcu (SEQ ID NO: 3291), CAGgugaaaa (SEQ ID NO: 3292), AGGguacagg (SEQ ID NO: 3293), AAGguaacgc (SEQ ID NO: 3294), AAGguauacc (SEQ ID NO: 3295), CCUgugagau (SEQ ID NO: 3296), GGGguacgug (SEQ ID NO: 3297), GAGguauggu (SEQ ID NO: 1964), UAGguauuau (SEQ ID NO: 2557), GAAguaggag (SEQ ID NO: 3298), UCGguaaggg (SEQ ID NO: 3299), CCGguaagcg (SEQ ID NO: 3300), GAAguaauua (SEQ ID NO: 1823), CAGgugaguc (SEQ ID NO: 1346), AAGgucaaga (SEQ ID NO: 279), AUGguaaguc (SEQ ID NO: 899), CAGgugagcu (SEQ ID NO: 1340), CCAguuuuug (SEQ ID NO: 3301), CAGgugggag (SEQ ID NO: 1404), AAGguauuau (SEQ ID NO: 270), AAGguaaaua (SEQ ID NO: 130), AAGgugcugu (SEQ ID NO: 3302), AAAguacacc (SEQ ID NO: 3303), CUGguucgug (SEQ ID NO: 1783), UCAguaaguc (SEQ ID NO: 2690), GAAguacgug (SEQ ID NO: 3304), CAGgugacaa (SEQ ID NO: 1323), UGGguaagaa (SEQ ID NO: 2832), UGUguagggg (SEQ ID NO: 3305), GAGguaggca (SEQ ID NO: 1932), UUGgugaggc (SEQ ID NO: 3306), AUGgugugua (SEQ ID NO: 974), CAGguccucc (SEQ ID NO: 3307), UUGguaaaug (SEQ ID NO: 2953), GCUgugaguu (SEQ ID NO: 2207), AUGgucugua (SEQ ID NO: 3308), CAUgcaggug (SEQ ID NO: 3309), CUGguacacc (SEQ ID NO: 3310), CAGguccuua (SEQ ID NO: 3311), CAAguaaucu (SEQ ID NO: 1031), AUGgcagccu (SEQ ID NO: 3312), AAGgucagaa (SEQ ID NO: 282), AACgugaggc (SEQ ID NO: 3313), CAGgcacgca (SEQ ID NO: 1106), ACGguccagg (SEQ ID NO: 3314), UCUguacaua (SEQ ID NO: 3315), GAGgugauua (SEQ ID NO: 3316), ACGguaaaua (SEQ ID NO: 3317), AUGguaacug (SEQ ID NO: 3318), CAGgcgcguu (SEQ ID NO: 3319), CAGguauaga (SEQ ID NO: 1235), AAGguuuguu (SEQ ID NO: 3320), CAGguaugaa (SEQ ID NO: 1247), UAGguuggua (SEQ ID NO: 2638), CUGgugagac (SEQ ID NO: 1752), CAGguuagga (SEQ ID NO: 3321), AUGgugacug (SEQ ID NO: 3322), UUGguauccc (SEQ ID NO: 3323), CUUguaggac (SEQ ID NO: 3324), AAAguguguu (SEQ ID NO: 69), CAGguuucuu (SEQ ID NO: 1500), GGGguauggc (SEQ ID NO: 3325), GGGguaggac (SEQ ID NO: 3326), ACUguaaguc (SEQ ID NO: 626), AUCguaagcu (SEQ ID NO: 3327), UAGguucccc (SEQ ID NO: 2636), GGUgugagca (SEQ ID NO: 3328), CUGguuggua (SEQ ID NO: 3329), GGGguuaggg (SEQ ID NO: 3330), UGAguaagaa (SEQ ID NO: 3331), GAGguauucc (SEQ ID NO: 1969), UGGguuaguc (SEQ ID NO: 2893), CAGgcucgug (SEQ ID NO: 3332), UAGguagagu (SEQ ID NO: 3333), UAGgugcccu (SEQ ID NO: 3334), AAAgugagua (SEQ ID NO: 58), GAGguucaua (SEQ ID NO: 2094), UUGguaagag (SEQ ID NO: 2958), ACCgugugua (SEQ ID NO: 3335), UAUguaguau (SEQ ID NO: 3336), UGGguaauag (SEQ ID NO: 3337), CAGgucugaa (SEQ ID NO: 3338), AAAguauaaa (SEQ ID NO: 3339), GUGgugaguc (SEQ ID NO: 3340), AGUgugauua (SEQ ID NO: 3341), UUGgugugug (SEQ ID NO: 3020), CAGgugaugg (SEQ ID NO: 1353), GCUgugagua (SEQ ID NO: 2204), CAGguacaug (SEQ ID NO: 1169), AAGguacagu (SEQ ID NO: 178), GAAguuguag (SEQ ID NO: 3342), CAGgugauua (SEQ ID NO: 1355), UAGgugaauu (SEQ ID NO: 2583), GGUguuaaua (SEQ ID NO: 3343), CAGguauuua (SEQ ID NO: 1268), CAAguacucg (SEQ ID NO: 3344), CAAguaagaa (SEQ ID NO: 1022), AAGguaccuu (SEQ ID NO: 188), ACGgugaggg (SEQ ID NO: 3345), UGAgcaggca (SEQ ID NO: 3346), GGGgugaccg (SEQ ID NO: 3347), GAGguaaaug (SEQ ID NO: 1875), CGGguuugug (SEQ ID NO: 3348), AAGgugagcg (SEQ ID NO: 341), GUGguaugga (SEQ ID NO: 3349), CUGguaagga (SEQ ID NO: 1703), GAGguaccag (SEQ ID NO: 1911), CCGgugagug (SEQ ID NO: 1587), AAGguuagaa (SEQ ID NO: 416), GAGguacuug (SEQ ID NO: 1921), AGAguaaaac (SEQ ID NO: 651), UCUgugagua (SEQ ID NO: 2760), AAGgcgggaa (SEQ ID NO: 3350), CAGguaugcg (SEQ ID NO: 1253), AGGguaaaac (SEQ ID NO: 3351), AAGgugacug (SEQ ID NO: 333), AGGguauguu (SEQ ID NO: 3352), AAGguaugua (SEQ ID NO: 263), CAGgucucuc (SEQ ID NO: 1302), CAGgcaugua (SEQ ID NO: 3353), CUGguaggua (SEQ ID NO: 1729), AAGgucaugc (SEQ ID NO: 3354), CAGguacaca (SEQ ID NO: 1163), GAUguacguu (SEQ ID NO: 3355), ACAguacgug (SEQ ID NO: 3356), ACGguaccca (SEQ ID NO: 3357), CAGguagugc (SEQ ID NO: 3358), ACAguaagag (SEQ ID NO: 3359), GGUgcacacc (SEQ ID NO: 3360), GAGguguaac (SEQ ID NO: 3361), AAGgugugua (SEQ ID NO: 403), UAGguacuua (SEQ ID NO: 3362), GCGguacugc (SEQ ID NO: 3363), UGGguaaguc (SEQ ID NO: 2842), CAUguaggua (SEQ ID NO: 1529), CAGguaggau (SEQ ID NO: 3364), CAGgucuggc (SEQ ID NO: 3365), GUGguuuuaa (SEQ ID NO: 3366), CAGgugggaa (SEQ ID NO: 1402), UGGgugagua (SEQ ID NO: 2875), CGAgugagcc (SEQ ID NO: 3367), AAGguauggc (SEQ ID NO: 261), AGUguuguca (SEQ ID NO: 3368), CAGgugauuu (SEQ ID NO: 1358), UAGguaucuc (SEQ ID NO: 2544), UAAguauguu (SEQ ID NO: 3369), AAGguugagc (SEQ ID NO: 3370), AGAguaaaga (SEQ ID NO: 653), GGUguaagua (SEQ ID NO: 3371), GGGgugagcu (SEQ ID NO: 2279), CAGguauaau (SEQ ID NO: 3372), GAGguacaaa (SEQ ID NO: 1904), AUGguaccaa (SEQ ID NO: 3373), UAGguagggg (SEQ ID NO: 2523), UGAgucagaa (SEQ ID NO: 3374), AAGgcaauua (SEQ ID NO: 3375), UUGguaagau (SEQ ID NO: 3376), CAGguacaga (SEQ ID NO: 1165), AGAguuagag (SEQ ID NO: 3377), CAGgugcguc (SEQ ID NO: 1381), GAGguauuac (SEQ ID NO: 3378), ACGguacaga (SEQ ID NO: 3379), CAGgucuucc (SEQ ID NO: 1313), AAGguaaggu (SEQ ID NO: 152), GAGguaauuu (SEQ ID NO: 1903), AGUguaggcu (SEQ ID NO: 3380), AAAguaagcg (SEQ ID NO: 3381), CCUguaagcc (SEQ ID NO: 3382), AGGgugauuu (SEQ ID NO: 3383), UGUguaugaa (SEQ ID NO: 3384), CUGguacaca (SEQ ID NO: 3385), AGGguagaga (SEQ ID NO: 3386), AUAguaagca (SEQ ID NO: 848), AGAguaugua (SEQ ID NO: 3387), UUGgucagca (SEQ ID NO: 3388), CAGgcaaguu (SEQ ID NO: 1105), AAGguauaua (SEQ ID NO: 242), AAGgucugga (SEQ ID NO: 314), CAGguacgca (SEQ ID NO: 1181), AGGgugcggg (SEQ ID NO: 3389), AUGguaagug (SEQ ID NO: 900), AAAgugauga (SEQ ID NO: 3390), UGCgugagua (SEQ ID NO: 3391), AGAguaggga (SEQ ID NO: 684), UGUguaggua (SEQ ID NO: 2912), UAGguaggau (SEQ ID NO: 2521), UAAgugagug (SEQ ID NO: 2440), GCUguaagua (SEQ ID NO: 2193), GAAguaagaa (SEQ ID NO: 1814), UCGgugaggc (SEQ ID NO: 2733), UAGguauuuu (SEQ ID NO: 2564), AAGguacaca (SEQ ID NO: 3392), AAGguaggua (SEQ ID NO: 227), UGGguagguu (SEQ ID NO: 2854), ACAgcaagua (SEQ ID NO: 541), GAGguaggag (SEQ ID NO: 1931), UGGgugaguu (SEQ ID NO: 2878), GCGgugagau (SEQ ID NO: 3393), CCUguagguu (SEQ ID NO: 3394), CAGgugugua (SEQ ID NO: 1440), CUGguaagcc (SEQ ID NO: 1701), AAGgugauuc (SEQ ID NO: 3395), CAGguagcua (SEQ ID NO: 1208), GUUguaagug (SEQ ID NO: 3396), AUGguaagca (SEQ ID NO: 893), AUAguaggga (SEQ ID NO: 3397), GGGguucgcu (SEQ ID NO: 3398), CCGgucagag (SEQ ID NO: 3399), GUAguaugag (SEQ ID NO: 3400), CGUguaagau (SEQ ID NO: 3401), UGAguaggca (SEQ ID NO: 3402), UCAguaugua (SEQ ID NO: 3403), GAGguaucug (SEQ ID NO: 1954), AGAguauuuu (SEQ ID NO: 3404), AAGguuguag (SEQ ID NO: 3405), AGUguaaguu (SEQ ID NO: 821), CGGguaaguu (SEQ ID NO: 1626), UCGgugcgga (SEQ ID NO: 3406), UAGguaagua (SEQ ID NO: 2491), GAAguuagau (SEQ ID NO: 3407), GCUgugagac (SEQ ID NO: 3408), CAGgcaggua (SEQ ID NO: 3409), CAGguagggg (SEQ ID NO: 1218), UAAguuaaga (SEQ ID NO: 3410), AUGguggguu (SEQ ID NO: 970), UAGguaaguu (SEQ ID NO: 2494), CUGguaaauu (SEQ ID NO: 1690), CCGguaagga (SEQ ID NO: 1577), GAGgcaggca (SEQ ID NO: 3411), CAUguaagug (SEQ ID NO: 1523), AAGgugccua (SEQ ID NO: 3412), UUGguaggga (SEQ ID NO: 2977), AAGguaaaca (SEQ ID NO: 123), CGGgugugag (SEQ ID NO: 3413), GGGgugugag (SEQ ID NO: 3414), UCCguggguc (SEQ ID NO: 3415), ACGguaaauc (SEQ ID NO: 3416), UCAguaggua (SEQ ID NO: 3417), CAGgucagcc (SEQ ID NO: 1278), CAGgcggugg (SEQ ID NO: 3418), CGAguaagcu (SEQ ID NO: 3419), CCCgugagca (SEQ ID NO: 3420), AAAguaauga (SEQ ID NO: 3421), CUGguaagcu (SEQ ID NO: 1702), CGGguaacca (SEQ ID NO: 3422), CAGgucgcac (SEQ ID NO: 3423), GAGguaggcc (SEQ ID NO: 3424), UAGgugagcc (SEQ ID NO: 2591), UAGguaggca (SEQ ID NO: 3425), GCGgugcgug (SEQ ID NO: 3426), AUGgugagua (SEQ ID NO: 961), GGGgugaggg (SEQ ID NO: 2282), GAGgucacac (SEQ ID NO: 3427), CAGguaggcc (SEQ ID NO: 3428), CAAgugcuga (SEQ ID NO: 3429), GUCgucuuca (SEQ ID NO: 3430), CAUguaagaa (SEQ ID NO: 1518), GUAguaagga (SEQ ID NO: 3431), UAGguuugua (SEQ ID NO: 2643), CAAguuagag (SEQ ID NO: 3432), AAGguagagu (SEQ ID NO: 208), AAGgugagau (SEQ ID NO: 338), AAAguaggua (SEQ ID NO: 37), ACAgugaauc (SEQ ID NO: 3433), CAGgugugcg (SEQ ID NO: 1436), CAGgucggcc (SEQ ID NO: 1299), AAGguaguau (SEQ ID NO: 3434), ACUgucaguc (SEQ ID NO: 3435), UCUgcagccu (SEQ ID NO: 3436), CGAguaagug (SEQ ID NO: 3437), AGAguaauua (SEQ ID NO: 3438), AGUgugagug (SEQ ID NO: 837), CCGgugagcg (SEQ ID NO: 3439), AAGguaaccu (SEQ ID NO: 3440), AAGguugugg (SEQ ID NO: 3441), AAGgcauggg (SEQ ID NO: 3442), AAGgucagag (SEQ ID NO: 284), ACGguaaggu (SEQ ID NO: 3443), GGGgugagca (SEQ ID NO: 3444), GAGguugcuu (SEQ ID NO: 3445), AAGguaucgc (SEQ ID NO: 3446), CCGguaaagg (SEQ ID NO: 3447), AAAguuaaug (SEQ ID NO: 3448), UAGguacgag (SEQ ID NO: 2510), ACCguaauua (SEQ ID NO: 3449), GGGguaagga (SEQ ID NO: 2249), CCGguaacgc (SEQ ID NO: 3450), CAGgucagaa (SEQ ID NO: 1275), AAGguacuga (SEQ ID NO: 197), GAGgugacca (SEQ ID NO: 2010), GGGgugagcc (SEQ ID NO: 2277), AAGguacagg (SEQ ID NO: 177), AUGguaauua (SEQ ID NO: 3451), CAGgugagag (SEQ ID NO: 1335), AAGgugacuc (SEQ ID NO: 3452), AUAguaagua (SEQ ID NO: 849), GAGguaaacc (SEQ ID NO: 1869), CAGgugggau (SEQ ID NO: 1405), CAGgugagaa (SEQ ID NO: 1333), AGGguaaaaa (SEQ ID NO: 3453), GAGgugugac (SEQ ID NO: 3454), CACguaagcu (SEQ ID NO: 3455), CAGguccccc (SEQ ID NO: 3456), CAGgucaggu (SEQ ID NO: 3457), CGGguaaguc (SEQ ID NO: 3458), ACGguauggg (SEQ ID NO: 3459), GAUguaaguu (SEQ ID NO: 2123), CAAguaauau (SEQ ID NO: 3460), CAGguugggg (SEQ ID NO: 3461), CCUgugcugg (SEQ ID NO: 3462), AAGguaugau (SEQ ID NO: 256), AGGguagagg (SEQ ID NO: 3463), AAGguggguu (SEQ ID NO: 386), CAGgugugaa (SEQ ID NO: 1430), UUGguaugug (SEQ ID NO: 2988), UUGguaucuc (SEQ ID NO: 2985), GGGgugagug (SEQ ID NO: 2284), CUGgugugug (SEQ ID NO: 1779), AGGguagggc (SEQ ID NO: 3464), GUGgugagua (SEQ ID NO: 3465), CAGguaugua (SEQ ID NO: 1258), AAGguacauu (SEQ ID NO: 181), UUAguaagug (SEQ ID NO: 2934), AAUguauauc (SEQ ID NO: 3466), CUUguaagua (SEQ ID NO: 1793), GAGguuagua (SEQ ID NO: 2087), CAGguaaggu (SEQ ID NO: 1146), CAGguaaugu (SEQ ID NO: 1155), AGGgugaggc (SEQ ID NO: 3467), CAGguauuuc (SEQ ID NO: 1269), CAGgucugga (SEQ ID NO: 1307), GGGgugugcu (SEQ ID NO: 3468), UAGgugagug (SEQ ID NO: 2598), AAUguaaccu (SEQ ID NO: 3469), UAAgugaguc (SEQ ID NO: 2439), CAGgugcacu (SEQ ID NO: 3470), ACGguaagua (SEQ ID NO: 579), GAGguauccu (SEQ ID NO: 3471), UCUguaaguc (SEQ ID NO: 2745), CAGguauuca (SEQ ID NO: 1263), UGUguaagug (SEQ ID NO: 3472), CCAgcaaggc (SEQ ID NO: 3473), GAGgugaagg (SEQ ID NO: 2006), AAUguggggu (SEQ ID NO: 3474), UCGgugcgug (SEQ ID NO: 3475), UUGguaaggc (SEQ ID NO: 3476), GAGguaagug (SEQ ID NO: 3477), AAAguaagau (SEQ ID NO: 14), UAGgucuuuu (SEQ ID NO: 3478), GAGgucugau (SEQ ID NO: 3479), CCAguuagag (SEQ ID NO: 3480), UGGgugaaaa (SEQ ID NO: 3481), AGAguaagau (SEQ ID NO: 662), CAGguaauug (SEQ ID NO: 1158), CAGgccgguc (SEQ ID NO: 3482), CCGguaagag (SEQ ID NO: 3483), GAGgugagcu (SEQ ID NO: 2021), CUGguaagac (SEQ ID NO: 3484), CAGgugagau (SEQ ID NO: 1336), CUGguuuguu (SEQ ID NO: 3485), UGGguaggua (SEQ ID NO: 3486), CAGguuagug (SEQ ID NO: 1457), CAGguguucg (SEQ ID NO: 3487), CGGguagguc (SEQ ID NO: 3488), GUGguacaua (SEQ ID NO: 3489), AAGguacuaa (SEQ ID NO: 194), GAUgugagua (SEQ ID NO: 3490), UGUguaagac (SEQ ID NO: 2904), GAGguagccg (SEQ ID NO: 3491), UAGgugaucu (SEQ ID NO: 3492), CAGguacgug (SEQ ID NO: 1185), CUUgucaguc (SEQ ID NO: 3493), GAGguaucac (SEQ ID NO: 3494), GAGguaauga (SEQ ID NO: 3495), AAGguaacac (SEQ ID NO: 3496), CAGguaaagc (SEQ ID NO: 1123), AAGgcaagua (SEQ ID NO: 3497), CGCgugagcc (SEQ ID NO: 3498), AGUgugcguu (SEQ ID NO: 3499), GAUguaagca (SEQ ID NO: 2118), AAGguaauag (SEQ ID NO: 159), GGAgcaguug (SEQ ID NO: 3500), AGCguaagau (SEQ ID NO: 3501), AAGgucaggc (SEQ ID NO: 290), GAGguauuca (SEQ ID NO: 3502), AAUguaaagu (SEQ ID NO: 3503), CAGguaacaa (SEQ ID NO: 3504), UCGguaggug (SEQ ID NO: 3505), AAAguaaguc (SEQ ID NO: 22), CGGgugcagu (SEQ ID NO: 3506), GGUgugugca (SEQ ID NO: 3507), UGAgugagaa (SEQ ID NO: 2794), CACguguaag (SEQ ID NO: 3508), GUGguuggua (SEQ ID NO: 3509), GCAgccuuga (SEQ ID NO: 3510), CGAgugugau (SEQ ID NO: 3511), CAGguauaua (SEQ ID NO: 3512), UAUguaugug (SEQ ID NO: 2665), CCCgugguca (SEQ ID NO: 3513), AUGguaagac (SEQ ID NO: 890), GAGgugugga (SEQ ID NO: 2074), AGUguauccu (SEQ ID NO: 3514), UGAguguguc (SEQ ID NO: 3515), UGGguaaucu (SEQ ID NO: 3516), AUGgcagguu (SEQ ID NO: 3517), GAGguaagau (SEQ ID NO: 1884), UCAgcagcgu (SEQ ID NO: 3518), AAGgugggau (SEQ ID NO: 378), CGGgugcgcu (SEQ ID NO: 3519), CAGgugucug (SEQ ID NO: 1429), AGCgugguaa (SEQ ID NO: 3520), AAUgugaaug (SEQ ID NO: 3521), UCGgugagac (SEQ ID NO: 3522), UAGguaaagc (SEQ ID NO: 3523), CUGguaaaag (SEQ ID NO: 3524), CCGgugcgga (SEQ ID NO: 3525), CAGguacuca (SEQ ID NO: 3526), CAGguagcaa (SEQ ID NO: 1203), GAAguugagu (SEQ ID NO: 3527), GAGguggagg (SEQ ID NO: 2052), AGGguaugag (SEQ ID NO: 762), UAGguaugcu (SEQ ID NO: 3528), UAGgugagac (SEQ ID NO: 2588), CAGguaauua (SEQ ID NO: 1156), CGUguaagcc (SEQ ID NO: 3529), CUUguaaguu (SEQ ID NO: 1795), AAGguaacuu (SEQ ID NO: 140), UCGgcaaggc (SEQ ID NO: 3530), GAGguucucg (SEQ ID NO: 3531), GAGgugggcg (SEQ ID NO: 2058), AAGgcaugug (SEQ ID NO: 3532), CUGguauguu (SEQ ID NO: 1738), UAAgucauuu (SEQ ID NO: 3533), CAUguaauua (SEQ ID NO: 1525), AAUguaaaga (SEQ ID NO: 3534), UAGgugcuca (SEQ ID NO: 3535), AAGguaaugg (SEQ ID NO: 166), GAGguacuga (SEQ ID NO: 3536), UGGguaagua (SEQ ID NO: 2841), UGGguaaaaa (SEQ ID NO: 3537), AAGgugagcu (SEQ ID NO: 342), UACgugaguu (SEQ ID NO: 3538), AGGgugagcc (SEQ ID NO: 790), CGGgugagga (SEQ ID NO: 3539), UGGgugagag (SEQ ID NO: 2869), GGUguaagcu (SEQ ID NO: 3540), CGGguggguu (SEQ ID NO: 1648), CCAgcuaagu (SEQ ID NO: 3541), AAGguuuguc (SEQ ID NO: 467), GAGguuagac (SEQ ID NO: 2084), GAGguaccuc (SEQ ID NO: 3542), UUUguaaguu (SEQ ID NO: 3041), GAGguuagga (SEQ ID NO: 3543), CAGguaggga (SEQ ID NO: 1216), AGGguaauac (SEQ ID NO: 744), UGCgugugua (SEQ ID NO: 3544), CCAguaacca (SEQ ID NO: 3545), AGGgucuguc (SEQ ID NO: 3546), UGGguaugua (SEQ ID NO: 2860), GUGguaagcu (SEQ ID NO: 2348), CAGguaaccu (SEQ ID NO: 3547), AAGgugaguu (SEQ ID NO: 350), UAGguucgug (SEQ ID NO: 3548), AAAguuagua (SEQ ID NO: 3549), UGGgcaaguc (SEQ ID NO: 2816), AAGgcacagu (SEQ ID NO: 3550), GUUguaaguc (SEQ ID NO: 2401), AAGguuugcc (SEQ ID NO: 462), CUUgcauggg (SEQ ID NO: 3551), GCGgugagua (SEQ ID NO: 3552), GGGguaagcg (SEQ ID NO: 3553), GCCguaagaa (SEQ ID NO: 3554), GAGgucggga (SEQ ID NO: 3555), UUGguauugu (SEQ ID NO: 2990), AGUgugagac (SEQ ID NO: 3556), CUGgugggga (SEQ ID NO: 1770), AGAguaaggu (SEQ ID NO: 668), CCGguggguc (SEQ ID NO: 3557), CAGguauucu (SEQ ID NO: 1264), UGGguaacgu (SEQ ID NO: 3558), UUGgugagag (SEQ ID NO: 3559), UAGguacccu (SEQ ID NO: 3560), GGGgugcguc (SEQ ID NO: 3561), AAGgcaggag (SEQ ID NO: 3562), ACGguacauu (SEQ ID NO: 3563), GAGguaguua (SEQ ID NO: 1946), CAGguauggg (SEQ ID NO: 1256), UUUguguguc (SEQ ID NO: 3053), CAGguacuua (SEQ ID NO: 1194), AUGguauacu (SEQ ID NO: 3564), AGUgugagcc (SEQ ID NO: 833), ACAguaacga (SEQ ID NO: 3565), CUGguaccca (SEQ ID NO: 3566), CAGguaaccc (SEQ ID NO: 3567), GGAguaagua (SEQ ID NO: 3568), GAGgugggug (SEQ ID NO: 2065), ACUguauguc (SEQ ID NO: 3569), ACGgugagua (SEQ ID NO: 606), CUGguaaugu (SEQ ID NO: 3570), AAGguaucag (SEQ ID NO: 247), CAGgugcccc (SEQ ID NO: 1370), AGUgucagug (SEQ ID NO: 3571), AAGguaggag (SEQ ID NO: 218), GGAguaugug (SEQ ID NO: 3572), UUGguauuuu (SEQ ID NO: 2992), CCUguuguga (SEQ ID NO: 3573), UUUguaagaa (SEQ ID NO: 3033), UAGguaacau (SEQ ID NO: 2475), CAGguaagca (SEQ ID NO: 3574), CAGgucacag (SEQ ID NO: 3575), CAGgugugag (SEQ ID NO: 1432), UAGguuugcg (SEQ ID NO: 3576), CUGguaagaa (SEQ ID NO: 1697), ACGguuguau (SEQ ID NO: 3577), AAGguugggg (SEQ ID NO: 446), AAGgugaauu (SEQ ID NO: 329), GGGguuaguu (SEQ ID NO: 3578), ACGguaaggc (SEQ ID NO: 3579), CAGguuuaag (SEQ ID NO: 1496), CUGguaaguu (SEQ ID NO: 1709), GGGgugagag (SEQ ID NO: 3580), UGGguggguu (SEQ ID NO: 2886), GAGguuuguu (SEQ ID NO: 2111), UGGguaaaug (SEQ ID NO: 2826), CAGgcaggcc (SEQ ID NO: 3581), CACgugcagg (SEQ ID NO: 3582), AAGgugagcc (SEQ ID NO: 340), CAAguaagug (SEQ ID NO: 1028), CAGgucaguc (SEQ ID NO: 1282), GCGguauaau (SEQ ID NO: 3583), UAGguaaagu (SEQ ID NO: 3584), UAGguggauu (SEQ ID NO: 3585), GAGgucugga (SEQ ID NO: 3586), UCGgucaguu (SEQ ID NO: 3587), UGGguaacug (SEQ ID NO: 3588), AAGguuugau (SEQ ID NO: 3589), UGUgcuggug (SEQ ID NO: 3590), UGUguaccuc (SEQ ID NO: 3591), UGGguacagu (SEQ ID NO: 2849), AUCgucagcg (SEQ ID NO: 3592), CAGgucuugg (SEQ ID NO: 3593), GAAguuggua (SEQ ID NO: 3594), GAAguaaaga (SEQ ID NO: 3595), UUGguaagcu (SEQ ID NO: 2959), UAGguaccag (SEQ ID NO: 2507), AGGguaucau (SEQ ID NO: 3596), CAGguaaaaa (SEQ ID NO: 1118), ACGguaauuu (SEQ ID NO: 583), AUUguaaguu (SEQ ID NO: 997), GAGguacagu (SEQ ID NO: 1908), CAGgugaaag (SEQ ID NO: 1315), UGGguuguuu (SEQ ID NO: 3597), GGGguaggug (SEQ ID NO: 2259), CAGgugccca (SEQ ID NO: 1369), AGCgugagau (SEQ ID NO: 3598), CCAgugagug (SEQ ID NO: 1565), AGGguagaug (SEQ ID NO: 3599), UGGguguguc (SEQ ID NO: 2888), AUCgcgugag (SEQ ID NO: 3600), AGGguaagcc (SEQ ID NO: 3601), AGGguagcag (SEQ ID NO: 3602), UUCguuuccg (SEQ ID NO: 3603), AAGguaagcg (SEQ ID NO: 147), UGGguaagcc (SEQ ID NO: 2837), CAGguauggc (SEQ ID NO: 3604), UGUguaagua (SEQ ID NO: 2907), AAGguagaga (SEQ ID NO: 3605), ACGguaauaa (SEQ ID NO: 3606), CUGguacggu (SEQ ID NO: 3607), GAGgucacag (SEQ ID NO: 3608), UAUguaaguu (SEQ ID NO: 2656), CUGguacgcc (SEQ ID NO: 3609), CAAguaagau (SEQ ID NO: 1024), CUAgugagua (SEQ ID NO: 1673), CCGguaaccg (SEQ ID NO: 3610), CUUguaaguc (SEQ ID NO: 3611), GUGgugagaa (SEQ ID NO: 2378), ACCguaugua (SEQ ID NO: 3612), GUAguaagug (SEQ ID NO: 2324), UUGgugggua (SEQ ID NO: 3014), CGGguacuuu (SEQ ID NO: 3613), UGGguaaaua (SEQ ID NO: 2825), AGAgugagua (SEQ ID NO: 704), AAGguagguu (SEQ ID NO: 230), AAGguaugcg (SEQ ID NO: 3614), CCUguaggcu (SEQ ID NO: 3615), ACAguagaaa (SEQ ID NO: 3616), CCGguuagua (SEQ ID NO: 3617), CGGguaggcg (SEQ ID NO: 3618), GCAgugagug (SEQ ID NO: 2162), GAGgugaguc (SEQ ID NO: 3619), CUGguagccu (SEQ ID NO: 3620), CAUguaugua (SEQ ID NO: 1533), GAAguaacuu (SEQ ID NO: 3621), GAAguaagau (SEQ ID NO: 3622), AAGguuagau (SEQ ID NO: 417), AAGguaauca (SEQ ID NO: 161), AAUguaugua (SEQ ID NO: 507), UGAguaagau (SEQ ID NO: 2767), AGAgugagca (SEQ ID NO: 703), GUAguucuau (SEQ ID NO: 3623), GAGguaauca (SEQ ID NO: 1898), UAGguaugga (SEQ ID NO: 2548), UAGgugggac (SEQ ID NO: 2612), GAGguacaug (SEQ ID NO: 3624), UGGguaaggc (SEQ ID NO: 3625), CAGguacgcc (SEQ ID NO: 1182), CCAguuacgc (SEQ ID NO: 3626), ACUgugguga (SEQ ID NO: 3627), GAGguaaguc (SEQ ID NO: 1894), AUUguaggug (SEQ ID NO: 3628), ACCgucagug (SEQ ID NO: 3629), AAUgugaggg (SEQ ID NO: 3630), ACUgugagug (SEQ ID NO: 645), UGGguguggu (SEQ ID NO: 3631), AAGguuggga (SEQ ID NO: 445), AAGguuugga (SEQ ID NO: 464), UCCgugagug (SEQ ID NO: 3632), CGGgugagug (SEQ ID NO: 1642), AGAguaagcu (SEQ ID NO: 664), CAGgcaagcu (SEQ ID NO: 3633), UAGguauauu (SEQ ID NO: 2541), AAAguagcag (SEQ ID NO: 3634), GAGguaaccu (SEQ ID NO: 1880), AAGgugggca (SEQ ID NO: 379), AGGgugagua (SEQ ID NO: 795), UGGguaaggu (SEQ ID NO: 2840), CUUgucagug (SEQ ID NO: 3635), UAGgugcgcu (SEQ ID NO: 3636), GAGgcaaauu (SEQ ID NO: 3637), AGGguaccuc (SEQ ID NO: 3638), CAAgugcgua (SEQ ID NO: 3639), AGAguaagac (SEQ ID NO: 660), GUGguaaaua (SEQ ID NO: 3640), GAUguaagcg (SEQ ID NO: 3641), GAGguaaagc (SEQ ID NO: 1871), UAGgugagua (SEQ ID NO: 2596), CAGguaacau (SEQ ID NO: 1130), CCUguacggc (SEQ ID NO: 3642), UAGguauguc (SEQ ID NO: 2552), UAGguccaua (SEQ ID NO: 3643), GAGgugaaaa (SEQ ID NO: 2003), AAAguacuga (SEQ ID NO: 3644), UUGguaagcg (SEQ ID NO: 3645), CAGgcaagcg (SEQ ID NO: 3646), UUUgcagguu (SEQ ID NO: 3647), CAGguuuaua (SEQ ID NO: 3648), CUGguaaagc (SEQ ID NO: 1686), AUGgugagcu (SEQ ID NO: 958), CAGgugguug (SEQ ID NO: 1419), GUAguaaguu (SEQ ID NO: 3649), CAGguaauac (SEQ ID NO: 3650), CAGgcaaggc (SEQ ID NO: 3651), AAGguaauuu (SEQ ID NO: 171), UUUguccgug (SEQ ID NO: 3652), GAGguagguu (SEQ ID NO: 1939), ACCgugagug (SEQ ID NO: 3653), CAAguaagcu (SEQ ID NO: 3654), ACAgugagua (SEQ ID NO: 560), UUGgugagau (SEQ ID NO: 3000), AAGguagucu (SEQ ID NO: 233), CAGguaaagg (SEQ ID NO: 3655), GGGguaugga (SEQ ID NO: 2264), UUUguaagug (SEQ ID NO: 3040), GUGguaagag (SEQ ID NO: 2344), AGUgugaguu (SEQ ID NO: 838), AAGgcaagcg (SEQ ID NO: 3656), UAAgugagua (SEQ ID NO: 2438), AGGgugagug (SEQ ID NO: 797), AGUguacgug (SEQ ID NO: 3657), AGGgugcgua (SEQ ID NO: 3658), GGCgugagcc (SEQ ID NO: 2238), CGAguuauga (SEQ ID NO: 3659), CAGguaaaga (SEQ ID NO: 1122), UUGgugaaga (SEQ ID NO: 3660), AGGguaaugg (SEQ ID NO: 3661), AAGguccaga (SEQ ID NO: 300), AGUgugaguc (SEQ ID NO: 836), CAGguaauuu (SEQ ID NO: 1159), CAGguaacgc (SEQ ID NO: 3662), CUGguacacu (SEQ ID NO: 3663), CUGguuagug (SEQ ID NO: 1782), CAGguacuug (SEQ ID NO: 3664), CACguaagua (SEQ ID NO: 3665), GUGgugegge (SEQ ID NO: 3666), GAGgucaguu (SEQ ID NO: 3667), AUGguaugcc (SEQ ID NO: 932), AAGgugugug (SEQ ID NO: 405), CUGguggguc (SEQ ID NO: 1772), CAGgugaggc (SEQ ID NO: 1342), AAGguuaguc (SEQ ID NO: 423), AAGguagcug (SEQ ID NO: 215), GAGgucagga (SEQ ID NO: 1983), GUUguaggua (SEQ ID NO: 3668), UGGguacaag (SEQ ID NO: 3669), AUGguaggug (SEQ ID NO: 924), GAGguaagcc (SEQ ID NO: 1886), AUGgcaagua (SEQ ID NO: 3670), AAGguauauu (SEQ ID NO: 245), GCGgugagag (SEQ ID NO: 3671), AAGgugcuuc (SEQ ID NO: 3672), UAGguacauc (SEQ ID NO: 3673), ACUgugguaa (SEQ ID NO: 3674), GAGguaggcu (SEQ ID NO: 1933), GAGguaugca (SEQ ID NO: 3675), AGGguaguuc (SEQ ID NO: 3676), CAGguauccu (SEQ ID NO: 1241), AGGguaaguc (SEQ ID NO: 741), AGGgucaguu (SEQ ID NO: 779), CAGguuggga (SEQ ID NO: 3677), CAGguggaua (SEQ ID NO: 3678), GGAguagguu (SEQ ID NO: 2220), GAGguaggau (SEQ ID NO: 3679), GGGguuugug (SEQ ID NO: 3680), UAGguaauug (SEQ ID NO: 3681), AAGguaaccc (SEQ ID NO: 136), ACGguaagaa (SEQ ID NO: 3682), GAGguagggg (SEQ ID NO: 1936), CGAguaggug (SEQ ID NO: 1619), UCCguaagug (SEQ ID NO: 2710), UCGguacagg (SEQ ID NO: 3683), CAAguaagcg (SEQ ID NO: 3684), AAGguccgcg (SEQ ID NO: 3685), AAUgugagua (SEQ ID NO: 523), CAGgugaaug (SEQ ID NO: 3686), GUGguaaggc (SEQ ID NO: 2350), AGAgugagug (SEQ ID NO: 706), UCUguauguc (SEQ ID NO: 3687), UGGgugaguc (SEQ ID NO: 2876), UCGguuagua (SEQ ID NO: 3688), GAUguaugca (SEQ ID NO: 3689), GAGguuggug (SEQ ID NO: 3690), GAGguggggc (SEQ ID NO: 2061), UGGgucaguc (SEQ ID NO: 3691), GCAgugagua (SEQ ID NO: 2161), CAGguugcuu (SEQ ID NO: 3692), AGGguagagu (SEQ ID NO: 3693), UAGgucaggu (SEQ ID NO: 2567), CGCguaugua (SEQ ID NO: 3694), GAGguauuaa (SEQ ID NO: 3695), CAGguaaacu (SEQ ID NO: 3696), AAAguaaguu (SEQ ID NO: 24), GGGgucuggc (SEQ ID NO: 3697), GCUguggggu (SEQ ID NO: 3698), UUGguaaguc (SEQ ID NO: 3699), AAGguagaag (SEQ ID NO: 3700), AAUgugaguc (SEQ ID NO: 524), AAGgucagcu (SEQ ID NO: 288), AAGguaagag (SEQ ID NO: 143), AUGgugagga (SEQ ID NO: 3701), AAGguacuuc (SEQ ID NO: 200), AAGguaagaa (SEQ ID NO: 141), CCGguacagc (SEQ ID NO: 3702), GCGgugcgga (SEQ ID NO: 3703), CAGguacaua (SEQ ID NO: 1168), CUGgugagga (SEQ ID NO: 1755), CUGguaggug (SEQ ID NO: 1731), AACguagguu (SEQ ID NO: 3704), AUGgugugug (SEQ ID NO: 975), UUGguacuau (SEQ ID NO: 3705), CAGgucggug (SEQ ID NO: 1300), CAGgcauggg (SEQ ID NO: 3706), AUGguaucuu (SEQ ID NO: 929), AAGguaacua (SEQ ID NO: 137), CAGgugggcg (SEQ ID NO: 3707), CACgugagga (SEQ ID NO: 3708), AAGgugguuc (SEQ ID NO: 392), UGGgcauucu (SEQ ID NO: 3709), AUGguaagcc (SEQ ID NO: 894), AGGgucagug (SEQ ID NO: 778), AGAguacgua (SEQ ID NO: 3710), AAGguaggca (SEQ ID NO: 220), AAGguauuca (SEQ ID NO: 3711), CAGguagauu (SEQ ID NO: 1202), GAGguauuua (SEQ ID NO: 1972), GAGgucuaca (SEQ ID NO: 3712), GUUguagguc (SEQ ID NO: 3713), CAGguacucg (SEQ ID NO: 3714), GUCguauguu (SEQ ID NO: 3715), AAGguacuuu (SEQ ID NO: 202), AGAgugagau (SEQ ID NO: 702), AGUguuggua (SEQ ID NO: 3716), AAUgugagug (SEQ ID NO: 525), AAGguagauu (SEQ ID NO: 3717), AUGguuugua (SEQ ID NO: 988), GAGgccccag (SEQ ID NO: 3718), AUGgucaguu (SEQ ID NO: 3719), UCUguaagga (SEQ ID NO: 3720), CAGgucgggc (SEQ ID NO: 3721), CAGguaagcc (SEQ ID NO: 1142), UAGgucagug (SEQ ID NO: 2569), AGAguaggaa (SEQ ID NO: 683), CUGguacuuc (SEQ ID NO: 3722), CUCguaagca (SEQ ID NO: 1674), CAGguaacua (SEQ ID NO: 1134), CAGguggcug (SEQ ID NO: 1401), UGGguccgua (SEQ ID NO: 3723), GAGguugugc (SEQ ID NO: 3724), CAGgugcgcg (SEQ ID NO: 1377), AAAguauggc (SEQ ID NO: 3725), UGAguacgua (SEQ ID NO: 2779), CUGguacgga (SEQ ID NO: 3726), CAAgugaccu (SEQ ID NO: 3727), AAGgugaugu (SEQ ID NO: 356), AAGgucugca (SEQ ID NO: 3728), AAAguuugua (SEQ ID NO: 75), AAGgugagca (SEQ ID NO: 339), GAUguaagcc (SEQ ID NO: 2119), CAAguaauuu (SEQ ID NO: 1035), CAGgugugug (SEQ ID NO: 1442), UGGgugaggg (SEQ ID NO: 2874), AAGgugaccu (SEQ ID NO: 3729), UAGgugugag (SEQ ID NO: 2621), CAGgcagguc (SEQ ID NO: 3730), UCAguaaguu (SEQ ID NO: 2692), UCAgcaguga (SEQ ID NO: 3731), AAGguaccac (SEQ ID NO: 3732), UAAguaggug (SEQ ID NO: 3733), AAGgucagcc (SEQ ID NO: 286), CAGguaacuc (SEQ ID NO: 1135), AAAguaagag (SEQ ID NO: 13), AAGguagaua (SEQ ID NO: 209), AAGgcaaggg (SEQ ID NO: 99), CAGgugucgg (SEQ ID NO: 3734), CAGguggcua (SEQ ID NO: 3735), GAGguugcca (SEQ ID NO: 3736), CAGgccgugg (SEQ ID NO: 3737), UUGguauaug (SEQ ID NO: 3738), GAGguugagu (SEQ ID NO: 3739), GAGguagguc (SEQ ID NO: 3740), GUGguaagac (SEQ ID NO: 2343), UAGguccuuc (SEQ ID NO: 3741), GAGgcaaguc (SEQ ID NO: 3742), GAGguaacau (SEQ ID NO: 3743), CAGguauauc (SEQ ID NO: 1236), UCGguugguu (SEQ ID NO: 3744), CAGgugaacc (SEQ ID NO: 3745), CAGgucuuuu (SEQ ID NO: 3746), CAGgcauggc (SEQ ID NO: 3747), AAAguacuug (SEQ ID NO: 32), CAGgugauuc (SEQ ID NO: 1356), UUGguagguu (SEQ ID NO: 3748), UAUgugagca (SEQ ID NO: 3749), CAGgugagcg (SEQ ID NO: 1339), AAUguaauaa (SEQ ID NO: 3750), AAAguaaggc (SEQ ID NO: 3751), UAGguuuguc (SEQ ID NO: 2644), UAGgugggag (SEQ ID NO: 2613), GAGguaaguu (SEQ ID NO: 3752), AAGguagccg (SEQ ID NO: 3753), CAGguggugc (SEQ ID NO: 3754), UGAgucaguu (SEQ ID NO: 3755), CUGguaggcc (SEQ ID NO: 3756), CAAguaagga (SEQ ID NO: 3757), CGGguaaggc (SEQ ID NO: 3758), AAGgcgagga (SEQ ID NO: 3759), CAGguaguuc (SEQ ID NO: 1230), CAGguaagga (SEQ ID NO: 1143), CCUgugagug (SEQ ID NO: 1610), AAGguaaaug (SEQ ID NO: 132), CCGguaauua (SEQ ID NO: 3760), CAGguaaguu (SEQ ID NO: 1149), AAGgugguca (SEQ ID NO: 3761), CAGguaccuc (SEQ ID NO: 1177), AUCguaagua (SEQ ID NO: 3762), CCGguacaua (SEQ ID NO: 3763), GCGgugagug (SEQ ID NO: 3764), GAGgugguau (SEQ ID NO: 2067), CUGgugugga (SEQ ID NO: 3765), GAGguaauuc (SEQ ID NO: 3766), CAAguacgua (SEQ ID NO: 3767), UCUguaagug (SEQ ID NO: 2746), AAUguaagug (SEQ ID NO: 491), AGGgucuguu (SEQ ID NO: 783), GAGguacugc (SEQ ID NO: 1918), AGGguaaggc (SEQ ID NO: 738), AAGgcaagag (SEQ ID NO: 95), CAGguggguu (SEQ ID NO: 1416), UAGguuagga (SEQ ID NO: 3768), UGAguaagcu (SEQ ID NO: 2769), AGAguaagag (SEQ ID NO: 661), AUGgcaggug (SEQ ID NO: 3769), UAGgcaagua (SEQ ID NO: 3770), AUGguaggua (SEQ ID NO: 923), GCAgcccgca (SEQ ID NO: 3771), ACGguaaacu (SEQ ID NO: 3772), AGGgugaguu (SEQ ID NO: 798), GUAguagucu (SEQ ID NO: 3773), GUGgcugaaa (SEQ ID NO: 3774), CAGguuaguc (SEQ ID NO: 1456), CUGgugagca (SEQ ID NO: 1753), UCAguaagug (SEQ ID NO: 2691), AAAgugauug (SEQ ID NO: 3775), UAGgucugga (SEQ ID NO: 3776), GAGguguuuc (SEQ ID NO: 3777), AAGguaaauu (SEQ ID NO: 133), CAUguacauc (SEQ ID NO: 3778), AAGguuugaa (SEQ ID NO: 3779), CCAgcaagug (SEQ ID NO: 3780), UAGguaauaa (SEQ ID NO: 3781), GAGgcaagug (SEQ ID NO: 1859), CAAgugauuc (SEQ ID NO: 1071), CAGgucgugg (SEQ ID NO: 3782), GAAguaugcc (SEQ ID NO: 3783), UCGgugcccu (SEQ ID NO: 3784), GAGgucaguc (SEQ ID NO: 3785), CAGgugagac (SEQ ID NO: 1334), UUUgucugua (SEQ ID NO: 3786), CAGguagaua (SEQ ID NO: 3787), UGGguaucag (SEQ ID NO: 3788), UAGgugggcu (SEQ ID NO: 2616), AUGgugagau (SEQ ID NO: 3789), CAGguaacac (SEQ ID NO: 3790), CCGguauccu (SEQ ID NO: 3791), UAGguaagcu (SEQ ID NO: 2487), UCAguacauc (SEQ ID NO: 3792), UAGguuugcc (SEQ ID NO: 2642), AUGguaagaa (SEQ ID NO: 889), UUGguaagac (SEQ ID NO: 3793), CCGguuaguc (SEQ ID NO: 3794), GAGguaagaa (SEQ ID NO: 1882), UGGguaaguu (SEQ ID NO: 2844), CCGgugagaa (SEQ ID NO: 1585), CCUgugaggg (SEQ ID NO: 1608), ACGguaggag (SEQ ID NO: 590), ACAguauguc (SEQ ID NO: 3795), CAGguauuaa (SEQ ID NO: 3796), CAGguggauc (SEQ ID NO: 3797), AGAgugcgua (SEQ ID NO: 3798), AAGgugaccg (SEQ ID NO: 3799), AGAguaggug (SEQ ID NO: 687), ACUguaugua (SEQ ID NO: 3800), UAGgucaauu (SEQ ID NO: 3801), AGUguguaag (SEQ ID NO: 3802), CGGguaccuu (SEQ ID NO: 3803), CUAgugaguu (SEQ ID NO: 3804), CUAguaagug (SEQ ID NO: 1666), CAGguacaac (SEQ ID NO: 3805), UAGgugugug (SEQ ID NO: 2627), CAUguacggc (SEQ ID NO: 3806), AUGgugugag (SEQ ID NO: 3807), AGGguggaag (SEQ ID NO: 3808), CAGgugcgag (SEQ ID NO: 3809), UAGgugcucc (SEQ ID NO: 3810), AAGguggugg (SEQ ID NO: 390), AAGgucuguu (SEQ ID NO: 317), CAGgugggcc (SEQ ID NO: 1407), AAGgucaguc (SEQ ID NO: 294), CAGguuuuua (SEQ ID NO: 3811), AACgugaggu (SEQ ID NO: 3812), CGGguaagag (SEQ ID NO: 3813), UUUgucggua (SEQ ID NO: 3814), UAGguuaagu (SEQ ID NO: 3815), GUGguaagaa (SEQ ID NO: 2342), CAGguauugg (SEQ ID NO: 1266), GCUguaaguu (SEQ ID NO: 2196), CUAguaagua (SEQ ID NO: 1664), UCGguaaaua (SEQ ID NO: 3816), CAGguaacuu (SEQ ID NO: 1137), CCUgugagua (SEQ ID NO: 3817), CAGguuauau (SEQ ID NO: 3818), CUGgugaaca (SEQ ID NO: 3819), AAGguauaaa (SEQ ID NO: 238), GAGguaagca (SEQ ID NO: 1885), AAGgugaagc (SEQ ID NO: 324), CAGgugaguu (SEQ ID NO: 1348), UUUgugagua (SEQ ID NO: 3820), CUUguacgcc (SEQ ID NO: 3821), AGAguaagug (SEQ ID NO: 670), UGGguaggug (SEQ ID NO: 2853), UGAgcccuge (SEQ ID NO: 3822), UGUguaugua (SEQ ID NO: 3823), AAGguagagg (SEQ ID NO: 3824), GAGguggggg (SEQ ID NO: 2062), UAGguaauuc (SEQ ID NO: 2502), AAGgcauggu (SEQ ID NO: 3825), AGAguaagca (SEQ ID NO: 663), AAGguaggaa (SEQ ID NO: 217), CAAguaagua (SEQ ID NO: 1026), ACUguaauug (SEQ ID NO: 3826), CAGgucugug (SEQ ID NO: 1311), UCGguaccga (SEQ ID NO: 3827), CUGgugagag (SEQ ID NO: 3828), AAGguuugcu (SEQ ID NO: 463), AUGguaccac (SEQ ID NO: 3829), UAAguuaguu (SEQ ID NO: 3830), CAGguaggac (SEQ ID NO: 1213), AGAgugaggc (SEQ ID NO: 3831), CGAgucagua (SEQ ID NO: 3832), CAGgucugag (SEQ ID NO: 1304), GAGguggugg (SEQ ID NO: 3833), ACGguauugg (SEQ ID NO: 3834), GCUgcgagua (SEQ ID NO: 3835), CUGguaagug (SEQ ID NO: 1708), GUGgugagau (SEQ ID NO: 2379), GGGguuugau (SEQ ID NO: 3836), UCUgugagug (SEQ ID NO: 2762), CUUgucagua (SEQ ID NO: 1801), GAGguaaaac (SEQ ID NO: 1866), UCUguaagau (SEQ ID NO: 2741), CCAguaaguu (SEQ ID NO: 1558), CAGguaaagu (SEQ ID NO: 1124), GCGgugagca (SEQ ID NO: 2179), UAAguaagag (SEQ ID NO: 2416), CUGgcaggug (SEQ ID NO: 3837), GAGguaaggg (SEQ ID NO: 1891), UGAguaaguu (SEQ ID NO: 2775), GAGgugagac (SEQ ID NO: 2015), GCUgucuguu (SEQ ID NO: 3838), AAGguaacaa (SEQ ID NO: 134), GAGguaacgg (SEQ ID NO: 3839), CUGguauucu (SEQ ID NO: 3840), CAAguaacug (SEQ ID NO: 1021), AAGguggggu (SEQ ID NO: 383), UAGguauggc (SEQ ID NO: 2549), CAGguauuuu (SEQ ID NO: 1271), GUGguaaacu (SEQ ID NO: 3841), GAGgucugag (SEQ ID NO: 1998), CUGguaaggu (SEQ ID NO: 1706), CAAguaaguu (SEQ ID NO: 1029), AAGguagacc (SEQ ID NO: 206), GAGgcgagcg (SEQ ID NO: 3842), CUGguaaaua (SEQ ID NO: 1687), UGUguaagcg (SEQ ID NO: 3843), CAGguuaggg (SEQ ID NO: 1453), GGGgugagga (SEQ ID NO: 2280), ACAguaugug (SEQ ID NO: 3844), CCGgugggga (SEQ ID NO: 3845), GAGgucagug (SEQ ID NO: 3846), AGGguaaggu (SEQ ID NO: 3847), ACAguaagua (SEQ ID NO: 546), GGUguaaggu (SEQ ID NO: 3848), GAGguaauaa (SEQ ID NO: 1895), CAGguauucc (SEQ ID NO: 3849), CUGguauaaa (SEQ ID NO: 3850), CCGgucugug (SEQ ID NO: 3851), CAGguaacug (SEQ ID NO: 1136), GCAguaagua (SEQ ID NO: 2147), AAGguagggg (SEQ ID NO: 225), CAAguccacc (SEQ ID NO: 3852), CAAguuggug (SEQ ID NO: 3853), CAGgugcggu (SEQ ID NO: 1379), CAGguaaaau (SEQ ID NO: 3854), ACGguaagga (SEQ ID NO: 3855), UGGguaauaa (SEQ ID NO: 3856), UAGguaagug (SEQ ID NO: 2493), CCGguagguu (SEQ ID NO: 3857), AGAguaugga (SEQ ID NO: 3858), CUCgugaguc (SEQ ID NO: 3859), AAAgccggug (SEQ ID NO: 3860), UUGguaauuu (SEQ ID NO: 2970), GAGguaaaag (SEQ ID NO: 1867), CCUgugugag (SEQ ID NO: 3861), AAAguaagga (SEQ ID NO: 18), UGAgugagug (SEQ ID NO: 2800), AAGguacaug (SEQ ID NO: 180), CCGguaaaug (SEQ ID NO: 3862), CAGgugaagc (SEQ ID NO: 3863), CAGguacccg (SEQ ID NO: 1173), GAGguaaggc (SEQ ID NO: 1890), UUUguauguu (SEQ ID NO: 3049), CAGgugcucc (SEQ ID NO: 1386), UCGguagguc (SEQ ID NO: 3864), CGGgugaggc (SEQ ID NO: 3865), AAGguaauua (SEQ ID NO: 168), ACUgugaguc (SEQ ID NO: 644), AAGgucagca (SEQ ID NO: 285), GUGgugagug (SEQ ID NO: 2384), CAUguccacc (SEQ ID NO: 3866), AAGgugaccc (SEQ ID NO: 3867), CGGguuagua (SEQ ID NO: 3868), GCGguaguaa (SEQ ID NO: 3869), GCUguaggua (SEQ ID NO: 3870), CCUguugagu (SEQ ID NO: 3871), UAGgucuggc (SEQ ID NO: 2577), GAUgugagcc (SEQ ID NO: 2131), CUUgugagua (SEQ ID NO: 1802), CUGguguguu (SEQ ID NO: 1780), GAGgcaugug (SEQ ID NO: 1863), CAGgcaagag (SEQ ID NO: 1101), UUGguaagaa (SEQ ID NO: 2957), GAGguguggg (SEQ ID NO: 2075), GAGguauuuu (SEQ ID NO: 1975), CAGguaguaa (SEQ ID NO: 1224), AGGguaagac (SEQ ID NO: 3872), UUUguaggca (SEQ ID NO: 3873), AGGgugagau (SEQ ID NO: 3874), GAGguuugua (SEQ ID NO: 2110), AAGgugagug (SEQ ID NO: 349), GAGgugggag (SEQ ID NO: 2055), AAGgugagaa (SEQ ID NO: 335), CUGguaagag (SEQ ID NO: 1698), AUAguaaaga (SEQ ID NO: 3875), GAUgugaguc (SEQ ID NO: 2134), AAGgugcagg (SEQ ID NO: 3876), CAGgucuguc (SEQ ID NO: 1310), GAGgugauuu (SEQ ID NO: 3877), CAGguuggcu (SEQ ID NO: 3878), CGGguauggg (SEQ ID NO: 3879), AUGguccauc (SEQ ID NO: 3880), CCGguuggug (SEQ ID NO: 3881), GGAguaaguc (SEQ ID NO: 3882), AAUguaagga (SEQ ID NO: 488), CAGguuuguu (SEQ ID NO: 1510), UAGgugugua (SEQ ID NO: 2626), UAUgucuuug (SEQ ID NO: 3883), ACGguacuuc (SEQ ID NO: 3884), AAGgcacgcg (SEQ ID NO: 3885), CUGguaaacc (SEQ ID NO: 1684), CUUgugggua (SEQ ID NO: 3886), UGAguaaguc (SEQ ID NO: 2773), CUGgugggug (SEQ ID NO: 1773), GAGguggaga (SEQ ID NO: 3887), GUGguggcug (SEQ ID NO: 3888), GUGguaagug (SEQ ID NO: 2353), AACgugagua (SEQ ID NO: 3889), GAAgcuguaa (SEQ ID NO: 3890), CGGguaucuu (SEQ ID NO: 3891), CAGgugucag (SEQ ID NO: 1424), AAUguacgca (SEQ ID NO: 3892), CCGgugggua (SEQ ID NO: 3893), UGGgugaggu (SEQ ID NO: 3894), AAGguauguu (SEQ ID NO: 266), CAGguauguu (SEQ ID NO: 1261), CAGguuugcu (SEQ ID NO: 1505), UUGguaaguu (SEQ ID NO: 2964), CAGguaguug (SEQ ID NO: 1231), CCUgugaaua (SEQ ID NO: 3895), GCUgugugug (SEQ ID NO: 3896), CAAguaauuc (SEQ ID NO: 1033), AGGguaaugu (SEQ ID NO: 3897), GCUgugaguc (SEQ ID NO: 2205), ACCguaaguu (SEQ ID NO: 3898), CGUguaagua (SEQ ID NO: 3899), GGGguaaguc (SEQ ID NO: 3900), AAUguaugau (SEQ ID NO: 3901), AAUgugauua (SEQ ID NO: 3902), UCAguaagaa (SEQ ID NO: 2682), CAGguccguc (SEQ ID NO: 3903), GAAguauuga (SEQ ID NO: 3904), UUGguaagga (SEQ ID NO: 2960), CAGgucgguu (SEQ ID NO: 3905), UAGguuagug (SEQ ID NO: 2635), ACGguaaaac (SEQ ID NO: 577), AAGguagguc (SEQ ID NO: 228), UACgugagua (SEQ ID NO: 3906), UUGguaagca (SEQ ID NO: 3907), GCGgugaguc (SEQ ID NO: 3908), GAAguaaggg (SEQ ID NO: 3909), CGCgugaguu (SEQ ID NO: 3910), CAGguacccc (SEQ ID NO: 3911), UCUguaagac (SEQ ID NO: 3912), GAGgugggca (SEQ ID NO: 2057), AAUguaagac (SEQ ID NO: 3913), CAGgcaaggg (SEQ ID NO: 3914), CAAguaacua (SEQ ID NO: 1020), AAAguuuguc (SEQ ID NO: 3915), CAGguacugu (SEQ ID NO: 1193), AAGgucccuc (SEQ ID NO: 303), UCGguaaguc (SEQ ID NO: 3916), UGGgugagug (SEQ ID NO: 2877), CUUgugagau (SEQ ID NO: 3917), AGAgugagcu (SEQ ID NO: 3918), UAAgugggga (SEQ ID NO: 3919), UAGguaggga (SEQ ID NO: 2522), CAGguuagcc (SEQ ID NO: 1452), AGGguaauca (SEQ ID NO: 3920), AAGguucagc (SEQ ID NO: 3921), UGGgugggug (SEQ ID NO: 2885), CAGguuguga (SEQ ID NO: 1494), AAGguaagug (SEQ ID NO: 155), CAUgugcgua (SEQ ID NO: 1543), CCGguauauu (SEQ ID NO: 3922), ACCguaugug (SEQ ID NO: 3923), CAGguauagu (SEQ ID NO: 3924), CAGguauuac (SEQ ID NO: 3925), CAGgugcagg (SEQ ID NO: 1364), GUGgugagcu (SEQ ID NO: 2381), AAGguaacau (SEQ ID NO: 135), CUGgugaugg (SEQ ID NO: 3926), AUGguaaaug (SEQ ID NO: 882), CCGgugagca (SEQ ID NO: 3927), AAGguaaacc (SEQ ID NO: 124), AAGguacugg (SEQ ID NO: 3928), GCGgucagga (SEQ ID NO: 3929), CUGgucaggg (SEQ ID NO: 3930), AAAguacguu (SEQ ID NO: 3931), AGAguagguu (SEQ ID NO: 688), AGGguaagcu (SEQ ID NO: 3932), AUUgugagua (SEQ ID NO: 1009), CCGgccacca (SEQ ID NO: 3933), GAGguaacuu (SEQ ID NO: 1881), GAGguaugaa (SEQ ID NO: 1956), CAGgucagac (SEQ ID NO: 1276), UAGgcgugug (SEQ ID NO: 2462), AGGguaaguu (SEQ ID NO: 743), CAGgcaugag (SEQ ID NO: 1111), CAGguaacgu (SEQ ID NO: 1133), CAGgcgagca (SEQ ID NO: 3934), UAGguauggu (SEQ ID NO: 2550), AGAguaggau (SEQ ID NO: 3935), CUGguuucaa (SEQ ID NO: 3936), GAGguaaacu (SEQ ID NO: 3937), CAGgcaugca (SEQ ID NO: 1112), UUGguaaucu (SEQ ID NO: 3938), AGGgcagaau (SEQ ID NO: 3939), AUGguaaaac (SEQ ID NO: 877), GCUgcaggug (SEQ ID NO: 3940), GAAgcacgug (SEQ ID NO: 3941), CAUguaaaca (SEQ ID NO: 3942), UGGguaagau (SEQ ID NO: 2835), AGGguagcua (SEQ ID NO: 3943), AGGguggggu (SEQ ID NO: 800), CCUguaaguu (SEQ ID NO: 1600), UGAgugaguu (SEQ ID NO: 2801), GGAguaugua (SEQ ID NO: 3944), CAGgugaccu (SEQ ID NO: 1328), AAAguacgga (SEQ ID NO: 3945), GAGguacaga (SEQ ID NO: 1906), GAUguaggua (SEQ ID NO: 2125), GGGguaauug (SEQ ID NO: 3946), UAGguggguu (SEQ ID NO: 2617), GUGguacgua (SEQ ID NO: 3947), AAGguacagc (SEQ ID NO: 3948), GAGgugaaga (SEQ ID NO: 3949), GGGguaagca (SEQ ID NO: 2246), UGAguagguc (SEQ ID NO: 3950), GGGguaaguu (SEQ ID NO: 2253), AUUgugaguu (SEQ ID NO: 1011), UCAguaagac (SEQ ID NO: 3951), AGUgugagcu (SEQ ID NO: 834), AAGgcaaaac (SEQ ID NO: 3952), CUGgugaguc (SEQ ID NO: 1760), AAGgucucug (SEQ ID NO: 310), GAGgcugugc (SEQ ID NO: 3953), AGAgugagac (SEQ ID NO: 700), GAGgugaugu (SEQ ID NO: 2033), AGAguauggu (SEQ ID NO: 3954), UGGguggguc (SEQ ID NO: 2884), GCUgcugagc (SEQ ID NO: 3955), CAGguagcug (SEQ ID NO: 1210), UAGgucagaa (SEQ ID NO: 3956), CCGguaggug (SEQ ID NO: 3957), GCAguaugau (SEQ ID NO: 3958), CAGguuucag (SEQ ID NO: 3959), GAGguuugcc (SEQ ID NO: 3960), GGGguggggg (SEQ ID NO: 3961), AAGguacaua (SEQ ID NO: 179), UGGguguguu (SEQ ID NO: 2890), AGAguaaggc (SEQ ID NO: 666), GCGguuagug (SEQ ID NO: 3962), AAGgugacuu (SEQ ID NO: 334), AUGguaagau (SEQ ID NO: 892), AUGguaguug (SEQ ID NO: 3963), CAUguaagac (SEQ ID NO: 3964), CUGguaugua (SEQ ID NO: 1736), UUCguaagga (SEQ ID NO: 3965), GAAguaugac (SEQ ID NO: 3966), CGGguaauuc (SEQ ID NO: 1627), UGGguaacuu (SEQ ID NO: 2831), CAGgugccua (SEQ ID NO: 1372), CAUguagggc (SEQ ID NO: 3967), ACCgucagga (SEQ ID NO: 3968), CGUguucgau (SEQ ID NO: 3969), GAGgcaggac (SEQ ID NO: 3970), UAGguaauau (SEQ ID NO: 2496), UCGguauacu (SEQ ID NO: 3971), UAGguugugc (SEQ ID NO: 3972), CCGgugaguc (SEQ ID NO: 3973), CAGgugccaa (SEQ ID NO: 1368), CAGgugaugc (SEQ ID NO: 1352), AAGgugagga (SEQ ID NO: 343), GUGgugaggg (SEQ ID NO: 3974), UGGgucagua (SEQ ID NO: 3975), GAGgucaggg (SEQ ID NO: 1985), UAGguacgua (SEQ ID NO: 2511), GAGgcaagag (SEQ ID NO: 1857), CCUguuggua (SEQ ID NO: 3976), GAGguaucca (SEQ ID NO: 3977), UAAguaagcu (SEQ ID NO: 2419), AAGgucaguu (SEQ ID NO: 296), AAAguuaaag (SEQ ID NO: 3978), GAGgugcuau (SEQ ID NO: 3979), ACGguaaguu (SEQ ID NO: 581), CUGgugaggg (SEQ ID NO: 1757), GAGguuaugu (SEQ ID NO: 2091), CUUgugugca (SEQ ID NO: 3980), UGAgcugggg (SEQ ID NO: 3981), AAGguauagu (SEQ ID NO: 3982), UAGguaaaac (SEQ ID NO: 2464), GGGgugaggu (SEQ ID NO: 3983), GAGgcaagca (SEQ ID NO: 3984), GGAguaacgu (SEQ ID NO: 3985), AGAguaagua (SEQ ID NO: 3986), AAAguaagua (SEQ ID NO: 21), GAGgcaacca (SEQ ID NO: 3987), UGUguaaguu (SEQ ID NO: 2909), UAGgugaggc (SEQ ID NO: 2594), ACAguaagaa (SEQ ID NO: 544), UGAguaagug (SEQ ID NO: 2774), CAAgucagua (SEQ ID NO: 1057), AGGguaaaug (SEQ ID NO: 3988), AAGguaugca (SEQ ID NO: 257), GCUgugcgug (SEQ ID NO: 3989), GAGguucgcc (SEQ ID NO: 3990), AAGgcuugca (SEQ ID NO: 3991), CAGgcaagug (SEQ ID NO: 1104), AUAguaaguc (SEQ ID NO: 3992), UUGguaggua (SEQ ID NO: 2978), GCAgcaggua (SEQ ID NO: 3993), AAGguauauc (SEQ ID NO: 243), AGCguaagcc (SEQ ID NO: 3994), CUGguucgaa (SEQ ID NO: 3995), ACGgugggug (SEQ ID NO: 612), CUGgucauug (SEQ ID NO: 3996), CAGgucagga (SEQ ID NO: 1280), CAAgugagac (SEQ ID NO: 1062), GAGguacugg (SEQ ID NO: 1919), GAGguguagu (SEQ ID NO: 3997), GAGguguccu (SEQ ID NO: 3998), CAGgugcgua (SEQ ID NO: 1380), AGUgcccuga (SEQ ID NO: 3999), AUGgugaguc (SEQ ID NO: 962), UGUgugugua (SEQ ID NO: 4000), CAGguaugcu (SEQ ID NO: 1254), CUGguacagu (SEQ ID NO: 4001), UUGguacgua (SEQ ID NO: 4002), UCUguacgua (SEQ ID NO: 4003), UAAguaauuc (SEQ ID NO: 4004), CACguaugug (SEQ ID NO: 4005), CAGgcaagua (SEQ ID NO: 1103), UCGgugagug (SEQ ID NO: 4006), GGUgugaguc (SEQ ID NO: 2315), UCUguaagcu (SEQ ID NO: 2743), AAGguucaga (SEQ ID NO: 4007), AGGguacuuc (SEQ ID NO: 4008), GCGgcagguu (SEQ ID NO: 4009), GAGgcccgug (SEQ ID NO: 4010), CAGguauaaa (SEQ ID NO: 4011), AUGgucaagu (SEQ ID NO: 4012), AAGgugagua (SEQ ID NO: 347), GUGguuuguu (SEQ ID NO: 4013), AGAgugagga (SEQ ID NO: 4014), GAGguaugac (SEQ ID NO: 1957), UAGgcgugag (SEQ ID NO: 4015), AAGguacucc (SEQ ID NO: 4016), UGAgugagga (SEQ ID NO: 2798), GAGguaugau (SEQ ID NO: 4017), GGGgucggua (SEQ ID NO: 4018), ACGguaugca (SEQ ID NO: 4019), CAGguaccac (SEQ ID NO: 1171), UAAguaccug (SEQ ID NO: 4020), AGGgugggcu (SEQ ID NO: 4021), CUGgucuguu (SEQ ID NO: 4022), UAGgucagag (SEQ ID NO: 4023), AAGguguguu (SEQ ID NO: 406), CUGgucagug (SEQ ID NO: 4024), AAGgugggac (SEQ ID NO: 4025), GUGguaguag (SEQ ID NO: 4026), CUAguuuagg (SEQ ID NO: 4027), CCCgccccau (SEQ ID NO: 4028), GCUguacugc (SEQ ID NO: 4029), GAGguaauau (SEQ ID NO: 1897), UAGguuggug (SEQ ID NO: 4030), AAGguccaac (SEQ ID NO: 4031), UAGgugagga (SEQ ID NO: 2593), GUGguaaguu (SEQ ID NO: 2354), AGUgugagag (SEQ ID NO: 831), AAUguacaug (SEQ ID NO: 497), UUGgcaggug (SEQ ID NO: 4032), UAGguuauug (SEQ ID NO: 4033), CAGguacuga (SEQ ID NO: 1191), GCGguggguc (SEQ ID NO: 4034), UGUguaagau (SEQ ID NO: 4035), GAGgugagua (SEQ ID NO: 2025), GCAgccccgg (SEQ ID NO: 4036), CAGgugcuaa (SEQ ID NO: 4037), AGUguaagag (SEQ ID NO: 815), CAGguacauc (SEQ ID NO: 4038), CAGgugggac (SEQ ID NO: 1403), AGGguaaaua (SEQ ID NO: 727), UAAguaauua (SEQ ID NO: 4039), CAGguaaccg (SEQ ID NO: 1132), AAGguuugca (SEQ ID NO: 461), UAGgugguuu (SEQ ID NO: 4040), CAGgugaccg (SEQ ID NO: 1327), UGUguaagcu (SEQ ID NO: 4041), GGAgugaguc (SEQ ID NO: 2227), AGGguaggag (SEQ ID NO: 752), AGGgugggug (SEQ ID NO: 802), AAGgucugag (SEQ ID NO: 313), GAUguaauau (SEQ ID NO: 4042), GGGguaauua (SEQ ID NO: 4043), UAGguaggua (SEQ ID NO: 2524), GAGgcaagua (SEQ ID NO: 1858), GAGguaagga (SEQ ID NO: 1889), UAGguacuac (SEQ ID NO: 4044), UCGgugggug (SEQ ID NO: 4045), AAGgugugga (SEQ ID NO: 401), CAGgucugcc (SEQ ID NO: 1305), UAAgugagcc (SEQ ID NO: 4046), GAAguaaguu (SEQ ID NO: 1820), GAAguaagcc (SEQ ID NO: 1815), UAGgugcgac (SEQ ID NO: 4047), GAGguauggc (SEQ ID NO: 4048), GCAguaagaa (SEQ ID NO: 2145), CAGgugugga (SEQ ID NO: 1438), UUGguaacgu (SEQ ID NO: 4049), GCUguaaaaa (SEQ ID NO: 4050), UUGguuagua (SEQ ID NO: 4051), AUAguaaggg (SEQ ID NO: 4052), UUGguacuag (SEQ ID NO: 4053), CGGgcagccg (SEQ ID NO: 4054), CAGgugcugg (SEQ ID NO: 1389), UAUgugaguu (SEQ ID NO: 2673), CAGgucuggg (SEQ ID NO: 4055), UAAguaagaa (SEQ ID NO: 2415), AAGguuauua (SEQ ID NO: 4056), AGAguaaagc (SEQ ID NO: 4057), AGAgugugag (SEQ ID NO: 4058), UAGgugcgag (SEQ ID NO: 4059), CAAguaaacg (SEQ ID NO: 4060), AAGguacgua (SEQ ID NO: 4061), CUGgugagua (SEQ ID NO: 1759), CCAguaugua (SEQ ID NO: 4062), UUGgugagug (SEQ ID NO: 3006), UGAguaagua (SEQ ID NO: 2772), GAGguuagca (SEQ ID NO: 4063), GUGguaagcc (SEQ ID NO: 4064), CUGguauggc (SEQ ID NO: 1734), AAAguaacac (SEQ ID NO: 8), CAGguacuaa (SEQ ID NO: 1186), UCUguaaguu (SEQ ID NO: 2747), GAGgugaggg (SEQ ID NO: 2024), ACUgugggua (SEQ ID NO: 647), GAUguuugug (SEQ ID NO: 4065), CAGgugucaa (SEQ ID NO: 4066), CAGgucacca (SEQ ID NO: 4067), CCGgugagua (SEQ ID NO: 4068), UUGguaaaua (SEQ ID NO: 4069), CAGguggggg (SEQ ID NO: 1411), ACUgcaggug (SEQ ID NO: 4070), UAGguauguu (SEQ ID NO: 2554), GGAgcaagug (SEQ ID NO: 4071), UCGgugccuc (SEQ ID NO: 4072), CAAguaacuu (SEQ ID NO: 4073), GAGguaacca (SEQ ID NO: 1879), CAGguaauau (SEQ ID NO: 1151), GGAguaagaa (SEQ ID NO: 4074), GAGguaccuu (SEQ ID NO: 1914), AGGguaagga (SEQ ID NO: 737), CCUgugaguc (SEQ ID NO: 1609), GAGguaaugg (SEQ ID NO: 1900), AUGguguguc (SEQ ID NO: 4075), GGGgugagua (SEQ ID NO: 4076), AGGgucaggu (SEQ ID NO: 4077), UGGguaaggg (SEQ ID NO: 2839), AGGguagguu (SEQ ID NO: 759), AUAgugaguu (SEQ ID NO: 4078), CCCguaggcu (SEQ ID NO: 4079), ACAguaugua (SEQ ID NO: 553), GACgugugua (SEQ ID NO: 4080), GCGgugagga (SEQ ID NO: 4081), CAGgugaccc (SEQ ID NO: 1326), UAAguuuagu (SEQ ID NO: 4082), ACAguugagu (SEQ ID NO: 570), CGGgugaggg (SEQ ID NO: 1639), CAGguggauu (SEQ ID NO: 1398), CGGguagagg (SEQ ID NO: 4083), UAGgugcgug (SEQ ID NO: 2608), GGGguaagaa (SEQ ID NO: 2243), GAGguggggu (SEQ ID NO: 4084), CACguggguu (SEQ ID NO: 4085), ACGguaauug (SEQ ID NO: 4086), AGAgugaguc (SEQ ID NO: 705), UUGgcuccaa (SEQ ID NO: 4087), AAGgugaugc (SEQ ID NO: 355), AAGguugguc (SEQ ID NO: 448), AGCguaaguu (SEQ ID NO: 4088), AUUguaugua (SEQ ID NO: 1006), UCAguuaagu (SEQ ID NO: 4089), CAAguacgug (SEQ ID NO: 4090), CAGgugcgug (SEQ ID NO: 1382), CAGguaggua (SEQ ID NO: 1220), AUGguggggu (SEQ ID NO: 4091), AUGgugaguu (SEQ ID NO: 964), CAGguaauca (SEQ ID NO: 4092), AAGguagggu (SEQ ID NO: 226), CAGgccaagg (SEQ ID NO: 4093), GUGgugagag (SEQ ID NO: 4094), AAGguuggug (SEQ ID NO: 449), CAGguacucu (SEQ ID NO: 1190), UAGgcaugug (SEQ ID NO: 4095), UUGguaccuu (SEQ ID NO: 4096), CUGgugugcc (SEQ ID NO: 4097), ACAguugcca (SEQ ID NO: 4098), UUGguaauau (SEQ ID NO: 4099), GAGgugcaug (SEQ ID NO: 4100), UUGguuugua (SEQ ID NO: 3028), UUGguaagug (SEQ ID NO: 2963), UGUgugugug (SEQ ID NO: 4101), GUGguuugua (SEQ ID NO: 2398), GCGguacaca (SEQ ID NO: 4102), AGAguaugcu (SEQ ID NO: 4103), UUUguaagua (SEQ ID NO: 3038), UCUgugcggg (SEQ ID NO: 4104), AAGgucagug (SEQ ID NO: 295), GAGguaggaa (SEQ ID NO: 1930), GCGguuagca (SEQ ID NO: 4105), AGGgugaggg (SEQ ID NO: 793), GAAgugagua (SEQ ID NO: 4106), CAGgugacag (SEQ ID NO: 4107), AAGgugauua (SEQ ID NO: 357), GAGgccagcc (SEQ ID NO: 4108), GAGgucuccu (SEQ ID NO: 4109), UAGguauuac (SEQ ID NO: 2556), CAUguaagag (SEQ ID NO: 1519), CUGguagggc (SEQ ID NO: 4110), GAAguaagua (SEQ ID NO: 1818), CGGguaagug (SEQ ID NO: 4111), CAGguaaucu (SEQ ID NO: 4112), GUGguaggua (SEQ ID NO: 4113), CAGgugggua (SEQ ID NO: 1413), AAGgccagug (SEQ ID NO: 4114), AAAgugaauc (SEQ ID NO: 4115), ACGguuacgu (SEQ ID NO: 4116), AUGguaggaa (SEQ ID NO: 917), CGGgugagac (SEQ ID NO: 4117), GAGguuggaa (SEQ ID NO: 2099), UGGgugagcc (SEQ ID NO: 2871), CCAgugagua (SEQ ID NO: 1564), CUAguacgag (SEQ ID NO: 4118), CAGguaugac (SEQ ID NO: 1248), GCUgugaggu (SEQ ID NO: 4119), CUGguaugaa (SEQ ID NO: 4120), GGUguacgac (SEQ ID NO: 4121), CUUgugagug (SEQ ID NO: 4122), GUGgugagca (SEQ ID NO: 2380), CUGguaacuu (SEQ ID NO: 1696), CAGguacuau (SEQ ID NO: 1188), AGGguaaggg (SEQ ID NO: 739), UUGguuaguu (SEQ ID NO: 3025), GGUguaagca (SEQ ID NO: 2302), UCGgugagga (SEQ ID NO: 4123), UGGguaaaca (SEQ ID NO: 4124), UCGguacgug (SEQ ID NO: 4125), UAGguagcag (SEQ ID NO: 4126), CUGguaaggc (SEQ ID NO: 1704), GUGguaagga (SEQ ID NO: 2349), UAAguaagca (SEQ ID NO: 2418), GAGguuccaa (SEQ ID NO: 4127), CUGguaugga (SEQ ID NO: 4128), GGGgugggua (SEQ ID NO: 2288), CAGguuuccc (SEQ ID NO: 4129), CAGgucucug (SEQ ID NO: 4130), GAGgugagga (SEQ ID NO: 2022), CUUguggguu (SEQ ID NO: 1805), AUGgugagac (SEQ ID NO: 953), CAGgugaagg (SEQ ID NO: 1319), GCGguagggg (SEQ ID NO: 4131), GUUguuuccc (SEQ ID NO: 4132), AAAgcaucca (SEQ ID NO: 4133), GUGguagguu (SEQ ID NO: 2367), AAGgugugaa (SEQ ID NO: 398), CAGguacagu (SEQ ID NO: 1167), AAGguaccaa (SEQ ID NO: 182), UUGguaauug (SEQ ID NO: 2969), AAGgugcuca (SEQ ID NO: 4134), AAGguucaac (SEQ ID NO: 4135), CAGguuuaca (SEQ ID NO: 4136), GCUguaagug (SEQ ID NO: 2195), AGGguauguc (SEQ ID NO: 769), GAGgucgggg (SEQ ID NO: 1996), AAGgugccug (SEQ ID NO: 363), AAGguaaaaa (SEQ ID NO: 119), GUGgugaguu (SEQ ID NO: 2385), UAGguaagaa (SEQ ID NO: 4137), AGGguauccu (SEQ ID NO: 4138), GUGguaauau (SEQ ID NO: 4139), UCUguaagua (SEQ ID NO: 2744), UGGguaugga (SEQ ID NO: 4140), AUGguaugga (SEQ ID NO: 935), GACgugagcc (SEQ ID NO: 1854), CUGguuuggc (SEQ ID NO: 4141), AUGguauauc (SEQ ID NO: 4142), AAAguaaacu (SEQ ID NO: 4143), AGCgugagug (SEQ ID NO: 721), CUGguauaga (SEQ ID NO: 4144), CAGgugggga (SEQ ID NO: 1409), AGAguauguu (SEQ ID NO: 696), UAGguacuug (SEQ ID NO: 4145), GCAguaggug (SEQ ID NO: 4146), AGUguauguc (SEQ ID NO: 4147), AAGguuaagc (SEQ ID NO: 413), CUGguggccu (SEQ ID NO: 4148), GAAgugaguc (SEQ ID NO: 1839), UUGguguaag (SEQ ID NO: 4149), CAGguaagaa (SEQ ID NO: 1138), CGGgucucgg (SEQ ID NO: 4150), GAGgugcaca (SEQ ID NO: 2035), CUCguuaguu (SEQ ID NO: 4151), AAGgugauca (SEQ ID NO: 352), UAUguaagaa (SEQ ID NO: 2649), GAGgugcuug (SEQ ID NO: 2047), CAGgugguca (SEQ ID NO: 4152), ACGguaaguc (SEQ ID NO: 4153), ACAguaaugu (SEQ ID NO: 4154), CCUguaaggu (SEQ ID NO: 4155), GAGguuaagu (SEQ ID NO: 4156), UCGguaugug (SEQ ID NO: 2725), UGGguauguu (SEQ ID NO: 2863), AAGguauuac (SEQ ID NO: 268), CAGgugaggg (SEQ ID NO: 1343), UUGguaaaca (SEQ ID NO: 4157), AAGguagugu (SEQ ID NO: 4158), GAGguguggc (SEQ ID NO: 4159), CAGguacgga (SEQ ID NO: 4160), AAGgucauca (SEQ ID NO: 4161), CAAguaggca (SEQ ID NO: 4162), CAGgugaaac (SEQ ID NO: 4163), CAGguacugc (SEQ ID NO: 1192), AAUgcaagug (SEQ ID NO: 4164), CAUguaauuc (SEQ ID NO: 4165), AAGguaugcu (SEQ ID NO: 259), CUGgugaguu (SEQ ID NO: 1762), CAGgugguuu (SEQ ID NO: 4166), UGUgugagua (SEQ ID NO: 2922), AAGgucggug (SEQ ID NO: 4167), AUGguaaauu (SEQ ID NO: 883), AGGguauuac (SEQ ID NO: 771), AGUguaugga (SEQ ID NO: 4168), AACguaagau (SEQ ID NO: 4169), GUGguaaggu (SEQ ID NO: 4170), ACUguuagua (SEQ ID NO: 4171), CAGguaucag (SEQ ID NO: 1239), AAGguuaguu (SEQ ID NO: 425), CUGgugagcu (SEQ ID NO: 1754), UUGgugagcu (SEQ ID NO: 4172), UGUguacgua (SEQ ID NO: 4173), GAGgucagcc (SEQ ID NO: 4174), GAGguagaau (SEQ ID NO: 4175), AAGguaugag (SEQ ID NO: 255), UAGguauuuc (SEQ ID NO: 2563), UGUguaacac (SEQ ID NO: 4176), AGUguaaggc (SEQ ID NO: 4177), GAGgucugcu (SEQ ID NO: 4178), AAGguuagca (SEQ ID NO: 418), CAGguaaaug (SEQ ID NO: 1127), AACguaagcu (SEQ ID NO: 4179), CAGgucugca (SEQ ID NO: 4180), CAGguauugu (SEQ ID NO: 1267), GUGguaauuc (SEQ ID NO: 2356), GAGguauaug (SEQ ID NO: 1951), GCCgugagcc (SEQ ID NO: 4181), GAGguaagag (SEQ ID NO: 1883), UGAguaugua (SEQ ID NO: 2787), CAGguaaggg (SEQ ID NO: 1145), GAGguaaauu (SEQ ID NO: 1876), CAGgcaacuu (SEQ ID NO: 4182), UGUguaaguc (SEQ ID NO: 2908), CAGgugcgcu (SEQ ID NO: 4183), CGGguaaacc (SEQ ID NO: 4184), CCGgucaguc (SEQ ID NO: 4185), UAGgugggcg (SEQ ID NO: 4186), GCGgucaguu (SEQ ID NO: 4187), GGGguggguc (SEQ ID NO: 4188), AGCguaauag (SEQ ID NO: 4189), ACGgugaguc (SEQ ID NO: 4190), CUGguacuug (SEQ ID NO: 1722), CAGguuggua (SEQ ID NO: 4191), AGAguaugug (SEQ ID NO: 695), CUGgugggua (SEQ ID NO: 1771), GAGguggcuu (SEQ ID NO: 4192), AUAguauuga (SEQ ID NO: 4193), UGAgucguce (SEQ ID NO: 4194), CAGgugcucu (SEQ ID NO: 4195), UACguaauau (SEQ ID NO: 4196), GCUguccuga (SEQ ID NO: 4197), CAGgcugcac (SEQ ID NO: 4198), CUGgugcgcu (SEQ ID NO: 1766), GCGguaagaa (SEQ ID NO: 4199), UAAguuacuu (SEQ ID NO: 4200), GAAgugagug (SEQ ID NO: 1840), UAGgcaaguc (SEQ ID NO: 2460), UAAguaaaua (SEQ ID NO: 4201), ACGgugagug (SEQ ID NO: 607), CAGguagguu (SEQ ID NO: 1223), GGGguauaac (SEQ ID NO: 4202), GUUgugaguu (SEQ ID NO: 2410), CAUgugagua (SEQ ID NO: 1539), GAGgugcauu (SEQ ID NO: 4203), AAGguuugua (SEQ ID NO: 466), UCGguaaugu (SEQ ID NO: 4204), CGAguaaggg (SEQ ID NO: 1616), GAGgcacgga (SEQ ID NO: 4205), AGGgugugga (SEQ ID NO: 4206), CAGguauggu (SEQ ID NO: 1257), AAGguagaaa (SEQ ID NO: 203), CAGgugccug (SEQ ID NO: 1373), UGGguauaug (SEQ ID NO: 4207), UGAgugagac (SEQ ID NO: 4208), UGGguaauuu (SEQ ID NO: 2847), AUGguaaaua (SEQ ID NO: 881), AAGgcaaagg (SEQ ID NO: 4209), AGUguuuguu (SEQ ID NO: 4210), AUGguauugg (SEQ ID NO: 4211), CUGgugagge (SEQ ID NO: 1756), UUGguaaaau (SEQ ID NO: 2948), ACAgugaguu (SEQ ID NO: 563), CAGgugcugu (SEQ ID NO: 4212), GAGguuaaga (SEQ ID NO: 2080), AGAguaagaa (SEQ ID NO: 659), GAGguccgcg (SEQ ID NO: 4213), GUGgugagga (SEQ ID NO: 2382), CAGgugagcc (SEQ ID NO: 1338), CAGgugacau (SEQ ID NO: 1324), AUGgcaagcu (SEQ ID NO: 4214), UCGguaauau (SEQ ID NO: 4215), CAGgcaacaa (SEQ ID NO: 4216), GGGguaggga (SEQ ID NO: 2257), CUGgucucgc (SEQ ID NO: 4217), UAGguaacga (SEQ ID NO: 4218), CGGguaaggu (SEQ ID NO: 4219), UAGguaaugc (SEQ ID NO: 4220), CAGgcaagaa (SEQ ID NO: 1099), ACAguaggua (SEQ ID NO: 4221), CAAguaugag (SEQ ID NO: 1049), GCUguucgaa (SEQ ID NO: 4222), AAGguuaugc (SEQ ID NO: 4223), GAUgugaguu (SEQ ID NO: 2136), CAGguggaga (SEQ ID NO: 1396), AGAguuaguu (SEQ ID NO: 4224), UGAgugugcg (SEQ ID NO: 4225), GAGguacagc (SEQ ID NO: 1907), CAGguaagac (SEQ ID NO: 1139), CAUgugcuuu (SEQ ID NO: 4226), AGGguguguu (SEQ ID NO: 4227), ACAguuaagg (SEQ ID NO: 4228), ACAgugaggg (SEQ ID NO: 4229), GAUguauacc (SEQ ID NO: 4230), UUAguaagcu (SEQ ID NO: 4231), CAGguaagau (SEQ ID NO: 1141), AGAgcugcgu (SEQ ID NO: 4232), GAGgcaaguu (SEQ ID NO: 1860), GAAguaagug (SEQ ID NO: 1819), AAGgugaaaa (SEQ ID NO: 4233), AAGguaccua (SEQ ID NO: 4234), GAGguaucag (SEQ ID NO: 4235), AUGguaugua (SEQ ID NO: 4236), AAGguaugaa (SEQ ID NO: 253), UUGgugagcc (SEQ ID NO: 4237), AAGguuagga (SEQ ID NO: 420), AGGguaugua (SEQ ID NO: 768), CAGguaccga (SEQ ID NO: 4238), AGAguaaacu (SEQ ID NO: 4239), AAGgugcaua (SEQ ID NO: 4240), AAGguaaugu (SEQ ID NO: 167), CCGgugugug (SEQ ID NO: 4241), AGGguaaauu (SEQ ID NO: 729), GGGguuuggc (SEQ ID NO: 4242), CAGguacacg (SEQ ID NO: 1164), UUGguaacca (SEQ ID NO: 4243), GAGgucaggu (SEQ ID NO: 1986), UCUguuggua (SEQ ID NO: 4244), CAGguuaguu (SEQ ID NO: 1458), UUGguauguc (SEQ ID NO: 4245), AAGgugcguc (SEQ ID NO: 4246), AGGguaagaa (SEQ ID NO: 733), UUUguaagcc (SEQ ID NO: 4247), AAGgucaggu (SEQ ID NO: 292), CUGguaaacu (SEQ ID NO: 4248), UCGguaauuu (SEQ ID NO: 4249), CUGguaggcu (SEQ ID NO: 4250), GAGgucugua (SEQ ID NO: 4251), GAGguacuuu (SEQ ID NO: 1922), CUGguaaagg (SEQ ID NO: 4252), CGGgugugug (SEQ ID NO: 1650), CAGguguggu (SEQ ID NO: 4253), UCGguacguc (SEQ ID NO: 4254), CAGgugccag (SEQ ID NO: 4255), GGGgugagaa (SEQ ID NO: 2275), ACAgcuagua (SEQ ID NO: 4256), AAGguauagc (SEQ ID NO: 4257), CUGguaggag (SEQ ID NO: 4258), GCUguacgua (SEQ ID NO: 4259), AAGguaaagg (SEQ ID NO: 128), CAAgcacgag (SEQ ID NO: 4260), CUAguaagac (SEQ ID NO: 4261), CCCguaagcg (SEQ ID NO: 4262), CAAgugugag (SEQ ID NO: 1078), AUGguaaggg (SEQ ID NO: 897), AAGgugaggg (SEQ ID NO: 345), CAAguaggua (SEQ ID NO: 1041), GGUguugcug (SEQ ID NO: 2321), GAGguacugu (SEQ ID NO: 1920), UAGguaagau (SEQ ID NO: 2484), CAGgugcgaa (SEQ ID NO: 1374), GAGguccagg (SEQ ID NO: 4263), UUGguauaca (SEQ ID NO: 2982), GGAgugagua (SEQ ID NO: 2226), GAGgugagau (SEQ ID NO: 2017), AAGguggggc (SEQ ID NO: 4264), CAGguaaacg (SEQ ID NO: 4265), UCGguaacuu (SEQ ID NO: 4266), CAGguaaauu (SEQ ID NO: 1128), GAGgugcgca (SEQ ID NO: 4267), ACUgugagua (SEQ ID NO: 643), ACGgugugac (SEQ ID NO: 4268), GUGguaaguc (SEQ ID NO: 2352), CAGguaggca (SEQ ID NO: 1215), CAGgucagca (SEQ ID NO: 1277), GUGguaugug (SEQ ID NO: 4269), AAAguaucug (SEQ ID NO: 4270), CGGguaugua (SEQ ID NO: 4271), AAGguaauaa (SEQ ID NO: 157), GAGgugggga (SEQ ID NO: 2060), GCUguaggug (SEQ ID NO: 2197), GAAgugaguu (SEQ ID NO: 1841), AAAguauuua (SEQ ID NO: 4272), UAUguaagua (SEQ ID NO: 2653), ACGguaugag (SEQ ID NO: 4273), CUGgugagug (SEQ ID NO: 1761), AGAguaaaau (SEQ ID NO: 4274), GCUguauggc (SEQ ID NO: 4275), AUGguaaacc (SEQ ID NO: 879), GCAguaauaa (SEQ ID NO: 4276), UAAguauuua (SEQ ID NO: 4277), AAUgucagug (SEQ ID NO: 515), AUUgcaggag (SEQ ID NO: 4278), CCGguaagaa (SEQ ID NO: 4279), AAGgcaaguu (SEQ ID NO: 101), GAGguuuguc (SEQ ID NO: 4280), AAGguaacug (SEQ ID NO: 139), AAAguaugag (SEQ ID NO: 4281), GAUguuagua (SEQ ID NO: 4282), CAGguggguc (SEQ ID NO: 1414), AAGguaccga (SEQ ID NO: 4283), CCAguaauua (SEQ ID NO: 4284), GUGguaugcg (SEQ ID NO: 4285), AUGgugcgcu (SEQ ID NO: 4286), CAGgucuaug (SEQ ID NO: 4287), AAGguauuua (SEQ ID NO: 274), CUAguaagau (SEQ ID NO: 4288), AGAguaauuu (SEQ ID NO: 675), GAGguaacgu (SEQ ID NO: 4289), AAGguagcca (SEQ ID NO: 212), CUGgucccgg (SEQ ID NO: 4290), GAGguccuuc (SEQ ID NO: 4291), ACGgucaccc (SEQ ID NO: 4292), AAGguaauac (SEQ ID NO: 158), CAGgugcaug (SEQ ID NO: 1367), AUGguaauag (SEQ ID NO: 4293), UUUguaacac (SEQ ID NO: 4294), UGGguaugau (SEQ ID NO: 4295), CAGgcccccc (SEQ ID NO: 4296), AGAguaguaa (SEQ ID NO: 4297), AGUguaagaa (SEQ ID NO: 814), GAAguauguu (SEQ ID NO: 1833), CAGgugugca (SEQ ID NO: 1434), UUGgugaggg (SEQ ID NO: 3003), UGGguugguu (SEQ ID NO: 4298), CAGguacgua (SEQ ID NO: 1184), GAGgugcggc (SEQ ID NO: 4299), UCUguacggg (SEQ ID NO: 4300), CGGgugcgug (SEQ ID NO: 4301), UACguaagug (SEQ ID NO: 2455), CAUguaagga (SEQ ID NO: 4302), CAGgugacgg (SEQ ID NO: 1329), GAUguaugcu (SEQ ID NO: 4303), UCUgcaauuc (SEQ ID NO: 4304), UGAguaaggc (SEQ ID NO: 2770), GAGguauauu (SEQ ID NO: 1952), AGAgugaguu (SEQ ID NO: 707), AAGguaagcu (SEQ ID NO: 148), UAGgugaagu (SEQ ID NO: 2580), CAGguuagua (SEQ ID NO: 1455), UAUguaagug (SEQ ID NO: 2655), UUGguggggg (SEQ ID NO: 4305), UGAgcucaaa (SEQ ID NO: 4306), UCGguaugua (SEQ ID NO: 4307), UAAguaugcc (SEQ ID NO: 4308), AAUguaagua (SEQ ID NO: 489), CAGguuugca (SEQ ID NO: 4309), ACGgugagag (SEQ ID NO: 4310), CAGguguuuu (SEQ ID NO: 4311), GUGgugagcc (SEQ ID NO: 4312), AGGguacaua (SEQ ID NO: 4313), UAGguaaccc (SEQ ID NO: 4314), GUGgucagua (SEQ ID NO: 4315), CUGgugagcc (SEQ ID NO: 4316), CAGgugcuua (SEQ ID NO: 1390), AUAgucguga (SEQ ID NO: 4317), AUAgugagug (SEQ ID NO: 862), GAGgucaaaa (SEQ ID NO: 4318), CGUguagcuu (SEQ ID NO: 4319), CAGguguuug (SEQ ID NO: 4320), CAGguuggac (SEQ ID NO: 4321), CAGguaagcu (SEQ ID NO: 4322), AGGgucagaa (SEQ ID NO: 4323), CACguauguc (SEQ ID NO: 4324), CACgugagug (SEQ ID NO: 1098), GGGguacgga (SEQ ID NO: 4325), AAGgcaggac (SEQ ID NO: 4326), GAGgugaagc (SEQ ID NO: 4327), GAGguuugaa (SEQ ID NO: 4328), CAGguaagug (SEQ ID NO: 1148), CAGguaacca (SEQ ID NO: 1131), CAGguacucc (SEQ ID NO: 1189), AAGgugcuuu (SEQ ID NO: 371), GAGguaaaua (SEQ ID NO: 1873), GAGgcaggug (SEQ ID NO: 4329), GAGguucgga (SEQ ID NO: 4330), CAGguauuug (SEQ ID NO: 1270), CAGguaaaua (SEQ ID NO: 1125), CAGgugaugu (SEQ ID NO: 1354), CAGgugauac (SEQ ID NO: 4331), GAGgugaggc (SEQ ID NO: 2023), AGGguggggg (SEQ ID NO: 4332), UAAguaaguu (SEQ ID NO: 2425), UGGgugaaca (SEQ ID NO: 4333), UAGguacugc (SEQ ID NO: 4334), CAGgcuccug (SEQ ID NO: 4335), AGGguaggca (SEQ ID NO: 753), CAGgugcccg (SEQ ID NO: 1371), GAGguacauc (SEQ ID NO: 4336), AGGgugugug (SEQ ID NO: 804), AAGguaguaa (SEQ ID NO: 231), UGGguaugag (SEQ ID NO: 2859), GGGgugugug (SEQ ID NO: 2294), CUAguaggug (SEQ ID NO: 4337), GAGgcaagga (SEQ ID NO: 4338), AAGgcaagac (SEQ ID NO: 4339), AAAgugcggu (SEQ ID NO: 4340), AAGguugguu (SEQ ID NO: 450), GAGguuaaug (SEQ ID NO: 4341), UUGgugaguc (SEQ ID NO: 3005), UCGguuagcu (SEQ ID NO: 2738), GCAguaagca (SEQ ID NO: 4342), AAGgcaagca (SEQ ID NO: 4343), ACAguaagcu (SEQ ID NO: 4344), GAGguaacag (SEQ ID NO: 1878), AAAguacgua (SEQ ID NO: 4345), GAGguaauac (SEQ ID NO: 1896), UUGguaggug (SEQ ID NO: 2980), CUGguuaguc (SEQ ID NO: 4346), GAGgugacgc (SEQ ID NO: 4347), ACAguaagga (SEQ ID NO: 4348), AAUguacuua (SEQ ID NO: 4349), GGGguacagu (SEQ ID NO: 4350), CGUguaugug (SEQ ID NO: 4351), UCCguagguu (SEQ ID NO: 4352), GAGguggucg (SEQ ID NO: 4353), UCAgugaguc (SEQ ID NO: 4354), AAAguaagca (SEQ ID NO: 15), GAGgucuggu (SEQ ID NO: 1999), GAGguaauua (SEQ ID NO: 4355), GUAguaagua (SEQ ID NO: 2323), AAGgugggga (SEQ ID NO: 382), UCUgugagca (SEQ ID NO: 4356), GAAguucgug (SEQ ID NO: 4357), ACGgugaggc (SEQ ID NO: 4358), UCAgugagua (SEQ ID NO: 2699), UAGguaguug (SEQ ID NO: 4359), GGUgucuggg (SEQ ID NO: 4360), GGGguaagug (SEQ ID NO: 2252), GAGguggguu (SEQ ID NO: 2066), UGUgugaguu (SEQ ID NO: 4361), CAUguaagua (SEQ ID NO: 1522), AAGguaggug (SEQ ID NO: 229), AAUguaggag (SEQ ID NO: 4362), GAGgcacguc (SEQ ID NO: 4363), CAAguacauu (SEQ ID NO: 4364), UUGguacaga (SEQ ID NO: 4365), GAGguaguag (SEQ ID NO: 1941), AAAgugaggg (SEQ ID NO: 57), UUGgucagug (SEQ ID NO: 4366), AGGgugaguc (SEQ ID NO: 796), CAGgugaaca (SEQ ID NO: 1317), GGUgugggcc (SEQ ID NO: 4367), CGGgugagcu (SEQ ID NO: 4368), GGGgugaguc (SEQ ID NO: 2283), ACAgugagag (SEQ ID NO: 4369), AGGgugaggu (SEQ ID NO: 794), GCUguaaguc (SEQ ID NO: 2194), AUAguagguu (SEQ ID NO: 4370), CAGgcaugug (SEQ ID NO: 1114), AAGguaaguu (SEQ ID NO: 156), CAGguccgug (SEQ ID NO: 4371), GAGgcaggua (SEQ ID NO: 4372), AUGguggaag (SEQ ID NO: 4373), AUGgugggcg (SEQ ID NO: 4374), GAGgugagaa (SEQ ID NO: 2014), AGUgugagca (SEQ ID NO: 832), UUGguaagua (SEQ ID NO: 2962), CAAguaagca (SEQ ID NO: 4375), GGUgugagcu (SEQ ID NO: 2313), CCCgugggua (SEQ ID NO: 4376), CAGguagaau (SEQ ID NO: 4377), CAGgcugagc (SEQ ID NO: 4378), CUGguggece (SEQ ID NO: 4379), UGAguaagag (SEQ ID NO: 4380), CACguuagcu (SEQ ID NO: 4381), AAGgugaguc (SEQ ID NO: 348), AAGguagcuc (SEQ ID NO: 4382), UCGgugaguu (SEQ ID NO: 4383), GAGgcccuuc (SEQ ID NO: 4384), CAGguuaugc (SEQ ID NO: 4385), CCUguaagcu (SEQ ID NO: 4386), CAGgucuccu (SEQ ID NO: 4387), UAGguaggcu (SEQ ID NO: 4388), GGGguagggg (SEQ ID NO: 4389), AAGguaguga (SEQ ID NO: 4390), GAGguuguug (SEQ ID NO: 4391), CAGguugguu (SEQ ID NO: 1489), AAAguaagcc (SEQ ID NO: 16), ACAgugagug (SEQ ID NO: 562), UGGgugugau (SEQ ID NO: 4392), CCCguaacua (SEQ ID NO: 4393), AAGguguugc (SEQ ID NO: 408), AAAgcuggug (SEQ ID NO: 4394), GAGguauagu (SEQ ID NO: 4395), ACGguaagag (SEQ ID NO: 4396), AUGguacggu (SEQ ID NO: 913), GAGgccaguu (SEQ ID NO: 4397), GAGguaugcg (SEQ ID NO: 1960), UCGgugggag (SEQ ID NO: 4398), AAGguggaua (SEQ ID NO: 372), CCAguguggc (SEQ ID NO: 4399), AGGguaagug (SEQ ID NO: 742), UCUguagguc (SEQ ID NO: 4400), CAGgcaagga (SEQ ID NO: 1102), CGGguaauuu (SEQ ID NO: 1628), AUUgugaguc (SEQ ID NO: 1010), CAGguaaacc (SEQ ID NO: 1121), AAGgucaauu (SEQ ID NO: 4401), AAGgugaaua (SEQ ID NO: 327), GUCguaagaa (SEQ ID NO: 4402), GCGguaaguc (SEQ ID NO: 4403), CUGguagagc (SEQ ID NO: 4404), GAGgucgguc (SEQ ID NO: 4405), CAGguaaaca (SEQ ID NO: 1120), AAGgcaagga (SEQ ID NO: 98), CAGgucgucu (SEQ ID NO: 4406), GGGguagggc (SEQ ID NO: 4407), CUGguacuaa (SEQ ID NO: 1721), GAGguagcug (SEQ ID NO: 1929), CUUgucagcu (SEQ ID NO: 4408), UAGguaaggc (SEQ ID NO: 2489), CUGguauuac (SEQ ID NO: 4409), UAAguacguc (SEQ ID NO: 4410), AAGguaagcc (SEQ ID NO: 146), ACGgugaaag (SEQ ID NO: 4411), CCAgccaaua (SEQ ID NO: 4412), CAGguuuguc (SEQ ID NO: 4413), AAGguauaau (SEQ ID NO: 239), AAGgucuuag (SEQ ID NO: 4414), AGGgugagcu (SEQ ID NO: 791), AAGguuaggg (SEQ ID NO: 4415), CGGguaaauu (SEQ ID NO: 4416), CAGguaacgg (SEQ ID NO: 4417), AGAgugugua (SEQ ID NO: 4418), ACAguaaguu (SEQ ID NO: 549), GAUguaauuu (SEQ ID NO: 4419), GAGguaggga (SEQ ID NO: 1934), UUGgcaagug (SEQ ID NO: 2945), AAAgugagga (SEQ ID NO: 4420), AAGguagugc (SEQ ID NO: 234), AGAguaauuc (SEQ ID NO: 674), GGAguaaaua (SEQ ID NO: 4421), GUGguaccca (SEQ ID NO: 4422), CAGguauugc (SEQ ID NO: 4423), GAUgugaggg (SEQ ID NO: 4424), CAAguaaauc (SEQ ID NO: 1017), CAGgugucuc (SEQ ID NO: 1428), AAGguaacag (SEQ ID NO: 4425), UUGguaaaag (SEQ ID NO: 4426), CAGguaucau (SEQ ID NO: 1240), ACGgugagac (SEQ ID NO: 4427), CUGguaugac (SEQ ID NO: 4428), CAGguucacu (SEQ ID NO: 4429), GAGgugauca (SEQ ID NO: 4430), AGUguaaguc (SEQ ID NO: 4431), AACguaagua (SEQ ID NO: 4432), AAAgugagug (SEQ ID NO: 60), GAGguacagg (SEQ ID NO: 4433), CAAguaauga (SEQ ID NO: 4434), GAUguaagga (SEQ ID NO: 4435), UCAguucccc (SEQ ID NO: 4436), GCGguaagga (SEQ ID NO: 4437), UAGguacuaa (SEQ ID NO: 4438), AAGgugaaag (SEQ ID NO: 321), ACUguaagug (SEQ ID NO: 4439), UGGguaugug (SEQ ID NO: 2862), AUGguaacag (SEQ ID NO: 884), CAGguagggu (SEQ ID NO: 1219), ACAguaagug (SEQ ID NO: 548), AAGgugcucc (SEQ ID NO: 366), AAGgugugcu (SEQ ID NO: 4440), AAGgugguga (SEQ ID NO: 4441), ACGgugcgcc (SEQ ID NO: 4442), AAGguauugc (SEQ ID NO: 4443), GGGguaugug (SEQ ID NO: 2267), CAGgugggcu (SEQ ID NO: 1408), GAGguauguu (SEQ ID NO: 1968), AACgugaaua (SEQ ID NO: 4444), CAGguaaugg (SEQ ID NO: 1154), UAGguaugau (SEQ ID NO: 4445), CAGgcaggug (SEQ ID NO: 1108), GGGguugguc (SEQ ID NO: 4446), AAGguauggg (SEQ ID NO: 262), UAAgugaggc (SEQ ID NO: 4447), CAAgugaucg (SEQ ID NO: 4448), AAAguacggg (SEQ ID NO: 4449), AGAgcuacag (SEQ ID NO: 4450), GAGgugggaa (SEQ ID NO: 2054), CAGguacuuu (SEQ ID NO: 1195), GAGgugagag (SEQ ID NO: 2016), CAGguagguc (SEQ ID NO: 1221), UGGguacagc (SEQ ID NO: 4451), AAGgugucag (SEQ ID NO: 396), AAGgcaagaa (SEQ ID NO: 4452), GAGguaaaca (SEQ ID NO: 4453), AAGguaaagu (SEQ ID NO: 129), AAGguaguca (SEQ ID NO: 4454), CUGguauguc (SEQ ID NO: 4455), GAGguauggg (SEQ ID NO: 1963), AAGguauugu (SEQ ID NO: 273), CUGguacuga (SEQ ID NO: 4456), GAGguaagcu (SEQ ID NO: 1888), UGGgugggua (SEQ ID NO: 2883), CAGguucgug (SEQ ID NO: 4457), AAGguauggu (SEQ ID NO: 4458), CAGgugagca (SEQ ID NO: 1337), UGGguaaauu (SEQ ID NO: 2827), UGUguaggug (SEQ ID NO: 4459), UGUgugagcc (SEQ ID NO: 2921), CUGguaauau (SEQ ID NO: 4460), AAAguauguu (SEQ ID NO: 45), UGUguaagaa (SEQ ID NO: 2903), CUAgugagaa (SEQ ID NO: 4461), AGGguagguc (SEQ ID NO: 757), AAGgugggug (SEQ ID NO: 385), UCGguaagug (SEQ ID NO: 4462), AGUguaaaua (SEQ ID NO: 812), GAUguaagug (SEQ ID NO: 2122), AAGguuagug (SEQ ID NO: 424), UAGguaagca (SEQ ID NO: 2485), CAAgugagaa (SEQ ID NO: 1061), AGUguaagua (SEQ ID NO: 819), CAGgugaauc (SEQ ID NO: 1321), UGGgugagac (SEQ ID NO: 2868), AAGguagggc (SEQ ID NO: 224), CUGguuugug (SEQ ID NO: 1788), GCGguagggc (SEQ ID NO: 4463), GAGguaaucc (SEQ ID NO: 4464), AUUguaauaa (SEQ ID NO: 4465), CUGgugaaua (SEQ ID NO: 1748), AAGguuuaaa (SEQ ID NO: 4466), CCUguacugu (SEQ ID NO: 4467), GCGgugagcg (SEQ ID NO: 4468), AAGguaaucc (SEQ ID NO: 162), UAUgugagua (SEQ ID NO: 2671), CCCgugagug (SEQ ID NO: 1573), CAGgugcaga (SEQ ID NO: 1363), CAGgucaguu (SEQ ID NO: 1284), CAGguaggcu (SEQ ID NO: 4469), AAAguaagug (SEQ ID NO: 23), UAGguugguc (SEQ ID NO: 4470), CAGguugccu (SEQ ID NO: 4471), AAGguaugga (SEQ ID NO: 260), GGUguggacg (SEQ ID NO: 4472), AAAgugagaa (SEQ ID NO: 51), AGGgugagag (SEQ ID NO: 788), GAUguggcau (SEQ ID NO: 4473), UCGguaaggu (SEQ ID NO: 4474), GAGgugcguc (SEQ ID NO: 4475), CGGgugaguc (SEQ ID NO: 4476), AAGguacggg (SEQ ID NO: 190), GAGguucuug (SEQ ID NO: 4477), AAGgugcuug (SEQ ID NO: 4478), UAGguaugua (SEQ ID NO: 2551), AUGgucagca (SEQ ID NO: 4479), CGGguacuca (SEQ ID NO: 4480), AGGgugagga (SEQ ID NO: 792), AUCgugagua (SEQ ID NO: 869), UCAguaagua (SEQ ID NO: 2689), UAGguaaaua (SEQ ID NO: 2469), AAGguaauug (SEQ ID NO: 170), GAAgucagug (SEQ ID NO: 1835), CAGguacaaa (SEQ ID NO: 1160), AAAguuaauc (SEQ ID NO: 4481), AGCgugagcg (SEQ ID NO: 4482), CCGgcuggug (SEQ ID NO: 4483), AGUguaauuu (SEQ ID NO: 4484), UGAgccacuc (SEQ ID NO: 4485), GGGgucugua (SEQ ID NO: 4486), AUGgcauguc (SEQ ID NO: 4487), CGGguaaaga (SEQ ID NO: 4488), AGGguagcau (SEQ ID NO: 4489), CGGguaggag (SEQ ID NO: 1631), GAGguucgug (SEQ ID NO: 4490), UAAguuauuc (SEQ ID NO: 4491), UAUguaagau (SEQ ID NO: 2650), AAGguaguuu (SEQ ID NO: 237), CAGgugguau (SEQ ID NO: 4492), GUGguaauga (SEQ ID NO: 2355), AAGgugauuu (SEQ ID NO: 359), CAGgugaagu (SEQ ID NO: 4493), GUAguaauua (SEQ ID NO: 4494), AUGguuggug (SEQ ID NO: 4495), CCAguaagug (SEQ ID NO: 1557), UAGgugagag (SEQ ID NO: 2589), AUGgugaggc (SEQ ID NO: 959), AAAguuagug (SEQ ID NO: 72), AAGgugccuu (SEQ ID NO: 4496), UAGguaugag (SEQ ID NO: 2546), CAGgugugac (SEQ ID NO: 1431), CUGguggguu (SEQ ID NO: 1774), AUGguaagga (SEQ ID NO: 896), UCUguaagaa (SEQ ID NO: 2740), UCCgugaguu (SEQ ID NO: 4497), AAAgcaggua (SEQ ID NO: 4498), UAUgugagug (SEQ ID NO: 2672), CAGguggagg (SEQ ID NO: 4499), CAGguuagac (SEQ ID NO: 4500), AUAguaagac (SEQ ID NO: 846), AAGguguugu (SEQ ID NO: 4501), GAGgucugug (SEQ ID NO: 4502), AAGguaagau (SEQ ID NO: 144), CAUguaaguu (SEQ ID NO: 1524), CUGguaauua (SEQ ID NO: 4503), CAGguaggcg (SEQ ID NO: 4504), AGAguaaguc (SEQ ID NO: 669), UGGgugagga (SEQ ID NO: 2872), AAUguaggua (SEQ ID NO: 4505), UAGguuagca (SEQ ID NO: 4506), GGGguaggua (SEQ ID NO: 2258), GAGguauugc (SEQ ID NO: 4507), AUUguacaca (SEQ ID NO: 4508), GAAguaggua (SEQ ID NO: 4509), GGAguaagcu (SEQ ID NO: 2212), UAGguaugug (SEQ ID NO: 2553), GAGgugaaua (SEQ ID NO: 2007), GAGgugggau (SEQ ID NO: 2056), AAGguaaucu (SEQ ID NO: 163), GGUgugaguu (SEQ ID NO: 4510), AACgugaguu (SEQ ID NO: 4511), GAGguaaccg (SEQ ID NO: 4512), UAGguaagga (SEQ ID NO: 2488), AUUguaagaa (SEQ ID NO: 4513), UGGgugagca (SEQ ID NO: 2870), AAGguaaggc (SEQ ID NO: 150), CCAguaucgu (SEQ ID NO: 4514), CCGgugggug (SEQ ID NO: 4515), GAGguagugu (SEQ ID NO: 4516), ACGgugggaa (SEQ ID NO: 4517), GAGgugaccu (SEQ ID NO: 2011), CACguaugua (SEQ ID NO: 4518), AGGgugggga (SEQ ID NO: 799), AAUguaaguc (SEQ ID NO: 490), AAAguuaagu (SEQ ID NO: 70), CAUgugagug (SEQ ID NO: 1541), AGAguauguc (SEQ ID NO: 694), GCGguaugac (SEQ ID NO: 4519), CGGgugaguu (SEQ ID NO: 1643), CCGguauuuu (SEQ ID NO: 4520), GAGguagaac (SEQ ID NO: 4521), UAGguaugaa (SEQ ID NO: 2545), CAGgcgcgug (SEQ ID NO: 4522), CAAguaaguc (SEQ ID NO: 1027), AGUguaagau (SEQ ID NO: 816), AAGguucuac (SEQ ID NO: 4523), CCAguaagua (SEQ ID NO: 1555), GAGguagcag (SEQ ID NO: 4524), CAGgucuguu (SEQ ID NO: 1312), CAGguacaau (SEQ ID NO: 1162), CCGguaaaga (SEQ ID NO: 1574), UAAgugcugu (SEQ ID NO: 4525), AGGgugagaa (SEQ ID NO: 786), CUCguaaggu (SEQ ID NO: 4526), CAGgucagcu (SEQ ID NO: 4527), CAGguaaggc (SEQ ID NO: 1144), AGGgugcagg (SEQ ID NO: 4528), GAGgugaaac (SEQ ID NO: 4529), AGGguaagua (SEQ ID NO: 740), AAUguaugcc (SEQ ID NO: 4530), AAGguaagca (SEQ ID NO: 145), ACGguacggu (SEQ ID NO: 587), AAGguaauga (SEQ ID NO: 164), UCUgcucaau (SEQ ID NO: 4531), ACGguaaugu (SEQ ID NO: 4532), AAGguaguug (SEQ ID NO: 4533), ACGguaagug (SEQ ID NO: 580), CAGgugauga (SEQ ID NO: 4534), GAGguaacac (SEQ ID NO: 4535), GAGguaggua (SEQ ID NO: 1937), CAGguaccuu (SEQ ID NO: 1179), CAGguaauaa (SEQ ID NO: 1150), UUGgugggug (SEQ ID NO: 3016), CUGguaauga (SEQ ID NO: 1710), UAGguaaguc (SEQ ID NO: 2492), AGGgugugac (SEQ ID NO: 4536), GAGgcaauaa (SEQ ID NO: 4537), GUGguaaagc (SEQ ID NO: 4538), CUGgugggcg (SEQ ID NO: 4539), GAUguauguu (SEQ ID NO: 2128), AGGgugagac (SEQ ID NO: 787), UCGgucagca (SEQ ID NO: 4540), AUGgugauua (SEQ ID NO: 4541), CGAgugugua (SEQ ID NO: 4542), CAGguuggug (SEQ ID NO: 1488), AGCgcaagua (SEQ ID NO: 4543), UGGguacguu (SEQ ID NO: 4544), GAGguauuug (SEQ ID NO: 1974), AGUguacaua (SEQ ID NO: 4545), AUGguaagua (SEQ ID NO: 898), ACAguagguu (SEQ ID NO: 4546), AAGgugagag (SEQ ID NO: 337), UUGgugaagu (SEQ ID NO: 4547), AAAguaugua (SEQ ID NO: 43), UGGguaagga (SEQ ID NO: 4548), UAGgugccuu (SEQ ID NO: 4549), and CCUgugggug (SEQ ID NO: 4550).

Additional exemplary gene sequences and splice site sequences (e.g., 5′ splice site sequences) include UCCguaaguu (SEQ ID NO: 4551), GUGguaaacg (SEQ ID NO: 4552), CGGgugcggu (SEQ ID NO: 4553), CAUguacuuc (SEQ ID NO: 4554), AGAguaaagg (SEQ ID NO: 4555), CGCgugagua (SEQ ID NO: 4556), AGAgugggca (SEQ ID NO: 4557), AGAguaagcc (SEQ ID NO: 4558), AGAguaaaca (SEQ ID NO: 4559), GUGguuauga (SEQ ID NO: 4560), AGGguaauaa (SEQ ID NO: 4561), UGAguaagac (SEQ ID NO: 4562), AGAguuuguu (SEQ ID NO: 4563), CGGgucugca (SEQ ID NO: 4564), CAGguaaguc (SEQ ID NO: 4565), AAGguagaau (SEQ ID NO: 4566), CAGgucccuc (SEQ ID NO: 4567), AGAguaaugg (SEQ ID NO: 4568), GAGgucuaag (SEQ ID NO: 4569), AGAguagagu (SEQ ID NO: 4570), AUGgucagua (SEQ ID NO: 4571), GAGgccuggg (SEQ ID NO: 4572), AAGguguggc (SEQ ID NO: 4573), AGAgugaucu (SEQ ID NO: 4574), AAGguaucca (SEQ ID NO: 4575), UUCguaagua (SEQ ID NO: 4576), UAAgugggug (SEQ ID NO: 4577), GCCgugaacg (SEQ ID NO: 4578), GAGguugugg (SEQ ID NO: 4579), UAUguaugca (SEQ ID NO: 4580), UGUguaacaa (SEQ ID NO: 4581), AGGguauuag (SEQ ID NO: 4582), UGAguauauc (SEQ ID NO: 4583), AGAguuugug (SEQ ID NO: 4584), GAGgucgcug (SEQ ID NO: 4585), GAGgucaucg (SEQ ID NO: 4586), ACGguaaagc (SEQ ID NO: 4587), UGAguacuug (SEQ ID NO: 4588), CGAgucgccg (SEQ ID NO: 4589), CUGguacguc (SEQ ID NO: 4590), AGGguauugc (SEQ ID NO: 4591), GAAgugaaug (SEQ ID NO: 4592), CAGaugaguc (SEQ ID NO: 4593), UGGguauugg (SEQ ID NO: 4594), UGAguaaaga (SEQ ID NO: 4595), GUGguuccug (SEQ ID NO: 4596), UGAgcaagua (SEQ ID NO: 4597), UAUguaagag (SEQ ID NO: 4598), AAGgucuugc (SEQ ID NO: 4599), AAAgcaugug (SEQ ID NO: 4600), AGAguacagu (SEQ ID NO: 4601), GUGguaaucc (SEQ ID NO: 4602), CAGguagagg (SEQ ID NO: 4603), AAGguacaac (SEQ ID NO: 4604), UGGgcagcau (SEQ ID NO: 4605), CCGgucauca (SEQ ID NO: 4606), CCGguuugua (SEQ ID NO: 4607), UGAguaaggg (SEQ ID NO: 4608), GAAguaugua (SEQ ID NO: 4609), GGGguagcuc (SEQ ID NO: 4610), GCUguacaua (SEQ ID NO: 4611), CUGgucucuu (SEQ ID NO: 4612), GUGguaaaug (SEQ ID NO: 4613), AUCguaagug (SEQ ID NO: 4614), GAGgcaugua (SEQ ID NO: 4615), AAGgucuccc (SEQ ID NO: 4616), UGGgugcguu (SEQ ID NO: 4617), UGUguagguu (SEQ ID NO: 4618), GAAgugagca (SEQ ID NO: 4619), GGUguaauuu (SEQ ID NO: 4620), CUGgugaaau (SEQ ID NO: 4621), AUCguaaguc (SEQ ID NO: 4622), AGAguaaucc (SEQ ID NO: 4623), GGAguagguc (SEQ ID NO: 4624), GAGguaccaa (SEQ ID NO: 4625), CUUguaggug (SEQ ID NO: 4626), AAGguauaag (SEQ ID NO: 4627), AGAguuggua (SEQ ID NO: 4628), AUGguuugug (SEQ ID NO: 4629), UGGgucagau (SEQ ID NO: 4630), AGAguaggac (SEQ ID NO: 4631), AGAguagugu (SEQ ID NO: 4632), AGAguaggag (SEQ ID NO: 4633), CAGgucucua (SEQ ID NO: 4634), AAGguggaug (SEQ ID NO: 4635), UGGguaucaa (SEQ ID NO: 4636), GAUguaugga (SEQ ID NO: 4637), AAGguguuuc (SEQ ID NO: 4638), GCAguguaaa (SEQ ID NO: 4639), UUAguaugua (SEQ ID NO: 4640), UCUguaugca (SEQ ID NO: 4641), AAUguaaaau (SEQ ID NO: 4642), AGAguaaauu (SEQ ID NO: 4643), GGGguacuuu (SEQ ID NO: 4644), GAAguuugau (SEQ ID NO: 4645), AAAguagauu (SEQ ID NO: 4646), UGUguagagu (SEQ ID NO: 4647), UGGguaagcg (SEQ ID NO: 4648), CGGguucagg (SEQ ID NO: 4649), AGGguacgac (SEQ ID NO: 4650), UCGguaagaa (SEQ ID NO: 4651), AGGguuggca (SEQ ID NO: 4652), AAAguacagu (SEQ ID NO: 4653), UAAguuaagg (SEQ ID NO: 4654), AUGguaaugu (SEQ ID NO: 4655), GUGguuuuac (SEQ ID NO: 4656), AGAguaacaa (SEQ ID NO: 4657), AAGguagccc (SEQ ID NO: 4658), GCGgugaggc (SEQ ID NO: 4659), AUGguucagc (SEQ ID NO: 4660), AAGguacuua (SEQ ID NO: 4661), AAGguccgug (SEQ ID NO: 4662), UAGguaagcg (SEQ ID NO: 4663), AUGguaccuu (SEQ ID NO: 4664), GCCguggugg (SEQ ID NO: 4665), CUGgugeguc (SEQ ID NO: 4666), CAGguggaaa (SEQ ID NO: 4667), AAAgucugua (SEQ ID NO: 4668), GAGguaaccc (SEQ ID NO: 4669), AGAguauggg (SEQ ID NO: 4670), UAUgccccug (SEQ ID NO: 4671), AAGgugccag (SEQ ID NO: 4672), ACGgugcggc (SEQ ID NO: 4673), AGGguacuga (SEQ ID NO: 4674), AGAguaagcg (SEQ ID NO: 4675), CUGgcaaggg (SEQ ID NO: 4676), CCAgugugug (SEQ ID NO: 4677), GAGguagacg (SEQ ID NO: 4678), CGGgugcggg (SEQ ID NO: 4679), GAUguaagcu (SEQ ID NO: 4680), AUUguauuua (SEQ ID NO: 4681), UGCgugagug (SEQ ID NO: 4682), CUGgucuaua (SEQ ID NO: 4683), GAGgugcuag (SEQ ID NO: 4684), GAGgugccau (SEQ ID NO: 4685), CAGguacguc (SEQ ID NO: 4686), GAGguucagc (SEQ ID NO: 4687), AACguaagaa (SEQ ID NO: 4688), AGAguaguac (SEQ ID NO: 4689), AAGguaacgg (SEQ ID NO: 4690), UAGgugugac (SEQ ID NO: 4691), CCGguaauag (SEQ ID NO: 4692), CAGguaccag (SEQ ID NO: 4693), UUUguaauug (SEQ ID NO: 4694), AAUguacgaa (SEQ ID NO: 4695), CAGguaauga (SEQ ID NO: 4696), AUCgucaagg (SEQ ID NO: 4697), CUGguagaug (SEQ ID NO: 4698), GGGgugcagu (SEQ ID NO: 4699), AGUgugagaa (SEQ ID NO: 4700), GGGguuuuau (SEQ ID NO: 4701), CCUguccccu (SEQ ID NO: 4702), AUUgugaagu (SEQ ID NO: 4703), AAGguaaacg (SEQ ID NO: 4704), UACgucgugg (SEQ ID NO: 4705), AAGgugccau (SEQ ID NO: 4706), GGGgucccag (SEQ ID NO: 4707), UAUguauggu (SEQ ID NO: 4708), CGGguaauua (SEQ ID NO: 4709), CGGguacucc (SEQ ID NO: 4710), CAGgugacuu (SEQ ID NO: 4711), AGUguggguu (SEQ ID NO: 4712), AGAguauggc (SEQ ID NO: 4713), AAGgccaaca (SEQ ID NO: 4714), AAAgcaagua (SEQ ID NO: 4715), UCAguagguc (SEQ ID NO: 4716), GUGguggcgg (SEQ ID NO: 4717), CAUguauccu (SEQ ID NO: 4718), UCGgugagcc (SEQ ID NO: 4719), AUAguugggu (SEQ ID NO: 4720), AAUguuagcu (SEQ ID NO: 4721), AUGgugaaug (SEQ ID NO: 4722), CGGguaaugu (SEQ ID NO: 4723), UCUguaggug (SEQ ID NO: 4724), CCGgugaggc (SEQ ID NO: 4725), UGAguccacu (SEQ ID NO: 4726), CUAguaagag (SEQ ID NO: 4727), CGGguggggc (SEQ ID NO: 4728), CGAguaagca (SEQ ID NO: 4729), UGUgccaauu (SEQ ID NO: 4730), UCGguaagcc (SEQ ID NO: 4731), UAUguaggug (SEQ ID NO: 4732), UUGgugggcc (SEQ ID NO: 4733), GAGgcugggc (SEQ ID NO: 4734), AGAguaacuu (SEQ ID NO: 4735), ACGguagguc (SEQ ID NO: 4736), CAGgcccaga (SEQ ID NO: 4737), CCGguggguu (SEQ ID NO: 4738), AAGgugacgg (SEQ ID NO: 4739), GGGguacagc (SEQ ID NO: 4740), CAUguaaguc (SEQ ID NO: 4741), AUUgugagaa (SEQ ID NO: 4742), UGUguaagga (SEQ ID NO: 4743), UUUguaagau (SEQ ID NO: 4744), AGGgucauuu (SEQ ID NO: 4745), UGGguuuguu (SEQ ID NO: 4746), CGAguaagcc (SEQ ID NO: 4747), GUGgugugua (SEQ ID NO: 4748), AUGguauaac (SEQ ID NO: 4749), UGGguacgua (SEQ ID NO: 4750), AAAguagagu (SEQ ID NO: 4751), UCGguaacug (SEQ ID NO: 4752), AGAguaauga (SEQ ID NO: 4753), AUGguggguc (SEQ ID NO: 4754), AGAguaauau (SEQ ID NO: 4755), CAGguacugg (SEQ ID NO: 4756), UAAgucaguu (SEQ ID NO: 4757), GCGguagaga (SEQ ID NO: 4758), AAGgugaugg (SEQ ID NO: 4759), ACAguauguu (SEQ ID NO: 4760), GAUguacguc (SEQ ID NO: 4761), UAGguuucuc (SEQ ID NO: 4762), GAGgcauggg (SEQ ID NO: 4763), AUAgcuaagu (SEQ ID NO: 4764), GUAgucugua (SEQ ID NO: 4765), AAGgugaacg (SEQ ID NO: 4766), GUGguggucg (SEQ ID NO: 4767), GAGguugauc (SEQ ID NO: 4768), UGAguggguu (SEQ ID NO: 4769), ACUguacgug (SEQ ID NO: 4770), CUGgugacug (SEQ ID NO: 4771), CAAguuaagc (SEQ ID NO: 4772), GAGguaccca (SEQ ID NO: 4773), AACguaacuu (SEQ ID NO: 4774), CAGguuacua (SEQ ID NO: 4775), AGAguuaguc (SEQ ID NO: 4776), UGGgcacguc (SEQ ID NO: 4777), AGUguauggu (SEQ ID NO: 4778), AAGguugcaa (SEQ ID NO: 4779), CAGguuguua (SEQ ID NO: 4780), AAGgcauccc (SEQ ID NO: 4781), GAUguaaggc (SEQ ID NO: 4782), AGGguacggg (SEQ ID NO: 4783), GAGgucaaag (SEQ ID NO: 4784), CAAgugagcg (SEQ ID NO: 4785), AGAguaaucu (SEQ ID NO: 4786), UCGguagcug (SEQ ID NO: 4787), AAAguaguag (SEQ ID NO: 4788), CAGguucguc (SEQ ID NO: 4789), CGUguaugaa (SEQ ID NO: 4790), AGUguaaaaa (SEQ ID NO: 4791), AAGgucucac (SEQ ID NO: 4792), UAGguggagc (SEQ ID NO: 4793), UGAguaggug (SEQ ID NO: 4794), AGAguaugcc (SEQ ID NO: 4795), GAGguugcau (SEQ ID NO: 4796), CAAguaagag (SEQ ID NO: 4797), UCUgugugcc (SEQ ID NO: 4798), GAGgugaugc (SEQ ID NO: 4799), GGGgugauaa (SEQ ID NO: 4800), CCCgugagcc (SEQ ID NO: 4801), AGAguaacug (SEQ ID NO: 4802), GCGguaagua (SEQ ID NO: 4803), AGAguacauc (SEQ ID NO: 4804), UCGgucuggg (SEQ ID NO: 4805), UAAguaucuc (SEQ ID NO: 4806), GGCguagguu (SEQ ID NO: 4807), AGAguacgcc (SEQ ID NO: 4808), GAUgucuucu (SEQ ID NO: 4809), AGGgcaaggu (SEQ ID NO: 4810), CGAguaugau (SEQ ID NO: 4811), AUGguagagu (SEQ ID NO: 4812), CAAguacgag (SEQ ID NO: 4813), UCGguaugau (SEQ ID NO: 4814), CCGguguguu (SEQ ID NO: 4815), AGGgucugug (SEQ ID NO: 4816), GGAguaggcu (SEQ ID NO: 4817), AAGgucuaug (SEQ ID NO: 4818), GCAgugcgug (SEQ ID NO: 4819), UGGgugagaa (SEQ ID NO: 4820), AGGguaaagu (SEQ ID NO: 4821), GAGguaggac (SEQ ID NO: 4822), CUAguaagca (SEQ ID NO: 4823), UUAguaggcu (SEQ ID NO: 4824), CUGgugggau (SEQ ID NO: 4825), CUGguuagua (SEQ ID NO: 4826), AAGguacgug (SEQ ID NO: 4827), CGGgugagau (SEQ ID NO: 4828), AAGgugcaug (SEQ ID NO: 4829), AAUgugggcu (SEQ ID NO: 4830), CAGguugacu (SEQ ID NO: 4831), CAGguuacag (SEQ ID NO: 4832), GCGguaacau (SEQ ID NO: 4833), AUUgucaguc (SEQ ID NO: 4834), CAAguauaca (SEQ ID NO: 4835), GAUgucegcc (SEQ ID NO: 4836), AAGgugcgga (SEQ ID NO: 4837), AACguaagag (SEQ ID NO: 4838), UGGguuggua (SEQ ID NO: 4839), CAAguguaag (SEQ ID NO: 4840), GUGguaacgu (SEQ ID NO: 4841), CUGgugauca (SEQ ID NO: 4842), AGGguggggc (SEQ ID NO: 4843), UCGguaaaga (SEQ ID NO: 4844), CAGguacacc (SEQ ID NO: 4845), CGGguaaggg (SEQ ID NO: 4846), CAAguuugcu (SEQ ID NO: 4847), ACAgugcgug (SEQ ID NO: 4848), UUGguauggg (SEQ ID NO: 4849), GAGgcucauc (SEQ ID NO: 4850), CUGguaauag (SEQ ID NO: 4851), AUGguggaua (SEQ ID NO: 4852), UCAgugaauu (SEQ ID NO: 4853), AAUguaauua (SEQ ID NO: 4854), GCAgucuaaa (SEQ ID NO: 4855), AAGguauucu (SEQ ID NO: 4856), GAGgucauca (SEQ ID NO: 4857), UGGguccaug (SEQ ID NO: 4858), AGAguuugua (SEQ ID NO: 4859), AGGguagacu (SEQ ID NO: 4860), AAGguaggac (SEQ ID NO: 4861), UGUguguuga (SEQ ID NO: 4862), UCAguacgug (SEQ ID NO: 4863), AUGgucucuc (SEQ ID NO: 4864), UGAguuagua (SEQ ID NO: 4865), UGAguaaagu (SEQ ID NO: 4866), GAGgugaccg (SEQ ID NO: 4867), GAGguauauc (SEQ ID NO: 4868), CAGgugccau (SEQ ID NO: 4869), AGAgugguga (SEQ ID NO: 4870), GUUguaagaa (SEQ ID NO: 4871), AGAguaaaua (SEQ ID NO: 4872), AGGgugaagg (SEQ ID NO: 4873), CUGguagauu (SEQ ID NO: 4874), GAGguucagg (SEQ ID NO: 4875), AGGgucuuca (SEQ ID NO: 4876), CUGguaaccu (SEQ ID NO: 4877), ACAguacuga (SEQ ID NO: 4878), AGAguggguc (SEQ ID NO: 4879), AUGguaugag (SEQ ID NO: 4880), AAGguuauau (SEQ ID NO: 4881), AGAguauagu (SEQ ID NO: 4882), AAAguaugaa (SEQ ID NO: 4883), UAGguggcua (SEQ ID NO: 4884), ACCguauggg (SEQ ID NO: 4885), AAAguauaau (SEQ ID NO: 4886), UUUguauggc (SEQ ID NO: 4887), GGGgucgcgu (SEQ ID NO: 4888), GUGgugguuu (SEQ ID NO: 4889), CAGguuugac (SEQ ID NO: 4890), GGAguaggcg (SEQ ID NO: 4891), GAGguacccu (SEQ ID NO: 4892), AUGgugugca (SEQ ID NO: 4893), GUGguuggug (SEQ ID NO: 4894), AAAguaugcu (SEQ ID NO: 4895), UAAguuacau (SEQ ID NO: 4896), ACAguaugag (SEQ ID NO: 4897), GGAguauguu (SEQ ID NO: 4898), UUUgugagaa (SEQ ID NO: 4899), AAUgugcguu (SEQ ID NO: 4900), CAGguagagu (SEQ ID NO: 4901), AUGguguuaa (SEQ ID NO: 4902), CAUgugeguc (SEQ ID NO: 4903), AUAguuggau (SEQ ID NO: 4904), GAGguacgua (SEQ ID NO: 4905), GUUgugagaa (SEQ ID NO: 4906), CAAguacauc (SEQ ID NO: 4907), GAGguaguuu (SEQ ID NO: 4908), ACUguacaga (SEQ ID NO: 4909), CCGguuguga (SEQ ID NO: 4910), UGGgucagug (SEQ ID NO: 4911), GUAguaagaa (SEQ ID NO: 4912), GACguacuuu (SEQ ID NO: 4913), AGAgucaguc (SEQ ID NO: 4914), UAGguuaguu (SEQ ID NO: 4915), AGGgcagcag (SEQ ID NO: 4916), AAGguccuac (SEQ ID NO: 4917), AAUguaauug (SEQ ID NO: 4918), CAGgugcggg (SEQ ID NO: 4919), CUGguaaugg (SEQ ID NO: 4920), CAAguagccc (SEQ ID NO: 4921), GAAgucaguu (SEQ ID NO: 4922), ACAguaauug (SEQ ID NO: 4923), UUAguuagua (SEQ ID NO: 4924), CCUguauuuu (SEQ ID NO: 4925), AUCguaagaa (SEQ ID NO: 4926), CCAgugagca (SEQ ID NO: 4927), GAAguaaggc (SEQ ID NO: 4928), UGAgugggua (SEQ ID NO: 4929), UCAgugguag (SEQ ID NO: 4930), UCUguacagg (SEQ ID NO: 4931), CGAgugagug (SEQ ID NO: 4932), UCCguaugug (SEQ ID NO: 4933), CAUgccguuu (SEQ ID NO: 4934), AAAgugacuu (SEQ ID NO: 4935), AGAguaggca (SEQ ID NO: 4936), GAAguaagag (SEQ ID NO: 4937), CAGgcagguu (SEQ ID NO: 4938), UUGguagagc (SEQ ID NO: 4939), AAGguggaaa (SEQ ID NO: 4940), GAGgcagguc (SEQ ID NO: 4941), AUGguacgac (SEQ ID NO: 4942), AGGguaggaa (SEQ ID NO: 4943), AGGguaggua (SEQ ID NO: 4944), UUGguaaggu (SEQ ID NO: 4945), AUGguacaga (SEQ ID NO: 4946), CAGguagagc (SEQ ID NO: 4947), UAGguaaggu (SEQ ID NO: 4948), GGGguuagag (SEQ ID NO: 4949), AAGguaucaa (SEQ ID NO: 4950), GAGguagccc (SEQ ID NO: 4951), CAGgugccuc (SEQ ID NO: 4952), GCAguaagag (SEQ ID NO: 4953), ACGguagagu (SEQ ID NO: 4954), UGGguaaugg (SEQ ID NO: 4955), CUGgucaguu (SEQ ID NO: 4956), GUGguacauu (SEQ ID NO: 4957), AAAguagguu (SEQ ID NO: 4958), AAGgccaaga (SEQ ID NO: 4959), CGGgugggca (SEQ ID NO: 4960), ACGguccggg (SEQ ID NO: 4961), CGAguaugag (SEQ ID NO: 4962), CUGguaugcc (SEQ ID NO: 4963), GAGguggaug (SEQ ID NO: 4964), CAGgccuuuc (SEQ ID NO: 4965), AAAguacauc (SEQ ID NO: 4966), AAAguaauca (SEQ ID NO: 4967), GAGguaacug (SEQ ID NO: 4968), CUGguaaaga (SEQ ID NO: 4969), CGUguaagca (SEQ ID NO: 4970), UGGgcaagua (SEQ ID NO: 4971), GCGguggcga (SEQ ID NO: 4972), GAGguggccg (SEQ ID NO: 4973), AUUgcaugca (SEQ ID NO: 4974), ACGgugacug (SEQ ID NO: 4975), CAGgucagau (SEQ ID NO: 4976), AGAguaacuc (SEQ ID NO: 4977), UGAguaacag (SEQ ID NO: 4978), AAGguacccg (SEQ ID NO: 4979), AGGguaggcu (SEQ ID NO: 4980), GGGgcaggac (SEQ ID NO: 4981), CCUguaagug (SEQ ID NO: 4982), AUUguaagug (SEQ ID NO: 4983), ACUguacgag (SEQ ID NO: 4984), GUAguagugu (SEQ ID NO: 4985), AGAguaugag (SEQ ID NO: 4986), UCAguguggg (SEQ ID NO: 4987), UGGguauaua (SEQ ID NO: 4988), UAGguagcua (SEQ ID NO: 4989), GGGguaaaga (SEQ ID NO: 4990), AGGguuacuu (SEQ ID NO: 4991), CAUguaaaug (SEQ ID NO: 4992), GGAguaguaa (SEQ ID NO: 4993), CAGgucaauc (SEQ ID NO: 4994), CGGguuagug (SEQ ID NO: 4995), UAGguacaug (SEQ ID NO: 4996), UAGguuaaga (SEQ ID NO: 4997), UGGguaccuu (SEQ ID NO: 4998), CGGguggaca (SEQ ID NO: 4999), CAGgucuuac (SEQ ID NO: 5000), AAGguggagc (SEQ ID NO: 5001), AUGguaacca (SEQ ID NO: 5002), UCGguaaguu (SEQ ID NO: 5003), UAUguacaaa (SEQ ID NO: 5004), AAUguagauu (SEQ ID NO: 5005), GUAgcuagua (SEQ ID NO: 5006), AAGguauugg (SEQ ID NO: 5007), GAGgucuuug (SEQ ID NO: 5008), GAAguucagg (SEQ ID NO: 5009), UGGguaucac (SEQ ID NO: 5010), AGAguacugg (SEQ ID NO: 5011), CAGguuaaug (SEQ ID NO: 5012), AGGguacgug (SEQ ID NO: 5013), AGGgcacagg (SEQ ID NO: 5014), CUGguuaguu (SEQ ID NO: 5015), UUGguacgag (SEQ ID NO: 5016), ACGgugauca (SEQ ID NO: 5017), CCUgugagag (SEQ ID NO: 5018), GAGgugaagu (SEQ ID NO: 5019), AAGguacauc (SEQ ID NO: 5020), UCUguaugug (SEQ ID NO: 5021), UUGguggaag (SEQ ID NO: 5022), UGGgcagguu (SEQ ID NO: 5023), GAAguggagc (SEQ ID NO: 5024), ACAguaagac (SEQ ID NO: 5025), CGGguaccaa (SEQ ID NO: 5026), CAAguacguc (SEQ ID NO: 5027), AGAgugaggg (SEQ ID NO: 5028), CGGguaagaa (SEQ ID NO: 5029), AAUguaggug (SEQ ID NO: 5030), AUCgugugcu (SEQ ID NO: 5031), UAGgucaugg (SEQ ID NO: 5032), CAGguuuuga (SEQ ID NO: 5033), AAGgcaugca (SEQ ID NO: 5034), GAGgugcugc (SEQ ID NO: 5035), AAGguuaaua (SEQ ID NO: 5036), CAGguucauc (SEQ ID NO: 5037), GCGguaggug (SEQ ID NO: 5038), GACgugagua (SEQ ID NO: 5039), CAGgucuacu (SEQ ID NO: 5040), UUGguaugag (SEQ ID NO: 5041), AGCgugggca (SEQ ID NO: 5042), AUGguaaggu (SEQ ID NO: 5043), AUGguaccuc (SEQ ID NO: 5044), UUGguauggu (SEQ ID NO: 5045), UAUguaugaa (SEQ ID NO: 5046), UGGguauggg (SEQ ID NO: 5047), GAUguaaaua (SEQ ID NO: 5048), CCGguaaguu (SEQ ID NO: 5049), GAGgucugaa (SEQ ID NO: 5050), GAGgugcgag (SEQ ID NO: 5051), CUGgucagcc (SEQ ID NO: 5052), CAGguuuugu (SEQ ID NO: 5053), CGGguggugu (SEQ ID NO: 5054), UAAguuagua (SEQ ID NO: 5055), UUUgugugug (SEQ ID NO: 5056), CAGguuaacc (SEQ ID NO: 5057), UUGguacuuu (SEQ ID NO: 5058), GCUguaaggc (SEQ ID NO: 5059), AGGguggcug (SEQ ID NO: 5060), GAUguaaaaa (SEQ ID NO: 5061), AAGgucaaaa (SEQ ID NO: 5062), CAGguagcgc (SEQ ID NO: 5063), CAGguuuggc (SEQ ID NO: 5064), GAGgugguuu (SEQ ID NO: 5065), CGGguaaaua (SEQ ID NO: 5066), CUGguucggu (SEQ ID NO: 5067), GGAgugagcc (SEQ ID NO: 5068), AAGgugcgcg (SEQ ID NO: 5069), GAAguacauc (SEQ ID NO: 5070), AGUgucugua (SEQ ID NO: 5071), CCCgugagcu (SEQ ID NO: 5072), GAGguucaca (SEQ ID NO: 5073), CUAgugggua (SEQ ID NO: 5074), GAGguaacua (SEQ ID NO: 5075), UCGguauguc (SEQ ID NO: 5076), UAAguauuug (SEQ ID NO: 5077), CAGguaagcg (SEQ ID NO: 5078), GAGgugguaa (SEQ ID NO: 5079), CGAguaagag (SEQ ID NO: 5080), CCGguaagcu (SEQ ID NO: 5081), GAGgucuugu (SEQ ID NO: 5082), AAGguggguc (SEQ ID NO: 5083), CACguaagug (SEQ ID NO: 5084), AGUguaauga (SEQ ID NO: 5085), AAAgugugua (SEQ ID NO: 5086), GGAgugccaa (SEQ ID NO: 5087), CACgugaguu (SEQ ID NO: 5088), AAGguuggau (SEQ ID NO: 5089), UAUguaaaua (SEQ ID NO: 5090), CUGguaggaa (SEQ ID NO: 5091), UAUguaaacu (SEQ ID NO: 5092), AAUguauuuu (SEQ ID NO: 5093), CUGgcaagug (SEQ ID NO: 5094), UGUgugguau (SEQ ID NO: 5095), UAUguauguu (SEQ ID NO: 5096), UUGgugacuc (SEQ ID NO: 5097), GGAguaaggu (SEQ ID NO: 5098), AAGguagaug (SEQ ID NO: 5099), UGGguagggu (SEQ ID NO: 5100), AAUguaauuc (SEQ ID NO: 5101), GUGguauggc (SEQ ID NO: 5102), GGAguggguu (SEQ ID NO: 5103), AGGguaccac (SEQ ID NO: 5104), UAGgugacag (SEQ ID NO: 5105), ACAguaggca (SEQ ID NO: 5106), AUGguuugaa (SEQ ID NO: 5107), GCAguaacua (SEQ ID NO: 5108), CCGguaggua (SEQ ID NO: 5109), AGAguaggcc (SEQ ID NO: 5110), AAGguugaca (SEQ ID NO: 5111), CUGgugugua (SEQ ID NO: 5112), GAAgucuguc (SEQ ID NO: 5113), UGGgcucgga (SEQ ID NO: 5114), CAGguagccu (SEQ ID NO: 5115), AGAguaggua (SEQ ID NO: 5116), UAAguauguc (SEQ ID NO: 5117), CUGguauauc (SEQ ID NO: 5118), GAGguguguu (SEQ ID NO: 5119), AUGgugcaug (SEQ ID NO: 5120), AAGguacgcc (SEQ ID NO: 5121), UGAguaacua (SEQ ID NO: 5122), GAGgugacag (SEQ ID NO: 5123), GUUguccugu (SEQ ID NO: 5124), UUGgugucuu (SEQ ID NO: 5125), AAUgugaagg (SEQ ID NO: 5126), UUGguggaua (SEQ ID NO: 5127), UAGguguguu (SEQ ID NO: 5128), CUGgcaaguu (SEQ ID NO: 5129), GCAguaagau (SEQ ID NO: 5130), GCGguggaaa (SEQ ID NO: 5131), UGCguccagc (SEQ ID NO: 5132), AAAguggagu (SEQ ID NO: 5133), CGUgugagcc (SEQ ID NO: 5134), AGAguacugu (SEQ ID NO: 5135), CAGguauagc (SEQ ID NO: 5136), UACguaagga (SEQ ID NO: 5137), AAGgucuuua (SEQ ID NO: 5138), AAGguggucu (SEQ ID NO: 5139), GGGguaaauu (SEQ ID NO: 5140), UCAgugagga (SEQ ID NO: 5141), AGAguacguu (SEQ ID NO: 5142), GAGgucguca (SEQ ID NO: 5143), UAGguuugau (SEQ ID NO: 5144), CAUguaaacc (SEQ ID NO: 5145), AAGguggcac (SEQ ID NO: 5146), CAGguagaug (SEQ ID NO: 5147), AACguaaaag (SEQ ID NO: 5148), UAGgucucug (SEQ ID NO: 5149), AUAguaggug (SEQ ID NO: 5150), UAGgcaagag (SEQ ID NO: 5151), UAGgcacggc (SEQ ID NO: 5152), AAGgucuuca (SEQ ID NO: 5153), CCAguaugcu (SEQ ID NO: 5154), CAAgugaguu (SEQ ID NO: 5155), CAGgucucaa (SEQ ID NO: 5156), CAGguuacau (SEQ ID NO: 5157), GGAgugagca (SEQ ID NO: 5158), AGAguacgca (SEQ ID NO: 5159), CUGguguugg (SEQ ID NO: 5160), AAGguacuca (SEQ ID NO: 5161), CUAguaaggg (SEQ ID NO: 5162), AGAguaaaag (SEQ ID NO: 5163), AAGguaacga (SEQ ID NO: 5164), CUGguccccg (SEQ ID NO: 5165), UAAguauggg (SEQ ID NO: 5166), GAGgucgagc (SEQ ID NO: 5167), UUGguauaua (SEQ ID NO: 5168), AAAgucaagg (SEQ ID NO: 5169), AAGgucuagg (SEQ ID NO: 5170), CGAguagguc (SEQ ID NO: 5171), AGGguucguu (SEQ ID NO: 5172), GAGgcaggcc (SEQ ID NO: 5173), CUAguauuac (SEQ ID NO: 5174), ACGguaugug (SEQ ID NO: 5175), UAGgugguuc (SEQ ID NO: 5176), AGAguauaac (SEQ ID NO: 5177), UUGgugcguc (SEQ ID NO: 5178), ACCguuaucu (SEQ ID NO: 5179), CCAgugauga (SEQ ID NO: 5180), GAAguaugca (SEQ ID NO: 5181), GAAguauggc (SEQ ID NO: 5182), CCGguaggac (SEQ ID NO: 5183), AAUguaagca (SEQ ID NO: 5184), AGAguaauug (SEQ ID NO: 5185), AGGguugguu (SEQ ID NO: 5186), GUGguaggag (SEQ ID NO: 5187), AAGgcaguuu (SEQ ID NO: 5188), CAAguaagcc (SEQ ID NO: 5189), CUGgcaagua (SEQ ID NO: 5190), CAGgcaugau (SEQ ID NO: 5191), AGGguaauug (SEQ ID NO: 5192), GGGguaaccu (SEQ ID NO: 5193), AAAguaacua (SEQ ID NO: 5194), UAGgucugcc (SEQ ID NO: 5195), ACGguaugaa (SEQ ID NO: 5196), AGUguauggg (SEQ ID NO: 5197), UGGguuggca (SEQ ID NO: 5198), UAGguaaacu (SEQ ID NO: 5199), AGAgugggua (SEQ ID NO: 5200), AGAguauuug (SEQ ID NO: 5201), AGUguaggaa (SEQ ID NO: 5202), CUUguacgua (SEQ ID NO: 5203), GAUgugagau (SEQ ID NO: 5204), CAGgcagcca (SEQ ID NO: 5205), AAGgucacug (SEQ ID NO: 5206), AAGgucugac (SEQ ID NO: 5207), UAGguuccuu (SEQ ID NO: 5208), CUGgugcuuu (SEQ ID NO: 5209), UGAguuggug (SEQ ID NO: 5210), UUGgugggau (SEQ ID NO: 5211), UGAguagggu (SEQ ID NO: 5212), UCGgugaggu (SEQ ID NO: 5213), AAAguaaaga (SEQ ID NO: 5214), AAGgcaaguc (SEQ ID NO: 5215), CGGguaaagc (SEQ ID NO: 5216), AAAguuaguu (SEQ ID NO: 5217), UUAguaagca (SEQ ID NO: 5218), GAGgucacau (SEQ ID NO: 5219), UAAgugguau (SEQ ID NO: 5220), UAGgugcuuu (SEQ ID NO: 5221), GGAguaggca (SEQ ID NO: 5222), UGAguaagga (SEQ ID NO: 5223), CAGguggagc (SEQ ID NO: 5224), GAUguagaag (SEQ ID NO: 5225), AAUgccugcc (SEQ ID NO: 5226), AUGguaaggc (SEQ ID NO: 5227), UGGguaauau (SEQ ID NO: 5228), CUGguaccuc (SEQ ID NO: 5229), CACgugagcc (SEQ ID NO: 5230), UGAguuugug (SEQ ID NO: 5231), CCGguagugu (SEQ ID NO: 5232), AAAgugacaa (SEQ ID NO: 5233), GAAguggguu (SEQ ID NO: 5234), CAGgugcagc (SEQ ID NO: 5235), GAGgugggcc (SEQ ID NO: 5236), UAUgugcguc (SEQ ID NO: 5237), GGGguacugg (SEQ ID NO: 5238), CUGguagguu (SEQ ID NO: 5239), UUGgcauguu (SEQ ID NO: 5240), AAUguaauac (SEQ ID NO: 5241), UAGgccggug (SEQ ID NO: 5242), AGAgucagua (SEQ ID NO: 5243), UAAguaaauc (SEQ ID NO: 5244), CAGguuccuc (SEQ ID NO: 5245), UAGguacgau (SEQ ID NO: 5246), AGAguuagug (SEQ ID NO: 5247), GCAguaagug (SEQ ID NO: 5248), AGGgugguag (SEQ ID NO: 5249), GGAguaaugu (SEQ ID NO: 5250), GAUguaaguc (SEQ ID NO: 5251), CCAguuucgu (SEQ ID NO: 5252), AAGguucggg (SEQ ID NO: 5253), AUGguggagu (SEQ ID NO: 5254), AAGguaccgg (SEQ ID NO: 5255), GAAgugcgaa (SEQ ID NO: 5256), UGGgucaguu (SEQ ID NO: 5257), AAGguguaga (SEQ ID NO: 5258), UGGguaggcc (SEQ ID NO: 5259), CCAgugaguc (SEQ ID NO: 5260), AAGgucacuu (SEQ ID NO: 5261), AGCgugaggc (SEQ ID NO: 5262), UCCgugguaa (SEQ ID NO: 5263), AGAguacuua (SEQ ID NO: 5264), GGGgucagau (SEQ ID NO: 5265), AAGguggacc (SEQ ID NO: 5266), AGAgugagcg (SEQ ID NO: 5267), AGAgucagau (SEQ ID NO: 5268), UAAguauuac (SEQ ID NO: 5269), AGAguauuuc (SEQ ID NO: 5270), AGAguucagc (SEQ ID NO: 5271), AUGgugaagu (SEQ ID NO: 5272), UAGgugaucc (SEQ ID NO: 5273), GGAguaagau (SEQ ID NO: 5274), UAGguaccaa (SEQ ID NO: 5275), AGAguugguc (SEQ ID NO: 5276), GAAgugagac (SEQ ID NO: 5277), AUCguagguu (SEQ ID NO: 5278), GAGguacgcu (SEQ ID NO: 5279), ACGguaaggg (SEQ ID NO: 5280), CAGgcauguc (SEQ ID NO: 5281), UUAguaagau (SEQ ID NO: 5282), UGAguagguu (SEQ ID NO: 5283), AGGguacgaa (SEQ ID NO: 5284), ACGguauguu (SEQ ID NO: 5285), AGGguacugu (SEQ ID NO: 5286), UUGguaugga (SEQ ID NO: 5287), UAAguaacug (SEQ ID NO: 5288), GCGgucagcc (SEQ ID NO: 5289), UUUgugaguc (SEQ ID NO: 5290), GUGgucagug (SEQ ID NO: 5291), CUGgucugua (SEQ ID NO: 5292), GAGguucuua (SEQ ID NO: 5293), AUGguacuga (SEQ ID NO: 5294), AAUgugcuuu (SEQ ID NO: 5295), AGGguggcgu (SEQ ID NO: 5296), CCGgcaggaa (SEQ ID NO: 5297), CAUguggguc (SEQ ID NO: 5298), UUGguuuguu (SEQ ID NO: 5299), CAGguucugu (SEQ ID NO: 5300), ACGguaagcg (SEQ ID NO: 5301), CUGgucagua (SEQ ID NO: 5302), UCAguaggcu (SEQ ID NO: 5303), UGAguaggac (SEQ ID NO: 5304), CAGguuuuaa (SEQ ID NO: 5305), GAGguguccc (SEQ ID NO: 5306), AGGguggguu (SEQ ID NO: 5307), GUGgugagac (SEQ ID NO: 5308), CACguaggga (SEQ ID NO: 5309), GUGguauuuu (SEQ ID NO: 5310), GAGauauccu (SEQ ID NO: 5311), AAGgugaaca (SEQ ID NO: 5312), UAAguagggc (SEQ ID NO: 5313), CUGgugcggg (SEQ ID NO: 5314), CUGgucaaua (SEQ ID NO: 5315), AGAguaaaaa (SEQ ID NO: 5316), AAGgugcagu (SEQ ID NO: 5317), CGGguaagca (SEQ ID NO: 5318), AAAgugagcc (SEQ ID NO: 5319), AUGguaauca (SEQ ID NO: 5320), GCAguacgug (SEQ ID NO: 5321), AUGguacaug (SEQ ID NO: 5322), AAGguuaaga (SEQ ID NO: 5323), CGGguaaaug (SEQ ID NO: 5324), GAGguucgca (SEQ ID NO: 5325), GAGgcucugg (SEQ ID NO: 5326), AUGgugggac (SEQ ID NO: 5327), AACgugguag (SEQ ID NO: 5328), AAGgugauag (SEQ ID NO: 5329), GGGguuugca (SEQ ID NO: 5330), CAUguaaggg (SEQ ID NO: 5331), UCAguugagu (SEQ ID NO: 5332), AAAgugcggc (SEQ ID NO: 5333), AGAgugagcc (SEQ ID NO: 5334), AUGgcaagaa (SEQ ID NO: 5335), ACAguaaggu (SEQ ID NO: 5336), AAGgucucua (SEQ ID NO: 5337), GUGguaaaaa (SEQ ID NO: 5338), AAAguaggug (SEQ ID NO: 5339), UAGgugcacu (SEQ ID NO: 5340), GUCgugguau (SEQ ID NO: 5341), CAGguauagg (SEQ ID NO: 5342), UGAgugagag (SEQ ID NO: 5343), ACUgugagcc (SEQ ID NO: 5344), AUCguuaguu (SEQ ID NO: 5345), UUUguaccaa (SEQ ID NO: 5346), UGGgugagau (SEQ ID NO: 5347), AGAgugagaa (SEQ ID NO: 5348), AGAguagggg (SEQ ID NO: 5349), AGGgcaagua (SEQ ID NO: 5350), CGGgucagua (SEQ ID NO: 5351), UUGguaugcc (SEQ ID NO: 5352), CGGguuagau (SEQ ID NO: 5353), GGGgugaagu (SEQ ID NO: 5354), CCCgugugaa (SEQ ID NO: 5355), GCAguuugga (SEQ ID NO: 5356), UGCguaagac (SEQ ID NO: 5357), AGAgucugua (SEQ ID NO: 5358), CACgugagca (SEQ ID NO: 5359), AGGguaaaag (SEQ ID NO: 5360), CAGgcugggu (SEQ ID NO: 5361), GAAgucuuca (SEQ ID NO: 5362), AAGgcaaaaa (SEQ ID NO: 5363), GUAguaaaua (SEQ ID NO: 5364), CUAgugagag (SEQ ID NO: 5365), GAAguuucug (SEQ ID NO: 5366), CCUguacgua (SEQ ID NO: 5367), GAGgugcgcg (SEQ ID NO: 5368), AAGguguaaa (SEQ ID NO: 5369), CCAguauguu (SEQ ID NO: 5370), CCGgucagcu (SEQ ID NO: 5371), AUGguuccug (SEQ ID NO: 5372), CAAguuaaau (SEQ ID NO: 5373), AGAguaggcu (SEQ ID NO: 5374), AUGgugggca (SEQ ID NO: 5375), GGAguaagac (SEQ ID NO: 5376), AGGgucacga (SEQ ID NO: 5377), UAGgugauau (SEQ ID NO: 5378), GAAguaaguc (SEQ ID NO: 5379), CGGguaagau (SEQ ID NO: 5380), CAAguagcua (SEQ ID NO: 5381), UGAguaaaau (SEQ ID NO: 5382), GUCguacgug (SEQ ID NO: 5383), AUGguacgua (SEQ ID NO: 5384), CAGgucucgg (SEQ ID NO: 5385), GAGgcauguc (SEQ ID NO: 5386), AGAgugggau (SEQ ID NO: 5387), GUGguuagag (SEQ ID NO: 5388), UGGgugguga (SEQ ID NO: 5389), AAGguuaaac (SEQ ID NO: 5390), CUUguuagcu (SEQ ID NO: 5391), AAAguaggaa (SEQ ID NO: 5392), UAGguuguau (SEQ ID NO: 5393), AGGgugcgcc (SEQ ID NO: 5394), AAGgugggcu (SEQ ID NO: 5395), UAAguaucug (SEQ ID NO: 5396), AAGguaacgu (SEQ ID NO: 5397), AUGguggggc (SEQ ID NO: 5398), CAAguacacg (SEQ ID NO: 5399), GGCguaagug (SEQ ID NO: 5400), AUAguaggac (SEQ ID NO: 5401), AGAgugaggu (SEQ ID NO: 5402), UUUguaaaaa (SEQ ID NO: 5403), GAAguuugua (SEQ ID NO: 5404), CUAguaaucu (SEQ ID NO: 5405), AAGguuuuua (SEQ ID NO: 5406), GAGgugcguu (SEQ ID NO: 5407), UAGgcgagua (SEQ ID NO: 5408), ACCgugagua (SEQ ID NO: 5409), CAGgucccga (SEQ ID NO: 5410), AUGguacugg (SEQ ID NO: 5411), UGAguucagu (SEQ ID NO: 5412), AAUguguggu (SEQ ID NO: 5413), UCCguugguu (SEQ ID NO: 5414), CAGgucagag (SEQ ID NO: 5415), CAGgucccua (SEQ ID NO: 5416), UAGguagacu (SEQ ID NO: 5417), CAAguuaagg (SEQ ID NO: 5418), GAGgugugcg (SEQ ID NO: 5419), GAAgcugccc (SEQ ID NO: 5420), CGAguacgug (SEQ ID NO: 5421), CGGguaggua (SEQ ID NO: 5422), UUGguauuga (SEQ ID NO: 5423), AUUguaugau (SEQ ID NO: 5424), UUGguaugaa (SEQ ID NO: 5425), GAGgugguca (SEQ ID NO: 5426), GCUguaugaa (SEQ ID NO: 5427), CAGguguugc (SEQ ID NO: 5428), CAGguaaaac (SEQ ID NO: 5429), AUAguaaggu (SEQ ID NO: 5430), CUGguuagag (SEQ ID NO: 5431), AGCgugugag (SEQ ID NO: 5432), AAGguuaucu (SEQ ID NO: 5433), CACgugagua (SEQ ID NO: 5434), AGGgucagua (SEQ ID NO: 5435), GAGguauaau (SEQ ID NO: 5436), CAGguuauuu (SEQ ID NO: 5437), AGGguggacu (SEQ ID NO: 5438), AUUguaauuc (SEQ ID NO: 5439), UUUguggguu (SEQ ID NO: 5440), AUGguacgug (SEQ ID NO: 5441), AAGguguucc (SEQ ID NO: 5442), CAGgugacgc (SEQ ID NO: 5443), GAGguacuaa (SEQ ID NO: 5444), ACAguucagu (SEQ ID NO: 5445), GAGgucacgg (SEQ ID NO: 5446), CAAguaaggc (SEQ ID NO: 5447), AAGguuuggg (SEQ ID NO: 5448), AAAgugggcu (SEQ ID NO: 5449), GCGguucuug (SEQ ID NO: 5450), GAGguggagc (SEQ ID NO: 5451), UGAgucagug (SEQ ID NO: 5452), CAGgucaagg (SEQ ID NO: 5453), AGUguaagcu (SEQ ID NO: 5454), GAGgcagaaa (SEQ ID NO: 5455), AAGgucacac (SEQ ID NO: 5456), GAAguagguu (SEQ ID NO: 5457), GUCguaaguu (SEQ ID NO: 5458), AGAguaugca (SEQ ID NO: 5459), CCUgugcaaa (SEQ ID NO: 5460), ACGgugaaaa (SEQ ID NO: 5461), CAGguacgaa (SEQ ID NO: 5462), CAUgugagga (SEQ ID NO: 5463), AGCgugagua (SEQ ID NO: 5464), GGUguguagg (SEQ ID NO: 5465), AACgugagcu (SEQ ID NO: 5466), GAGgugaacu (SEQ ID NO: 5467), AGAguucagu (SEQ ID NO: 5468), AACgugugua (SEQ ID NO: 5469), CAGguugugg (SEQ ID NO: 5470), AAGguacuag (SEQ ID NO: 5471), UCAgugaaaa (SEQ ID NO: 5472), AAUgucuggu (SEQ ID NO: 5473), ACGguaaaau (SEQ ID NO: 5474), CUGguguaag (SEQ ID NO: 5475), GAGgugcgaa (SEQ ID NO: 5476), AGGguuucuc (SEQ ID NO: 5477), CAGguagccc (SEQ ID NO: 5478), AUUguauugg (SEQ ID NO: 5479), AUGguacuua (SEQ ID NO: 5480), GAGgcccgac (SEQ ID NO: 5481), UCGguaagac (SEQ ID NO: 5482), CGGgcuguag (SEQ ID NO: 5483), UAUgugugug (SEQ ID NO: 5484), UAGguagaaa (SEQ ID NO: 5485), GUGgucauua (SEQ ID NO: 5486), UAGgugaaag (SEQ ID NO: 5487), ACUguaauuc (SEQ ID NO: 5488), GCAguacagg (SEQ ID NO: 5489), UCGgugaguc (SEQ ID NO: 5490), UAUguaggga (SEQ ID NO: 5491), AUGguauguc (SEQ ID NO: 5492), GUGgugugug (SEQ ID NO: 5493), CUGgugaccu (SEQ ID NO: 5494), AAUgugaaua (SEQ ID NO: 5495), UAGgucucac (SEQ ID NO: 5496), GAGguuauug (SEQ ID NO: 5497), UGAguaggcu (SEQ ID NO: 5498), CGGgcacgua (SEQ ID NO: 5499), GCAguaaaua (SEQ ID NO: 5500), CCGgugagag (SEQ ID NO: 5501), UAAguugguc (SEQ ID NO: 5502), CCGgugagcc (SEQ ID NO: 5503), AAGguuguca (SEQ ID NO: 5504), CUGguauuau (SEQ ID NO: 5505), GGGguauggg (SEQ ID NO: 5506), AAAgucagua (SEQ ID NO: 5507), UUUguaugua (SEQ ID NO: 5508), UAAguacugc (SEQ ID NO: 5509), CAGguaccaa (SEQ ID NO: 5510), GAAguucaga (SEQ ID NO: 5511), AUGgugcggu (SEQ ID NO: 5512), GUGgugaggu (SEQ ID NO: 5513), UGAguaagcc (SEQ ID NO: 5514), UAUguaaggg (SEQ ID NO: 5515), GUGguggaaa (SEQ ID NO: 5516), GAGgugauug (SEQ ID NO: 5517), GGAguuugua (SEQ ID NO: 5518), AAGgucacga (SEQ ID NO: 5519), GUGguagagg (SEQ ID NO: 5520), UAAguauauc (SEQ ID NO: 5521), AAGgugucca (SEQ ID NO: 5522), UAUgugguau (SEQ ID NO: 5523), GAGguacaau (SEQ ID NO: 5524), AAGguggggg (SEQ ID NO: 5525), GGAguaggug (SEQ ID NO: 5526), and UAGgugacuu (SEQ ID NO: 5527).

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg (SEQ ID NO: 667). In some embodiments, the splice site sequence comprises UGAguaagca (SEQ ID NO: 2768).

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene. In an embodiment, a gene sequence or splice site sequence provided herein is related to a lysosomal storage disease.

In some embodiments, a compound of Formula (I) or (II) described herein interacts with (e.g., binds to) a splicing complex component (e.g., a nucleic acid (e.g., an RNA) or a protein).

In some embodiments, the splicing complex component is selected from 9G8, A1 hnRNP, A2 hnRNP, ASD-1, ASD-2b, ASF, BRR2, B1 hnRNP, C₁ hnRNP, C₂ hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the hnRNP protein comprises A1, A2/B1, L, M, K, U, F, H, G, R, I or C1/C2. Human genes encoding hnRNPs include HNRNPA0, HNRNPA1, HNRNPAIL1, HNRNPAIL2, HNRNPA3, HNRNPA2B1, HNRNPAB, HNRNPB1, HNRNPC, HNRNPCL1, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In one aspect, the compounds of Formula (I) or (II) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I) or (II) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I) or (II) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I) or (II) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I) or (II). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I) or (II) or a pharmaceutically acceptable salt, solvate, hydrate, tautomer, stereoisomer, or composition thereof.

In another aspect, the present disclosure features a method of altering the conformation of a nucleic acid (e.g., a DNA, RNA, e.g., a pre-mRNA) comprising contacting the nucleic acid with a compound of Formula (I) or (II) or a pharmaceutically acceptable salt, solvate, hydrate, tautomer, stereoisomer, or composition thereof. In an embodiment, the altering comprises forming a bulge or kink in the nucleic acid. In an embodiment, the altering comprises stabilizing a bulge or a kink in the nucleic acid. In an embodiment, the altering comprises reducing a bulge or a kink in the nucleic acid. In an embodiment, the nucleic acid comprises a splice site. In an embodiment, the compound of Formula (I) or (II) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I) or (II), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I) or (II), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The compounds of Formula (I) or (II) and pharmaceutically acceptable salts, solvates, hydrates, tautomers, stereoisomers, and compositions thereof, may induce apoptosis, and therefore, be useful in treating and/or preventing proliferative diseases.

In certain embodiments, the proliferative disease to be treated or prevented using the compounds of Formula (I) or (II) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AIL) (e.g., B-cell AIL, T-cell AIL), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the cancer is selected from adenoid cystic carcinoma (ACC), acute myelocytic leukemia (AML) (e.g., B-cell AML, T-cell AML), chronic myelocytic leukemia (CML) (e.g., B-cell CML, T-cell CML), non-Hodgkin lymphoma (NHL), Burkitt lymphoma, colorectal cancer (e.g., colon cancer, rectal cancer, colorectal adenocarcinoma), prostate cancer (e.g., prostate adenocarcinoma), ovarian cancer (e.g., cystadenocarcinoma, ovarian embryonal carcinoma, ovarian adenocarcinoma), and myelodysplastic syndrome (MD).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I) or (II) or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I) or (II), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I) or (II) or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I) or (II) or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I) or (II) or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I) or (II) or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I) or (II) or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I) or (II) or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I) or (II) or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I) or (II) increases expression of the haploinsufficient gene locus. In an embodiment, a compound of Formula (I) or (II) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I) or (II), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I) or (II) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I) or (II), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I) or (II) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I) or (II), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I) or (II) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In some embodiments, a compound of Formula (I), (II), or (III) or a pharmaceutically acceptable salt thereof, e.g., as described herein, has improved cell permeability over a reference compound, e.g., in a standard assay for measuring cell permeability. Cell permeability may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I), (II), or (III) or a pharmaceutically acceptable salt thereof, e.g., as described herein, has a cell permeability measurement (Papp) of <2×10⁻⁶ cm s⁻¹.

In an embodiment, a compound of Formula (I), (II), or (III) or a pharmaceutically acceptable salt thereof, e.g., as described herein, has a cell permeability measurement (Papp) of between 2-6×10⁻⁶ cm s⁻¹. In an embodiment, a compound of Formula (I), (II), or (III) or a pharmaceutically acceptable salt thereof, e.g., as described herein, has a cell permeability measurement (Papp) of Papp greater than 6×10⁻⁶ cm s⁻¹. In an embodiment, a compound of Formula (I), (II), or (III) or a pharmaceutically acceptable salt thereof, e.g., as described herein, has a cell permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), or (III) or a pharmaceutically acceptable salt thereof, e.g., as described herein, exhibits decreased cell efflux, e.g., over a reference compound, e.g., in a standard assay for measuring cell efflux. Cell efflux may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I), (II), or (III) or a pharmaceutically acceptable salt thereof, e.g., as described herein, has a cell efflux ratio of less than 1.5. In an embodiment, a compound of Formula (I), (II), or (III) or a pharmaceutically acceptable salt thereof, e.g., as described herein, has a cell efflux ratio of between 1.5 and 5. In an embodiment, a compound of Formula (I), (II), or (III) or a pharmaceutically acceptable salt thereof, e.g., as described herein, has a cell efflux ratio greater than 5. In an embodiment, a compound of Formula (I), (II), or (III) or a pharmaceutically acceptable salt thereof, e.g., as described herein, has a cell efflux ratio less than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), or (III) or a pharmaceutically acceptable salt thereof, e.g., as described herein, modulates the expression of a target protein (e.g., HTT or MYB) in a reference cell or sample. In an embodiment, a compound of Formula (I), (II), or (III) or a pharmaceutically acceptable salt thereof, e.g., as described herein, increases the expression of a target protein (e.g., HTT or MYB) in a reference cell or sample. In an embodiment, a compound of Formula (I), (II), or (III) or a pharmaceutically acceptable salt thereof, e.g., as described herein, decreases the expression of a target protein (e.g., HTT or MYB) in a reference cell or sample. The effect of an exemplary compound of Formula (I), (II), or (III) on protein abundance may be measured using a standard assay for measuring protein abundance, such as the HiBit-assay system (Promega). In this assay, percent response for each respective cell line may be as calculated at each compound concentration as follows: % response=100*(S−PC)/(NC−PC). For the normalized response at each concentration, a four-parameter logistical regression may be fit to the data and the response may be interpolated at the 50% value to determine a concentration for protein abundance at 50% (IC₅₀) an untreated control. In an embodiment, a compound of Formula (I), (II), or (III) or a pharmaceutically acceptable salt thereof, e.g., as described herein, has a protein abundance response less than 100 nM. In an embodiment, a compound of Formula (I), (II), or (III) or a pharmaceutically acceptable salt thereof, e.g., as described herein, has a protein abundance response between 100-1000 nM. In an embodiment, a compound of Formula (I), (II), or (III) or a pharmaceutically acceptable salt thereof, e.g., as described herein, has a protein abundance response greater than 1000 nM. In an embodiment, a compound of Formula (I), (II), or (III) or a pharmaceutically acceptable salt thereof, e.g., as described herein, has a protein abundance response greater than 10 uM. In an embodiment, a compound of Formula (I), (II), or (III) or a pharmaceutically acceptable salt thereof, e.g., as described herein, modulates the protein abundance of a target protein by about 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), or (III) or a pharmaceutically acceptable salt thereof, e.g., as described herein, modulates the viability of a target cell in a subject or sample. In an embodiment, a compound of Formula (I), (II), or (III) or a pharmaceutically acceptable salt thereof, e.g., as described herein, increases the viability of a target cell in a subject or sample. In an embodiment, a compound of Formula (I), (II), or (III) or a pharmaceutically acceptable salt thereof, e.g., as described herein, decreases the viability of a target cell in a subject or sample. In an embodiment, a compound of Formula (I), (II), or (III) or a pharmaceutically acceptable salt thereof, e.g., as described herein, does not impact the viability of a cell (e.g., is non-toxic) in a subject or sample. The effect an exemplary compound of Formula (I), (II), or (III) on cell viability may be measured using a standard assay for measuring cell toxicity, such as the Cell Titer Glo 2.0 assay in either K562 (human chronic myelogenous leukemia) or SH-SY5Y (human neuroblastoma) cells. The concentration at which cell viability is measured may be based on the particular assay used. In an embodiment, a compound of Formula (I), (II), or (III) or a pharmaceutically acceptable salt thereof, e.g., as described herein, is tolerated by a target cell at a concentration of less than 100 nM. In an embodiment, a compound of Formula (I), (II), or (III) or a pharmaceutically acceptable salt thereof, e.g., as described herein, is tolerated by a target cell at a concentration of between 100-1000 nM. In an embodiment, a compound of Formula (I), (II), or (III) or a pharmaceutically acceptable salt thereof, e.g., as described herein, is tolerated by a target cell at a concentration of greater than 1000 nM. In an embodiment, a compound of Formula (I), (II), or (III) or a pharmaceutically acceptable salt thereof, e.g., as described herein, is tolerated by a target cell at a concentration of greater than 10 uM.

In some embodiments, a compound of Formula (I), (II), or (III) or a pharmaceutically acceptable salt thereof, e.g., as described herein, has improved brain permeability over a reference compound, e.g., in a standard assay for measuring brain permeability. Brain permeability may be measured, for example, by determining the unbound partition coefficient (Kpuu), brain. In such an assay, the unbound brain partition coefficient (K_{p,uu,bmin}) may be defined as the ratio of unbound brain-free compound concentration to unbound plasma concentration. It is calculated using the following equation:

$${K}_{p , uu , brain} = \frac{{f}_{u , brain} \times {C}_{brain}}{{f}_{u , plasma} \times {C}_{plasma}}$$

C_{brain} and Cₚₗₐₛₘₐ represent the total concentrations in brain and plasma, respectively. In this assay, the f_{u,brain} and f_{u,plasma} may be the unbound fraction of the compound in brain and plasma, respectively. Both f_{u,brain} and f_{u,plasma} may be determined in vitro via equilibrium dialysis. In an embodiment, a compound of Formula (I), (II), or (III) or a pharmaceutically acceptable salt thereof, e.g., as described herein, has a Kp value of greater than 5. In an embodiment, a compound of Formula (I), (II), or (III) or a pharmaceutically acceptable salt thereof, e.g., as described herein, has a Kp value between 1 and 5. In an embodiment, a compound of Formula (I), (II), or (III) or a pharmaceutically acceptable salt thereof, e.g., as described herein, has a Kp value between 0.2-1. In an embodiment, a compound of Formula (I), (II), or (III) or a pharmaceutically acceptable salt thereof, e.g., as described herein, has a Kp value of less than 0.2. In an embodiment, a compound of Formula (I), (II), or (III) or a pharmaceutically acceptable salt thereof, e.g., as described herein, has a Kpuu value of greater than 2.5. In an embodiment, a compound of Formula (I), (II), or (III) or a pharmaceutically acceptable salt thereof, e.g., as described herein, has a Kpuu value between 0.5-2.5. In an embodiment, a compound of Formula (I), (II), or (III) or a pharmaceutically acceptable salt thereof, e.g., as described herein, has a Kpuu value between 0.1-0.5. In an embodiment, a compound of Formula (I), (II), or (III) or a pharmaceutically acceptable salt thereof, e.g., as described herein, has a Kpuu value of less than 0.1. In an embodiment, a compound of Formula (I), (II), or (III) or a pharmaceutically acceptable salt thereof, e.g., as described herein, has a brain permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), or (III) or a pharmaceutically acceptable salt thereof, e.g., as described herein, exhibits selectivity for one target nucleic acid sequence, e.g., pre-mRNA transcript sequence or bulge, compared to another target nucleic acid sequence, e.g., pre-mRNA transcript sequence or bulge. In an embodiment, a compound of Formula (I), (II), or (III) or a pharmaceutically acceptable salt thereof, e.g., as described herein, exhibits selectivity for HTT, e.g., an HTT-related nucleic acid sequence. In an embodiment, a compound of Formula (I), (II), or (III) or a pharmaceutically acceptable salt thereof, e.g., as described herein, exhibits selectivity for SMN2, e.g., an SMN2-related nucleic acid sequence. In an embodiment, a compound of Formula (I), (II), or (III) or a pharmaceutically acceptable salt thereof, e.g., as described herein, exhibits selectivity for Target C, e.g., a Target C-related nucleic acid sequence. In an embodiment, a compound of Formula (I), (II), or (III) or a pharmaceutically acceptable salt thereof, e.g., as described herein, exhibits selectivity for MYB, e.g., a MYB-related nucleic acid sequence. Selectivity for one target nucleic acid sequence over another may be measured using any number of methods known in the art. In an embodiment, selectivity may be measured by determining the ratio of derived qPCR values (e.g., as described herein) for one target nucleic acid sequence over another. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for one target nucleic acid sequence over another. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over another target nucleic acid sequence. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over another. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over another target nucleic acid sequence. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for Target C sequence over another. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over MYB. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over HTT. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over SMN2. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over HTT. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over MYB. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over SMN2. In an embodiment, a compound of Formula (I), (II), or (III) or a pharmaceutically acceptable salt thereof, e.g., as described herein, has a 3-fold greater selectivity for HTT over MYB. In an embodiment, a compound of Formula (I), (II), or (III) or a pharmaceutically acceptable salt thereof, e.g., as described herein, has a 3-fold greater selectivity for MYB over HTT. In an embodiment, a compound of Formula (I), (II), or (III) or a pharmaceutically acceptable salt thereof, e.g., as described herein, has a 10-fold greater selectivity for HTT over MYB. In an embodiment, a compound of Formula (I), (II), or (III) or a pharmaceutically acceptable salt thereof, e.g., as described herein, has a 10-fold greater selectivity for MYB over HTT. In an embodiment, a compound of Formula (I), (II), or (III) or a pharmaceutically acceptable salt thereof, e.g., as described herein, has a 3-fold greater selectivity for HTT over SMN2. In an embodiment, a compound of Formula (I), (II), or (III) or a pharmaceutically acceptable salt thereof, e.g., as described herein, has a 3-fold greater selectivity for SMN2 over HTT. In an embodiment, a compound of Formula (I), (II), or (III) or a pharmaceutically acceptable salt thereof, e.g., as described herein, has a 10-fold greater selectivity for HTT over SMN2. In an embodiment, a compound of Formula (I), (II), or (III) or a pharmaceutically acceptable salt thereof, e.g., as described herein, has a 10-fold greater selectivity for SMN2 over HTT. In an embodiment, a compound of Formula (I), (II), or (III) or a pharmaceutically acceptable salt thereof, e.g., as described herein, has a 3-fold greater selectivity for MYB over SMN2. In an embodiment, a compound of Formula (I), (II), or (III) or a pharmaceutically acceptable salt thereof, e.g., as described herein, has a 3-fold greater selectivity for SMN2 over MYB. In an embodiment, a compound of Formula (I), (II), or (III) or a pharmaceutically acceptable salt thereof, e.g., as described herein, has a 10-fold greater selectivity for MYB over SMN2. In an embodiment, a compound of Formula (I), (II), or (III) or a pharmaceutically acceptable salt thereof, e.g., as described herein, has a 10-fold greater selectivity for SMN2 over MYB. In an embodiment, a compound of Formula (I), (II), or (III) or a pharmaceutically acceptable salt thereof, e.g., as described herein, has a selectivity for one target nucleic acid sequence that is greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a second nucleic acid sequence.

In certain embodiments, the methods described herein comprise the additional step of administering one or more additional pharmaceutical agents in combination with the compound of Formula (I) or (II), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject. Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (6) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 m, 120 Å, 40° C.) operating in ESI(+) ionization mode; flow rate=1.2 mL/min. Mobile phase=0.05% TFA in water or CH₃CN; or on Shimadzu-2020EV using column: Poroshell HPH—C18 (C18, Ø4.6×50 mm, 3 m, 120 Å, 40° C.) operating in ESI(+) ionization mode; flow rate=1.2 mL/min. Mobile phase A: Water/5 mM NH₄HCO₃, Mobile phase B: CH₃CN.)

Analytical chiral HPLC: Analytical chiral HPLC was performed on a Agilent 1260 using column: CHIRRALPAK IG-3, CHIRALPAK IC-3 or CHIRALPAK OJ-3, with flow rate=1.2 mL/min. Mobile phase=MTBE(DEA):EtOH=50:50). Preparative HPLC purification: prep-HPLC purification was performed using one of the following HPLC conditions: Condition 1: BioBasic C-18 column (length: 250, diameter: 21.2 mm, particle size: 5 μm); Flow rate=5 mL/min); Mobile Phase A: water (containing 0.1% TFA); Mobile Phase B: acetonitrile; Gradient of 10-90% B over 90 min.

Preparative chiral HPLC: purification by chiral HPLC was performed on a Gilson-GX 281 using column: CHERALPAK IG-3, CHIRALPAK IC-3 or CHIRALPAK OJ-3.

### General Synthetic Schemes

Compounds of the present disclosure may be prepared using one of the synthetic protocols illustrated below in Schemes A-C.

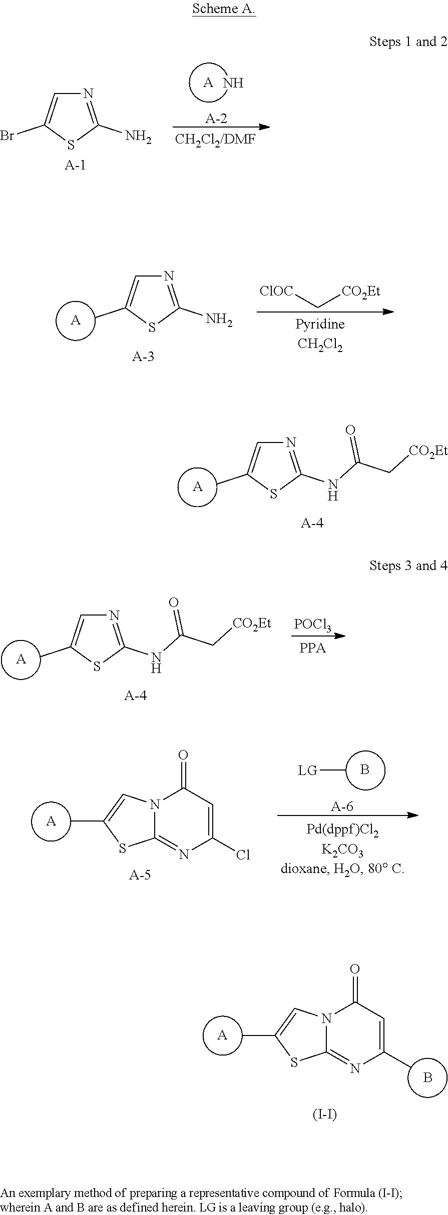

An exemplary method of preparing a compound of Formula (I-I) is provided in Scheme A. In this scheme, A-3 is prepared in Step 1 by incubating A-1 with A-2 in a mixture of dichloromethane and dimethylformamide, or a similar mixture of solvents. In Step 2, A-4 is prepared by treating A-3 with ethyl malonyl chloride and pyridine, or similar reagents, in dichloromethane or any other suitable solvent.

A-4 is then cyclized in Step 3, by treating A-4 with phosphoryl chloride and polyphosphoric acid (PPA), or other reagents sufficient to affect the conversion of A-4 to of A-5. Next, A-5 and A-6 are coupled to provide a compound of Formula (I-I) in Step 4. This coupling reaction may be conducted in the presence of Pd(dppf)Cl₂, and K₂CO₃ or a similar reagent. Alternative catalysts to Pd(dppf)Cl₂ may be used, such as any suitable palladium catalyst. The reaction of Step 4 is conducted in a mixture of dioxane and water, or other suitable solvent mixtures, and the reaction is heated to 80° C. or a temperature sufficient to provide the compound of Formula (I-I). Each starting material and/or intermediate in Scheme A may be protected and deprotected using standard protecting group methods. In addition, purification and characterization of each intermediate as well as the final compound of Formula (I) may be afforded by any accepted procedure.

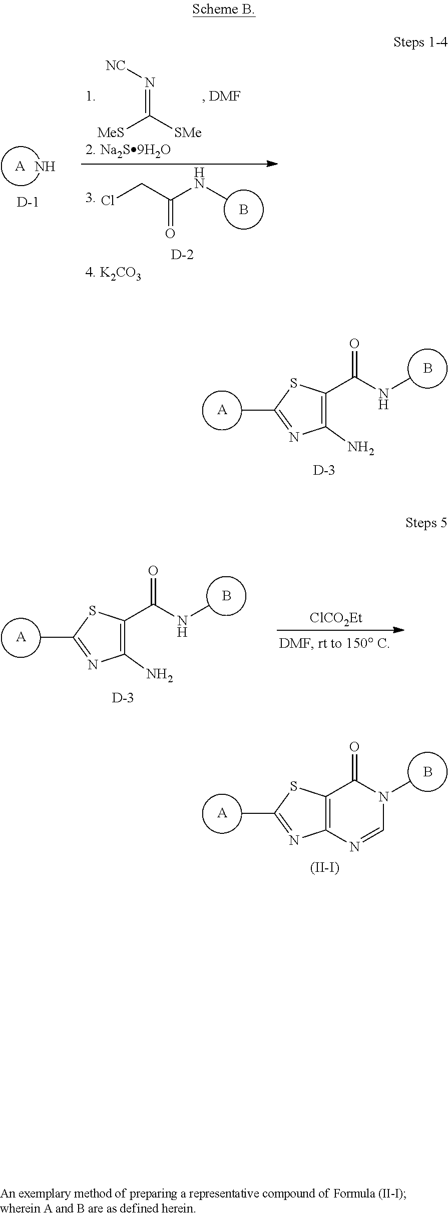

An exemplary method of preparing a compound of Formula (II-I) is provided in Scheme B. In this scheme, D-3 is first prepared using a multistep protocol, which involves incubating D-1 with dimethyl N-cyanodithioiminocarbonate or a similar reagent, in a suitable solvent such as dimethylformamide (DMF). The intermediate formed by this step is then treated with sodium sulfide nonahydrate, or a suitable alternative, followed by incubation with D-2. The resulting mixture is then treated with potassium carbonate, or a similar reagent, to afford D-3. In Step 5, D-3 is converted to a compound of Formula (II-I) through a cyclization reaction, which involves treating D-3 with ethyl chloroformate or a suitable alternative, in DMF or a similar solvent. The reaction of Step 5 may be carried out initially at room temperature with heating to 150° C., or at a temperature sufficient to provide the compound of Formula (II-I). Each starting material and/or intermediate in Scheme B may be protected and deprotected using standard protecting group methods. In addition, purification and characterization of each intermediate as well as the final compound of Formula (II) may be afforded by any accepted procedure.

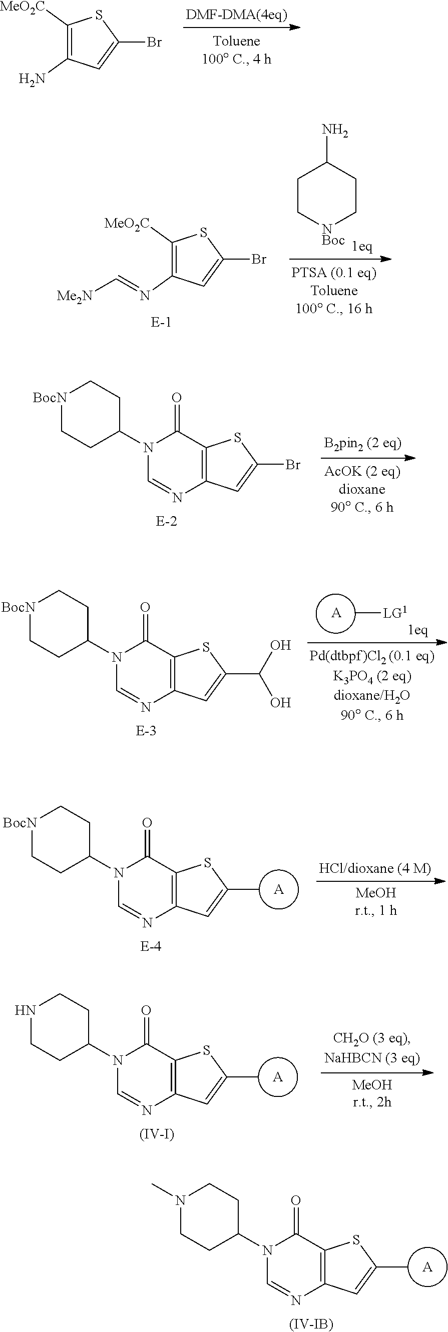

### Example 1: Synthesis of Compound 182

### Synthesis of Intermediate B18

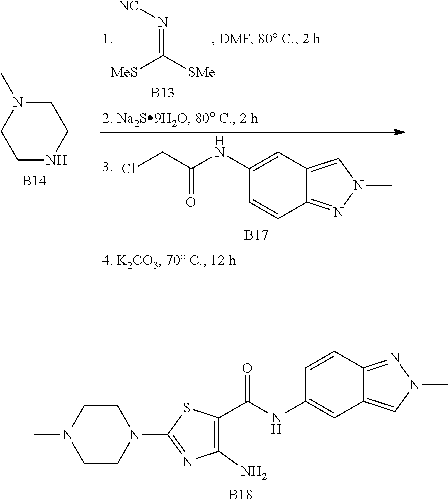

N-Cyanodithioiminocarbonate (B13; 44 mg, 0.3 mmol) was added to a solution of N-methyl piperazine (B14; 30 mg, 0.3 mmol) in dimethylformamide (0.5 mL), and the reaction mixture was heated to 80° C. for 2 h. Sodium sulfide nonahydrate (72 mg, 0.3 mmol) was then added, and the reaction mixture was stirred at 80° C. for 2 h. A solution of 2-chloro-N-(2-methyl-2H-indazol-5-yl)acetamide (B17; 134 mg, 0.6 mmol) in dimethylformamide (1 mL) was then added dropwise, and the reaction mixture was stirred at 70° C. for 2 h. Next, K₂CO₃ (42 mg, 0.3 mmol) was added and the reaction mixture was stirred at 70° C. for an additional 12 h. The volatiles were then removed under reduced pressure, and the crude material was purified by reverse phase chromatography on a C18 column, eluting with acetonitrile in neutral water (0-100%), to afford 4-amino-N-(2-methyl-2H-indazol-5-yl)-2-(4-methylpiperazin-1-yl)thiazole-5-carboxamide (B18; 44 mg) as a solid. LCMS (ES, m/z): 372.1 [M+H]⁺.

### Synthesis of Compound 182

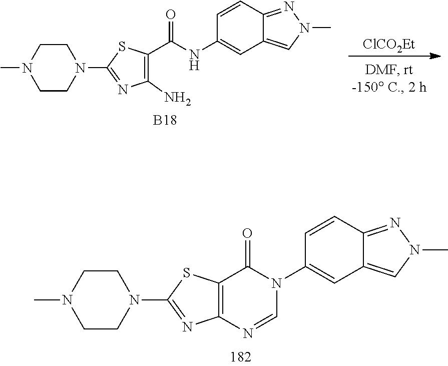

Ethyl chloroformate (0.15 mL, 1.54 mmol) was added to a solution of 4-amino-N-(2-methyl-2H-indazol-5-yl)-2-(4-methylpiperazin-1-yl)thiazole-5-carboxamide (B18; 44 mg, 0.12 mmol) in dimethylformamide (2 mL), and the reaction mixture was stirred at room temperature for 10 min, and then at 150° C. for an additional 2 h. The volatiles were then removed under reduced pressure, and a saturated solution of NaHCO₃ (10 mL) and dichloromethane (15 mL) were added and the layers were separated. The aqueous phase was further extracted with dichloromethane (3×15 mL), and the combined organic layers were dried over Na₂SO₄, filtered and concentrated under reduced pressure. The residue was purified by silica gel column chromatography eluting with 0-10% methanol in dichloromethane, to afford 6-(2-methyl-2H-indazol-5-yl)-2-(4-methylpiperazin-1-yl)thiazolo[4,5-d]pyrimidin-7(6H)-one (Compound 182; 20 mg) as a solid. LCMS (ES, m/z): 382.1 [M+H]⁺. ¹H NMR (DMSO-d₆, 400 MHz): δ_{H} 8.47 (1H, s), 8.40 (1H, s), 7.83 (1H, s), 7.67 (1H, d, J=9.1 Hz), 7.25 (1H, dd, J=9.1, 2.0 Hz), 4.20 (3H, s), 3.62 (4H, m), 2.44 (4H, m), 2.23 (3H, s).

### Example 2: Synthesis of Compound 183

### Synthesis of Intermediate B22

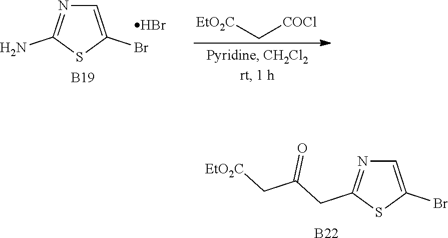

Pyridine (1.3 mL, 16.1 mmol) was added to a suspension of 2-amino-5-bromothiazole hydrobromide (B19; 1.06 g, 4.06 mmol) in dichloromethane (2.5 mL), and the resulting solution was then added dropwise to a solution of ethyl malonylchloride (0.7 mL, 5.47 mmol) in dichloromethane (4 mL), and the resulting mixture was stirred at room temperature for 1 h. The reaction mixture was then poured into water (36 mL), and an excess of sodium carbonate was carefully added with stirring, and the mixture was stirred at room temperature for 1 h. The mixture was then diluted with dichloromethane (20 mL), the organic layer was then collected, and the aqueous phase was further extracted with dichloromethane (2×20 mL). The combined organic extracts were washed with water (30 mL), dried over Na₂SO₄, filtered and concentrated in vacuo to provide an oil which was triturated in diethyl ether (10 mL). The resulting solids were collected by vacuum filtration to afford ethyl 3-((5-bromothiazol-2-yl)amino)-3-oxopropanoate (B22; 335 mg). LCMS (ES, m/z): 292.9, 294.9 [M+H]⁺.

### Synthesis of Intermediate B23

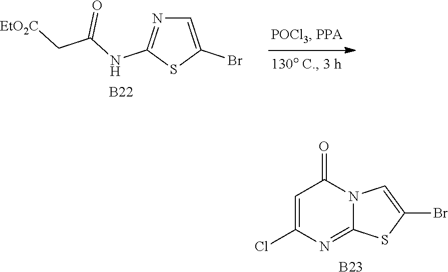

Polyphosphoric acid (111 mg, 0.46 mmol) was added to ethyl 3-((5-bromothiazol-2-yl)amino)-3-oxopropanoate (B22; 335 mg, 1.14 mmol), followed by the addition of phosphorous oxychloride (0.32 mL, 3.43 mmol), and the resulting mixture was stirred at 125° C. for 3 h. After cooling the mixture, anhydrous ethanol (1.1 mL) was added and the mixture was sonicated until homogeneous, and then heated to reflux while stirring for 30 min. The mixture was then cooled to room temperature, diluted with dichloromethane (20 mL), and aqueous NaHCO₃ was added until the mixture was basic. The layers were then separated and the aqueous phase was further extracted with dichloromethane (2×15 mL). The combined organic layers were then dried over Na₂SO₄ and concentrated. The residue was dissolved in a minimum amount of dichloromethane and purified by silica gel column chromatography eluting with ethyl acetate in dichloromethane (0 to 10%), to afford 2-bromo-7-chloro-5H-thiazolo[3,2-a]pyrimidin-5-one (B23) as a solid.

LCMS (ES, m/z): 264.8, 266.8 [M+H]⁺.

### Synthesis of Intermediate B24

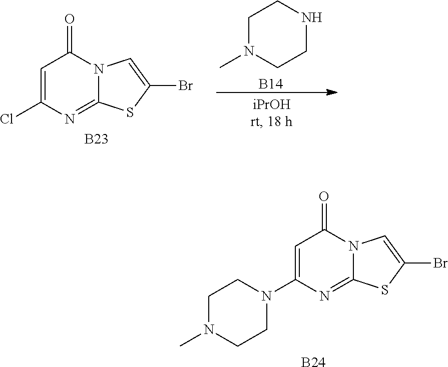

N-Methyl piperazine (B14; 0.1 mL, 0.9 mmol) was added to a suspension of 2-bromo-7-chloro-5H-thiazolo[3,2-a]pyrimidin-5-one (B23; 140 mg, 0.53 mmol) in isopropyl alcohol (2 mL), and the resulting mixture was stirred at room temperature for 18 h. The solvent was then removed in vacuo and the remaining solid was triturated in diethyl ether (3 mL), and further purified by chromatography on silica gel eluting with 0 to 30% methanol in dichloromethane, to afford 2-bromo-7-(4-methylpiperazin-1-yl)-5H-thiazolo[3,2-a]pyrimidin-5-one (B24) as a solid. LCMS (ES, m/z): 329.0, 331.0 [M+H]⁺.

### Synthesis of Compound 183

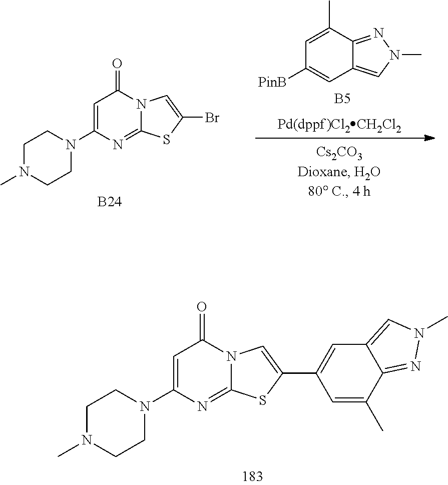

A mixture of 2-bromo-7-(4-methylpiperazin-1-yl)-5H-thiazolo[3,2-a]pyrimidin-5-one (B24; 52.5 mg, 0.16 mmol), 2,7-dimethyl-5-(4,4,5,5-tetramethyl-1,3,2-dioxaborolan-2-yl)-2H-indazole (B5; 63 mg, 0.23 mmol), Pd(dppf)Cl₂—CH₂Cl₂ (11 mg, 0.015 mmol) and cesium carbonate (163 mg, 0.50 mmol) was suspended in dioxane (0.5 mL) and water (0.05 mL), and the resulting mixture was stirred at 80° C. for 4 h. The reaction mixture was then diluted with dichloromethane (3 mL), filtered through Celite, and concentrated. The residue was purified by chromatography on silica gel eluting with 10 to 40% of methanol in dichloromethane to provide a solid (37 mg), which was triturated in a mixture of ethanol (0.7 mL) and diethyl ether (3 mL), and the resulting solid was collected by vacuum filtration to afford 2-(2,7-dimethyl-2H-indazol-5-yl)-7-(4-methylpiperazin-1-yl)-5H-thiazolo[3,2-a]pyrimidin-5-one (Compound 183; 23.7 mg) as a solid. LCMS (ES, m/z): 395.2 [M+H]⁺. ¹H NMR (CDCl₃, 400 MHz): δ_{H} 8.02 (1H, s), 7.93 (1H, s), 7.55 (1H, s), 7.23 (1H, s), 5.40 (1H, s), 4.25 (3H, s), 3.64 (4H, br s), 2.65 (3H, s), 2.48 (4H, br s), 2.34 (3H, s).

### Example 3: Synthesis of Compound 184

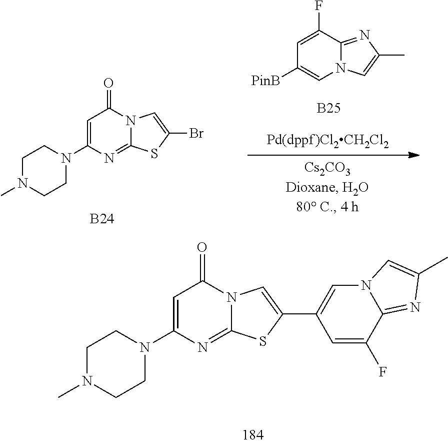

A mixture of 2-bromo-7-(4-methylpiperazin-1-yl)-5H-thiazolo[3,2-a]pyrimidin-5-one (B24 from Example 5; 51 mg, 0.16 mmol), 8-fluoro-2-methyl-6-(4,4,5,5-tetramethyl-1,3,2-dioxaborolan-2-yl)imidazo[1,2-a]pyridine (B25; 52 mg, 0.19 mmol), Pd(dppf)Cl₂—CH₂Cl₂ (13 mg, 0.018 mmol) and cesium carbonate (135 mg, 0.41 mmol) was suspended in dioxane (0.58 mL) and water (0.08 mL), and stirred at 80° C. for 4.5 h. The reaction mixture was then diluted with dichloromethane (20 mL), filtered through Celite, and concentrated. The residue was purified by column chromatography on silica gel eluting with 20 to 50% methanol in dichloromethane. Selected fractions obtained from the column were concentrated to a volume of 0.5 mL, resulting in a suspension which was treated with diethyl ether (3 mL), triturated, and the solids were collected by vacuum filtration, to afford 2-(2,7-dimethyl-2H-indazol-5-yl)-7-(4-methylpiperazin-1-yl)-5H-thiazolo[3,2-a]pyrimidin-5-one (Compound 184; 34 mg) as a solid. LCMS (ES, m/z): 399.1 [M+H]⁺. 1H NMR (CDCl₃:DMSO-d₆ (9:1), 400 MHz): δ_{H} 7.99 (2H, d, J=7.1 Hz), 7.46 (1H, s), 7.01 (1H, d, J=10.6 HZ), 5.38 (1H, s), 4.25 (3H, s), 3.64 (4H, br s), 2.49 (4H, br s), 2.34 (3H, s).

### Example 4: Synthesis of Compound 225

### Synthesis of Intermediate B45

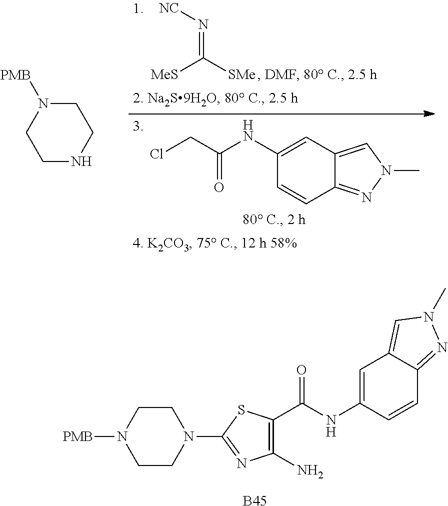

To a solution of 1-(4-methoxybenzyl)piperazine (0.10 g, 0.48 mmol) in DMF (1.5 mL) was added dimethyl-N-cyanodithioiminocarbonate (0.071 g, 0.48 mmol). The reaction mixture was heated to 80° C. for 2.5 h. Na₂S·9H₂O (0.12 g, 0.48 mmol) was then added and the reaction mixture was stirred at 80° C. for an additional 2.5 h. A solution of 2-chloro-N-(2-methyl-2H-indazol-5-yl)acetamide (0.32 g, 1.45 mmol) in DMF (1.5 mL) was added dropwise, and the reaction mixture was stirred at 80° C. for an additional 2 h. K₂CO₃ (0.067 g, 0.48 mmol) was added and the reaction mixture was stirred at 75° C. for 12 h. The reaction mixture was filtered and concentrated in vacuo to give a residue. The residue was purified by reverse phase chromatography (C18) using 0-100% acetonitrile in water to afford 4-amino-2-(4-(4-methoxybenzyl)piperazin-1-yl)-N-(2-methyl-2H-indazol-5-yl)thiazole-5-carboxamide (135 mg, 58%) as a solid. LCMS (ES, m/z): 478.0 [M+H]⁺.

### Synthesis of Intermediate B46

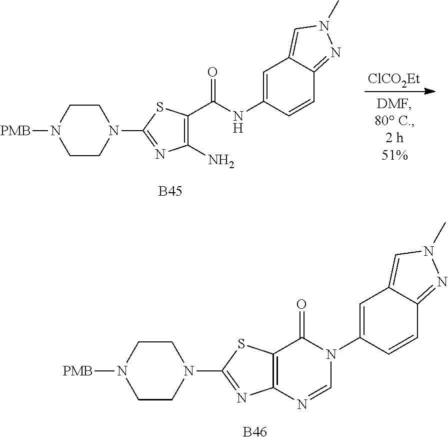

To a solution of 4-amino-2-(4-(4-methoxybenzyl)piperazin-1-yl)-N-(2-methyl-2H-indazol-5-yl)thiazole-5-carboxamide (0.13 g, 0.28 mmol) in DMF (7.0 mL) was added ethyl chloroformate (0.38 mL, 4.0 mmol). The reaction mixture was stirred at room temperature for 10 min, then at 80° C. for 2 h. The reaction mixture was concentrated in vacuo to a residue and partitioned between a saturated solution of NaHCO₃ (15 mL) and DCM (20 mL). The layers were separated, and the aqueous layer was extracted with DCM (3×20 mL). The organic layers were combined, dried over Na₂SO₄, filtered, and concentrated under reduced pressure. The crude product was purified by column chromatography on silica gel using a gradient of 0-10% MeOH in DCM to afford 2-(4-(4-methoxybenzyl)piperazin-1-yl)-6-(2-methyl-2H-indazol-5-yl)thiazolo[4,5-d]pyrimidin-7(6H)-one (0.070 g, 51%) as a solid. LCMS (ES, m/z): 488.1 [M+H]⁺.

### Synthesis of Compound 225

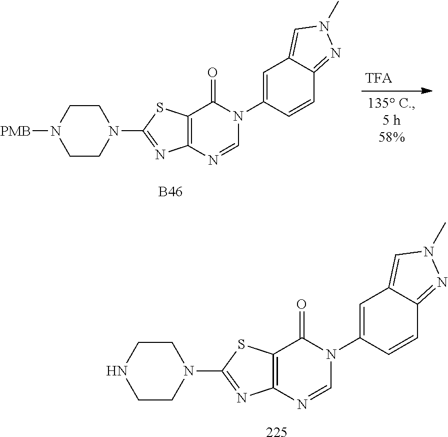

A solution of 2-(4-(4-methoxybenzyl)piperazin-1-yl)-6-(2-methyl-2H-indazol-5-yl)thiazolo[4,5-d]pyrimidin-7(6H)-one (30.0 mg, 0.062 mmol) in TFA (2.0 mL) was heated to 135° C. for 5 h.

The reaction mixture was diluted with DCM (10 mL) and basified with a saturated solution of NaHCO₃ until pH ˜8. The layers were separated, and the aqueous phase was extracted with DCM (3×10 mL). The organic layers were combined, dried over Na₂SO₄, filtered, and concentrated in vacuo to give a residue. The residue was purified by column chromatography on silica gel using a gradient of 0-20% MeOH in DCM to afford 6-(2-methyl-2H-indazol-5-yl)-2-(piperazin-1-yl)thiazolo[4,5-d]pyrimidin-7(6H)-one (13 mg, 58%) as a solid. LCMS (ES, m/z): 368.1 [M+H]⁺. 1H NMR (DMSO-d₆, 400 MHz): δ_{H} 8.48 (1H, s), 8.40 (1H, s), 7.84 (1H, s), 7.69 (1H, d, J=9.1 Hz), 7.26 (1H, d, J=9.2 Hz), 4.21 (3H, s), 3.56 (4H, s), 2.83 (4H, s).

### Example 5: Synthesis of Compound 226

### Synthesis of Intermediate B47

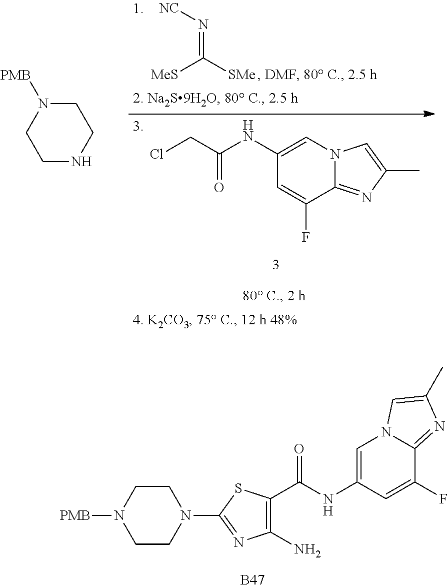

To a solution of 1-(4-methoxybenzyl)piperazine (0.10 g, 0.48 mmol) in DMF (1.5 mL) was added dimethyl-N-cyanodithioiminocarbonate (0.071 g, 0.48 mmol). The reaction mixture was heated to 80° C. for 2.5 h. Na₂S·9H₂O (0.11 g, 0.48 mmol) was added and the reaction mixture was stirred at 80° C. for an additional 2.5 h. A solution of 2-chloro-N-(8-fluoro-2-methylimidazo[1,2-a]pyridin-6-yl)acetamide (0.32 g, 1.4 mmol) in DMF (1.5 mL) was added dropwise, and the reaction mixture was stirred at 80° C. for an additional 2 h. K₂CO₃ (0.067 g, 0.48 mmol) was added, and the reaction mixture was stirred at 75° C. for 12 h. The reaction mixture was concentrated in vacuo to a residue. The residue was purified by reverse phase chromatography (C18) using 0-100% acetonitrile in water to afford 4-amino-N-(8-fluoro-2-methylimidazo[1,2-a]pyridin-6-yl)-2-(4-(4-methoxybenzyl)piperazin-1-yl)thiazole-5-carboxamide (0.11 g, 48%) as a solid. LCMS (ES, m/z): 495.9 [M+H]⁺.

### Synthesis of Intermediate B48

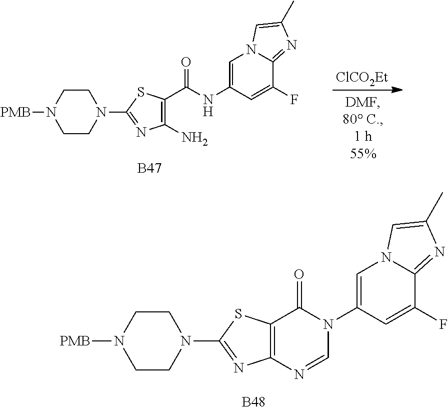

To a solution of 4-amino-N-(8-fluoro-2-methylimidazo[1,2-a]pyridin-6-yl)-2-(4-(4-methoxybenzyl)piperazin-1-yl)thiazole-5-carboxamide (0.11 g, 0.23 mmol) in DMF (6.0 mL) was added ethyl chloroformate (0.30 mL, 3.2 mmol). The reaction mixture was stirred at room temperature for 10 min, then at 80° C. for 1 h. The reaction mixture was concentrated in vacuo to give a residue. The residue was partitioned between a saturated solution of NaHCO₃ (10 mL) and DCM (15 mL), and the layers were separated. The aqueous layer was extracted with DCM (3×15 mL). The organic layers were combined, dried over Na₂SO₄, filtered, and concentrated in vacuo to give a residue. The residue was purified by column chromatography on silica gel using a gradient of 0-10% MeOH in ethyl acetate to afford 6-(8-fluoro-2-methylimidazo[1,2-a]pyridin-6-yl)-2-(4-(4-methoxybenzyl)piperazin-1-yl)thiazolo[4,5-d]pyrimidin-7(6H)-one (0.064 g, 55%) as a solid. LCMS (ES, m/z): 505.9 [M+H]⁺.

### Synthesis of Compound 226

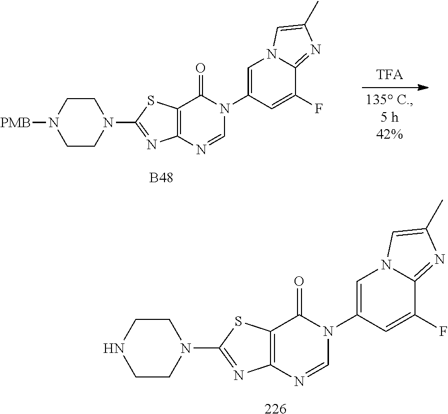

A solution of 6-(8-fluoro-2-methylimidazo[1,2-a]pyridin-6-yl)-2-(4-(4-methoxybenzyl)piperazin-1-yl)thiazolo[4,5-d]pyrimidin-7(6H)-one (62.0 mg, 0.12 mmol) in TFA (6.0 mL) was heated to 135° C. for 5 h. The reaction mixture was diluted with DCM (20 mL), basified with a saturated solution of NaHCO₃ and the aqueous and organic layers were separated. The aqueous phase was extracted with DCM (3×20 mL). The organic layers were combined, dried over Na₂SO₄, filtered and concentrated in vacuo to give a residue. The residue was purified by column chromatography on silica gel using a gradient of 0-20% MeOH in DCM to afford 6-(8-fluoro-2-methylimidazo[1,2-a]pyridin-6-yl)-2-(piperazin-1-yl)thiazolo[4,5-d]pyrimidin-7(6H)-one (20 mg, 42%) as a solid. LCMS (ES, m/z): 386.1 [M+H]⁺. ¹H NMR (DMSO-d₆, 400 MHz): δ_{H} 8.75 (1H, s), 8.46 (1H, s), 7.91 (1H, s), 7.41 (1H, d, J=11.5 Hz), 3.55 (4H, s), 2.82 (4H, s), 2.39 (3H, s).

### Example 6: Synthesis of Compound 109

### Synthesis of Intermediate B49

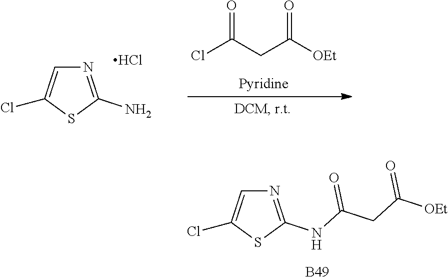

Pyridine (9.8 mL, 121 mmol) was added to 5-chlorothiazol-2-amine hydrochloride (5.25 g, 30.7 mmol) in DCM (15 mL). The solution was added dropwise to a solution of ethyl malonyl chloride (5.5 mL, 43.0 mmol) in DCM (15 mL) chilled in an ice bath. The resulting mixture was warmed to room temperature and stirred for 2 hours. The reaction mixture was filtered through celite, and the filter cake washed with DCM. The filtrate was concentrated under reduced pressure and the residue was purified by flash chromatography on silica gel using a gradient of 0-20% MeOH in DCM. Fractions containing product were combined and the solvent was evaporated in vacuo to give a residue. The residue was triturated with TBME (30 mL) and filtered to afford ethyl 3-((5-chlorothiazol-2-yl)amino)-3-oxopropanoate (6.0 g, 79%) as a solid.

LCMS (ES, m/z): 249.0 [M+H]⁺.

### Synthesis of Intermediate B50

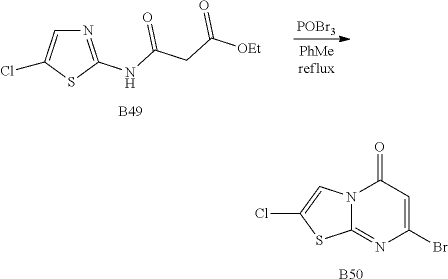 [SMILES: "B49" ; CCOC(=O)CC(=O)Nc1ncc(Cl)s1 ; "B50" ; O=c1cc(Br)nc2sc(Cl)cn12 ; "POBr3"]

To phosphorus(V) oxybromide (3.50 g, 12.2 mmol) was added ethyl 3-((5-chlorothiazol-2-yl)amino-3-oxopropanoate (600 mg, 2.41 mmol) and dissolved in 10 ml of toluene under argon atmosphere while stirring. The reaction mixture was refluxed overnight, then cooled to room temperature, and the solution was poured out leaving a thick oil in the reaction vessel. The oil was dissolved in DMF (6 mL), and stirred for 20 minutes. The resulting solution was combined with the reaction mixture and diluted with ethyl acetate (50 mL), washed with saturated NaHCO₃ (3×25 mL), brine (2×25 mL), dried over anhydrous Na₂SO₄, filtered, and concentrated in vacuo to give a residue. The residue was purified by flash chromatography on a silica gel column using a gradient of 10-100% EtOAc in hexanes. Selected fractions were combined and concentrated in vacuo to afford 7-bromo-2-chloro-5H-thiazolo[3,2-a]pyrimidin-5-one (385 mg, 36%) as a solid. LCMS (ES, m/z): 264.9 [M+H]⁺.

### Synthesis of Intermediate B51

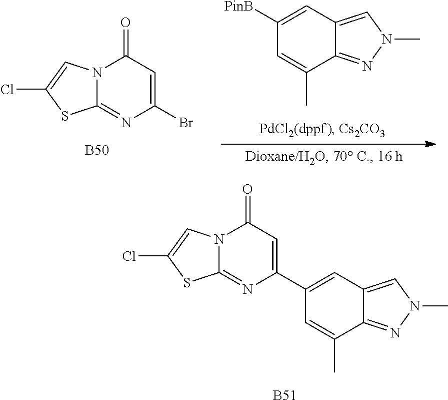

7-bromo-2-chloro-5H-thiazolo[3,2-a]pyrimidin-5-one (13 mg, 0.049 mmol), 2,7-dimethyl-5-(4,4,5,5-tetramethyl-1,3,2-dioxaborolan-2-yl)-2H-indazole (14 mg, 0.040 mmol), PdCl₂(dppf) (4.0 mg, 0.005 mmol) and Cs₂CO₃ (32 mg, 0.098 mmol) were dissolved in dioxane (0.7 mL) and H₂O (0.1 mL) in a sealed tube, and heated at 70° C. for 16 h under argon atmosphere. The reaction mixture was diluted with ethyl acetate (10 mL), then washed with saturated NaHCO₃ (10 mL) and brine (10 mL). The organic phase was dried over Na₂SO₄, filtered, and concentrated in vacuo to afford 2-chloro-7-(2,7-dimethyl-2H-indazol-5-yl)-5H-thiazolo[3,2-a]pyrimidin-5-one (31 mg) as a solid. LCMS (ES, m/z): 331.0 [M+H]⁺.

### Synthesis of Compound 109

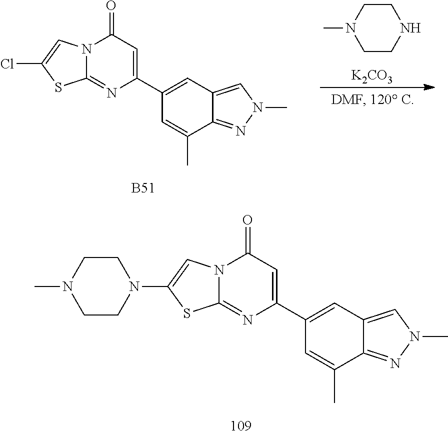

2-chloro-7-(2,7-dimethyl-2H-indazol-5-yl)-5H-thiazolo[3,2-a]pyrimidin-5-one (31 mg, 0.094 mmol), K₂CO₃ (40 mg, 0.289 mmol), and N-methylpiperazine 7 (29 mg, 0.290 mmol) were dissolved in DMF (1 mL) and stirred at 120° C. for 5 hours. The reaction mixture was cooled to room temperature, diluted with ethyl acetate (25 mL), washed with brine (2×20 mL), dried over anhydrous Na₂SO₄, filtered, and concentrated in vacuo to give a residue. The residue was purified by flash chromatography on a C18 column using a gradient of 5-70% MeCN in water with 0.1% formic acid additive. Fractions containing product were combined, neutralized with (NH₄)₂CO₃, and lyophilized to afford 7-(2,7-dimethyl-2H-indazol-5-yl)-2-(4-methylpiperazin-1-yl)-5H-thiazolo[3,2-a]pyrimidin-5-one (8.7 mg, 24%) as a solid. LCMS (ES, m/z): 395.2 [M+H]⁺. 1H NMR (CDCl₃, 300 MHz): δ 8.22 (1H, s), 8.00 (1H, s), 7.64 (1H, s), 7.15 (1H, s), 6.75 (1H, s), 4.28 (3H, s), 3.34 (4H, m), 2.78 (4H, m), 2.70 (3H, s), 2.50 (3H, s).

### Example 7: Synthesis of Compound 205

### Synthesis of Intermediate B54

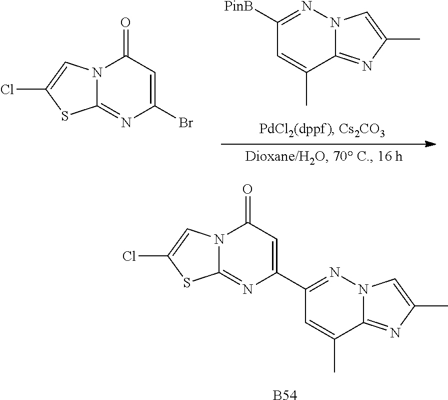

7-bromo-2-chloro-5H-thiazolo[3,2-a]pyrimidin-5-one (100 mg, 0.38 mmol), 2,8-dimethyl-6-(4,4,5,5-tetramethyl-1,3,2-dioxaborolan-2-yl)imidazo[1,2-b]pyridazine (110 mg, 0.40 mmol), PdCl₂(dppf) (30 mg, 0.04 mmol), and Cs₂CO₃ (250 mg, 0.77 mmol) were dissolved in dioxane (5.4 mL) and H₂O (0.5 mL) in a sealed tube and heated at 70° C. for 16 h under argon atmosphere. The reaction mixture was diluted with ethyl acetate (15 mL), and washed with saturated NaHCO₃ (10 mL) and brine (10 mL). The organic phase was dried over Na₂SO₄ and concentrated in vacuo to give a residue. The residue was purified by flash chromatography on a silica gel column using a gradient of 0-25% MeOH in DCM to afford 2-chloro-7-(2,8-dimethylimidazo[1,2-b]pyridazin-6-yl)-5H-thiazolo[3,2-a]pyrimidin-5-one (66 mg, 52%) as a solid. LCMS (ES, m/z): 332.1 [M+H]⁺.

### Synthesis of Compound 205

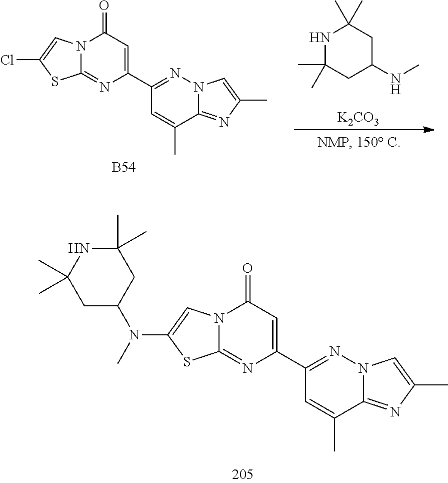

2-chloro-7-(2,8-dimethylimidazo[1,2-b]pyridazin-6-yl)-5H-thiazolo[3,2-a]pyrimidin-5-one (66 mg, 0.20 mmol), K₂CO₃ (85 mg, 0.62 mmol), and N,2,2,6,6-pentamethylpiperidin-4-amine (102 mg, 0.60 mmol) were dissolved in NMP (2 mL) in a sealed tube, and stirred at 150° C. for 5 h. The reaction mixture was cooled to room temperature and diluted with ethyl acetate (25 mL). The organic layer was washed with brine (2×20 mL), dried over anhydrous Na₂SO₄, and concentrated in vacuo to give a residue. The residue was purified by flash chromatography on a silica gel column using a gradient of 0-25% MeOH in DCM to afford 7-(2,8-dimethylimidazo[1,2-b]pyridazin-6-yl)-2-(methyl(2,2,6,6-tetramethylpiperidin-4-yl)amino)-5H-thiazolo[3,2-a]pyrimidin-5-one (16 mg, 18%) as a solid. LCMS (ES, m/z): 466.3 [M+H]⁺. ¹H NMR (DMSO-d₆, 300 MHz): δ 8.12 (1H, s), 7.80 (1H, s), 7.24 (1H, s), 6.98 (1H, s), 2.84 (3H, s), 2.62 (3H, s), 2.42 (3H, s), 1.83 (1H, m), 1.46 (8H, bs), 1.39 (8H, bs).

### Example 8: Synthesis of Compound 206

### Synthesis of Intermediate B55

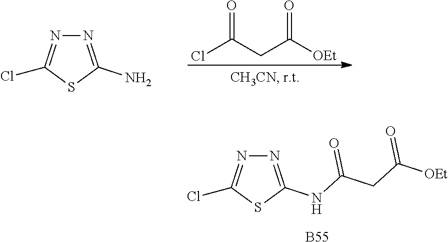

To a solution of 5-chloro-1,3,4-thiadiazol-2-ylamine (500 mg, 3.54 mmol) in anhydrous acetonitrile (12 mL) was added ethyl malonyl chloride (0.82 mL, 6.37 mmol), and the mixture was stirred for 2 days at room temperature in a sealed tube. The reaction mixture was concentrated in vacuo to give a residue, the residue taken up in CH₂Cl₂ (40 mL), and washed with saturated NaHCO₃ (30 mL), water (40 mL), and brine (40 mL). The organic layer was dried over Na₂SO₄, filtered, and concentrated in vacuo to give a residue. The residue was purified on a silica gel cartridge using a gradient of 0-20% EtOAc in CH₂Cl₂ to afford ethyl 3-((5-chloro-1,3,4-thiadiazol-2-yl)amino)-3-oxopropanoate (560 mg, 62%) as a solid. LCMS (ES, m/z): 250.9 [M+H]⁺.

### Synthesis of Intermediate B56

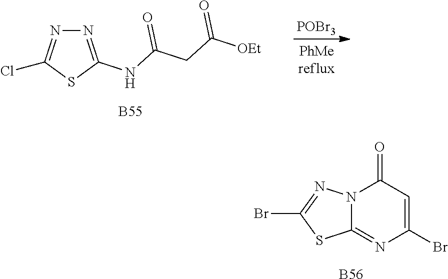 [SMILES: "B55" ; CCOC(=O)CC(=O)Nc1nnc(Cl)s1 ; "B56" ; O=c1cc(Br)nc2sc(Br)nn12 ; "POBr3"]

A mixture of ethyl 3-((5-chloro-1,3,4-thiadiazol-2-yl)amino)-3-oxopropanoate (400 mg, 1.6 mmol), phosphorus(V)oxybromide (2.306 g, 8.04 mmol), and 7 mL toluene in a sealed tube was heated at 120° C. overnight. The reaction mixture was cooled to room temperature and concentrated in vacuo to give a residue. The residue was taken up in CH₂Cl₂, poured over ice, and washed with saturated aqueous NaHCO₃ (50 mL), water (100 mL), and brine (100 mL). The organic layer was dried over Na₂SO₄, and concentrated in vacuo to give a residue. The residue was purified on a silica gel cartridge using a gradient of 0-40% EtOAc in hexane to afford 2,7-dibromo-5H-[1,3,4]thiadiazolo[3,2-a]pyrimidin-5-one (126 mg, 25%) as a solid. LCMS (ES, m/z): 311.8, 313.8 [M+H]⁺.

### Synthesis of Intermediate B57

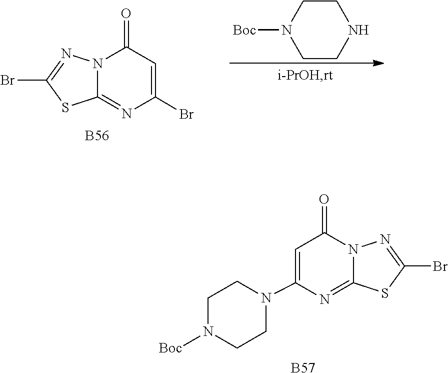 [SMILES: "B56" ; O=c1cc(Br)nc2sc(Br)nn12 ; "B57" ; CC(C)(C)OC(=O)N1CCN(c2cc(=O)n3nc(Br)sc3n2)CC1 ; CC(C)(C)OC(=O)N1CCNCC1]

To a solution of 2,7-dibromo-5H-[1,3,4]thiadiazolo[3,2-a]pyrimidin-5-one (143 mg, 0.46 mmol) in anhydrous isopropyl alcohol (5 mL) was added tert-butyl piperazine-1-carboxylate (170 mg, 0.92 mmol) and the mixture was stirred overnight at room temperature in a sealed tube. The reaction mixture was concentrated in vacuo to give a residue, the residue was taken up in CH₂Cl₂ (25 mL), and washed with saturated NaHCO₃ (25 mL), and water (20 mL). The organic layer was dried over Na₂SO₄, filtered, and concentrated in vacuo to give a residue. The residue was purified on a silica gel cartridge using a gradient of 0-50% EtOAc in hexane to afford tert-butyl 4-(2-bromo-5-oxo-5H-[1,3,4]thiadiazolo[3,2-a]pyrimidin-7-yl)piperazine-1-carboxylate (130 mg, 68%) as a solid. LCMS (ES, m/z): 416.0, 418.0 [M+H]⁺.

### Synthesis of Intermediate B58

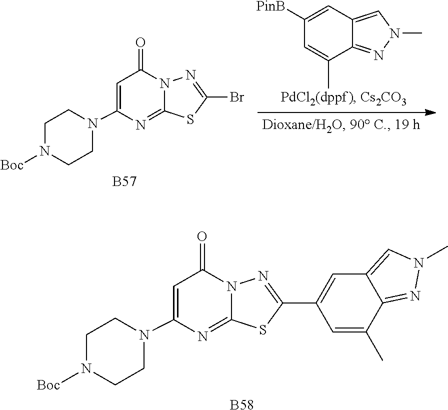

A mixture of tert-butyl 4-(2-bromo-5-oxo-5H-[1,3,4]thiadiazolo[3,2-a]pyrimidin-7-yl)piperazine-1-carboxylate (100 mg, 0.24 mmol), 2,7-dimethyl-5-(4,4,5,5-tetramethyl-1,3,2-dioxaborolan-2-yl)-2H-indazole (83 mg, 0.30 mmol), PdCl₂(dppf)·CH₂Cl₂ (29 mg, 0.04 mmol), and Cs₂CO₃ (185 mg, 0.57 mmol) in a mixture of dioxane (3.0 mL) and H₂O (0.3 mL) was heated at 90° C. for 19 h under a nitrogen atmosphere. The reaction mixture was diluted with ethyl acetate (25 mL) and washed with saturated NaHCO₃ (15 mL) and brine (15 mL). The organic phase was dried over Na₂SO₄ and concentrated in vacuo to give a residue. The residue was purified by flash chromatography on a silica gel column using a gradient of MeOH from 0-10% in DCM to afford tert-butyl 4-(2-(2,7-dimethyl-2H-indazol-5-yl)-5-oxo-5H-[1,3,4]thiadiazolo[3,2-a]pyrimidin-7-yl)piperazine-1-carboxylate (81 mg, 70%) as a solid.

LCMS (ES, m/z): 482.2 [M+H]⁺, Synthesis of Compound 206

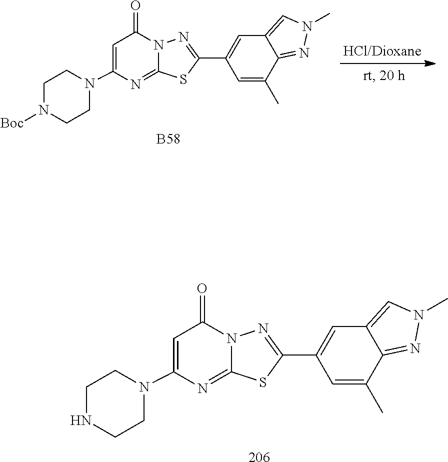 [SMILES: Cc1cc(-c2nn3c(=O)cc(N4CCN(C(=O)OC(C)(C)C)CC4)nc3s2)cc2cn(C)nc12 ; "B58" ; Cc1cc(-c2nn3c(=O)cc(N4CCNCC4)nc3s2)cc2cn(C)nc12 ; "206"]

A mixture of tert-butyl 4-(2-(2,7-dimethyl-2H-indazol-5-yl)-5-oxo-5H-[1,3,4]thiadiazolo[3,2-a]pyrimidin-7-yl)piperazine-1-carboxylate (81 mg, 0.17 mmol) and 4.0 M HCl in dioxane (6.00 mL, 24 mmol), in dioxane (2 mL) was stirred at room temperature for 20 h hours. The reaction mixture was concentrated in vacuo to give a residue, the residue taken up in CH₂Cl₂ (30 mL), and washed with saturated NaHCO₃ (20 mL). The organic layers were combined, dried over Na₂SO₄, filtered, and concentrated in vacuo to give a solid. The solid was purified on a silica gel cartridge using a gradient of MeOH from 0-10% in CH₂Cl₂ to afford 2-(2,7-dimethyl-2H-indazol-5-yl)-7-(piperazin-1-yl)-5H-[1,3,4]thiadiazolo[3,2-a]pyrimidin-5-one (61 mg, 86%) as a solid. LCMS (ES, m/z): 382.1 [M+H]⁺. ¹H NMR (DMSO-d₆, 400 MHz): δ 8.42 (1H, s), 8.31 (1H, s), 7.65 (1H, s), 6.82 (1H, s), 4.17 (3H, s), 3.40 (4H, t, J=4.7 Hz), 2.81 (4H, t, J=4.8 Hz), 2.53 (3H, s).

### Example 9: Synthesis of Compound 159

### Synthesis of Intermediate B59

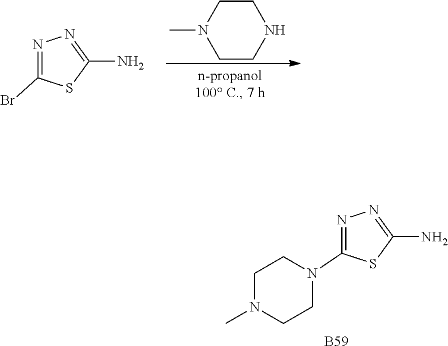

5-Bromo-1,3,4-thiadiazol-2-amine 1 (2.20 g, 12.29 mmol) and 1-methylpiperazine (2.46 g, 24.6 mmol) were dissolved in n-propanol (30 mL) and heated at 100° C. for 7 h in a sealed tube. The reaction mixture was cooled to room temperature, and concentrated in vacuo to give a reside.

The residue was purified on a silica gel cartridge using a gradient of 0-20% MeOH in CH₂Cl₂ containing 1% Et₃N to afford 5-(4-methylpiperazin-1-yl)-1,3,4-thiadiazol-2-amine (825 mg, 63%) as a solid. LCMS (ES, m/z): 200.1 [M+H]⁺.

### Synthesis of Intermediate B60

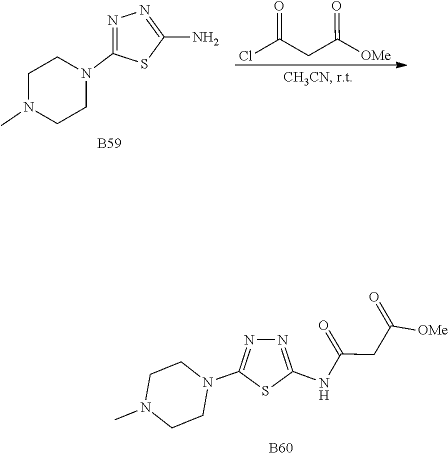 [SMILES: "B59" ; CN1CCN(c2nnc(N)s2)CC1 ; COC(=O)CC(=O)Nc1nnc(N2CCN(C)CC2)s1 ; "B60" ; COC(=O)CC(=O)Cl]

To a solution of 5-(4-methylpiperazin-1-yl)-1,3,4-thiadiazol-2-amine (500 mg, 3.54 mmol) in anhydrous acetonitrile (15 mL) was added ethyl malonyl chloride (0.55 mL, 4.25 mmol). The reaction mixture was stirred for 18 h at room temperature, then concentrated in vacuo to give a residue. The residue was taken up in ethyl acetate (50 mL), poured over ice, and added to saturated NaHCO₃ (40 mL). The mixture was stirred for 20 min, then extracted with ethyl acetate (2×50) and CH₂Cl₂ (3×40). The organic layers was combined, dried over Na₂SO₄, filtered, and concentrated in vacuo to give a solid. The solid was purified on a silica gel cartridge using a gradient of 0-20% MeOH in CH₂Cl₂ to afford methyl 3-((5-(4-methylpiperazin-1-yl)-1,3,4-thiadiazol-2-yl)amino)-3-oxopropanoate (403 mg, 60%) as a solid. LCMS (ES, m/z): 300.1 [M+H]⁺.

### Synthesis of Intermediate B61

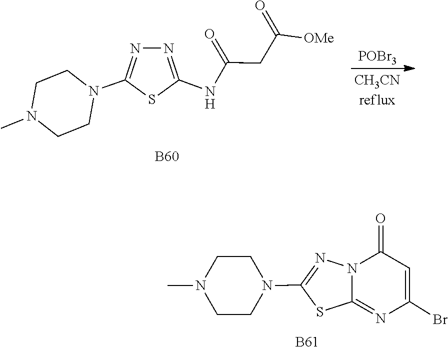

A mixture of methyl 3-((5-(4-methylpiperazin-1-yl)-1,3,4-thiadiazol-2-yl)amino)-3-oxopropanoate (387 mg, 1.3 mmol), phosphorus(V)oxybromide (1.96 g, 6.8 mmol), and acetonitrile (7 mL) was heated at 125° C. overnight. The reaction mixture was cooled to room temperature, concentrated in vacuo to give a residue, and the residue taken up in CH₂Cl₂ (40 mL) and washed with saturated aqueous NaHCO₃ (50 mL), water (100 mL), and brine (50 mL). The organic layer was dried over Na₂SO₄, filtered, and concentrated in vacuo to give a solid. The solid was purified on a silica gel cartridge using a gradient of 0-20% MeOH in CH₂Cl₂ to afford 7-bromo-2-(4-methylpiperazin-1-yl)-5H-[1,3,4]thiadiazolo[3,2-a]pyrimidin-5-one (43 mg, 10%) as a solid. LCMS (ES, m/z): 330.0, 332.0 [M+H]⁺.

### Synthesis of Compound 159

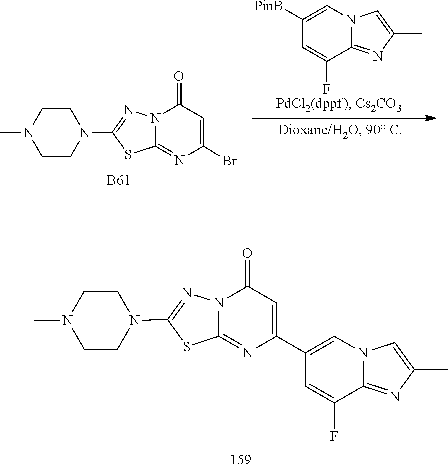

A mixture of 7-bromo-2-(4-methylpiperazin-1-yl)-5H-[1,3,4]thiadiazolo[3,2-a]pyrimidin-5-one (58 mg, 0.18 mmol), 8-fluoro-2-methyl-6-(4,4,5,5-tetramethyl-1,3,2-dioxaborolan-2-yl)imidazo[1,2-a]pyridine (59 mg, 0.214 mmol), PdCl₂(dppf)·CH₂Cl₂ (18 mg, 0.02 mmol) and Cs₂CO₃ (153 mg, 0.47 mmol) in a mixture of dioxane (2.5 mL) and H₂O (0.2 mL) was heated at 90° C. for 18 h under a nitrogen atmosphere. The reaction mixture was diluted with CH₂Cl₂ (50 mL) and washed with saturated aqueous NaHCO₃ (15 mL) and brine (15 mL). The organic phase was dried over Na₂SO₄ and concentrated in vacuo to give a residue. The residue was purified by flash chromatography on a silica gel column using a gradient of MeOH from 0-20% in CH₂Cl₂ to afford 7-(8-fluoro-2-methylimidazo[1,2-a]pyridin-6-yl)-2-(4-methylpiperazin-1-yl)-5H-[1,3,4]thiadiazolo[3,2-a]pyrimidin-5-one (35 mg, 50%) as a solid. LCMS (ES, m/z): 400.1 [M+H]⁺. 1H NMR (CDCl₃, 400 MHz): δ 8.66 (1H, s), 7.47 (1H, s), 7.30 (1H, s), 6.70 (1H, s), 3.64 (4H, s), 2.58 (4H, s), 2.49 (3H, s), 2.39 (3H, s).

### Example 10: Synthesis of Compound 207

### Synthesis of Intermediate B62

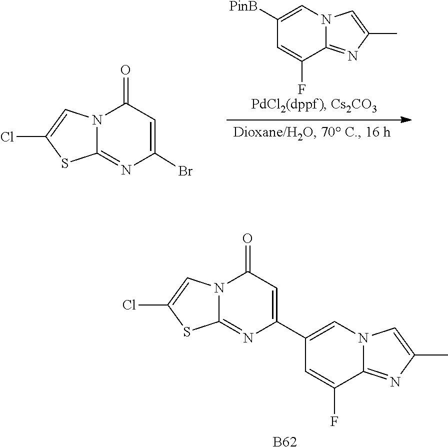

7-bromo-2-chloro-5H-thiazolo[3,2-a]pyrimidin-5-one (100 mg, 0.38 mmol), 8-fluoro-2-methyl-6-(4,4,5,5-tetramethyl-1,3,2-dioxaborolan-2-yl)imidazo[1,2-a]pyridine (110 mg, 0.40 mmol), PdCl₂(dppf) (30 mg, 0.04 mmol), and Cs₂CO₃ (250 mg, 0.77 mmol) were dissolved in a mixture of dioxane (5.4 mL) and H₂O (0.5 mL) and heated at 70° C. for 16 h under argon atmosphere. The reaction mixture was diluted with ethyl acetate (15 mL) and washed with saturated NaHCO₃ (10 mL) and then brine (10 mL). The organic phase was dried over Na₂SO₄ and concentrated in vacuo to give a residue. The residue was purified by flash chromatography on a silica gel column using a gradient of 0-25% MeOH in DCM to afford 2-chloro-7-(8-fluoro-2-methylimidazo[1,2-a]pyridin-6-yl)-SH-thiazolo[3,2-a]pyrimidin-5-one (125 mg, 99%) as a solid. LCMS (ES, m/z): 335.0 [M+H]⁺.

### Synthesis of Compound 207

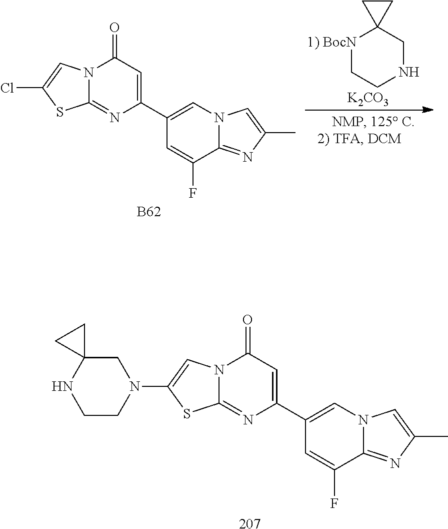

2-chloro-7-(8-fluoro-2-methylimidazo[1,2-a]pyridin-6-yl)-5H-thiazolo[3,2-a]pyrimidin-5-one (75 mg, 0.22 mmol), K₂CO₃ (95 mg, 0.69 mmol), and tert-butyl 4,7-diazaspiro[2.5]octane-4-carboxylate (145 mg, 0.69 mmol) were dissolved in NMP (2.2 mL) in a sealed tube and stirred at 125° C. for 24 h. The reaction mixture was cooled to room temperature and diluted with ethyl acetate (25 mL). The organic layer was washed with brine (2×20 mL), dried over anhydrous Na₂SO₄, filtered, and concentrated in vacuo to give a residue. The residue was purified by flash chromatography on a silica gel column using a gradient of 0-25% MeOH in DCM. Selected fractions were combined and concentrated in vacuo to give a residue. To the residue was added 20% TFA in DCM (3.0 mL) and the solution was stirred at room temperature for 2 h. The reaction mixture was diluted with DCM (20 mL) and washed with 2 N NaOH (2×15 mL). The organic phase was dried over Na₂SO₄, filtered, and concentrated in vacuo to give a residue. The residue was purified by flash chromatography on a silica gel column using a gradient of 0-20% MeOH in DCM to afford 7-(8-fluoro-2-methylimidazo[1,2-a]pyridin-6-yl)-2-(4,7-diazaspiro[2.5]octan-7-yl)-5H-thiazolo[3,2-a]pyrimidin-5-one (12 mg, 14%) as a solid. LCMS (ES, m/z): 411.2 [M+H]⁺. ¹H NMR (DMSO-d₆, 300 MHz): δ 9.18 (1H, s), 7.94 (1H, s), 7.76 (1H, d, J=12.8 Hz), 7.12 (1H, s), 6.86 (1H, s), 5.76 (1H, s), 3.12 (2H, m), 3.01 (2H, s), 2.90 (2H, m), 2.37 (3H, s), 0.54 (4H, d, J=11.9 Hz).

### Example 11: Synthesis of Compound 211

### Synthesis of Compound 211

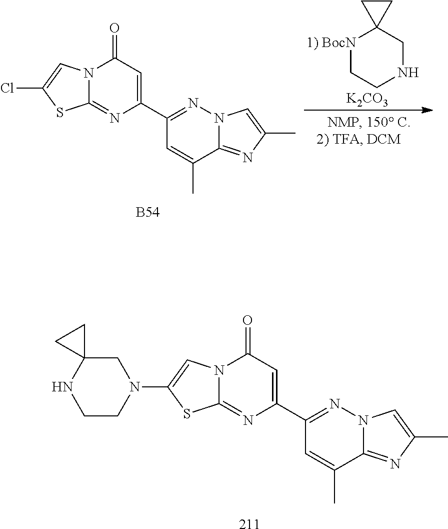

2-chloro-7-(2,8-dimethylimidazo[1,2-b]pyridazin-6-yl)-5H-thiazolo[3,2-a]pyrimidin-5-one (60 mg, 0.18 mmol), K₂CO₃ (126 mg, 0.90 mmol), and tert-butyl 4,7-diazaspiro[2.5]octane-4-carboxylate (115 mg, 0.54 mmol) were dissolved in NMP (900 μL) and stirred at 150° C. for 5 h in a sealed tube. The reaction mixture was cooled to room temperature and diluted with ethyl acetate (25 mL). The organic layer was washed with brine (2×20 mL), dried over anhydrous Na₂SO₄, filtered, and concentrated in vacuo to give a residue. The residue was purified by flash chromatography on a silica gel column using a gradient of 0-25% MeOH in DCM. Selected fractions were combined and concentrated in vacuo to give a residue. To the residue was added 20% TFA in DCM (3.0 mL) and the solution was stirred at room temperature for 2 h. The reaction mixture was diluted with DCM (20 mL) and washed with 2 N NaOH (2×15 mL). The organic phase was dried over Na₂SO₄, filtered, and concentrated in vacuo to give a reside. The residue was purified by flash chromatography on a silica gel column using a gradient of 0-20% MeOH in DCM to afford 7-(2,8-dimethylimidazo[1,2-b]pyridazin-6-yl)-2-(4,7-diazaspiro[2.5]octan-7-yl)-5H-thiazolo[3,2-a]pyrimidin-5-one (9.2 mg, 12%) as a solid. LCMS (ES, m/z): 408.2 [M+H]⁺. ¹H NMR (DMSO-d₆, 300 MHz): δ 8.12 (1H, s), 7.81 (1H, s), 7.19 (1H, s), 6.96 (1H, s), 3.15 (2H, m), 3.05 (2H, s), 2.91 (2H, m), 2.61 (3H, s), 2.42 (3H, s), 0.54 (4H, d, J=13.8 Hz).

### Example 12: Synthesis of Compound 208

### Synthesis of Intermediate B55

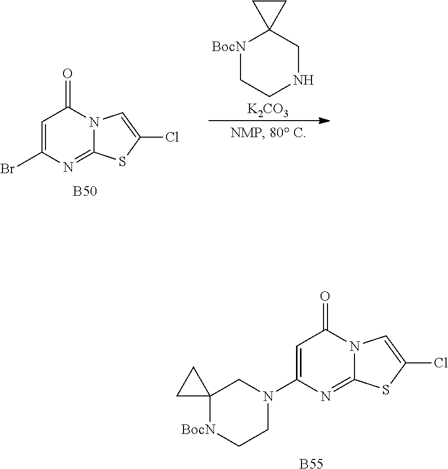

7-bromo-2-chloro-5H-thiazolo[3,2-a]pyrimidin-5-one (85 mg, 0.32 mmol), K₂CO₃ (223 mg, 1.60 mmol), and tert-butyl 4,7-diazaspiro[2.5]octane-4-carboxylate (204 mg, 0.96 mmol) were dissolved in NMP (1.6 mL) and stirred at 80° C. for 4 h in a sealed tube. The reaction mixture was cooled to room temperature and diluted with ethyl acetate (25 mL). The organic layer was washed with brine (2×20 mL), dried over anhydrous Na₂SO₄, and concentrated in vacuo to give a residue. The residue was purified by flash chromatography on a silica gel column using a gradient of 0-100% ethyl acetate in hexane to afford 7-(2-chloro-5-oxo-5H-thiazolo[3,2-a]pyrimidin-7-yl)-4,7-diazaspiro[2.5]octane-4-carboxylate (107 mg, 84%) as a solid. LCMS (ES, m/z): 397.1 [M+H]⁺.

### Synthesis of Compound 208

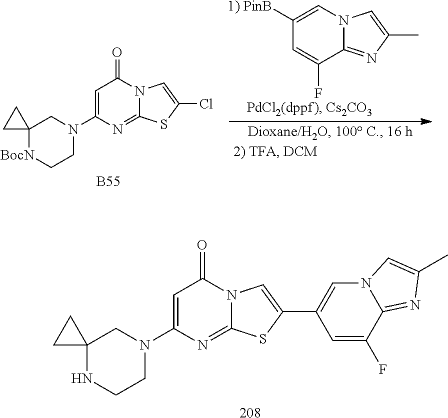

7-(2-chloro-5-oxo-5H-thiazolo[3,2-a]pyrimidin-7-yl)-4,7-diazaspiro[2.5]octane-4-carboxylate (50 mg, 0.13 mmol), 8-fluoro-2-methyl-6-(4,4,5,5-tetramethyl-1,3,2-dioxaborolan-2-yl)imidazo[1,2-a]pyridine (42 mg, 0.15 mmol), PdCl₂(dppf) (9.2 mg, 0.013 mmol) and Cs₂CO₃ (123 mg, 0.38 mmol) were dissolved in a mixture of dioxane (1.1 mL) and H₂O (110 μL) and heated at 100° C. for 16 h under argon atmosphere. The reaction mixture was diluted with ethyl acetate (15 mL), then washed with saturated NaHCO₃ (10 mL) and brine (10 mL). The organic phase was dried over Na₂SO₄, filtered, and concentrated in vacuo to give a residue. The residue was purified by flash chromatography on a silica gel column using a gradient of 25-100% ethyl acetate in DCM. Selected fractions were combined and concentrated in vacuo to give a reside.

To the residue was added 20% TFA in DCM (3.0 mL) and the solution was stirred at room temperature for 2 h. The reaction mixture was diluted with DCM (20 mL) and washed with 2 N NaOH (2×15 mL). The organic phase was dried over Na₂SO₄, filtered, and concentrated in vacuo to give a residue. The residue was purified by flash chromatography on a silica gel column using a gradient of 0-20% MeOH in DCM to afford 2-(8-fluoro-2-methylimidazo[1,2-a]pyridin-6-yl)-7-(4,7-diazaspiro[2.5]octan-7-yl)-5H-thiazolo[3,2-a]pyrimidin-5-one (28 mg, 54%) as a solid. LCMS (ES, m/z): 411.1 [M+H]⁺. ¹H NMR (DMSO-d₆, 300 MHz): δ 8.72 (1H, s), 8.43 (1H, s), 7.83 (1H, s), 7.80 (1H, s), 7.76 (1H, m), 5.35 (1H, s), 3.54 (2H, m), 3.41 (2H, s), 2.80 (2H, m), 2.37 (3H, s), 0.48 (4H, d, J=17.4 Hz).

### Example 13: Synthesis of Compound 227

### Synthesis of Intermediate B56

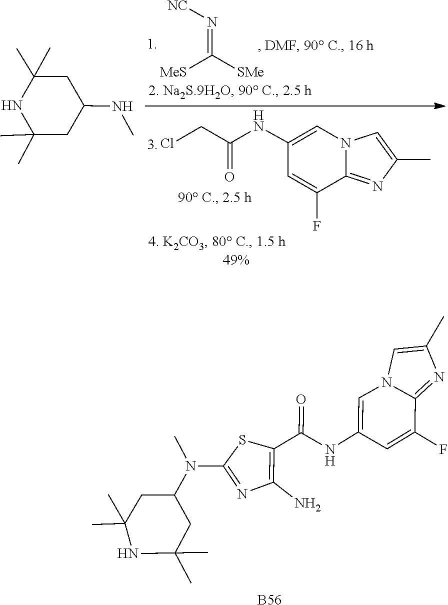

To a solution of N,2,2,6,6-pentamethylpiperidin-4-amine (30 mg, 0.18 mmol) in DMF (1.0 mL) was added dimethyl-N-cyanodithioiminocarbonate (26 mg, 0.18 mmol). The reaction mixture was heated to 90° C. for 16 h. Na₂S·9H₂O (43 mg, 0.18 mmol) was added and the reaction mixture was stirred at 90° C. for 2.5 h. A solution of 2-chloro-N-(8-fluoro-2-methylimidazo[1,2-a]pyridin-6-yl)acetamide (0.12 g, 0.52 mmol) in DMF (1.0 mL) was added and the reaction mixture was stirred at 90° C. for 2.5 h. K₂CO₃ (30 mg, 0.21 mmol) was added and the reaction mixture was stirred at 80° C. for 1.5 h. The reaction mixture was filtered, and concentrated in vacuo to give a residue. The residue was purified by reverse phase chromatography (C18) using 0-100% acetonitrile in water followed by 100% MeOH to afford 4-amino-N-(8-fluoro-2-methylimidazo[1,2-a]pyridin-6-yl)-2-(methyl(2,2,6,6-tetramethylpiperidin-4-yl)amino)thiazole-5-carboxamide (40 mg, 49%) as a solid. LCMS (ES, m/z): 460.0 [M+H]⁺.

### Synthesis of Compound 227

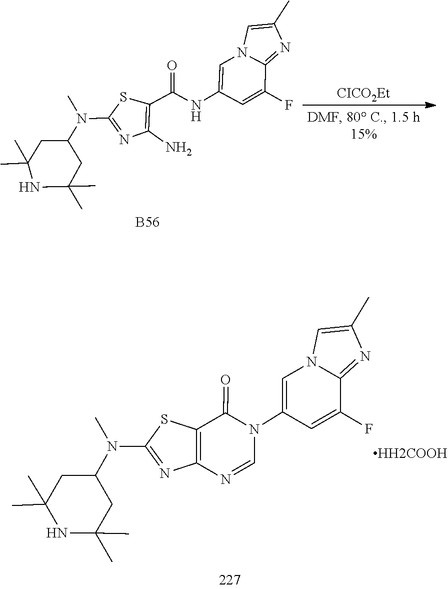 [SMILES: "B56" ; Cc1cn2cc(NC(=O)c3sc(N(C)C4CC(C)(C)NC(C)(C)C4)nc3N)cc(F)c2n1 ; Cc1cn2cc(-n3cnc4nc(N(C)C5CC(C)(C)NC(C)(C)C5)sc4c3=O)cc(F)c2n1 ; "CICO2Et"]

To a solution of 4-amino-N-(8-fluoro-2-methylimidazo[1,2-a]pyridin-6-yl)-2-(methyl(2,2,6,6-tetramethylpiperidin-4-yl)amino)thiazole-5-carboxamide (40 mg, 0.087 mmol) in DMF (3.0 mL) was added ethyl chloroformate (0.15 mL, 3.2 mmol). The reaction mixture was stirred at room temperature for 10 min, then at 80° C. for 90 min. The volatiles were evaporated under reduced pressure. A saturated solution of NaHCO₃ (10 mL) and DCM (15 mL) were added, and the layers were separated. The aqueous layer was extracted with DCM (3×15 mL). The organic layers were combined, dried over Na₂SO₄, filtered, and concentrated in vacuo to give a residue. The residue was purified by reverse phase chromatography (C18) using a gradient of 0-10% acetonitrile in water (containing 0.1% FA) to afford 6-(8-fluoro-2-methylimidazo[1,2-a]pyridin-6-yl)-2-(methyl(2,2,6,6-tetramethylpiperidin-4-yl)amino)thiazolo[4,5-d]pyrimidin-7(6H)-one, formate (6.5 mg, 15%) as a formic acid salt. LCMS (ES, m/z): 470.2 [M+H]⁺. ¹H NMR (DMSO-d₆, 400 MHz): δ_{H} 8.74 (1H, s), 8.46 (1H, s), 8.33 (1H, s), 7.92 (1H, s), 7.38 (1H, d, J=11.5 Hz), 3.05 (4H, s), 2.39 (3H, s), 1.60 (2H, m), 1.48 (2H, m), 1.24 (6H, s), 1.11 (6H, s).

### Example 14: Synthesis of Compound 209

### Synthesis of Intermediate B57

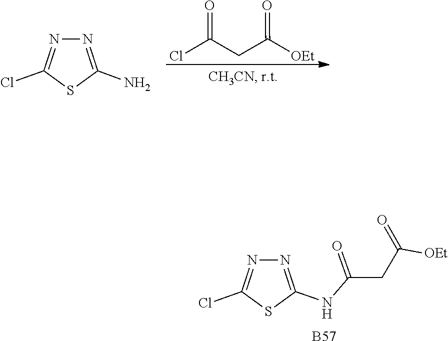 [SMILES: Nc1nnc(Cl)s1 ; CCOC(=O)CC(=O)Cl ; "B57" ; CCOC(=O)CC(=O)Nc1nnc(Cl)s1]

To a solution of 5-chloro-1,3,4-thiadiazol-2-ylamine (500 mg, 3.54 mmol) in anhydrous acetonitrile (12 mL) was added the ethyl malonyl chloride (0.82 mL, 6.37 mmol). The reaction mixture was stirred for 2 days at room temperature in a sealed tube, then concentrated in vacuo to give a residue, the residue taken up in CH₂Cl₂ (40 mL), and washed with saturated aqueous NaHCO₃ (30 mL), water (40 mL), and brine (40 mL). The organic layer was dried over Na₂SO₄, filtered, and concentrated in vacuo to give a solid. The solid was purified on a silica gel cartridge using a gradient of 0-20% EtOAc in CH₂Cl₂ to afford ethyl 3-((5-chloro-1,3,4-thiadiazol-2-yl)amino)-3-oxopropanoate (560 mg, 62%) as a solid. LCMS (ES, m/z): 250.9 [M+H]⁺.

### Synthesis of Intermediate B58

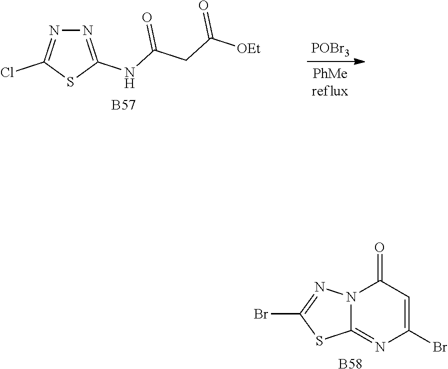 [SMILES: "B57" ; CCOC(=O)CC(=O)Nc1nnc(Cl)s1 ; "B58" ; O=c1cc(Br)nc2sc(Br)nn12 ; "POBr3"]

A mixture of ethyl 3-((5-chloro-1,3,4-thiadiazol-2-yl)amino)-3-oxopropanoate (400 mg, 1.6 mmol), phosphorus(V)oxybromide (2.306 g, 8.04 mmol), and toluene (7 mL) was heated at 120° C. overnight in a sealed tube. The reaction mixture was cooled to room temperature, concentrated in vacuo to a residue, and the residue taken up in CH₂Cl₂, poured over ice, and washed with saturated aqueous NaHCO₃ (50 mL), water (100 mL), and brine (100 mL). The organic layer was dried over Na₂SO₄, filtered, and concentrated in vacuo to give a solid. The solid was purified on a silica gel cartridge using a gradient of 0-40% EtOAc in hexane to afford 2,7-dibromo-5H-[1,3,4]thiadiazolo[3,2-a]pyrimidin-5-one (126 mg, 25%) as a solid. LCMS (ES, m/z): 309.8, 311.8 [M+H]⁺.

### Synthesis of Intermediate B59

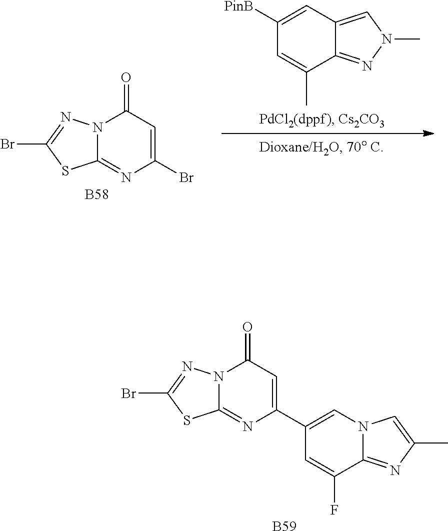

A mixture of 2,7-dibromo-5H-[1,3,4]thiadiazolo[3,2-a]pyrimidin-5-one (205 mg, 0.66 mmol), 8-fluoro-2-methyl-6-(4,4,5,5-tetramethyl-1,3,2-dioxaborolan-2-yl)imidazo[1,2-a]pyridine (199 mg, 0.72 mmol), PdCl₂(dppf)·CH₂Cl₂ (54 mg, 0.07 mmol), and Cs₂CO₃ (436 mg, 1.34 mmol) in a mixture of dioxane (9.2 mL) and H₂O (0.8 mL) was heated at 70° C. for 19 h under nitrogen atmosphere in a sealed tube. The reaction mixture was diluted with ethyl acetate (20 mL) and washed with saturated aqueous NaHCO₃ (15 mL) and brine (15 mL). The organic phase was dried over Na₂SO₄, filtered, and concentrated in vacuo to give a residue. The residue was purified by flash chromatography on silica gel column using a gradient of ethyl acetate from 30-60% in CH₂Cl₂ to afford 2-bromo-7-(8-fluoro-2-methylimidazo[1,2-a]pyridin-6-yl)-5H-[1,3,4]thiadiazolo[3,2-a]pyrimidin-5-one (90 mg, 36%) as a solid. LCMS (ES, m/z): 379.9, 381.9 [M+H]⁺.

### Synthesis of Intermediate B60

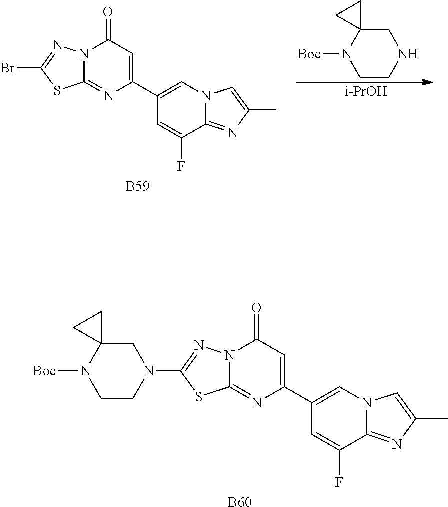

To a solution of 2-bromo-7-(8-fluoro-2-methylimidazo[1,2-a]pyridin-6-yl)-5H-[1,3,4]thiadiazolo[3,2-a]pyrimidin-5-one (62 mg, 0.163 mmol) in anhydrous isopropyl alcohol (3 mL) was added tert-butyl 4,7-diazaspiro[2.5]octane-4-carboxylate (76 mg, 0.358 mmol). The reaction mixture was stirred at 100° C. for 7 h in a sealed tube. The reaction mixture was concentrated in vacuo to give a residue, the residue taken up in CH₂Cl₂ (30 mL), and washed with saturated aqueous NaHCO₃ (15 mL), and brine (15 mL). The organic layer was dried over Na₂SO₄, filtered, and concentrated in vacuo to give a residue. The residue was purified on a silica gel cartridge using a gradient of 0-10% MeOH in CH₂Cl₂ to afford tert-butyl 7-(7-(8-fluoro-2-methylimidazo[1,2-a]pyridin-6-yl)-5-oxo-5H-[1,3,4]thiadiazolo[3,2-a]pyrimidin-2-yl)-4,7-diazaspiro[2.5]octane-4-carboxylate (64 mg, 77%) as a solid. LCMS (ES, m/z): 512.2 [M+H]⁺.

### Synthesis of Compound 209

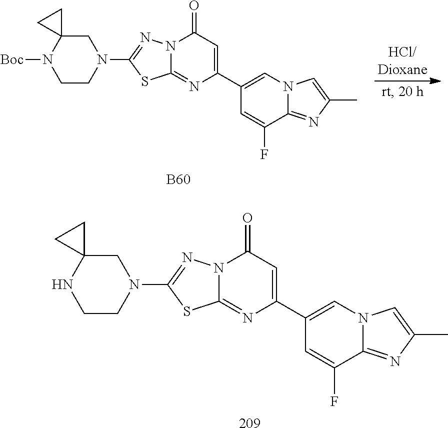

A mixture of tert-butyl 7-(7-(8-fluoro-2-methylimidazo[1,2-a]pyridin-6-yl)-5-oxo-5H-[1,3,4]thiadiazolo[3,2-a]pyrimidin-2-yl)-4,7-diazaspiro[2.5]octane-4-carboxylate (55 mg, 0.11 mmol) and 4.0 M HCl in dioxane (3.4 mL, 12.90 mmol), in dioxane (2 mL) was stirred at room temperature for 24 hours in a sealed tube. The reaction mixture was concentrated in vacuo to give a residue, the residue taken up in CH₂Cl₂ (40 mL), and washed with saturated aqueous NaHCO₃ (20 mL). The organic layer was dried over Na₂SO₄, filtered, and concentrated in vacuo to give a solid. The solid was purified on a silica gel cartridge using a gradient of MeOH from 0-20% in CH₂Cl₂ to afford 7-(8-fluoro-2-methylimidazo[1,2-a]pyridin-6-yl)-2-(4,7-diazaspiro[2.5]octan-7-yl)-5H-[1,3,4]thiadiazolo[3,2-a]pyrimidin-5-one (31 mg, 70%) as a solid. LCMS (ES, m/z): 412.1 [M+H]⁺. ¹H NMR (DMSO-d₆, 400 MHz): δ 9.11 (1H, s), 7.85 (1H, s), 7.49 (1H, d, J=11.3 Hz), 5.41 (1H, s), 3.60 (2H, s), 3.49 (2H, s), 2.93 (2H, s), 2.39 (3H, s), 0.60 (4H, s).

### Example 15: Synthesis of Compound 210

### Synthesis of Compound 210

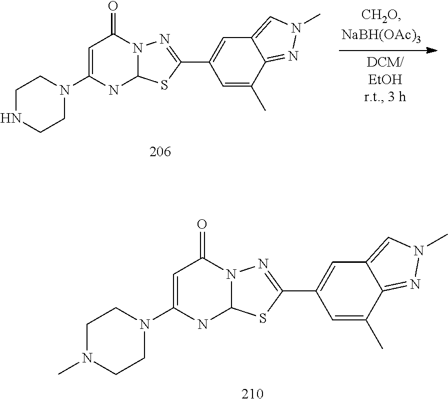

2-(2,7-dimethyl-2H-indazol-5-yl)-7-(piperazin-1-yl)-5H-[1,3,4]thiadiazolo[3,2-a]pyrimidin-5-one (44 mg, 0.12 mmol) was dissolved in a mixture of CH₂Cl₂ (1.5 mL) and EtOH (0.5 mL). To this solution was added formaldehyde (37% in water, 0.43 mL, 0.58 mmol). The reaction mixture was stirred at room temperature for 1 h in a sealed tube. Then NaBH(OAc)₃ (147 mg, 0.69 mmol) was added and the reaction mixture was stirred for an additional 2 h at room temperature. The reaction mixture was concentrated in vacuo to give a residue, diluted with CH₂Cl₂ (30 mL), then washed with saturated NaHCO₃ (20 mL), and brine (20 mL). The organic layer was dried over Na₂SO₄, filtered, and concentrated in vacuo to give a residue. The residue was purified by flash chromatography on silica gel column using a gradient of 0-20% MeOH in CH₂Cl₂ to afford 2-(2,7-dimethyl-2H-indazol-5-yl)-7-(4-methylpiperazin-1-yl)-5H-[1,3,4]thiadiazolo[3,2-a]pyrimidin-5-one (41 mg, 90%) as a solid. LCMS (ES, m/z): 396.1 [M+H]⁺. 1H NMR (CDCl₃, 400 MHz): δ 8.17 (1H, s), 7.98 (1H, s), 7.58 (1H, s), 6.85 (1H, s), 4.25 (3H, m), 3.68 (4H, s), 2.67 (7H, m), 2.43 (3H, s).

### Example 16: Synthesis of Compound 228

### Synthesis of Intermediate B61

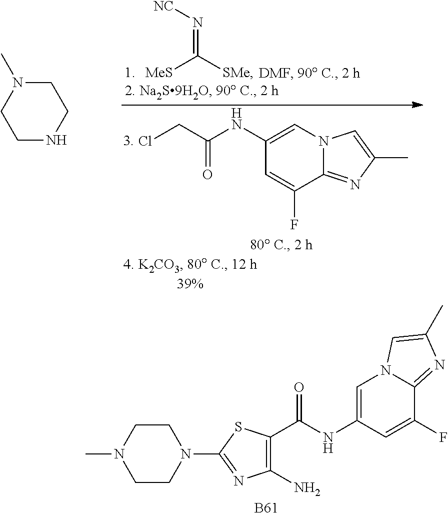

To a solution of N-methyl piperazine (50 mg, 0.50 mmol) in DMF (1.5 mL) was added dimethyl-N-cyanodithioiminocarbonate (73 mg, 0.50 mmol). The reaction mixture was heated to 90° C. for 2 h. Na₂S·9H₂O (120 mg, 0.50 mmol) was added and the reaction mixture was stirred at 90° C. for an additional 2 h. A solution of 2-chloro-N-(8-fluoro-2-methylimidazo[1,2-a]pyridin-6-yl)acetamide (0.34 g, 1.5 mmol) in DMF (1.5 mL) was added dropwise and the reaction mixture was stirred at 80° C. for 2 h. K₂CO₃ (69 mg, 0.50 mmol) was added and the reaction mixture was stirred at 80° C. for 12 h. The reaction mixture was filtered, and concentrated in vacuo to give a residue. The residue was purified by reverse phase chromatography (C18) using 0-100% acetonitrile in water to afford 4-amino-N-(8-fluoro-2-methylimidazo[1,2-a]pyridin-6-yl)-2-(4-methylpiperazin-1-yl)thiazole-5-carboxamide (76 mg, 39%) as a solid. LCMS (ES, m/z): 389.9 [M+H]⁺.

### Synthesis of Compound 228

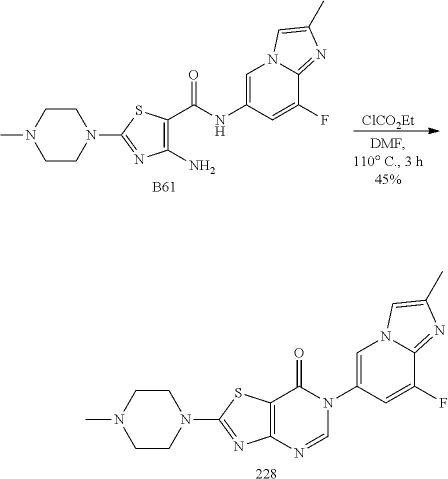

To a solution of 4-amino-N-(8-fluoro-2-methylimidazo[1,2-a]pyridin-6-yl)-2-(4-methylpiperazin-1-yl)thiazole-5-carboxamide (75 mg, 0.19 mmol) in DMF (2.5 mL) was added ethyl chloroformate (0.37 mL, 3.8 mmol). The reaction mixture was heated to 110° C. for 3 h, then cooled to room temperature. The reaction mixture was concentrated in vacuo. A saturated solution of NaHCO₃ (20 mL) and DCM (20 mL) were added, and the layers were separated. The aqueous layer was extracted with DCM (3×20 mL). The organic layers were combined, dried over Na₂SO₄, filtered, and concentrated in vacuo to give a residue. The residue was purified by column chromatography on silica gel using a gradient of 0-10% MeOH in DCM to afford 6-(8-fluoro-2-methylimidazo[1,2-a]pyridin-6-yl)-2-(4-methylpiperazin-1-yl)thiazolo[4,5-d]pyrimidin-7(6H)-one (35 mg, 45%) as a solid. LCMS (ES, m/z): 400.1 [M+H]⁺. ¹H NMR (DMSO-d₆, 400 MHz): δ_{H} 8.73 (1H, s), 8.45 (1H, s), 7.90 (1H, s), 7.39 (1H, d, J=11.6 Hz), 3.63 (4H, s), 2.38 (3H, s), 2.25 (3H, s).

### Example 17: Synthesis of Compound 212

### Synthesis of Compound 212

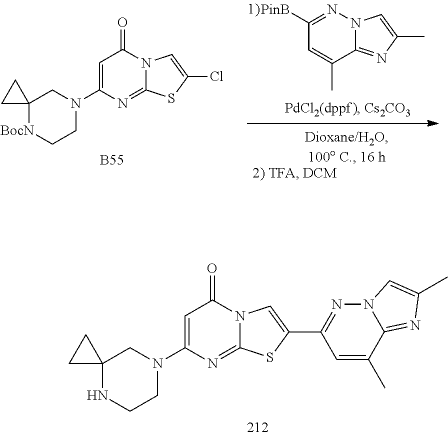

7-(2-chloro-5-oxo-5H-thiazolo[3,2-a]pyrimidin-7-yl)-4,7-diazaspiro[2.5]octane-4-carboxylate (55 mg, 0.139 mmol), 2,8-dimethyl-6-(4,4,5,5-tetramethyl-1,3,2-dioxaborolan-2-yl)imidazo[1,2-b]pyridazine (68 mg, 0.25 mmol), PdCl₂(dppf) (10 mg, 0.014 mmol), and Cs₂CO₃ (135 mg, 0.42 mmol) were dissolved in a mixture of dioxane (1.8 mL) and H₂O (180 μL) and heated at 100° C. for 16 h under argon atmosphere in a sealed tube. The reaction mixture was diluted with ethyl acetate (15 mL), then washed with saturated NaHCO₃ (10 mL) and brine (10 mL). The organic phase was dried over Na₂SO₄, filtered, and concentrated in vacuo to give a residue. The residue was purified by flash chromatography on a silica gel column using a gradient of 25-100% ethyl acetate in DCM. Selected fractions were combined and concentrated in vacuo to give a reside.

To the residue was added 20% TFA in DCM (3.0 mL) and the solution was stirred at room temperature for 2 h. The reaction mixture was diluted with DCM (20 mL) and washed with 2 N NaOH (2×15 mL). The organic phase was dried over Na₂SO₄, filtered, and concentrated in vacuo to give a residue. The residue was purified by flash chromatography on a silica gel column using a gradient of 0-20% MeOH in DCM to afford 2-(2,8-dimethylimidazo[1,2-b]pyridazin-6-yl)-7-(4,7-diazaspiro[2.5]octan-7-yl)-5H-thiazolo[3,2-a]pyrimidin-5-one (8.9 mg, 16%) as a solid. LCMS (ES, m/z): 408.2 [M+H]⁺. ¹H NMR (DMSO-d₆, 300 MHz): δ 8.79 (1H, s), 8.06 (1H, s), 7.99 (1H, s), 5.36 (1H, s), 3.56 (1H, m), 3.43 (2H, s), 3.30 (2H, s), 2.76 (2H, m), 2.56 (3H, s), 2.39 (3H, s), 0.52 (2H, m), 0.45 (2H, m).

### Example 18: Synthesis of Compound 157

### Synthesis of Compound 157

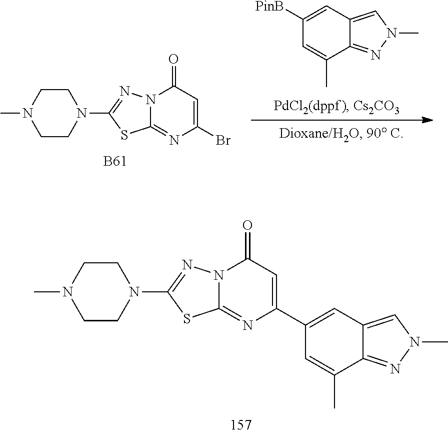

A mixture of 7-bromo-2-(4-methylpiperazin-1-yl)-5H-[1,3,4]thiadiazolo[3,2-a]pyrimidin-5-one (58 mg, 0.18 mmol), 2,7-dimethyl-5-(3,3,4,4-tetramethylborolan-1-yl)-2H-indazole (107 mg, 0.37 mmol), PdCl₂(dppf) (35 mg, 0.04 mmol) and Cs₂CO₃ (229 mg, 0.69 mmol) in a mixture of dioxane (2.4 mL) and H₂O (0.25 mL) was heated at 90° C. for 16 h under nitrogen atmosphere in a sealed tube. The reaction mixture was diluted with CH₂Cl₂ (30 mL) and washed with saturated aqueous NaHCO₃ (15 mL) and brine (20 mL). The organic phase was dried over Na₂SO₄, filtered, and concentrated in vacuo to give a residue. The residue was purified by flash chromatography on a silica gel column using a gradient of ethyl acetate from 30 to 60% in CH₂Cl₂ to afford 7-(2,7-dimethyl-2H-indazol-5-yl)-2-(4-methylpiperazin-1-yl)-5H-[1,3,4]thiadiazolo[3,2-a]pyrimidin-5-one (32 mg, 32%) as a solid. LCMS (ES, m/z): 395.8 [M+H]⁺. 1H NMR (CDCl₃, 400 MHz): δ 8.16 (1H, s), 7.98 (1H, s), 7.57 (1H, s), 6.84 (1H, s), 4.25 (3H, s), 3.65 (4H, br s), 2.67 (3H, s), 2.61 (4H, br s), 2.40 (3H, s).

### Example 19: Synthesis of Compound 213

### Synthesis of Intermediate B64

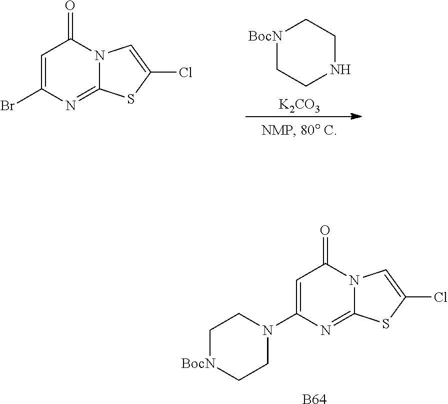 [SMILES: O=c1cc(Br)nc2sc(Cl)cn12 ; "K2CO3" ; CC(C)(C)OC(=O)N1CCNCC1 ; "B64" ; CC(C)(C)OC(=O)N1CCN(c2cc(=O)n3cc(Cl)sc3n2)CC1]

7-bromo-2-chloro-5H-thiazolo[3,2-a]pyrimidin-5-one (100 mg, 0.38 mmol), K₂CO₃ (263 mg, 1.88 mmol), and tert-butyl piperazine-1-carboxylate (210 mg, 1.13 mmol) were dissolved in NMP (1.9 mL) and stirred at 80° C. for 4 h. The reaction mixture was cooled to room temperature and diluted with ethyl acetate (25 mL). The organic layer was washed with brine (2×20 mL), dried over anhydrous Na₂SO₄, filtered, and concentrated in vacuo to give a residue. The residue was purified by flash chromatography on a silica gel column using a gradient of 0-100% ethyl acetate in hexane to afford tert-butyl 4-(2-chloro-5-oxo-5H-thiazolo[3,2-a]pyrimidin-7-yl)piperazine-1-carboxylate (107 mg, 76%) as a solid. LCMS (ES, m/z): 371.1 [M+H]⁺.

### Synthesis of Compound 213

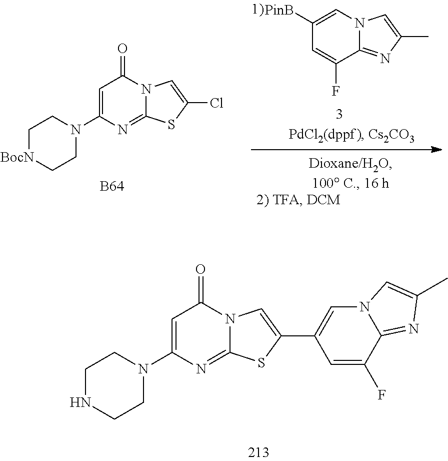

Tert-butyl 4-(2-chloro-5-oxo-5H-thiazolo[3,2-a]pyrimidin-7-yl)piperazine-1-carboxylate (53 mg, 0.14 mmol), 8-fluoro-2-methyl-6-(4,4,5,5-tetramethyl-1,3,2-dioxaborolan-2-yl)imidazo[1,2-a]pyridine (47 mg, 0.17 mmol), PdCl₂(dppf) (11 mg, 0.014 mmol) and Cs₂CO₃ (140 mg, 0.43 mmol) were dissolved in dioxane (1.3 mL) and H₂O (130 μL) and heated at 100° C. for 16 h under argon atmosphere in a sealed tube. The reaction mixture was diluted with ethyl acetate (15 mL), then washed with saturated NaHCO₃ (10 mL) and brine (10 mL). The organic phase was dried over Na₂SO₄, filtered, and concentrated in vacuo to give a residue. The residue was purified by flash chromatography on a silica gel column using a gradient of 50-100% ethyl acetate in DCM. Selected fractions were combined and concentrated in vacuo to give a residue. To the residue was added 20% TFA in DCM (3.0 mL) and the solution was stirred at room temperature for 2 h. The reaction mixture was diluted with DCM (20 mL) and washed with 2 N NaOH (2×15 mL). The organic phase was dried over Na₂SO₄, filtered, and concentrated in vacuo to afford 2-(8-fluoro-2-methylimidazo[1,2-a]pyridin-6-yl)-7-(piperazin-1-yl)-5H-thiazolo[3,2-a]pyrimidin-5-one (13 mg, 24%) as a solid. LCMS (ES, m/z): 385.1 [M+H]⁺. ¹H NMR (DMSO-d₆, 300 MHz): δ 8.73 (1H, s), 8.45 (1H, s), 7.82 (2H, m), 5.35 (1H, s), 3.49 (4H, m), 2.74 (4H, m), 2.37 (3H, s).

### Example 20: Synthesis of Compound 214

### Synthesis of Intermediate B65

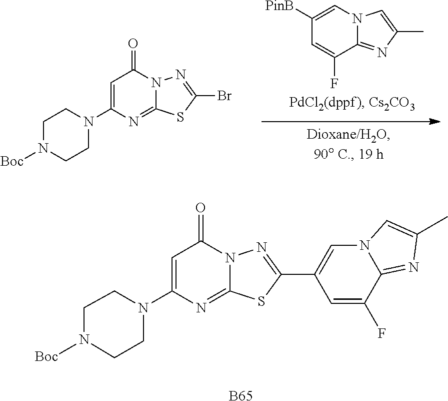

A mixture of tert-butyl 4-(2-bromo-5-oxo-5H-[1,3,4]thiadiazolo[3,2-a]pyrimidin-7-yl)piperazine-1-carboxylate (135 mg, 0.32 mmol), 8-fluoro-2-methyl-6-(3,3,4,4-tetramethylborolan-1-yl)imidazo[1,2-a]pyridine (101 mg, 0.36 mmol), PdCl₂(dppf) (24 mg, 0.03 mmol), and Cs₂CO₃ (211 mg, 0.65 mmol) in a mixture of dioxane (3.0 mL) and H₂O (0.3 mL) was heated at 90° C. for 16 h under nitrogen atmosphere in a sealed tube. The reaction mixture was concentrated in vacuo to give a residue, the residue taken up in CH₂Cl₂ (25 mL), washed with saturated aqueous NaHCO₃ (15 mL) and brine (15 mL). The organic phase was dried over Na₂SO₄, filtered, and concentrated in vacuo to give a residue. The residue was purified by flash chromatography on a silica gel column using a gradient of methanol from 0 to 10% in CH₂Cl₂ to afford tert-butyl 4-(2-(8-fluoro-2-methylimidazo[1,2-a]pyridin-6-yl)-5-oxo-5H-[1,3,4]thiadiazolo[3,2-a]pyrimidin-7-yl)piperazine-1-carboxylate (139 mg, 88%) as a solid.

LCMS (ES, m/z): 486.5 [M+H]⁺.

### Synthesis of Compound 214

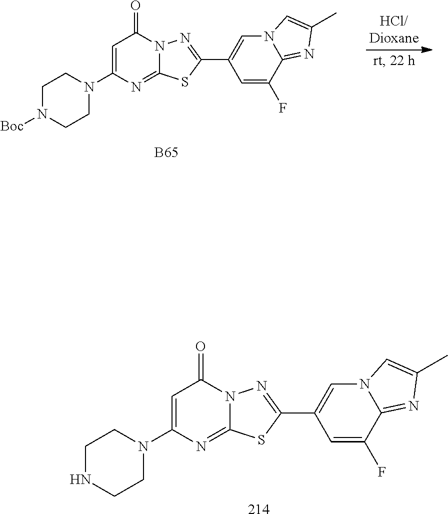 [SMILES: "B65" ; Cc1cn2cc(-c3nn4c(=O)cc(N5CCN(C(=O)OC(C)(C)C)CC5)nc4s3)cc(F)c2n1 ; Cc1cn2cc(-c3nn4c(=O)cc(N5CCNCC5)nc4s3)cc(F)c2n1 ; "214"]

A mixture of tert-butyl 4-(2-(8-fluoro-2-methylimidazo[1,2-a]pyridin-6-yl)-5-oxo-5H-[1,3,4]thiadiazolo[3,2-a]pyrimidin-7-yl)piperazine-1-carboxylate (129 mg, 0.27 mmol) and HCl in dioxane (4 M, 8.00 mL, 32 mmol), in dioxane (13 mL) was stirred at room temperature for 22 h. The reaction mixture was concentrated in vacuo to give a residue, the residue taken up in CH₂Cl₂ (40 mL), and washed with saturated aqueous NaHCO₃ (20 mL). The aqueous phase was concentrated in vacuo to give a reside, and the residue was extracted with methanol. The organic layers were combined, dried over Na₂SO₄, filtered, and concentrated in vacuo to give a solid. The solid was purified on a silica gel cartridge using a gradient of methanol from 0 to 20% in CH₂Cl₂ to afford 2-(8-fluoro-2-methylimidazo[1,2-a]pyridin-6-yl)-7-(piperazin-1-yl)-5H-[1,3,4]thiadiazolo[3,2-a]pyrimidin-5-one (78 mg, 76%) as a solid. LCMS (ES, m/z): 385.8 [M+H]⁺. 1H NMR (CH₃OH-d₄ (0.75 mL)+CDCl₃ (0.25 mL), 400 MHz): δ 8.91 (1H, s), 7.68 (1H, s), 7.55 (1H, d, J=11.8 Hz), 6.81 (1H, s), 3.57 (4H, s), 3.34 (1H, s, NH), 2.98 (4H, s), 2.43 (3H, s).

### Example 21: Synthesis of Compound 215

### Synthesis of Intermediate B66

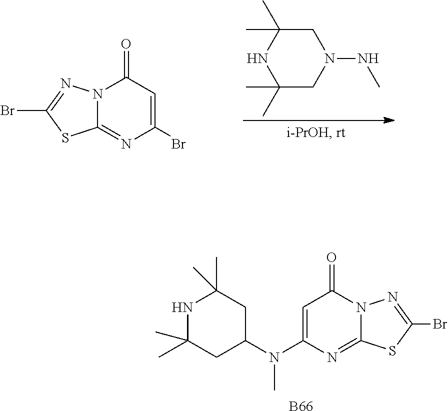

To a solution of 2,7-dibromo-5H-[1,3,4]thiadiazolo[3,2-a]pyrimidin-5-one (75 mg, 0.24 mmol) in anhydrous isopropyl alcohol (2.5 mL) was added the N,3,3,5,5-pentamethylpiperazin-1-amine (87 mg, 0.48 mmol) and the mixture was stirred at room temperature overnight. The reaction mixture was concentrated in vacuo to give a residue, the residue taken up in CH₂Cl₂ (25 mL), washed with saturated NaHCO₃ (20 mL) and water (20 mL). The organic layer was dried over Na₂SO₄, filtered, and concentrated in vacuo to give a solid. The solid was purified on a silica gel cartridge using a gradient of 0-20% methanol in CH₂Cl₂ to afford 2-bromo-7-(methyl(2,2,6,6-tetramethylpiperidin-4-yl)amino)-5H-[1,3,4]thiadiazolo[3,2-a]pyrimidin-5-one (59 mg, 61%) as a solid. LCMS (ES, m/z): 401.3 [M+H]⁺.

### Synthesis of Compound 215

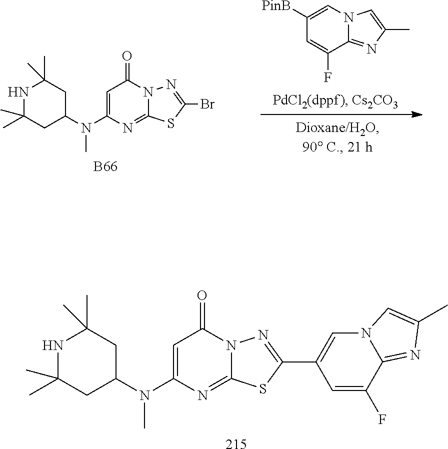

A mixture of 2-bromo-7-(methyl(2,2,6,6-tetramethylpiperidin-4-yl)amino)-5H-[1,3,4]thiadiazolo[3,2-a]pyrimidin-5-one (59 mg, 0.15 mmol), 8-fluoro-2-methyl-6-(3,3,4,4-tetramethylborolan-1-yl)imidazo[1,2-a]pyridine (46 mg, 0.16 mmol), PdCl₂(dppf) (12 mg, 0.015 mmol), and Cs₂CO₃ (97 mg, 0.29 mmol) in a mixture of dioxane (1.4 mL) and H₂O (0.14 mL) was heated at 90° C. for 21 h under nitrogen atmosphere in a sealed tube. The reaction mixture was concentrated in vacuo to give a residue, the residue taken up in CH₂Cl₂ (40 mL), washed with saturated NaHCO₃ (15 mL) and brine (20 mL). The organic phase was dried over Na₂SO₄, filtered, and concentrated in vacuo to give a residue. The residue was purified by flash chromatography on a silica gel column using a gradient of methanol from 0 to 20% in CH₂Cl₂ to afford 2-(8-fluoro-2-methylimidazo[1,2-a]pyridin-6-yl)-7-(methyl(2,2,6,6-tetramethylpiperidin-4-yl)amino)-5H-[1,3,4]thiadiazolo[3,2-a]pyrimidin-5-one (60 mg, 87%) as a solid. LCMS (ES, m z): 469.8 [M+H]⁺. ¹H NMR (CH₃OH-d₄ (0.6 mL)+CDCl₃ (0.4 mL), 400 MHz): δ 8.94 (1H, s), 7.70 (1H, s), 7.58 (1H, d, J=11.8 Hz), 6.83 (1H, s), 4.17 (1H, br, s, NH), 3.08 (3H, s), 2.43 (3H, s), 1.78 (2H, d, J=12.3 Hz), 1.56 (2H, t, J=12.3 Hz), 1.34 (6H, s), 1.22 (6H, s).

### Example 22: Synthesis of Compound 216

### Synthesis of Intermediate B67

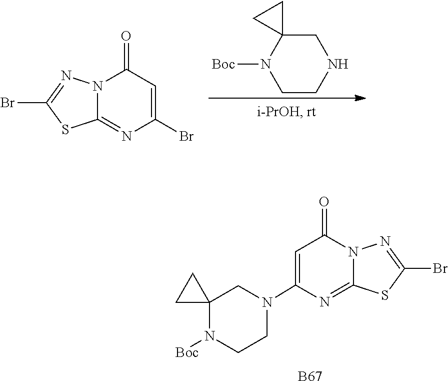 [SMILES: O=c1cc(Br)nc2sc(Br)nn12 ; CC(C)(C)OC(=O)N1CCNCC12CC2 ; "B67" ; CC(C)(C)OC(=O)N1CCN(c2cc(=O)n3nc(Br)sc3n2)CC12CC2]

To a solution of 2,7-dibromo-5H-[1,3,4]thiadiazolo[3,2-a]pyrimidin-5-one (75 mg, 0.24 mmol) in anhydrous isopropyl alcohol (2.5 mL) was added 4-Boc-4,7-diazaspiro[2.5]octane (112 mg, 0.51 mmol) and the reaction mixture was stirred for 1 h at room temperature. The reaction mixture was concentrated in vacuo to give a residue, the residue taken up in CH₂Cl₂ (25 mL), and washed with saturated NaHCO₃ (15 mL) and water (20 mL). The organic layer was dried over Na₂SO₄, filtered, and concentrated in vacuo to give a solid. The solid was purified on a silica gel cartridge using a gradient of 10-60% ethyl acetate in hexane to afford tert-butyl 7-(2-bromo-5-oxo-5H-[1,3,4]thiadiazolo[3,2-a]pyrimidin-7-yl)-4,7-diazaspiro[2.5]octane-4-carboxylate (83 mg, 78%) as a solid. LCMS (ES, m/z): 442.0, 444.0 [M+H]⁺.

### Synthesis of Intermediate B68

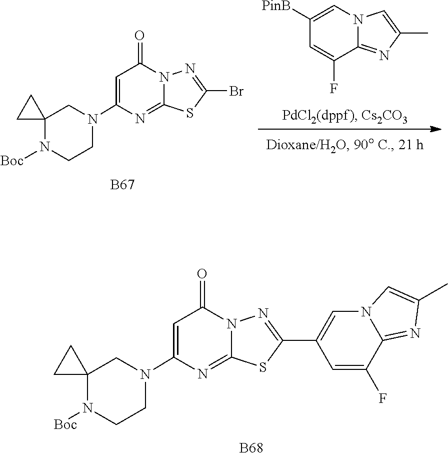

Tert-butyl 7-(2-bromo-5-oxo-5H-[1,3,4]thiadiazolo[3,2-a]pyrimidin-7-yl)-4,7-diazaspiro[2.5]octane-4-carboxylate (83 mg, 0.19 mmol), 8-fluoro-2-methyl-6-(3,3,4,4-tetramethylborolan-1-yl)imidazo[1,2-a]pyridine (58 mg, 0.21 mmol), PdCl₂(dppf)·CH₂Cl₂ (15 mg, 0.019 mmol), and Cs₂CO₃ (124 mg, 0.38 mmol) were suspended in dioxane (2 mL) and H₂O (0.2 mL) and heated at 90° C. for 21 h under nitrogen atmosphere in a sealed tube. The reaction mixture was concentrated in vacuo to give a residue, the residue taken up in CH₂Cl₂ (25 mL), and washed with saturated NaHCO₃ (15 mL) and brine (20 mL). The organic phase was dried over Na₂SO₄, filtered, and concentrated in vacuo to give a residue. The residue was purified by flash chromatography on a silica gel column using a gradient of ethyl acetate from 40 to 100% in CH₂Cl₂ to afford tert-butyl 7-(2-(8-fluoro-2-methylimidazo[1,2-a]pyridin-6-yl)-5-oxo-5H-[1,3,4]thiadiazolo[3,2-a]pyrimidin-7-yl)-4,7-diazaspiro[2.5]octane-4-carboxylate (75 mg, 78%) as a solid. LCMS (ES, m/z): 511.7 [M+H]⁺.

### Synthesis of Compound 216

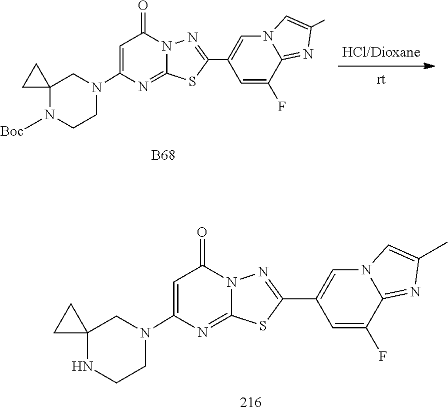 [SMILES: "B68" ; Cc1cn2cc(-c3nn4c(=O)cc(N5CCN(C(=O)OC(C)(C)C)C6(CC6)C5)nc4s3)cc(F)c2n1 ; Cc1cn2cc(-c3nn4c(=O)cc(N5CCNC6(CC6)C5)nc4s3)cc(F)c2n1 ; "216"]

A mixture of tert-butyl 7-(2-(8-fluoro-2-methylimidazo[1,2-a]pyridin-6-yl)-5-oxo-5H-[1,3,4]thiadiazolo[3,2-a]pyrimidin-7-yl)-4,7-diazaspiro[2.5]octane-4-carboxylate (75 mg, 0.15 mmol) and HCl in dioxane (4.0 M, 4.4 mL, 18 mmol), in dioxane (7 mL) was stirred at room temperature for 2 days in a sealed tube. The reaction mixture was concentrated in vacuo to give a residue, the residue taken up in CH₂Cl₂ (40 mL), and washed with saturated NaHCO₃ (15 mL). The organic layer was dried over Na₂SO₄, filtered, and concentrated in vacuo to give a solid. The solid was purified on a silica gel cartridge using a gradient of methanol from 0 to 20% in CH₂Cl₂ to afford 2-(8-fluoro-2-methylimidazo[1,2-a]pyridin-6-yl)-7-(4,7-diazaspiro[2.5]octan-7-yl)-5H-[1,3,4]thiadiazolo[3,2-a]pyrimidin-5-one (59 mg, 98%) as a solid. LCMS (ES, m/z): 411.8 [M+H]⁺. 1H NMR (CDCl₃, 400 MHz): δ 8.65 (1H, s), 7.47 (1H, s), 7.28 (1H, d, J=11.1 Hz), 6.69 (1H, s), 3.55 (2H, t, J=4.9 Hz), 3.48 (2H, s), 3.13 (2H, t, J=4.9 Hz), 2.49 (3H, s), 0.76-0.69 (4H, m).

### Example 23: Synthesis of Compound 229

### Synthesis of Intermediate B69

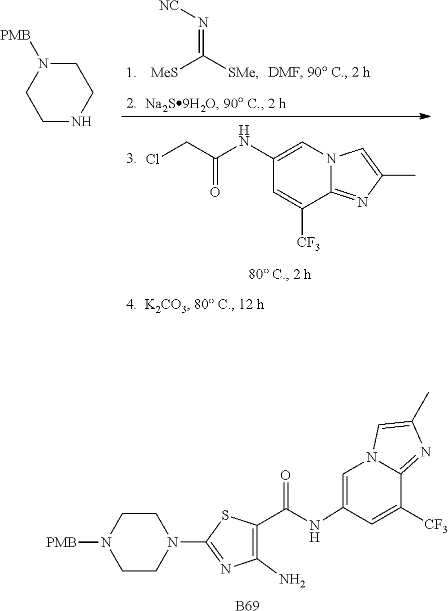

To a solution of 1-(4-methoxybenzyl)piperazine (0.10 g, 0.48 mmol) in DMF (1.5 mL) was added dimethyl-N-cyanodithioiminocarbonate (0.071 g, 0.48 mmol). The reaction mixture was heated to 90° C. for 2 h. Na₂S·9H₂O (0.11 g, 0.50 mmol) was added and the reaction mixture was stirred at 90° C. for an additional 2 h. A solution of 2-chloro-N-(2-methyl-8-(trifluoromethyl)imidazo[1,2-a]pyridin-6-yl)acetamide (0.36 g, 1.2 mmol) in DMF (1.5 mL) was added dropwise and the reaction mixture was stirred at 80° C. for 2 h. K₂CO₃ (0.067 g, 0.48 mmol) was added and the reaction mixture was stirred at 80° C. for 12 h. The reaction mixture was filtered, and concentrated in vacuo to give a residue. The residue was purified by reverse phase chromatography (C18) using 0-100% acetonitrile in water to afford 4-amino-2-(4-(4-methoxybenzyl)piperazin-1-yl)-N-(2-methyl-8-(trifluoromethyl)imidazo[1,2-a]pyridin-6-yl)thiazole-5-carboxamide (0.13 g, 40%, 80% purity) as a solid. LCMS (ES, m/z): 545.9 [M+H]⁺.

### Synthesis of Intermediate B70

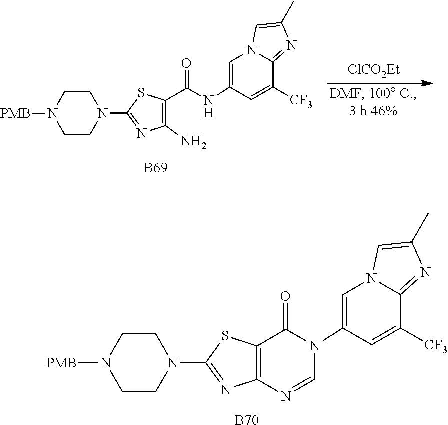

To a solution of 4-amino-2-(4-(4-methoxybenzyl)piperazin-1-yl)-N-(2-methyl-8-(trifluoromethyl)imidazo[1,2-a]pyridin-6-yl)thiazole-5-carboxamide (0.18 g, 0.33 mmol) in DMF (4.3 mL) was added ethyl chloroformate (0.63 mL, 6.6 mmol). The reaction mixture was heated to 100° C. for 3 h, then cooled to room temperature. The reaction mixture was concentrated in vacuo to give a residue. The residue was partitioned between a saturated solution of NaHCO₃ (20 mL) and DCM (20 mL), and the layers were separated. The aqueous layer was extracted with DCM (3×20 mL). The organic layers were combined, dried over Na₂SO₄, filtered, and concentrated in vacuo to give a residue. The residue was purified by column chromatography on silica gel using a gradient of 0-10% methanol in ethyl acetate to afford 2-(4-(4-methoxybenzyl)piperazin-1-yl)-6-(2-methyl-8-(trifluoromethyl)imidazo[1,2-a]pyridin-6-yl)thiazolo[4,5-d]pyrimidin-7(6H)-one (0.085 g, 46%) as a solid. LCMS (ES, m/z): 555.9 [M+H]⁺.

### Synthesis of Compound 229

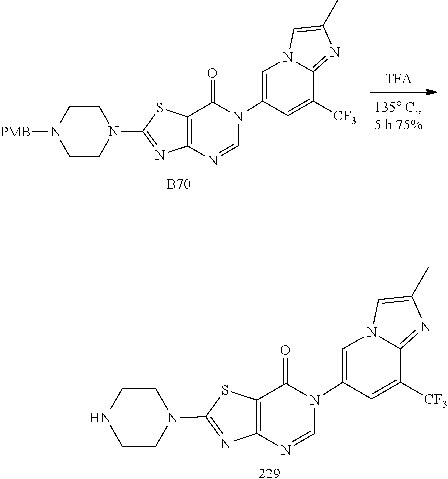

A solution of 2-(4-(4-methoxybenzyl)piperazin-1-yl)-6-(2-methyl-8-(trifluoromethyl)imidazo[1,2-a]pyridin-6-yl)thiazolo[4,5-d]pyrimidin-7(6H)-one (0.085 g, 0.15 mmol) in TFA (6.0 mL) was heated to 135° C. for 5 h. The reaction mixture was diluted with DCM (30 mL), basified with a saturated solution of NaHCO₃ to pH ˜8, and the layers were separated. The aqueous phase was extracted with DCM (3×30 mL). The organic layers were combined, dried over Na₂SO₄, filtered, and concentrated in vacuo to give a residue. The residue was purified by column chromatography on silica gel using a gradient of 0-20% methanol in DCM to afford 6-(2-methyl-8-(trifluoromethyl)imidazo[1,2-a]pyridin-6-yl)-2-(piperazin-1-yl)thiazolo[4,5-d]pyrimidin-7(6H)-one (0.050 g, 75%) as a solid. LCMS (ES, m/z): 435.7 [M+H]⁺. 1H NMR (CHCl₃-d, 400 MHz): δ_{H} 8.42 (1H, s), 8.13 (1H, s), 7.53 (2H, d, J=5.8 Hz), 3.71 (4H, s), 3.03 (4H, s), 2.56 (3H, s).

### Example 24: Synthesis of Compound 230

### Synthesis of Compound 230

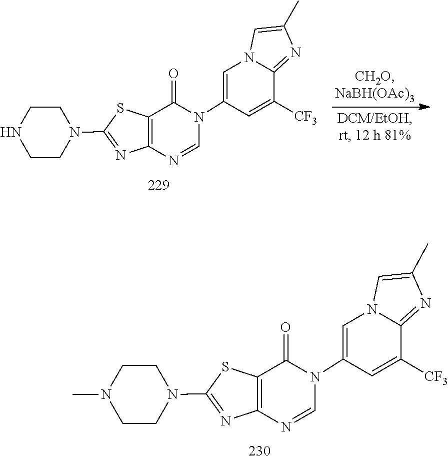

To a solution of 6-(2-methyl-8-(trifluoromethyl)imidazo[1,2-a]pyridin-6-yl)-2-(piperazin-1-yl)thiazolo[4,5-d]pyrimidin-7(6H)-one (25.0 mg, 57 μmol) in a mixture of DCM (3.0 mL) and ethanol (1.0 mL) were added formaldehyde (37% in H₂O, 29 μL, 0.28 mmol) and NaBH(OAc)₃, sequentially. The reaction mixture was stirred at room temperature for 12 h. Water (5 mL) was added, and the volatiles were evaporated under reduced pressure. Water (5 mL) and DCM (10 mL) were added, and the layers were separated. The aqueous layer was extracted with DCM (3×10 mL). The organic layers were combined, dried over Na₂SO₄, filtered, and concentrated in vacuo to give a residue. The residue was purified by column chromatography on silica gel using a gradient of 0-20% methanol in DCM to afford 6-(2-methyl-8-(trifluoromethyl)imidazo[1,2-a]pyridin-6-yl)-2-(4-methylpiperazin-1-yl)thiazolo[4,5-d]pyrimidin-7(6H)-one (21.0 mg, 81%) as a solid. LCMS (ES, m/z): 449.7 [M+H]⁺. ¹H NMR (CHCl₃-d, 400 MHz): δ_{H} 8.41 (1H, s), 8.14 (1H, s), 7.53 (2H, d, J=7.7 Hz), 3.78 (4H, s), 2.61 (3H, s), 2.56 (4H, s), 2.41 (3H, s).

### Example 25: Synthesis of Compound 231

### Synthesis of Intermediate B71

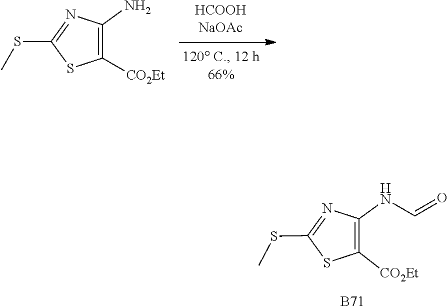

A mixture of ethyl 4-amino-2-(methylthio)thiazole-5-carboxylate (4.0 g, 18 mmol) and NaOAc (3.3 g, 41 mmol) in formic acid (30 mL, 806 mmol) was heated to 125° C. for 12 h and then cooled to room temperature. The reaction mixture was concentrated in vacuo to give a residue. The residue was partitioned between water (50 mL) and ethyl acetate (50 mL), and the layers were separated. The aqueous phase was extracted with ethyl acetate (3×50 mL). The organic layers were combined, dried over Na₂SO₄, filtered, and concentrated in vacuo to afford ethyl 4-formamido-2-(methylthio)thiazole-5-carboxylate (3.0 g, 66%) as a solid. LCMS (ES, m/z): 246.9 [M+H]⁺.

### Synthesis of Intermediate B72

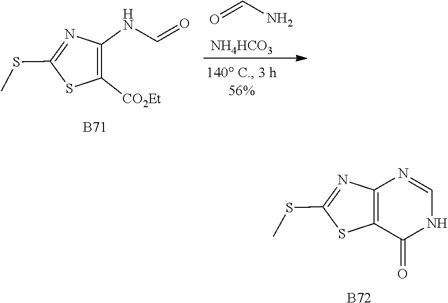

A mixture of ethyl 4-formamido-2-(methylthio)thiazole-5-carboxylate (3.0 g, 12 mmol) and NH₄HCO₃ (2.3 g, 36 mmol) in formamide (12 mL) was heated to 140° C. for 3 h, then cooled to room temperature. The reaction mixture was cooled to 0° C. to form a precipitate, and the precipitate was collected by filtration, washed with water and dried under vacuum to afford 2-(methylthio)thiazolo[4,5-d]pyrimidin-7(6H)-one (1.5 g, 56%, ˜90% purity) as a solid. LCMS (ES, m/z): 199.8 [M+H]⁺.

### Synthesis of Intermediate B73

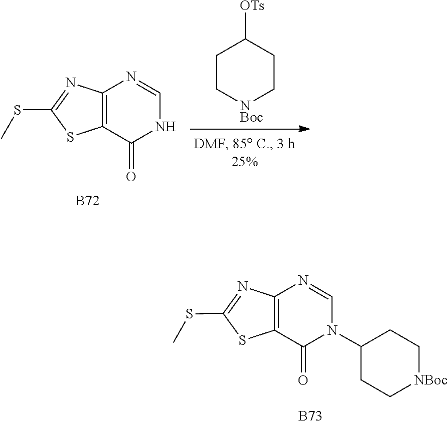 [SMILES: "B72" ; CSc1nc2nc[nH]c(=O)c2s1 ; "B73" ; CSc1nc2ncn(C3CCN(C(=O)OC(C)(C)C)CC3)c(=O)c2s1 ; Cc1ccc(S(=O)(=O)OC2CCN(C(=O)OC(C)(C)C)CC2)cc1]

A mixture of 2-(methylthio)thiazolo[4,5-d]pyrimidin-7(6H)-one 3 (1.0 g, 5.0 mmol), 1-Boc-4-(tosyloxy)piperidine (5.3 g, 15 mmol), and K₂CO₃ (1.4 g, 10 mmol) in DMF (20 mL) was heated to 85° C. for 3 h and then cooled to room temperature. The reaction mixture was concentrated in vacuo to give a residue. DCM (50 mL) was added to the residue, and the resulting mixture was filtered. The filtrate was concentrated in vacuo to give a residue. The residue was purified by column chromatography on silica gel using a gradient of 0-70% ethyl acetate in hexane to afford tert-butyl 4-(2-(methylthio)-7-oxothiazolo[4,5-d]pyrimidin-6(7H)-yl)piperidine-1-carboxylate (0.49 g, 25%) as a solid. LCMS (ES, m/z): 382.8 [M+H]⁺.

### Synthesis of Intermediate B74

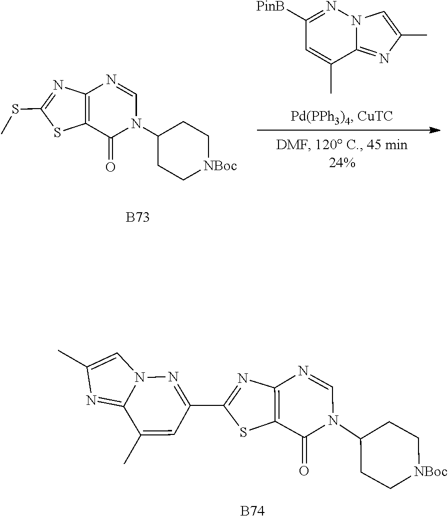

A mixture of tert-butyl 4-(2-(methylthio)-7-oxothiazolo[4,5-d]pyrimidin-6(7H)-yl)piperidine-1-carboxylate (0.15 g, 0.39 mmol), 2,8-dimethyl-6-(4,4,5,5-tetramethyl-1,3,2-dioxaborolan-2-yl)imidazo[1,2-b]pyridazine (0.43 g, 1.5 mmol), CuTC (0.22 g, 1.2 mmol), and Pd(PPh₃)₄ (0.045 g, 39 mmol) in DMF (7.5 mL) was heated to 120° C. for 45 min, then cooled to room temperature. The reaction mixture was concentrated in vacuo to give a residue. The residue was partitioned between a saturated solution of NaHCO₃ (30 mL) and DCM (30 mL), and the layers were separated. The aqueous layer was extracted with DCM (3×30 mL). The organic layers were combined, dried over Na₂SO₄, filtered, and concentrated in vacuo to give a residue. The residue was purified by column chromatography on silica gel using a gradient of 0-100% ethyl acetate and then 0-5% methanol in ethyl acetate to afford tert-butyl 4-(2-(2,8-dimethylimidazo[1,2-b]pyridazin-6-yl)-7-oxothiazolo[4,5-d]pyrimidin-6(7H)-yl)piperidine-1-carboxylate (0.045 g, 24%) as a solid. LCMS (ES, m/z): 481.9 [M+H]⁺.

Synthesis of Compound 231

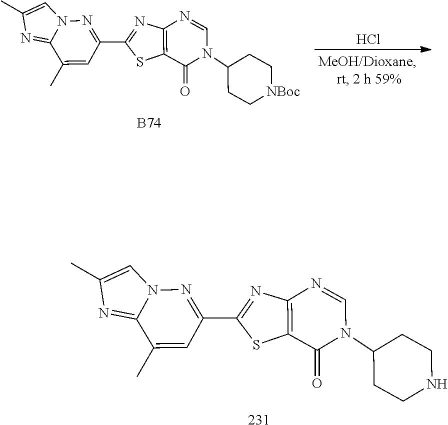 [SMILES: "B74" ; Cc1cn2nc(-c3nc4ncn(C5CCN(C(=O)OC(C)(C)C)CC5)c(=O)c4s3)cc(C)c2n1 ; Cc1cn2nc(-c3nc4ncn(C5CCNCC5)c(=O)c4s3)cc(C)c2n1 ; Cl ; "231"]

To a solution of tert-butyl 4-(2-(2,8-dimethylimidazo[1,2-b]pyridazin-6-yl)-7-oxothiazolo[4,5-d]pyrimidin-6(7H)-yl)piperidine-1-carboxylate 7 (45 mg, 0.093 mmol) in methanol (1.2 mL) was added HCl solution in dioxane (4 M, 4.8 mL, 19 mmol). The reaction mixture was stirred at room temperature for 2 h, then concentrated in vacuo to give a residue. The residue was partitioned between a saturated solution of NaHCO₃ (15 ml) and DCM (20 mL), and the layers were separated. The aqueous layer was extracted with DCM (3×20 mL). The organic layers were combined, dried over Na₂SO₄, filtered, and concentrated in vacuo to give a residue. The residue was purified by column chromatography on silica gel using a gradient of 0-10% MeOH/Et₃N (2:1 ratio) in DCM to afford 2-(2,8-dimethylimidazo[1,2-b]pyridazin-6-yl)-6-(piperidin-4-yl)thiazolo[4,5-d]pyrimidin-7(6H)-one (21 mg, 59%) as a solid. LCMS (ES, m/z): 382.2 [M+H]⁺. 1H NMR (CHCl₃-d, 400 MHz): δ_{H} 8.33 (1H, s), 7.93 (1H, s), 7.81 (1H, s), 4.97 (1H, s), 3.31 (2H, d, J=12.4 Hz), 2.87 (2H, t, J=12.0 Hz), 2.74 (3H, s), 2.56 (3H, s), 2.05 (2H, m), 1.92 (2H, m).

### Example 26: Synthesis of Compound 217

### Synthesis of Compound 217

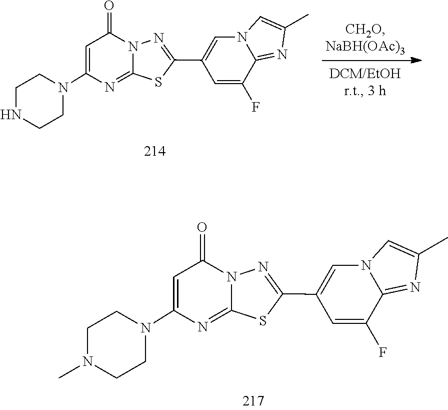

2-(8-fluoro-2-methylimidazo[1,2-a]pyridin-6-yl)-7-(piperazin-1-yl)-5H-[1,3,4]thiadiazolo[3,2-a]pyrimidin-5-one (24 mg, 0.06 mmol) was dissolved in a mixture of CH₂Cl₂ (2.5 mL) and EtOH (0.6 mL). To this solution was added formaldehyde (37% in water, 24 μL, 0.3 mmol). The reaction mixture was stirred at room temperature for 1 h. NaBH(OAc)₃ (81 mg, 0.37 mmol) was added and the reaction mixture was stirred for an additional 2 h at room temperature. The reaction mixture was concentrated in vacuo, then diluted with CH₂Cl₂ (30 mL) and washed with saturated aqueous NaHCO₃ (20 mL) and brine (20 mL). The organic layer was dried over Na₂SO₄, filtered and concentrated in vacuo to give a residue. The residue was purified by flash chromatography on a silica gel column using a gradient of 0 to 20% methanol in CH₂Cl₂ to afford 2-(8-fluoro-2-methylimidazo[1,2-a]pyridin-6-yl)-7-(4-methylpiperazin-1-yl)-5H-[1,3,4]thiadiazolo[3,2-a]pyrimidin-5-one (20 mg, 80%) as a solid. LCMS (ES, m/z): 399.8 [M+H]⁺. ¹H NMR (CH₃OH-d₄ (0.67 mL)+CDCl₃ (0.33 mL), 400 MHz): δ 8.90 (1H, s), 7.66 (1H, s), 7.53 (1H, d, J=11.7 Hz), 6.80 (1H, s), 3.64 (4H, s), 2.63 (4H, s), 2.43 (3H, s), 2.40 (3H, s).

### Example 27: Synthesis of Compound 218

### Synthesis of Compound 218

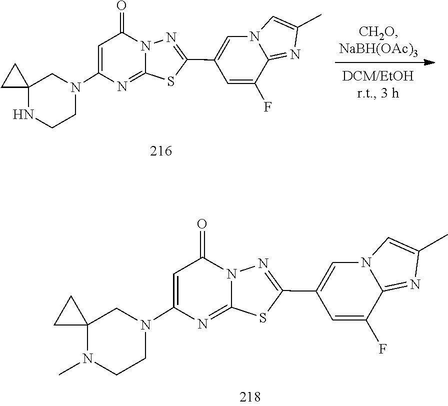 [SMILES: Cc1cn2cc(-c3nn4c(=O)cc(N5CCNC6(CC6)C5)nc4s3)cc(F)c2n1 ; Cc1cn2cc(-c3nn4c(=O)cc(N5CCN(C)C6(CC6)C5)nc4s3)cc(F)c2n1]

2-(8-fluoro-2-methylimidazo[1,2-a]pyridin-6-yl)-7-(4,7-diazaspiro[2.5]octan-7-yl)-5H-[1,3,4]thiadiazolo[3,2-a]pyrimidin-5-one (30 mg, 0.07 mmol) was dissolved in a mixture of CH₂Cl₂ (0.3 mL) and ethanol (0.07 mL). To this solution was added formaldehyde (37% in water, 27 μL, 0.36 mmol). The reaction mixture was stirred at room temperature for 2. Then NaBH(OAc)₃ (96 mg, 0.44 mmol) was added, and the reaction mixture was stirred for an additional 2 h at room temperature. The reaction mixture was concentrated in vacuo, then diluted with CH₂Cl₂ (30 mL) and washed with saturated aqueous NaHCO₃ (20 mL) and brine (20 mL). The organic layer was dried over Na₂SO₄ and the solvent was removed in vacuo to give a residue. The residue was purified by flash chromatography on a silica gel column using a gradient of 0 to 20% methanol in CH₂Cl₂ to afford 2-(8-fluoro-2-methylimidazo[1,2-a]pyridin-6-yl)-7-(4-methyl-4,7-diazaspiro[2.5]octan-7-yl)-5H-[1,3,4]thiadiazolo[3,2-a]pyrimidin-5-one (18 mg, 58%) as a solid. LCMS (ES, m/z): 425.8 [M+H]⁺. ¹H NMR (CHCl₃-d, 400 MHz): δ 8.64 (1H, s), 7.46 (1H, d, J=2.8 Hz), 7.27-7.24 (1H, m), 6.67 (1H, s), 3.55 (2H, t, J=5.2 Hz), 3.43 (2H, s), 3.08 (2H, t, J=5.1 Hz), 2.47 (2×3H, 2s), 0.80 (2H, s), 0.62 (2H, t, J=5.0 Hz).

### Example 28: Synthesis of Compound 232

### Synthesis of Intermediate B75

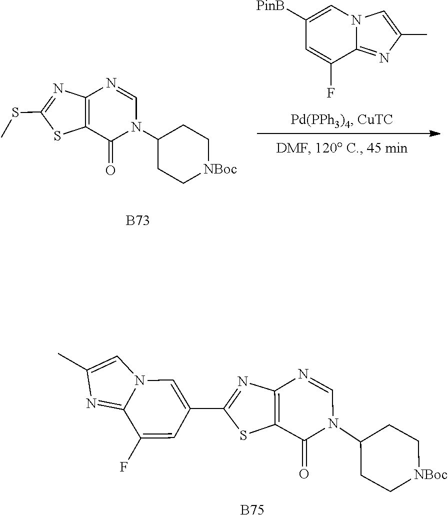

A mixture of tert-butyl 4-(2-(methylthio)-7-oxothiazolo[4,5-d]pyrimidin-6(7H)-yl)piperidine-1-carboxylate (187 mg, 0.49 mmol), 8-fluoro-2-methyl-6-(4,4,5,5-tetramethyl-1,3,2-dioxaborolan-2-yl)imidazo[1,2-a]pyridine (406 mg, 1.47 mmol), CuTC (279 mg, 1.47 mmol) and Pd(PPh₃)₄ (57 mg, 0.049 mmol) in DMF (9.0 mL) was heated to 120° C. for 45 min, then cooled to room temperature. The volatiles were evaporated under reduced pressure. A saturated solution of NaHCO₃ (30 mL) and DCM (30 mL) were added, and the layers were separated. The aqueous layer was extracted with DCM (3×30 mL). The organic layers were combined, dried over Na₂SO₄, filtered, and the filtrate concentrated under reduced pressure to give a residue. The residue was purified by column chromatography on silica gel using a gradient of 0-5% methanol in ethyl acetate to afford tert-butyl 4-(2-(8-fluoro-2-methylimidazo[1,2-a]pyridin-6-yl)-7-oxothiazolo[4,5-d]pyrimidin-6(7H)-yl)piperidine-1-carboxylate (100 mg, 42%) as a solid. LCMS (ES, m/z): 484.9 [M+H]⁺.

### Synthesis of Compound 232

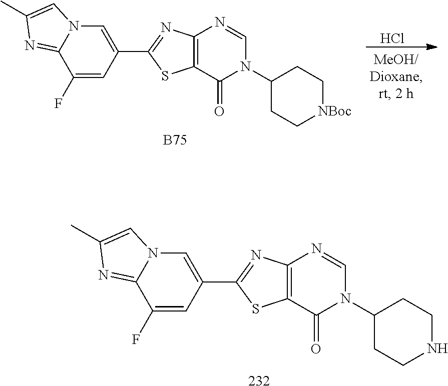 [SMILES: Cc1cn2cc(-c3nc4ncn(C5CCN(C(=O)OC(C)(C)C)CC5)c(=O)c4s3)cc(F)c2n1 ; "B75" ; Cc1cn2cc(-c3nc4ncn(C5CCNCC5)c(=O)c4s3)cc(F)c2n1 ; Cl ; "232"]

To a solution of tert-butyl 4-(2-(8-fluoro-2-methylimidazo[1,2-a]pyridin-6-yl)-7-oxothiazolo[4,5-d]pyrimidin-6(7H)-yl)piperidine-1-carboxylate (100 mg, 0.206 mmol) in methanol (3.0 mL) was added 4 M HCl solution in dioxane (4.8 mL, 19 mmol). The reaction mixture was stirred at room temperature for 2 h. The volatiles were evaporated under reduced pressure. A saturated solution of NaHCO₃ (15 mL) and DCM (20 mL) were added, and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-10% MeOH/Et₃N (2:1 ratio) in DCM to afford 2-(8-fluoro-2-methylimidazo[1,2-a]pyridin-6-yl)-6-(piperidin-4-yl)thiazolo[4,5-d]pyrimidin-7(6H)-one (21 mg, 26%) as a solid. LCMS (ES, m/z): 385.1 [M+H]⁺. 1H NMR (DMSO-d₆, 400 MHz): δ_{H} 9.41 (1H, s), 8.56 (1H, s), 7.98 (1H, s), 7.75 (1H, d, J=11.6 Hz), 4.81 (1H, s), 2.95 (2H, t, J=12.5 Hz), 2.40 (3H, s), 2.22 (2H, d, J=13.3 Hz), 2.01 (2H, m).

### Example 29: Synthesis of Compound 233

### Synthesis of Intermediate B76

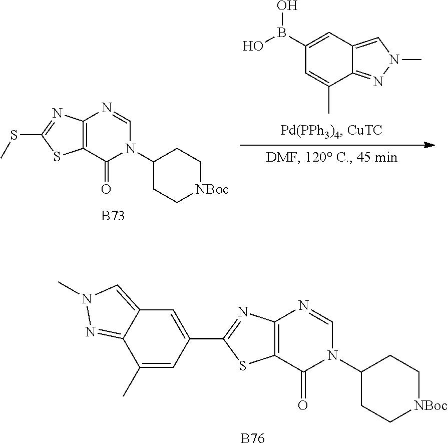 [SMILES: "B73" ; CSc1nc2ncn(C3CCN(C(=O)OC(C)(C)C)CC3)c(=O)c2s1 ; "B76" ; Cc1cc(-c2nc3ncn(C4CCN(C(=O)OC(C)(C)C)CC4)c(=O)c3s2)cc2cn(C)nc12 ; Cc1cc(B(O)O)cc2cn(C)nc12]

A mixture of tert-butyl 4-(2-(methylthio)-7-oxothiazolo[4,5-d]pyrimidin-6(7H)-yl)piperidine-1-carboxylate (100 mg, 0.26 mmol), (2,7-dimethyl-2H-indazol-5-yl)boronic acid (124 mg, 0.65 mmol), CuTC (150 mg, 0.78 mmol), and Pd(PPh₃)₄ (30 mg, 0.026 mmol) in DMF (5.0 mL) was heated to 120° C. for 45 min, then cooled to room temperature. The volatiles were evaporated under reduced pressure. A saturated solution of NaHCO₃ (30 mL) and DCM (30 mL) were added, and the layers were separated. The aqueous layer was extracted with DCM (3×30 mL). The organic layers were combined, dried over Na₂SO₄, filtered and the filtrate concentrated under reduced pressure to give a residue. The residue was purified by column chromatography on silica gel using a gradient of 0-5% methanol in ethyl acetate to afford tert-butyl 4-(2-(2,7-dimethyl-2H-indazol-5-yl)-7-oxothiazolo[4,5-d]pyrimidin-6(7H)-yl)piperidine-1-carboxylate (109 mg, 87%, 70% purity) as a solid. LCMS (ES, m/z): 480.9 [M+H]⁺.

### Synthesis of Compound 233

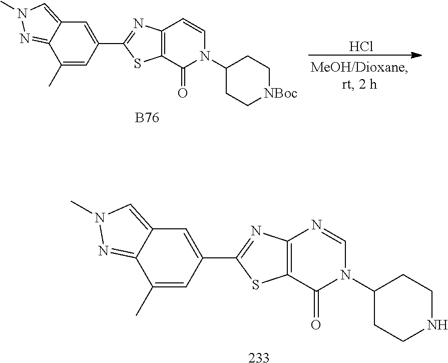 [SMILES: "B76" ; Cc1cc(-c2nc3ccn(C4CCN(C(=O)OC(C)(C)C)CC4)c(=O)c3s2)cc2cn(C)nc12 ; Cc1cc(-c2nc3ncn(C4CCNCC4)c(=O)c3s2)cc2cn(C)nc12 ; Cl ; "233"]

To a solution of tert-butyl 4-(2-(2,7-dimethyl-2H-indazol-5-yl)-7-oxothiazolo[4,5-d]pyrimidin-6(7H)-yl)piperidine-1-carboxylate (109 mg, 0.227 mmol) in methanol (3.0 mL) was added 4 M HCl solution in dioxane (4.8 mL, 19 mmol). The reaction mixture was stirred at room temperature for 2 h. The volatiles were evaporated under reduced pressure. A saturated solution of NaHCO₃ (15 ml) and DCM (20 mL) were added, and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-10% MeOH/Et₃N (2:1 ratio) in DCM to afford 2-(2,7-dimethyl-2H-indazol-5-yl)-6-(piperidin-4-yl)thiazolo[4,5-d]pyrimidin-7(6H)-one (55.0 mg, 64%) as a solid. LCMS (ES, m/z): 381.1 [M+H]⁺. ¹H NMR (DMSO-d₆, 400 MHz): δ_{H} 8.62 (1H, s), 8.56 (1H, s), 8.44 (1H, s), 7.73 (1H, s), 4.68 (1H, t, J=11.6 Hz), 4.22 (3H, s), 3.09 (2H, d, J=12.2 Hz), 2.58-2.63 (5H, m), 1.91 (2H, m), 1.80 (2H, m).

### Example 30: Synthesis of Compound 234

### Synthesis of Compound 234

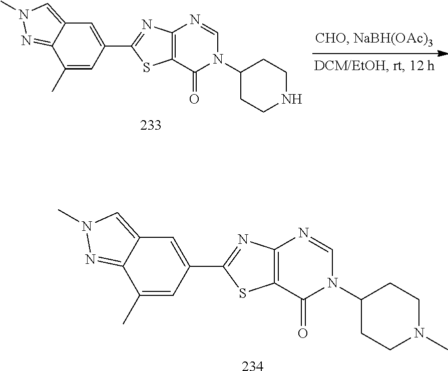

To a solution of 2-(2,7-dimethyl-2H-indazol-5-yl)-6-(piperidin-4-yl)thiazolo[4,5-d]pyrimidin-7(6H)-one (25 mg, 0.066 mmol) in a mixture of DCM (3.5 mL) and ethanol (1.0 mL) were sequentially added formaldehyde (37% in H₂O, 33 μL, 0.33 mmol) and NaBH(OAc)₃ (84 mg, 0.39 mmol). The reaction mixture was stirred at room temperature for 12 h. Water (5.0 mL) was added, and the volatiles were evaporated under reduced pressure. Water (10 mL) and DCM (30 mL) were added, and the layers were separated. The aqueous layer was extracted with DCM (3×30 mL). The organic layers were combined, dried over Na₂SO₄, filtered, and the filtrate concentrated under reduced pressure to give a residue. The residue was purified by column chromatography on silica gel using a gradient of 0-20% MeOH in DCM to afford 2-(2,7-dimethyl-2H-indazol-5-yl)-6-(1-methylpiperidin-4-yl)thiazolo[4,5-d]pyrimidin-7(6H)-one (22 mg, 85%) as a solid. LCMS (ES, m z): 395.1 [M+H]⁺. 1H NMR (DMSO-d₆, 400 MHz): δ_{H} 8.64 (1H, s), 8.54 (1H, s), 8.43 (1H, s), 7.72 (1H, s), 4.57 (1H, s), 4.21 (3H, s), 2.92 (2H, d, J=9.6 Hz), 2.57 (3H, s), 2.21 (3H, s), 2.04-2.12 (4H, m), 1.82 (2H, d, J=10.5 Hz).

### Example 31: Synthesis of Compound 235

### Synthesis of Intermediate B77

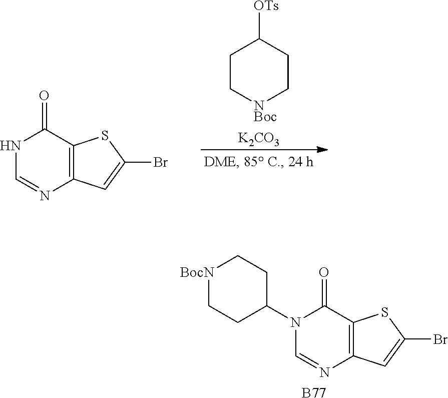

A mixture of 6-bromothieno[3,2-d]pyrimidin-4(3H)-one (2.0 g, 8.7 mmol), tert-butyl 4-(tosyloxy)piperidine-1-carboxylate (9.2 g, 26 mmol), and K₂CO₃ (2.4 g, 17 mmol) in DME (70 mL) was heated in sealed tube to 85° C. for 24 h, then cooled to room temperature. The reaction mixture was filtered, and the volatiles were evaporated under reduced pressure. A saturated solution of NaHCO₃ (100 mL) and DCM (100 mL) were added, and the layers were separated. The aqueous layer was extracted with DCM (3×50 mL). The organic layers were combined, dried over Na₂SO₄, filtered, and the filtrate concentrated under reduced pressure to give a residue. The residue was purified by column chromatography on silica gel using a gradient of 0-70% ethyl acetate in hexane to afford tert-butyl 4-(6-bromo-4-oxothieno[3,2-d]pyrimidin-3(4H)-yl)piperidine-1-carboxylate (0.89 g, 25%) as a solid. LCMS (ES, m/z): 435.7 [M+Na]⁺.

### Synthesis of Intermediate B78

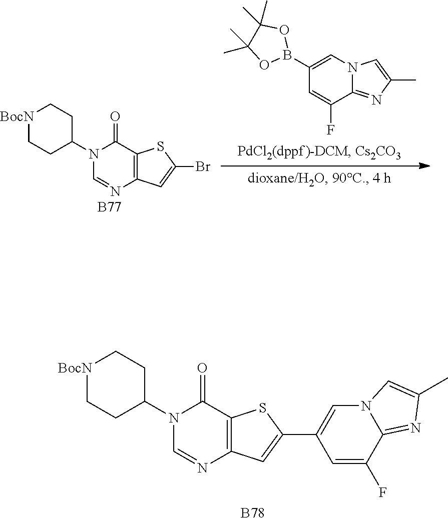

A mixture of tert-butyl 4-(6-bromo-4-oxothieno[3,2-d]pyrimidin-3(4H)-yl)piperidine-1-carboxylate (60 mg, 0.145 mmol), 8-fluoro-2-methyl-6-(4,4,5,5-tetramethyl-1,3,2-dioxaborolan-2-yl)imidazo[1,2-a]pyridine (60 mg, 0.217 mmol), Pd(dppf)Cl₂·DCM (12 mg, 0.015 mmol), and Cs₂CO₃ (94 mg, 0.29 mmol) in a mixture of dioxane (1.0 mL) and H₂O (0.05 mL) was heated to 90° C. for 4 h, then cooled to room temperature. A precipitate formed and was collected by filtration, washed with water, and dried under vacuum to afford tert-butyl 4-(6-(8-fluoro-2-methylimidazo[1,2-a]pyridin-6-yl)-4-oxothieno[3,2-d]pyrimidin-3(4H)-yl)piperidine-1-carboxylate (60 mg, 86%) as a solid. LCMS (ES, m/z): 484.0 [M+H]⁺.

### Synthesis of Compound 235

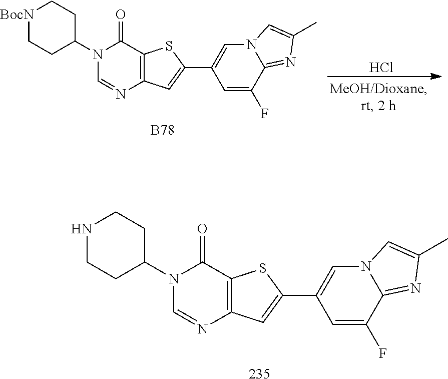 [SMILES: "B78" ; Cc1cn2cc(-c3cc4ncn(C5CCN(C(=O)OC(C)(C)C)CC5)c(=O)c4s3)cc(F)c2n1 ; Cc1cn2cc(-c3cc4ncn(C5CCNCC5)c(=O)c4s3)cc(F)c2n1 ; Cl ; "235"]

To a solution of tert-butyl 4-(6-(8-fluoro-2-methylimidazo[1,2-a]pyridin-6-yl)-4-oxothieno[3,2-d]pyrimidin-3(4H)-yl)piperidine-1-carboxylate (60 mg, 0.124 mmol) in methanol (1.6 mL) was added 4 M HCl solution in dioxane (2.7 mL, 10.8 mmol). The reaction mixture was stirred at room temperature for 2 h. The volatiles were evaporated under reduced pressure. An aqueous solution of NaOH (0.1 M, 20 mL) and DCM (40 mL) were added, and the layers were separated. The aqueous layer was extracted with DCM (3×20 mL). Following this, the aqueous layer was extracted with CHCl₃/iPrOH (3×20 mL, 9:1). The organic layers were combined, dried over Na₂SO₄, filtered, and the filtrate concentrated under reduced pressure. Water (20 mL) was added, and the mixture was sonicated. The suspended white solid was filtered, washed with water (10 mL), and dried under vacuum to afford 6-(8-fluoro-2-methylimidazo[1,2-a]pyridin-6-yl)-3-(piperidin-4-yl)thieno[3,2-d]pyrimidin-4(3H)-one (43 mg, 90%) as a solid. LCMS (ES, m/z): 384.1 [M+H]⁺. 1H NMR (DMSO-d₆, 400 MHz): δ_{H} 9.02 (1H, s), 8.51 (1H, s), 7.89 (1H, s), 7.86 (1H, s), 7.72 (1H, d, J=12.1 Hz), 4.70 (1H, br s), 3.10 (2H, d, J=12.4 Hz), 2.62 (2H, m), 2.38 (3H, s), 1.86 (4H, m).

### Example 32: Synthesis of Compound 236

### Synthesis of Intermediate B79

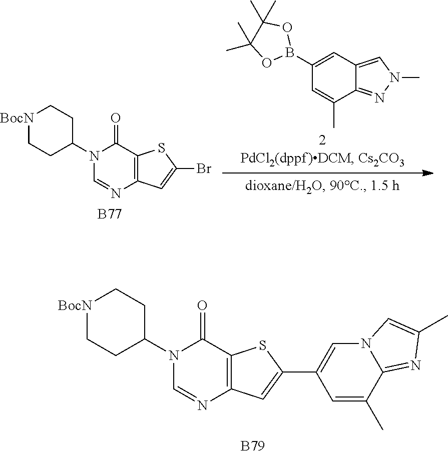

A mixture of tert-butyl 4-(6-bromo-4-oxothieno[3,2-d]pyrimidin-3(4H)-yl)piperidine-1-carboxylate (200 mg, 0.48 mmol), 2,7-dimethyl-5-(4,4,5,5-tetramethyl-1,3,2-dioxaborolan-2-yl)-2H-indazole (197 mg, 0.724 mmol), Cs₂CO₃ (316 mg, 0.97 mmol), and Pd(dppf)Cl₂·DCM (39 mg, 0.048 mmol) in a mixture of dioxane (3.5 mL) and H₂O (0.20 mL) was heated to 90° C. for 1.5 h, then cooled to room temperature. The reaction mixture was filtered, and volatiles were evaporated. Water (20 mL) and DCM (20 mL) were added, and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-10% methanol in ethyl acetate to afford tert-butyl 4-(6-(2,7-dimethyl-2H-indazol-5-yl)-4-oxothieno[3,2-d]pyrimidin-3(4H)-yl)piperidine-1-carboxylate (200 mg, 86%, 70% purity) as a solid. LCMS (ES, m/z): 479.9 [M+H]⁺.

### Synthesis of Compound 236

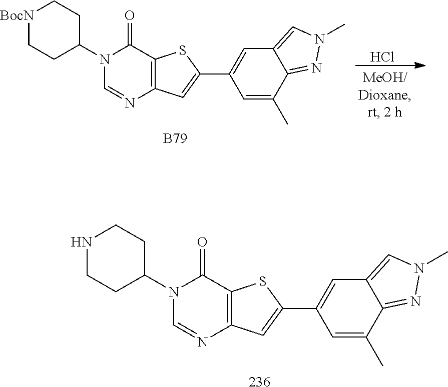 [SMILES: "B79" ; Cc1cc(-c2cc3ncn(C4CCN(C(=O)OC(C)(C)C)CC4)c(=O)c3s2)cc2cn(C)nc12 ; Cc1cc(-c2cc3ncn(C4CCNCC4)c(=O)c3s2)cc2cn(C)nc12 ; Cl ; "236"]

To a solution of tert-butyl 4-(6-(2,7-dimethyl-2H-indazol-5-yl)-4-oxothieno[3,2-d]pyrimidin-3(4H)-yl)piperidine-1-carboxylate (190 mg, 0.396 mmol) in methanol (5.0 mL) was added 4 M HCl solution in dioxane (8.5 mL, 34 mmol). The reaction mixture was stirred at room temperature for 2 h. The volatiles were evaporated under reduced pressure. An aqueous solution of NaHCO₃ (20 mL) and DCM (30 mL) were added, and the layers were separated. The aqueous layer was extracted with DCM (3×30 mL). The organic layers were combined, dried over Na₂SO₄, filtered, and concentrated under reduced pressure to give a residue. The residue was purified by column chromatography on silica gel using a gradient of 0-15% MeOH:Et₃N (2:1 ratio) in DCM. The fractions containing product were collected and concentrated under reduced pressure. Water (10 mL) and DCM (10 mL) were added, and the layers were separated. The aqueous phase was extracted with DCM (2×10 mL). The organic layers were combined, dried over Na₂SO₄, filtered, and concentrated under reduced pressure to afford 6-(2,7-dimethyl-2H-indazol-5-yl)-3-(piperidin-4-yl)thieno[3,2-d]pyrimidin-4(3H)-one (105 mg, 70%) as a solid. LCMS (ES, m/z): 380.1 [M+H]⁺. 1H NMR (DMSO-d₆, 400 MHz): δ_{H} 8.47 (1H, s), 8.43 (1H, s), 8.05 (1H, s), 7.76 (1H, s), 7.52 (1H, s), 4.67-4.73 (1H, m), 4.20 (3H, s), 3.08 (2H, d, J=12.2 Hz), 2.56-2.63 (5H, m), 1.86-1.94 (2H, m), 1.78 (2H, d, J=11.4 Hz).

### Example 33: Synthesis of Compound 237

### Synthesis of Compound 237

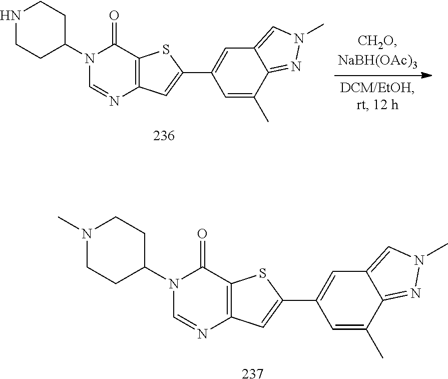

To a solution of 6-(2,7-dimethyl-2H-indazol-5-yl)-3-(piperidin-4-yl)thieno[3,2-d]pyrimidin-4(3H)-one (30 mg, 0.079 mmol) in a mixture of DCM (3.5 mL) and ethanol (1.0 mL) were sequentially added formaldehyde (37% in H₂O, 39 μL, 0.39 mmol) and NaBH(OAc)₃ (101 mg, 0.47 mmol). The reaction mixture was stirred at room temperature for 12 h. Water (5.0 mL) was added, and the volatiles were evaporated under reduced pressure. Water (10 mL) and DCM (30 mL) were added, and the layers were separated. The aqueous layer was extracted with DCM (3×30 mL). The organic layers were combined, dried over Na₂SO₄, filtered, and the filtrate concentrated under reduced pressure to give a residue. The residue was purified by column chromatography on silica gel using a gradient of 0-10% MeOH/Et₃N (2:1) in DCM. The fractions containing products were collected and concentrated under reduced pressure. Water (10 mL) and DCM (10 mL) were added, and the layers were separated. The aqueous layer was extracted with DCM (2×10 mL). The organic layers were combined, dried over Na₂SO₄, filtered, and concentrated under reduced pressure to afford 6-(2,7-dimethyl-2H-indazol-5-yl)-3-(1-methylpiperidin-4-yl)thieno[3,2-d]pyrimidin-4(3H)-one (26 mg, 84%) as a solid. LCMS (ES, m z): 394.2 [M+H]⁺. 1H NMR (DMSO-d₆, 400 MHz): δ_{H} 8.51 (1H, s), 8.43 (1H, s), 8.06 (1H, s), 7.77 (1H, s), 7.53 (1H, s), 4.60 (1H, m), 4.20 (3H, s), 2.92 (2H, d, J=9.5 Hz), 2.56 (3H, s), 2.22 (3H, s), 2.01-2.15 (4H, m), 1.81 (2H, d, J=10.8 Hz).

### Example 34: Synthesis of Compound 238

### Synthesis of Intermediate B80

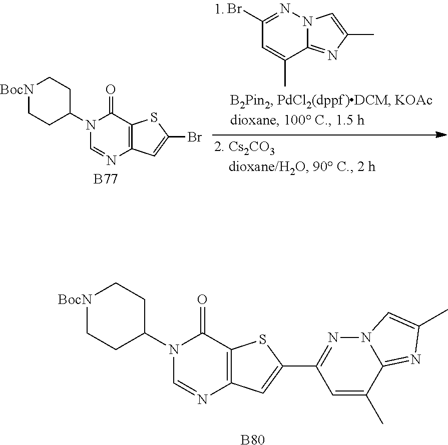 [SMILES: "B77" ; "B80" ; CC(C)(C)OC(=O)N1CCC(n2cnc3cc(Br)sc3c2=O)CC1 ; Cc1cn2nc(-c3cc4ncn(C5CCN(C(=O)OC(C)(C)C)CC5)c(=O)c4s3)cc(C)c2n1 ; Cc1cn2nc(Br)cc(C)c2n1]

A mixture of 6-bromo-2,8-dimethylimidazo[1,2-b]pyridazine (200 mg, 0.885 mmol), Bis(pinacolato)diboron (246 mg, 0.97 mmol), PdCl₂(dppf).DCM (65 mg, 0.088 mmol), and KOAc (255 mg, 2.6 mmol) in dioxane (3.0 mL) was heated to 100° C. for 1.5 h, then cooled to room temperature. A solution of tert-butyl 4-(6-bromo-4-oxothieno[3,2-d]pyrimidin-3(4H)-yl)piperidine-1-carboxylate (202 mg, 0.49 mmol) in dioxane (2.0 mL), Cs₂CO₃ (865 mg, 2.6 mmol) and H₂O (0.50 mL) were sequentially added. The reaction mixture was heated at 90° C. for 2 h, then cooled to room temperature. The reaction mixture was filtered over Celite using 10% MeOH in DCM as eluent. The volatiles were evaporated under reduced pressure. Water (20 mL) and DCM (20 mL) were added, and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-15% methanol in ethyl acetate to afford tert-butyl 4-(6-(2,8-dimethylimidazo[1,2-b]pyridazin-6-yl)-4-oxothieno[3,2-d]pyrimidin-3(4H)-yl)piperidine-1-carboxylate (173 mg, 74%) as a solid. LCMS (ES, m/z): 480.9 [M+H]⁺.

### Synthesis of Compound 238

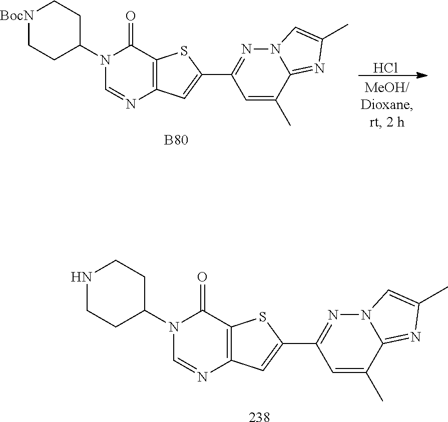 [SMILES: "B80" ; Cc1cn2nc(-c3cc4ncn(C5CCN(C(=O)OC(C)(C)C)CC5)c(=O)c4s3)cc(C)c2n1 ; Cc1cn2nc(-c3cc4ncn(C5CCNCC5)c(=O)c4s3)cc(C)c2n1 ; Cl ; "238"]

To a solution of tert-butyl 4-(6-(2,8-dimethylimidazo[1,2-b]pyridazin-6-yl)-4-oxothieno[3,2-d]pyrimidin-3(4H)-yl)piperidine-1-carboxylate (172 mg, 0.36 mmol) in methanol (5.0 mL) was added 4 M HCl solution in dioxane (7.7 mL, 31 mmol). The reaction mixture was stirred at room temperature for 2 h. The volatiles were evaporated under reduced pressure. A saturated solution of NaHCO₃ (20 mL) and DCM (30 mL) were added, and the layers were separated. The aqueous layer was extracted with DCM (3×30 mL). The organic layers were combined, dried over Na₂SO₄, filtered, and the filtrate concentrated under reduced pressure to give a residue. The residue was purified by column chromatography on silica gel using a gradient of 0-15% MeOH:Et₃N (2:1 ratio) in DCM. The fractions containing product were collected and concentrated under reduced pressure. Water (10 mL) and DCM were added, and the layers were separated. The aqueous phase was extracted with DCM (2×10 mL). The organic layers were combined, dried over Na₂SO₄, filtered, and the filtrated concentrated under reduced pressure to afford 6-(2,8-dimethylimidazo[1,2-b]pyridazin-6-yl)-3-(piperidin-4-yl)thieno[3,2-d]pyrimidin-4(3H)-one (105 mg, 77%) as a solid. LCMS (ES, m/z): 381.1 [M+H]⁺. ¹H NMR (DMSO-d₆, 400 MHz): δ_{H} 8.52 (1H, s), 8.21 (1H, s), 8.10 (1H, s), 7.89 (1H, s), 4.70 (1H, t, J=11.7 Hz), 3.10 (2H, d, J=12.3 Hz), 2.64 (2H, m), 2.60 (3H, s), 2.40 (3H, s), 1.93 (2H, m), 1.80 (2H, d, J=11.4 Hz).

### Example 35: Synthesis of Compound 239

### Synthesis of Compound 239

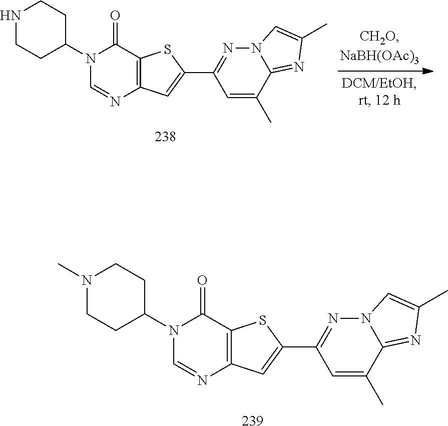

To a solution of 6-(2,8-dimethylimidazo[1,2-b]pyridazin-6-yl)-3-(piperidin-4-yl)thieno[3,2-d]pyrimidin-4(3H)-one (35 mg, 0.092 mmol) in a mixture of DCM (3.5 mL) and ethanol (1.0 mL) were sequentially added formaldehyde (37% in H₂O, 46 μL, 0.46 mmol) and NaBH(OAc)₃ (117 mg, 0.55 mmol). The reaction mixture was stirred at room temperature for 12 h. Water (5.0 mL) was added, and the volatile were evaporated under reduced pressure. Water (10 mL) and DCM (30 mL) were added, and the layers were separated. The aqueous layer was extracted with DCM (3×30 mL). The organic layers were combined, dried over Na₂SO₄, filtered, and the filtrate concentrated under reduced pressure to give a residue. The residue was purified by column chromatography on silica gel using a gradient of 0-10% MeOH/Et₃N (2:1) in DCM. The fractions containing product were collected and concentrated under reduced pressure. Water (5.0 mL) and DCM (10 mL) were added, and the layers were separated. The aqueous layer was extracted with DCM (3×10 mL). The organic layers were combined, dried over Na₂SO₄, filtered and the filtrate concentrated under reduced pressure to afford 6-(2,8-dimethylimidazo[1,2-b]pyridazin-6-yl)-3-(1-methylpiperidin-4-yl)thieno[3,2-d]pyrimidin-4(3H)-one (24 mg, 66%) as a solid. LCMS (ES, m/z): 395.1 [M+H]⁺. ¹H NMR (DMSO-d₆, 400 MHz): δ_{H} 8.56 (1H, s), 8.21 (1H, s), 8.10 (1H, s), 7.89 (1H, s), 4.60 (1H, m), 2.92 (2H, d, J=9.8 Hz), 2.60 (3H, s), 2.40 (3H, s), 2.22 (3H, s), 2.02-2.16 (4H, m), 1.82 (2H, d, J=10.8 Hz).

### Example 36: Synthesis of Compound 252

### Synthesis of Intermediate B81

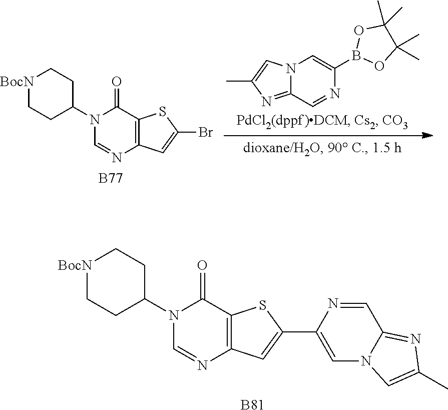

A mixture of tert-butyl 4-(6-bromo-4-oxothieno[3,2-d]pyrimidin-3(4H)-yl)piperidine-1-carboxylate (150 mg, 0.36 mmol), 2-methyl-6-(4,4,5,5-tetramethyl-1,3,2-dioxaborolan-2-yl)imidazo[1,2-a]pyrazine (141 mg, 0.54 mmol), Cs₂CO₃ (354 mg, 1.1 mmol), and Pd(dppf)Cl₂·DCM (30 mg, 0.036 mmol) in a mixture of dioxane (4.0 mL) and water (0.7 mL) was heated to 90° C. for 1.5 h, then cooled to room temperature. The reaction mixture was filtered over Celite using 10% methanol in DCM as eluent. The volatiles were evaporated under reduced pressure. Water (15 mL) and DCM (15 mL) were added, and the layers were separated. The aqueous layer was extracted with DCM (3×15 mL). The organic layers were combined, dried over Na₂SO₄, filtered, and the filtrate concentrated under reduced pressure to give a residue. The residue was purified by column chromatography on silica gel using a gradient of 0-15% methanol in ethyl acetate to afford tert-butyl 4-(6-(2-methylimidazo[1,2-a]pyrazin-6-yl)-4-oxothieno[3,2-d]pyrimidin-3(4H)-yl)piperidine-1-carboxylate (100 mg, 59%) as a solid.

LCMS (ES, m/z): 467.2 [M+H]⁺.

Synthesis of Compound 252

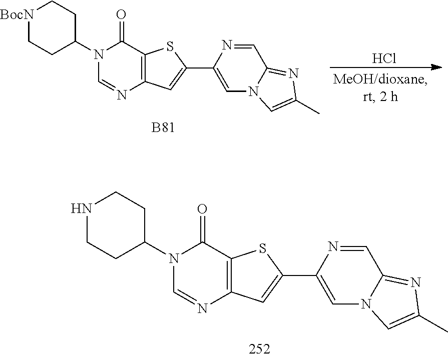 [SMILES: "B81" ; Cc1cn2cc(-c3cc4ncn(C5CCN(C(=O)OC(C)(C)C)CC5)c(=O)c4s3)ncc2n1 ; Cc1cn2cc(-c3cc4ncn(C5CCNCC5)c(=O)c4s3)ncc2n1 ; Cl ; "252"]

To a solution of tert-butyl 4-(6-(2-methylimidazo[1,2-a]pyrazin-6-yl)-4-oxothieno[3,2-d]pyrimidin-3(4H)-yl)piperidine-1-carboxylate (100 mg, 0.215 mmol) in methanol (10 mL) was added 4 M HCl solution in dioxane (5.0 mL, 20 mmol). The reaction mixture was stirred at room temperature for 2 h. The volatiles were evaporated under reduced pressure. An aqueous solution of NaHCO₃ (20 mL) and DCM (30 mL) were added, and the layers were separated. The aqueous layer was extracted with DCM (3×30 mL). The aqueous layer was then extracted with 15% iPrOH in CHCl₃ (3×30 mL). The organic layers were combined, dried over Na₂SO₄, filtered, and the filtrate concentrated under reduced pressure. MeCN (5 mL) was added, and the mixture was sonicated. A suspended solid was collected by filtration, washed with MeCN (10 mL), and dried under vacuum to afford 6-(2-methylimidazo[1,2-a]pyrazin-6-yl)-3-(piperidin-4-yl)thieno[3,2-d]pyrimidin-4(3H)-one (40 mg, 51%) as a solid. LCMS (ES, m/z): 367.1 [M+H]⁺. 1H NMR (DMSO-d₆, 400 MHz): δ_{H} 9.43 (1H, s), 9.01 (1H, s), 8.49 (1H, s), 7.94 (1H, s), 7.93 (1H, s), 4.69 (1H, m), 3.09 (2H, d, J=12.2 Hz), 2.61 (2H, t, J=12.0 Hz), 2.45 (3H, s), 1.90 (2H, m), 1.79 (2H, d, J=11.3 Hz).

### Example 37: Synthesis of Compound 253

### Synthesis of Intermediate B82

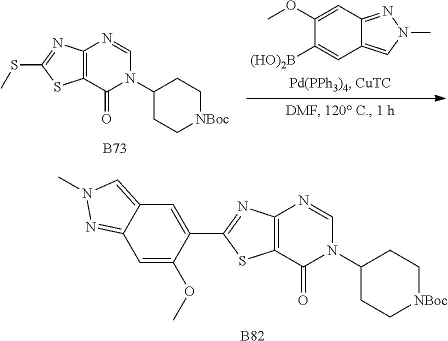

A mixture of tert-butyl 4-(2-(methylthio)-7-oxothiazolo[4,5-d]pyrimidin-6(7H)-yl)piperidine-1-carboxylate (65 mg, 0.17 mmol), (6-methoxy-2-methyl-2H-indazol-5-yl)boronic acid (70 mg, 0.34 mmol), CuTC (97 mg, 0.51 mmol), and Pd(PPh₃)₄ (20 mg, 0.017 mmol) in DMF (3.5 mL) was heated to 120° C. for 1 h, then cooled to room temperature. The volatiles were evaporated under reduced pressure. A saturated solution of NaHCO₃ (20 mL) and DCM (20 mL) were added, and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-10% methanol in ethyl acetate to afford tert-butyl 4-(2-(6-methoxy-2-methyl-2H-indazol-5-yl)-7-oxothiazolo[4,5-d]pyrimidin-6(7H)-yl)piperidine-1-carboxylate (46 mg, 55%) as a solid. LCMS (ES, m/z): 497.2 [M+H]⁺.

### Synthesis of Compound 253

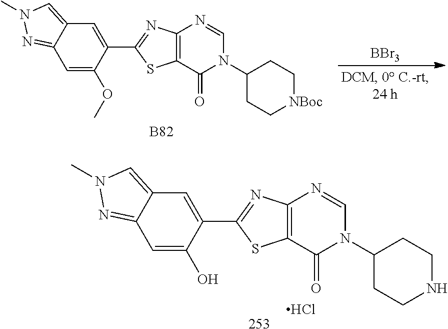 [SMILES: "B82" ; COc1cc2nn(C)cc2cc1-c1nc2ncn(C3CCN(C(=O)OC(C)(C)C)CC3)c(=O)c2s1 ; Cn1cc2cc(-c3nc4ncn(C5CCNCC5)c(=O)c4s3)c(O)cc2n1 ; "253" ; Cl ; "BBr3"]

To a solution of tert-butyl 4-(2-(6-methoxy-2-methyl-2H-indazol-5-yl)-7-oxothiazolo[4,5-d]pyrimidin-6(7H)-yl)piperidine-1-carboxylate (46 mg, 0.093 mmol) in DCM (4.0 mL) cooled to 0° C. was added dropwise boron tribromide (0.22 mL, 2.3 mmol) under argon. The reaction mixture was allowed to warm to room temperature and stirred for 24 h. The mixture was cooled to 0° C. and cold MeOH (5.0 mL) was added dropwise. The volatiles were evaporated under reduced pressure. A saturated solution of NaHCO₃ (15 mL) and DCM (15 mL) were added, and the layers were separated. The aqueous layer was extracted with DCM (3×15 mL). The organic layers were combined, dried over Na₂SO₄, filtered, and the filtrate concentrated under reduced pressure to give a residue. The residue was purified by column chromatography on silica gel using a gradient of 0-20% MeOH:Et₃N (2:1 ratio) in DCM. The fractions containing product were collected and evaporated under reduced pressure to give a residue. The residue was then purified by reverse phase chromatography (C18) using a gradient of 0-40% MeCN in water (containing 0.1% HCl) to afford 2-(6-hydroxy-2-methyl-2H-indazol-5-yl)-6-(piperidin-4-yl)thiazolo[4,5-d]pyrimidin-7(6H)-one, hydrochloride (4.5 mg, 12%) as an HCl salt. LCMS (ES, m/z): 383.1 [M+H]⁺. ¹H NMR (H₂O-d₂, 400 MHz): δ_{H} 8.35 (1H, s), 8.25 (1H, s), 8.14 (1H, s), 6.64 (1H, s), 4.86-4.80 (m, 1H), 4.05 (3H, s), 3.75 (2H, d, J=13.0 Hz), 3.33 (2H, t, J=12.7 Hz), 2.35-2.41 (4H, m).

### Example 38: Synthesis of Compound 254

### Synthesis of Intermediate B83

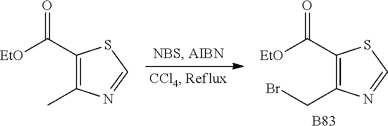

To a solution of ethyl 4-methylthiazole-5-carboxylate (3.50 g, 19.4 mmol) dissolved in carbon tetrachloride (97.1 mL) was added N-bromosuccinimide (3.67 g, 20.4 mmol) and 2,2′-azobis(2-methylpropionitrile) (159 mg, 971 umol). The reaction mixture was stirred at room temperature for 30 minutes, then under reflux overnight. The reaction mixture was washed with 1 M Na₂S₂O₃ (50 mL), 0.5 M NaOH (50 mL), and brine (2×50 mL). The organic phase was dried over Na₂SO₄, filtered, and concentrated in vacuo to give a residue. The residue was purified by flash chromatography on silica gel using a gradient of 0-50% ethyl acetate in hexane to afford ethyl 4-(bromomethyl)thiazole-5-carboxylate (3.57 g, 73%) as an oil. LCMS (ES, m/z): 249.9 [M+H]⁺.

### Synthesis of Intermediate B84

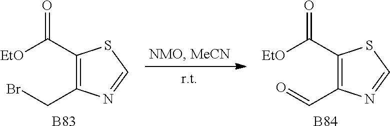 [SMILES: "B83" ; CCOC(=O)c1scnc1CBr ; "B84" ; CCOC(=O)c1scnc1C=O]

To a solution of ethyl 4-(bromomethyl)thiazole-5-carboxylate 2 (3.6 g, 14 mmol) dissolved in MeCN (143 mL) was added N-methylmorpholine-N-oxide (8.4 g, 71 mmol). The reaction mixture was stirred at room temperature for 3 hours, then diluted with ethyl acetate (200 mL) and washed with water (200 mL) and brine (200 mL). The organic phase was dried over Na₂SO₄, filtered, and the filtrate concentrated under reduced pressure to give a residue. The residue was purified by flash chromatography on silica gel using a gradient of 0-50% ethyl acetate in hexane to afford ethyl 4-formylthiazole-5-carboxylate (1.8 g, 68%) as a solid. LCMS (ES, m/z): 186.1 [M+H]⁺.

### Synthesis of Intermediate B85

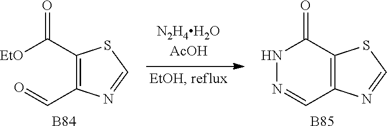 [SMILES: "B84" ; CCOC(=O)c1scnc1C=O ; "B85" ; O=c1[nH]ncc2ncsc12]

To a solution of ethyl 4-formylthiazole-5-carboxylate (1.8 g, 9.72 mmol) dissolved in ethanol (49 mL) was added acetic acid (2.0 mL). The reaction mixture was heated under reflux for 48 hours, then evaporated to dryness under reduced pressure to afford thiazolo[4,5-d]pyridazin-7(6H)-one (1.4 g, 94%) as a solid. LCMS (ES, m/z): 154.0 [M+H]⁺.

### Synthesis of Intermediate B86

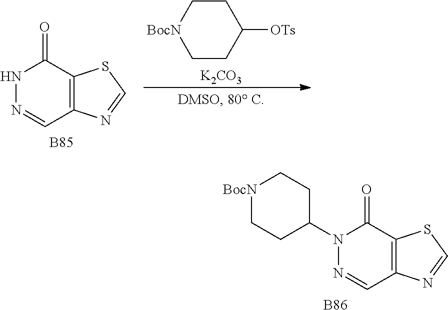

To a mixture of thiazolo[4,5-d]pyridazin-7(6H)-one 4 (1.1 g, 6.9 mmol) and tert-butyl 4-(tosyloxy)piperidine-1-carboxylate (7.4 g, 21 mmol) was added DMSO (69 mL), followed by K₂CO₃ (1.4 g, 10 mmol). The reaction mixture was heated at 80° C. overnight, then diluted with ethyl acetate (200 mL) and washed with saturated NH₄Cl (100 mL), NaHCO₃ (100 mL), and brine (2×100 mL). The organic phase was dried over Na₂SO₄ and concentrated in vacuo to give a residue. The residue was purified by flash chromatography on silica gel using a gradient of 0-50% ethyl acetate in hexane to afford tert-butyl 4-(7-oxothiazolo[4,5-d]pyridazin-6(7H)-yl)piperidine-1-carboxylate (693 mg, 30%) as a solid. LCMS (ES, m/z): 281.1 [M-^{t}Bu]⁺.

### Synthesis of Intermediate B87

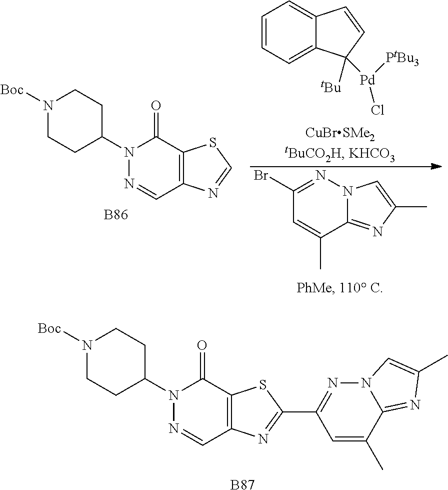

A flame-dried sealed tube was charged with tert-butyl 4-(7-oxothiazolo[4,5-d]pyridazin-6(7H)-yl)piperidine-1-carboxylate (82 mg, 0.24 mmol), 6-bromo-2,8-dimethylimidazo[1,2-b]pyridazine (50 mg, 0.22 mmol), pivalic acid (9.0 mg, 89 μmol), potassium bicarbonate (45 mg, 0.44 mmol), and CuBr SMe₂ (9.1 mg, 44 μmol). The mixture was dissolved in dry toluene (2.2 mL) and argon was bubbled through the mixture for 10 minutes. To the reaction mixture was added chloro(1-tert-butyl-1H-inden-1-yl)(tri-tert-butylphosphine)palladium(II) (5.7 mg, 11 μmol) under flow of argon. The tube was sealed, and the reaction mixture was heated at 110° C. overnight. The reaction mixture was diluted with ethyl acetate (25 mL) and filtered through a pad of celite using additional ethyl acetate (10 mL) to wash the filter cake. The filtrate was washed with saturated NaHCO₃ (20 mL) and brine (2×20 mL). The organic phase was dried over Na₂SO₄, filtered, and concentrated in vacuo to give a residue. The residue was purified by flash chromatography on a silica gel column using a gradient of 60-100% ethyl acetate in hexane to afford tert-butyl 4-(2-(2,8-dimethylimidazo[1,2-b]pyridazin-6-yl)-7-oxothiazolo[4,5-d]pyridazin-6(7H)-yl)piperidine-1-carboxylate (21 mg, 20%) as a solid. LCMS (ES, m/z): 482.2 [M+H]⁺.

### Synthesis of Compound 254

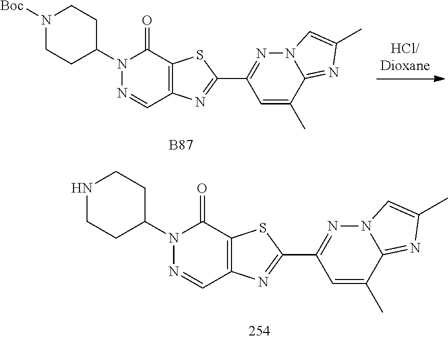 [SMILES: "B87" ; Cc1cn2nc(-c3nc4cnn(C5CCN(C(=O)OC(C)(C)C)CC5)c(=O)c4s3)cc(C)c2n1 ; Cc1cn2nc(-c3nc4cnn(C5CCNCC5)c(=O)c4s3)cc(C)c2n1]

To tert-butyl 4-(2-(2,8-dimethylimidazo[1,2-b]pyridazin-6-yl)-7-oxothiazolo[4,5-d]pyridazin-6(7H)-yl)piperidine-1-carboxylate (21 mg, 0.42 mmol) was added HCl 4.0 M in dioxane (1.5 mL). The reaction mixture was stirred vigorously at room temperature for 90 minutes, then concentrated to dryness under reduced pressure. The residue was partitioned between DCM (20 mL) and 0.25 M NaOH (20 mL) and stirred to neutralize. The phases were separated, and the aqueous phase was washed with DCM (2×20 mL). The organic phases were combined, dried over Na₂SO₄, filtered, and the filtrate concentrated in vacuo. The residue was purified by flash chromatography on a neutral alumina column using a gradient of 0-10% MeOH in DCM to afford 2-(2,8-dimethylimidazo[1,2-b]pyridazin-6-yl)-6-(piperidin-4-yl)thiazolo[4,5-d]pyridazin-7(6H)-one (5.6 mg, 35%) as a solid. LCMS (ES, m/z): 382.1 [M+H]⁺. ¹H NMR (CDCl₃, 300 MHz): δ 8.58 (1H, s), 7.85 (2H, s), 5.14-5.23 (1H, m), 3.29 (2H, d, J=13.3 Hz), 2.87 (2H, t, J=11.9 Hz), 2.78 (3H, s), 2.59 (3H, s), 1.89-2.12 (4H, m).

### Example 39: Synthesis of Compound 255

### Synthesis of Intermediate B88

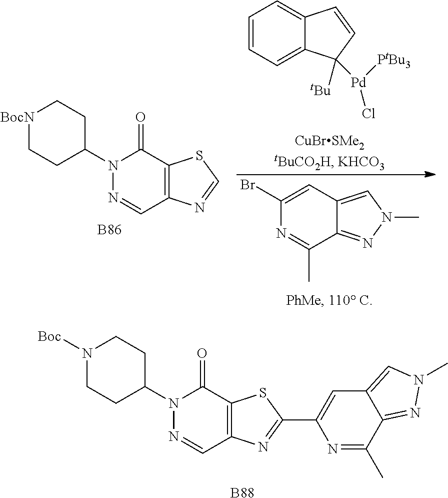

A flame-dried 20 mL sealed tube was charged with tert-butyl 4-(7-oxothiazolo[4,5-d]pyridazin-6(7H)-yl)piperidine-1-carboxylate (123 mg, 0.365 mmol), 5-bromo-2,7-dimethyl-2H-pyrazolo[3,4-c]pyridine (75 mg, 0.33 mmol), pivalic acid (14 mg, 0.13 mmol), potassium bicarbonate (67 mg, 0.66 mmol), and CuBr SMe₂ (14 mg, 66 μmol). The mixture was dissolved in dry toluene (2.2 mL) and argon was bubbled through it for 10 minutes. To the reaction mixture was added chloro(1-tert-butyl-1H-inden-1-yl)(tri-tert-butylphosphine)palladium(II) (8.6 mg, 17 μmol) under flow of argon. The tube was sealed, and the reaction mixture was heated at 110° C. overnight, then diluted with ethyl acetate (25 mL) and filtered through a pad of celite using additional ethyl acetate (10 mL) to wash the filter cake. The filtrate was washed with saturated NaHCO₃ (20 mL) and brine (2×20 mL). The organic phase was dried over Na₂SO₄, filtered, and the filtrate concentrated in vacuo to give a residue. The residue was purified by flash chromatography on a silica gel column using a gradient of 0-100% DCM in ethyl acetate to afford tert-butyl 4-(2-(2,7-dimethyl-2H-pyrazolo[3,4-c]pyridin-5-yl)-7-oxothiazolo[4,5-d]pyridazin-6(7H)-yl)piperidine-1-carboxylate (21 mg, 13%) as a solid. LCMS (ES, m/z): 482.2 [M+H]⁺.

### Synthesis of Compound 255

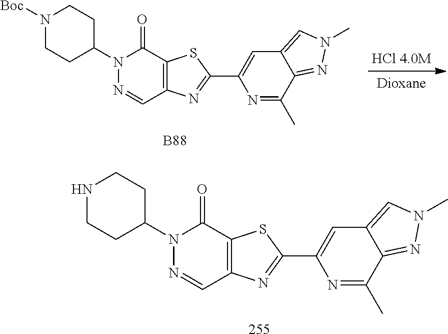 [SMILES: "B88" ; Cc1nc(-c2nc3cnn(C4CCN(C(=O)OC(C)(C)C)CC4)c(=O)c3s2)cc2cn(C)nc12 ; Cc1nc(-c2nc3cnn(C4CCNCC4)c(=O)c3s2)cc2cn(C)nc12 ; "255"]

To tert-butyl 4-(2-(2,7-dimethyl-2H-pyrazolo[3,4-c]pyridin-5-yl)-7-oxothiazolo[4,5-d]pyridazin-6(7H)-yl)piperidine-1-carboxylate 3 (20 mg, 41 μmol) was added HCl 4.0 M in dioxane (1.5 mL). The reaction mixture was stirred vigorously at room temperature for 90 minutes, then concentrated to dryness under reduced pressure to give a residue. The residue was partitioned between DCM (20 mL) and 0.25 M NaOH (20 mL) and stirred to neutralize. The phases were separated, and the aqueous phase was washed with DCM (2×20 mL). The organic phases were combined, dried over Na₂SO₄, filtered, and the filtrate concentrated in vacuo to give a residue.

The residue was purified by flash chromatography on a neutral alumina column using a gradient of 0-10% methanol in DCM to afford 2-(2,7-dimethyl-2H-pyrazolo[3,4-c]pyridin-5-yl)-6-(piperidin-4-yl)thiazolo[4,5-d]pyridazin-7(6H)-one (7.2 mg, 45%) as a solid. LCMS (ES, m/z): 382.1 [M+H]⁺. 1H NMR (CDCl₃, 300 MHz): δ 8.52 (1H, s), 8.46 (1H, s), 8.10 (1H, s), 5.20 (1H, br m), 4.34 (3H, s), 3.28 (2H, d, J=13.1 Hz), 2.97 (3H, s), 2.87 (2H, t, J=12.9 Hz), 1.99 (4H, br m).

### Example 40: Synthesis of Compound 250

### Synthesis of Intermediate B89

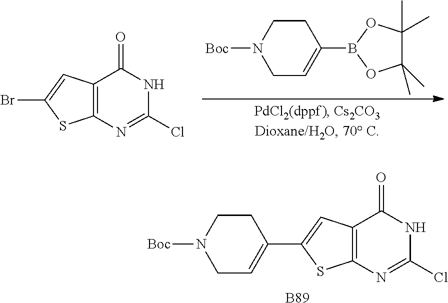

In a sealed tube, a mixture of 6-bromo-2-chlorothieno[2,3-d]pyrimidin-4(3H)-one (400 mg, 1.51 mmol), N-Boc-1,2,3,6-tetrahydropyridine-4-boronicacid pinacol ester (512 mg, 1.66 mmol), PdCl₂(dppf) (110 mg, 0.15 mmol), and Cs₂CO₃ (982 mg, 3.0 mmol) in a mixture of dioxane (10 mL) and water (0.5 mL) was heated at 70° C. overnight under a nitrogen atmosphere. The reaction mixture was filtered over Celite using methanol/DCM (2:8) as eluent. The volatiles were evaporated under reduced pressure to give a residue. The residue was purified by reversed-phase chromatography using a gradient of 30-100% CH₃CN in water to afford tert-butyl 4-(2-chloro-4-oxo-3,4-dihydrothieno[2,3-d]pyrimidin-6-yl)-3,6-dihydropyridine-1(2H)-carboxylate (212 mg, 38%) as a solid. LCMS (ES, m/z): 368.1 [M+H]⁺.

### Synthesis of Intermediate B90

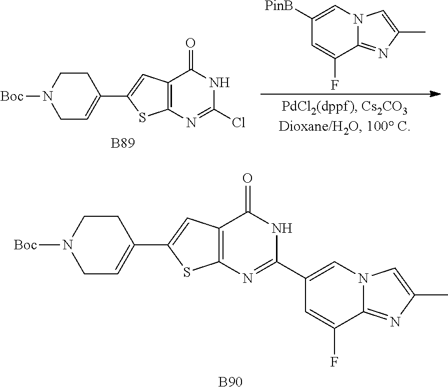

In a sealed tube, a mixture of tert-butyl 4-(2-chloro-4-oxo-3,4-dihydrothieno[2,3-d]pyrimidin-6-yl)-3,6-dihydropyridine-1(2H)-carboxylate (145 mg, 394 μmol), 8-fluoro-2-methyl-6-(4,4,5,5-tetramethyl-1,3,2-dioxaborolan-2-yl)imidazo[1,2-a]pyridine (131 mg, 473 μmol), Cs₂CO₃ (270 mg, 828 μmol), and PdCl₂(dppf) (28.8 mg, 39.4 μmol) in a mixture of 1,4-dioxane (4.0 mL) and water (0.4 mL) was heated at 100° C. overnight under argon atmosphere. The reaction mixture was filtered through Celite using ethyl acetate and DCM as eluent. The volatiles were evaporated under reduced pressure to give a residue. The residue was purified by flash chromatography on a silica gel column using a gradient of methanol from 0 to 10% in DCM to afford tert-butyl 4-(2-(8-fluoro-2-methylimidazo[1,2-a]pyridin-6-yl)-4-oxo-3,4-dihydrothieno[2,3-d]pyrimidin-6-yl)-3,6-dihydropyridine-1(2H)-carboxylate (92.0 mg, 48%) as a solid. LCMS (ES, m/z): 482.1 [M+H]⁺.

### Synthesis of Intermediate B91

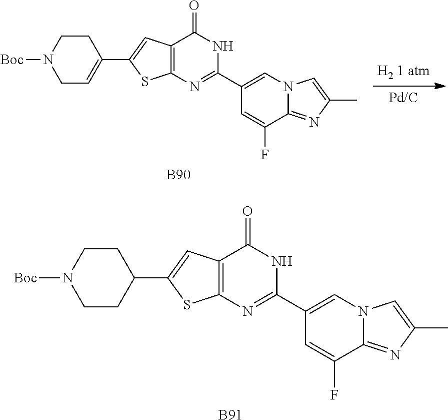 [SMILES: "B90" ; "B91" ; Cc1cn2cc(-c3nc4sc(C5=CCN(C(=O)OC(C)(C)C)CC5)cc4c(=O)[nH]3)cc(F)c2n1 ; Cc1cn2cc(-c3nc4sc(C5CCN(C(=O)OC(C)(C)C)CC5)cc4c(=O)[nH]3)cc(F)c2n1]

To a solution of tert-butyl 4-(2-(8-fluoro-2-methylimidazo[1,2-a]pyridin-6-yl)-4-oxo-3,4-dihydrothieno[2,3-d]pyrimidin-6-yl)-3,6-dihydropyridine-1(2H)-carboxylate (77.0 mg, 160 μmol) in CH₂Cl₂ (9.0 mL) and methanol (3.0 mL) was added palladium on carbon 10 wt. % (66.0 mg). The resulting mixture was stirred at room temperature under H₂ (1 atm) overnight, then filtered through Celite, and the filter cake washed with MeOH/DCM (2:8). The filtrate was concentrated under reduced pressure to give a residue. The residue was purified on a silica gel cartridge using a gradient of methanol from 0 to 10% in CH₂Cl₂ to afford tert-butyl 4-(2-(8-fluoro-2-methylimidazo[1,2-a]pyridin-6-yl)-4-oxo-3,4-dihydrothieno[2,3-d]pyrimidin-6-yl)piperidine-1-carboxylate (47.0 mg, 61%) as a solid. LCMS (ES, m/z): 484.2 [M+H]⁺.

### Synthesis of Compound 250

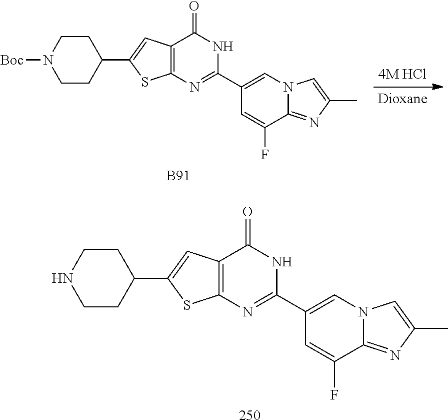 [SMILES: "B91" ; Cc1cn2cc(-c3nc4sc(C5CCN(C(=O)OC(C)(C)C)CC5)cc4c(=O)[nH]3)cc(F)c2n1 ; Cc1cn2cc(-c3nc4sc(C5CCNCC5)cc4c(=O)[nH]3)cc(F)c2n1 ; "250"]

A mixture of tert-butyl 4-(2-(8-fluoro-2-methylimidazo[1,2-a]pyridin-6-yl)-4-oxo-3,4-dihydrothieno[2,3-d]pyrimidin-6-yl)piperidine-1-carboxylate (51.0 mg, 105 μmol) and 4.0 M HCl in dioxane (2.11 mL, 8.44 mmol), in dioxane (5.3 mL) was stirred at room temperature for 2 hours. The reaction mixture was concentrated in vacuo, taken up in CH₂Cl₂ (30 mL), and washed with saturated NaHCO₃ (20 mL). The aqueous phase was dried and the residue extracted with a mixture of methanol and DCM (8:2). The organic layers were combined, dried over Na₂SO₄, filtered, and the filtrate concentrated in vacuo to give a residue. This residue was purified on a silica gel cartridge using a gradient of methanol/NH₄OH (9:1) from 0-20% in CH₂Cl₂ to afford 2-(8-fluoro-2-methylimidazo[1,2-a]pyridin-6-yl)-6-(piperidin-4-yl)thieno[2,3-d]pyrimidin-4(3H)-one (36.5 mg, 90%) as a solid. LCMS (ES, m/z): 384.1 [M+H]⁺. ¹H NMR (CHCl₃-d and CH₃OH-d₄ 0.4+0.1 mL, 400 MHz): δ 8.81 (1H, s), 7.59 (1H, d, J=11.3 Hz), 7.50 (1H, s), 7.15 (1H, s), 3.38 (2H, d, J=12.8 Hz), 3.06 (1H, br, s), 2.94 (2H, t, J=12.7 Hz), 2.39 (3H, s), 2.18 (2H, d, J=13.9 Hz), 1.93 (2H, d, J=13.4 Hz).

### Example 41: Synthesis of Compound 256

### Synthesis of Compound 256

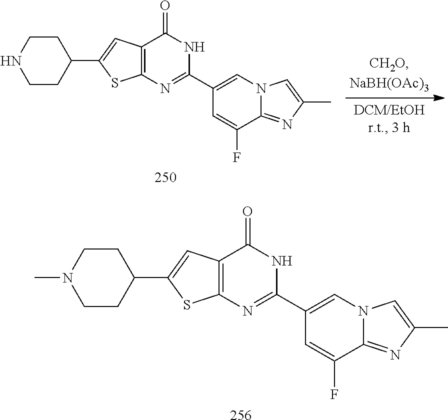

To 2-(8-fluoro-2-methylimidazo[1,2-a]pyridin-6-yl)-6-(piperidin-4-yl)thieno[2,3-d]pyrimidin-4(3H)-one (20.0 mg, 52.2 μmol) dissolved in CH₂Cl₂ (1.0 mL) and ethanol (0.1 mL) was added formaldehyde (37% solution in water, 19.4 μL, 261 μmol). The reaction mixture was stirred at room temperature for 2 hours. To the reaction mixture was added NaBH(OAc)₃ (66.3 mg, 313 μmol), and the reaction mixture was stirred for an additional 2 hours at room temperature. The reaction mixture was concentrated under reduced pressure, then diluted with CH₂Cl₂ (30 mL) and washed with saturated aqueous NaHCO₃ (15 mL). The organic layer was dried over Na₂SO₄ and the solvent was removed in vacuo to give a residue. The residue was purified by flash chromatography on a silica gel column using a gradient of 0 to 20% methanol in CH₂Cl₂ to afford 2-(8-fluoro-2-methylimidazo[1,2-a]pyridin-6-yl)-6-(1-methylpiperidin-4-yl)thieno[2,3-d]pyrimidin-4(3H)-one (18.0 mg, 87%) as a solid. LCMS (ES, m/z): 398.1 [M+H]⁺. ¹H NMR (CHCl₃-d, and CH₃OH-d₄ 0.4+0.1 mL, 400 MHz): δ 8.87 (1H, s), 7.66 (1H, d, J=11.3 Hz), 7.52 (1H, s), 7.18 (1H, s), 3.04 (2H, br s), 2.86 (1H, br s), 2.46 (3H, s), 2.37 (3H, s), 2.22 (2H, br s), 2.09 (2H, d, J=13.1 Hz), 1.96-1.91 (2H, br m).

### Example 42: Synthesis of Compound 251

### Synthesis of Intermediate B92

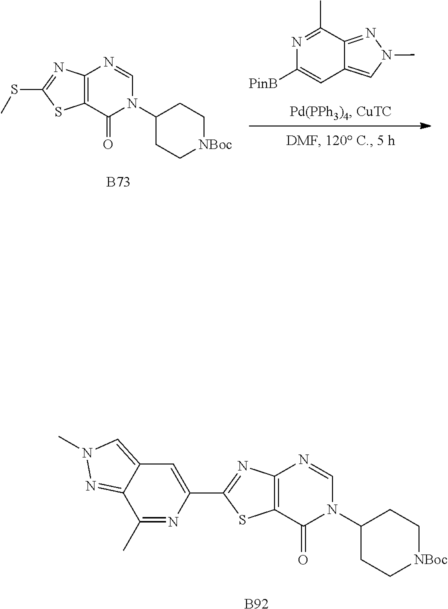

A mixture of tert-butyl 4-(2-(methylthio)-7-oxothiazolo[4,5-d]pyrimidin-6(7H)-yl)piperidine-1-carboxylate (120 mg, 0.31 mmol), 2,7-dimethyl-5-(4,4,5,5-tetramethyl-1,3,2-dioxaborolan-2-yl)-2H-pyrazolo[3,4-c]pyridine (214 mg, 0.78 mmol), CuTC (239 mg, 1.25 mmol) and Pd(PPh₃)₄ (73 mg, 0.063 mmol) in DMF (8.0 mL) was heated to 120° C. for 5 h, then cooled to room temperature. The volatiles were evaporated under reduced pressure. A saturated solution of NaHCO₃ (30 mL) and DCM (30 mL) were added, and the layers were separated. The aqueous layer was extracted with DCM (3×30 mL). The organic layers were combined, dried over Na₂SO₄, filtered, and the filtrate concentrated under reduced pressure to give a residue. The residue was purified by column chromatography on silica gel using a gradient of 0-10% methanol in ethyl acetate to afford tert-butyl 4-(2-(2,7-dimethyl-2H-pyrazolo[3,4-c]pyridin-5-yl)-7-oxothiazolo[4,5-d]pyrimidin-6(7H)-yl)piperidine-1-carboxylate (50 mg, 33%) as a solid.

LCMS (ES, m/z): 482.2 [M+H]⁺.

### Synthesis of Compound 251

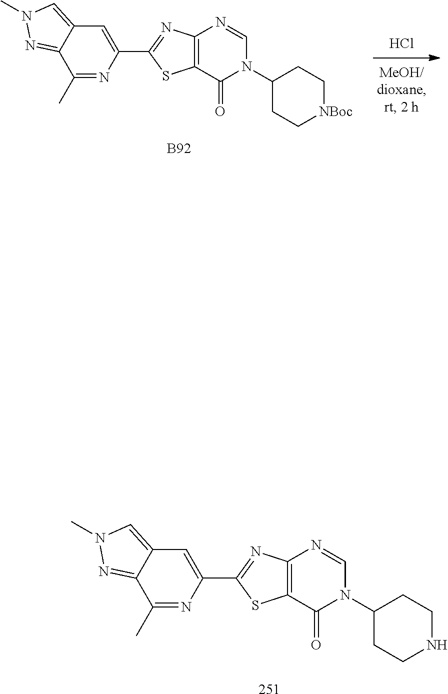 [SMILES: "B92" ; Cc1nc(-c2nc3ncn(C4CCN(C(=O)OC(C)(C)C)CC4)c(=O)c3s2)cc2cn(C)nc12 ; Cl ; Cc1nc(-c2nc3ncn(C4CCNCC4)c(=O)c3s2)cc2cn(C)nc12 ; "251"]

To a solution of tert-butyl 4-(2-(2,7-dimethyl-2H-pyrazolo[3,4-c]pyridin-5-yl)-7-oxothiazolo[4,5-d]pyrimidin-6(7H)-yl)piperidine-1-carboxylate (50 mg, 0.104 mmol) in methanol (6.5 mL) was added a 4 M HCl solution in dioxane (2.2 mL, 8.8 mmol). The reaction mixture was stirred at room temperature for 2 h. The volatiles were evaporated under reduced pressure. An aqueous solution of NaHCO₃ (20 mL) and DCM (30 mL) were added, and the layers were separated. The aqueous layer was extracted with DCM (3×30 mL). The organic layers were combined, dried over Na₂SO₄, filtered, and the filtrate concentrated under reduced pressure to give a residue. The residue was purified by column chromatography on silica gel using a gradient of 0-20% MeOH:Et₃N (2:1 ratio) in DCM. The fractions containing product were collected and concentrated under reduced pressure. Water (10 mL) and DCM (10 mL) were added, and the layers were separated. The aqueous phase was extracted with DCM (3×5 mL). The organic layers were combined, dried over Na₂SO₄, filtered, and the filtrate concentrated under reduced pressure to afford 2-(2,7-dimethyl-2H-pyrazolo[3,4-c]pyridin-5-yl)-6-(piperidin-4-yl)thiazolo[4,5-d]pyrimidin-7(6H)-one (25 mg, 63%) as a solid. LCMS (ES, m/z): 382.1 [M+H]⁺. 1H NMR (CHCl₃-d, 400 MHz): δ_{H} 8.53 (1H, s), 8.28 (1H, s), 8.09 (1H, s), 4.94-5.01 (1H, m), 4.31 (3H, s), 3.24-3.31 (2H, m), 2.94 (3H, s), 2.86 (2H, t, J=12.1 Hz), 1.99-2.08 (2H, m), 1.85-1.97 (2H, m).

### Example 43: Synthesis of Compounds 235, 236, and 257-265

### Synthesis of Intermediate B93

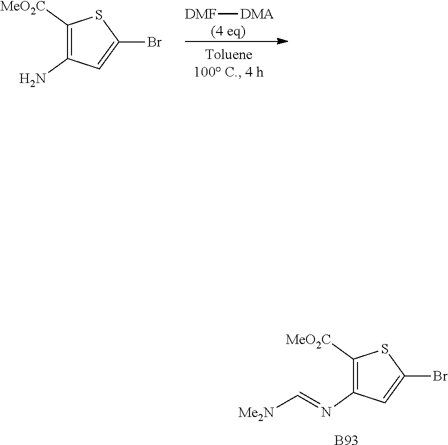

A solution of methyl 3-amino-5-bromothiophene-2-carboxylate (5 g, 20.756 mmol, 1.00 equiv) and DMF-DMA (9.89 g, 83.024 mmol, 4 equiv) in toluene (50 mL) was stirred for 4 h at 100° C. The mixture was allowed to cool to room temperature, then concentrated under vacuum to afford methyl 5-bromo-3-[(E)-[(dimethylamino)methylidene]amino]thiophene-2-carboxylate (5.1 g, 75.95%) as a solid. LCMS (ESI, m/z): 291/293 [M+H]⁺.

### Synthesis of Intermediate B94

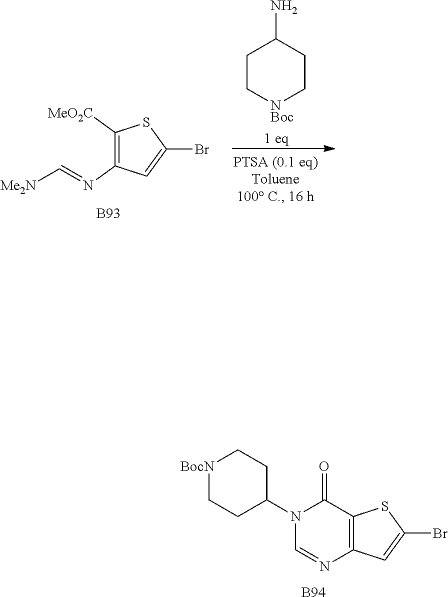 [SMILES: "B93" ; COC(=O)c1sc(Br)cc1/N=C/N(C)C ; "B94" ; CC(C)(C)OC(=O)N1CCC(n2cnc3cc(Br)sc3c2=O)CC1 ; CC(C)(C)OC(=O)N1CCC(N)CC1]

A mixture of methyl 5-bromo-3-[(E)-[(dimethylamino)methylidene]amino]thiophene-2-carboxylate (5.1 g, 17.166 mmol, 1.00 equiv), tert-butyl 4-aminopiperidine-1-carboxylate (3.44 g, 17.166 mmol, 1 equiv) and PTSA (0.30 g, 1.717 mmol, 0.1 equiv) in toluene (50 mL) was stirred for 16 h at 100° C. The reaction mixture was cooled to room temperature. The resulting mixture was concentrated under vacuum to give a residue. The residue was purified by silica gel column chromatography, eluted with PE/EA (5:1) to afford tert-butyl 4-{6-bromo-4-oxothieno[3,2-d]pyrimidin-3-yl}piperidine-1-carboxylate (3 g, 40.07%) as a solid. LCMS (ESI, m z): 414/416 [M+H]⁺.

### Synthesis of Intermediate B95

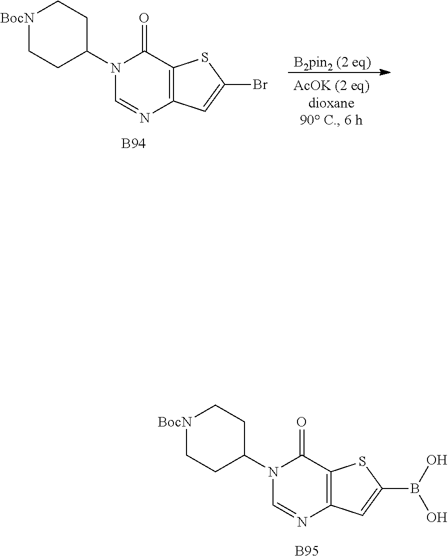 [SMILES: "B94" ; CC(C)(C)OC(=O)N1CCC(n2cnc3cc(Br)sc3c2=O)CC1 ; "B95" ; CC(C)(C)OC(=O)N1CCC(n2cnc3cc(B(O)O)sc3c2=O)CC1]

A mixture of tert-butyl 4-{6-bromo-4-oxothieno[3,2-d]pyrimidin-3-yl}piperidine-1-carboxylate (1.5 g, 3.548 mmol, 1.00 equiv), bis(pinacolato)diboron (1.80 g, 7.096 mmol, 2 equiv), Pd(dppf)Cl₂·CH₂Cl₂ (0.29 g, 0.355 mmol, 0.1 equiv), and AcOK (0.70 g, 7.096 mmol, 2 equiv) in dioxane (45 mL) was stirred for 6 h at 90° C. under nitrogen atmosphere. The reaction mixture was cooled to room temperature, then poured into water (200 mL). The resulting mixture was extracted with ethyl acetate (2×200 mL), dried over anhydrous Na₂SO₄, and filtered. After filtration, the filtrate was concentrated under reduced pressure to give a residue. The residue was re-crystallized from PE/ethyl acetate (5:1 20 mL) to afford 3-[1-(tert-butoxycarbonyl)piperidin-4-yl]-4-oxothieno[3,2-d]pyrimidin-6-ylboronic acid (800 mg, 47.56%) as a solid. LCMS (ESI, m z): 380 [M+H]⁺.

### Synthesis of Intermediate B96

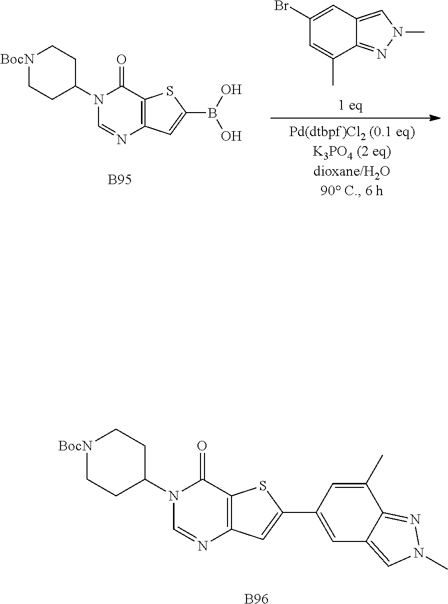 [SMILES: "B95" ; "B96" ; CC(C)(C)OC(=O)N1CCC(n2cnc3cc(B(O)O)sc3c2=O)CC1 ; Cc1cc(-c2cc3ncn(C4CCN(C(=O)OC(C)(C)C)CC4)c(=O)c3s2)cc2cn(C)nc12 ; Cc1cc(Br)cc2cn(C)nc12]

A mixture of 3-[1-(tert-butoxycarbonyl)piperidin-4-yl]-4-oxothieno[3,2-d]pyrimidin-6-ylboronic acid (80 mg, 0.207 mmol, 1.00 equiv), 5-bromo-2,7-dimethylindazole (47 mg, 0.207 mmol, 1 equiv), Pd(dppf)Cl₂·CH₂Cl₂ (17 mg, 0.021 mmol, 0.1 equiv), and K₃PO₄ (88 mg, 0.414 mmol, 2 equiv) in dioxane (3 mL) and water (0.6 mL) was stirred for 6 h at 90° C. under nitrogen atmosphere. The reaction mixture was cooled to room temperature, then poured into water (30 mL). The resulting mixture was extracted with ethyl acetate (2×30 mL), dried over anhydrous Na₂SO₄, and filtered. After filtration, the filtrate was concentrated under reduced pressure to give a residue. The residue was purified by silica gel column chromatography, eluted with PE/EA (1:10) to afford tert-butyl 4-[6-(2,7-dimethylindazol-5-yl)-4-oxothieno[3,2-d]pyrimidin-3-yl]piperidine-1-carboxylate (40 mg, 40.34%) as a solid. LCMS (ESI, m/z): 480 [M+H]⁺.

### Synthesis of Intermediate B97

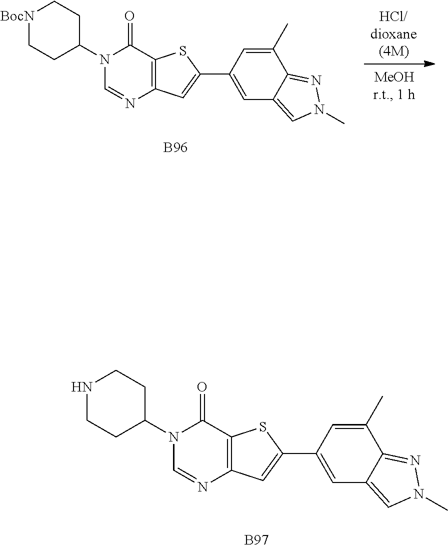 [SMILES: "B96" ; Cc1cc(-c2cc3ncn(C4CCN(C(=O)OC(C)(C)C)CC4)c(=O)c3s2)cc2cn(C)nc12 ; "B97" ; Cc1cc(-c2cc3ncn(C4CCNCC4)c(=O)c3s2)cc2cn(C)nc12]

A mixture of tert-butyl 4-[6-(2,7-dimethylindazol-5-yl)-4-oxothieno[3,2-d]pyrimidin-3-yl]piperidine-1-carboxylate (40 mg, 0.083 mmol, 1.00 equiv) in HCl(gas) in 1,4-dioxane (1 mL) and methanol (0.4 mL) was stirred for 1 h at room temperature. The resulting mixture was concentrated under vacuum, then neutralized to pH 7 with EtN₃, and purified by Chiral-Prep-HPLC (Column, YMC-Actus Triart C18, 30×150 mm, 5 μm; mobile phase, water (10 mmol/L NH₄HCO₃) and acetonitrile (5% ACN up to 60% in 8 min)) to afford 6-(2,7-dimethylindazol-5-yl)-3-(piperidin-4-yl)thieno[3,2-d]pyrimidin-4-one (16.5 mg, 51.98%) as a solid. LCMS (ESI, m z): 380 [M+H]⁺.

### Synthesis of Intermediate B98

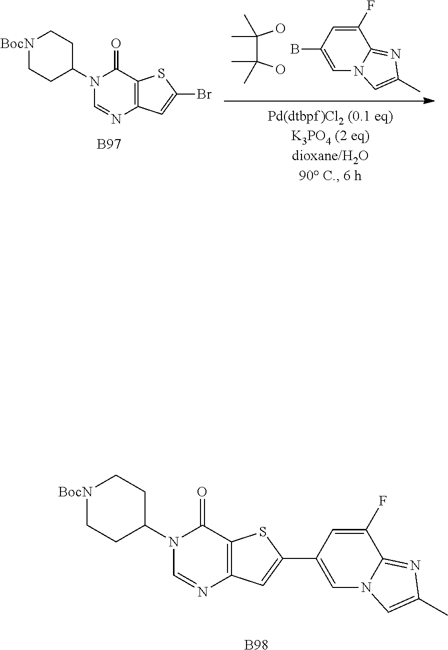

A mixture of tert-butyl 4-{6-bromo-4-oxothieno[3,2-d]pyrimidin-3-yl}piperidine-1-carboxylate (200 mg, 0.473 mmol, 1.00 equiv), 8-fluoro-2-methyl-6-(4,4,5,5-tetramethyl-1,3,2-dioxaborolan-2-yl)imidazo[1,2-a]pyridine (196 mg, 0.710 mmol, 1.5 equiv), Pd(DtBPF)Cl₂ (31 mg, 0.047 mmol, 0.1 equiv), and K₃PO₄ (201 mg, 0.946 mmol, 2 equiv) in dioxane (5 mL) and water (1 mL) was stirred for 6 h at 90° C. under nitrogen atmosphere. The reaction mixture cooled to room temperature, then poured into water (20 mL). The resulting mixture was extracted with ethyl acetate (1×20 mL). A precipitate formed that was collected by filtration and dried under vacuum to afford tert-butyl 4-(6-{8-fluoro-2-methylimidazo[1,2-a]pyridin-6-yl}-4-oxothieno[3,2-d]pyrimidin-3-yl)piperidine-1-carboxylate (110 mg, 43.28%) as a solid. LCMS (ESI, m/z): 484 [M+H]⁺.

### Synthesis of Compound 235

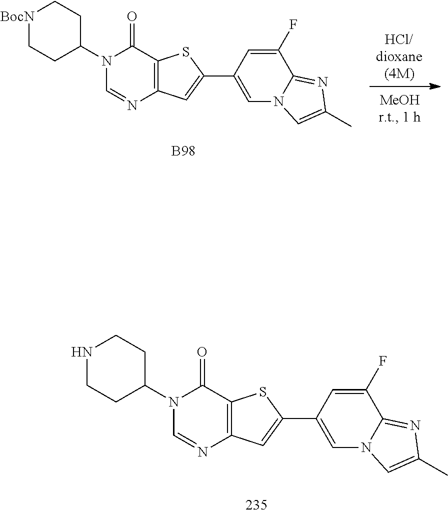 [SMILES: "B98" ; Cc1cn2cc(-c3cc4ncn(C5CCN(C(=O)OC(C)(C)C)CC5)c(=O)c4s3)cc(F)c2n1 ; Cc1cn2cc(-c3cc4ncn(C5CCNCC5)c(=O)c4s3)cc(F)c2n1 ; "235"]

A solution of tert-butyl 4-(6-{8-fluoro-2-methylimidazo[1,2-a]pyridin-6-yl}-4-oxothieno[3,2-d]pyrimidin-3-yl)piperidine-1-carboxylate (110 mg, 0.227 mmol, 1.00 equiv) in HCl (gas) in 1,4-dioxane (2 mL) and methanol (1.1 mL) was stirred for 1 h at room temperature. The resulting mixture was concentrated under vacuum to afford a residue. The residue was purified by Chiral-Prep-HPLC (Column, XBridge Prep OBD C18 Column, 30×150 mm, 5 μm; mobile phase, water (10 mmol/L NH₄HCO₃) and acetonitrile (5% ACN up to 40% in 8 min)) to afford 6-{8-fluoro-2-methylimidazo[1,2-a]pyridin-6-yl}-3-(piperidin-4-yl)thieno[3,2-d]pyrimidin-4-one (22.5 mg, 25.69%) as a solid. LCMS (ESI, m/z): 384 [M+H]⁺.

### Synthesis of Compound 265

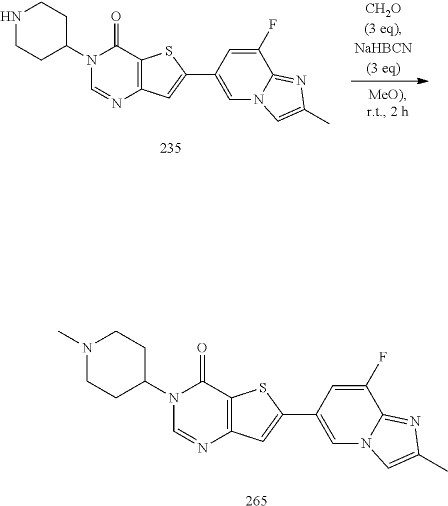

To a stirred mixture of 6-{8-fluoro-2-methylimidazo[1,2-a]pyridin-6-yl}-3-(piperidin-4-yl)thieno[3,2-d]pyrimidin-4-one (50 mg, 0.128 mmol, 1.00 equiv) and formaldehyde (38 mg, 1.280 mmol, 10 equiv) in methanol (1 mL) was added NaBH₃CN (16 mg, 0.256 mmol, 2 equiv) in portions at room temperature under nitrogen. The resulting mixture was stirred for 2 h at room temperature, then concentrated under vacuum to give a residue. The residue was re-crystallized from DMF/methanol (1:1 4 mL), then from MTBE (5 mL) to afford 6-{8-fluoro-2-methylimidazo[1,2-a]pyridin-6-yl}-3-(1-methylpiperidin-4-yl)thieno[3,2-d]pyrimidin-4-one (11.8 mg, 22.37%) as a solid. LCMS (ESI, m/z): 398 [M+H]⁺.

Compounds 235, 236, and 257-265 were prepared according to the procedures outlined herein. outlined in this Example 43 and generalized by Scheme C. The table below provides intermediates used in these procedures and final compound characterization data.

| | | LCMS | |
|---|---|---|---|
| | Coupling | (ESI, m/z) | |
| Compound No. and Structure | Reagent | [M + H]⁺ | ¹H NMR δ |
| 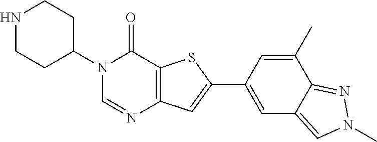 [SMILES: Cc1cc(-c2cc3ncn(C4CCNCC4)c(=O)c3s2)cc2cn(C)nc12] 236 | 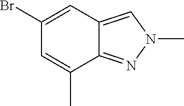 | 380 | (400 MHz, DMSO-d₆) δ 8.46 (d, J = 16.4 Hz, 2H), 8.06 (d, J = 1.9 Hz, 1H), 7.77 (s, 1H), 7.53 (d, J = 1.7 Hz, 1H), 4.71 (tt, J = 12.0, 3.9 Hz, 1H), 4.20 (s, 3H), 3.10 (d, J = 12.7 Hz, 2H), 2.62 (td, J = 12.3, 2.5 Hz, 2H), 2.59 - 2.54 (s, 3H), 1.93 (qd, J = 11.7, 3.8 Hz, 2H), 1.80 (d, J = 9.9 Hz, |
| | | | 2H) |
| 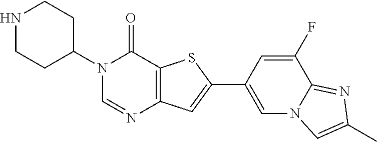 [SMILES: Cc1cn2cc(-c3cc4ncn(C5CCNCC5)c(=O)c4s3)cc(F)c2n1] 235 | 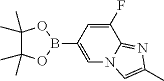 | 384 | (400 MHz, DMSO-d₆) δ 8.92 (d, J = 1.6 Hz, 1H), 8.42 (s, 1H), 7.84 (d, J = 3.1 Hz, 1H), 7.79 (s, 1H), 7.57 (dd, J = 12.1, 1.6 Hz, 1H), 4.70 (tt, J = 12.1, 4.0 Hz, 1H), 3.13 (dt, J = 12.7, 3.0 Hz, 2H), 2.71 - 2.60 (m, 2H), 2.39 (s, 3H), 1.95 (qd, J = 12.0, 4.1 Hz, 2H), 1.87 − 1.78 (m, 2H) |
| 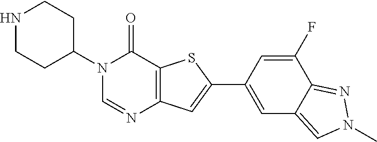 [SMILES: Cn1cc2cc(-c3cc4ncn(C5CCNCC5)c(=O)c4s3)cc(F)c2n1] 257 | 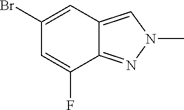 | 384 | (400 MHz, DMSO-d₆) δ 8.61 (s, 1H), 8.51 (s, 1H), 8.00 − 7.94 (m, 2H), 7.40 (dd, J = 11.6, 1.3 Hz, 1H), 4.71 (tt, J = 12.1, 4.0 Hz, 1H), 4.22 (s, 3H), 3.13 − 3.05 (m, 2H), 2.61 (td, J = 12.2, 2.5 Hz, 2H), 2.22 (s, 1H), 1.92 (qd, J = 11.9, 4.0 Hz, 2H), 1.83 − 1.75 (m, 2H) |
| 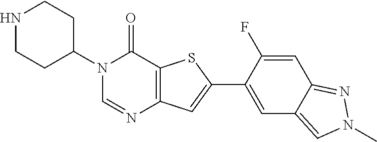 [SMILES: Cn1cc2cc(-c3cc4ncn(C5CCNCC5)c(=O)c4s3)c(F)cc2n1] 258 | 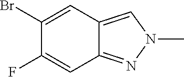 | 384 | (400 MHz, DMSO-d₆) δ 8.53 (d, J = 11.4 Hz, 2H), 8.38 (d, J = 7.7 Hz, 1H), 7.75 (d, J = 1.5 Hz, 1H), 7.58 (d, J = 12.8 Hz, 1H), 4.71 (tt, J = 12.2, 3.9 Hz, 1H), 4.20 (s, 3H), 3.09 (d, J = 12.1 Hz, 2H), 2.67 − 2.56 (m, 2H), 2.13 (s, 1H), 1.92 (qd, J = 11.9, 4.1 Hz, 2H), 1.79 (d, J = 10.6 Hz, 2H) |
| 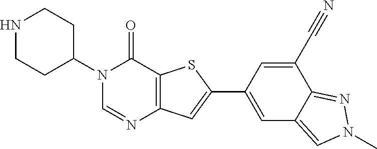 [SMILES: Cn1cc2cc(-c3cc4ncn(C5CCNCC5)c(=O)c4s3)cc(C#N)c2n1] 259 | 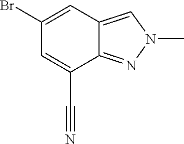 | 391 | (400 MHz, DMSO-d₆) δ 8.93 (d, J = 11.2 Hz, 1H), 8.74 (s, 1H), 8.72 − 8.65(br, 1H), 8.63 (d, J = 1.7 Hz, 1H), 8.50 (d, J = 1.7 Hz, 1H), 8.36 (s, 1H), 8.00 (s, 1H), 4.88 (ddt, J = 12.1, 7.5, 3.9 Hz, 1H), 4.29 (s, 3H), 3.46 (d, J = 12.5 Hz, 2H), 3.12 (q, J = 11.7 Hz, 2H), 2.39 (td, J = 12.9, 4.0 Hz, |
| | | | 2H), 2.14 − 2.05 (m, 2H) |
| 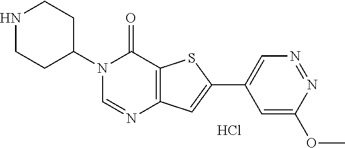 [SMILES: COc1cc(-c2cc3ncn(C4CCNCC4)c(=O)c3s2)cnn1 ; Cl] 260 | 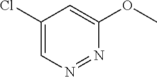 | 344 | (400 MHz, DMSO-d₆) δ 9.46 (d, J = 1.9 Hz, 1H), 8.73 (s, 2H), 8.41 (s, 1H), 8.35 (s, 1H), 7.69 (d, J = 1.9 Hz, 1H), 4.94 − 4.83 (m, 1H), 4.11 (s, 3H), 3.46 (d, J = 12.5 Hz, 2H), 3.11 (td, J = 14.3, 13.5, 3.3 Hz, 2H), 2.44 − 2.30 (m, 2H), 2.14 − 2.06 (m, 2H) |
| 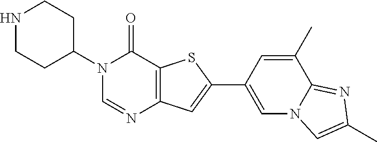 [SMILES: Cc1cn2cc(-c3cc4ncn(C5CCNCC5)c(=O)c4s3)cc(C)c2n1] 261 | 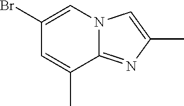 | 380 | (400 MHz, DMSO-d₆) δ 8.98 (s, 1H), 8.51 (s, 1H), 7.81 (d, J = 1.2 Hz, 1H), 7.72 (s, 1H), 7.51 (d, J = 1.8 Hz, 1H), 4.75-4.65 (m, 1H), 3.09 (d, J = 12.7 Hz, 2H), 2.66-2.55 (m, 2H), 2.57 (s, 3H), 2.36 (s, 3H), 1.91 (qd, J = 12.0, 4.0 Hz, 2H), 1.79 (d, J = 11.6 Hz, 2H) |
| 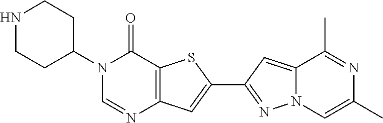 [SMILES: Cc1cn2nc(-c3cc4ncn(C5CCNCC5)c(=O)c4s3)cc2c(C)n1] 262 | 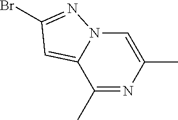 | 381 | (400 MHz, DMSO-d₆) δ 8.54 (d, J = 14.9 Hz, 2H), 7.97 (s, 1H), 7.63 (d, J = 1.0 Hz, 1H), 4.71 (tt, J = 12.2, 4.0 Hz, 1H), 3.09 (d, J = 12.2 Hz, 2H), 2.71 (s, 3H), 2.61 (td, J = 12.2, 2.5 Hz, 2H), 2.43 (s, 3H), 1.92 (qd, J = 11.8, 3.9 Hz, 2H), 1.79 (d, J = 11.3 Hz, 2H) |
| 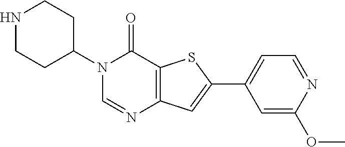 [SMILES: COc1cc(-c2cc3ncn(C4CCNCC4)c(=O)c3s2)ccn1] 263 | 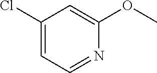 | 343 | (400 MHz, DMSO-d₆) δ 8.54 (s, 1H), 8.29 (d, J = 5.4 Hz, 1H), 8.14 (s, 1H), 7.46 (dd, J = 5.4, 1.6 Hz, 1H), 7.30 (d, J = 1.7 Hz, 1H), 4.70 (tt, J = 12.1, 3.9 Hz, 1H), 3.92 (s, 3H), 3.13 − 3.05 (m, 2H), 2.61 (td, J = 12.2, 2.5 Hz, 2H), 1.92 (qd, J = 12.0, 4.0 Hz, 2H), 1.83- 1.75 (m, 2H) |
| 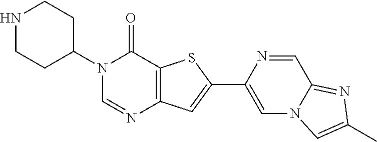 [SMILES: Cc1cn2cc(-c3cc4ncn(C5CCNCC5)c(=O)c4s3)ncc2n1] 264 | 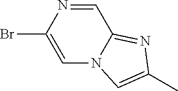 | 367 | (400 MHz, DMSO-d₆) δ 9.25 (d, J = 1.5 Hz, 1H), 8.93 (d, J = 1.8 Hz, 1H), 8.38 (s, 1H), 7.91 (s, 1H), 7.83 (s, 1H), 4.69 (tt, J = 12.0, 4.1 Hz, 1H), 3.13 − 3.04 (m, 2H), 2.66 − 2.55 (m, 2H), 2.46 (s, 3H), 1.90 (qd, J = 11.9, 4.1 Hz, 2H), 1.84 − 1.77 (m, 2H) |
| 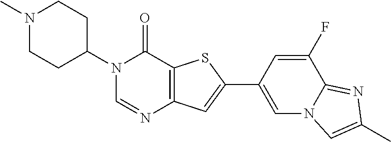 | 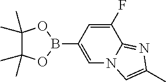 | 398 | (400 MHz, DMSO-d₆) δ 8.94 (d, J = 1.6 Hz, 1H), 8.44 (s, 1H), 7.84 (d, J = 3.1 Hz, 1H), 7.80 (s, 1H), 7.57 (dd, J = 12.0, 1.6 Hz, 1H), 4.62 (s, 1H), 2.99 (m, 2H), 2.39 (s, 3H), 2.31 (s, 3H), 2.20 (s, 4H), 1.88 (t, J = 11.3 Hz, 2H) |
| 265 | | | |

### Example 44: Synthesis of Compounds 335 and 336

### Synthesis of Intermediate C1

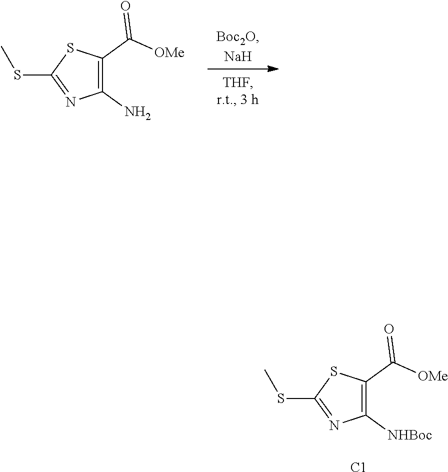 [SMILES: COC(=O)c1sc(SC)nc1N ; COC(=O)c1sc(SC)nc1NC(=O)OC(C)(C)C ; "C1"]

A solution of methyl 4-amino-2-(methylthio)thiazole-5-carboxylate (3.6 g, 1 equiv) in THF (75 mL) was treated with NaH (2.77 g, 69.256 mmol, 4.72 equiv, 60%) for 30 min at room temperature, followed by the addition of Boc₂O (4.8 g, 1.2 equiv) in THF (60 mL) at rt. The mixture was stirred for 3 h at room temperature. Desired product could be detected by LCMS.

The reaction was quenched with saturated aqueous NH₄Cl at room temperature. The resulting mixture was extracted with EA (3×100 mL). The combined organic layers were washed with brinr (3×50 mL), dried over anhydrous Na₂SO₄. After filtration, the filtrate was concentrated under reduced pressure. The residue was purified by silica gel column chromatography, eluted with PE:EA (20:1-5:1) to afford methyl 4-((tert-butoxycarbonyl)amino)-2-(methylthio)thiazole-5-carboxylate (3.5 g) as a solid.

### Synthesis of Intermediate C2

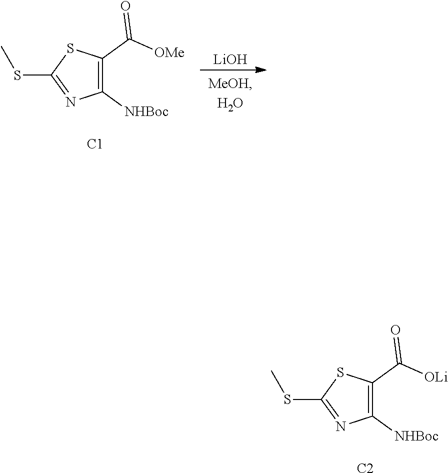

A solution of methyl 4-((tert-butoxycarbonyl)amino)-2-(methylthio)thiazole-5-carboxylate (1 g, 3.289 mmol, 1.00 equiv), LiOH (0.16 g, 6.578 mmol, 2 equiv) in methanol (10 mL) and H₂O (10 mL) was stirred at 60° C. for 3 h. The reaction was monitored by TLC. lithium 4-((tert-butoxycarbonyl)amino)-2-(methylthio)thiazole-5-carboxylate (0.973 g, 99.93%), The crude product was used in the next step directly without further purification. LCMS (ES, m/z):290 [M+H]⁺

### Synthesis of Intermediate C3

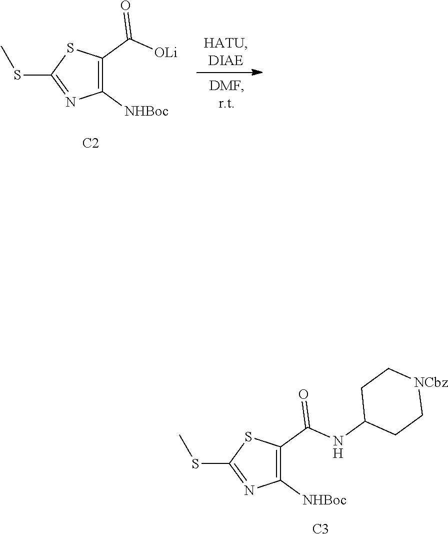

A mixture of lithium 4-((tert-butoxycarbonyl)amino)-2-(methylthio)thiazole-5-carboxylate (1.16 g, 3.370 mmol, 1.00 equiv, 86%), HATU (1.54 g, 4.040 mmol, 1.2 equiv) and benzyl 4-aminopiperidine-1-carboxylate (0.95 g, 4.040 mmol, 1.2 equiv) in DMF (15 mL) was treated with DIEA (0.65 g, 5.050 mmol, 1.5 equiv) at room temperature. The mixture was stirred for 5 h at room temperature. The reaction was quenched with H₂O (50 mL) at rt. The resulting mixture was extracted with EA (3×30 mL). The combined organic layers were washed with H₂O (5×20 mL) and brine (4×20 mL), dried over anhydrous sodium sulfate. After filtration, the filtrate was concentrated under reduced pressure. The residue was purified by silica gel column chromatography, eluted with PE:EA (5:1˜1:1) to afford benzyl 4-(4-((tert-butoxycarbonyl)amino)-2-(methylthio)thiazole-5-carboxamido)piperidine-1-carboxylate (0.83 g, 49%) as a solid. LCMS (ES, m/z): 507 [M+H]⁺

### Synthesis of Intermediate C4

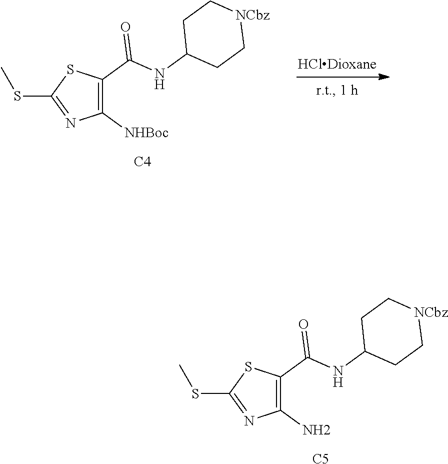

A solution of benzyl 4-{4-[(tert-butoxycarbonyl)amino]-2-(methylsulfanyl)-1,3-thiazole-5-amido}piperidine-1-carboxylate (830 mg, 1.638 mmol, 1 equiv) and HCl(gas) in 1,4-dioxane (3 mL) in dioxane (3 mL) was stirred for 3 h at room temperature. The resulting mixture was concentrated under vacuum. The mixture was basified to pH 9 with saturated aqueous NaHCO₃ at 0° C. The resulting mixture was extracted with DCM (3×50 mL). The combined organic layers were dried over anhydrous Na₂SO₄. After filtration, the filtrate was concentrated under reduced pressure. This resulted in benzyl 4-[4-amino-2-(methylsulfanyl)-1,3-thiazole-5-amido]piperidine-1-carboxylate (650 mg, 98%) as a solid. LCMS (ES, m/z): 407 [M+H]+

### Synthesis of Intermediate C6

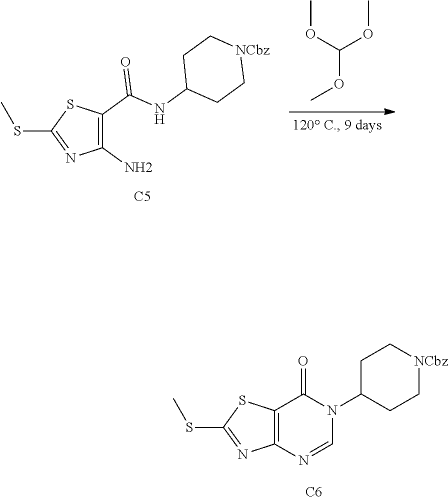

A solution of benzyl 4-[4-amino-2-(methylsulfanyl)-1,3-thiazole-5-amido]piperidine-1-carboxylate (600 mg, 1.476 mmol, 1 equiv) in trimethyl orthoformate (3 mL, 0.028 mmol, 0.02 equiv) was stirred for 9 overnight at 120° C. The mixture was allowed to cool down to room temperature. The reaction was quenched by the addition of water (30 mL). The resulting mixture was extracted with EA (3×50 mL). The combined organic layers were washed with saturated aqueous NaCl (100 mL), dried over anhydrous sodium sulfate. After filtration, the filtrate was concentrated under reduced pressure. The residue was purified by silica gel column chromatography, eluted with PE/EA (2:3) to afford benzyl 4-[2-(methylsulfanyl)-7-oxo-[1,3]thiazolo[4,5-d]pyrimidin-6-yl]piperidine-1-carboxylate (380 mg, 62%) as a solid.

LCMS (ES, m/z): 417 [M+H]⁺

### Synthesis of Intermediate C7

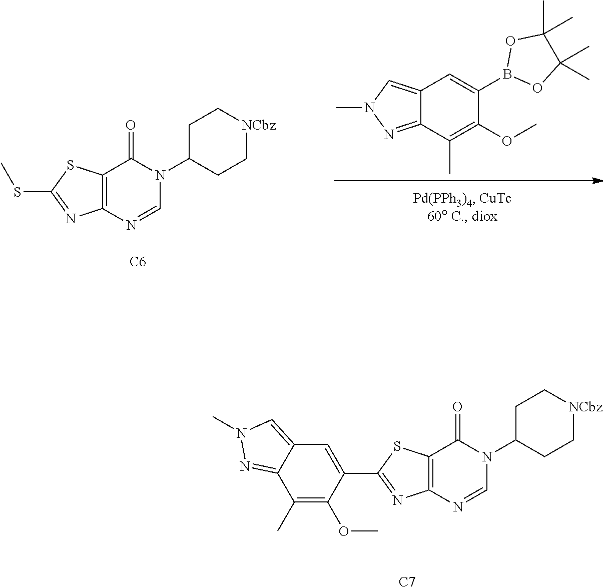

Into an 8-mL vial, was added benzyl 4-[2-(methylsulfanyl)-7-oxo-[1,3]thiazolo[4,5-d]pyrimidin-6-yl]piperidine-1-carboxylate (100 mg, 0.240 mmol, 1 equiv), 6-methoxy-2,7-dimethyl-5-(4,4,5,5-tetramethyl-1,3,2-dioxaborolan-2-yl)indazole (108.83 mg, 0.360 mmol, 1.5 equiv), Pd(PPh₃)₄ (55.49 mg, 0.048 mmol, 0.2 equiv), CuTc (9.16 mg, 0.048 mmol, 0.2 equiv) and dioxane (4 mL). The reaction mixture was evacuated and flushed three times with nitrogen.

The resulting mixture was stirred for 2 overnight at 60° C. The mixture was allowed to cool down to room temperature. The reaction was quenched by the addition of water (20 mL). The resulting mixture was extracted with EA (3×20 mL). The combined organic layers were washed with saturated aqueous NaCl (1×30 mL), dried over anhydrous sodium sulfate. After filtration, the filtrate was concentrated under reduced pressure. The residue was purified by silica gel column chromatography, eluted with DCM/MeOH (5:1) to afford benzyl 4-[2-(6-methoxy-2,7-dimethylindazol-5-yl)-7-oxo-[1,3]thiazolo[4,5-d]pyrimidin-6-yl]piperidine-1-carboxylate (40 mg, 31%) as an oil. LCMS (ES, m/z): 545 [M+H]⁺

### Synthesis of Compound 335

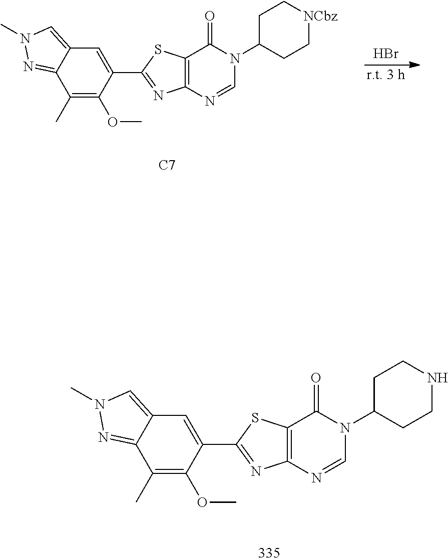 [SMILES: COc1c(-c2nc3ncn(C4CCN(C(=O)OCc5ccccc5)CC4)c(=O)c3s2)cc2cn(C)nc2c1C ; Br ; "C7" ; COc1c(-c2nc3ncn(C4CCNCC4)c(=O)c3s2)cc2cn(C)nc2c1C ; "335"]

Into a 8 mL vial, were added benzyl 4-[2-(6-methoxy-2,7-dimethylindazol-5-yl)-7-oxo-[1,3]thiazolo[4,5-d]pyrimidin-6-yl]piperidine-1-carboxylate (40 mg, 0.073 mmol, 1 equiv) and HBr in water (3 mL). The resulting mixture was stirred for 3 h at room temperature. The mixture was basified to pH 9 with saturated aqueous NaHCO₃ at 0° C. The resulting mixture was concentrated under vacuum. The residue was washed with MeOH. The crude product was purified by Prep-HPLC with the following conditions: Column: XBridge Shield RP18 OBD Column, 19*150 mm, 5 m; Mobile Phase A: Water (10 mmol/L NH₄HCO₃), Mobile Phase B: ACN; Flow rate: 60 mL/min; Gradient: 5% B to 40% B in 8 min, 40% B; Wave Length: UV 220 nm; RT1(min): 6.50. product was obtained. This resulted in 2-(6-methoxy-2,7-dimethylindazol-5-yl)-6-(piperidin-4-yl)-[1,3]thiazolo[4,5-d]pyrimidin-7-one (15 mg, 49%) as a solid. LCMS (ES, m/z): 411 [M+H]⁺¹H-NMR (400 MHz, DMSO-d₆) δ 8.71 (d, J=0.8 Hz, 1H), 8.61 (s, 1H), 8.57 (s, 1H), 4.70 (td, J=10.3, 8.3, 5.8 Hz, 1H), 4.20 (s, 3H), 3.86 (s, 3H), 3.11 (d, J=12.4 Hz, 2H), 2.63 (t, J=11.5 Hz, 2H), 2.53 (s, 3H), 2.02-1.88 (m, 2H), 1.81 (d, J=11.2 Hz, 2H).

### Synthesis of Compound 336

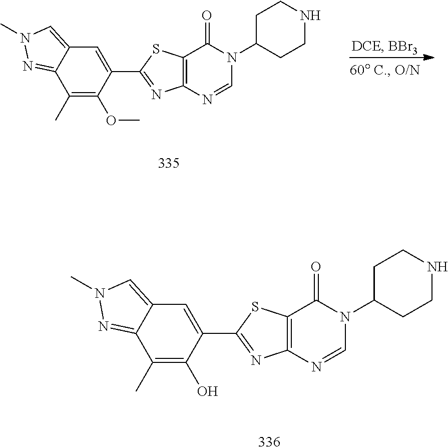 [SMILES: COc1c(-c2nc3ncn(C4CCNCC4)c(=O)c3s2)cc2cn(C)nc2c1C ; "335" ; Cc1c(O)c(-c2nc3ncn(C4CCNCC4)c(=O)c3s2)cc2cn(C)nc12 ; "336"]

Into a 8 mL vial, were added 2-(6-methoxy-2,7-dimethylindazol-5-yl)-6-(piperidin-4-yl)-[1,3]thiazolo[4,5-d]pyrimidin-7-one (10 mg, 0.024 mmol, 1 equiv), DCE (0.5 mL) and BBr₃ (0.5 mL, 5.289 mmol, 217.11 equiv). The resulting solution was stirred for overnight at 60° C. The mixture was basified to pH 9 with NH₃-MeOH at 0° C. The resulting mixture was concentrated under vacuum. The crude product was purified by Prep-HPLC with the following conditions: Column: YMC-Actus Triart C18, 30*150 mm, 5 m; Mobile Phase A: Water (10 mmol/L NH₄HCO₃), Mobile Phase B: ACN; Flow rate: 60 mL/min; Gradient: 5% B to 35% B in 10 min, 35% B; Wave Length: UV 220 nm; RT1(min): 9.17; product was obtained. This resulted in 2-(6-hydroxy-2,7-dimethylindazol-5-yl)-6-(piperidin-4-yl)-[1,3]thiazolo[4,5-d]pyrimidin-7-one (5.8 mg, 59%) as a solid. LCMS (ES, m/z): 397 [M+H]^{+ 1}H-NMR (400 MHz, DMSO-d₆) δ 8.53 (s, 2H), 8.38 (s, 1H), 5.05-4.43 (m, 1H), 4.08 (s, 3H), 3.03 (d, J=12.1 Hz, 2H), 2.55 (t, J=11.9 Hz, 2H), 2.34 (s, 3H), 1.86 (dd, J=12.3, 3.9 Hz, 2H), 1.74 (d, J=11.7 Hz, 2H).

### Example 45: Synthesis of Compound 802

### Synthesis of Intermediate C8

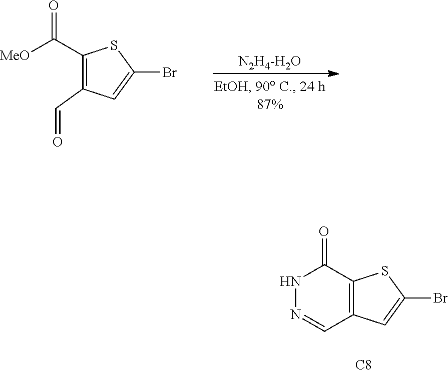 [SMILES: COC(=O)c1sc(Br)cc1C=O ; "N2H4-H2O" ; O=c1[nH]ncc2cc(Br)sc12 ; "C8"]

To a solution of methyl 5-bromo-3-formylthiophene-2-carboxylate (62 mg, 0.25 mmol) in EtOH (2.0 mL) was added a solution of hydrazine hydrate (50% in H₂O, 40 μL, 0.40 mmol). The reaction mixture was heated to 90° C. for 24 h and then cooled to rt. The volatiles were evaporated under reduced pressure. The crude product was purified by column chromatography on silica gel using a gradient of 0-10% MeOH in DCM to afford C₈ (50 mg, 87%) as a solid. LCMS (ES, m/z): 231.0 [M+H]⁺.

### Synthesis of Intermediate C9

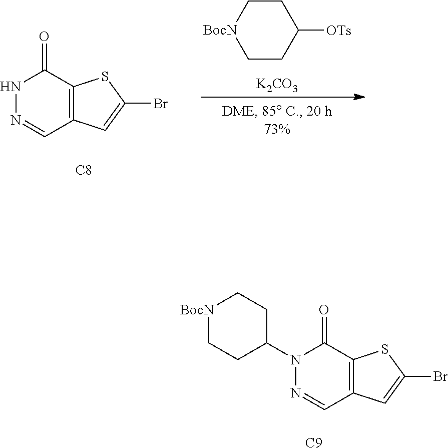

A mixture of 2-bromothieno[2,3-d]pyridazin-7(6H)-one (50 mg, 0.217 mmol), tert-butyl 4-(tosyloxy)piperidine-1-carboxylate (231 mg, 0.65 mmol) and K₂CO₃ (60 mg, 0.43 mmol) in DME (1.7 mL) was heated to 85° C. for 20 h and then cooled to rt. The reaction mixture was filtered, and the volatiles were evaporated under reduced pressure. Water (10 mL) and DCM (10 mL) were added, and the layers were separated. The aqueous layer was extracted with DCM (3×10 mL). The organic layers were combined, dried over Na₂SO₄, filtered and concentrated under reduced pressure. The crude product was purified by column chromatography on silica gel using a gradient of 0-100% EtOAc in hexane to afford tert-butyl 4-(2-bromo-7-oxothieno[2,3-d]pyridazin-6(7H)-yl)piperidine-1-carboxylate (65 mg, 73%) as a solid. LCMS (ES, m/z): 414.0 [M+H]⁺.

### Synthesis of Intermediate C10

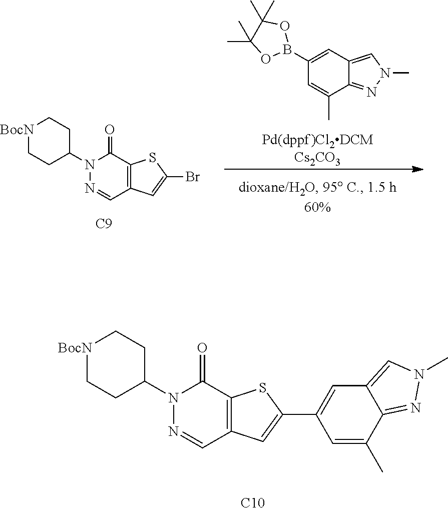

A mixture of tert-butyl 4-(2-bromo-7-oxothieno[2,3-d]pyridazin-6(7H)-yl)piperidine-1-carboxylate (65 mg, 0.157 mmol), 2,7-dimethyl-5-(4,4,5,5-tetramethyl-1,3,2-dioxaborolan-2-yl)-2H-indazol (65 mg, 0.24 mmol), Cs₂CO₃ (102 mg, 0.31 mmol) and Pd(dppf)Cl₂·DCM (13 mg, 0.016 mmol) in a mixture of dioxane (1.0 mL) and H₂O (0.1 mL) was heated to 90° C. for 1.5 h and then cooled to rt. The reaction mixture was filtered over Celite using 10% MeOH in DCM as eluent. The volatiles were evaporated under reduced pressure. Water (10 mL) and DCM (10 mL) were added, and the layers were separated. The aqueous layer was extracted with DCM (3×10 mL). The organic layers were combined, dried over Na₂SO₄, filtered and concentrated under reduced pressure. The crude product was purified by column chromatography on silica gel using a gradient of 0-10% MeOH in EtOAc to afford tert-butyl 4-(2-(2,7-dimethyl-2H-indazol-5-yl)-7-oxothieno[2,3-d]pyridazin-6(7H)-yl)piperidine-1-carboxylate (45 mg, 60%) as a solid. LCMS (ES, m/z): 480.2 [M+H]⁺.

### Synthesis of Compound 802

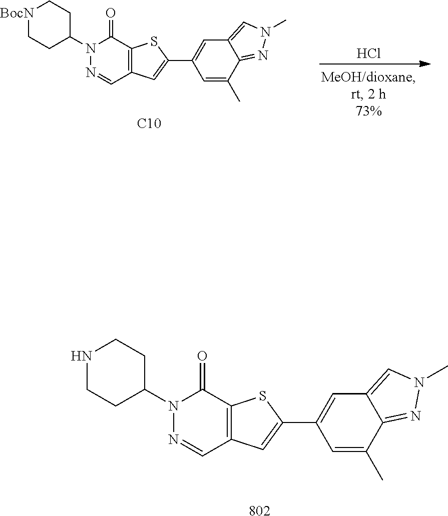 [SMILES: Cc1cc(-c2cc3cnn(C4CCN(C(=O)OC(C)(C)C)CC4)c(=O)c3s2)cc2cn(C)nc12 ; Cl ; "C10" ; Cc1cc(-c2cc3cnn(C4CCNCC4)c(=O)c3s2)cc2cn(C)nc12 ; "802"]

To a solution of tert-butyl 4-(2-(2,7-dimethyl-2H-indazol-5-yl)-7-oxothieno[2,3-d]pyridazin-6(7H)-yl)piperidine-1-carboxylate (40 mg, 0.083 mmol) in MeOH (5.0 mL) was added 4 M HCl solution in dioxane (1.8 mL, 7.1 mmol). The reaction mixture was stirred at rt for 2 h. The volatiles were evaporated under reduced pressure. A saturated solution of NaHCO₃ (20 mL), KOH (0.1 M, 10 mL) and DCM (30 mL) were added, and the layers were separated. The aqueous layer was extracted with DCM (3×30 mL). The organic layers were combined, dried over Na₂SO₄, filtered and concentrated under reduced pressure. The crude product was purified by column chromatography on silica gel using a gradient of 0-15% MeOH:Et₃N (2:1 ratio) in DCM. The fractions containing product were collected and concentrated under reduced pressure. Water (5 mL) and DCM (5 mL) were added, and the layers were separated. The aqueous phase was extracted with DCM (2×5 mL). The organic layers were combined, dried over Na₂SO₄, filtered and concentrated under reduced pressure to afford of 2-(2,7-dimethyl-2H-indazol-5-yl)-6-(piperidin-4-yl)thieno[2,3-d]pyridazin-7(6H)-one (23 mg, 73%) as a solid. LCMS (ES, m/z): 380.2 [M+H]⁺. 1H NMR (DMSO-d₆, 400 MHz): δ_{H} 8.46 (1H, s), 8.45 (1H, s), 8.05 (1H, s), 7.86 (1H, s), 7.50 (1H, s), 4.94 (1H, t, J=11.4 Hz), 4.20 (3H, s), 3.06 (2H, d, J=12.3 Hz), 2.56-2.63 (5H, m), 1.81-1.89 (2H, m), 1.71 (2H, d, J=11.7 Hz).

### Example 46: Synthesis of Compounds 804 and 808

### Synthesis of Intermediate C11

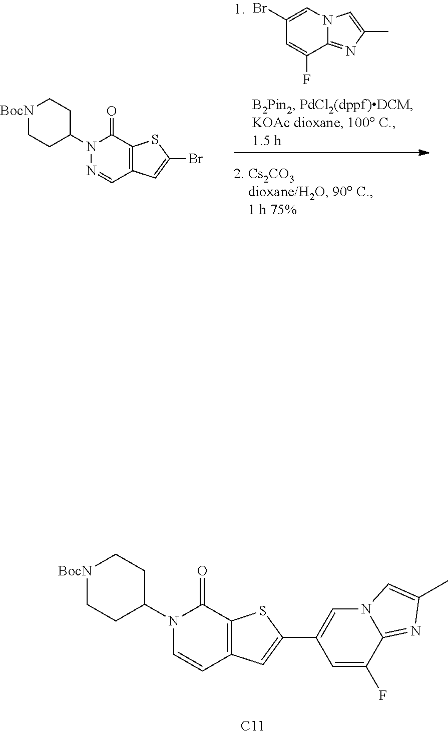 [SMILES: Cc1cn2cc(Br)cc(F)c2n1 ; CC(C)(C)OC(=O)N1CCC(n2ncc3cc(Br)sc3c2=O)CC1 ; Cc1cn2cc(-c3cc4ccn(C5CCN(C(=O)OC(C)(C)C)CC5)c(=O)c4s3)cc(F)c2n1 ; "C11"]

A mixture of 6-bromo-8-fluoro-2-methylimidazo[1,2-a]pyridine (180 mg, 0.786 mmol), Bis(pinacolato)diboron (202 mg, 0.786 mmol), PdCl₂(dppf).DCM (44 mg, 0.060 mmol), and KOAc (180 mg, 1.84 mmol) in dioxane (4.0 mL) was heated to 100° C. for 1.5 h. A solution of tert-butyl 4-(2-bromo-7-oxothieno[2,3-c]pyridin-6(7H)-yl)piperidine-1-carboxylate 2 (See, 250 mg, 0.606 mmol) in dioxane (4.0 mL), Cs₂CO₃ (590 mg, 1.81 mmol) and H₂O (0.8 mL) were sequentially added under argon. The reaction mixture was heated at 90° C. for 1 h and then cooled to rt. The reaction mixture was filtered over Celite using 20% MeOH in DCM as eluent. The volatiles were evaporated under reduced pressure. Water (20 mL) and DCM (20 mL) were added, and the layers were separated. The aqueous layer was extracted with DCM (3×20 mL). The organic layers were combined, dried over Na₂SO₄, filtered and concentrated under reduced pressure. The crude product was purified by column chromatography on silica gel using a gradient of 0-100% EtOAc in hexane to afford tert-butyl 4-(2-(8-fluoro-2-methylimidazo[1,2-a]pyridin-6-yl)-7-oxothieno[2,3-c]pyridin-6(7H)-yl)piperidine-1-carboxylate (220 mg, 75%) as a solid.

LCMS (ES, m/z): 483.2 [M+H]⁺.

### Synthesis of Compound 804

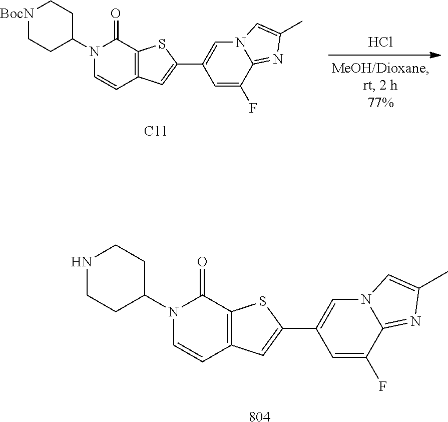 [SMILES: Cc1cn2cc(-c3cc4ccn(C5CCN(C(=O)OC(C)(C)C)CC5)c(=O)c4s3)cc(F)c2n1 ; Cl ; "C11" ; Cc1cn2cc(-c3cc4ccn(C5CCNCC5)c(=O)c4s3)cc(F)c2n1 ; "804"]

To a solution of tert-butyl 4-(2-(8-fluoro-2-methylimidazo[1,2-a]pyridin-6-yl)-7-oxothieno[2,3-c]pyridin-6(7H)-yl)piperidine-1-carboxylate (217 mg, 0.45 mmol) in MeOH (10 mL) was added 4 M HCl solution in dioxane (5.0 mL, 20 mmol). The reaction mixture was stirred at rt for 2 h. The volatiles were evaporated under reduced pressure. A saturated solution of NaHCO₃ (20 mL), KOH (0.1M, 10 mL) and DCM (30 mL) were added, and the layers were separated. The aqueous layer was extracted with DCM (3×30 mL). Then, the aqueous layer was extracted with 15% iPrOH in CHCl₃ (5×100 mL). The organic layers were combined, dried over Na₂SO₄, filtered and concentrated under reduced pressure. The crude product was purified by column chromatography on silica gel using a gradient of 0-15% MeOH:Et₃N (2:1 ratio) in DCM. The fractions containing product were collected and concentrated under reduced pressure. Water (10 mL) was added, and the mixture was sonicated. The suspended material was collected by filtration and dried under vacuum to afford 2-(8-fluoro-2-methylimidazo[1,2-a]pyridin-6-yl)-6-(piperidin-4-yl)thieno[2,3-c]pyridin-7(6H)-one (132 mg, 77%) as a solid. LCMS (ES, m/z): 383.1 [M+H]⁺. ¹H NMR (DMSO-d₆, 400 MHz): δ_{H} 8.95 (1H, s), 7.86 (1H, s), 7.77 (1H, s), 7.63 (2H, d, J=9.5 Hz), 6.76 (1H, d, J=7.2 Hz), 4.85 (1H, br s), 3.08 (2H, d, J=12.1 Hz), 2.61 (2H, t, J=11.6 Hz), 2.37 (3H, s), 1.71-1.76 (4H, m).

### Synthesis of Compound 808

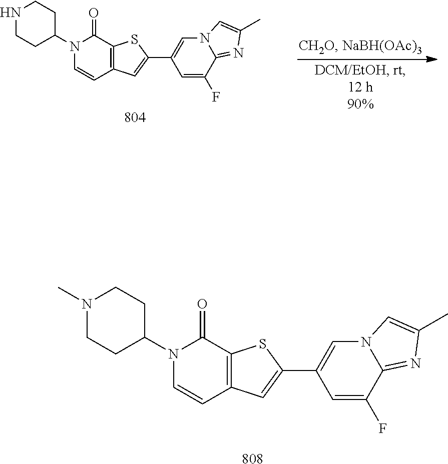

To a solution of 2-(8-fluoro-2-methylimidazo[1,2-a]pyridin-6-yl)-6-(piperidin-4-yl)thieno[2,3-c]pyridin-7(6H)-one (71 mg, 0.185 mmol) in a mixture of DCM (5.0 mL) and EtOH (2.0 mL) were sequentially added formaldehyde (37% in H₂O, 92 μL, 0.93 mmol) and NaBH(OAc)₃ (134 mg, 0.63 mmol). The reaction mixture was stirred at rt for 12 h. The volatiles were evaporated under reduced pressure. Water (10 mL) was added and the mixture was neutralized with a saturated solution of NaHCO₃. DCM (20 mL) was added and the layers were separated. The aqueous layer was extracted with DCM (3×20 mL). The organic layers were combined, dried over Na₂SO₄, filtered and concentrated under reduced pressure. MeCN (10 mL) was added and then the mixture was sonicated. The suspended material was collected by filtration, washed with MeCN (10 mL) and dried under vacuum to afford 2-(8-fluoro-2-methylimidazo[1,2-a]pyridin-6-yl)-6-(1-methylpiperidin-4-yl)thieno[2,3-c]pyridin-7(6H)-one (66 mg, 90%) as a solid. LCMS (ES, m/z): 397.1 [M+H]⁺. 1H NMR (DMSO-d₆, 400 MHz): δ_{H} 8.96 (1H, s), 7.86 (1H, s), 7.78 (1H, s), 7.64 (2H, s), 6.77 (1H, d, J=7.0 Hz), 4.81 (1H, br s), 3.03 (2H, br s), 2.35 (8H, m), 2.01 (2H, m), 1.79 (2H, br s).

### Example 47: Synthesis of Compounds 105 and 107

### Synthesis of Intermediate C12

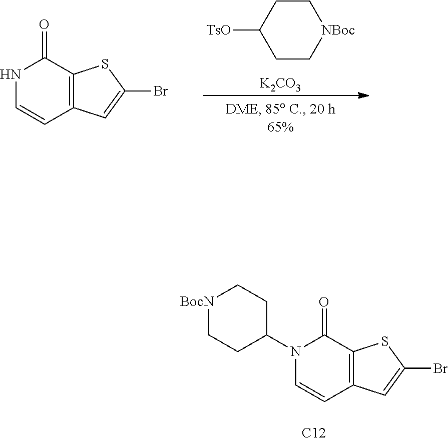

A mixture of 2-bromothieno[2,3-c]pyridin-7(6H)-one 1 (1.2 g, 5.2 mmol), tert-butyl 4-(tosyloxy)piperidine-1-carboxylate (5.5 g, 15.6 mmol) and K₂CO₃ (1.4 g, 10 mmol) in DME (42 mL) was heated to 85° C. for 20 h and then cooled to rt. The reaction mixture was filtered, and the volatiles were evaporated under reduced pressure. Water (25 mL) and DCM (30 mL) were added, and the layers were separated. The aqueous layer was extracted with (3×30 mL). The organic layers were combined, dried over Na₂SO₄, filtered and concentrated under reduced pressure. The crude product was purified by column chromatography on silica gel using a gradient of 0-50% EtOAc in hexane to afford tert-butyl 4-(2-bromo-7-oxothieno[2,3-c]pyridin-6(7H)-yl)piperidine-1-carboxylate (1.4 g, 65%) as a solid. LCMS (ES, m/z): 435.0 [M+Na]⁺.

### Synthesis of Intermediate C13

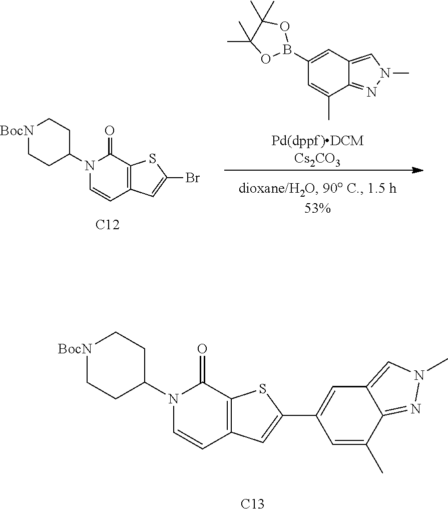

A mixture of tert-butyl 4-(2-bromo-7-oxothieno[2,3-c]pyridin-6(7H)-yl)piperidine-1-carboxylate (200 mg, 0.484 mmol), 2,7-dimethyl-5-(4,4,5,5-tetramethyl-1,3,2-dioxaborolan-2-yl)-2H-indazole (198 mg, 0.73 mmol), Cs₂CO₃ (315 mg, 0.97 mmol) and Pd(dppf)Cl₂·DCM (40 mg, 0.048 mmol) in a mixture of dioxane (7.5 mL) and H₂O (0.2 mL) was heated to 90° C. for 1.5 h and then cooled to rt. The reaction mixture was filtered over Celite using 10% MeOH in DCM as eluent. The volatiles were evaporated under reduced pressure. Water (20 mL) and DCM (20 mL) were added, and the layers were separated. The aqueous layer was extracted with DCM (3×20 mL). The organic layers were combined, dried over Na₂SO₄, filtered and concentrated under reduced pressure. The crude product was purified by column chromatography on silica gel using a gradient of 0-10% MeOH in EtOAc to afford tert-butyl 4-(2-(2,7-dimethyl-2H-indazol-5-yl)-7-oxothieno[2,3-c]pyridin-6(7H)-yl)piperidine-1-carboxylate (123 mg, 53%) as a solid. LCMS (ES, m/z): 479.2 [M+H]⁺.

### Synthesis of Compound 805

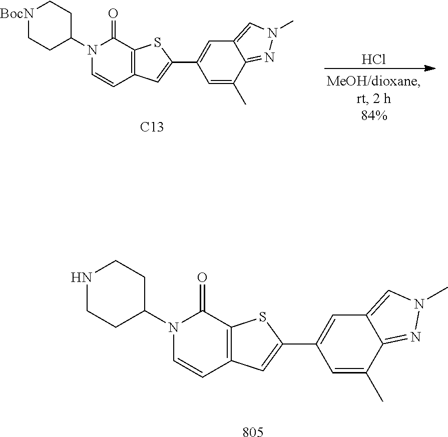 [SMILES: Cc1cc(-c2cc3ccn(C4CCN(C(=O)OC(C)(C)C)CC4)c(=O)c3s2)cc2cn(C)nc12 ; Cl ; "C13" ; Cc1cc(-c2cc3ccn(C4CCNCC4)c(=O)c3s2)cc2cn(C)nc12 ; "805"]

To a solution of tert-butyl 4-(2-(2,7-dimethyl-2H-indazol-5-yl)-7-oxothieno[2,3-c]pyridin-6(7H)-yl)piperidine-1-carboxylate (123 mg, 0.26 mmol) in MeOH (15 mL) was added 4 M HCl solution in dioxane (5.5 mL, 22 mmol). The reaction mixture was stirred at rt for 2 h. The volatiles were evaporated under reduced pressure. A saturated solution of NaHCO₃ (20 mL), KOH (0.1 M, 10 mL) and DCM (30 mL) were added, and the layers were separated. The aqueous layer was extracted with DCM (4×30 mL). The organic layers were combined, dried over sodium sulfate, filtered and concentrated under reduced pressure. The crude product was purified by column chromatography on silica gel using a gradient of 0-15% MeOH:Et₃N (2:1 ratio) in DCM. The fractions containing product were collected and concentrated under reduced pressure. Water (10 mL) was added, and the mixture was sonicated. The suspended material was collected by filtration and dried under vacuum to afford 2-(2,7-dimethyl-2H-indazol-5-yl)-6-(piperidin-4-yl)thieno[2,3-c]pyridin-7(6H)-one (82 mg, 84%) as a solid. LCMS (ES, m/z): 379.1 [M+H]⁺. ¹H NMR (DMSO-d₆, 400 MHz): δ_{H} 8.41 (1H, s), 7.98 (1H, s), 7.68 (1H, s), 7.60 (1H, d, J=7.3 Hz), 7.47 (1H, s), 6.75 (1H, d, J=7.2 Hz), 4.85 (1H, m), 4.19 (3H, s), 3.07 (2H, d, J=12.2 Hz), 2.56-2.64 (5H, m), 1.71-1.77 (4H, m).

### Synthesis of Compound 807

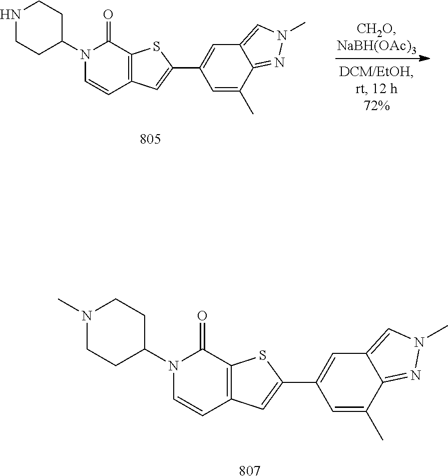

To a solution of 2-(2,7-dimethyl-2H-indazol-5-yl)-6-(piperidin-4-yl)thieno[2,3-c]pyridin-7(6H)-one (40 mg, 0.106 mmol) in a mixture of DCM (5.0 mL) and EtOH (2.0 mL) were sequentially added formaldehyde (37% in H₂O, 53 μL, 0.53 mmol) and NaBH(OAc)₃ (134 mg, 0.63 mmol). The reaction mixture was stirred at rt for 12 h. The volatiles were evaporated under reduced pressure. Water (10 mL) was added and the mixture was neutralized with a saturated solution of NaHCO₃. DCM (30 mL) was added and the layers were separated. The aqueous layer was extracted with DCM (3×30 mL). The organic layers were combined, dried over Na₂SO₄, filtered and concentrated under reduced pressure. The crude product was purified by column chromatography on silica gel using a gradient of 0-20% MeOH in DCM. The fractions containing product were collected and concentrated under reduced pressure to afford 2-(2,7-dimethyl-2H-indazol-5-yl)-6-(1-methylpiperidin-4-yl)thieno[2,3-c]pyridin-7(6H)-one (30 mg, 72%) as a solid. LCMS (ES, m/z): 393.2 [M+H]⁺. ¹H NMR (DMSO-d₆, 400 MHz): δ_{H} 8.41 (1H, s), 7.98 (1H, s), 7.68 (1H, s), 7.63 (1H, d, J=7.3 Hz), 7.47 (1H, s), 6.74 (1H, d, J=7.2 Hz), 4.77 (1H, t, J=11.6 Hz), 4.19 (3H, s), 2.92 (2H, d, J=10.8 Hz), 2.56 (3H, s), 2.23 (3H, s), 2.07 (2H, m), 1.89-1.98 (2H, m), 1.73 (2H, d, J=11.3 Hz).

### Example 48: Synthesis of Compound 806

### Synthesis of Intermediate C14

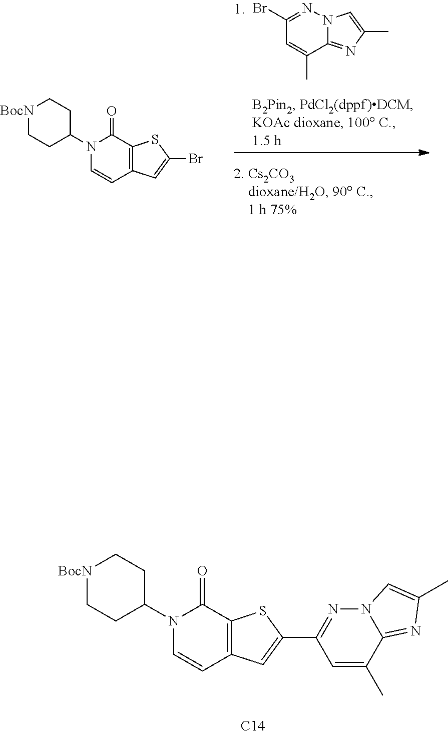 [SMILES: Cc1cn2nc(Br)cc(C)c2n1 ; CC(C)(C)OC(=O)N1CCC(n2ccc3cc(Br)sc3c2=O)CC1 ; Cc1cn2nc(-c3cc4ccn(C5CCN(C(=O)OC(C)(C)C)CC5)c(=O)c4s3)cc(C)c2n1 ; "C14"]

A mixture of 6-bromo-2,8-dimethylimidazo[1,2-b]pyridazine 1 (178 mg, 0.786 mmol), Bis(pinacolato)diboron (200 mg, 0.786 mmol), PdCl₂(dppf).DCM (44 mg, 0.060 mmol), and KOAc (180 mg, 1.84 mmol) in dioxane (4.0 mL) was heated to 100° C. for 1.5 h. A solution of tert-butyl 4-(2-(2,8-dimethylimidazo[1,2-b]pyridazin-6-yl)-7-oxothieno[2,3-c]pyridin-6(7H)-yl)piperidine-1-carboxylate (250 mg, 0.605 mmol) in dioxane (4.0 mL), Cs₂CO₃ (591 mg, 1.81 mmol) and H₂O (0.8 mL) were sequentially added under argon. The reaction mixture was heated at 90° C. for 1 h and then cooled to rt. The reaction mixture was filtered over Celite using 20% MeOH in DCM as eluent. The volatiles were evaporated under reduced pressure. Water (20 mL) and DCM (20 mL) were added, and the layers were separated. The aqueous layer was extracted with DCM (3×20 mL). The organic layers were combined, dried over Na₂SO₄, filtered and concentrated under reduced pressure. The crude material was purified by column chromatography on silica gel using a gradient of 0-100% EtOAc in hexane to afford tert-butyl 4-(2-(2,8-dimethylimidazo[1,2-b]pyridazin-6-yl)-7-oxothieno[2,3-c]pyridin-6(7H)-yl)piperidine-1-carboxylate (226 mg, 78%) as a solid. LCMS (ES, m/z): 480.2 [M+H]⁺.

### Synthesis of Compound 806

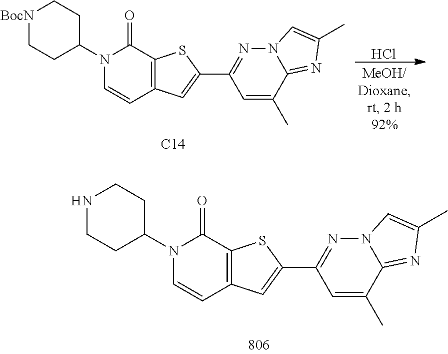 [SMILES: Cc1cn2nc(-c3cc4ccn(C5CCN(C(=O)OC(C)(C)C)CC5)c(=O)c4s3)cc(C)c2n1 ; Cl ; "C14" ; Cc1cn2nc(-c3cc4ccn(C5CCNCC5)c(=O)c4s3)cc(C)c2n1]

To a solution of tert-butyl 4-(2-(2,8-dimethylimidazo[1,2-b]pyridazin-6-yl)-7-oxothieno[2,3-c]pyridin-6(7H)-yl)piperidine-1-carboxylate 3 (226 mg, 0.471 mmol) in MeOH (10 mL) was added 4 M HCl solution in dioxane (5.2 mL, 21 mmol). The reaction mixture was stirred at rt for 2 h. The volatiles were evaporated under reduced pressure. A saturated solution of NaHCO₃ (20 mL), KOH (0.1 M, 10 mL) and DCM (30 mL) were added, and the layers were separated. The aqueous layer was extracted with DCM (3×30 mL). Then, the aqueous layer was extracted with 15% iPrOH in CHCl₃ (5×50 mL). The organic layers were combined, dried over Na₂SO₄, filtered and concentrated under reduced pressure. The crude product was purified by column chromatography on silica gel using a gradient of 0-15% MeOH:Et₃N (2:1 ratio) in DCM.

The fractions containing product were collected and concentrated under reduced pressure. Water (10 mL) was added, and the mixture was sonicated. The suspended material was collected by filtration, washed with water (10 mL) and dried under vacuum to afford 2-(2,8-dimethylimidazo[1,2-b]pyridazin-6-yl)-6-(piperidin-4-yl)thieno[2,3-c]pyridin-7(6H)-one (164 mg, 92%) as a solid. LCMS (ES, m/z): 380.1 [M+H]⁺. ¹H NMR (CHCl₃-d, 400 MHz): δ_{H} 7.77 (1H, s), 7.66 (1H, s), 7.29 (2H, m), 6.68 (1H, d, J=7.2 Hz), 5.15 (1H, t, J=11.6 Hz), 3.24 (2H, d, J=11.9 Hz), 2.87 (2H, t, J=11.9 Hz), 2.71 (3H, s), 2.53 (3H, s), 1.96 (2H, d, J=11.9 Hz), 1.77 (2H, m).

### Synthesis of Compound 807

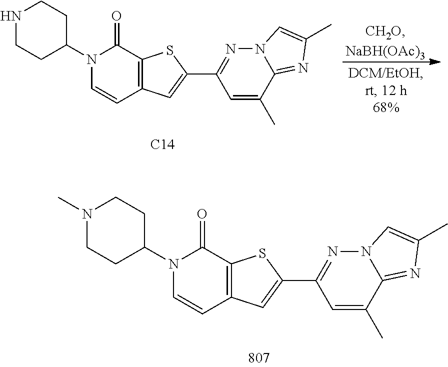

To a solution of 2-(2,8-dimethylimidazo[1,2-b]pyridazin-6-yl)-6-(piperidin-4-yl)thieno[2,3-c]pyridin-7(6H)-one (50 mg, 0.132 mmol) in a mixture of DCM (5.0 mL) and EtOH (2.0 mL) were sequentially added formaldehyde (37% in H₂O, 66 μL, 0.66 mmol mmol) and NaBH(OAc)₃ (168 mg, 0.79 mmol). The reaction mixture was stirred at rt for 12 h. The volatiles were evaporated under reduced pressure. Water (5.0 mL) was added and the mixture was neutralized with a saturated solution of NaHCO₃. DCM (10 mL) was added and the layers were separated. The aqueous layer was extracted with DCM (3×10 mL). The organic layers were combined, dried over Na₂SO₄, filtered and concentrated under reduced pressure. Water (10 mL) was added and the mixture was sonicated. The suspended material was collected by filtration, washed with water (10 mL) and dried under vacuum to afford 2-(2,8-dimethylimidazo[1,2-b]pyridazin-6-yl)-6-(1-methylpiperidin-4-yl)thieno[2,3-c]pyridin-7(6H)-one (35 mg, 68%) as a solid. LCMS (ES, m/z): 394.1 [M+H]⁺. 1H NMR (DMSO-d₆, 400 MHz): δ_{H} 8.11 (1H, s), 8.09 (1H, s), 7.77 (1H, s), 7.69 (1H, d, J=7.3 Hz), 6.80 (1H, d, J=7.2 Hz), 4.76 (1H, t, J=11.5 Hz), 2.92 (2H, d, J=10.8 Hz), 2.60 (3H, s), 2.40 (3H, s), 2.22 (3H, s), 1.91-2.09 (4H, m), 1.74 (2H, d, J=11.3 Hz).

### Example 49: Synthesis of Compound 812

### Synthesis of Intermediate C15

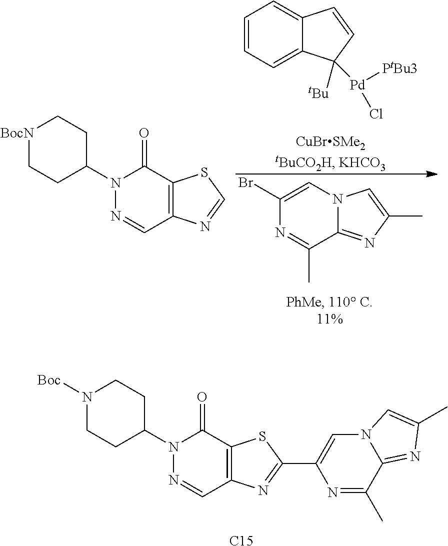

A flame-dried 20 mL sealed tube was charged with tert-butyl 4-(7-oxothiazolo[4,5-d]pyridazin-6(7H)-yl)piperidine-1-carboxylate 164 mg, 0.487 mmol), 6-bromo-2,8-dimethylimidazo[1,2-a]pyrazine (100 mg, 0.442 mmol), pivalic acid (18 mg, 0.18 mmol), potassium bicarbonate (90 mg, 0.88 mmol), and CuBr SMe₂ (18 mg, 89 μmol). The mixture was dissolved in dry toluene (4.4 mL) and argon was bubbled through it for 10 minutes. To this solution was added chloro(1-tert-butyl-1H-inden-1-yl)(tri-tert-butylphosphine)palladium(II) (11 mg, 22 μmol) under flow of argon. The tube was sealed, and the reaction mixture was heated at 110° C. overnight. Upon completion, the mixture was diluted with EtOAc (25 mL) and filtered through a pad of celite using additional EtOAc (15 mL) to wash the filter cake. The filtrate was washed with saturated NaHCO₃ (20 mL) and brine (2×20 mL). The organic phase was dried over Na₂SO₄, filtered, and concentrated in vacuo. The residue was purified by flash chromatography on a 12 g silica gel column using a gradient of 0-100% DCM in EtOAc. Selected fractions were combined and evaporated under reduced pressure to afford t tert-butyl 4-(2-(2,8-dimethylimidazo[1,2-a]pyrazin-6-yl)-7-oxothiazolo[4,5-d]pyridazin-6(7H)-yl)piperidine-1-carboxylate (23 mg, 11%) as a solid. LCMS (ES, m/z): 482.2 [M+H]⁺.

### Synthesis of Compound 812

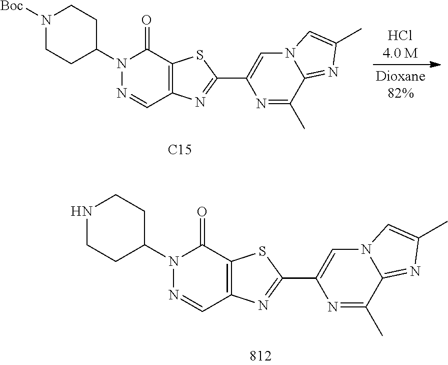 [SMILES: Cc1cn2cc(-c3nc4cnn(C5CCN(C(=O)OC(C)(C)C)CC5)c(=O)c4s3)nc(C)c2n1 ; "C15" ; Cc1cn2cc(-c3nc4cnn(C5CCNCC5)c(=O)c4s3)nc(C)c2n1]

A 20 mL vial was charged with tert-butyl 4-(2-(2,8-dimethylimidazo[1,2-a]pyrazin-6-yl)-7-oxothiazolo[4,5-d]pyridazin-6(7H)-yl)piperidine-1-carboxylate 3 (23 mg, 48 μmol). To this was added HCl 4.0 M in dioxane (1.2 mL). The mixture was stirred vigorously at room temperature for 90 minutes. Upon completion, the reaction mixture was concentrated to dryness under reduced pressure. The residue was partitioned between DCM (20 mL) and 0.25 M NaOH (20 mL) and stirred to neutralize. The phases were separated, and the aqueous phase was washed with DCM (2×20 mL). The organic phases were combined, dried over Na₂SO₄, filtered, and concentrated in vacuo. The residue was purified by flash chromatography on an 8 g neutral alumina column using a gradient of 0-10% MeOH in DCM. Selected fractions were combined and evaporated under reduced pressure to afford 2-(2,8-dimethylimidazo[1,2-a]pyrazin-6-yl)-6-(piperidin-4-yl)thiazolo[4,5-d]pyridazin-7(6H)-one (15 mg, 82%) as a solid. LCMS (ES, m/z): 382.1 [M+H]⁺. ¹H NMR (CDCl₃, 300 MHz): δ 8.95 (1H, s), 8.50 (1H, s), 7.57 (1H, s), 5.19 (1H, m), 3.28 (2H, d, J=12.8 Hz), 2.96 (3H, s), 2.86 (2H, t, J=12.2 Hz), 2.57 (3H, s), 1.94-2.07 (4H, m), 1.28 (1H, s).

### Example 50: Synthesis of Compound 800

### Synthesis of Intermediate C16

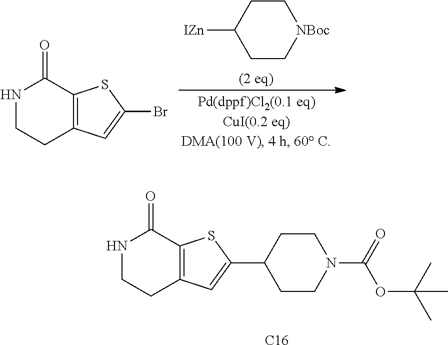

A solution of 2-bromo-4H,5H,6H-thieno[2,3-c]pyridin-7-one (300 mg, 1.267 mmol, 1.00 equiv) in DMA (30 mL) was treated with Pd(dppf)Cl₂ (103 mg, 0.127 mmol, 0.10 equiv) and CuI (48 mg, 0.253 mmol, 0.20 equiv) for 4 h at 60 degrees C. under nitrogen atmosphere followed by the addition of [1-(tert-butoxycarbonyl)piperidin-4-yl](iodo)zinc (2.53 mL, 2.534 mmol, 2.00 equiv)(1 M in DMA) dropwise at 60 degrees C. The resulting mixture was stirred for 4 h at 60 degrees C. under nitrogen atmosphere. The resulting mixture was filtered, the filter cake was washed with ethyl acetate (3×50 mL). The combined organic layers were washed with brine (3×100 mL), dried over anhydrous Na₂SO₄. After filtration, the filtrate was concentrated under reduced pressure to afford tert-butyl 4-[7-oxo-4H,5H,6H-thieno[2,3-c]pyridin-2-yl]piperidine-1-carboxylate (250 mg, 57%) as a solid. LCMS (ES, m/z): 337 [M+H]⁺

### Synthesis of Intermediate C17

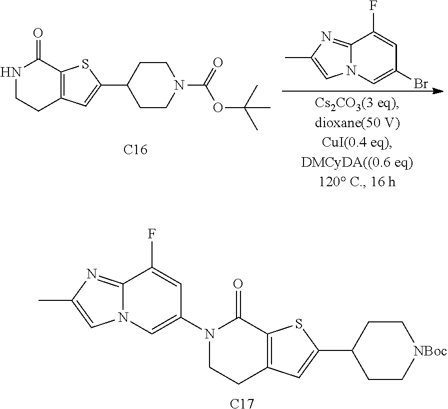

A solution of tert-butyl 4-[7-oxo-4H,5H,6H-thieno[2,3-c]pyridin-2-yl]piperidine-1-carboxylate (250 mg, 0.728 mmol, 1.00 equiv), 6-bromo-8-fluoro-2-methylimidazo[1,2-a]pyridine (200 mg, 0.874 mmol, 1.20 equiv), CuI (55 mg, 0.291 mmol, 0.40 equiv), (1R,2R)-1-N,2-N-dimethylcyclohexane-1,2-diamine (62 mg, 0.437 mmol, 0.60 equiv) and Cs₂CO₃ (712 mg, 2.184 mmol, 3.00 equiv) in 1,4-dioxane (12.5 mL) was stirred for 16 h at 120 degrees C. under nitrogen atmosphere. The mixture was allowed to cool down to 25 degrees C. The resulting mixture was filtered, the filter cake was washed with ethyl acetate (3×50 mL). The combined organic layers were washed with water (2×100 mL), dried over anhydrous Na₂SO₄. After filtration, the filtrate was concentrated under reduced pressure. The residue was purified by silica gel column chromatography, eluted with PE/EA (1:1) to afford tert-butyl 4-(6-[8-fluoro-2-methylimidazo[1,2-a]pyridin-6-yl]-7-oxo-4H,5H-thieno[2,3-c]pyridin-2-yl)piperidine-1-carboxylate (70 mg, 19%) as a solid. LCMS (ES, m/z): 485 [M+H]⁺

### Synthesis of Compound 800

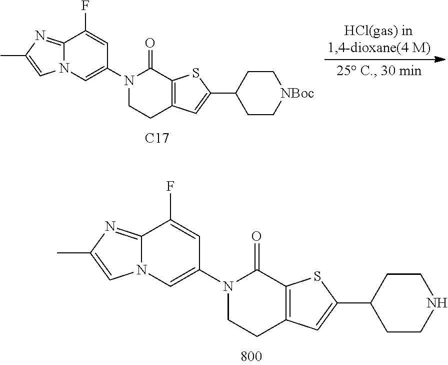 [SMILES: Cc1cn2cc(N3CCc4cc(C5CCN(C(=O)OC(C)(C)C)CC5)sc4C3=O)cc(F)c2n1 ; "C17" ; Cc1cn2cc(N3CCc4cc(C5CCNCC5)sc4C3=O)cc(F)c2n1]

A solution of tert-butyl 4-(6-[8-fluoro-2-methylimidazo[1,2-a]pyridin-6-yl]-7-oxo-4H,5H-thieno[2,3-c]pyridin-2-yl)piperidine-1-carboxylate (30 mg, 0.061 mmol, 1.00 equiv) in HCl(gas) in 1,4-dioxane (3 mL) was stirred for 30 min at 25 degrees C. The resulting mixture was concentrated under reduced pressure. The crude product was purified by Prep-HPLC with the following conditions: Column, Xselect CSH OBD Column 30×150 mm 5 um; mobile phase, water (10 MMOL/L NH₄HCO₃) and ACN (5% PhaseB up to 55% in 8 min); Detector, UV 220 nm to afford 6-[8-fluoro-2-methylimidazo[1,2-a]pyridin-6-yl]-2-(piperidin-4-yl)-4H,5H-thieno[2,3-c]pyridin-7-one (13.1 mg, 55%) as a solid. LCMS (ES, m/z): 385 [M+H]⁺¹H NMR (400 MHz, DMSO-d₆) δ 8.50 (d, J=1.7 Hz, 1H), 7.82 (dd, J=3.1, 1.0 Hz, 1H), 7.26 (dd, J=12.3, 1.7 Hz, 1H), 6.92 (d, J=0.7 Hz, 1H), 3.99 (t, J=6.8 Hz, 2H), 3.06-2.87 (m, 4H), 2.99 (s, 1H), 2.63-2.52 (m, 2H), 2.37 (d, J=0.9 Hz, 3H), 1.88 (d, J=11.9 Hz, 2H), 1.51 (dd, J=12.1, 3.9 Hz, 1H), 1.45 (dd, J=12.0, 3.9 Hz, 1H).

### Example 51: Synthesis of Compound 801

### Synthesis of Intermediate C18

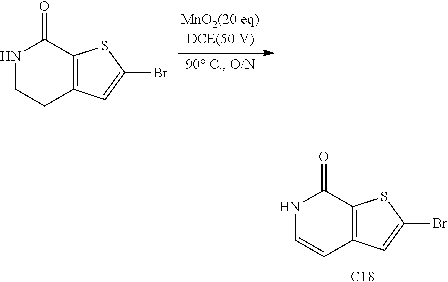 [SMILES: O=C1NCCc2cc(Br)sc21 ; O=c1[nH]ccc2cc(Br)sc12 ; "C18"]

A mixture of 2-bromo-4H,5H,6H-thieno[2,3-c]pyridin-7-one (350 mg, 1.478 mmol, 1.00 equiv) and MnO₂ (2.57 g, 29.562 mmol, 20.00 equiv) in DCE (17.50 mL) was stirred for overnight at 90 degrees C. The mixture was allowed to cool down to room temperature. The resulting mixture was filtered, the filter cake was washed with DCE (2×20 mL). The filtrate was concentrated under reduced pressure. The residue was purified by silica gel column chromatography, eluted with PE/EA (1:1) to afford 2-bromo-6H-thieno[2,3-c]pyridin-7-one (150 mg, 43%) as a solid. LCMS (ES, m/z): 230/232 [M+H]⁺

### Synthesis of Intermediate C19

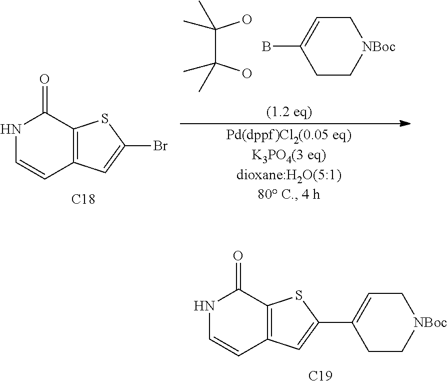

A solution of 2-bromo-6H-thieno[2,3-c]pyridin-7-one (130 mg, 0.554 mmol, 1.00 equiv), tert-butyl 4-(4,4,5,5-tetramethyl-1,3,2-dioxaborolan-2-yl)-3,6-dihydro-2H-pyridine-1-carboxylate (205 mg, 0.665 mmol, 1.20 equiv), Pd(dppf)Cl₂ (20 mg, 0.028 mmol, 0.05 equiv) and K₃PO₄ (353 mg, 1.661 mmol, 3.00 equiv) in 1,4-dioxane (5.00 mL) and H₂O (1.00 mL) was stirred for 4 h at 80 degrees C. under nitrogen atmosphere. The mixture was allowed to cool down to room temperature. The resulting mixture was concentrated under reduced pressure. The residue was purified by silica gel column chromatography, eluted with PE/EA (1:1) to afford tert-butyl 4-[7-oxo-6H-thieno[2,3-c]pyridin-2-yl]-3,6-dihydro-2H-pyridine-1-carboxylate (95 mg, 50.58%) as a solid. LCMS (ES, m/z): 333 [M+H]⁺

### Synthesis of Intermediate C20

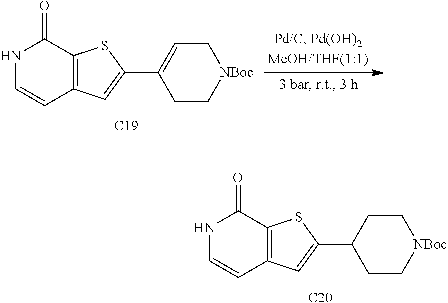 [SMILES: CC(C)(C)OC(=O)N1CC=C(c2cc3cc[nH]c(=O)c3s2)CC1 ; "C19" ; CC(C)(C)OC(=O)N1CCC(c2cc3cc[nH]c(=O)c3s2)CC1 ; "C20"]

A mixture of tert-butyl 4-[7-oxo-6H-thieno[2,3-c]pyridin-2-yl]-3,6-dihydro-2H-pyridine-1-carboxylate (90 mg, 0.265 mmol, 1.00 equiv), Pd/C (45 mg, 0.042 mmol, 0.16 equiv, 10%) and Pd(OH)₂/C (45 mg, 0.032 mmol, 0.12 equiv, 10%) in MeOH (2.00 mL) and THF (2.00 mL) was stirred for 3 h at room temperature at 3 bar under hydrogen atmosphere. The resulting mixture was filtered, the filter cake was washed with THE (2×10 mL). The filtrate was concentrated under reduced pressure to afford tert-butyl 4-[7-oxo-6H-thieno[2,3-c]pyridin-2-yl]piperidine-1-carboxylate (80 mg, 88%) as a solid. LCMS (ES, m/z): 335 [M+H]⁺

### Synthesis of Intermediate C21

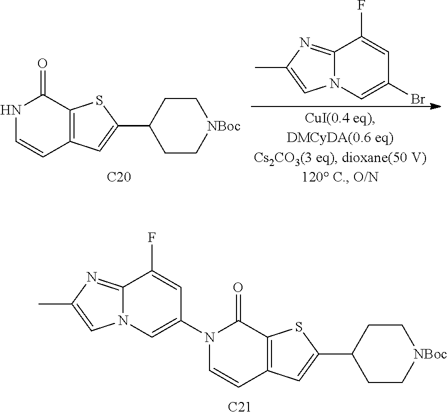 [SMILES: CC(C)(C)OC(=O)N1CCC(c2cc3cc[nH]c(=O)c3s2)CC1 ; Cc1cn2cc(Br)cc(F)c2n1 ; "C20" ; Cc1cn2cc(-n3ccc4cc(C5CCN(C(=O)OC(C)(C)C)CC5)sc4c3=O)cc(F)c2n1 ; "C21"]

A solution of tert-butyl 4-[7-oxo-6H-thieno[2,3-c]pyridin-2-yl]piperidine-1-carboxylate (80 mg, 0.234 mmol, 1.00 equiv), 6-bromo-8-fluoro-2-methylimidazo[1,2-a]pyridine (64 mg, 0.281 mmol, 1.20 equiv), CuI (18 mg, 0.094 mmol, 0.40 equiv), (1R,2R)-1-N,2-N-dimethylcyclohexane-1,2-diamine (20 mg, 0.140 mmol, 0.60 equiv) and Cs₂CO₃ (229 mg, 0.702 mmol, 3.00 equiv) in 1,4-dioxane (4.00 mL) was stirred for overnight at 120 degrees C. under nitrogen atmosphere. The mixture was allowed to cool down to room temperature. The resulting mixture was filtered, the filter cake was washed with 1,4-dioxane (2×10 mL). The filtrate was concentrated under reduced pressure. The residue was purified by silica gel column chromatography, eluted with PE/EA (1:1) to afford tert-butyl 4-(6-[8-fluoro-2-methylimidazo[1,2-a]pyridin-6-yl]-7-oxothieno[2,3-c]pyridin-2-yl)piperidine-1-carboxylate (50 mg, 43%) as a solid. LCMS (ES, m/z): 483 [M+H]⁺

### Synthesis of Compound 801

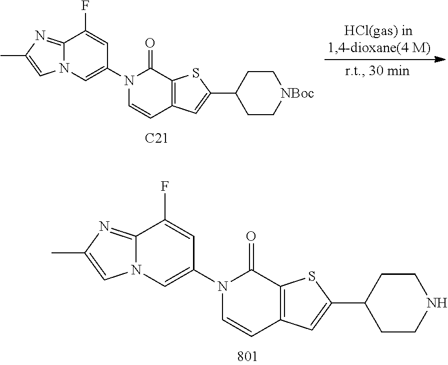 [SMILES: Cc1cn2cc(-n3ccc4cc(C5CCN(C(=O)OC(C)(C)C)CC5)sc4c3=O)cc(F)c2n1 ; "C21" ; Cc1cn2cc(-n3ccc4cc(C5CCNCC5)sc4c3=O)cc(F)c2n1]

A solution of tert-butyl 4-(6-[8-fluoro-2-methylimidazo[1,2-a]pyridin-6-yl]-7-oxothieno[2,3-c]pyridin-2-yl)piperidine-1-carboxylate (50 mg, 0.102 mmol, 1.00 equiv) in HCl(gas) in 1,4-dioxane (2.00 mL) was stirred for 30 min at room temperature. The resulting mixture was concentrated under reduced pressure. The crude product was purified by Prep-HPLC with the following conditions: Column, YMC-Actus Triart C18, 30 mm×150 mm, Sum; mobile phase, water (10MMOL/L NH₄HCO₃) and ACN (5% PhaseB up to 40% in 8 min); Detector, UV 220 nm to afford 6-[8-fluoro-2-methylimidazo[1,2-a]pyridin-6-yl]-2-(piperidin-4-yl)thieno[2,3-c]pyridin-7-one(21.1 mg, 53%) as a solid. LCMS (ES, m/z): 383 [M+H]^{+ 1}H NMR (400 MHz, DMSO-d₆) δ 8.70 (d, J=1.7 Hz, 1H), 7.89 (d, J=3.0 Hz, 1H), 7.62 (d, J=7.1 Hz, 1H), 7.37 (dd, J=11.6, 1.7 Hz, 1H), 7.23 (s, 1H), 6.80 (d, J=7.1 Hz, 1H), 4.09 (s, 1H), 3.04 (d, J=11.4 Hz, 2H), 2.85 (s, 1H), 2.63 (s, 1H), 2.40 (s, 3H), 1.96 (d, J=12.5 Hz, 2H), 1.56 (qd, J=12.2, 3.9 Hz, 2H).

### Example 52: Synthesis of Compound 272

### Synthesis of Intermediate C22

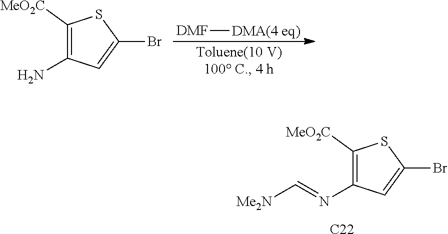

A solution of methyl 3-amino-5-bromothiophene-2-carboxylate (5 g, 20.756 mmol, 1.00 equiv) and DMF-DMA (9.89 g, 83.024 mmol, 4 equiv) in toluene (50 mL) was stirred for 4 h at 100 degrees C. The mixture was allowed to cool down to room temperature. The resulting mixture was concentrated under vacuum to afford methyl 5-bromo-3-[(E)-[(dimethylamino)methylidene]amino]thiophene-2-carboxylate (5.1 g, 76%) as a solid. LCMS (ES, m/z):291 [M+H]⁺

### Synthesis of Intermediate C23

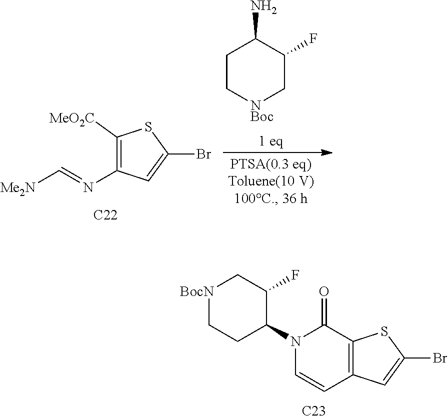 [SMILES: CC(C)(C)OC(=O)N1CC[C@@H](N)[C@H](F)C1 ; COC(=O)c1sc(Br)cc1/N=C/N(C)C ; "C22" ; CC(C)(C)OC(=O)N1CC[C@H](n2ccc3cc(Br)sc3c2=O)[C@@H](F)C1 ; "C23"]

A solution of methyl 5-bromo-3-[(E)-[(dimethylamino)methylidene]amino]thiophene-2-carboxylate (300 mg, 1.010 mmol, 1.00 equiv), trans-tert-butyl-4-amino-3-fluoropiperidine-1-carboxylate (220 mg, 1.010 mmol, 1 equiv) and PTSA (52 mg, 0.303 mmol, 0.3 equiv) in toluene (10 mL) was stirred for 16 h at 100 degrees C. The mixture was allowed to cool down to room temperature. The resulting mixture was concentrated under vacuum. The residue was purified by silica gel column chromatography, eluted with PE/EA (5:1) to afford trans-tert-butyl-4-{6-bromo-4-oxothieno[3,2-d]pyrimidin-3-yl}-3-fluoropiperidine-1-carboxylate (280 mg, 63%) as a solid. LCMS (ES, m/z):432 [M+H]⁺

### Synthesis of Intermediate C24

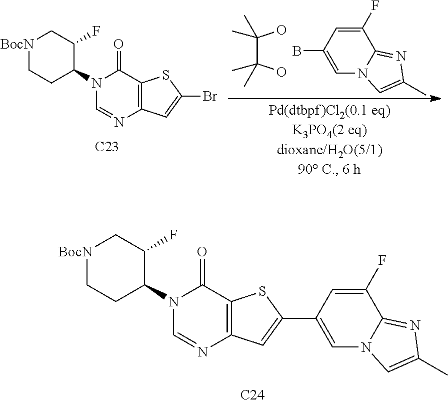

A solution of trans-tert-butyl-4-{6-bromo-4-oxothieno[3,2-d]pyrimidin-3-yl}-3-fluoropiperidine-1-carboxylate (80 mg, 0.185 mmol, 1.00 equiv) and 8-fluoro-2-methyl-6-(4,4,5,5-tetramethyl-1,3,2-dioxaborolan-2-yl)imidazo[1,2-a]pyridine (128 mg, 0.463 mmol, 2.5 equiv), K₃PO₄ (79 mg, 0.370 mmol, 2 equiv) in 1,4-dioxane (1 mL) was stirred for 6 h at 90 degrees C. under nitrogen atmosphere. The mixture was allowed to cool down to room temperature. The resulting mixture was extracted with EA (1×5 mL), dried over anhydrous Na₂SO₄. After filtration, the filtrate was concentrated under reduced pressure. The residue was purified by silica gel column chromatography, eluted with PE/EA (1:1) to afford trans-tert-butyl-3-fluoro-4-(6-{8-fluoro-2-methylimidazo[1,2-a]pyridin-6-yl}-4-oxothieno[3,2-d]pyrimidin-3-yl)piperidine-1-carboxylate (280 mg) as a solid. LCMS (ES, m/z):502 [M+H]⁺

### Synthesis of Compound 272

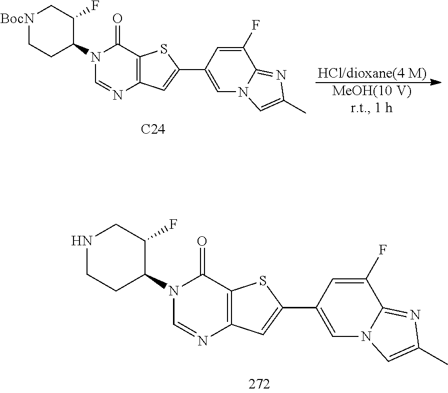 [SMILES: Cc1cn2cc(-c3cc4ncn([C@H]5CCN(C(=O)OC(C)(C)C)C[C@@H]5F)c(=O)c4s3)cc(F)c2n1 ; "C24" ; Cc1cn2cc(-c3cc4ncn([C@H]5CCNC[C@@H]5F)c(=O)c4s3)cc(F)c2n1]

A solution of trans-tert-butyl-3-fluoro-4-(6-{8-fluoro-2-methylimidazo[1,2-a]pyridin-6-yl}-4-oxothieno[3,2-d]pyrimidin-3-yl)piperidine-1-carboxylate (70 mg, 0.140 mmol, 1.00 equiv) in MeOH (1 mL) was stirred for 1 h at room temperature. The resulting mixture was concentrated under reduced pressure. The crude product was purified by Prep-HPLC with the following conditions (Column: YMC-Actus Triart C18, 30×150 mm, 5 m; Mobile Phase A: water (10 mmol/L NH₄HCO₃), Mobile Phase B: ACN; Flow rate: 60 mL/min; Gradient: 5% B to 50% B in 8 min, 50% B; Wave Length: 220 nm; RT1(min): 8.46) to afford trans-6-{8-fluoro-2-methylimidazo[1,2-a]pyridin-6-yl}-3-[3-fluoropiperidin-4-yl]thieno[3,2-d]pyrimidin-4-one (17.6 mg, 31%) as a solid.LCMS (ES, m/z):402 [M+H]^{+ 1}H NMR (400 MHz, DMSO-d₆) δ 9.04 (d, J=1.5 Hz, 1H), 8.63 (s, 1H), 7.93 (s, 1H), 7.87 (dd, J=3.2, 1.1 Hz, 1H), 7.74 (dd, J=12.1, 1.6 Hz, 1H), 5.05-5.00 (m, 1H), 4.81 (s, 1H), 3.36 (m, 2H), 2.99 (d, J=12.3 Hz, 1H), 2.48 (m, 1H), 2.39 (s, 3H), 2.12 (d, J=13.5 Hz, 1H), 1.93 (d, J=11.3 Hz, 1H).

### Example 53: Synthesis of Compound 273

### Synthesis of Intermediate C25

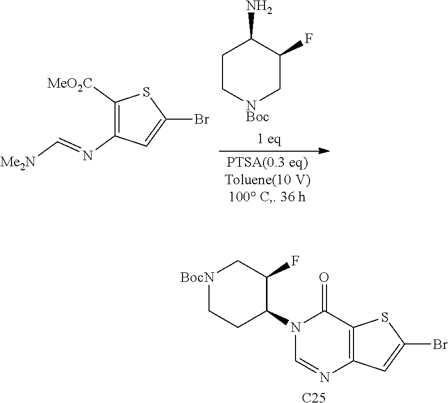

A solution of methyl 5-bromo-3-[(E)-[(dimethylamino)methylidene]amino]thiophene-2-carboxylate (300 mg, 1.030 mmol, 1.00 equiv), cis-tert-butyl-4-amino-3-fluoropiperidine-1-carboxylate (225 mg, 1.030 mmol, 1 equiv) and PTSA (53 mg, 0.303 mmol, 0.3 equiv) in toluene (10 mL) was stirred for 16 h at 100 degrees C. The mixture was allowed to cool down to room temperature. The resulting mixture was concentrated under vacuum. The residue was purified by silica gel column chromatography, eluted with PE/EA (5:1) to afford cis-tert-butyl-4-{6-bromo-4-oxothieno[3,2-d]pyrimidin-3-yl}-3-fluoropiperidine-1-carboxylate (170 mg, 37%) as a solid. LCMS (ES, m/z):432 [M+H]⁺

### Synthesis of Intermediate C26

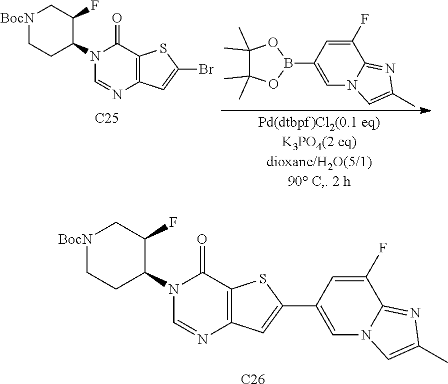

A solution of cis-tert-butyl-4-{6-bromo-4-oxothieno[3,2-d]pyrimidin-3-yl}-3-fluoropiperidine-1-carboxylate (80 mg, 0.185 mmol, 1.00 equiv) and 8-fluoro-2-methyl-6-(4,4,5,5-tetramethyl-1,3,2-dioxaborolan-2-yl)imidazo[1,2-a]pyridine (127 mg, 0.463 mmol, 2.5 equiv), K₃PO₄ (118 mg, 0.555 mmol, 3 equiv) in 1,4-dioxane (1 mL) was stirred for 2 h at 90 degrees C. under nitrogen atmosphere. The mixture was allowed to cool down to room temperature. The resulting mixture was extracted with EA (1×5 mL). dried over anhydrous Na₂SO₄. After filtration, the filtrate was concentrated under reduced pressure. The residue was purified by silica gel column chromatography, eluted with PE/EA (1:1) to afford cis-tert-butyl-3-fluoro-4-(6-{8-fluoro-2-methylimidazo[1,2-a]pyridin-6-yl}-4-oxothieno[3,2-d]pyrimidin-3-yl)piperidine-1-carboxylate (50 mg) as a solid. LCMS (ES, m/z):502 [M+H]⁺

### Synthesis Compound 273

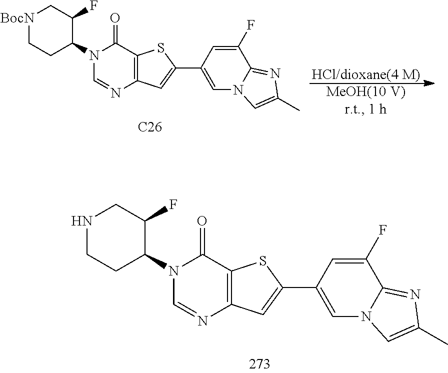 [SMILES: Cc1cn2cc(-c3cc4ncn([C@H]5CCN(C(=O)OC(C)(C)C)C[C@H]5F)c(=O)c4s3)cc(F)c2n1 ; "C26" ; Cc1cn2cc(-c3cc4ncn([C@H]5CCNC[C@H]5F)c(=O)c4s3)cc(F)c2n1]

A solution of cis-tert-butyl-3-fluoro-4-(6-{8-fluoro-2-methylimidazo[1,2-a]pyridin-6-yl}-4-oxothieno[3,2-d]pyrimidin-3-yl)piperidine-1-carboxylate (50 mg, 0.100 mmol, 1.00 equiv) in MeOH (1 mL) was stirred for 1 h at room temperature. The resulting mixture was concentrated under reduced pressure. The crude product was purified by Prep-HPLC with the following conditions (Column: XBridge Prep OBD C18 Column, 30×150 mm, 5 m; Mobile Phase A: water (10 mmol/L NH₄HCO₃), Mobile Phase B: ACN; Flow rate: 60 mL/min; Gradient: 5% B to 36% B in 8 min, 36% B; Wave Length: 220 nm; RT1(min): 7.40) to afford cis-6-{8-fluoro-2-methylimidazo[1,2-a]pyridin-6-yl}-3-[3-fluoropiperidin-4-yl]thieno[3,2-d]pyrimidin-4-one (7.6 mg, 19%) as a white solid. LCMS (ES, m/z):402 [M+H]^{+ 1}H NMR (400 MHz, DMSO-d₆) δ 9.05 (d, J=1.6 Hz, 1H), 8.39 (d, J=1.6 Hz, 1H), 7.93 (s, 1H), 7.87 (dd, J=3.2, 1.0 Hz, 1H), 7.74 (dd, J=12.1, 1.6 Hz, 1H), 5.12-5.04 (m, 1H), 5.03-4.95 (m, 1H), 3.27-3.09 (m, 1H), 2.92 (d, J=14.6 Hz, 1H), 2.82 (d, J=14.6 Hz, 1H), 2.73 (d, J=12.4 Hz, 1H), 2.41-2.37 (m, 3H), 2.33-2.25 (m, 1H), 2.14 (s, 1H), 1.72 (d, J=10.5 Hz, 1H).

### Example 54: Synthesis of Compound 274

### Synthesis of Intermediate C27

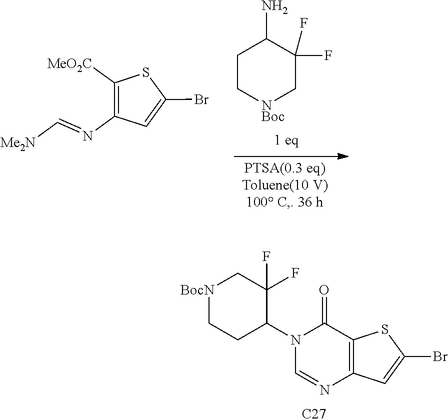

A solution of methyl 5-bromo-3-[(E)-[(dimethylamino)methylidene]amino]thiophene-2-carboxylate (300 mg, 1.030 mmol, 1.00 equiv), tert-butyl 4-amino-3,3-difluoropiperidine-1-carboxylate (243 mg, 1.030 mmol, 1 equiv) and PTSA (53 mg, 0.303 mmol, 0.3 equiv) in toluene (10 mL) was stirred for 16 h at 100 degrees C. The mixture was allowed to cool down to room temperature. The resulting mixture was concentrated under vacuum. The residue was purified by silica gel column chromatography, eluted with PE/EA (5:1) to afford tert-butyl 4-{6-bromo-4-oxothieno[3,2-d]pyrimidin-3-yl}-3,3-difluoropiperidine-1-carboxylate (70 mg, 15%) as a solid. LCMS (ES, m/z):450 [M+H]⁺

### Synthesis of Intermediate C28

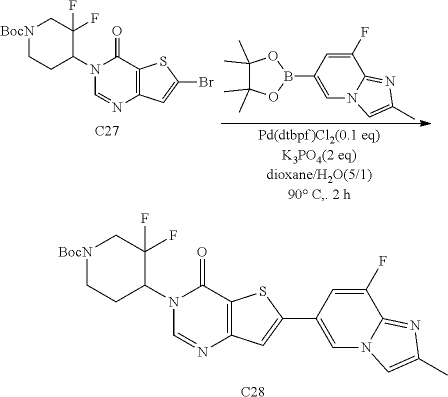

A solution of tert-butyl 4-{6-bromo-4-oxothieno[3,2-d]pyrimidin-3-yl}-3,3-difluoropiperidine-1-carboxylate (80 mg, 0.178 mmol, 1.00 equiv) and 8-fluoro-2-methyl-6-(4,4,5,5-tetramethyl-1,3,2-dioxaborolan-2-yl)imidazo[1,2-a]pyridine (123 mg, 0.445 mmol, 2.5 equiv), K₃PO₄ (113 mg, 0.534 mmol, 3 equiv) in 1,4-dioxane (1 mL) was stirred for 2 h at 90 degrees C. under nitrogen atmosphere. The mixture was allowed to cool down to room temperature. The resulting mixture was extracted with EA (1×5 mL). dried over anhydrous Na₂SO₄. After filtration, the filtrate was concentrated under reduced pressure. The residue was purified by silica gel column chromatography, eluted with PE/EA (1:1) to afford tert-butyl 3,3-difluoro-4-(6-{8-fluoro-2-methylimidazo[1,2-a]pyridin-6-yl}-4-oxothieno[3,2-d]pyrimidin-3-yl)piperidine-1-carboxylate (35 mg, 38%) as a solid. LCMS (ES, m/z):520 [M+H]⁺

### Synthesis of Compound 274

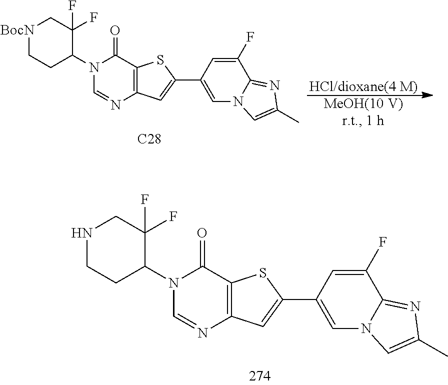 [SMILES: Cc1cn2cc(-c3cc4ncn(C5CCN(C(=O)OC(C)(C)C)CC5(F)F)c(=O)c4s3)cc(F)c2n1 ; "C28" ; Cc1cn2cc(-c3cc4ncn(C5CCNCC5(F)F)c(=O)c4s3)cc(F)c2n1]

A solution of tert-butyl 3,3-difluoro-4-(6-{8-fluoro-2-methylimidazo[1,2-a]pyridin-6-yl}-4-oxothieno[3,2-d]pyrimidin-3-yl)piperidine-1-carboxylate (35 mg, 0.067 mmol, 1.00 equiv) in MeOH (1 mL) was stirred for 1 h at room temperature. The resulting mixture was concentrated under reduced pressure. The crude product was purified by Prep-HPLC with the following conditions (Column: XBridge Prep OBD C18 Column, 30×150 mm, 5 m; Mobile Phase A: water (10 mmol/L NH₄HCO₃), Mobile Phase B: ACN; Flow rate: 60 mL/min; Gradient: 5% B to 41% B in 8 min, 41% B; Wave Length: 220 nm; RT1(min): 7.45) to afford 3-(3,3-difluoropiperidin-4-yl)-6-{8-fluoro-2-methylimidazo[1,2-a]pyridin-6-yl}thieno[3,2-d]pyrimidin-4-one (10.1 mg, 36%) as a solid. LCMS (ES, m/z):420 [M+H]^{+ 1}H NMR (400 MHz, DMSO-d₆) δ 9.05 (d, J=1.6 Hz, 1H), 8.48 (d, J=2.7 Hz, 1H), 7.94 (s, 1H), 7.87 (dd, J=3.2, 1.1 Hz, 1H), 7.74 (dd, J=12.1, 1.6 Hz, 1H), 5.51-5.36 (m, 1H), 3.23 (d, J=12.6 Hz, 1H), 3.09 (d, J=13.1 Hz, 2H), 2.78 (q, J=11.3 Hz, 1H), 2.62 (s, 1H), 2.39 (s, 3H), 2.37-2.31 (m, 1H), 2.04-1.96 (m, 1H).

### Example 55: Synthesis of Compound 276

### Synthesis of Intermediate C29

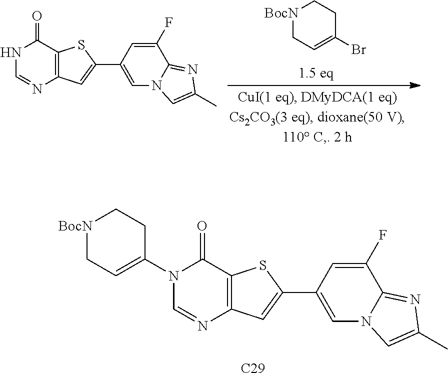 [SMILES: Cc1cn2cc(-c3cc4nc[nH]c(=O)c4s3)cc(F)c2n1 ; CC(C)(C)OC(=O)N1CC=C(Br)CC1 ; Cc1cn2cc(-c3cc4ncn(C5=CCN(C(=O)OC(C)(C)C)CC5)c(=O)c4s3)cc(F)c2n1 ; "C29"]

A solution of 6-{8-fluoro-2-methylimidazo[1,2-a]pyridin-6-yl}-3H-thieno[3,2-d]pyrimidin-4-one (360 mg, 1.199 mmol, 1.00 equiv) and tert-butyl 4-bromo-3,6-dihydro-2H-pyridine-1-carboxylate (471.38 mg, 1.799 mmol, 1.5 equiv), (CIS)-1-N,2-N-dimethylcyclohexane-1,2-diamine (170 mg, 1.199 mmol, 1 equiv), Cs₂CO₃ (1171 mg, 3.597 mmol, 3 equiv),CuI (190.25 mg, 0.999 mmol, 1 equiv) in 1,4-dioxane (3.6 mL) was stirred for 2 h at 110 degrees C. under nitrogen atmosphere. The mixture was allowed to cool down to r.t. The resulting mixture was washed with 1×5 mL of MeOH. The residue was purified by silica gel column chromatography, eluted with PE/EA (1:1) to afford tert-butyl 4-(6-{8-fluoro-2-methylimidazo[1,2-a]pyridin-6-yl}-4-oxothieno[3,2-d]pyrimidin-3-yl)-3,6-dihydro-2H-pyridine-1-carboxylate (40 mg, 7%) as a solid. LCMS (ES, m/z): 482 [M+H]⁺

### Synthesis of Compound 276

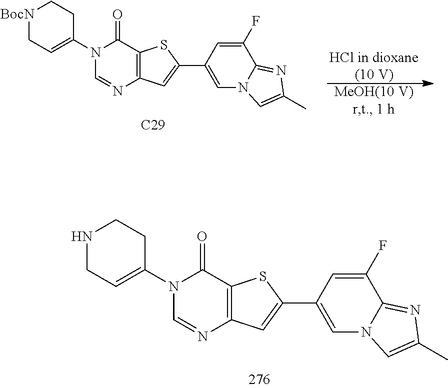 [SMILES: Cc1cn2cc(-c3cc4ncn(C5=CCN(C(=O)OC(C)(C)C)CC5)c(=O)c4s3)cc(F)c2n1 ; "C29" ; Cc1cn2cc(-c3cc4ncn(C5=CCNCC5)c(=O)c4s3)cc(F)c2n1]

A solution of tert-butyl 4-(6-{8-fluoro-2-methylimidazo[1,2-a]pyridin-6-yl}-4-oxothieno[3,2-d]pyrimidin-3-yl)-3,6-dihydro-2H-pyridine-1-carboxylate (40 mg, 0.083 mmol, 1.00 equiv) in MeOH (1 mL), HCl(gas) in 1,4-dioxane (1 mL) was stirred for 1 h at r.t. The resulting mixture was concentrated under reduced pressure. The crude product was purified by Prep-HPLC with the following conditions (Column: XBridge Prep OBD C18 Column, 30×150 mm, 5 m; Mobile Phase A: water (10 mmol/L NH₄HCO₃), Mobile Phase B: ACN; Flow rate: 60 mL/min; Gradient: 5% B to 45% B in 8 min, 45% B; Wave Length: 220 nm; RT1(min): 5.72) to afford 6-{8-fluoro-2-methylimidazo[1,2-a]pyridin-6-yl}-3-(1,2,3,6-tetrahydropyridin-4-yl)thieno[3,2-d]pyrimidin-4-one (6.9 mg, 22%) as a solid.LCMS (ES, m/z): 382 [M+H]⁺¹H NMR (400 MHz, Chloroform-d) δ 8.26 (d, J=1.5 Hz, 1H), 8.02 (s, 1H), 7.52 (m, 1H), 7.43 (m, 1H), 7.16 (dd, J=10.8, 1.4 Hz, 1H), 5.97 (s, 1H), 3.62 (d, J=2.9 Hz, 2H), 3.19 (t, J=5.6 Hz, 2H), 2.53-2.48 (m, 5H).

### Example 56: Synthesis of Compound 281

### Synthesis of Intermediate C30

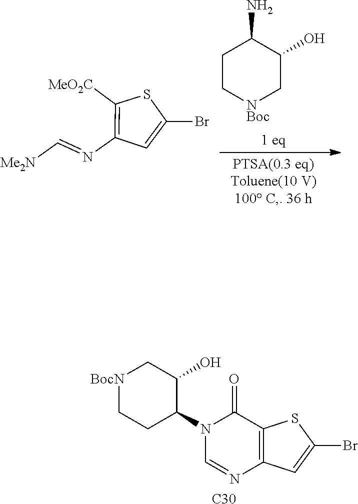

A solution of methyl 5-bromo-3-[(E)-[(dimethylamino)methylidene]amino]thiophene-2-carboxylate (1 g, 3.435 mmol, 1 equiv), tert-butyl (3S,4S)-4-amino-3-hydroxypiperidine-1-carboxylate (0.74 g, 3.421 mmol, 1.00 equiv) and PTSA (0.18 g, 1.045 mmol, 0.30 equiv) in Toluene (10 mL) was stirred for 36 h at 100° C. The mixture was allowed to cool down to room temperature. The resulting mixture was concentrated under reduced pressure. The residue was purified by silica gel column chromatography, eluted with PE/EA (1:1) to afford tert-butyl (3S,4S)-4-{6-bromo-4-oxothieno[3,2-d]pyrimidin-3-yl}-3-hydroxypiperidine-1-carboxylate (560 mg, 38%) as a solid. LCMS (ES, m/z): 430 [M+H]⁺

### Synthesis of Intermediate C31

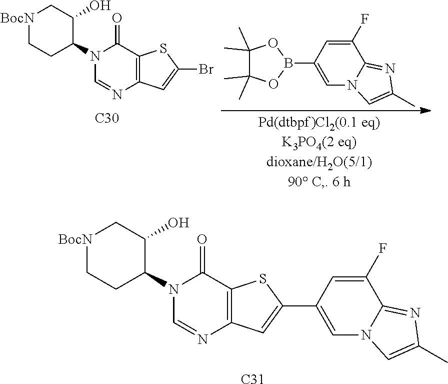

A solution of tert-butyl (3S,4S)-4-{6-bromo-4-oxothieno[3,2-d]pyrimidin-3-yl}-3-hydroxypiperidine-1-carboxylate (250 mg, 0.581 mmol, 1 equiv), 8-fluoro-2-methyl-6-(4,4,5,5-tetramethyl-1,3,2-dioxaborolan-2-yl)imidazo[1,2-a]pyridine (321 mg, 1.162 mmol, 2 equiv), Pd(DtBPF)Cl₂ (38 mg, 0.058 mmol, 0.1 equiv) and K₃PO₄ (247 mg, 1.162 mmol, 2 equiv) in dioxane (5 mL) and H₂O (1 mL) was stirred for 6 h at 90° C. under nitrogen atmosphere. The mixture was allowed to cool down to room temperature. The resulting mixture was poured into water (30 mL). The precipitated solids were collected by filtration and washed with water (3×10 mL). The resulting mixture was concentrated under reduced pressure. The crude product was re-crystallized from MeOH (10 mL) to afford tert-butyl (3S,4S)-4-(6-{8-fluoro-2-methylimidazo[1,2-a]pyridin-6-yl}-4-oxothieno[3,2-d]pyrimidin-3-yl)-3-hydroxypiperidine-1-carboxylate (200 mg, 69%) as a solid. LCMS (ES, m/z): 500 [M+H]⁺

### Synthesis of Compound 281

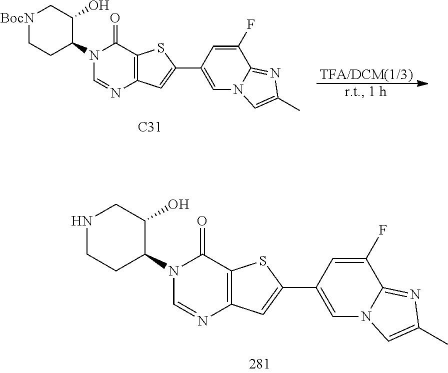 [SMILES: Cc1cn2cc(-c3cc4ncn([C@H]5CCN(C(=O)OC(C)(C)C)C[C@@H]5O)c(=O)c4s3)cc(F)c2n1 ; "C31" ; Cc1cn2cc(-c3cc4ncn([C@H]5CCNC[C@@H]5O)c(=O)c4s3)cc(F)c2n1]

A solution of tert-butyl (3S,4S)-4-(6-{8-fluoro-2-methylimidazo[1,2-a]pyridin-6-yl}-4-oxothieno [3,2-d]pyrimidin-3-yl)-3-hydroxypiperidine-1-carboxylate (200 mg, 0.400 mmol, 1 equiv) in DCM (3 mL), CF₃COOH (1 mL) was stirred for 1 h at room temperature. The resulting mixture was concentrated under reduced pressure. The residue was purified by reverse flash chromatography with the following conditions: column, C18 silica gel; mobile phase, ACN in water, 10% to 50% gradient in 40 min; detector, UV 254 nm. This resulted in 6-{8-fluoro-2-methylimidazo[1,2-a]pyridin-6-yl}-3-[(3S,4S)-3-hydroxypiperidin-4-yl]thieno[3,2-d]pyrimidin-4-one (9.7 mg, 6%) as a solid. LCMS (ES, m/z): 400 [M+H]^{+ 1}H NMR (400 MHz, DMSO-d₆) δ 9.02 (d, J=1.6 Hz, 1H), 8.46 (s, 1H), 7.89 (s, 1H), 7.86 (d, 1H), 7.72 (dd, J=12.1, 1.6 Hz, 1H), 5.07 (d, J=5.4 Hz, 1H), 4.44 (s, 1H), 3.95 (s, 1H), 3.29 (s, 1H), 3.13 (d, J=4.8 Hz, 1H), 2.97 (d, J=12.3 Hz, 1H), 2.47 (d, J=4.5 Hz, 1H), 2.38 (d, J=0.9 Hz, 3H), 2.35 (s, 1H), 1.96 (s, 1H).

### Example 57: Synthesis of Compound 281

### Synthesis of Intermediate C32

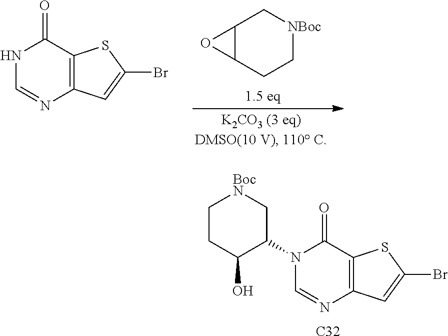

A solution of 6-bromo-3H-thieno[3,2-d]pyrimidin-4-one (1.8 g, 7.790 mmol, 1 equiv) and tert-butyl 7-oxa-3-azabicyclo[4.1.0]heptane-3-carboxylate (2.33 g, 11.685 mmol, 1.5 equiv), K₂CO₃ (3.23 g, 23.370 mmol, 3 equiv) in DMSO (18 mL) was stirred for 16 h at 110° C. The mixture was allowed to cool down to room temperature. The resulting mixture was extracted with EA (1×60 mL). The combined organic layers were washed with brine (3×60 mL), dried over anhydrous Na₂SO₄. After filtration, the filtrate was concentrated under reduced pressure. The residue was purified by silica gel column chromatography, eluted with PE/EA (5:1) to afford tert-butyl (trans)-3-{6-bromo-4-oxothieno[3,2-d]pyrimidin-3-yl}-4-hydroxypiperidine-1-carboxylate (576 mg, 17%) as a solid. LCMS (ES, m/z): 430 [M+H]⁺

### Synthesis of Intermediate C33

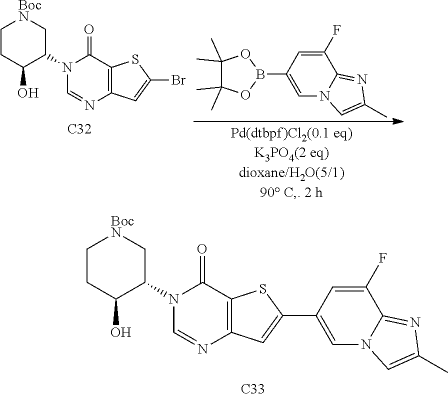

A solution of tert-butyl (trans)-3-{6-bromo-4-oxothieno[3,2-d]pyrimidin-3-yl}-4-hydroxypiperidine-1-carboxylate (200 mg, 0.465 mmol, 1 equiv) and 8-fluoro-2-methyl-6-(4,4,5,5-tetramethyl-1,3,2-dioxaborolan-2-yl)imidazo[1,2-a]pyridine (320 mg, 1.163 mmol, 2.5 equiv), Pd(DtBPF)Cl₂ (30 mg, 0.047 mmol, 0.1 equiv), K₃PO₄ (197 mg, 0.930 mmol, 2 equiv) in 1,4-dioxane (2 mL), water (0.4 mL) was stirred for 2 h at 90° C. under nitrogen atmosphere. The mixture was allowed to cool down to room temperature. The resulting mixture was extracted with EA (1×6 mL). dried over anhydrous Na₂SO₄. After filtration, the filtrate was concentrated under reduced pressure. The residue was purified by silica gel column chromatography, eluted with PE/EA (5:1) to afford tert-butyl (trans)-3-(6-{8-fluoro-2-methylimidazo [1,2-a]pyridin-6-yl}-4-oxothieno[3,2-d]pyrimidin-3-yl)-4-hydroxypiperidine-1-carboxylate (90 mg) as a solid.

LCMS (ES, m/z): 500 [M+H]⁺

### Synthesis of Compound 281

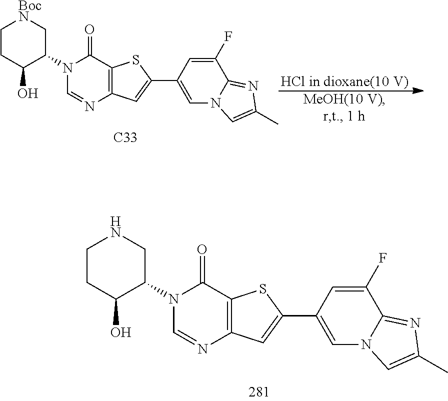 [SMILES: Cc1cn2cc(-c3cc4ncn([C@H]5CN(C(=O)OC(C)(C)C)CC[C@@H]5O)c(=O)c4s3)cc(F)c2n1 ; "C33" ; Cc1cn2cc(-c3cc4ncn([C@H]5CNCC[C@@H]5O)c(=O)c4s3)cc(F)c2n1]

A solution of tert-butyl (trans)-3-(6-{8-fluoro-2-methylimidazo[1,2-a]pyridin-6-yl}-4-oxothieno[3,2-d]pyrimidin-3-yl)-4-hydroxypiperidine-1-carboxylate (90 mg, 0.180 mmol, 1 equiv) in MeOH (1 mL), HCl(gas) in 1,4-dioxane (1 mL) was stirred for 1 h at room temperature. The resulting mixture was concentrated under reduced pressure. The crude product was purified by Prep-HPLC with the following conditions (Column: YMC-Actus Triart C18, 30×150 mm, 5 m; Mobile Phase A: water (10 mmol/L NH₄HCO₃), Mobile Phase B: ACN; Flow rate: 60 mL/min; Gradient: 3% B to 3% B in 2 min, 3% B to 45% B in 8 min, 45% B; Wave Length: 220 nm; RT1(min): 7.98) to afford 6-{8-fluoro-2-methylimidazo[1,2-a]pyridin-6-yl}-3-[(trans)-4-hydroxypiperidin-3-yl]thieno[3,2-d]pyrimidin-4-one (6.1 mg, 8%) as a solid. LCMS (ES, m/z): 400 [M+H]^{+ 1}H NMR (400 MHz, DMSO-d₆) δ 9.02 (d, J=1.6 Hz, 1H), 8.50 (s, 1H), 7.91-7.84 (m, 2H), 7.72 (dd, J=12.1, 1.6 Hz, 1H), 5.05 (d, J=5.5 Hz, 1H), 4.44 (s, 1H), 4.05 (s, 1H), 3.01-2.88 (m, 2H), 2.81 (s, 1H), 2.68 (s, 1H), 2.38 (d, J=0.9 Hz, 3H), 1.93 (s, 1H), 1.50-1.36 (m, 1H).

### Example 58: Synthesis of Compound 277

### Synthesis of Intermediate C34

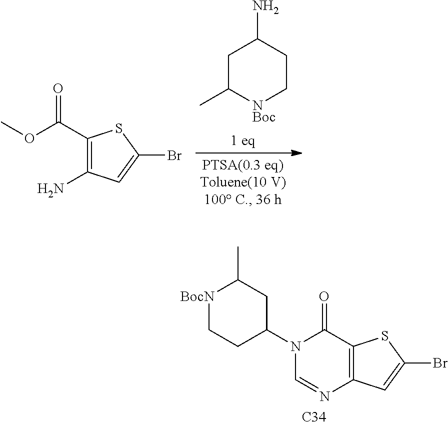 [SMILES: COC(=O)c1sc(Br)cc1N ; CC1CC(N)CCN1C(=O)OC(C)(C)C ; CC1CC(n2cnc3cc(Br)sc3c2=O)CCN1C(=O)OC(C)(C)C ; "C34"]

A solution of methyl 3-amino-5-bromothiophene-2-carboxylate (1 g, 4.236 mmol, 1.00 equiv) and tert-butyl 4-amino-2-methylpiperidine-1-carboxylate (0.91 g, 4.236 mmol, 1 equiv), PTSA (0.22 g, 1.271 mmol, 0.3 equiv) in Toluene (10 mL) was stirred for 36 h at 100 degrees C. The mixture was allowed to cool down to r.t. The resulting mixture was concentrated under reduced pressure. The residue was purified by silica gel column chromatography, eluted with PE/EA (1:1) to afford tert-butyl 4-{6-bromo-4-oxothieno[3,2-d]pyrimidin-3-yl}-2-methylpiperidine-1-carboxylate (660 mg, 36.38%) as a yellow solid. This resulted in tert-butyl 4-{6-bromo-4-oxothieno[3,2-d]pyrimidin-3-yl}-2-methylpiperidine-1-carboxylate (660 mg, 36%) as a solid. LCMS (ES, m/z): 428 [M+H]⁺

### Synthesis of Intermediate C35A/B

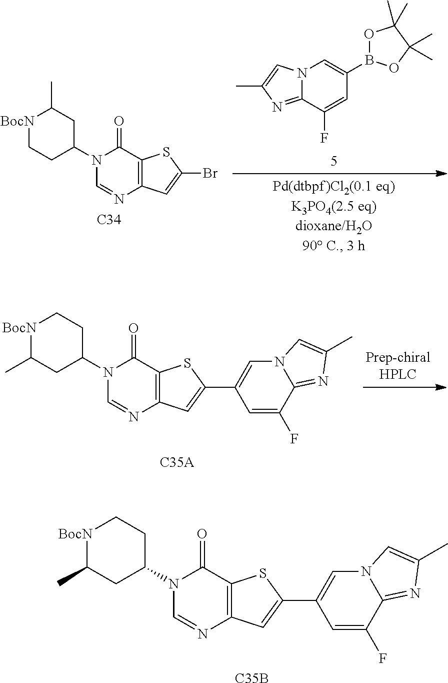

A solution of tert-butyl 4-{6-bromo-4-oxothieno[3,2-d]pyrimidin-3-yl}-2-methylpiperidine-1-carboxylate (660 mg, 1.541 mmol, 1.00 equiv) and 8-fluoro-2-methyl-6-(4,4,5,5-tetramethyl-1,3,2-dioxaborolan-2-yl)imidazo[1,2-a]pyridine (425 mg, 1.541 mmol, 1 equiv), Pd(DtBPF)Cl₂ (100 mg, 1.233 mmol, 0.1 equiv), K₃PO₄ (817.65 mg, 3.853 mmol, 2.5 equiv) in 1,4-dioxane (7 mL) and H₂O (1.2 mL) was stirred for 3 h at 90 degrees C. under nitrogen atmosphere. The mixture was allowed to cool down to r.t. The resulting mixture was extracted with EA (1×20 mL). dried over anhydrous Na₂SO₄. After filtration, the filtrate was concentrated under reduced pressure. The crude product was purified by Prep-HPLC with the following conditions (Column: CHIRAL ART Cellulose-SB, 2×25 cm, 5 m; Mobile Phase A: MtBE (0.1% DEA)-HPLC, Mobile Phase B: MeOH-HPLC; Flow rate: 20 mL/min; Gradient: 10% B to 10% B in 18.5 min; Wave Length: 220/254 nm; RT1(min): 13.8; RT2(min): 16.4; Sample Solvent: DCM-HPLC; Injection Volume: 0.22 mL; Number Of Runs: 26) to afford tert-butyl (trans)-4-(6-{8-fluoro-2-methylimidazo[1,2-a]pyridin-6-yl}-4-oxothieno[3,2-d]pyrimidin-3-yl)-2-methylpiperidine-1-carboxylate as a solid. LCMS (ES, m/z): 498 [M+H]⁺

### Synthesis of Compound 277

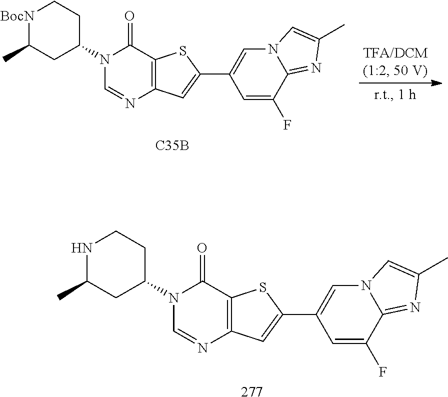

A solution of tert-butyl (trans)-4-(6-{8-fluoro-2-methylimidazo[1,2-a]pyridin-6-yl}-4-oxothieno[3,2-d]pyrimidin-3-yl)-2-methylpiperidine-1-carboxylate (12 mg, 0.024 mmol, 1.00 equiv) in DCM (1 mL), CF₃COOH (0.5 mL) was stirred for 1 h at r.t. The resulting mixture was concentrated under reduced pressure. The crude product was purified by Prep-HPLC with the following conditions (Column: YMC-Actus Triart C18, 30×150 mm, 5 m; Mobile Phase A: water (10 mmol/L NH₄HCO₃), Mobile Phase B: ACN; Flow rate: 60 mL/min; Gradient: 5% B to 45% B in 8 min, 45% B; Wave Length: 220 nm; RT1(min): 6.32) to afford 6-{8-fluoro-2-methylimidazo[1,2-a]pyridin-6-yl}-3-[(trans)-2-methylpiperidin-4-yl]thieno[3,2-d]pyrimidin-4-one (5.8 mg, 60%) as a solid. LCMS (ES, m/z): 398 [M+H]^{+ 1}H NMR (400 MHz, DMSO-d₆) δ 9.02 (d, J=1.5 Hz, 1H), 8.51 (s, 1H), 7.91-7.83 (m, 2H), 7.72 (dd, J=12.1, 1.6 Hz, 1H), 5.01 (ddd, J=11.9, 8.0, 4.0 Hz, 1H), 3.37 (s, 1H), 2.93 (td, J=12.2, 11.0, 3.0 Hz, 1H), 2.88-2.81 (m, 1H), 2.38 (d, J=0.9 Hz, 3H), 2.08 (td, J=12.1, 5.0 Hz, 1H), 1.92 (qd, J=11.5, 4.5 Hz, 1H), 1.80 (d, J=11.8 Hz, 1H), 1.69 (s, 1H), 1.19 (d, J=6.9 Hz, 3H).

### Example 59: Synthesis of Compound 278

### Synthesis of Intermediate C36

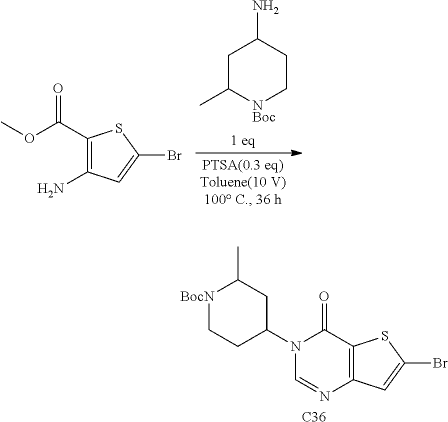 [SMILES: COC(=O)c1sc(Br)cc1N ; CC1CC(N)CCN1C(=O)OC(C)(C)C ; CC1CC(n2cnc3cc(Br)sc3c2=O)CCN1C(=O)OC(C)(C)C ; "C36"]

A solution of methyl 3-amino-5-bromothiophene-2-carboxylate (1 g, 4.236 mmol, 1.00 equiv) and tert-butyl 4-amino-2-methylpiperidine-1-carboxylate (0.91 g, 4.236 mmol, 1 equiv), PTSA (0.22 g, 1.271 mmol, 0.3 equiv) in Toluene (10 mL) was stirred for 36 h at 100 degrees C. The mixture was allowed to cool down to r.t. The resulting mixture was concentrated under reduced pressure. The residue was purified by silica gel column chromatography, eluted with PE/EA (1:1) to afford tert-butyl 4-{6-bromo-4-oxothieno[3,2-d]pyrimidin-3-yl}-2-methylpiperidine-1-carboxylate (660 mg, 36%) as a solid. LCMS (ES, m/z): 428 [M+H]⁺

### Synthesis of Intermediate C37A B

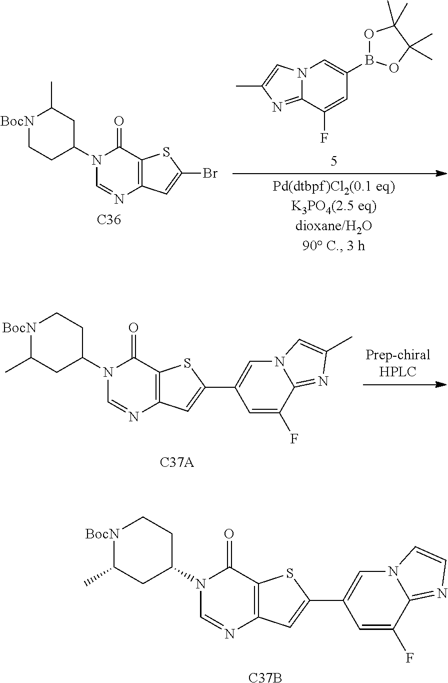

A solution of tert-butyl 4-{6-bromo-4-oxothieno[3,2-d]pyrimidin-3-yl}-2-methylpiperidine-1-carboxylate (660 mg, 1.541 mmol, 1.00 equiv) and 8-fluoro-2-methyl-6-(4,4,5,5-tetramethyl-1,3,2-dioxaborolan-2-yl)imidazo[1,2-a]pyridine (425 mg, 1.541 mmol, 1 equiv), Pd(DtBPF)Cl₂ (100 mg, 1.233 mmol, 0.80 equiv), K₃PO₄ (817.65 mg, 3.853 mmol, 2.5 equiv) in 1,4-dioxane (7 mL) was stirred for 2 h at 90 degrees C. under nitrogen atmosphere. The mixture was allowed to cool down to r.t. The resulting mixture was extracted with EA (1×20 mL), dried over anhydrous Na₂SO₄. After filtration, the filtrate was concentrated under reduced pressure. The crude product was purified by Prep-HPLC with the following conditions (Column: CHIRAL ART Cellulose-SB, 2×25 cm, 5 m; Mobile Phase A: MtBE (0.1% DEA)-HPLC, Mobile Phase B: MeOH—-HPLC; Flow rate: 20 mL/min; Gradient: 10% B to 10% B in 18.5 min; Wave Length: 220/254 nm; RT1(min): 13.8; RT2(min): 16.4; Sample Solvent: DCM-HPLC; Injection Volume: 0.22 mL; Number Of Runs: 26) to afford tert-butyl (cis)-4-(6-{8-fluoro-2-methylimidazo[1,2-a]pyridin-6-yl}-4-oxothieno[3,2-d]pyrimidin-3-yl)-2-methylpiperidine-1-carboxylate as a solid. LCMS (ES, m/z): 498 [M+H]⁺

### Synthesis of Compound 278

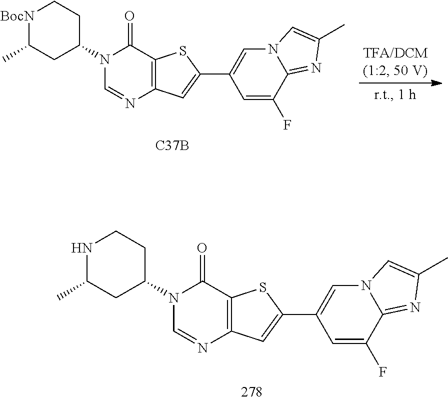

A solution of tert-butyl (cis)-4-(6-{8-fluoro-2-methylimidazo[1,2-a]pyridin-6-yl}-4-oxothieno[3,2-d]pyrimidin-3-yl)-2-methylpiperidine-1-carboxylate (60 mg, 0.121 mmol, 1.00 equiv) in DCM (1 mL), CF₃COOH (0.5 mL) was stirred for 1 h at r.t. The resulting mixture was concentrated under reduced pressure. The residue was purified by trituration with MeOH (5 mL). The residue was purified by trituration with MTBE (5 mL). This resulted in 6-{8-fluoro-2-methylimidazo[1,2-a]pyridin-6-yl}-3-[(cis)-2-methylpiperidin-4-yl]thieno[3,2-d]pyrimidin-4-one hydrochloride (31.1 mg, 59%) as a solid. LCMS (ES, m/z): 398 [M+H]^{+ 1}H NMR (400 MHz, Deuterium Oxide) δ 8.86 (s, 1H), 8.39 (s, 1H), 8.01-7.94 (m, 1H), 7.90 (d, J=2.4 Hz, 1H), 7.69-7.61 (m, 1H), 5.02-4.90 (m, 1H), 3.67-3.57 (m, 1H), 3.57-3.44 (m, 1H), 3.23 (td, J=12.9, 4.2 Hz, 1H), 2.51-2.46 (m, 3H), 2.38-2.23 (m, 3H), 2.22-2.08 (m, 1H), 1.37 (d, J=6.5 Hz, 3H).

### Example 60: Synthesis of Compound 269

### Synthesis of Intermediate C38

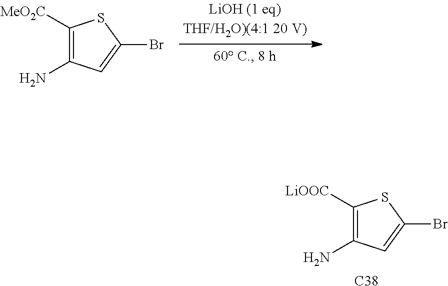

Into a 25-mL round-bottom flask purged and maintained with an inert atmosphere of nitrogen, was placed methyl 2-amino-4-bromo-6-fluorobenzoate (500 mg, 2.016 mmol, 1.00 equiv), LiOH (96.54 mg, 4.032 mmol, 2 equiv), THE (8 mL) and H₂O (2 mL). The resulting solution was stirred for 8 hr at 60° C. The resulting mixture was concentrated in vacuum. and used directly without further purification. This resulted in 600 mg (crude) of lithio 3-amino-5-bromothiophene-2-carboxylate as a solid. LCMS (ES, m/z): 222 [M+H]⁺

### Synthesis of Intermediate C39

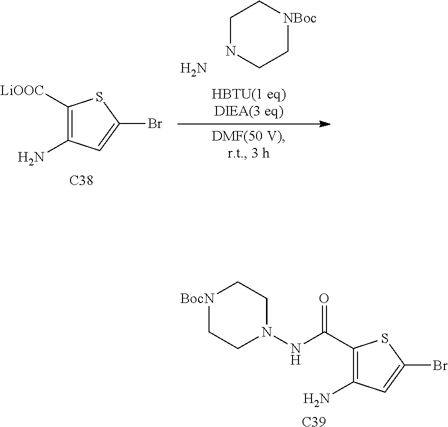

Into a 25-mL round-bottom flask purged and maintained with an inert atmosphere of nitrogen, was placed lithio 3-amino-5-bromothiophene-2-carboxylate (300 mg, 1.316 mmol, 1.00 equiv), HBTU (748.54 mg, 1.974 mmol, 1.5 equiv) in DMF (5 mL) was added DIEA (510.19 mg, 3.948 mmol, 3 equiv). The resulting solution was stirred for 3 h at room temperature. The reaction was then quenched by the addition of 20 mL of water, The resulting solution was extracted with 3×20 mL of ethyl acetate and the organic layers combined and dried over anhydrous sodium sulfate and concentrated. The residue was applied onto a silica gel column with ethyl acetate/hexane (1:1). This resulted in 350 mg (66%) of tert-butyl 4-(3-amino-5-bromothiophene-2-amido)piperazine-1-carboxylate as a solid. LCMS (ES, m/z): 405 [M+H]⁺

### Synthesis of Intermediate C40

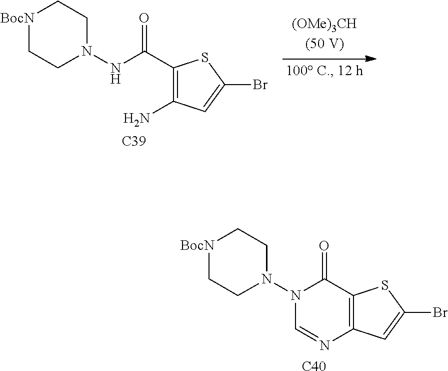 [SMILES: CC(C)(C)OC(=O)N1CCN(NC(=O)c2sc(Br)cc2N)CC1 ; "C39" ; CC(C)(C)OC(=O)N1CCN(n2cnc3cc(Br)sc3c2=O)CC1 ; "C40"]

Into a 25-mL round-bottom flask purged and maintained with an inert atmosphere of nitrogen, was placed tert-butyl 4-(3-amino-5-bromothiophene-2-amido)piperazine-1-carboxylate (350 mg, 0.864 mmol, 1.00 equiv) in trimethyl orthoformate (5 mL). The resulting solution was concentrated in vcuum. The residue was applied onto a silica gel column with ethyl acetate/hexane (1:2). This resulted in 300 mg (84%) of tert-butyl 4-{6-bromo-4-oxothieno[3,2-d]pyrimidin-3-yl}piperazine-1-carboxylate as a solid. LCMS (ES, m/z): 417 [M+H]⁺

### Synthesis of Intermediate C41

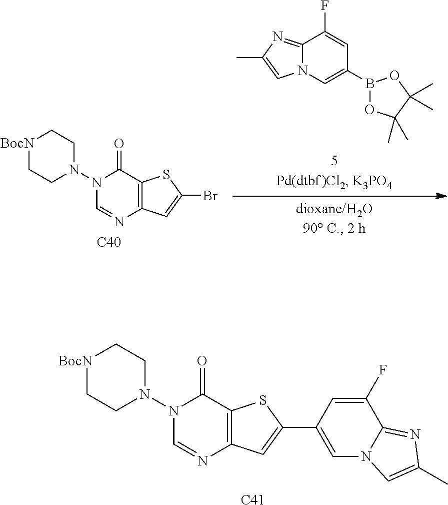

Into a 10-mL round-bottom flask purged and maintained with an inert atmosphere of nitrogen, was placed tert-butyl 4-{6-bromo-4-oxothieno[3,2-d]pyrimidin-3-yl}piperazine-1-carboxylate (100 mg, 0.241 mmol, 1.00 equiv), 8-fluoro-2-methyl-6-(4,4,5,5-tetramethyl-1,3,2-dioxaborolan-2-yl)imidazo[1,2-a]pyridine (99.73 mg, 0.361 mmol, 1.5 equiv), K₃PO₄ (153 mg, 0.723 mmol, 3 equiv) and Pd(DtBPF)Cl₂ (15.69 mg, 0.024 mmol, 0.1 equiv) in1,4-dioxane (5 mL) H₂O (1 mL). The resulting solution was stirred for 2 hr at 90° C. The resulting mixture was concentrated. The residue was applied onto a silica gel column with ethyl acetate/hexane (1:1). This resulted in 60 mg (51.4%) of tert-butyl 4-(6-{8-fluoro-2-methylimidazo[1,2-a]pyridin-6-yl}-4-oxothieno[3,2-d]pyrimidin-3-yl)piperazine-1-carboxylate as a solid. LCMS (ES, m/z): 485 [M+H]⁺

### Synthesis of Compound 269

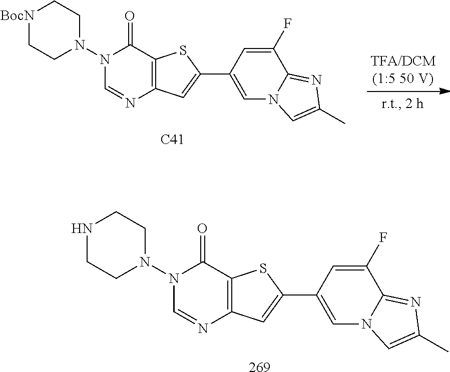 [SMILES: Cc1cn2cc(-c3cc4ncn(N5CCN(C(=O)OC(C)(C)C)CC5)c(=O)c4s3)cc(F)c2n1 ; "C41" ; Cc1cn2cc(-c3cc4ncn(N5CCNCC5)c(=O)c4s3)cc(F)c2n1 ; "269"]

Into a 25-mL round-bottom flask purged and maintained with an inert atmosphere of nitrogen, was placed tert-butyl 4-(6-{8-fluoro-2-methylimidazo[1,2-a]pyridin-6-yl}-4-oxothieno[3,2-d]pyrimidin-3-yl)piperazine-1-carboxylate (40 mg, 0.083 mmol, 1.00 equiv),TFA (0.5 mL), DCM (2.5 mL). The resulting solution was stirred for 1 h at 25 degrees C. The resulting mixture was concentrated. The crude product was purified by Chiral-Prep-HPLC with the following conditions (2#SHIMADZU (HPLC-01)): Column, Xselect CSH OBD Column 30*150 mm Sum, n; mobile phase, Water(0.05% HCl) and ACN (hold 3% ACN in 2 min, up to 20% in 6 min); Gradient:3 B to 33 B in 6 min; 220 nm; RT1:4.75. This resulted in 15 mg (47%) of 6-{8-fluoro-2-methylimidazo[1,2-a]pyridin-6-yl}-3-(piperazin-1-yl)thieno[3,2-d]pyrimidin-4-one as a solid. LCMS (ES, m/z): 385 [M+H]^{+ 1}H NMR (400 MHz, DMSO-d₆) δ 9.38 (s, 1H), 9.24 (s, 1H), 8.95 (s, 1H), 8.32 (s, 1H), 8.10 (d, J=11.7 Hz, 1H), 8.02 (d, J=5.7 Hz, 2H), 3.39-3.18 (m, 8H), 2.46 (s, 3H). ¹⁹F NMR (376 MHz, DMSO) δ 131.22.

### Example 61: Synthesis of Compound 270

### Synthesis of Intermediate C42

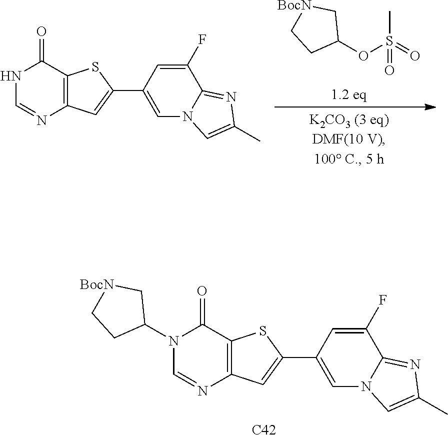 [SMILES: Cc1cn2cc(-c3cc4nc[nH]c(=O)c4s3)cc(F)c2n1 ; CC(C)(C)OC(=O)N1CCC(OS(C)(=O)=O)C1 ; Cc1cn2cc(-c3cc4ncn(C5CCN(C(=O)OC(C)(C)C)C5)c(=O)c4s3)cc(F)c2n1 ; "C42"]

A solution of 6-{8-fluoro-2-methylimidazo[1,2-a]pyridin-6-yl}-3H-thieno[3,2-d]pyrimidin-4-one (150 mg, 0.499 mmol, 1.00 equiv) and tert-butyl 3-(methanesulfonyloxy)pyrrolidine-1-carboxylate (159 mg, 0.599 mmol, 1.2 equiv), K₂CO₃ (207 mg, 1.497 mmol, 3 equiv) in DMF (1.5 mL) was stirred for 5 h at 100 degrees C. The mixture was allowed to cool down to r.t. The resulting mixture was extracted with EA (1×5 mL). dried over anhydrous Na₂SO₄. After filtration, the filtrate was concentrated under reduced pressure. The residue was purified by silica gel column chromatography, eluted with PE/EA (5:1) to afford tert-butyl 3-(6-{8-fluoro-2-methylimidazo[1,2-a]pyridin-6-yl}-4-oxothieno[3,2-d]pyrimidin-3-yl)pyrrolidine-1-carboxylate (17 mg, 7%) as a solid. LCMS (ES, m/z):470 [M+H]⁺

### Synthesis of Intermediate C43

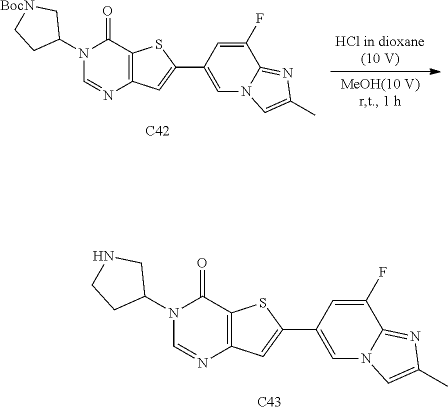 [SMILES: Cc1cn2cc(-c3cc4ncn(C5CCN(C(=O)OC(C)(C)C)C5)c(=O)c4s3)cc(F)c2n1 ; "C42" ; Cc1cn2cc(-c3cc4ncn(C5CCNC5)c(=O)c4s3)cc(F)c2n1 ; "C43"]

A solution of tert-butyl 3-(6-{8-fluoro-2-methylimidazo[1,2-a]pyridin-6-yl}-4-oxothieno[3,2-d]pyrimidin-3-yl)pyrrolidine-1-carboxylate (17 mg, 0.036 mmol, 1.00 equiv) in MeOH (1 mL) was stirred for 1 h at r.t. The resulting mixture was concentrated under reduced pressure. The crude product was purified by Prep-HPLC with the following conditions (Column: YMC-Actus Triart C18, 30×150 mm, 5 m; Mobile Phase A: water (10 mmol/L NH₄HCO₃), Mobile Phase B: ACN; Flow rate: 60 mL/min; Gradient: 5% B to 70% B in 8 min, 70% B; Wave Length: 220 nm; RT1(min): 4.52) to afford 6-{8-fluoro-2-methylimidazo[1,2-a]pyridin-6-yl}-3-(pyrrolidin-3-yl)thieno[3,2-d]pyrimidin-4-one (4.5 mg, 33.34%) as a solid. LCMS (ES, m/z):370 [M+H]^{+ 1}H NMR (400 MHz, DMSO-d₆) δ 9.02 (d, J=1.6 Hz, 1H), 8.53 (s, 1H), 7.92-7.84 (m, 2H), 7.72 (dd, J=12.2, 1.6 Hz, 1H), 5.20 (td, J=8.2, 4.4 Hz, 1H), 3.88-3.47 (s, 1H) 3.19-3.09 (m, 2H), 3.00 (dd, J=11.7, 3.9 Hz, 1H), 2.85 (ddd, J=10.7, 8.3, 6.7 Hz, 1H), 2.39 (d, J=0.8 Hz, 3H), 2.32-2.20 (m, 1H), 1.91 (dt, J=12.7, 6.9 Hz, 1H)ₒ

### Example 62: Synthesis of Compound 275

### Synthesis of Intermediate C43

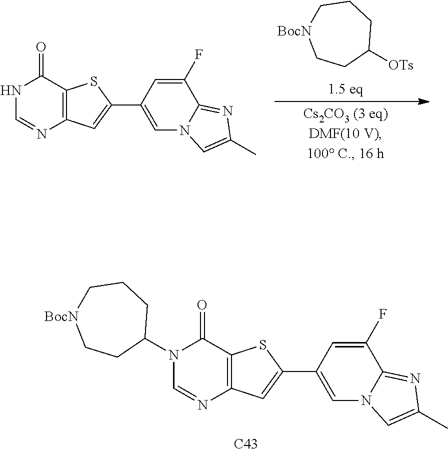 [SMILES: Cc1cn2cc(-c3cc4nc[nH]c(=O)c4s3)cc(F)c2n1 ; Cc1ccc(S(=O)(=O)OC2CCCN(C(=O)OC(C)(C)C)CC2)cc1 ; Cc1cn2cc(-c3cc4ncn(C5CCCN(C(=O)OC(C)(C)C)CC5)c(=O)c4s3)cc(F)c2n1 ; "C43"]

A solution of 6-{8-fluoro-2-methylimidazo[1,2-a]pyridin-6-yl}-3H-thieno[3,2-d]pyrimidin-4-one (150 mg, 0.499 mmol, 1.00 equiv) and tert-butyl 4-[(4-methylbenzenesulfonyl)oxy]azepane-1-carboxylate (277 mg, 0.748 mmol, 1.5 equiv) in DMF (1.5 mL) was stirred for 16 h at 100 degrees C. The resulting mixture was extracted with EA (1×5 mL). The combined organic layers were washed with brine (3×5 mL), dried over anhydrous Na₂SO₄. After filtration, the filtrate was concentrated under reduced pressure. The residue was purified by silica gel column chromatography, eluted with PE/EA (1:1) to afford tert-butyl 4-(6-{8-fluoro-2-methylimidazo[1,2-a]pyridin-6-yl}-4-oxothieno[3,2-d]pyrimidin-3-yl)azepane-1-carboxylate (20 mg, 8%) as a solid. LCMS (ES, m/z):498 [M+H]⁺

### Synthesis of Compound 275

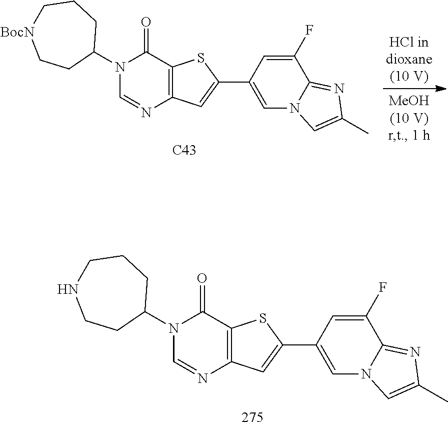 [SMILES: Cc1cn2cc(-c3cc4ncn(C5CCCN(C(=O)OC(C)(C)C)CC5)c(=O)c4s3)cc(F)c2n1 ; "C43" ; Cc1cn2cc(-c3cc4ncn(C5CCCNCC5)c(=O)c4s3)cc(F)c2n1 ; "275"]

A solution of tert-butyl 4-(6-{8-fluoro-2-methylimidazo[1,2-a]pyridin-6-yl}-4-oxothieno[3,2-d]pyrimidin-3-yl)azepane-1-carboxylate (20 mg, 0.040 mmol, 1.00 equiv) in MeOH (1 mL), HCl(gas) in 1,4-dioxane (1 mL) was stirred for 1 h at r.t. The resulting mixture was concentrated under reduced pressure. The crude product was purified by Prep-HPLC with the following conditions (Column: XBridge Prep OBD C18 Column, 30×150 mm, 5 m; Mobile Phase A: water (10 mmol/L NH₄HCO₃), Mobile Phase B: ACN; Flow rate: 60 mL/min; Gradient: 3% B to 43% B in 8 min, 43% B; Wave Length: 220 nm; RT1(min): 6.42) to afford 3-(azepan-4-yl)-6-{8-fluoro-2-methylimidazo[1,2-a]pyridin-6-yl}thieno[3,2-d]pyrimidin-4-one (3.4 mg, 21.07%) as a solid. LCMS (ES, m/z):398 [M+H]⁺¹H NMR (400 MHz, DMSO-d₆) δ 9.02 (d, J=1.6 Hz, 1H), 8.56 (s, 1H), 7.99 (s, 1H), 7.87 (d, 1H), 7.73 (dd, J=12.2, 1.6 Hz, 1H), 4.91 (s, 1H), 4.73 (s, 1H), 2.89 (dt, J=11.1, 6.2 Hz, 2H), 2.79 (dd, J=13.4, 4.9 Hz, 2H), 2.39 (s, 3H), 2.24 (d, J=11.4 Hz, 1H), 2.02 (s, 2H), 2.07-1.96 (m, 2H), 1.87 (m, 1H).

### Example 63: Synthesis of Compound 279

### Synthesis of Intermediate C44

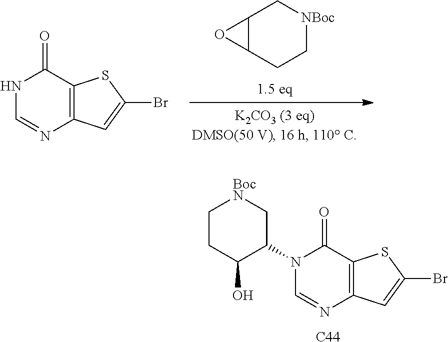

A solution of 6-bromo-3H-thieno[3,2-d]pyrimidin-4-one (1 g, 4.328 mmol, 1 equiv) and tert-butyl 7-oxa-3-azabicyclo[4.1.0]heptane-3-carboxylate (1.29 g, 6.492 mmol, 1.5 equiv), K₂CO₃ (1.79 g, 12.984 mmol, 3 equiv) in DMSO (50 mL) was stirred for 16 h at 110° C. The mixture was allowed to cool down to room temperature. The resulting mixture was extracted with EA (1×60 mL). The combined organic layers were washed with brine (3×60 mL), dried over anhydrous Na₂SO₄. After filtration, the filtrate was concentrated under reduced pressure. The residue was purified by silica gel column chromatography, eluted with PE/EA (5:1) to afford tert-butyl (4S)-3-{6-bromo-4-oxothieno[3,2-d]pyrimidin-3-yl}-4-hydroxypiperidine-1-carboxylate (683 mg, 37%) as a solid. LCMS (ES, m/z): 430 [M+H]⁺

### Synthesis of Intermediate C45

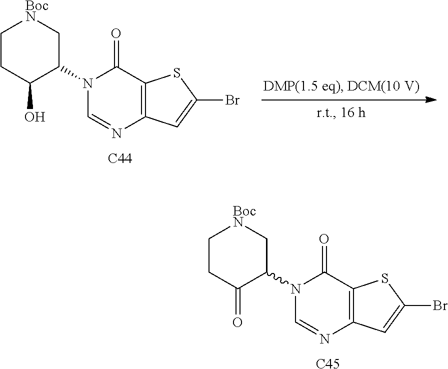

A solution of tert-butyl (4S)-3-{6-bromo-4-oxothieno[3,2-d]pyrimidin-3-yl}-4-hydroxypiperidine-1-carboxylate (683 mg, 1.587 mmol, 1 equiv) and DMP (1 g, 2.381 mmol, 1.5 equiv) in DCM (7 mL) was stirred for 16 h at room temperature under nitrogen atmosphere. The reaction was quenched by the addition of water (30 mL) at room temperature. The resulting mixture was extracted with EA (1×30 mL), dried over anhydrous Na₂SO₄. After filtration, the filtrate was concentrated under reduced pressure. The residue was purified by silica gel column chromatography, eluted with PE/EA (1:1) to afford tert-butyl 3-{6-bromo-4-oxothieno[3,2-d]pyrimidin-3-yl}-4-oxopiperidine-1-carboxylate (120 mg, 18%) as a solid. LCMS (ES, m/z): 428 [M+H]⁺

### Synthesis of Intermediate C46

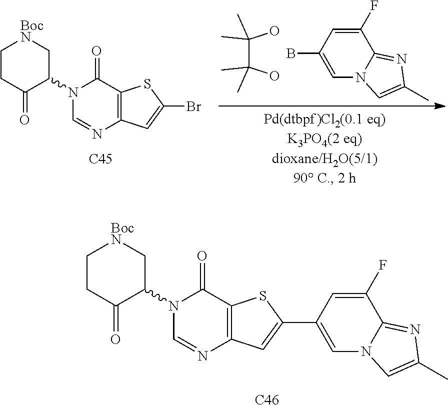

A solution of tert-butyl 3-{6-bromo-4-oxothieno[3,2-d]pyrimidin-3-yl}-4-oxopiperidine-1-carboxylate (120 mg, 0.280 mmol, 1 equiv) and 8-fluoro-2-methyl-6-(4,4,5,5-tetramethyl-1,3,2-dioxaborolan-2-yl) imidazo[1,2-a]pyridine (193 mg, 0.700 mmol, 2.5 equiv), Pd(dtbpf)Cl₂ (18 mg, 0.028 mmol, 0.1 equiv), K₃PO₄ (119 mg, 0.560 mmol, 2 equiv) in 1,4-dioxane (1 mL) and water (0.2 mL) was stirred for 2 h at 90° C. under nitrogen atmosphere. The mixture was allowed to cool down to room temperature. The resulting mixture was extracted with EA (1×5 mL), dried over anhydrous Na₂SO₄. After filtration, the filtrate was concentrated under reduced pressure. The residue was purified by silica gel column chromatography, eluted with PE/EA (5:1) to afford tert-butyl 3-(6-{8-fluoro-2-methylimidazo[1,2-a]pyridin-6-yl}-4-oxothieno[3,2-d]pyrimidin-3-yl)-4-oxopiperidine-1-carboxylate (60 mg, 43%) as a solid. LCMS (ES, m/z): 498 [M+H]⁺

### Synthesis of Compound 279

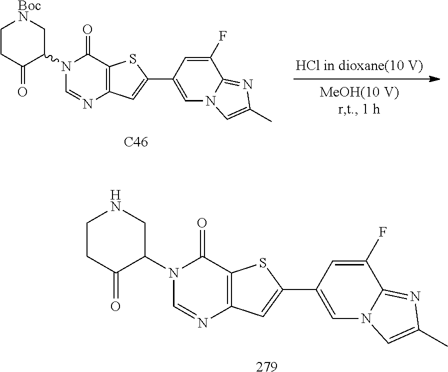 [SMILES: Cc1cn2cc(-c3cc4ncn(C5CN(C(=O)OC(C)(C)C)CCC5=O)c(=O)c4s3)cc(F)c2n1 ; "C46" ; Cc1cn2cc(-c3cc4ncn(C5CNCCC5=O)c(=O)c4s3)cc(F)c2n1 ; "279"]

A solution tert-butyl 3-(6-{8-fluoro-2-methylimidazo[1,2-a]pyridin-6-yl}-4-oxothieno[3,2-d]pyrimidin-3-yl)-4-oxopiperidine-1-carboxylate (60 mg, 0.121 mmol, 1.00 equiv) in MeOH (0.6 mL), HCl(gas) in 1,4-dioxane (0.6 mL) was stirred for 1 h at r.t. The resulting mixture was concentrated under reduced pressure. The crude product was purified by Prep-HPLC with the following conditions (Column: XBridge Prep OBD C18 Column, 30×150 mm, 5 m; Mobile Phase A: water (10 mmol/L NH₄HCO₃), Mobile Phase B: ACN; Flow rate: 60 mL/min; Gradient: 5% B to 40% B in 8 min, 40% B; Wave Length: 220 nm; RT1(min): 6.42) to afford 3-(6-{8-fluoro-2-methylimidazo[1,2-a]pyridin-6-yl}-4-oxothieno [3,2-d]pyrimidin-3-yl)piperidin-4-one (19.9 mg, 40%) as a solid.LCMS (ES, m/z): 398 [M+H]^{+ 1} H NMR (400 MHz, DMSO-d₆) δ 9.02 (d, J=1.6 Hz, 1H), 8.36 (s, 1H), 7.92 (s, 1H), 7.87 (d, 1H), 7.72 (dd, J 12.1, 1.6 Hz, 1H), 5.47 (dd, J=10.8, 6.9 Hz, 1H), 3.44 (dd, J=12.1, 7.0 Hz, 1H), 3.28 (s, 1H), 2.95 (t, J=12.5 Hz, 2H), 2.66-2.59 (m, 1H), 2.46 (s, 1H), 2.38 (d, J=0.8 Hz, 3H).

### Example 64: Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation of a new junction with an alternative exon (AJ). Real-time qPCR assays were used to detect these splicing switches and interrogate the potency of various compounds towards different target genes. A high-throughput real time quantitative PCR (RT-qPCR) assay was developed to measure these two isoforms of the mRNA (CJ and AJ) for exemplary genes, such as HTT, SMN2, and MYB, together with a control housekeeping gene, GAPDH or GUSB or PPIA, used for normalization. Briefly, the A673 or K562 cell line was treated with various compounds described herein (e.g., compounds of Formula (I)). After treatment, the levels of the HTT, MYB, or SMN2 mRNA targets were determined from each sample of cell lysate by cDNA synthesis followed by qPCR.

### Materials:

Cells-to-C_{T} 1-step kit: ThermoFisher A25602, Cells-to-C_{T} lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905_ml)—used for K562/suspension cell lines
GUSB: VIC-PL, ThermoFisher 4326320E (Assay: Hs99999908_ml)—used for K562/suspension cell lines
PPIA: VIC-PL, ThermoFisher 4326316E (Assay: Hs99999904_ml)—used for A673/adherent cell lines

### Probe/Primer Sequences

Canonical junction (CJ)
HTT Primer 1:
TCCTCCTGAGAAAGAGAAGGAC
HTT Primer 2:
GCCTGGAGATCCAGACTCA
HTT CY5-Probe:
/5Cy5/TGGCAACCCTTGAGGCCCTGTCCT/3IAbRQSp/
MYB Primer 1:
CCTCATTGGTCACAAATTGACTG
MYB Primer 2:
TGGAGAGCTTTCTAAGATTGACC
MYB CY5-Probe:
/5Cy5/AGGAAAATACTGTTTTTAGAACCCCAG/3IAbRQSp/
Alternative junction (AJ)
HTT Primer 1:
TCCTGAGAAAGAGAAGGACATTG
HTT Primer 2:
CTGTGGGCTCCTGTAGAAATC
HTT FAM-Probe:
/56-FAM/TGGCAACCC/ZEN/TTGAGAGGCAAGCCCT/3IABkFQ/
MYB Primer 1:
CAACACCATTTCATAGAGACCAGAC
MYB Primer 2:
GTTCTAAAATCATCCCTTGGCTTCTAAT
MYB FAM-Probe:
/56-FAM/AAATACTGT/ZEN/ATAGGACCTCTTCTGACATCC/
3IABkFQ/

### Description

The A673 cell line was cultured in DMEM with 10% FBS. Cells were diluted with full growth media and plated in a 96-well plate (15,000 cells in 100 ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate. Compound was transferred from the intermediate plate to the cell plate with the top dose at a final concentration of 10 uM in the well. Final DMSO concentration was kept at or below 0.25%. Final volume was 100 uL for 96-well plate and 50 uL for 384-well plate. The cell plate was then placed in an incubator at 37° C. with 5% CO2 for 24 hours.

The cells were then gently washed with 50 uL-100 uL cold PBS before proceeding to addition of lysis buffer. 30 uL-50 μL of room temperature lysis buffer with DNAse I (and optionally RNAsin) was added to each well. Cells were shaken/mixed thoroughly at room temperature for 5-10 minutes for lysis to take place and then 3 uL-5 μL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. An appropriate volume of 3×lysis buffer (10 mM Tris, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 μL. The table below summarizes the components of the RT-qPCR reactions:

| Component | 1X |
|---|---|
| Taqman 1-step RT-qPCR mix (4X) | 2.5 |
| 20X AJ Primers + Probe (FAM) | 0.5 |
| 20X CJ Primers + Probe (CY5) | 0.5 |
| 20X PPIA Control (VIC) | 0.5 |
| Cell lysate (1X) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time |
|---|---|---|---|
| RT step | 1 | 50° C. | 5 min |
| RT inactivation/initial | 1 | 95° C. | 20 sec |
| denaturation | | | |
| Amplification | 40 | 95° C. | 3 sec |
| | | 60° C. | 30 sec |

The data analysis was performed by first determining the ΔCt vs the housekeeper gene. This ΔCt was then normalized against the DMSO control (ΔΔCt) and converted to RQ (relative quantification) using the 2\hat{ }(−ΔΔCt) equation. The RQ were then converted to a percentage response by arbitrarily setting an assay window of 3.5 and 4.0 ΔCt for HTT-CJ and MYB-CJ respectively and an assay window of 9 and 3 ΔCt for HTT-AJ and MYB-AJ in 96 well format (50,000 K562 cells/well and 15,000 A673 cells per well) and an assay window of 3 and 4 ΔCt for HTT-CJ and MYB-CJ respectively and an assay window of 5 and 3 ΔCt for HTT-AJ and MYB-AJ respectively in 384 well format (8,000 K562 cells/well example). These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 500 response in AJ increase) while the decrease in CJ mRNA levels is reported as IC₅₀ (compound concentration having 50% response in CJ decrease).

A summary of these results is illustrated in Table 3, wherein “A” represents an AC₅₀/IC₅₀ of less than 100 nM; “B” represents an AC₅₀/IC₅₀ of between 100 nM and 1 μM; and “C” represents an AC₅₀/IC₅₀ of between 1 μM and 10 μM; and “D” represents an AC₅₀/IC₅₀ of greater than 10 μM.

TABLE 4
Modulation of RNA Splicing by Exemplary Compounds

| Compound | HTT AJ | HTT CJ | MYB AJ | MYB CJ |
|---|---|---|---|---|
| No. | AC₅₀ (nM) | IC₅₀ (nM) | AC₅₀ (nM) | IC₅₀ (nM) |
| 109 | D | D | C | D |
| 157 | D | D | D | D |
| 159 | C | C | C | C |
| 182 | C | C | — | — |
| 183 | D | D | D | D |
| 184 | D | D | C | D |
| 205 | C | C | C | C |
| 206 | C | C | C | C |
| 207 | D | D | D | C |
| 208 | C | C | C | C |
| 209 | C | C | C | C |
| 210 | C | C | C | C |
| 211 | C | C | B | C |
| 212 | C | C | C | C |
| 213 | D | D | D | D |
| 214 | D | C | C | C |
| 215 | C | C | C | C |
| 216 | C | C | C | C |
| 217 | C | C | C | C |
| 218 | C | C | C | C |
| 225 | D | D | D | D |
| 226 | B | B | B | B |
| 227 | D | D | D | D |
| 228 | C | C | C | C |
| 229 | D | D | D | D |
| 230 | D | D | B | B |
| 231 | B | B | B | B |
| 232 | A | A | B | B |
| 233 | A | A | A | B |
| 234 | B | B | A | B |
| 235 | A | A | B | B |
| 236 | B | A | B | B |
| 237 | B | B | A | B |
| 238 | B | B | B | B |
| 239 | C | C | B | C |
| 250 | B | B | B | B |
| 251 | C | C | D | C |
| 253 | C | C | C | C |
| 256 | D | D | D | D |
| 257 | C | C | C | C |
| 258 | B | B | B | B |
| 259 | C | C | B | B |
| 260 | C | C | C | C |
| 261 | C | B | B | B |
| 262 | B | B | B | B |
| 263 | C | C | C | C |
| 264 | C | C | C | C |
| 265 | B | B | B | B |
| 267 | B | B | B | B |
| 268 | D | D | C | D |
| 269 | B | B | B | B |
| 270 | B | B | B | B |
| 271 | C | C | C | B |
| 272 | D | C | C | C |
| 273 | C | B | B | B |
| 274 | C | D | D | D |
| 275 | B | B | B | B |
| 276 | B | B | B | B |
| 277 | B | B | B | B |
| 278 | B | B | B | B |
| 279 | D | D | D | D |
| 280 | B | B | B | B |
| 281 | C | C | C | C |
| 800 | D | D | D | D |
| 801 | D | D | D | D |
| 802 | B | B | B | B |
| 803 | D | D | D | D |
| 804 | B | B | B | B |
| 805 | B | B | B | B |
| 806 | B | B | B | B |
| 807 | B | B | B | B |
| 808 | C | B | B | B |
| 809 | C | C | C | C |
| 810 | B | B | B | B |
| 811 | C | C | C | C |
| 812 | D | D | D | D |

Additional studies were carried out for a larger panel of genes using the protocol provided above. The junction between flanking upstream and downstream exons was used to design canonical junction qPCR assays. At least one of the forward primer, reverse primer or the CY5-labeled 5′ nuclease probe (with 3′ quencher such as ZEN/Iowa Black FQ) was designed to overlap with the exon junction to capture the CJ mRNA transcript. BLAST was used to confirm the specificity of the probeset and parameters such as melting temperature, GC content, amplicon size, and primer dimer formation are considered during their design. Data for the decrease in CJ mRNA levels for four exemplary genes (HTT, SMN2, MYB, and Target C) analyzed in this panel are reported as IC₅₀ (compound concentration having 50% response in CJ decrease).

A summary of the results from the panel is illustrated in Table 4, wherein “A” represents an IC₅₀ of less than 100 nM; “B” represents an IC₅₀ of between 100 nM and 1 μM; and “C” represents an IC₅₀ of between 1 μM and 10 μM; and “D” represents an IC₅₀ of greater than 10 μM.

TABLE 5
Modulation of RNA Splicing by Exemplary Compounds

| Compound | | | | Target |
|---|---|---|---|---|
| No. | HTT | MYB | SMN2 | C |
| 109 | D | D | D | D |
| 157 | D | D | D | D |
| 159 | C | C | C | C |
| 182 | C | — | C | — |
| 183 | D | D | C | D |
| 184 | D | D | C | — |
| 205 | C | C | C | C |
| 206 | C | C | B | C |
| 207 | D | C | B | D |
| 208 | C | C | C | C |
| 209 | C | C | C | C |
| 210 | C | C | C | C |
| 211 | C | C | B | C |
| 212 | C | C | C | C |
| 213 | D | D | C | D |
| 214 | C | C | B | D |
| 215 | C | C | B | C |
| 216 | C | C | C | C |
| 217 | C | C | C | C |
| 218 | C | C | C | C |
| 225 | D | D | D | D |
| 226 | B | B | B | B |
| 227 | D | D | D | D |
| 228 | C | C | C | C |
| 229 | D | D | D | D |
| 230 | D | D | D | D |
| 231 | B | B | C | C |
| 232 | A | B | A | B |
| 233 | A | B | A | B |
| 234 | B | B | A | C |
| 235 | A | B | A | B |
| 236 | A | B | A | B |
| 237 | B | B | A | C |
| 238 | B | B | A | C |
| 239 | C | C | A | D |
| 250 | B | B | B | B |
| 251 | C | C | B | D |
| 253 | C | C | B | C |
| 256 | D | D | C | D |
| 257 | C | C | C | C |
| 258 | B | B | A | C |
| 259 | C | B | A | C |
| 260 | C | C | C | C |
| 261 | B | B | A | C |
| 262 | B | B | B | B |
| 263 | C | C | C | C |
| 264 | C | C | B | C |
| 265 | B | B | A | B |
| 267 | B | B | B | B |
| 268 | D | D | B | D |
| 269 | B | B | A | C |
| 270 | B | B | B | B |
| 271 | C | B | A | C |
| 272 | C | C | A | D |
| 273 | B | B | B | C |
| 274 | D | D | C | D |
| 275 | B | B | A | B |
| 276 | B | B | A | B |
| 277 | B | B | A | C |
| 278 | B | B | A | B |
| 279 | D | D | D | D |
| 280 | B | B | B | B |
| 281 | C | C | C | C |
| 800 | D | D | D | D |
| 801 | D | D | D | D |
| 802 | B | B | A | B |
| 803 | D | D | D | D |
| 804 | B | B | B | B |
| 805 | B | B | A | C |
| 806 | B | B | B | B |
| 807 | B | B | A | C |
| 808 | B | B | A | C |
| 809 | C | C | B | C |
| 810 | B | B | B | B |
| 811 | C | C | B | D |
| 812 | D | D | D | D |

### Example 65: Evaluating Effect of Exemplary Compounds on Protein Abundance

Compounds described herein were used to screen for effects on quantitative protein abundance using a HiBit assay system (Promega). Quantitative protein abundance was determined by measuring the protein levels of HiBit-tagged protein targets expressed in cell culture via luminescence using the Nano-Glo HiBiT Lytic Detection System, which uses a split complementation assay format to reconstitute NanoBiT enzyme to generate a luminescent signal. A protein abundance assay was developed such that endogenous protein targets could be modified with the HiBiT peptide tag and their abundance could be assessed after compound treatment. Briefly, K562 cell lines containing a HiBiT-modification were treated with various compounds described herein (e.g., compounds of Formulas (I), (II), or (III)). After treatment for 24 hours, the protein abundance of a specific target was determined by measuring luminescence.

### Materials:

Promega Nano-Glo HiBiT Lytic Detection System (cat #N3030)
Corning 384-well TC-treated microplates (cat #3570)
Synthego Engineered Cells Knock-In Clones

TABLE 5
Design of genetically modified HiBiT cell lines

| | | | Guide | Guide | |
|---|---|---|---|---|---|
| Cell | | Modifi- | RNA | RNA cut | |
| Line | Gene | cation | Sequence | location | Donor Sequence |
| K562 | MYB | HiBiT | GCGCCA | chr6: | CGGTGCGGTCCCCGCGGCTC |
| | | | TGGCCC | 135,181,526 | TCGGCGGAGCCCCGCGCCCG |
| | | | GAAGAC | | CCGCGCCATGgtgagcggct |
| | | | CC | | ggcggctgttcaagaagatt |
| | | | | | agcGGCAGCTCCGGAGGATC |
| | | | | | TAGCGGCGCCCGAAGACCCC |
| | | | | | GGCACAGgtaacggggagcc |
| | | | | | gggcgggcggccgaggg |
| K562 | HTT | HiBiT | CAGCTT | chr4: | CGAGTCGGCCCGAGGCCTCC |
| | | | TTCCAG | 3,074,830 | GGGGACTGCCGTGCCGGGCG |
| | | | GGTCGC | | GGAGACCGCCATGgtgagcg |
| | | | CA | | gctggcggctgttcaagaag |
| | | | | | attagcGGCAGCTCCGGAGG |
| | | | | | ATCTAGCGGCGCGACCCTGG |
| | | | | | AAAAGCTGATGAAGGCCTTC |
| | | | | | GAGTCCCTCAAGTCCTTCCA |

### Description:

Cells were maintained in IMDM with 10% FBS. Before the assay, cells were diluted with phenolphthalein-free growth media (IMDM+1% FBS media) and were seeded in a 384-well plate at a density of 10000 cells/well (for each cell line listed in Table 5). Each compound was prepared as a 10-point 3-fold serial dilution in DMSO with the top dose at a final concentration of 10 μM in the well. Unmodified K562 cells were added at the previously specified density with DMSO to serve as an assay baseline and positive control (PC) and DMSO only with the respective modified cell lines was added to the negative control (NC) columns. Final DMSO concentration was kept at or below 0.25%. Treated cell plates were placed in an incubator at 37° C. with 5% CO₂ for 24 hours. After 24 hours, 25 μL of Complete HiBit Lytic reagent was added to each well at room temperature (e.g. one plate requiring 10 mL Lytic Buffer, 100 μL LgBiT Protein, 200 μL Lytic Substrate), shaken for 5 minutes at 600 RPM, then left to sit for 10 minutes for signal to stabilize before reading on a Spark Cyto plate reader (Tecan) with a 500 ms measurement time.

To determine compound effects on protein abundance of each target in Table 5, the percent response for each respective cell line was calculated at each compound concentration as follows:

$$\% response = 100 * ( S - PC ) / ( NC - P C )$$

For the normalized response at each concentration, a four-parameter logistical regression was fit to the data and the response was interpolated at the 50% value to determine a concentration for protein abundance at 50% (IC₅₀) the untreated control. A summary of the results for protein abundance can be prepared and labeled, with compounds following in the following representative categories: A represents <100 nM; B represents 100-1000 nM; C represents 1000-9999 nM; and D represents greater than 10 μM.

TABLE 6
Effect of Exemplary Compounds on Protein Abundance

| Com- | | | | Com- | | | |
|---|---|---|---|---|---|---|---|
| pound | | Target | | pound | | Target | |
| No. | MYB | C | HTT | No. | MYB | C | HTT |
| 109 | D | D | D | 210 | C | C | C |
| 157 | D | D | D | 211 | C | C | C |
| 159 | C | C | C | 212 | C | C | C |
| 205 | C | C | C | 213 | D | D | D |
| 206 | C | C | C | 214 | D | D | D |
| 207 | D | D | D | 215 | C | D | D |
| 208 | C | C | C | 216 | C | C | C |
| 209 | C | C | C | 217 | C | C | C |
| 218 | C | C | C | 263 | C | C | C |
| 250 | B | B | B | 264 | C | C | C |
| 256 | D | D | D | 265 | B | B | B |
| 267 | B | B | B | 269 | B | B | B |
| 268 | D | D | D | 270 | B | B | B |
| 283 | C | C | C | 271 | C | C | C |
| 225 | D | D | D | 272 | C | C | C |
| 226 | B | B | B | 273 | C | C | C |
| 227 | D | D | D | 274 | D | D | D |
| 228 | C | C | C | 275 | B | B | B |
| 229 | D | D | D | 276 | B | B | B |
| 230 | D | D | D | 277 | B | B | B |
| 231 | B | B | B | 278 | B | B | B |
| 232 | B | B | A | 279 | D | D | D |
| 233 | A | A | A | 334 | D | D | D |
| 234 | A | B | B | 335 | D | D | D |
| 235 | B | B | B | 336 | B | B | B |
| 236 | A | B | A | 800 | D | D | D |
| 237 | A | B | B | 801 | D | D | D |
| 238 | B | C | B | 802 | B | B | A |
| 239 | C | D | C | 804 | B | B | B |
| 251 | C | D | C | 805 | B | B | B |
| 253 | C | C | C | 806 | B | B | B |
| 257 | C | C | C | 807 | B | C | B |
| 258 | B | C | B | 808 | B | C | B |
| 259 | B | C | B | 809 | C | C | C |
| 260 | C | C | C | 810 | B | B | B |
| 261 | B | C | B | 811 | C | D | C |
| 262 | B | B | B | 812 | B | B | B |

### Example 66: Investigating Effect of Exemplary Compounds on Cell Viability

Compounds described herein were screened for toxicity in K562 (human chronic myelogenous leukemia) cells using a Cell Titer Glo 2.0 assay.

### Materials:

Promega CellTiter-Gbo® 2.0 Cell Viability Assay (cat #G9241)
Corning 384-well TC-treated microplates (cat #3570)

### Description:

Cells were plated at 500 cells/well (K562 cells) in 45 μL of IMDM supplemented with 10% FBS in a 384-well opaque plate. Wells containing only medium were used as a blank control. Test compounds (e.g., compounds of Formula (I), (II), and (III)) were first serially diluted in DMSO then diluted 1:100 with IMDM+10% FBS. The final concentration of DMSO was 0.1% in each well. The cells were incubated for 72 hours at 37° C. and 5% CO₂ before assaying with Cell Titer Glo 2.0 reagent.

Exemplary compounds were tested and found to fall within the following ranges: compounds labeled “A” represent <100 nM; “B” represent 100-1000 nM; “C” represent 1000-9999 nM; and “D” represent greater than 10 μM in K562 cells.

TABLE 7
Effect of Exemplary Compounds on Cell Viability

| | Viability |
|---|---|
| Compound | K562 |
| No. | GI50 |
| 109 | C |
| 157 | D |
| 159 | C |
| 205 | C |
| 206 | C |
| 207 | D |
| 208 | C |
| 209 | C |
| 210 | C |
| 211 | C |
| 212 | C |
| 213 | D |
| 214 | C |
| 215 | C |
| 216 | C |
| 217 | C |
| 218 | C |
| 250 | B |
| 256 | D |
| 267 | B |
| 268 | D |
| 283 | C |
| 225 | D |
| 226 | B |
| 227 | D |
| 228 | C |
| 229 | D |
| 230 | D |
| 231 | C |
| 232 | B |
| 233 | B |
| 234 | B |
| 235 | B |
| 236 | B |
| 237 | B |
| 238 | C |
| 239 | C |
| 251 | C |
| 253 | D |
| 257 | C |
| 258 | C |
| 259 | C |
| 260 | C |
| 261 | C |
| 262 | B |
| 263 | C |
| 264 | C |
| 265 | B |
| 269 | B |
| 270 | B |
| 271 | C |
| 272 | D |
| 273 | C |
| 274 | D |
| 275 | B |
| 276 | B |
| 277 | B |
| 278 | B |
| 279 | D |
| 334 | D |
| 335 | D |
| 336 | C |
| 800 | D |
| 801 | D |
| 802 | B |
| 804 | B |
| 805 | C |
| 806 | B |
| 807 | C |
| 808 | B |
| 809 | C |
| 810 | B |
| 811 | C |
| 812 | B |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A compound of Formula (I): 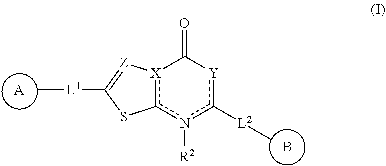
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
L¹ and L² are each independently absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴;
X is N or C;
Y is N, N(R^{5a}), C(R^{5b}), or C(R^{5b})(R^{5c}), wherein the dashed lines representing bonds in the ring comprising X and Y may be single or double bonds as valency permits;
Z is N or C(R⁶);
wherein at least one of X, Y, and Z is independently N;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷;
R² is absent, hydrogen, or C₁-C₆-alkyl;
each R³ is independently hydrogen, C₁-C₆-alkyl, or C₁-C₆-haloalkyl;
each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
R^{5a} is hydrogen, C₁-C₆-alkyl, C₁-C₆-heteroalkyl, C₁-C₆-haloalkyl;
each of R^{5b} and R^{5c} is independently hydrogen, C₁-C₆-alkyl, C₁-C₆-heteroalkyl, C₁-C₆-haloalkyl, halo, or —OR^{A};
R⁶ is hydrogen, C₁-C₆-alkyl, C₁-C₆-heteroalkyl, C₁-C₆-haloalkyl, halo, or —OR^{A};
each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁸;
each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each R^{B} and R^{C} is independently hydrogen, C₁-C₆ alkyl, C₁-C₆ heteroalkyl, cycloalkyl, heterocyclyl, —OR^{A}; or
R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁹ is independently C₁-C₆-alkyl or halo; and
x is 0, 1, or 2.

2. The compound of claim 1, wherein one of A and B is independently cycloalkyl, heterocyclyl, or heteroaryl.

3. The compound of any one of claims 1-2, wherein one of A and B is independently a nitrogen-containing heterocyclyl or nitrogen-containing heteroaryl.

4. The compound of any one of claims 1-3, wherein one of A and B is independently selected from 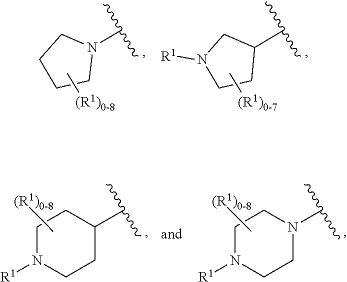
wherein R¹ is as described in claim 1.

5. The compound of any one of claims 1-4, wherein one of A and B is independently 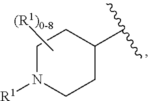
wherein R¹ is as described in claim 1.

6. The compound of any one of claims 1-5, wherein one of A and B is independently selected from

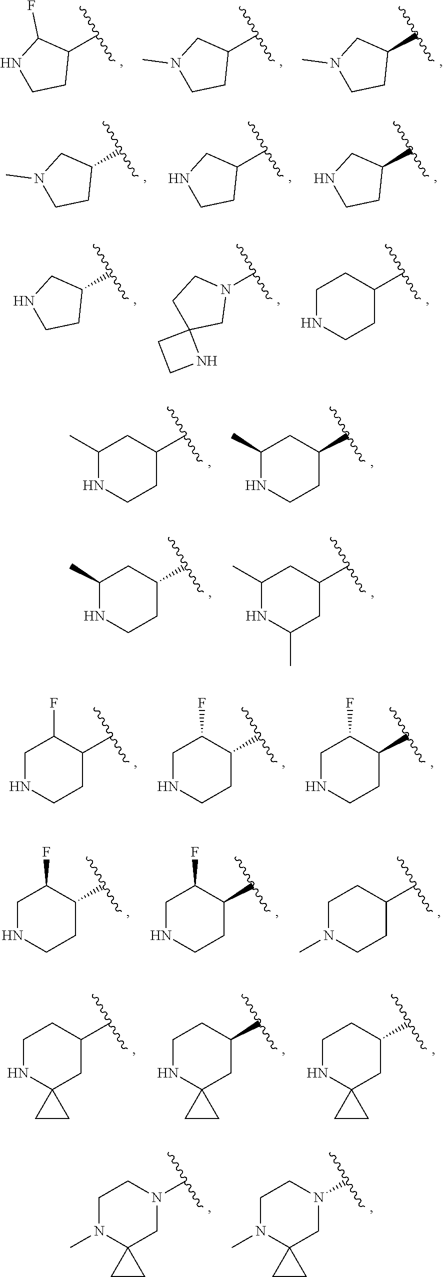

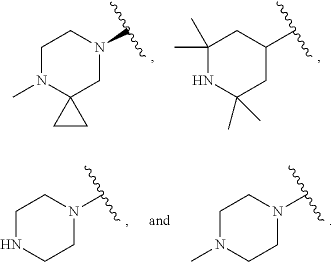

7. The compound of any one of claims 1-6, wherein one of A and B is independently selected from

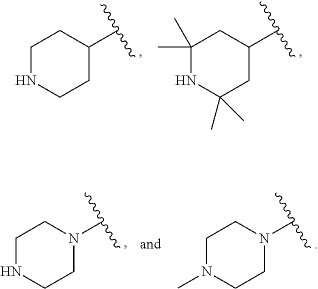

8. The compound of any one of claims 1-5, wherein one of A and B is independently selected from

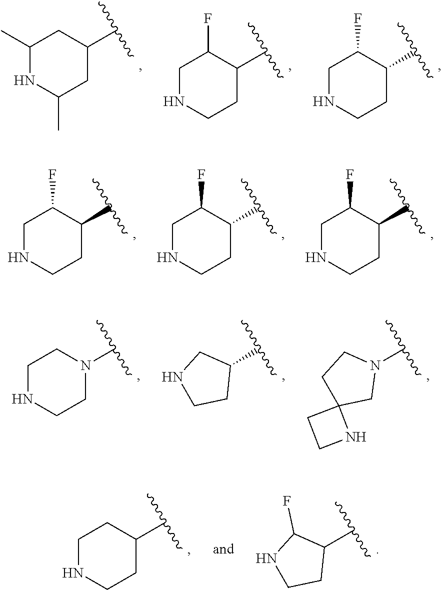

9. The compound of any one of claims 1-8, wherein A is nitrogen-containing heterocyclyl.

10. The compound of any one of claims 1-9, wherein one of A and B is independently selected from

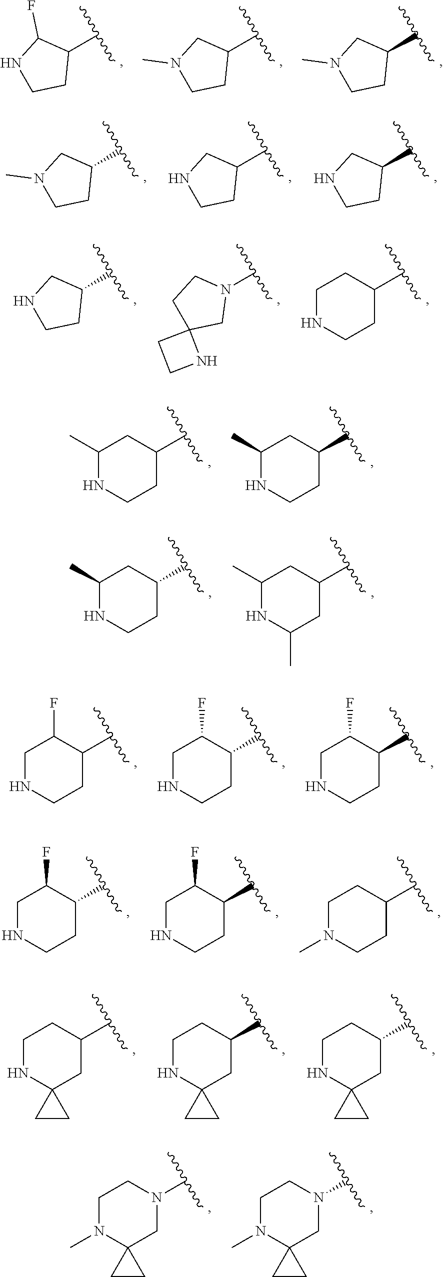

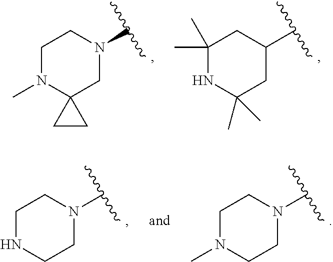

11. The compound of any one of claims 1-10, wherein one of A and B is independently selected from

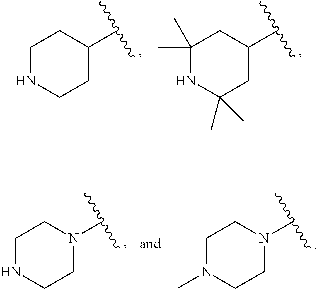

12. The compound of any one of claims 1-11, wherein one of A and B is independently selected from

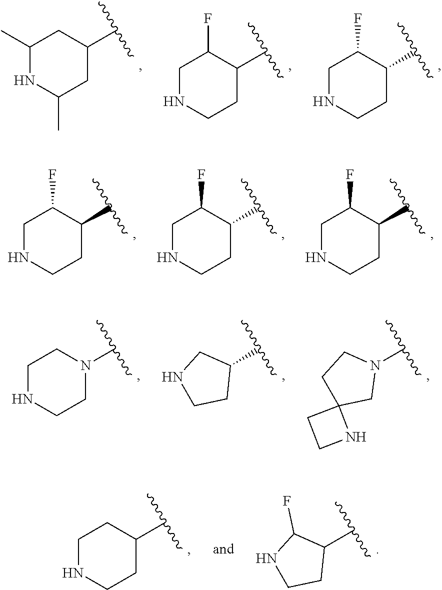

13. The compound of any one of claims 1-2, wherein one of A and B is independently cycloalkyl.

14. The compound of claim 13, wherein one of A and B is independently 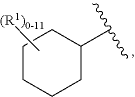
wherein R¹ is as described in claim 1.

15. The compound of any one of claims 13-14, wherein one of A and B is independently selected from 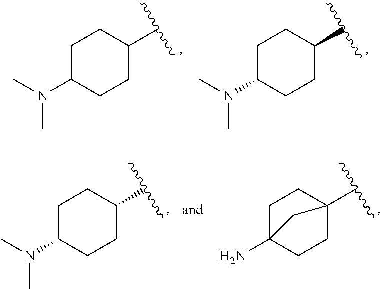
wherein R¹ is as described in claim 1.

16. The compound of any one of claims 1-15, wherein one of A and B is independently selected from 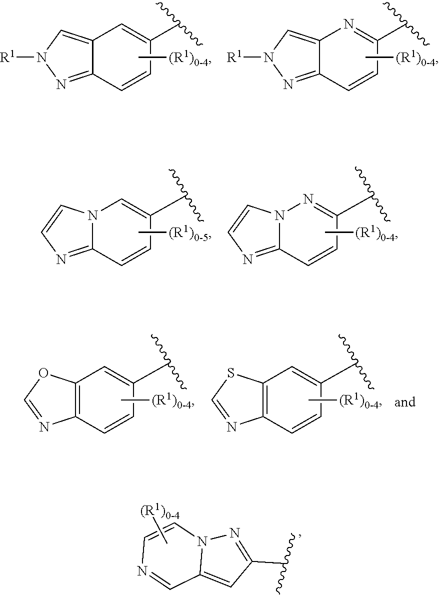
and wherein R¹ is as described in claim 1.

17. The compound of any one of claims 1-16, wherein one of A and B is independently selected from 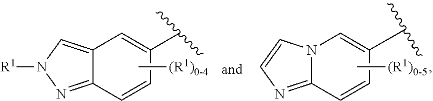
and wherein R¹ is as described in claim 1.

18. The compound of any one of claims 1-16, wherein one of A and B is independently selected from 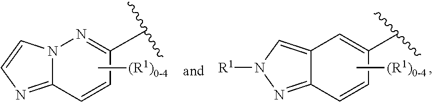
wherein R¹ is as described in claim 1.

19. The compound of any one of claims 1-18, wherein one of A and B is independently selected from

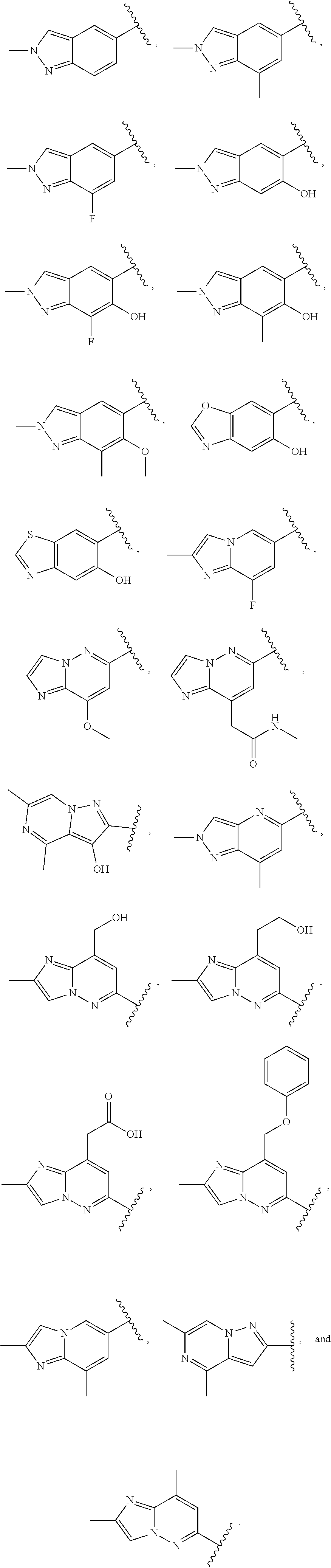

20. The compound of any one of claims 1-19, wherein one of A and B is independently selected from

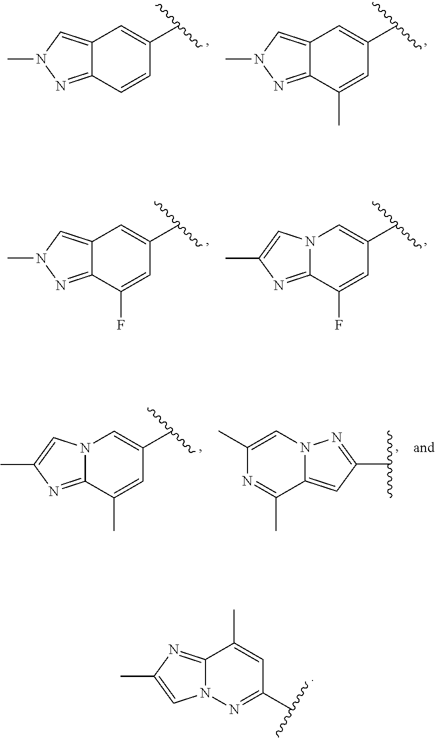

21. The compound of any one of claims 1-20 wherein one of A and B is independently selected from

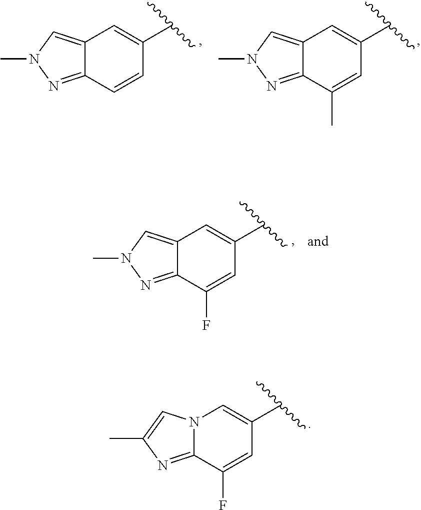 [SMILES: *c1ccc2nn(C)cc2c1 ; *c1cc(C)c2nn(C)cc2c1 ; *c1cc(F)c2nn(C)cc2c1 ; *c1cc(F)c2nc(C)cn2c1]

22. The compound of any one of claims 1-21, wherein B is a nitrogen-containing heteroaryl or nitrogen-containing heterocyclyl.

23. The compound of any one of claims 1-22, wherein B selected from selected from 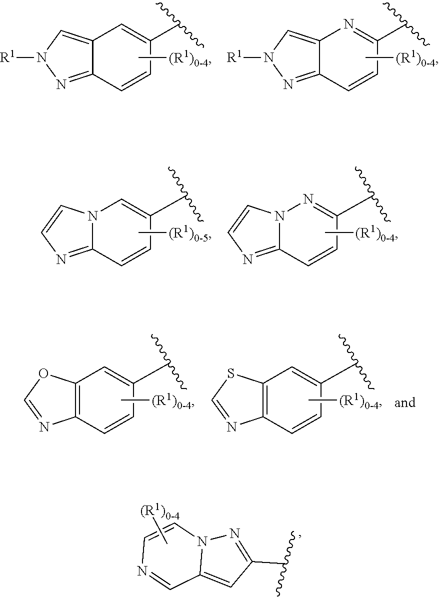
wherein R¹ is as described in claim 1.

24. The compound of any one of claims 1-23, wherein B is selected from selected from

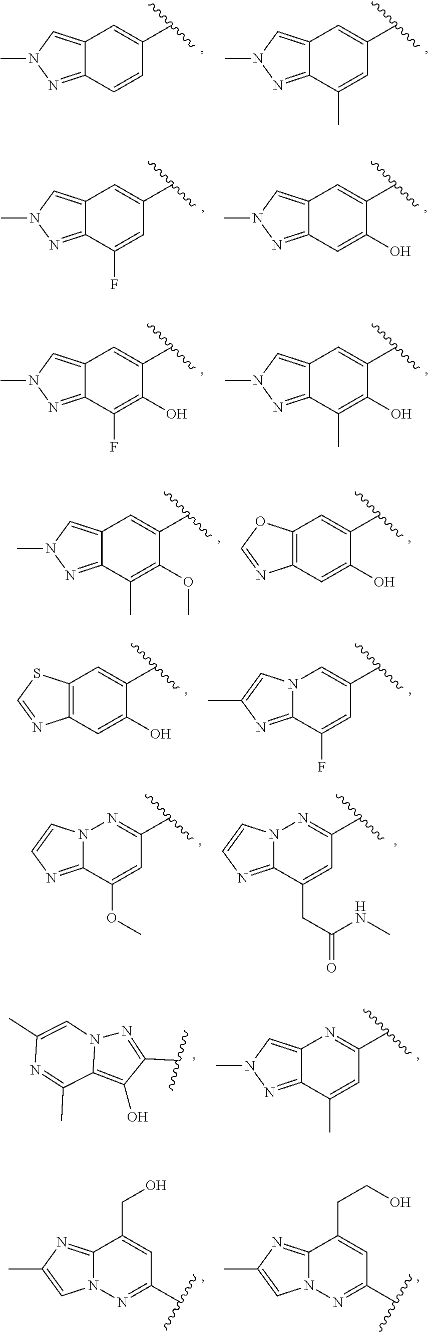

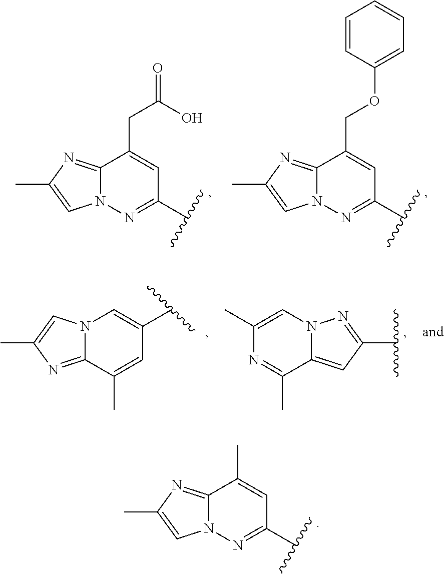 [SMILES: *c1cc(CC(=O)O)c2nc(C)cn2n1 ; *c1cc(COc2ccccc2)c2nc(C)cn2n1 ; *c1cc(C)c2nc(C)cn2c1 ; *c1cc2c(C)nc(C)cn2n1 ; *c1cc(C)c2nc(C)cn2n1]

25. The compound of any one of claims 1-24, wherein B is selected from

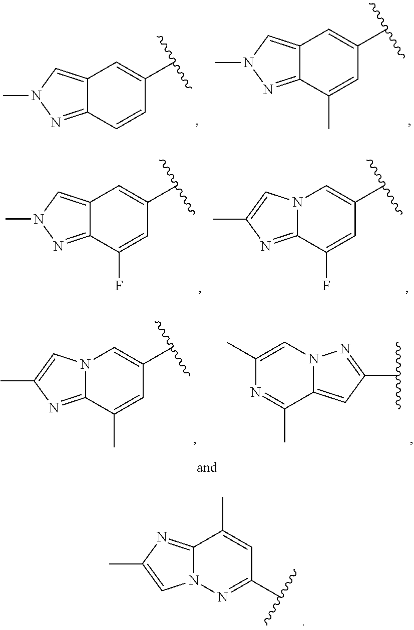

26. The compound of any one of claims 1-25, wherein one of L¹ and L² is independently absent or C₁-C₆-heteroalkylene (e.g., —N(CH₃)—).

27. The compound of any one of claims 1-26, wherein each of L¹ and L² is independently absent or C₁-C₆-heteroalkylene (e.g., —N(CH₃)—).

28. The compound of any one of claims 1-26, wherein L¹ is independently absent or C₁-C₆ heteroalkylene (e.g., —N(CH₃)—), and L² is absent.

29. The compound of any one of claims 1-28, wherein each of L¹ and L² is absent.

30. The compound of any one of claims 1-29, wherein X is N.

31. The compound of any one of claims 1-29, wherein X is C.

32. The compound of any one of claims 1-31, wherein Y is N(R^{5a}) or C(R^{5b}).

33. The compound of any one of claims 1-32, wherein Y is N(R^{5a}).

34. The compound of any one of claims 1-32, wherein Y is C(R^{5b}).

35. The compound of any one of claims 1-34, wherein Z is N.

36. The compound of any one of claims 1-34, wherein Z is C(R^{5b}).

37. The compound of any one of claims 1-26, wherein X is C, Y is N(R^{5a}), and Z is N.

38. The compound of any one of claims 1-26, wherein X is N, Y is C(R^{5b}), and Z is N.

39. The compound of any one of claims 1-26, wherein X is N, Y is C(R^{5b}), and Z is C(R⁶).

40. The compound of any one of claims 1-39, wherein R² is absent.

41. The compound of any one of the preceding claims, wherein the compound of Formula (I) is a compound of Formula (I-a): 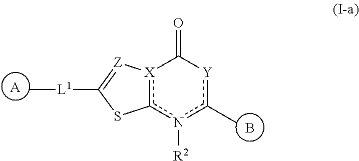
or a pharmaceutically acceptable salt, solvate, hydrate, tautomer, or stereoisomer thereof, wherein A, B, L¹, X, Y, Z, R², and subvariables thereof are as defined in claim 1.

42. The compound of any one of the preceding claims, wherein the compound of Formula (I) is a compound of Formula (I-b): 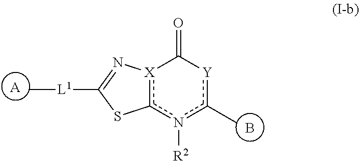
or a pharmaceutically acceptable salt, solvate, hydrate, tautomer, or stereoisomer thereof, wherein A, B, L¹, X, Y, R², and subvariables thereof are as defined in claim 1.

43. The compound of any one of the preceding claims, wherein the compound of Formula (I) is a compound of Formula (I-c): 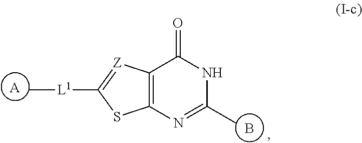
or a pharmaceutically acceptable salt, solvate, hydrate, tautomer, or stereoisomer thereof, wherein A, B, L¹, Z, and subvariables thereof are as defined in claim 1.

44. The compound of any one of the preceding claims, wherein the compound of Formula (I) is a compound of Formula (I-d): 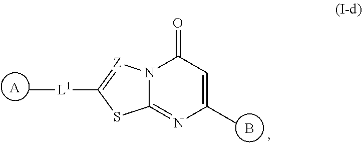
or a pharmaceutically acceptable salt, solvate, hydrate, tautomer, or stereoisomer thereof, wherein A, B, L¹, Z, and subvariables thereof are as defined in claim 1.

45. The compound of any one of the preceding claims, wherein the compound of Formula (I) is a compound of Formula (I-e): 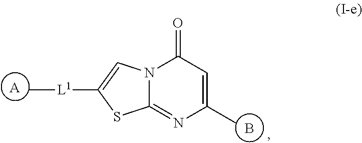
or a pharmaceutically acceptable salt, solvate, hydrate, tautomer, or stereoisomer thereof, wherein A, B, L¹, and subvariables thereof are as defined in claim 1.

46. The compound of any one of the preceding claims, wherein the compound of Formula (I) is a compound of Formula (I-f): 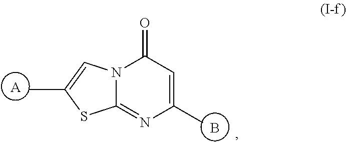
or a pharmaceutically acceptable salt, solvate, hydrate, tautomer, or stereoisomer thereof, wherein A is a nitrogen-containing 5-6 membered heterocyclyl or 5-6 membered cycloalkyl, each of which is optionally substituted with one or more R¹; and B is a bicyclic nitrogen-containing heteroaryl, optionally substituted with one or more R¹.

47. The compound of any one of the preceding claims, wherein the compound of Formula (I) is a compound of Formula (I-g): 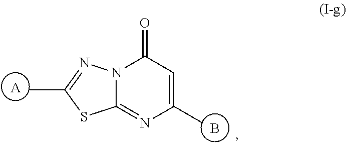
or a pharmaceutically acceptable salt, solvate, hydrate, tautomer, or stereoisomer thereof, wherein A is a nitrogen-containing 5-6 membered heterocyclyl or 5-6 membered cycloalkyl, each of which is optionally substituted with one or more R¹; and B is a bicyclic nitrogen-containing heteroaryl, optionally substituted with one or more R¹.

48. The compound of any one of the preceding claims, wherein the compound of Formula (I) is a compound of Formula (I-h): 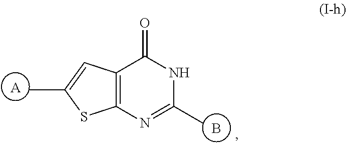
or a pharmaceutically acceptable salt, solvate, hydrate, tautomer, or stereoisomer thereof, wherein A is a nitrogen-containing 5-6 membered heterocyclyl or 5-6 membered cycloalkyl, each of which is optionally substituted with one or more R¹; and B is a bicyclic nitrogen-containing heteroaryl, optionally substituted with one or more R¹.

49. The compound of any one of the preceding claims, wherein the compound of Formula (I) is selected from any one of the compounds shown in Table 1 or a pharmaceutically acceptable salt, solvate, hydrate, tautomer, or stereoisomer thereof.

50. A compound of Formula (II): 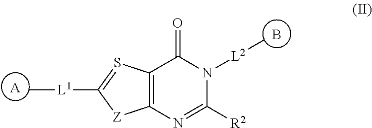
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
L¹ and L² are each independently absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴;
Z is N or C(R⁶);
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
R² is hydrogen, C₁-C₆-alkyl, C₁-C₆-heteroalkyl, C₁-C₆-haloalkyl, halo, cyano, cycloalkyl, heterocyclyl, or —OR^{A};
each R³ is independently hydrogen, C₁-C₆-alkyl, or C₁-C₆-haloalkyl;
each R⁴ is independently C₁-C₆-alkyl, C₁-C₆-heteroalkyl, C₁-C₆-haloalkyl, cycloalkyl, heterocyclyl, halo, cyano, oxo, —OR^{A}, —NR^{B}R^{C}, —C(O)R^{D}, or —C(O)OR^{D};
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷;
R⁶ is hydrogen, C₁-C₆-alkyl, C₁-C₆-heteroalkyl, C₁-C₆-haloalkyl, halo, cyano, cycloalkyl, heterocyclyl, or —OR^{A};
each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A} is independently hydrogen, C₁-C₆ alkyl, C₁-C₆ haloalkyl, C₁-C₆ heteroalkyl, aryl, heteroaryl, C₁-C₆ alkylene-aryl, C₁-C₆ alkylene-heteroaryl, cycloalkyl, heterocyclyl, —C(O)R^{D}, or —S(O)ₓR^{D};
each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, —OR^{A}, or —S(O)ₓR^{D}; or
R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁹ is independently C₁-C₆-alkyl or halo; and
x is 0, 1, or 2.

51. The compound of claim 50, wherein one of A and B is independently heterocyclyl, heteroaryl, or cycloalkyl.

52. The compound of any one of claims 50-51, wherein one of A and B is independently a nitrogen-containing heterocyclyl or nitrogen-containing heteroaryl.

53. The compound of any one of claims 50-52, wherein one of A and B is independently selected from 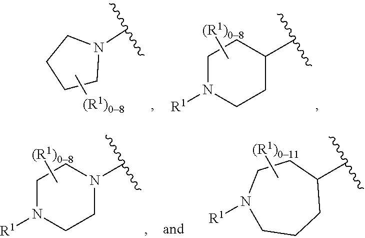
wherein R¹ is as described in claim 50.

54. The compound of any one of claims 50-54, wherein one of A and B is independently 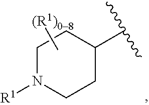
wherein R¹ is as described in claim 50.

55. The compound of any one of claims 50-54, wherein one of A and B is independently selected from

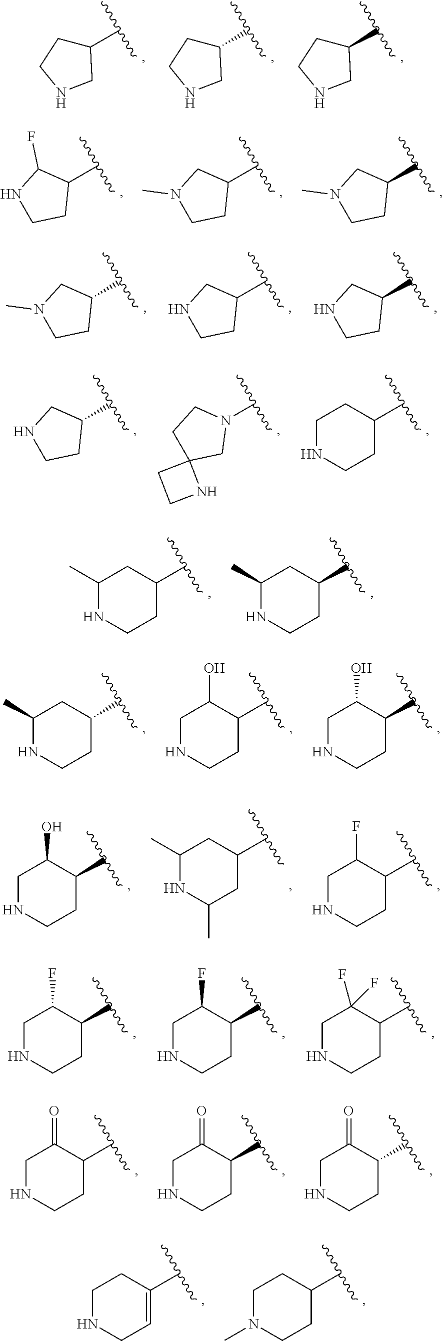

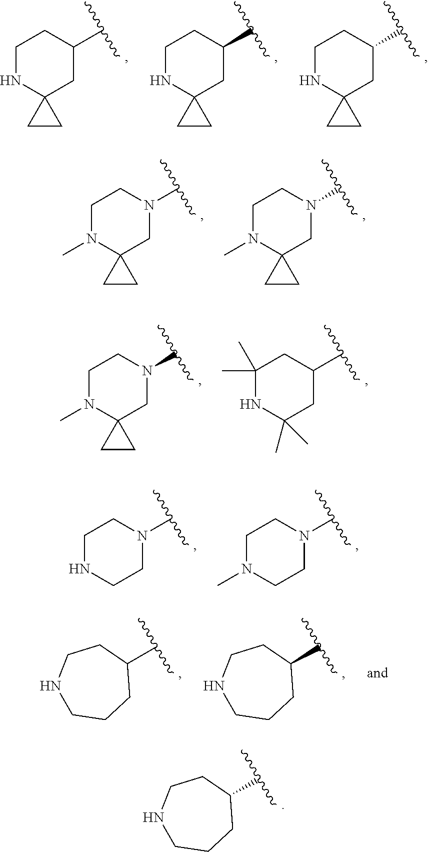

56. The compound of any one of claims 50-55, wherein one of A and B is independently selected from

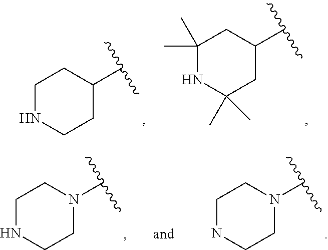 [SMILES: *C1CCNCC1 ; *C1CC(C)(C)NC(C)(C)C1 ; *N1CCNCC1 ; *N1CCNCC1]

57. The compound of any one of claims 50-56, wherein A is nitrogen-containing heterocyclyl.

58. The compound of any one of claims 38-43, wherein A is selected from 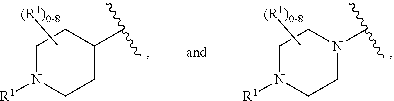
wherein R¹ is as described in claim 50.

59. The compound of any one of claims 50-58, wherein A is

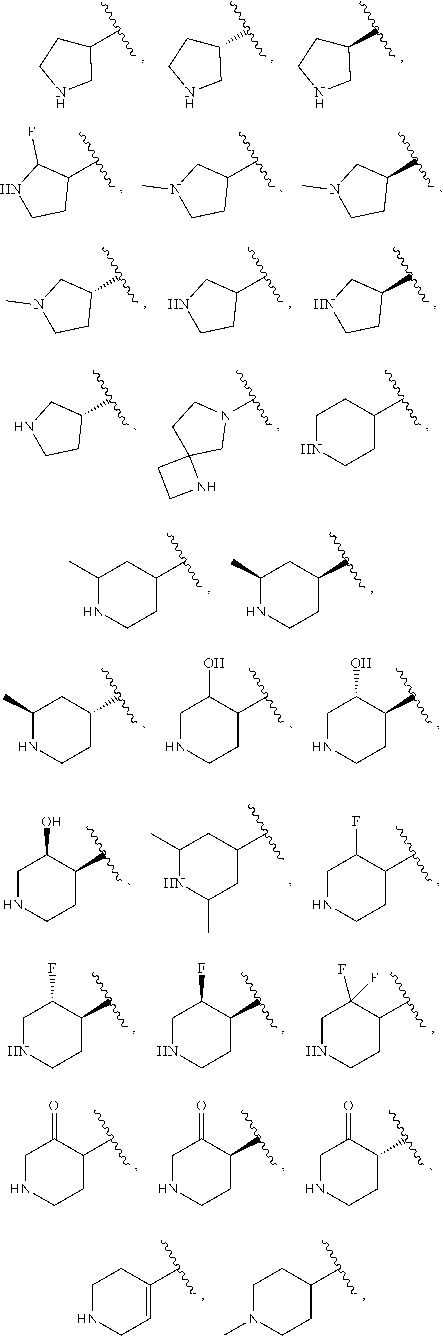

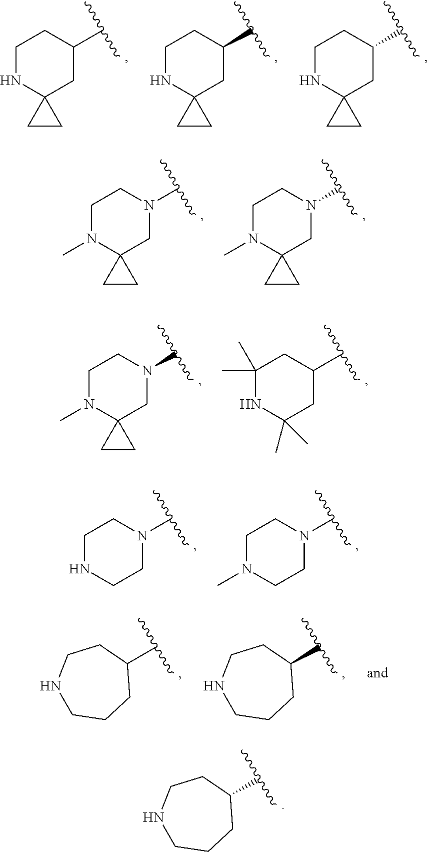

60. The compound of any one of claims 50-51, wherein one of A and B is independently cycloalkyl.

61. The compound of claim 60, wherein one of A and B is independently 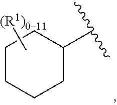
wherein R¹ is as described in claim 50.

62. The compound of any one of claims 60-61, wherein one of A and B is independently selected from 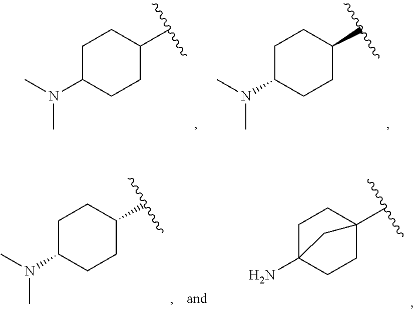
wherein R¹ is as described in claim 61.

63. The compound of any one of claims 50-62, wherein one of A and B is independently selected from 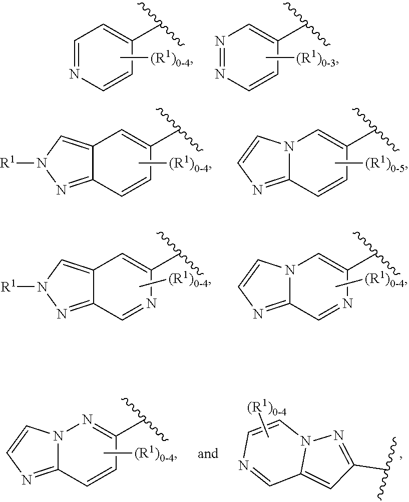
wherein R¹ is as described in claim 50.

64. The compound of any one of claims 50-63, wherein one of A and B is independently selected from 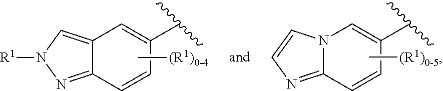
wherein R¹ is as described in claim 50.

65. The compound of any one of claims 50-64, wherein one of A and B is independently selected from

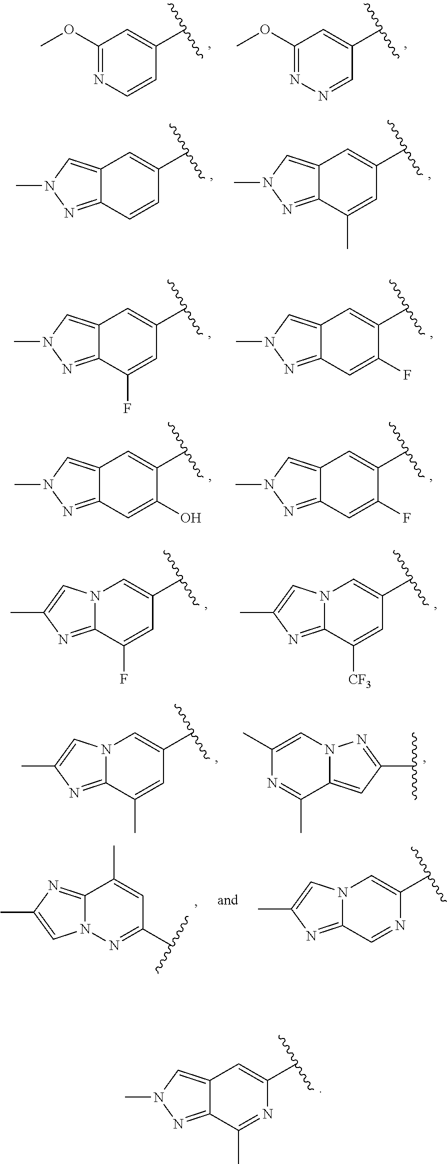

66. The compound of any one of claims 50-65, wherein one of A and B is independently selected from,

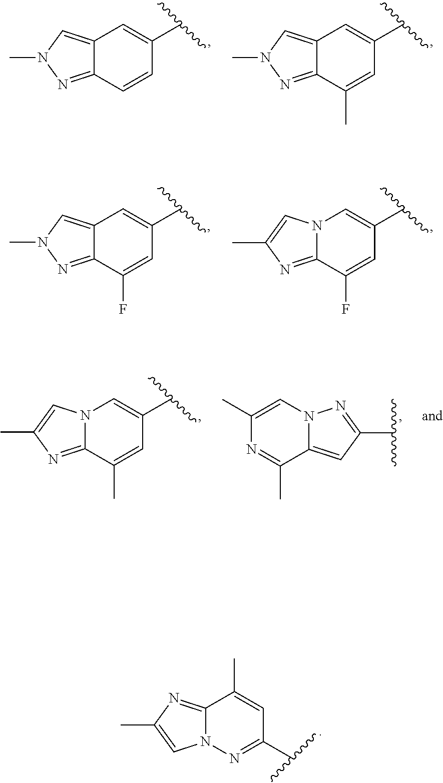

67. The compound of any one of claims 50-66, wherein one of A and B is independently selected from

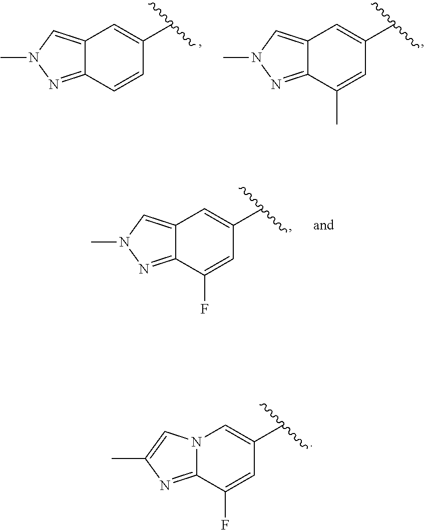 [SMILES: *c1ccc2nn(C)cc2c1 ; *c1cc(C)c2nn(C)cc2c1 ; *c1cc(F)c2nn(C)cc2c1 ; *c1cc(F)c2nc(C)cn2c1]

68. The compound of any one of claims 50-67, wherein B is a nitrogen-containing heteroaryl.

69. The compound of any one of claims 50-68, wherein B selected from selected from 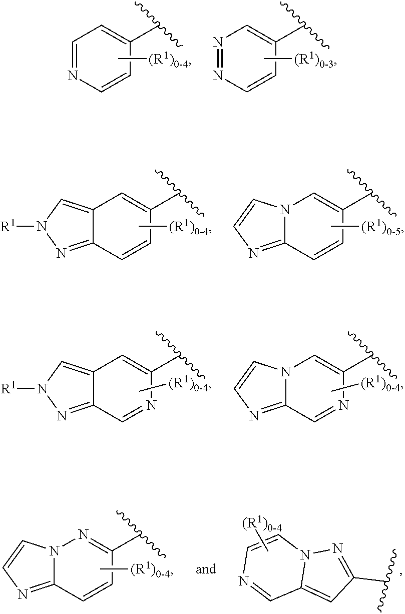
wherein R¹ is as described in claim 50.

70. The compound of any one of claims 50-69, wherein B is selected from

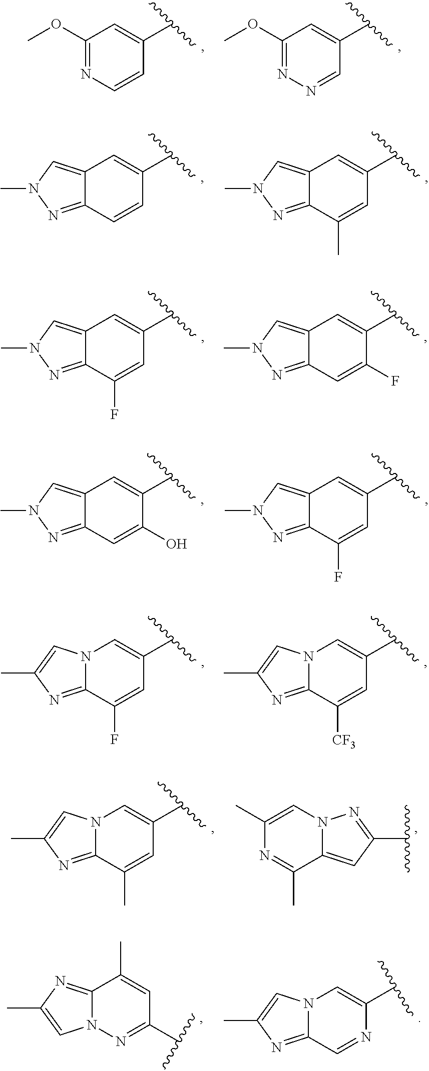

71. The compound of any one of claims 50-70, wherein one of L¹ and L² is independently absent or C₁-C₆-heteroalkylene (e.g., —N(CH₃)—).

72. The compound of any one of claims 50-71, wherein one of L¹ and L² is independently absent.

73. The compound of any one of claims 50-72, wherein one of L¹ and L² is independently absent.

74. The compound of any one of claims 50-73, wherein Z is N.

75. The compound of any one of claims 50-74, wherein Z is CH.

76. The compound of any one of claims 50-75, wherein R² is hydrogen.

77. The compound of any one of claims 50-76, wherein the compound of Formula (II) is a compound of Formula (II-a): 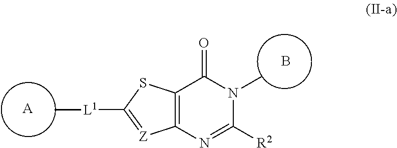
or a pharmaceutically acceptable salt, solvate, hydrate, tautomer, or stereoisomer thereof, wherein A, B, L¹, Z, R², and subvariables thereof are as defined in claim 50.

78. The compound of any one of claims 50-77, wherein the compound of Formula (II) is a compound of Formula (II-b): 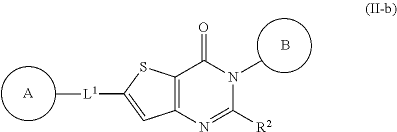
or a pharmaceutically acceptable salt, solvate, hydrate, tautomer, or stereoisomer thereof, wherein A, B, L¹, R², and subvariables thereof are as defined in claim 50.

79. The compound of any one of claims 50-78, wherein the compound of Formula (II) is a compound of Formula (II-c): 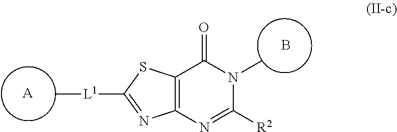
or a pharmaceutically acceptable salt, solvate, hydrate, tautomer, or stereoisomer thereof, wherein A, B, L¹, R², and subvariables thereof are as defined in claim 50.

80. The compound of any one of claims 50-79, wherein the compound of Formula (II) is selected from any one of the compounds shown in Table 2 or a pharmaceutically acceptable salt, solvate, hydrate, tautomer, or stereoisomer thereof.

81. A compound of Formula (III): 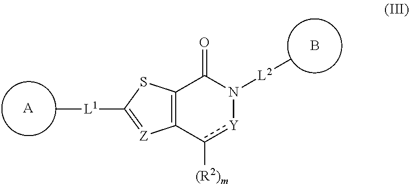
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
L¹ and L² are each independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵;
Y is N, N(R^{3a}), C(R^{3b}), or C(R^{3b})(R^{3c}), wherein the dashed lines representing bonds in the ring comprising Y may be single or double bonds as valency permits;
Z is N or C(R⁶);
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷;
each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D};
R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —C(O)R^{D}, or —S(O)ₓR^{D};
R^{3b} and R^{3c} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D};
R⁴ is hydrogen, C₁-C₆-alkyl, or C₁-C₆-haloalkyl;
R⁵ is C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
R⁶ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D};
each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R′;
each R⁸ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each R^{B} and R^{C} is independently hydrogen, C₁-C₆ alkyl, C₁-C₆ heteroalkyl, cycloalkyl, heterocyclyl, —OR^{A}; or
R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁹ is independently C₁-C₆-alkyl or halo;
m is 1 or 2, wherein when the dashed lines representing bonds in the ring comprising Y are single bonds, m is 2, and when the dashed lines representing bonds in the ring comprising Y are double bonds, m is 1; and
x is 0, 1, or 2.

82. The compound of claim 81, wherein one of A and B is independently heterocyclyl, heteroaryl, or cycloalkyl.

83. The compound of any one of claims 81-82, wherein one of A and B is independently a nitrogen-containing heterocyclyl or nitrogen-containing heteroaryl.

84. The compound of any one of claims 81-83, wherein one of A and B is independently selected from 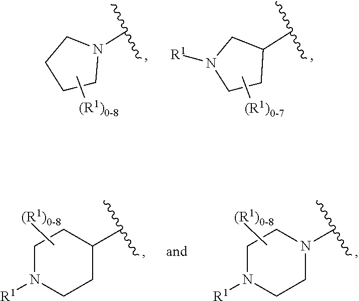
wherein R¹ is as described in claim 81.

85. The compound of any one of claims 81-84, wherein one of A and B is independently 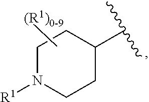
wherein R¹ is as described in claim 81.

86. The compound of any one of claims 81-85, wherein B is independently a nitrogen-containing heterocyclyl.

87. The compound of any one of claims 81-86, wherein B is independently 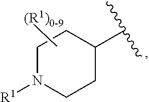
wherein R¹ is as described in claim 81.

88. The compound of any one of claims 81-87, wherein one of A and B is independently selected from

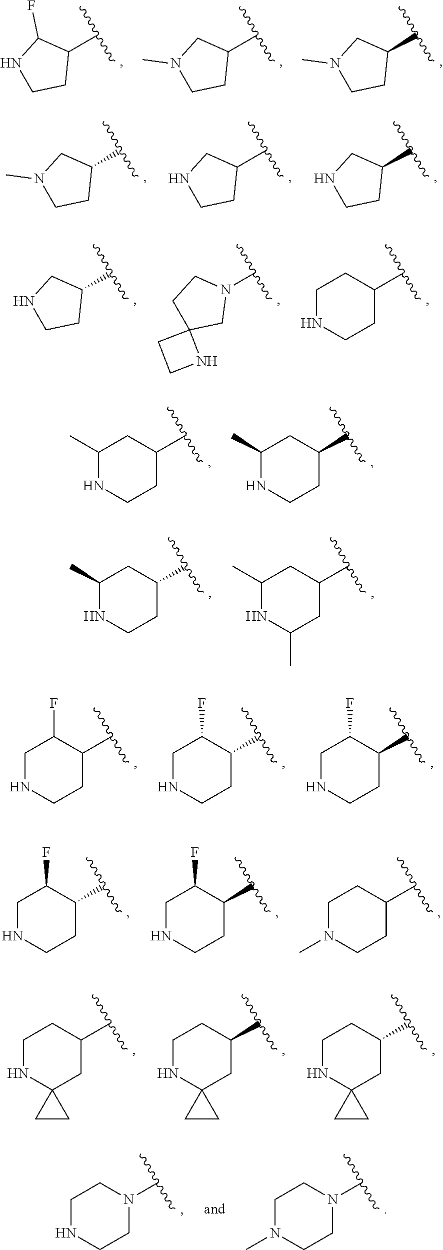

89. The compound of any one of claims 81-88, wherein one of A and B is independently selected from

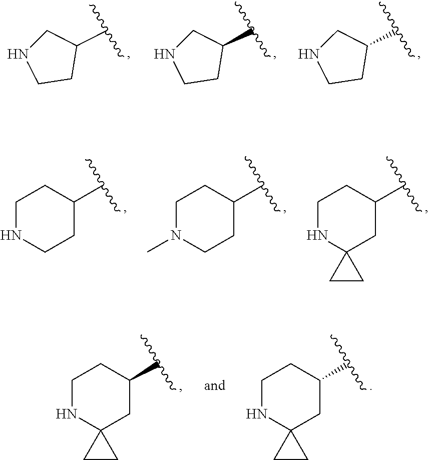

90. The compound of any one of claims 81-89, wherein one of A and B is independently selected from

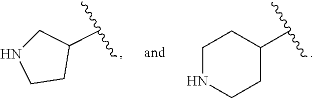 [SMILES: *C1CCNC1 ; *C1CCNCC1]

91. The compound of any one of claims 81-90, wherein one of A and B is independently

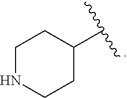 [SMILES: *C1CCNCC1]

92. The compound of any one of claims 66-76, wherein B is selected from

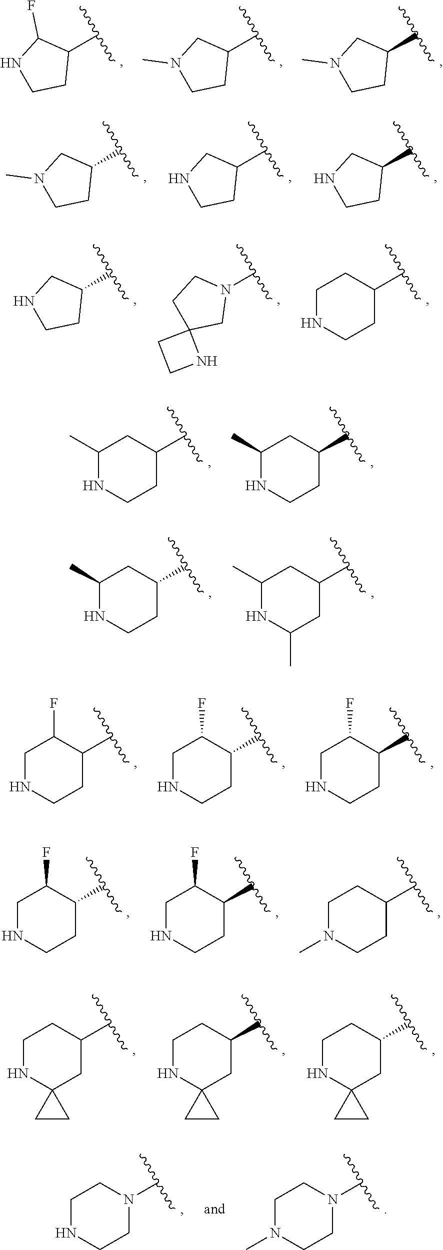

93. The compound of any one of claims 81-92, wherein B is

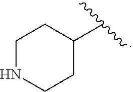 [SMILES: *C1CCNCC1]

94. The compound of any one of claims 81-82, wherein one of A and B is independently cycloalkyl.

95. The compound of claim 94, wherein one of A and B is independently 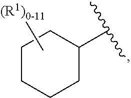
wherein R¹ is as described in claim 81.

96. The compound of any one of claims 94-95, wherein one of A and B is independently selected from 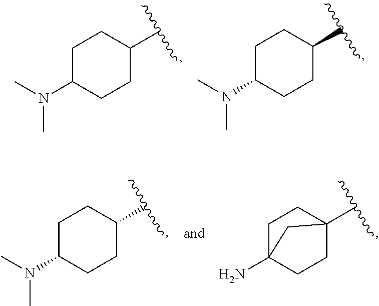
wherein R¹ is as described in claim 81.

97. The compound of any one of claims 81-96, wherein one of A and B is independently selected from 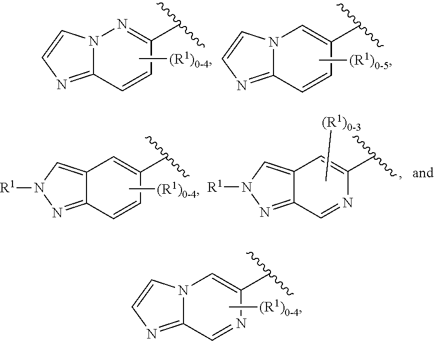
wherein R¹ is as described in claim 81.

98. The compound of any one of claims 81-97, wherein one of A and B is independently selected from 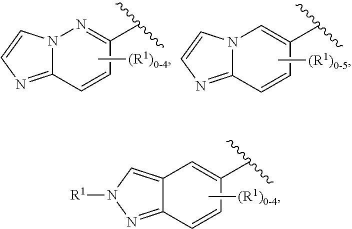
wherein R¹ is as described in claim 81.

99. The compound of any one of claims 81-98, wherein A is a nitrogen-containing heteroaryl.

100. The compound of any one of claims 81-99, wherein A is selected from 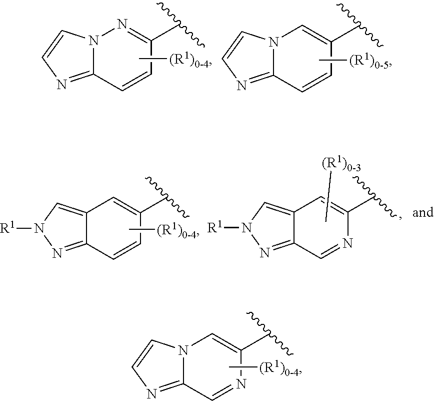
wherein R¹ is as described in claim 81.

101. The compound of any one of claims 81-100, wherein one of A and B is independently elected from

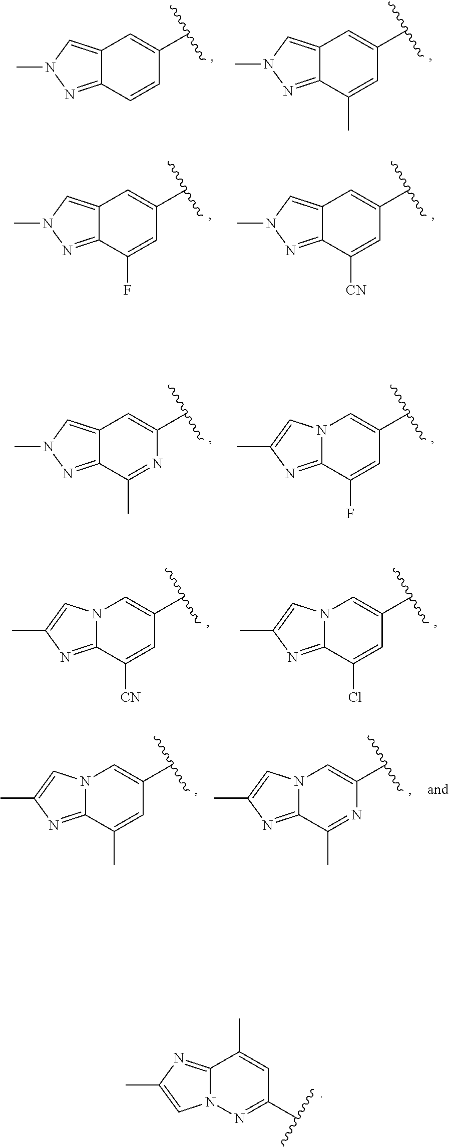

102. The compound of any one of claims 81-101, wherein one of A and B is independently selected from

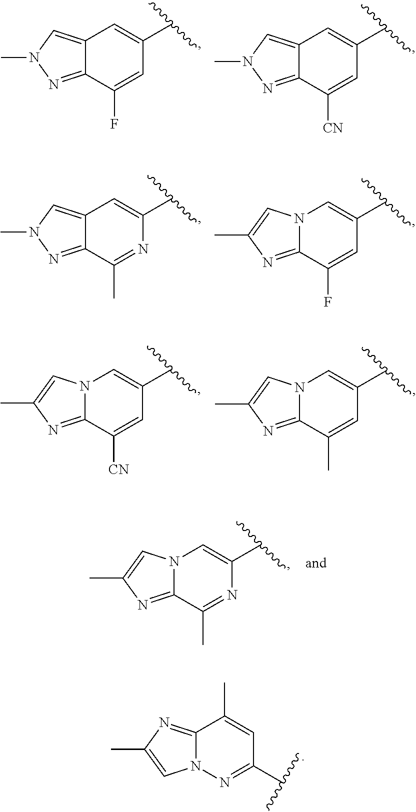

103. The compound of any one of claims 81-102, wherein A is selected from

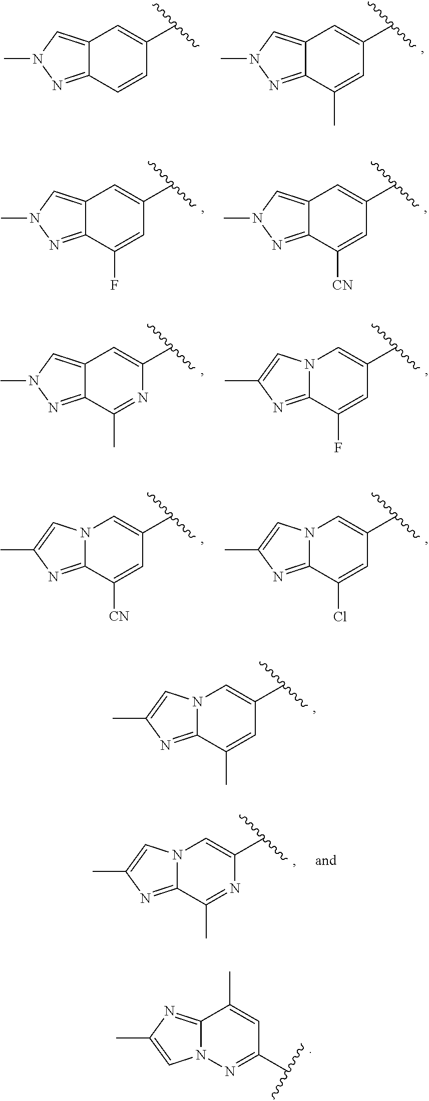

104. The compound of any one of claims 81-103, wherein A is selected from

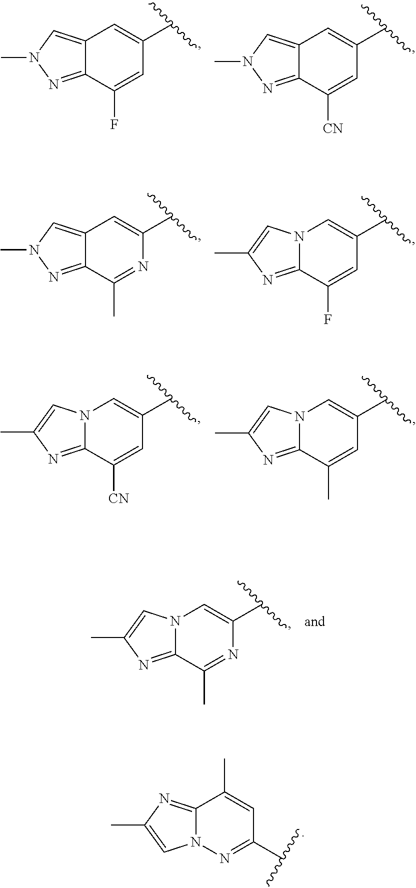

105. The compound of any one of claims 81-104, wherein one of L¹ and L² is independently absent.

106. The compound of any one of claims 81-104, wherein L¹ is absent.

107. The compound of any one of claims 81-105, wherein L² is absent.

108. The compound of any one of claims 81-107, wherein each of L¹ and L² is independently absent.

109. The compound of any one of claims 81-108, wherein Z is C(R⁶) (e.g., CH).

110. The compound of any one of claims 81-108, wherein Z is N.

111. The compound of any one of claims 81-110, wherein Y is C(R^{3b}) (e.g., CH).

112. The compound of claim 111, wherein m is 1.

113. The compound of claim 111, wherein m is 2.

114. The compound of any one of claims 81-113, wherein Y is N.

115. The compound of any one of claims 81-114, wherein R² is hydrogen or alkyl (e.g., CH₃).

116. The compound of any one of claims 81-115, wherein R² is hydrogen.

117. The compound of any one of claims 81-116, wherein the compound of Formula (III) is a compound of Formula (III-a): 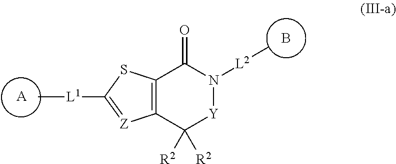
or a pharmaceutically acceptable salt, solvate, hydrate, tautomer, or stereoisomer thereof, wherein A, B, L¹, L², Y, Z, R², and subvariables thereof are as defined in claim 81.

118. The compound of any one of claims 81-117, wherein the compound of Formula (III) is a compound of Formula (III-b): 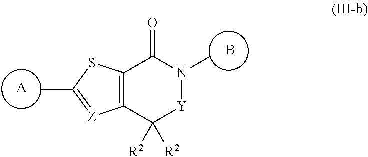
or a pharmaceutically acceptable salt, solvate, hydrate, tautomer, or stereoisomer thereof, wherein A, B, R², and subvariables thereof are as defined in claim 81.

119. The compound of claim 118, wherein A is a nitrogen-containing heteroaryl and B is a nitrogen-containing heterocyclyl, wherein each heterocyclyl and heteroaryl is optionally substituted with one or more R¹.

120. The compound of any one of claims 81-119, wherein the compound of Formula (III) is a compound of Formula (III-c): 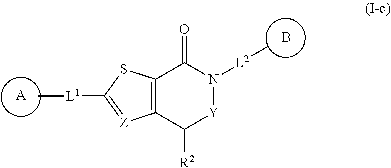
or a pharmaceutically acceptable salt, solvate, hydrate, tautomer, or stereoisomer thereof, wherein A, B, L¹, L², Y, Z, R², and subvariables thereof are as defined in claim 81.

121. The compound of claim 120, wherein A is a nitrogen-containing heteroaryl and B is a nitrogen-containing heterocyclyl, wherein each heterocyclyl and heteroaryl is optionally substituted with one or more R¹.

122. The compound of any one of claims 81-121, wherein the compound of Formula (III) is a compound of Formula (III-d): 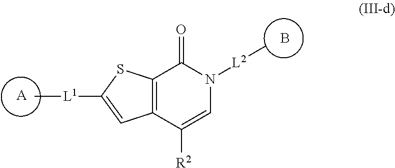
or a pharmaceutically acceptable salt, solvate, hydrate, tautomer, or stereoisomer thereof, wherein A, B, L¹, L², R², and subvariables thereof are as defined in claim 81.

123. The compound of claim 122, wherein A is a nitrogen-containing heteroaryl and B is a nitrogen-containing heterocyclyl, wherein each heterocyclyl and heteroaryl is optionally substituted with one or more R¹.

124. The compound of any one of claims 81-123, wherein the compound of Formula (III) is a compound of Formula (III-e): 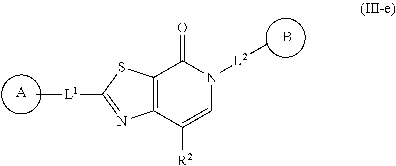
or a pharmaceutically acceptable salt, solvate, hydrate, tautomer, or stereoisomer thereof, wherein A, B, L¹, L², R², and subvariables thereof are as defined in claim 81.

125. The compound of claim 124, wherein A is a nitrogen-containing heteroaryl and B is a nitrogen-containing heterocyclyl, wherein each heterocyclyl and heteroaryl is optionally substituted with one or more R¹.

126. The compound of any one of claims 66-107, wherein the compound of Formula (III) is a compound of Formula (III-f): 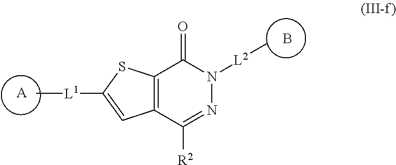
or a pharmaceutically acceptable salt, solvate, hydrate, tautomer, or stereoisomer thereof, wherein A, B, L¹, L², R², and subvariables thereof are as defined in claim 81.

127. The compound of claim 126, wherein A is a nitrogen-containing heteroaryl and B is a nitrogen-containing heterocyclyl, wherein each heterocyclyl and heteroaryl is optionally substituted with one or more R¹.

128. The compound of any one of claims 81-127, wherein the compound of Formula (III) is a compound of Formula (III-g): 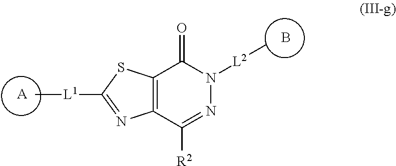
or a pharmaceutically acceptable salt, solvate, hydrate, tautomer, or stereoisomer thereof, wherein A, B, L¹, L², R², and subvariables thereof are as defined in claim 6816.

129. The compound of claim 110, wherein A is a nitrogen-containing heteroaryl and B is a nitrogen-containing heterocyclyl, wherein each heterocyclyl and heteroaryl is optionally substituted with one or more R¹.

130. The compound of any one of claims 81-129, wherein the compound is selected from any one of the compounds shown in Table 3 or a pharmaceutically acceptable salt, solvate, hydrate, tautomer, or stereoisomer thereof.

131. A pharmaceutical composition comprising a compound of any one of claims 1-130 and a pharmaceutically acceptable excipient.

132. The compound of any one of claims 1-130 or the pharmaceutical composition of claim 131, wherein the compound alters a target nucleic acid (e.g., an RNA, e.g., a pre-mRNA).

133. The compound of any one of claims 1-130 or the pharmaceutical composition of claim 131, wherein the compound binds to a target nucleic acid (e.g., an RNA, e.g., a pre-mRNA).

134. The compound of any one of claims 1-130 or the pharmaceutical composition of claim 131, wherein the compound stabilizes a target nucleic acid (e.g., an RNA, e.g., a pre-mRNA).

135. The compound of any one of claims 1-130 or the pharmaceutical composition of claim 131, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

136. The compound of any one of claims 1-112 or the pharmaceutical composition of claim 113, wherein the compound decreases splicing at splice site on a target nucleic acid (e.g., an RNA, e.g., a pre-mRNA), by about 0.5%, 1%, 2%, 3%, 4%, 5%, 6%, 7% 8%, 9% 10%, 15%, 20%, 25%, 30%, 35%, 40%, 45%, 50%, 55%, 60%, 65%, 70%, 75%, 80%, 85%, 90%, 95%, or more, e.g., as determined by qPCR %.

137. A method of modulating splicing of a nucleic acid (e.g., DNA, RNA, e.g., a pre-mRNA) comprising contacting the nucleic acid with a compound of Formula (I), (II), or (III) according to any one of claims 1-130 or the pharmaceutical composition of claim 131.

138. The method of claim 137, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

139. The method of claim 138, wherein the compound decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

140. A method of forming a complex comprising a component of a spliceosome (e.g., a major spliceosome component or a minor spliceosome component), a nucleic acid (e.g., a DNA, RNA, e.g., a pre-mRNA), and a compound of Formula (I), (II), or (III):
comprising contacting the nucleic acid (e.g., a DNA, RNA, e.g., a pre-mRNA) with a compound of Formula (I), (II), (III), according to any one of claims 1-130 or the pharmaceutical composition of claim 131.

140. The method of claim 139, wherein the component of a spliceosome is recruited to the nucleic acid in the presence of the compound of Formula (I), (II), or (III).

141. A method of altering the conformation of a nucleic acid (e.g., a DNA, RNA, e.g., a pre-mRNA) comprising contacting the nucleic acid with a compound of Formula (I), (II), or (III), according to any one of claims 1-130 or the pharmaceutical composition of claim 131.

142. The method of claim 141, wherein the altering comprises forming a bulge in the nucleic acid.

143. The method of claim 141, wherein the altering comprises stabilizing a bulge in the nucleic acid.

144. The method of claim 141, wherein the altering comprises reducing a bulge in the nucleic acid.

145. The method of any one of claims 124-127, wherein the nucleic acid comprises a splice site.

146. A method for treating a disease or disorder in a subject comprising administering to the subject a compound of Formula (I), (II), or (III), according to any one of claims 1-130 or the pharmaceutical composition of claim 131.

147. The method of claim 146, wherein the disease or disorder comprises a proliferative disease (e.g., cancer, a benign neoplasm, or angiogenesis).

148. The method of claim 147, wherein the proliferative disease is cancer.

149. The method of claim 148, wherein the cancer is selected form adenoid cystic carcinoma, colorectal cancer, leukemia, lung cancer, prostate cancer, or ovarian cancer.

150. The method of claim 149, wherein the disease or disorder comprises a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

151. The method of claim 150, wherein the disease or disorder comprises neurological disease or disorder.

152. The method of claim 151, wherein the disease or disorder comprises Huntington's disease.

153. A composition for use in treating a disease or disorder in a subject comprising administering to the subject a compound of Formula (I), (II), or (III), according to any one of claims 1-130 or the pharmaceutical composition of claim 131.
